SEQUENCE LISTING (82 sequences)

SEQ ID NO: 1 - DNA, 2949 bp, Homo sapiens
  source 1..2949: mol_type = genomic DNA
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 2 - protein, 765 aa, Homo sapiens
AALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 3 - DNA, 3906 bp, Rattus norvegicus
  source 1..3906: mol_type = genomic DNA
atggcggcgctgagtggcggcggtggcagcagcagcggtggcggtggcggcggcggcggcggcggtggtggcggcggcggcggcggcgccgaacagggacaggctctgttcaatggcgacatggagccggaggccggcgctggcgccgcggcctcttcggccgcggacccggccattcctgaagaggtgtggaatatcaagcaaatgattaagttgacacaggaacatatagaggccctattggacaagtttggtggggagcataacccaccgtcaatatacctggaggcctatgaagagtacaccagcaagctagatgcccttcagcagagagagcagcagctgttggaatccctggtttttcaaactcccacagatgtatcacggaacaaccccaagtcaccacagaaacctatcgttcgtgtcttcctgcccaacaaacagaggacagtggtgcccgcaagatgtggtgtaacggtccgagacagtctaaagaaagcactaatgatgaggggtctcatcccagagtgctgtgctgtttacagaattcaggacggagagaagaaaccaattggctgggacactgacatttcctggcttactggagaggagctacatgttgaagtactagagaatgttcctctgacaacccacaacttcgtacggaaaacttttttcaccttagcattttgtgacttttgccgaaagctgcttttccagggtttccgctgtcaaacatgtggttataagtttcaccagcgttgtagtacagaggttccactgatgtgtgttaattatgaccaacttgatttgctgtttgtctccaagttctttgagcatcacccagtaccacaggaggaggccttctcagcagagactacccttccatctggatgctcttccgcacccccctcagactctattgggccccaaatcctcaccagtccatctccttcaaaatccattccaattccacagcccttccggccagcagatgaagatcatcgcaatcagtttgggcaacgagaccgctcctcctccgctcccaatgttcatataaacacaatcgaacctgtcaatattgatgaaaaattcccagaagtggaattacaggatcaaagggatttgattagagaccaggggtttcgtggggatggagcccctttgaaccagctgatgcgctgtcttcggaaataccaatcccggactcccagccccctcctccattctgtccccagtgaaatagtgtttgattttgagcctggcccagtgttcagagggtcaaccacaggcttgtcggccaccccacctgcctcattacctggctcactcactaacgtgaaagccttacagaaatctccaggacctcagcgggaaaggaagtcctcctcctcctcctcctccacggaagacagaagtcggatgaaaacacttggtagaagagattcaagtgatgattgggagattcctgatggacagattacagtgggacagagaattggatctgggtcctttggaactgtctacaagggaaagtggcatggcgacgtggcagtgaaaatgctgaatgtgacagcacccacacctcagcagttacaggccttcaaaaacgaagtcggagtactcaggaaaactcgacatgtgaacatcctccttttcatgggctattctacaaagccacagctggctattgttacacagtggtgtgaaggctccagcttatatcaccatctccacatcattgagaccaaatttgagatgatcaaacttatagatattgcacggcagactgcacagggcatggattacttacacgccaagtcaatcatccacagagacctcaagagtaataatatatttcttcatgaagacctcacggtaaaaataggtgactttggtttagccacagtgaagtcccgatggagtgggtcccatcagtttgaacagttgtctggatctattttgtggatggcacccgaagtaatcagaatgcaagataaaaacccatatagctttcagtcagacgtgtatgcatttgggattgttctgtatgaactgatgactggtcagctaccttattcaaacatcaacaacagggatcagataatttttatggtgggacgaggatacctatctccagatctcagtaaggtacggagtaactgtccaaaagccatgaagagattaatggcagagtgcctcaaaaagaaaagagacgagagaccactctttccccaaattctcgcctctattgagctgctggcccgctcattgccaaaaattcaccgcagtgcatcagaaccctccttgaatcgggctggtttccaaacagaagattttagtctgtatgcttgtgcttctccaaaaacacccatccaagcagggggatatggagaatttgcagccttcaagtagccactccatcatggcagcatctactctttatttcttaagtcttgtgttcatacaatttgttaacatcaaaacacagttctgttcctcaaattttttttaaagatacaaaattttcaatgcataagctcgtgtggaacagaatggaatttcctattcaacaaaagagggaagaatgttttaggaaccagaattctctgctgcccgtgtttcttcttcaacacaaatatcatgtgcatacaactctgcccattcccaagaagaaagaggagagaccccgaattctgcccttttggtggtcaggcatgatggaaagaatttgctgctgcagcttgggaaaaattgctatggaaagtctgccagtcaactttgcccttctaaccaccagatccatttgtggctggtcatctgatggggcgatttcaatcaccaagcatcgttcttgcctgttgtgggattatgtcgtggagcactttccctatccaccaccgttaatttccgagggatggagtaaatgcagcataccctttgtgtagcacctgtccagtcctcaaccaatgctatcacagtgaagctctttaaatttaagtggtgggtgagtgttgaggagagactgccttgggggcagagaaaaggggatgctgcatcttcttcctcacctccagctctctcacctcgggttgccttgcacactgggctccgcctaaccactcgggctgggcagtgctggcacacattgccgccttttctcattgggtccagcaattgagcagagggttgggggattgtttcctccacaatgtagcaaattctcaggaaaatacagtccatatcttcctctcagctcttccagtcaccaaatacttacgtggctcctttgtccaggacataaaacaccgtggacaacacctaattaaaagcctacaaaactgcttactgacagttttgaatgtgagacatttgtgtaatttaaatgtaaggtacaggtcttaatttcttctattaagtttcttctatttttatttaaacgaagaaaataattttcaggtttaattggaataaacgaatacttcccaaaagactatataccctgaaaattatatttttgttaattgtaaacaacttttaaaaaatggttattatccttttctctacctaaaattatgggaaatcttagcataatgacaattatttatactttttaaataaatggtacttgctggatccacactaacatctttgctaacattcccattgtttcttccaacttcactcctacactacatcctccatcctctttctagtcttttatctataatatgcaacctaaaataaaagtggtggtgtctccattcattcttcttcttccttttttccccaagcctggtcttcaaaaggttgggtaatttagtagctgagttccctaggtagaaatagaactattagggacattggggttgtaggaaagcgtgaggcctgtcaccagttgttctt

SEQ ID NO: 4 - protein, 804 aa, Rattus norvegicus
MAALSGGGGSSSGGGGGGGGGGGGGGGGGAEQGQALFNGDMEPEAGAGAAASSAADPAIPEEVWNIKQMIKLTQEHIEALLDKFGGEHNPPSIYLEAYEEYTSKLDALQQREQQLLESLVFQTPTDVSRNNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPVPQEEAFSAETTLPSGCSSAPPSDSIGPQILTSPSPSKSIPIPQPFRPADEDHRNQFGQRDRSSSAPNVHINTIEPVNIDEKFPEVELQDQRDLIRDQGFRGDGAPLNQLMRCLRKYQSRTPSPLLHSVPSEIVFDFEPGPVFRGSTTGLSATPPASLPGSLTNVKALQKSPGPQRERKSSSSSSSTEDRSRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 5 - DNA, 9728 bp, Mus musculus
  source 1..9728: mol_type = genomic DNA
ccctcaggctcggctgcgccggggccgccggcgggttccagaggtggcctccgccccggccgctccgcccacgccccccgcgcctccgcgcccgcctccgcccgccctgcgcctcccttccccctccccgccccgcggcggccgctcggcccggctcgcgcttcgaagatggcggcgctgagtggcggcggtggcagcagcagcggtggcggcggcggcggtggcggcggcggtggcggtggcgacggcggcggcggcgccgagcagggccaggctctgttcaatggcgacatggagccggaggccggcgctggcgccgcggcctcttcggctgcggacccggccattcctgaagaggtatggaatatcaagcaaatgattaagttgacacaggaacatatagaggccctattggacaaatttggtggagagcataacccaccatcaatatacctggaggcctatgaagagtacaccagcaagctagatgcccttcagcaaagagaacagcagcttttggaatccctggtttttcaaactcccacagatgcatcacggaacaaccccaagtcaccacagaaacctatcgttagagtcttcctgcccaacaaacagaggacagtggtacccgcaagatgtggtgttacagttcgagacagtctaaagaaagcactgatgatgagaggtctcatcccagaatgctgtgctgtttacagaattcaggatggagagaagaaaccaattggctgggacacggacatttcctggcttactggagaggagttacatgttgaagtactggagaatgtcccacttacaacacacaactttgtacggaaaacttttttcaccttagcattttgtgacttttgccgaaagctgcttttccagggtttccgttgtcaaacatgtggttataaatttcaccagcgttgtagtacagaggttccactgatgtgtgtaaattatgaccaacttgatttgctgtttgtctccaagttctttgagcatcacccagtaccacaggaggaggcctccttcccagagactgcccttccatctggatcctcttccgcacccccctcagactctactgggccccaaatcctcaccagtccatctccttcaaaatccattccaattccacagcccttccgaccagcagatgaagatcatcgcaatcagtttgggcaacgagaccggtcctcctcagctcccaatgttcatataaacacaattgagcctgtgaatatcgatgaaaaattcccagaagtggaattacaggatcaaagggatttgattagagaccaggggtttcgtggtgatggagcccccttgaaccaactgatgcgctgtcttcggaaataccaatcccggactcccagccccctcctccattctgtccccagtgaaatagtgtttgattttgagcctggcccagtgttcagagggtcaaccacaggcttgtccgccaccccgcctgcctcattacctggctcactcactaacgtgaaagccttacagaaatctccaggtcctcagcgggaaaggaagtcatcttcttcctcatcctcggaggacagaagtcggatgaaaacacttggtagaagagattcaagtgatgactgggagattcctgatggacagattacagtgggacagagaattggatctgggtcatttggaactgtctacaagggaaagtggcatggtgatgtggcagtgaaaatgttgaatgtgacagcacccacacctcaacagctacaggccttcaaaaatgaagtaggagtgctcaggaaaactcgacatgtgaatatcctccttttcatgggctattctacaaagccacaactggcaattgttacacagtggtgtgagggctccagcttatatcaccatctccacatcattgagaccaaatttgagatgatcaaacttatagatattgctcggcagactgcacagggcatggattacttacacgccaagtcaatcatccacagagacctcaagagtaataatatatttcttcatgaagacctcacggtaaaaataggtgactttggtctagccacagtgaaatctcggtggagtgggtcccatcagtttgaacagttgtctggatctattttgtggatggcaccagaagtaatcagaatgcaagataaaaacccgtatagctttcagtcagacgtgtatgcgtttgggattgttctgtacgaactgatgaccggccagctaccttattcaaacatcaacaacagggatcagataatttttatggtgggacgaggatacctatctccagatctcagtaaggtacggagtaactgtccaaaagccatgaagagattaatggcagagtgcctcaaaaagaaaagagacgagagaccactctttccccaaattctcgcctccattgagctgctggcccgctcattgccaaaaattcaccgcagtgcatcagaaccttccttgaatcgggctggtttccaaacagaagattttagtctgtatgcttgtgcttctccgaaaacacccatccaagcagggggatatggagaatttgcagccttcaagtagccagtccatcatggcagcatctactctttatttcttaagtcttgtgttcatacagtttgttaacatcaaaacacagttctgttcctcaaaaaattttttaaagatacaaaattttcaatgcataagttcatgtggaacagaatggaatttcctattcaacaaaagagggaagaatgttttaggaaccagaattctctgctgcccgtgtttcttcttcaacataactatcacgtgcatacaagtctgcccattcccaagaagaaagaggagagaccctgaattctgcccttttggtggtcaggcatgatggaaagaatttgctgctgcagcttgggaaaattgctatggaaagtctgccagtcgactttgcccttctaaccaccagatcagcctgtggctggtcatctgatggggcgatttccatcaccaagcatcgttcttgcctattctgggattatgttgtggagcactttccctgtccagcaccgttcatttctgagggatggagtaaatgcagcattcccttgtgtagcgcctgttcagtcctcagcagctgctgtcacagcgaagctttttacagttaagtggtgggggagagttgaggagagcctgcctcggggcagagaaaagggggtgctgcatcttcttcctcacctccagctctctcacctcgggttgccttgctcactgggctccgcctaaccactcaggctgctcagtgctggcacacattgccttcttttctcattgggtccagcaattgaggagagggttgggggattgtttcctcctcaatgtagcaaattctcaggaaaatacagtccatatcttcctctcagctcttccagtcaccaaatacttacgtggctcctttgtccaggacataaaacaccgtggacaacacctaattaaaagcctacaaaactgcttactgacagttttgaatgtgagacacttgtgtaatttaaatgtaaggtacaggttttaatttctgagtttcttctatttttatttaaaagaagaaaataattttcagttttaattggaataaatgagtacttcccacaagactatataccctgaaaattatatttttgttaattgtaaacaacttttaaagaataattattatccttttctctacctaaaaattatggggaatcttagcataatgacaattatttatactttttaaataaatggtacttgctggatccacactaacatctttgctaacaatcccattgtttcttccaacttaactcctacactacatcctacatcctctttctagtcttttatctataatatgcaacctaaaataaacgtggtggcgtctccattcattctccctcttcctgttttccccaagcctggtcttcaaaaggttgggtaatcggtccctgagctccctagctggcaatgcaactattagggacattggagttgcaggagagcaggaagcctgtccccagctgttcttctagaaccctaaatcttatctttgcacagatcaaaagtatcacctcgtcacagttctccttagcctttacttacaggtaatataaataaaaatcaccatagtagtaaagaaaacaactggatggattgatgaccagtacctctcagagccaggaatcttgaatctccaggatttatacgtgcaaatttaaggagatgtacttagcaacttcaagccaagaacttccaaaatactagcgaatctaaaataaaatggaattttgagttatttttaaagttcaaattataattgataccactatgtatttaagcctactcacagcaagttagatggattttgctaaactcattgccagactgtggtggtggtggtggtagtgtgcacctttaatccaagcaactcagcaatcagaatgaggtaaatctctgtgaatacaaggcctgcctagtctgcagcgctagttccaggatagccagggctacacacacaaaaaccctctctcaaaaaaaacaaaattaattagttgataataaaaaataactaaagtatcatcaaaggaaggcctactggaagttttatatattcccagtaaattgaaaaatattctgaagttattaaccagttagcaacaatgtgtttttaagtcttacataaacagagcaaagtcttcaaatgtttcagagctgagaagataattgtgcttgatatgaaaaatagcctctccatatgatgtgccacattgaaaggcgtcattacccttttaaatacttcttaatgtggctttgttccctttacccaggattagctagaaagagctaggtaggcttcggccacagttgcacatttcgggcctgctgaagaatgggagctttgaaggctggccttggtggaggagcccctcagtgctggagggtggggcgtgtacgcagcatggaagtggtctagacagagtgcaaagggacagacttctttctcattttagtatagggtgatgtctcacttgaaatgagaaagtagagttgatattaaacgaagctgtgcccagaaaccaggctcagggtattgtgagattttctttttaaatagagaatataaaagatagaaataaatatttaaaccttccttcttattttctatcaaatagattttttttatcatttgcaaacaacataaaaaaaggtttcttttgtggggttttctttccttcttttttttttttttttttttttaagactgcagataatcttgttgagctcctcggaaaatacaaggaagtccgtgtttgtgcagagcgctttatgagtaactgtatagacagtgtggctgcttcactcatcccagagggctgcagctgtcggcccatgaagtggctgcagtgcctcgtgagatctgctttgttttgtttggagtgaagtctttgaaaggtttgagtgcaactatataggactgtttttaaataagtagtattcctcatgaactttctcattgttaagctacaggacccaaactctaccactaagatattattaacctcaaaatgtagtttatagaaggaatttgcaaatagaatatccagttcgtacttatatgcatcttcaacaaagattctctgtgacttgttggatttggttcctgaacagcccatttctgtatttgaggttaggagggcataatgaggcatcctaaaagacaatctgatataaactgtatgctagatgtatgctggtaggggagaaagcattctgtaaagacatgatttaagacttcagctctgtcaaccagaaaccttgtaaatacttcctgtcttggtgcagccccgcccctttgatcacacgatgttgtcttgtgcttgtcagacactgtcagagctgctgttcgtccctctgcagatctcacctgtccccactgcacacccacctcctgcctcttgcagacctcagcatctagctttagttggaaacagttcagggttcaggtgacttcttaaaaaaaaaaaaaaaccctacctcctcagaatgaggtaatgaatagttatttatttaaagtatgaagagtcaggagcgctcgaacatgaaggtgatttaagatggttcctttcgtgtgtattgtagctgagcacttgtttttgtcctaaagggcattatacatttaagcagtgattctgtttaaagatgtttttctttaaaggtgtagctcagagtatctgttgttggaattggtgccagagtctgcttaatagatttcagaatcctaagcttaagtcagtcgcatgaagttaagtagttatggtaacactttgctagccatgatataattctactttttaggagtaggtttggcaaaactgtatgccttcaaagtgagttggccacagctttgtcacatgcacagatactcatctgaagagactgcccagctaagagggcggaaggatacccttttttcctacgattcgcttctttgtccacgttggcattgttagtactagtttatcagcaccttgaccagcagatgtcaaccaataagctatttttaaaaccatagccagagatggagaggtcactgtgagtagaaacagcaggacgcttacaggagtgaaatggtgtagggaggctctagaaaaatatcttgacaatttgccaaatgatcttactgtgccttcatgatgcaataaaaaagctaacattttagcagaaatcagtgatttacgaagagagtggccagtctggtttaactcagctgggataatatttttagagtgcaatttagactgcgaagataaatgcactaaagagtttatagccaattcacatttgaaaaataagaaaatggtaaattttcagtgaaatatttttttaaagcacataatccctagtgtagccagaaatatttaccacatagagcagctaggctgagatacagtccagtgacatttctagagaaaccttttctactcccacgggctcctcaaagcatggaaattttatacaaaatgtttgacattttaagatactgctgtagtttagttttgaaatagtatgtgctgagcagcaatcatgtactaactcagagagagaaaacaacaacaaattgtgcatctgatttgttttcagagaaatgctgccaacttagatactgagttctcagagcttcaagtgtaaacttgcctcccaagtcctgtttgcaaatgaagttggctagtgctactgactgctccagcacatgatggaaggcagggggctgtctctgaagtgtcttctataaagggacaatagaatagtgagagacctggtcagtgtgtgtcagctggacactccatgctatgggacttgcatcttctgtcctcaccatccccaagacattgtgctttcctcagttgtcctctagctgtttcactcagacaccaagatgaattactgatgccagaaggggccaaaatggccagtgtgttttgggggttgtatcagttgactggacaataactttaatagtttcagatcatttatttttacttccattttgacagacatttaaatggaaatttagtcctaacttttgtcatttgaaaggaaaaattaacagttcctataagatacttttgaggtggaatctgacatcctaattttttttcttttcagtgggtttgcagcgagggtcttgtatgcactaggcaagggttctaccactaagccacatttcccaggaaataaaatgttaacagttaaaacatacacacaaatacacaaacaccttattaccactttagtaaagtgagagatgtgcgtcctttgtctcagtctccacgatttcagctgccccttgtatgaataactcagtctcgctaaactgtttacttttatttacctggtttgactagttgcagctatataaccagttgtgcatgaggacaacagccagtgtgtttgttttgtttttggttttttgtggtacattttttgtaaagaattctgtagattgaagtgctctttgaaaacagaactgagatatatttattcttgttagcatcaaaaaacattttgtgcaaatgatttgcttttcctggcaggctgagtaccatatccagcgcccacaattgcgggttcccatctaccatgtccacaggggagacagacgggaagcacatgaggggtgtgtttacagagttgtaggagttatgtagttctcttgttgccttggaaatcactgttgttttaagactgttgaacccgtgtgtttggctgggctgtgagttacatgaagaaactgcaaactagcatatgcagacaaagctcacagactaggcgtaaatggaggaaaatggaccaaaataaggcagggtgacacataaaccttgggcttcggagaaaactaagggtggagatgaactataatcacctgaatacaatgtaagagtgcaataagtgtgcttattctaagctgtgaacttcttttaaatcattcctttctaatacatttatgtatgttccattgctgactaaaaccagctatgagaacatatgcctttttattcatgttaactaccagtttaagtggctaaccttaatgtcttatttatcttcattttgtattagtttacataccaggtatgtgtgtgtgctgtactcttcttccctttatttgaaaacacttttcactgggtcatctccttggccattccacaacacaactttggtttggctttcaatgtcaccttatttgatggcctgtgtcccagtagcagaatttatggtattcccattgctggctgctcttccgaccctttgcttctacagcacttgtctctcctaagatagtcagaaactaactgatcaggggatggacttcaccattcatcgtgtctcttcaattctattaaatagaccactcttgggctttagaccaggaaaaaggagacagctctagccatctaccaagcctcaccctaaaaggtcacccgtacttcttggtctgaggacaagtctccactccagtaagggagaggggaggaaatgcttcctgtttgaaatgcagtgaattcctatggctcctgtttcaccacccgcacctatggcaacccatatacattcctcttgtctgtaactgccaaaggttgggtttatgtcacttcagttccactcaagcattgaaaaggttctcatggagtctggggtgtgcccagtgaaaagatggggactttttcattatccacagacctctctatacctgctttgcaaaaattataatggagtaactatttttaaagcttatttttcaattcataagaaaaagacatttattttcaatcaaatggatgatgtctcttatcccttatccctcaatgtttgcttgaattttgtttgttccctatacctactccctaattctttagttccttcctgctcaggtcccttcatttgtactttggagtttttctcatgtaaatttgtataatggaaaatattgttcagtttggatagaaagcatggagaaataaataaaaaaagatagctgaaaatcaaattgaagaaatttatttctgtgtaaagttatttaaaaactctgtattatatttaaagaaaaaagcccaaccccccaaaaagtgctatgtaattgatgtgaatatgcgaatactgctataataaagattgactgcatggagaaa

SEQ ID NO: 6 - protein, 804 aa, Mus musculus
MAALSGGGGSSSGGGGGGGGGGGGGDGGGGAEQGQALFNGDMEPEAGAGAAASSAADPAIPEEVWNIKQMIKLTQEHIEALLDKFGGEHNPPSIYLEAYEEYTSKLDALQQREQQLLESLVFQTPTDASRNNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPVPQEEASFPETALPSGSSSAPPSDSTGPQILTSPSPSKSIPIPQPFRPADEDHRNQFGQRDRSSSAPNVHINTIEPVNIDEKFPEVELQDQRDLIRDQGFRGDGAPLNQLMRCLRKYQSRTPSPLLHSVPSEIVFDFEPGPVFRGSTTGLSATPPASLPGSLTNVKALQKSPGPQRERKSSSSSSSEDRSRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 7 - DNA, 2232 bp, Oryctolagus cuniculus
  source 1..2232: mol_type = genomic DNA
atggggaatgtgtggaatatcaaacaaatgattaagttgacacaggagcatatagaggccctattggacaaatttggtggggagcataatccaccatcaatatatctggaggcctacgaagaatacaccagcaagctagatgccctccaacaaagagaacagcagttattggaatccctagtttttcaaaatcccacagatgtgtcacggagcaaccccaagtcaccacaaaaacctattgttagagtcttcctgcccaacaaacagaggacagtggtacctgcaagatgtggagttacggttcgagacagtctaaagaaagcgctgatgatgagaggtctgatcccagaatgctgtgctgtttacagaattcaggatggagagaagaagccaattggctgggacactgatatttcctggctcactggagaagagctgcatgtggaagtgttagagaatgtcccactcaccacacataactttgtacggaaaacttttttcaccttagcattttgtgacttctgtagaaagctgcttttccagggtttccgctgtcaaacatgtggctacaaatttcaccagcgttgtagtacggaagttccactgatgtgtgttaattatgaccaacttgatttgctgtttgtctccaagttctttgaacaccacccagtaccacaggaggaggcctccttagcagagactgccctcacatctgggtcatcgccttccgcacctccctcagactctattgggcaccaaattctcaccagtccgtccccttcaaaatccattccgattccacagtccttccgaccagcagatgaagatcatcgaaatcagtttgggcaacgagaccggtcttcatcagcgcctaatgttcacattaacacaatagaacctgtcaatattgatgaaaaattcccagaagtggaattacaggatcaaagggacttgattagagaccaagggtttcgtggtgatggagcccctttgaaccagctgatgcgctgtcttcggaaataccaatcccggactcccagtcccctcctaccttctgtccccagtgacatagtgtttgattttgagcctggcccagtgttcagaggatcgaccacgggtttgtctgccactccccctgcctcattacctggctcactcactagtgtgaaagctgtacagagatccccaggacctcagcgagagaggaagtcgtcttcctcctcagaagacaggaatcgaatgaaaactcttggtagacgggattcaagtgatgattgggagattcctgatgggcagatcaccgtgggacagagaattggatctggatcatttggaaccgtctacaagggaaaatggcacggtgatgtggcagtaaaaatgttgaatgtgacagcacctacacctcagcagttacaggccttcaaaaatgaagtaggagtactcaggaaaacacgacatgtgaatatcctacttttcatgggctattccacaaagccacagctggctattgttacccagtggtgtgagggctccagtttatatcaccatctccacatcattgagaccaaattcgagatgatcaaacttatagatattgcacggcagactgcacagggcatggattacttacacgccaagtcaatcatccacagagacctcaagagtaataatatatttcttcatgaagacctcacagtaaaaataggtgattttggtctagccacagtgaaatctcgatggagtgggtcccatcagtttgaacaattgtctggatccattttgtggatggcaccagaagtaatcagaatgcaagacaaaaacccatatagctttcagtcagatgtatatgcatttgggattgttctgtatgaattgatgactgggcagttaccttactcaaacatcaacaacagggaccagatcatttttatggtgggacgtggctacctgtctccagacctcagtaaggtacggagtaactgtccgaaagccatgaagagattaatggcagagtgcctcaaaaagaaaagagatgagagaccactctttccccaaattctcgcctccattgagctgctggcccgctcattgccaaaaatccaccgcagtgcatcagaaccctccttgaatcgggctggtttccagacagaggattttagtctatatgcttgtgcttctccaaaaacacccatccaggcagggggatatggagaatttgcagccttcaagtag

SEQ ID NO: 8 - protein, 743 aa, Oryctolagus cuniculus
MGNVWNIKQMIKLTQEHIEALLDKFGGEHNPPSIYLEAYEEYTSKLDALQQREQQLLESLVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPVPQEEASLAETALTSGSSPSAPPSDSIGHQILTSPSPSKSIPIPQSFRPADEDHRNQFGQRDRSSSAPNVHINTIEPVNIDEKFPEVELQDQRDLIRDQGFRGDGAPLNQLMRCLRKYQSRTPSPLLPSVPSDIVFDFEPGPVFRGSTTGLSATPPASLPGSLTSVKAVQRSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 9 - DNA, 2186 bp, Cavia porcellus
  source 1..2186: mol_type = genomic DNA
atggcggcgctcagcggcggcggtggcgcggagcagggccaggctctgttcaacggggacatggagctcgaggccggcgccggcgccgcagcctcttcggctgcagaccctgccattcccgaggaggtatggaatatcaaacaaatgattaagttgacgcaggaacacatagaggccctattggacaaatttggtggagagcataatccaccatcaatatacctggaggcctatgaagaatacaccagcaaactagatgccctccaacaaagagaacagcagttactggaatccctcgggaatggaactgatttttctgtttctagctctgcatcactggacaccgttacatcttcttcttcttctagcctttcagtactaccttcatctctttcagtttttcaaaatcctacagatgtgtcacggagcaaccccaaatcaccacaaaaacctattgttagagtcttcctgcccaacaaacagaggacagtggtacctgcaaggtgtggagttacagtccgagacagtctgaagaaagcactcatgatgagaggtcttatcccagagtgctgtgctgtgtacagaattcaggatggagaaaagaaaccaattggctgggacactgacatttcctggcttactggggaagaattacatgtagaagtattggagaatgttccacttacaacacacaattttgtatgtatctttatattttttttgctgtttgtctccaagttctttgaacaccacccaataccacaggaggaggcttccttagcagagaccacccttacatctggatcatccccttctgcacccccctcagagtccattgggcccccaattctcaccagcccatctccttcaaaatccattccaattccacagcctttccggccaggagaggaagatcatcgaaatcaatttgggcagcgagaccggtcctcatctgctcccaatgtgcatataaacacaatagaacctgtcaatattgatgatttgattagagaccaagggtttcgtagtgatggaggatcaactacaggtttgtctgccaccccacctgcctcattacctggctcactcactaatgtgaaagccttacagaaatctccaggacctcagcgagaaaggaagtcatcttcatcctcagaagacagaaatcgaatgaaaacgcttggtagacgggactcaagtgatgattgggagattcctgatgggcagattacagtgggacaaagaattggatctgggtcatttggaacagtctacaaggggaagtggcatggtgacgtggcagtgaaaatgttgaatgtgacagcacccacacctcaacagttacaggccttcaaaaatgaagtaggagtactcaggaaaacacgacatgtgaatatcctactcttcatgggctattccacaaagccacagctagctattgttacccagtggtgtgagggctccagcttataccaccatctccacatcatcgagaccaaatttgagatgatcaaacttatagatattgcacgacagactgcccagggcatggattacttacacgccaagtcaatcatccacagagacctcaagagtaataatatatttcttcacgaagacctcacggttaaaataggtgattttggtctagccacagtgaaatctcgatggagtgggtcccatcagtttgaacagttgtctggatccattttgtggatggcaccagaagtaatcagaatgcgagataaaaacccatacagttttcagtccgatgtatatgcatttgggattgttctatatgaattgatgactgggcagttaccctattcaaatatcaacaacagggaccagataatttttatggtgggacgaggatatctatctccagatctcagcaaggtacggagtaactgtccaaaagccatgaagaggttaatggcggagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctctatgcttgtgcttctccaaaaacacccatccaggcagggggatatggtgcgtttcctgtccactgatgcaaattaaatgagtgagaaataaa

SEQ ID NO: 10 - protein, 719 aa, Cavia porcellus
MAALSGGGGAEQGQALFNGDMELEAGAGAAASSAADPAIPEEVWNIKQMIKLTQEHIEALLDKFGGEHNPPSIYLEAYEEYTSKLDALQQREQQLLESLGNGTDFSVSSSASLDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVCIFIFFLLFVSKFFEHHPIPQEEASLAETTLTSGSSPSAPPSESIGPPILTSPSPSKSIPIPQPFRPGEEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMRDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 11 - DNA, 3229 bp, Canis lupus
  source 1..3229: mol_type = genomic DNA
gtaatgctggattttcatggaataagtttgacctgtgctgcagtggcctccagcaaggtacccgcaagatgtggagttacagtccgggacagtctaaagaaagctctgatgatgagaggtctaatcccagagtgctgtgctgtttacagaattcaggatggagagaagaaaccgattggctgggacactgatatttcctggctcactggagaggaattgcatgtagaagtgttggaaaatgttccgcttaccacacacaactttgtacggaaaacttttttcaccttagcattttgtgacttttgtcgaaagctgcttttccagggttttcgctgtcaaacatgtggttataaatttcaccagcgttgtagtacagaggttccactgatgtgtgttaattatgaccaacttgatttgctgtttgtctccaagttctttgaacaccacccaataccacaggaggaggcctccatagcagagactgcccttacgtctggatcatccccttctgctcccccctccgattctcctgggcccccaattctgaccagtccgtctccttcaaaatccattccaattccacagcctttccgaccagcagatgaagatcatcgaaatcagtttggacaacgagaccggtcctcatcagctccaaatgtgcatataaacacaatagaacccgtcaacattgatgacttgattagagaccaagggtttcgtagtgatggaggatcaaccacaggtttgtctgccaccccccctgcctcattgcctggctcactcactaatgtaaaagcattacagaaatctccaggacctcagcgggaaagaaaatcatcttcatcctcagaagataggaatcgaatgaaaacacttggtagacgggattcaagtgatgattgggagatacctgatgggcagatcacagtgggacagagaattggatccgggtcatttgggacagtctacaagggaaagtggcatggtgacgtggcagtgaaaatgttgaatgtgacagcacccacacctcagcagttacaggccttcaaaaatgaagtaggagtactcaggaaaactcgacatgtgaatatcctactctttatgggctattcaacaaagccccaactggctattgttacccagtggtgtgagggctccagcttatatcaccatctccacatcattgagaccaaatttgagatgataaagcttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtgatccgaatgcaagacaaaaacccatatagcttccagtcagatgtatacgcatttgggattgttctatatgaattgatgacagggcagttaccttattcaaacatcaacaacagggaccagataatttttatggtgggacgaggatatctttctccagatctcagtaaggtacggagtaactgtccaaaagccatgaagagattgatggcagagtgcctaaaaaagaaaagagatgagaggccactctttccccaaattctcgcctctattgagctgctggcccgctcattgccaaaaattcaccgcagtgcatcagaaccctccttgaatcgggctggcttccaaacagaggattttagtctctatgcttgcgcttctccaaaaacacccatccaggcagggggatacggagaatttgcagccttcaagtagccacaccatcatggcaacaactactcttatttcttaagtcttgtgttcgtacaatttgttaacatcaaaacacagttctgttcctcaaatctttttttaaagatacagaattttcaatgcataagctggtgtggaacagaatggaatttcccatccaacaaaagagggaagaatgttttaggaaccagaattctctgctgccagtgtttcttcttcaacacaaataccacgtgcatacaagtctgcccactcccaggaaggaagaggagagcctgagttctgaccttttgatggtcaggcatgatggaaagaaactgctgctacagcttgggagattggctgtggagagcctgcccgtcagctctgcccttctaaccgccagatgagtgtgtggctggtcacctgacagggcagctgcaatcgccaagcatcgttctctttcctgtcctgggattttgtcgtggagctctttccccctagtcaccaccggttcatttctgagggatggaacaaaaatgcagcatggcctttctgtgtggtgcatgtccggtctttgacaaatttttatcaagtgaagctcttgtatttaaatggagaatgagaggcgagggggggggatcacgttttggtgtaggggcaaagggaatgctgcatctttttcctgacccactgggtttctggcctttgtttccttgctcactgagggtgtctgcctataaccacgcaggctggaaagtgctggcacacattgccttctcttctcactgggtccagcaatgaagacaagtgttggggatttttttttttgccctccacaatgtagcaagttctcaggaaaatacagttaatatcttcctcctaagctcttccagtcatcaagtacttatgtggctactttgtccagggcacaaaatgccatggcggtatccaattaaaagcctacaaaactgcttgataacagttttgaatgtgtgagacatttatgtaatttaaatgtaaggtacaagttttaatttctgagtttctctattatatttttattaaaaagaaaataattttcagatttaattgaattggaataaaataatacttcccaccagaattatatatcctggaaaattgtatttttgttatataaacaacttttaaagaaagatcattatccttttctctacctaaatatggggagtcttagcataatgacagatatttataatttttaaattaatggtacttgctggatccacactaacatctttgctaatatctcatgttttcctccaacttactcctacactacatcctccatcctctttccagtcttttatctagaatatgcaacctaaaataaaaatggtggtgtctccattca

SEQ ID NO: 12 - protein, 617 aa, Canis lupus
MLDFHGISLTCAAVASSKVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASIAETALTSGSSPSAPPSDSPGPPILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 13 - DNA, 1889 bp, Canis lupus
  source 1..1889: mol_type = genomic DNA
ggaatatcaaacaaatgattaagttgacacaggaacatatagaagccctattggacaagtttggtggggagcataatccaccatcaatatatctggaggcctatgaagaatacaccagcaaactagatgccctccaacagcgagaacaacagttattggaatccctggggaatggaactgatttttctgtttctagctctgcatcaacggacaccgttacatcttcttcctcttctagcctttcagtgctaccttcatctctttcagtttttcaaaatcccacagatatatcacggagcaatcccaagtcaccacaaaaacctatcgttagagtcttcctgcccaataaacagaggacggtggtacccgcaagatgtggagttacagtccgggacagtctaaagaaagctctgatgatgagaggtctaatcccagagtgctgtgctgtttacagaattcaggatggagagaagaaaccgattggctgggacactgatatttcctggctcactggagaggaattgcatgtagaagtgttggaaaatgttccgcttaccacacacaactttgtacggaaaacttttttcaccttagcattttgtgacttttgtcgaaagctgcttttccagggttttcgctgtcaaacatgtggttataaatttcaccagcgttgtagtacagaggttccactgatgtgtgttaattatgaccaacttgatttgctgtttgtctccaagttctttgaacaccacccaataccacaggaggaggcctccatagcagagactgcccttacgtctggatcatccccttctgctcccccctccgattctcctgggcccccaattctgaccagtccgtctccttcaaaatccattccaattccacagcctttccgaccagcagatgaagatcatcgaaatcagtttggacaacgagaccggtcctcatcagctccaaatgtgcatataaacacaatagaacccgtcaacattgatgacttgattagagaccaagggtttcgtagtgatggaggatcaaccacaggtttgtctgccaccccccctgcctcattgcctggctcactcactaatgtaaaagcattacagaaatctccaggacctcagcgggaaagaaaatcatcttcatcctcagaagataggaatcgaatgaaaacacttggtagacgggattcaagtgatgattgggagatacctgatgggcagatcacagtgggacagagaattggatccgggtcatttgggacagtctacaagggaaagtggcatggtgacgtggcagtgaaaatgttgaatgtgacagcacccacacctcagcagttacaggccttcaaaaatgaagtaggagtactcaggaaaactcgacatgtgaatatcctactctttatgggctattcaacaaagccccaactggctattgttacccagtggtgtgagggctccagcttatatcaccatctccacatcattgagaccaaatttgagatgataaagcttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtgatccgaatgcaagacaaaaacccatatagcttccagtcagatgtatacgcatttgggattgttctatatgaattgatgacagggcagttaccttattcaaacatcaacaacagggaccagctcagatcatgatcacggtgtcatgagatcaagccccac

SEQ ID NO: 14 - protein, 615 aa, Canis lupus
MIKLTQEHIEALLDKFGGEHNPPSIYLEAYEEYTSKLDALQQREQQLLESLGNGTDFSVSSSASTDTVTSSSSSSLSVLPSSLSVFQNPTDISRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASIAETALTSGSSPSAPPSDSPGPPILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQLRS

SEQ ID NO: 15 - DNA, 3521 bp, Felis catus
  variation 630..649: note = n is a, c, g, or t
  source 1..3521: mol_type = genomic DNA
atgcctaacctcagtctctgccaccacggccaatttgctcatgtgcccactgtgtcggcactgggatattttgtgatttgccttggccattgtccactgtccttgacattgcctgaaggagaaccactgatgctaatgttgaaggtgacctttgcaggctctccactactcataccaaagatgcggccccctgataatcccagagccactgtctgcacatgggcaaaacaggctacattctgtgcagactggggagaaaggttccaagaacacagtgccatagttttgggcagagagtttcaacacagcatagtgtctatggcagtatctggatttggccgggggaagtgcccaagaggagacagtcaggctgtgtcctacggccaaggacctgcacttatttttgcatgcagtggtttagcacagggaagagaacgaagtaggaaatcggagccatggaaacggcagagcggaggaaacgtgcacgcgcgagggtgggcacgaaaggaaagaaccctccccagaagactgcgcgagggcgctcctaggattacgtcacgcaccccgcgaaaactgaaatgtactgtgtgtggtcttttaattgaactatcttccttatgtgcacttaannnnnnnnnnnnnnnnnnnngcggcggcggcggtggcgcggagcagggccaggctctgttcaacggggacatggagcccgaagccggcgccgcggcctcttcggctgcggaccctgccattcccgaggaggtgtggaatatcaaacaaatgattaagttgacacaggaacatatagaggccctattggacaaatttggtggggagcataatccaccatcaatatatctagaggcctatgaagaatacaccagcaagctagatgccctccaacagagagaacaacagttattggaatccctggggaatggaactgatttttctgtttctagctctgcatcaacagacaccgttacatcttcctcctcttctagcctttcagtgctaccttcatctctttcagtttttcaaaaccccacagatgtgtcacggagcaatcccaagtcaccacagaaacctatcgttagagtcttcctgcctaataaacagaggacagtggtacctgcaagatgtggagttacagtccgggacagtctaaagaaagctctgatgatgagaggtctaatccctgagtgctgtgctgtttacagaattcaggatggagagaagaaaccaattggctgggacactgatatctcctggctcaccggagaggaattgcatgtagaagtgttggaaaatgttccacttacaactcacaactttgtatgtacggaaaacgttttcaccttagcattttgtgacttttgtcgaaagctgcttttccaaggttttcgctgtcaaacgtgtggttataaatttcaccagcgttgtagtacagaggttccactgatgtgtgttaattatgaccaacttgatttgctgtttgtctccaagttctttgaacaccacccaataccacaggaggaggcctccatagcagagactgccctaacgtctggatcgtccccttctgcccccccctccgattctactgggccccaaattctcaccagtccgtctccttcaaaatccattccaattccacagcctttccgaccagcagatgaagatcatcgaaatcaatttggacagcgagaccggtcctcatcagctccaaatgtgcatataaatacaatagaacctgtcaatattgatgacttgattagagaccaggggtttcgtagtgatggaggatcaaccacaggcttgtctgccaccccccctgcctcattgccgggctctctcactaatgtaaaagcattacagaaatctccagggcctcagcgggaaaggaaatcttcttcatcctcagaagataggaatcgaatgaaaacacttggtagaagggattcaagtgatgattgggagattcctgatgggcagatcacagtgggacagagaattggatccgggtcatttgggacagtctacaagggaaagtggcatggtgatgtggcagtgaaaatgttgaatgtgacagcacccacacctcagcagttacaggccttcaaaaatgaagtaggagtactcaggaaaactcggcatgtgaacatcctgctcttcatgggctattcaacaaagccccagctggctattgtcacccagtggtgtgagggctccagcttataccaccatctccacatcatcgagaccaaattcgagatgatcaagctgatagatattgctcggcagactgcgcagggcatggattacttacacgccaagtcaatcatccacagagacctcaagagtaataatatttttcttcacgaagacctcacagtaaaaataggtgattttggtctagccacagtgaaatctcgatggagtgggtcccatcagtttgaacagttgtctggatccattttgtggatggcaccagaagtaattcgaatgcaagataaaaacccatatagctttcagtcagatgtatatgcatttgggattgttctatatgaattgatgactggacagttaccttattcaaacatcaacaacagggaccagataatttttatggtgggacgaggatatctttctccagatctcagtaaggtacgaagtaactgtccaaaagccatgaagagattgatggcagagtgcctaaaaaagaaaagagatgagaggccactgtttccccaaattcttgcctctattgagctgctggcccgctcattgccaaaaattcaccgcagtgcatcagaaccctccttgaatcgggctggcttccagacagaggattttagtctctatgcttgtgcttctccaaaaacacccatccaggcagggggatatggtgcgtttcccgtccactgagataagttagatgagtgcgcgagtgcagggggccggggccaaggaggtggaaatgtgcgtgcttctgtactaagttggatagcatcttcttttttaaaaaaagatgaaccaaagaatgtgtatgtttttaaagactagatataattatttcctgatctaaaatgtatacttagctttggattttcaatatccaagggttttcaaaatgcacagacattgctgaacatttgcagtacctcttctggaggctttacttcctgttacaaattggttttgtttactggcttatcctaattattaaacttcaattaaacttttctcctgcaccttttgttatgagctatcacatgtcccttagggactcgcaagagcagtactgcccccgtgtacgggcttgcaggtagaaaggggatgacgggttttaacacctgtgtgaggcaaggcagtccgaacagatctcatttaggaagccacgagagttgaataagttatttttattcttagtattttttctgtaactactttttattataacttggaaaatatggatgtcctttatacaccttagcaatagactgaatttctttttataaatt

SEQ ID NO: 16 - protein, 974 aa, Felis catus
  VARIANT 210..217: note = X can be any naturally occurring amino acid
MPNLSLCHHGQFAHVPTVSALGYFVICLGHCPLSLTLPEGEPLMLMLKVTFAGSPLLIPKMRPPDNPRATVCTWAKQATFCADWGERFQEHSAIVLGREFQHSIVSMAVSGFGRGKCPRGDSQAVSYGQGPALIFACSGLAQGRERSRKSEPWKRQSGGNVHARGWARKERTLPRRLREGAPRITSRTPRKLKCTVCGLLIELSSLCALXXXXXXXXGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVCTENVFTLAFCDFCRKLLFQGFRCQTCGYKFHQRCSTEVPLMCVNYDQLDLLFVSKFFEHHPIPQEEASIAETALTSGSSPSAPPSDSTGPQILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 17 - DNA, 3853 bp, Bos taurus
  source 1..3853: mol_type = genomic DNA
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaaattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatggtacgtttcctgttcactgaaacaaaccgagtgagtgacagcatgtaggagggtagggacaaaagaaagtgaacaaatgtttgcttatatatttgttaaattgaataggattttctttttctttaaaggtgaacaagagaacatgtgtgtttttaaagtttggatatagttttcttcccagtctaaaacccatagttagcattacattttcaacatcgaatttttttttaattcatagacattgctgaaaatttataataccttttccagaggctttacttcccattccaagtttgttttgtttacttggttagtctaatcattaaactttaaactttccccacctaccttttgctgttagctatcccgcatccattaggggctccaagaacagcactgtctgcgtgtgtgtgttggcaggtgggaagctgatggtaagttaggctgtgttagtgaaggtaaactgaccaggtctaattaggagtcactagaattgaataagcttatttttattaatattttttcttataactatttctttttgtaataatttagaaaatataattgttctttattcccttacagcagtataaattattggtgcaggtaaccaaagatattactgaggagtggcatgtttgacatgagtgacatggtttaactttggatttttagttaatatttctttatatattaaggatgtcttacacattatagaagtcaaatttactgacaaaggtattgcctcctcttcctccccaaaaacacagcaaaattctctgggaactcgtagcattgttggttttcttttggatgactatggttgccaaacaaccaagtaattgattttttttaaattattattgctttagattatactcacctctcatgatgcctgttagcaatcacctttatccatgtgtcttgtaaaatatctttcctccttatattctttgcccaacaagagtctacttgttatgaatgagtactattttctttttttgattccccagtataattagtatgtttagtgctttctaggacttccactttcttatgttaaaaaaaaaaacaaactaatgtggcagtcagtatattcttactgtgaatcagagtctttactgggaatcaaagtgaaagaagcagctgttctgacttcagagtcagcctagggaccaaaaccagcctcttaaatacaccttcatttattcagtttggatttgtgatgattttcattatagctgacagttcaaggttattcagtggcacacagatagcatctgcataaatgcctttcttcttgaaaataaaggagaaaattgggaagactttacaccaatagtttagtctttaagtaccacagataacacacaccataaat

SEQ ID NO: 18 - protein, 764 aa, Bos taurus
AALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGTFPVH

SEQ ID NO: 19 - DNA, 4936 bp, Bos taurus
  source 1..4936: mol_type = genomic DNA
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatgaagcagatttggctcttacatcaaataaaaatagagtagaagttgggatttagagatttcctgacatgcaagaaggaataagcaagaaaaaaaggtttgttttccccaaatcatatctattgtcttttacttctattttttcttaaattttttgtgatttcagagacatgtagagttttattgatacctaaactatgagttcttttttttttttttttttcattattttgatttttttggccaagaggcatatgggatcttagcttgagaaagcaacaattttcttgatgtcattttgggtgagggcacatattgctgtgaacagtgtggtgatagccaccagggaccaaactcacacccgctgcattgaaaggtgaagtcttaaacactggaccagcagagaaattcctactctatgagttctttttgtcatcccctccccgcaccctccacccccaacctaaagtctgatgatgaaatcaacaactattccattagaagcagtagattctggtagcatgatctttagtttgttagtaagattttgtgctttgtggggttgtgtcgttttaaggctaatatttaagtttgtcaaatagaatgctgttcagattgtaaaaatgagtaataaacatctgaagttttttttaagttatttttaacatggtatatacagttgagcttagagtttatcattttctgatattctcttacttagtagatgaattctagccattttttataaagatttctgttaagcaaatcctgttttcacatgggcttcctttaagggattttagattctgctggatatggtgactgctcataagactgttgaaaattacttttaagatgtattagaatacttctgaaaaaaaatagcaaccttaaaaccataagcaaaagtagtaagggtgtttatacatttctagagtccctgtttaggtaatagcctcctatgattgtactttaaatgttttgctctccaaggttttagtaacttggctttttttctaatcagtgccaaactcccccagtttttttaactttaaatatgaggtaataaatcttttacccttccttgatcttttgacttataataccttggtcagttgtttcttaaaaggaatccttaaatggaaagagacaatatcactgtctgcagttctgattagtagttttattcagaatggaaaaacagattattcatttttgaaaattgttcaggggtatgttcattgttaggaccttggactttggagtcagtgcctagctatgcattccaggtctgccattttctggctgtgaaattttggacaagttacttaaccactttaaaccccagctttaagaagtaaattaaccccagtaaattaagaagtaatagcagccacttcgtagagttgttatgaggctcagatgcagtgcaaatgtgtataaagtattcagggagtcacctggtatactataatagacactagaatagttgccaatattatcagcatacaatctgaggattctgtcagccaatcattagcaatctgttgtttgttgggacatgccagtgttctccagttgaaatcagtagcaatctaaaaatggatagattattcctcatttaaatagtgtgttcatataagtgattgcttggatccttatcagaagttgctgttactgaaaaatgataaggctgactaaattgtgatagttgtcagttactaaccaactcccagaaatgaataagaggaacctatctctagttcctagtagaaggtatggacaaaatagtaggtgaaaaataatgtcttgaacccccaaattaagtaagctttaaagagtacaatacctcaaagggtctttgcggtttaaaatttgtatgctgagaatgatgttcattgacatgtgcctatatgtaattttttgatagtttaaaaggtgaaatgaactacagatgggagaggtctgaattttcttgccttcagtcaaatgtgtaatgtggacatattatttgacctgtgaattttatcttttaaaaaagattaattcctgcttcttccttcctaatagttgcattataataatgaaaatgagttgataatttggggggaaagtattctacaaatcaaccttattattttaccattggtttctgagaaattttgttcatttgaaccgtttatagcttgattagaatcatagcatgtaaaacccaactgagggattatctgcagacttaatgtagtattatgtaagttgtcttctttcatttcgaccttttttgcttttgttgttgctagatctgtagtatgtagctagtcacctttcagcgaggtttcagcgaggcttttctgtgtctctaggttatttgagataacttttttaaaattagctcttgtcctcc

SEQ ID NO: 20 - protein, 797 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYEADLALTSNKNRVEVGI

SEQ ID NO: 21 - DNA, 4154 bp, Bos taurus
  source 1..4154: mol_type = genomic DNA
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatggctgagcacattgtccatcacccacaagtggctggttctcatcgcagaatctacgtagggaatcgggcgtgaaattcacttaagagatagagcagaggaagtgttctgtttacaggaatggagatgagagttatgagtaagttgcttagtcagttggctttgttttgaaaattattgtgttatatttgtgttaacctacttgtgttttgacagtatatgtcacataggaagaaacctcagactagcataataacaaagctcagactaggcacagatgtacacagaatggaccaaaatgggatgggggaaggtatgggaataagtctaggggtagggaaaaattgatgtgagggtgggaaataaactgtaattacctgaaataaaatgtaagagtgcaataagtgtgctttttattctaagctgtgaatgggttttttaaaaaaagcattccttcccaatgcatttgcctatgttccatagctgattaaaaccagctatataaacatatgcctttttattcatgttaattaccaatataaatggctaacctttacgtcttatttatcttcatgttatgttagtttacatacagggatgtgtgtgtgtgtgtatgctataaattttccctccttcgtttaaaaacgcgtttgttggatcctctctgtttccttaggccatgccacagctcatagtctcagcttggccttcctgtcacctgatctgaaggactatcacagtgacgtagctcgttcattggttgtacacactctaacccttttccttgctcagcaattactgtgtcttctaaaacaggagtgtacaaccatgagattgcaattaattgtttgacatatgtccctttgaattctatttattagttatgattgattgctctttggtttggaccaagaaaaacgaaatcccacctccccaccttttcacttatttcttactttgaggacaattctgtaagagagaggaaagggaactccttcatgttttaactgcagcaagttaatggccctggtttacaccaaacattatggtgattcacattcacattcctctcctctcttgctgccagaggtttgggttttgttcagttctgctcaagcactgaaaaagttttcatggagtctggagagtgcccagtgaaaagatggtttttaattgtccacagacctttctgttcctgctttgcaaaaattacaaaggagtaactatttttaaagcttatttttcaattcataaaaaagacatttattttcagtcagatgatgtctccttgtcccttaatcctcaatgtttgcttgaatcttttttttttttctgattttctcccatccccacttcttgatacttcttgagttctctttcctgctcaggtcctttcatttgtactttggagttttttctcatgtaaatttgtacaatggaaaatattgttcagtttggatagaacgcatggagaattaaataaaaaagatagctgaaattcagattgaaatttatttgtgtaaagttatttaaaaactctgtactatataaaaggcaaaaaaagttctatgtacttgatgtgaatatgcgaatactgctataataaagattgactgcatgga

SEQ ID NO: 22 - protein, 781 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYG

SEQ ID NO: 23 - DNA, 7914 bp, Bos taurus
  source 1..7914: mol_type = genomic DNA
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaaattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatggagaatttgcagccttcaagtagccacaccatcatgacagcatctactcttatttcttaagtcttgtgttcgtacaatttgttaacatcaaaacacagttctgttcctcaactctttttaaagttaaaatttttcagtgcataagctggtgtggaacagaaggaaatttcccatccaacaaaagagggaagaatgttttaggaaccagaattctctgctgccagtgtttcttcttcaacacaaatatcacaagtctgcccactcccaggaagaaagaggagagaccctgagttctgaccttttgatggtcaggcatgatggaaagaaactgctgctacagcttgggagatttgctctgggaagtctgccagtcaactttgcccttctaaccaccagatcaatatgtggctgatcatctgatggggcagttgcaatcaccaagccttgttctctttcctgttctgggattgtgttgtggaacccttttccctagccaccaccagttcatttctgagggatggaacaaaaatgcagcatgcccttcctgtgtggtgcatgttcagtccttgacaaatttttaccaaaatgaagctactttatttaaaaggagggtgagaggtgaggaggtcactttgggtgtggcggaaagggaatgctgcatctttttcctgggctgctggggctctggccttggcttgccagccggaagcgctggcacgcatcgccttcttttcccattgggtccagcaatgaagacgagtgtttggggttttttttttctccaccatgtagcaagttctcaggaaaatacaattgatatcttcctcctaagctcttccaatcagtcaccaagtacttatgtggttactttgtccagggcacaaaatgcctgtatctaattaaaagcctacaaaactgcttgataacagttttgaatgtgagacatttatgtaatttaaatgtaaggtacaagttttaatttctgagtttcttctattatatttttattaaaaaaagaaaataattttcagattgaattggagtaaaataatattacttcccactagaattatatatcctggaaaattgtatttttgttacataagcagcttttaaagaaagatcattacccttttctctacataaatatatggggagtcttagcctaatgacaaatatttataatttttaaattaatggtacttgctggatccatactaacatctttactaatacctcattgtttcttccaacttactcctacactacatcctacatcttcttcctagtcttttatctagaatatgcaacctcaaataaaaatggtggtgtcctcattcattctcctccttccttttttcccaagcctgatcttcaaaaggttggttaatttggcagctgagttcctccccaggcagagaatagaccaattttaggtgtattgggactgagggaggatgtgtaaagattaacatcagtaaagaaccgctgtggagtaattaagaactttgttctttataactggagaatataacctaaccctaacatccctcagcctttactaaagtgtggcgtaaatcacagtagtagcaaagaaagtgactctggatgtgttcctggccagtacctcccttatcatgaatgtagactctctcatcaagatttaggaatataaatcaaatcaaatgtgcccagccaagctatgtagtaagggacttgaacaatattaggcagaacctataaaataaatcagggaattagaaattatttaaagttttcaaattgtaaattgccccggtgtctttcagcctactgccattatttttgctacaatacctacatttcagaggagggcctactgaaaattccatgcaagtggaaaataatcctcaagttattaatgagtttgaaaagcaatgagttcttaagtctttgtgagtagagcaagatcctacaaaattcagaaatagtaaaaatggattcatgctgatttgaagagcatctgtgtgcataatataatgctgcatctcttttaaaagcagtctatttttctttttaaatttgtccccatagatgcttttgaacatgaacatgcttatgttaccttttccgaggttgggaagagccaggagctctcaggcagggccccctccctcagctgggcaggagctgctcaggaggagctagttatagaggaagcttagcgttggcattttcaaaattcaaggtgataacgctttcttcttcctttctgttttagaatagattgctgtctgatttgaaaaagggaaatagatttgatctcaaatgaatctgtgcccagaagccaggctcagggtattcagagatttgtatagtgccctcaaaaaataacaaaattttagctttccttttttcttcttttctccatcaaattcttttttctctagtttacaaatgacatggaaaaggaatttcccctgagttttgtatgcctttttttttttggcttagactatagataggcgtgttgagctcctaagaaaatacaaggaggaactctttgttgtgcagagcactttatgagtagtttgtgtggataatatgtgactgcttccctgacgagcttgtgaggctgtacttatgtctttcctgtaaggcagcttcagtgccttctgtagtgtatataaggaaagattacgccttctgaaaaatctcagagcaaccataagattattttaaaatatgtagtatgactgatggactttttcatcattaaattagtctagcatctaaacttttaccactgaaataatattgaccaaaaagcaatttataaaaggtatttgtgaatagaaaatacaatgtgatcatttgtacttatgtgcaccttaaaagaggaattctgtctagctgtcaaattctggttccttaacatccagtccttgattgtgattgagatctggtaggacgtgctggggcacgctagcagataaaatcccgtatactttaggatagatgttacatttatgtcagtgttggcaaagagcattgtgtagtaataaagaattcaagacttcagcaatgtcaacctgaaactttgtaaatatttcctagattgttatttgatgcagtcacagctctttatcacacaatgttgtctttccctcatcaggcaattttagaactgctgcacacccctcctcagatctcacctgcccctcctgtacattcacctctccagccttgtgcacacctcatttagctttagtttgaaacacattgcagggttcaggtgacctcttcaaaaactacctcctcagaatgaggtaatgaatagttatttattttaaaatatgaaaagtcaggagctctagaatatgaagatgatctaagattttaacttttatgtatacttgttgagcactctccttttgtcctaaagggcattatacatttaagcagtaatactgaaaaatgtagctcagagtaactgaatgttgttgaaagtggtgccagaatctgttttaggggtacgtatcagaatcttaatcttaaatcggttacatgaaattaaatagttaatggtaacacttgactaacagatataattttaattttcggtaggcttttagcaagacagtaagtacatcttcataatgagttagccacagcttcatcacatgcacagattttcctgttgagagactgcccagttaagagggtagaatgatgaaccatttttcaggattctcttctttgtccaaactggcattgtgagtgctagaatatcagcactttcaaactagtgattccaactattaggctattaaaaagcaaaacaaaccaaacaaaccatagccagacatgggaagtttactatgagtataaacagcaaatagcttacaggtcatacattgaaatggtgtaggtaaggcgttagaaaaataccttgacaatttgccaaatgatcttactgtgccttcatgatgcaataaaaaaaaaaaaaatttagcataaatcagtgatttgtgaagagagcagccaccctggtctaactcagctgtgttaatattttttagcgtgcaatttagactgcaaagataaatgcactaaagagtttatagccaaaatcacatttaaaaaatgagagaaaacacaggtaaattttcagtgaacaaaattatttttttaaagtacataatccctagtatagtcagatatatttatcacatagagcaaataggttgaaatcacaattcagtgacatttctagagaaactttttctactcccataggttcttcaaagcatggaacttttatataacagaaatgtgtgacggtcattttaaattgctgtagtttggggctgaagtactgtgtgctgggcagcaatcacatgtattaactagtgagaaaggagaaattaagatataggacagaatttgattttcttgttcccagattactgctgccaacctagacactgagtttccagaggctgaaacgtaaacttgcagctcagcaactgttttgcaaagttagtgggactgtcctgcttatgctgttcaaaaatgctctgagggccaggtggggcctccaggggctcctctctgaggggacatcagactagctaacgacctggcgggcggatgtgaaccggacacactccatggtgtgcttcttgtatcggtccctcgccaccctcaagaaaggcttcagcgggttctctagacgtctccactaaggtgtgttactaacagccatgggttgttgagcacccgaggagtgcaatagcatctctgcatgattgtatattggcccgaagagaatgaagtggccagtgtactcatgttccatgttgctagctctggtaaactgaaaatactggtaagatttttgttttatcagtacactagagagtaagctttgttttgttgtttttagataatgttttcacttccatttggaaagacatttaaattgagtttcagtcctaaattttgccagtcatggtaattagcagtttctatcaggtatttttaaggtagaagaggatagaaacataagttctaaaagcttaaggtaaccgtggtttattttaaaatgtttaggggtggttagtctctacctcaaaaaaagtgagtgaatcttttatttcagcattcacaagttcggctgttgtttttgtaatacattttttttttaaccttttgacccccctttacctaagtgtcaatgtagttttattaattactaagtcagtttcattaaaatgtttatttagcagttttgactaattgcaatgattaatatagccagttgtgcatgaggacacagccagtgagtatatctgggttttttttgtgatgctttttttcttaagacttctgtagatttatgaagtactcattgaaaacaactaaaatacgtttattcgtgttaatatggaaaaaaaaaaa

SEQ ID NO: 24 - protein, 766 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 25 - DNA, 4670 bp, Bos taurus
  source 1..4670: mol_type = genomic DNA
ggtgtgtcatagtgcagcagattgaatgcagaagatatgaaaattcagatgtcttctgttaaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatgaagcagatttggctcttacatcaaataaaaatagagtagaagttgggatttagagatttcctgacatgcaagaaggaataagcaagaaaaaaaggtttgttttccccaaatcatatctattgtcttttacttctattttttcttaaattttttgtgatttcagagacatgtagagttttattgatacctaaactatgagttcttttttttttttttttttcattattttgatttttttggccaagaggcatatgggatcttagcttgagaaagcaacaattttcttgatgtcattttgggtgagggcacatattgctgtgaacagtgtggtgatagccaccagggaccaaactcacacccgctgcattgaaaggtgaagtcttaaacactggaccagcagagaaattcctactctatgagttctttttgtcatcccctccccgcaccctccacccccaacctaaagtctgatgatgaaatcaacaactattccattagaagcagtagattctggtagcatgatctttagtttgttagtaagattttgtgctttgtggggttgtgtcgttttaaggctaatatttaagtttgtcaaatagaatgctgttcagattgtaaaaatgagtaataaacatctgaagttttttttaagttatttttaacatggtatatacagttgagcttagagtttatcattttctgatattctcttacttagtagatgaattctagccattttttataaagatttctgttaagcaaatcctgttttcacatgggcttcctttaagggattttagattctgctggatatggtgactgctcataagactgttgaaaattacttttaagatgtattagaatacttctgaaaaaaaatagcaaccttaaaaccataagcaaaagtagtaagggtgtttatacatttctagagtccctgtttaggtaatagcctcctatgattgtactttaaatgttttgctctccaaggttttagtaacttggctttttttctaatcagtgccaaactcccccagtttttttaactttaaatatgaggtaataaatcttttacccttccttgatcttttgacttataataccttggtcagttgtttcttaaaaggaatccttaaatggaaagagacaatatcactgtctgcagttctgattagtagttttattcagaatggaaaaacagattattcatttttgaaaattgttcaggggtatgttcattgttaggaccttggactttggagtcagtgcctagctatgcattccaggtctgccattttctggctgtgaaattttggacaagttacttaaccactttaaaccccagctttaagaagtaaattaaccccagtaaattaagaagtaatagcagccacttcgtagagttgttatgaggctcagatgcagtgcaaatgtgtataaagtattcagggagtcacctggtatactataatagacactagaatagttgccaatattatcagcatacaatctgaggattctgtcagccaatcattagcaatctgttgtttgttgggacatgccagtgttctccagttgaaatcagtagcaatctaaaaatggatagattattcctcatttaaatagtgtgttcatataagtgattgcttggatccttatcagaagttgctgttactgaaaaatgataaggctgactaaattgtgatagttgtcagttactaaccaactcccagaaatgaataagaggaacctatctctagttcctagtagaaggtatggacaaaatagtaggtgaaaaataatgtcttgaacccccaaattaagtaagctttaaagagtacaatacctcaaagggtctttgcggtttaaaatttgtatgctgagaatgatgttcattgacatgtgcctatatgtaattttttgatagtttaaaaggtgaaatgaactacagatgggagaggtctgaattttcttgccttcagtcaaatgtgtaatgtggacatattatttgacctgtgaattttatcttttaaaaaagattaattcctgcttcttccttcctaatagttgcattataataatgaaaatgagttgataatttggggggaaagtattctacaaatcaaccttattattttaccattggtttctgagaaattttgttcatttgaaccgtttatagcttgattagaatcatagcatgtaaaacccaactgagggattatctgcagacttaatgtagtattatgtaagttgtcttctttcatttcgaccttttttgcttttgttgttgctagatctgtagtatgtagctagtcacctttcagcgaggtttcagcgaggcttttctgtgtctctaggttatttgagataacttttttaaaattagctcttgtcctcc

SEQ ID NO: 26 - protein, 761 aa, Bos taurus
MKIQMSSVKVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYEADLALTSNKNRVEVGI

SEQ ID NO: 27 - DNA, 4816 bp, Bos taurus
  source 1..4816: mol_type = genomic DNA
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagagcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatgaagcagatttggctcttacatcaaataaaaatagagtagaagttgggatttagagatttcctgacatgcaagaaggaataagcaagaaaaaaaggtttgttttccccaaatcatatctattgtcttttacttctattttttcttaaattttttgtgatttcagagacatgtagagttttattgatacctaaactatgagttcttttttttttttttttttcattattttgatttttttggccaagaggcatatgggatcttagcttgagaaagcaacaattttcttgatgtcattttgggtgagggcacatattgctgtgaacagtgtggtgatagccaccagggaccaaactcacacccgctgcattgaaaggtgaagtcttaaacactggaccagcagagaaattcctactctatgagttctttttgtcatcccctccccgcaccctccacccccaacctaaagtctgatgatgaaatcaacaactattccattagaagcagtagattctggtagcatgatctttagtttgttagtaagattttgtgctttgtggggttgtgtcgttttaaggctaatatttaagtttgtcaaatagaatgctgttcagattgtaaaaatgagtaataaacatctgaagttttttttaagttatttttaacatggtatatacagttgagcttagagtttatcattttctgatattctcttacttagtagatgaattctagccattttttataaagatttctgttaagcaaatcctgttttcacatgggcttcctttaagggattttagattctgctggatatggtgactgctcataagactgttgaaaattacttttaagatgtattagaatacttctgaaaaaaaatagcaaccttaaaaccataagcaaaagtagtaagggtgtttatacatttctagagtccctgtttaggtaatagcctcctatgattgtactttaaatgttttgctctccaaggttttagtaacttggctttttttctaatcagtgccaaactcccccagtttttttaactttaaatatgaggtaataaatcttttacccttccttgatcttttgacttataataccttggtcagttgtttcttaaaaggaatccttaaatggaaagagacaatatcactgtctgcagttctgattagtagttttattcagaatggaaaaacagattattcatttttgaaaattgttcaggggtatgttcattgttaggaccttggactttggagtcagtgcctagctatgcattccaggtctgccattttctggctgtgaaattttggacaagttacttaaccactttaaaccccagctttaagaagtaaattaaccccagtaaattaagaagtaatagcagccacttcgtagagttgttatgaggctcagatgcagtgcaaatgtgtataaagtattcagggagtcacctggtatactataatagacactagaatagttgccaatattatcagcatacaatctgaggattctgtcagccaatcattagcaatctgttgtttgttgggacatgccagtgttctccagttgaaatcagtagcaatctaaaaatggatagattattcctcatttaaatagtgtgttcatataagtgattgcttggatccttatcagaagttgctgttactgaaaaatgataaggctgactaaattgtgatagttgtcagttactaaccaactcccagaaatgaataagaggaacctatctctagttcctagtagaaggtatggacaaaatagtaggtgaaaaataatgtcttgaacccccaaattaagtaagctttaaagagtacaatacctcaaagggtctttgcggtttaaaatttgtatgctgagaatgatgttcattgacatgtgcctatatgtaattttttgatagtttaaaaggtgaaatgaactacagatgggagaggtctgaattttcttgccttcagtcaaatgtgtaatgtggacatattatttgacctgtgaattttatcttttaaaaaagattaattcctgcttcttccttcctaatagttgcattataataatgaaaatgagttgataatttggggggaaagtattctacaaatcaaccttattattttaccattggtttctgagaaattttgttcatttgaaccgtttatagcttgattagaatcatagcatgtaaaacccaactgagggattatctgcagacttaatgtagtattatgtaagttgtcttctttcatttcgaccttttttgcttttgttgttgctagatctgtagtatgtagctagtcacctttcagcgaggtttcagcgaggcttttctgtgtctctaggttatttgagataacttttttaaaattagctcttgtcctcc

SEQ ID NO: 28 - protein, 757 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLEPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYEADLALTSNKNRVEVGI

SEQ ID NO: 29 - DNA, 2499 bp, Bos taurus
  source 1..2499: mol_type = genomic DNA
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcaggaaaagtatgtttctttagtacattccaggcatttgggattacagtaaaaacaatattctcgcctctattgagctgctggcccgctcattgccaaaaattcaccgcagtgcatcagaa

SEQ ID NO: 30 - protein, 744 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQEKYVSLVHSRHLGLQ

SEQ ID NO: 31 - DNA, 2404 bp, Bos taurus
  source 1..2404: mol_type = genomic DNA
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagatctctcttcccaccatagacacaaaaatttcagatggctacaggtttacatgtaaaaaacagaattataacaaatgatttttat

SEQ ID NO: 32 - protein, 726 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQDLSSHHRHKNFRWLQVYM

SEQ ID NO: 33 - DNA, 2331 bp, Bos taurus
  source 1..2331: mol_type = genomic DNA
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccaggtgctttgtcctccatgggagtgtaataaatgctgtgcaagggcttacttcccatgagagaagtgagtgaccaacagaaggataatttttatggtgggacgaggatatctgtctccagatctcagtaaggtacggagtaactgtccaa

SEQ ID NO: 34 - protein, 681 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQVLCPPWECNKCCARAYFP

SEQ ID NO: 35 - DNA, 2319 bp, Bos taurus
  source 1..2319: mol_type = genomic DNA
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccaggtgctttgtcctccatgggagtgtaataaatgctgtgcaagggcttacttcccatgagagaagtgagtgaccaacagaaggtctgtgcaaggaaaagagacaaagccacggatcagaagcacatggccataactga

SEQ ID NO: 36 - protein, 681 aa, Bos taurus; identical to SEQ ID NO: 34

SEQ ID NO: 37 - DNA, 2661 bp, Bos taurus
  source 1..2661: mol_type = genomic DNA
tcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagtctgtgcaaggaaaagagacaaagccacggatcagaagcacatggccataactgaagattttgtgaactctcacaaggaaaaaatttgctctttgaacaataagaaggaactcactaaaatgtaactgagaactgttcaacaggttgaaagctgaaagatgccattggaactgacaaaatgtttcttaaacataaatgatgaaacagtgaaactacataatatctcctctggctgaaacattcaagaagtttaaaatgcttaagttaaaaataaaatcctagtaaacaatggacttactgtgcaacatagagaatatcttacgataacctgtaatggaaaagaatctgaaaaagaatgtatataactgaatcactttgctgtaaactagaatctgacacaacactgtaaatcactacacttttctgttgcatgccaaagattatttaataacgtcattaaaaaattattttaataatta

SEQ ID NO: 38 - protein, 679 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQSVQGKETKPRIRSTWP

SEQ ID NO: 39 - DNA, 7434 bp, Bos taurus
  source 1..7434: mol_type = genomic DNA
acaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatggagaatttgcagccttcaagtagccacaccatcatgacagcatctactcttatttcttaagtcttgtgttcgtacaatttgttaacatcaaaacacagttctgttcctcaactctttttaaagttaaaatttttcagtgcataagctggtgtggaacagaaggaaatttcccatccaacaaaagagggaagaatgttttaggaaccagaattctctgctgccagtgtttcttcttcaacacaaatatcacaagtctgcccactcccaggaagaaagaggagagaccctgagttctgaccttttgatggtcaggcatgatggaaagaaactgctgctacagcttgggagatttgctctgggaagtctgccagtcaactttgcccttctaaccaccagatcaatatgtggctgatcatctgatggggcagttgcaatcaccaagccttgttctctttcctgttctgggattgtgttgtggaacccttttccctagccaccaccagttcatttctgagggatggaacaaaaatgcagcatgcccttcctgtgtggtgcatgttcagtccttgacaaatttttaccaaaatgaagctactttatttaaaaggagggtgagaggtgaggaggtcactttgggtgtggcggaaagggaatgctgcatctttttcctgggctgctggggctctggccttggcttgccagccggaagcgctggcacgcatcgccttcttttcccattgggtccagcaatgaagacgagtgtttggggttttttttttctccaccatgtagcaagttctcaggaaaatacaattgatatcttcctcctaagctcttccaatcagtcaccaagtacttatgtggttactttgtccagggcacaaaatgcctgtatctaattaaaagcctacaaaactgcttgataacagttttgaatgtgagacatttatgtaatttaaatgtaaggtacaagttttaatttctgagtttcttctattatatttttattaaaaaaagaaaataattttcagattgaattggagtaaaataatattacttcccactagaattatatatcctggaaaattgtatttttgttacataagcagcttttaaagaaagatcattacccttttctctacataaatatatggggagtcttagcctaatgacaaatatttataatttttaaattaatggtacttgctggatccatactaacatctttactaatacctcattgtttcttccaacttactcctacactacatcctacatcttcttcctagtcttttatctagaatatgcaacctcaaataaaaatggtggtgtcctcattcattctcctccttccttttttcccaagcctgatcttcaaaaggttggttaatttggcagctgagttcctccccaggcagagaatagaccaattttaggtgtattgggactgagggaggatgtgtaaagattaacatcagtaaagaaccgctgtggagtaattaagaactttgttctttataactggagaatataacctaaccctaacatccctcagcctttactaaagtgtggcgtaaatcacagtagtagcaaagaaagtgactctggatgtgttcctggccagtacctcccttatcatgaatgtagactctctcatcaagatttaggaatataaatcaaatcaaatgtgcccagccaagctatgtagtaagggacttgaacaatattaggcagaacctataaaataaatcagggaattagaaattatttaaagttttcaaattgtaaattgccccggtgtctttcagcctactgccattatttttgctacaatacctacatttcagaggagggcctactgaaaattccatgcaagtggaaaataatcctcaagttattaatgagtttgaaaagcaatgagttcttaagtctttgtgagtagagcaagatcctacaaaattcagaaatagtaaaaatggattcatgctgatttgaagagcatctgtgtgcataatataatgctgcatctcttttaaaagcagtctatttttctttttaaatttgtccccatagatgcttttgaacatgaacatgcttatgttaccttttccgaggttgggaagagccaggagctctcaggcagggccccctccctcagctgggcaggagctgctcaggaggagctagttatagaggaagcttagcgttggcattttcaaaattcaaggtgataacgctttcttcttcctttctgttttagaatagattgctgtctgatttgaaaaagggaaatagatttgatctcaaatgaatctgtgcccagaagccaggctcagggtattcagagatttgtatagtgccctcaaaaaataacaaaattttagctttccttttttcttcttttctccatcaaattcttttttctctagtttacaaatgacatggaaaaggaatttcccctgagttttgtatgcctttttttttttggcttagactatagataggcgtgttgagctcctaagaaaatacaaggaggaactctttgttgtgcagagcactttatgagtagtttgtgtggataatatgtgactgcttccctgacgagcttgtgaggctgtacttatgtctttcctgtaaggcagcttcagtgccttctgtagtgtatataaggaaagattacgccttctgaaaaatctcagagcaaccataagattattttaaaatatgtagtatgactgatggactttttcatcattaaattagtctagcatctaaacttttaccactgaaataatattgaccaaaaagcaatttataaaaggtatttgtgaatagaaaatacaatgtgatcatttgtacttatgtgcaccttaaaagaggaattctgtctagctgtcaaattctggttccttaacatccagtccttgattgtgattgagatctggtaggacgtgctggggcacgctagcagataaaatcccgtatactttaggatagatgttacatttatgtcagtgttggcaaagagcattgtgtagtaataaagaattcaagacttcagcaatgtcaacctgaaactttgtaaatatttcctagattgttatttgatgcagtcacagctctttatcacacaatgttgtctttccctcatcaggcaattttagaactgctgcacacccctcctcagatctcacctgcccctcctgtacattcacctctccagccttgtgcacacctcatttagctttagtttgaaacacattgcagggttcaggtgacctcttcaaaaactacctcctcagaatgaggtaatgaatagttatttattttaaaatatgaaaagtcaggagctctagaatatgaagatgatctaagattttaacttttatgtatacttgttgagcactctccttttgtcctaaagggcattatacatttaagcagtaatactgaaaaatgtagctcagagtaactgaatgttgttgaaagtggtgccagaatctgttttaggggtacgtatcagaatcttaatcttaaatcggttacatgaaattaaatagttaatggtaacacttgactaacagatataattttaattttcggtaggcttttagcaagacagtaagtacatcttcataatgagttagccacagcttcatcacatgcacagattttcctgttgagagactgcccagttaagagggtagaatgatgaaccatttttcaggattctcttctttgtccaaactggcattgtgagtgctagaatatcagcactttcaaactagtgattccaactattaggctattaaaaagcaaaacaaaccaaacaaaccatagccagacatgggaagtttactatgagtataaacagcaaatagcttacaggtcatacattgaaatggtgtaggtaaggcgttagaaaaataccttgacaatttgccaaatgatcttactgtgccttcatgatgcaataaaaaaaaaaaaaatttagcataaatcagtgatttgtgaagagagcagccaccctggtctaactcagctgtgttaatattttttagcgtgcaatttagactgcaaagataaatgcactaaagagtttatagccaaaatcacatttaaaaaatgagagaaaacacaggtaaattttcagtgaacaaaattatttttttaaagtacataatccctagtatagtcagatatatttatcacatagagcaaataggttgaaatcacaattcagtgacatttctagagaaactttttctactcccataggttcttcaaagcatggaacttttatataacagaaatgtgtgacggtcattttaaattgctgtagtttggggctgaagtactgtgtgctgggcagcaatcacatgtattaactagtgagaaaggagaaattaagatataggacagaatttgattttcttgttcccagattactgctgccaacctagacactgagtttccagaggctgaaacgtaaacttgcagctcagcaactgttttgcaaagttagtgggactgtcctgcttatgctgttcaaaaatgctctgagggccaggtggggcctccaggggctcctctctgaggggacatcagactagctaacgacctggcgggcggatgtgaaccggacacactccatggtgtgcttcttgtatcggtccctcgccaccctcaagaaaggcttcagcgggttctctagacgtctccactaaggtgtgttactaacagccatgggttgttgagcacccgaggagtgcaatagcatctctgcatgattgtatattggcccgaagagaatgaagtggccagtgtactcatgttccatgttgctagctctggtaaactgaaaatactggtaagatttttgttttatcagtacactagagagtaagctttgttttgttgtttttagataatgttttcacttccatttggaaagacatttaaattgagtttcagtcctaaattttgccagtcatggtaattagcagtttctatcaggtatttttaaggtagaagaggatagaaacataagttctaaaagcttaaggtaaccgtggtttattttaaaatgtttaggggtggttagtctctacctcaaaaaaagtgagtgaatcttttatttcagcattcacaagttcggctgttgtttttgtaatacattttttttttaaccttttgacccccctttacctaagtgtcaatgtagttttattaattactaagtcagtttcattaaaatgtttatttagcagttttgactaattgcaatgattaatatagccagttgtgcatgaggacacagccagtgagtatatctgggttttttttgtgatgctttttttcttaagacttctgtagatttatgaagtactcattgaaaacaactaaaatacgtttattcgtgttaatatggaaaaaaaaaaa

SEQ ID NO: 40 - protein, 603 aa, Bos taurus
MMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYGEFAAFK

SEQ ID NO: 41 - DNA, 2295 bp, Equus caballus
  variation 79: note = n is a, c, g, or t
  source 1..2295: mol_type = genomic DNA
atgaagacgctgagcggcggcggcggcggcgcggagcagggccaggctctgttcaacggggacatggaacccggaggcnccgcgccggcgcccgcggcctcgtcggccgcggaccctgccattcccgaggaggtatggaatatcaaacaaatgattaaattgacacaggaacatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagttctgcatcaacggacaccgttacatcttcttcctcttctagcctttcagtgctaccttcatctctttcagtttttcaaaatcccacagatgtgtcacggagcaaccctaagtcaccacaaaaacctatcgttagagtcttcctgcccaacaaacagaggacagtggtacctgcaaggtgtggcgttacagtccgggacagtctaaagaaagcactgatgatgagaggtctaatcccagagtgctgtgctgtttacagaattcaggatggagagaagaaaccaattggctgggacactgatatttcctggctcactggagaggaattgcatgtagaagtgttggagaatgttccacttacaacacacaactttgtacggaaaacttttttcaccttagcattttgtgacttttgtcgaaagctgcttttccagggtttccgctgtcaaacatgtggttataaatttcaccagcgttgtagtacagaggttccactgatgtgtgttaattatgaccaacttgatttgctgtttgtctccaagttctttgaacaccacccagtatcacaggaggaggcctccttagcagagactgcccttacatctggatcatccccttctgcacccccctccgattccattgggccccaaattctcaccagtccatctccttcaaaatccattccaattccacagcctttccgaccagcagatgaagatcatcgaaatcagtttggacaacgagaccggtcctcatcagctccaaatgtacatataaacacaatagaacctgtcaatattgatgacttgattagagaccaagggtttcgtagtgatggaggatcaaccacaggtttatctgccaccccccctgcctcattacctggctcactcactaatgtgaaggcattacagaaatctccaggacctcaacgggaaaggaaatcatcttcatcctcagaagacaggaatcgaatgaaaactcttggtagacgggattcaagtgacgattgggagattcctgatgggcagatcacagtgggacaaagaattggatctgggtcatttgggacagtctacaagggaaagtggcatggtgatgtggcagtgaaaatgttgaatgtgacagcacccacacctcagcagttacaggccttcaaaaatgaagtaggagtactcaggaaaactcgacatgtgaatatcctactcttcatgggctattcaacaaagccacaactggctattgttacccagtggtgtgagggctccagcttatatcaccatctccacatcattgagaccaaatttgagatgatcaaacttatagatattgctcggcaaactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccgtatagctttcaatcagatgtatatgcctttgggattgttctgtatgaattgatgactggacagttaccttattcaaacatcaacaacagggaccagataatttttatggtgggaagaggatatctatctccagatctcagtaaggtacggagtaactgtccaaaagccatgaagagattaatggcagagtgcctaaaaaagaaaagagacgagagaccactcttcccccaaattctcgcctctattgagctgctggcccgctcattgccaaaaattcaccgcagtgcatcagagccctccttgaatcgggctggcttccagacagaggattttagtctatatgcttgtgcttctccgaaaacacccatccaggcagggggatatggtgcgtttcctgtccactga

SEQ ID NO: 42 - protein, 764 aa, Equus caballus
  VARIANT 27: note = X can be any naturally occurring amino acid
MKTLSGGGGGAEQGQALFNGDMEPGGXAPAPAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPVSQEEASLAETALTSGSSPSAPPSDSIGPQILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 43 - DNA, 2678 bp, Gallus gallus
  source 1..2678: mol_type = genomic DNA
tccccctccctcgccccagcgcttcgatccaagatggcggcgctgagcagcggcagcagcgccgagggggcctcgctcttcaacggggacatggagcccgagccgccgccgcccgtgctgggcgcctgctacgccgggagcggcggcggcgacccggccatcccggaggaggtgtggaatatcaaacagatgattaaattaacacaagaacatatagaagcgctgttagacaagtttggaggagagcataacccaccatcaatatatttagaggcctatgaggagtacaccagcaaactagatgctctacagcagagagaacagcagttattggaatccatgggaaatggaactgatttctctgtttccagttcagcttcaacggacacagttgcatcatcttcctcctctagcctctctgtagcaccttcatccctttcagtttatcaaaatcctactgatatgtcgcggaataaccctaagtctccacagaagcctattgttagagtcttcctgcccaacaagcaaaggactgtggttccggcaagatgtggggtgacagtccgagacagcctgaagaaagctctgatgatgagaggtcttattccagaatgctgtgctgtttacagaatacaggatggagagaagaagccaattggctgggacactgacatttcctggctaaccggagaggagttacacgtggaggtcttggagaatgtgccactcacaacacacaattttgtacgaaaaacattcttcacgttagcgttctgcgacttctgtcgaaagctgcttttccagggattccgatgccagacatgtggctacaaatttcaccagcgctgtagcacagaagtgccactgatgtgtgttaactacgaccaactcgatttgctgtttgtctccaagttctttgaacatcaccccatatcgcaggaggagaccaccttaggagagaccaccccggcatcgggatcgtacccctcagtgcccccatcagattctgttggaccaccaattctccctagtccttctccttcaaaatccattccaatcccacagcccttccgaccagcagatgaagaccatcggaatcagtttgggcaacgcgaccgatcctcttcagctcccaatgttcacatcaatacaattgagccagtcaatattgatgacttgattagagaccagggtgtacgaggagagggagcccctttgaaccagctgatgcgctgtcttcggaaataccaatcccggactcccagtcccctccttcattctgtccccagtgaaatagtgtttgattttgagcctggcccagtgttcagaggttcaactgcaggtttgtctgcaacacctcctgcatctttgcctgggtcacttaccaatgtgaaagcattacagaaatcaccaggcccccaacgggaaaggaaatcatcctcatcctcagaagacagaaataggatgaaaacccttggtcgacgagattcaagtgatgattgggaaataccagatgggcagatcacagttggacaaaggataggatctggatcatttggaacagtctacaaaggaaagtggcatggtgacgtggcagtgaaaatgttgaatgttacagcacccacacctcaacagttacaggctttcaaaaatgaagtaggagtgctcaggaaaacacggcatgtgaatatcctactttttatgggttattcaacaaaacctcagttggctattgttacacagtggtgtgaggggtccagcttatatcaccatctgcacataattgagaccaagtttgaaatgatcaaactaattgatattgcacgacagactgcacaaggcatggattatttgcatgccaagtcaatcatccacagagacctcaagagtaataatatttttcttcatgaagacctcacagtaaaaataggtgacttcggtctggctacagtgaaatcacgatggagtggatctcatcaatttgaacagttatctggatcaattctatggatggcaccggaagtgatcaggatgcaagacaaaaacccatatagctttcagtcagatgtgtatgcattcgggattgtgctttatgaactgatgactggacagttaccatactcaaacatcaacaacagggaccagataatttttatggtgggacgaggatatctatctccagacctcagtaaagtaagaagtaactgtccaaaagctatgaagagactaatggcagaatgcttgaaaaagaaaagagatgagagacctctttttccacagattcttgcctccattgagcttctggcccggtcgttgccaaaaattcaccgcagtgcatctgagccgtcactaaaccgggctggcttccagaccgaggatttcagtctgtatgcttgtgcttctccaaaaacgcccatccaagcagggggatacggtgggtttccagtacactgaaaagaaatgtgaaagcgtgtgcctgtttgctcatgtgctggtgtgttcctgtgtgtgcaacgcatacgtacgttctcagttcctaccagcgactttttaaggtttactgagggaatgaagactcatttcctaacatggggcattgaacgtcctgagcacaagtcagtgctggtaaggaatgtcttgggaacagctggcaagaagaattagaaggtacttaaagg

SEQ ID NO: 44 - protein, 806 aa, Gallus gallus
MAALSSGSSAEGASLFNGDMEPEPPPPVLGACYAGSGGGDPAIPEEVWNIKQMIKLTQEHIEALLDKFGGEHNPPSIYLEAYEEYTSKLDALQQREQQLLESMGNGTDFSVSSSASTDTVASSSSSSLSVAPSSLSVYQNPTDMSRNNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPISQEETTLGETTPASGSYPSVPPSDSVGPPILPSPSPSKSIPIPQPFRPADEDHRNQFGQRDRSSSAPNVHINTIEPVNIDDLIRDQGVRGEGAPLNQLMRCLRKYQSRTPSPLLHSVPSEIVFDFEPGPVFRGSTAGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGGFPVH

SEQ ID NO: 45 - DNA, 4454 bp, Homo sapiens
  source 1..4454: mol_type = genomic DNA
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattctt

SEQ ID NO: 46 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 47 - DNA, 1326 bp, Rattus norvegicus
  source 1..1326: mol_type = genomic DNA
gccgttcatggcggtttcggggtctccaacagcttctcaggttgaaatccaaaagcctcccgaggcggggtctgcggagtttgagatttttgcaggtgtgaaatgactgagtacaaactggtggtggttggagcaggtggcgttgggaaaagtgctttgacaatccagctaatccagaaccactttgtggatgaatatgatcccaccatagaggattcttaccgaaaacaagtggtgattgacggtgagacctgtctactggacatactggacacagctggacaagaggagtacagtgccatgagagaccaatacatgaggacaggcgaagggttcctctgtgtgtttgccatcaataatagcaaatcctttgcagatattaacctctacagggagcaaattaagcgcgtgaaagactctgatgatgtacccatggtgctggtagggaacaagtgtgacttgccaacaaggacagttgacacaaagcaagcccacgagctggccaagagttatggaattccattcattgaaacctcagccaagacccgacagggtgtggaggatgccttttacacgcttgtaagggagatacgccagtaccggatgaagaagctcaacagcagtgaggatggcactcaaggctgtatggggctgccctgtgtggtgatgtagtaagaccctttaaaagttctgtcatcagaaacgagccactttcaagcctcactgatgccctggttctgacatccctggaggagacgtgtttctgctgctctctgcatctcagagaagctcctgcttcctgcttccccaacttagttactgagcacagccatctaacctgagacctcttcagaataactacctcctcactcggctgtccgaccagagaaatgaacctgtttctccccagtagttctctgccctgggtttcccctagaaacaaacacacctgccagctggctttgtcctccgaaaagcagtttacattgatgcagagaaccaaactatagacaagcaattctgttgtcaacagtttcttaagctctaaggtaacaattgctggtgatttccccctttgcccccaactgttgaacttggccttgttagttttgggggaaatgtcaaaaattaatctcttcccgagaatagaattagtgttgctgattgcctgatttgcaatgtgatcagctatattctataagctggcgtctgctctgtattcataaatgcaaacatgagtactgacgtaagtgcatccctagtcttctcagctgcatgcaattaaatccaacgttcacaacaaaaaaaaaaaaaaaaaaaaaaaaa

SEQ ID NO: 48 - protein, 189 aa, Rattus norvegicus
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSEDGTQGCMGLPCVVM

SEQ ID NO: 49 - DNA, 4470 bp, Mus musculus
  source 1..4470: mol_type = genomic DNA
gggactggggcgccttgggcgcctagtgattacgtagcgggtggggccggaagtgccgctccctggcgggggctgttcatggcggtttcggggtctccaacagcttctcaggttgaagtccaaaagcctcccgaggcggggtctgcggagtttgaggtttttgctggtgtgaaatgactgagtacaaactggtggtggttggagcaggtggtgttgggaaaagcgccttgacgatccagctaatccagaaccactttgtggatgaatatgatcccaccatagaggattcttaccgaaagcaagtggtgattgatggtgagacctgcctgctggacatactggacacagctggacaagaggagtacagtgccatgagagaccagtacatgaggacaggcgaagggttcctctgtgtatttgccatcaataatagcaaatcatttgcagatattaacctctacagggagcaaattaagcgtgtgaaagattctgatgatgtccccatggtgctggtaggcaacaagtgtgacttgccaacaaggacagttgacacaaagcaagcccacgaactggccaagagttacggaattccattcattgagacctcagccaagacccgacagggtgtggaggatgccttttacacactggtaagggagatacgccagtaccgaatgaaaaagctcaacagcagtgacgatggcactcaaggttgtatggggctgccctgtgtgctgatgtagtaagacactttgaaagttctgtcatcagaaaagagccactttgaagctgcactgatgccctggttctgacatccctggaggagacctgttcctgctgctctctgcatctcagagaagctcctgcttcctgcttccccgactcagttactgagcacagccatctaacctgagacctcttcagaataactacctcctcactcggctgtctgaccagagaaatagacctgtctctcccggtcgttctctgccctgggttcccctagaaacagacacagcctccagctggctttgtcctctgaaaagcagtttacattgatgcagagaaccaaactagacatgccattctgttgacaacagtttcttatactctaaggtaacaactgctggtgattttcccctgcccccaactgttgaacttggccttgttggtttggggggaaaatgtcataaattactttcttcccaaaatataattagtgttgctgattgatttgtaatgtgatcagctatattccataaactggcatctgctctgtattcataaatgcaaacacgaatactctcaactgcatgcaattaaatccaacattcacaacaaagtgcctttttcctaaaagtgctctgtaggctccattacagtttgtaattggaatagatgtgtcaagaaccattgtataggaaagtgactctgagccatctacctttgagggaaaggtgtatgtacctgatggcagatgctttgtgtatgcacatgaagatagtttccctgtctgggattctcccaggagaaagatggaactgaaacaattacaagtaatttcatttaattctagctaatcttttttttttttttttttttttttggtagactatcacctataaatatttggaatatcttctagcttactgataatctaataattaatgagcttccattataatgaattggttcataccaggaagccctccatttatagtatagatactgtaaaaattggcatgttgttactttatagctgtgattaatgattcctcagaccttgctgagatatagttattagcagacaggttatatctttgctgcatagtttcttcatggaatatatatctatctgtatgtggagagaacgtggccctcagttcccttctcagcatccctcatctctcagcctagagaagttcgagcatcctagaggggcttgaacagttatctcggttaaaccatggtgctaatggaccgggtcatggtttcaaaacttgaacaagccagttagcatcacagagaaacagtccatccatatttgctccctgcctattattcctgcttacagacttttgcctgatgcctgctgttagtgctacaaggataaagcttgtgtggttctcaccaggactggaagtacctggtgagctctggggtaagcctagatatctttacattttcagacccttattcttagccacgtggaaactgaagccagagtccatacctccatctccttccccccccaaaaaaattagattaatgttctttatatagcttttttaaagtatttaaaacatgtctataagttaggctgccaactaacaaaagctgatgtgtttgttcaaataaagaggtatccttcgctactcgagagaagaatgtaaaatgccattgattgttgtcacttggaggcttgatgtttgccctgataattcattagtgggttttgtttgtcacatgatacctaagatgtaactcagctcagtaattctaatgaaaacataaattggataccttaattgaaaaaagcaaacctaattccaaaatggccattttctcttctgatcttgtaatacctaaaattctgaggtccttgggattcttttgtttataacaggatcttgctgtgtagtcctagctggcctcaaactcacaatactcttcctggatcaatctcccaagtgctgggattacaggcacattccaccacacacacctgactgagctcgttcctaatgagttttcattaagcaaattccccatcaccttgaaactaatcagaagggggaagaaacatttgctatgctcctgagtgctaacactgggatcattcacatggggtttgcattcctaggcaaactaaactgctgccttttacaacaaggctcagtcatcttcctgaagctgctgagaccagcacttggtcttgttttgttttaatatgtctatatgactggtggtggatccctaaatagtttattaattaaactccagttaaggagaaagttactcaccttgacccgtttgaccatatcccgtgtgtgtgtgtgtgtgtgtgtgtgtgtgtgtgtgtgtgtgtgtgtgcacgcgtatgtacgtacgtatgtatgtaggtatgtaggtggtttccagtataaacacagaaacaaatggagccaattcaggtttcagatgcccttactaacatatattcccacggggtgtgggttttggcacaacagtgacaaacttaaaagccaagtaagagccgggcgtggtggcgcacgcctttaatcccagcacttgggaggcagaggcaggcggatttctgagttctaggccatcctggtctacagtgagttccaggacagccagtgctacacagagaaaccctgtctcgaaaagccaaaaaaaaaaaaaaaaaaaaaaaaaaaaagccaagtaggtccagttggtatagtatcaaagtgtttttagagtaattagtgaaggtctgctttacctcaaagttgcagagcctctcttcctgagtttaagtgcctggccggcagtcacaaattaacatgttgctgtaaggcagttagttgaagctttgttcacacattggagagtatgaaaataaagtgttctaagagcgctgatactggatctgtgtaaacctggtaaatgccgtttgtccaggacttagcgtgtgtgagttggtagctcagtacgagtttactagttccgcagtgtgtacaatggaggcgggtttgttttagctggccacctgtagaatcagcctttaaactgctgtgaactttgtcatgacttgaatatgaagatagacaaaaactctgtaaagacaaatgtttgttttcccccttacagaacgtgtgagcttggttttatcttcctttgtatttagtcataacctctcaagctggcagctccgaccaaggatcagaagctgtgtgcgttccacctggtggaattagctcagctctatatgagaagtggagttaatggaaaacgtgttgactgggtggtttctatttaaaagagtgatgataattcttgaacagtagtttttattttgctatttctttaagctgactgatgtgccacaaaattattttaaggtatttgtgttttaagagtgttctcatgagattagttgtagatattttttaaaatacaactggtttttaaaatctgagtattgctctaagcaagtgtttagactcttacgggaaggtgggtggaagttgtttggcttccgtatttccatgcgtgccgtcagacataggtcagaacgccaactgtgcatcctgctgtttaaagacctcttggcctctgtgaccctcatgaaggggctgatattttaagttgactgtttgattgtaaattaatcctttctaatttttaaagacttgcttgactgttttccttgttaaataattttaaaaaaataaaaaactggaagttctttgcttaactgta

SEQ ID NO: 50 - protein, 189 aa, Mus musculus
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVLM

SEQ ID NO: 51 - DNA, 570 bp, Cavia porcellus
  source 1..570: mol_type = genomic DNA
atgactgagtataaactggtggtggttggagcaggtggtgtcgggaaaagtgcactgaccatccagctaattcagaaccactttgtcgatgaatatgatcccaccatagaggattcttaccgaaaacaggtggttatagatggtgaaacttgtctgttggatattctggatacagctggacaagaggagtacagtgccatgagagaccaatacatgaggacaggcgaaggcttcctctgtgtgtttgccatcaataatagcaaatcatttgcagatattaacctctacagggagcagattaaacgagtaaaagactcagatgatgtacctatggtgctggtagggaacaagtgtgatttgccaacaaggactgttgacacaaaacaagcccatgaactggccaagagttacgggattccattcattgaaacctcagccaagaccagacagggtgttgaagatgcattttacacactcgtaagagaaatacgccagtacagaatgaaaaaactcaacagcaatgatgatgggactcaaggttgtatggggttgccatgtgtggtgatgtaa

SEQ ID NO: 52 - protein, 189 aa, Cavia porcellus
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSNDDGTQGCMGLPCVVM

SEQ ID NO: 53 - DNA, 1220 bp, Cavia porcellus
  source 1..1220: mol_type = genomic DNA
gttccggggtcctcaacgtttctcagggttgagattctatatccttttgaagctggggcggcagagcttgaggttcttgctggtgtgaaatgactgagtataaactggtggtggttggagcaggtggtgtcgggaaaagtgcactgaccatccagctaattcagaaccactttgtcgatgaatatgatcccaccatagaggattcttaccgaaaacaggtggttatagatggtgaaacttgtctgttggatattctggatacagctggacaagaggagtacagtgccatgagagaccaatacatgaggacaggcgaaggcttcctctgtgtgtttgccatcaataatagcaaatcatttgcagatattaacctctacagggagcagattaaacgagtaaaagactcagatgatgtacctatggtgctggtagggaacaagtgtgatttgccaacaaggactgttgacacaaaacaagcccatgaactggccaagagttacgggattccattcattgaaacctcagccaagaccagacagggtgttgaagatgcattttacacactcgtaagagaaatacgccagtacagaatgaaaaaactcaacagcaatgatgatgggactcaaggttgtatggggttgccatgtgtggtgatgtaacaagatatttaacaaagttctatcagaaaagagccactttcaagctgcactgataccctggtcctgacttccctggaggagaagtatccctgttgctctcttcatctcagagaagctcctgctgtttgtccacctctcagtgtatgagcacagtctctgcttgagaacttctcagaataactacctcctcacttggttgtctgaccagagaaatgcacctcttgttaattccccaataattttctgccctgggctctccccaacaaaaaacaaacacttctgccatccaaaaagcaacttggtctgaaacagaaccaaactgtagattgaaattctcttaaaaagtcttgagctctaaagttagcaaccgctggtgatttttattttcctttttatttttgaacttggaactgacctatgttagattttggagaaatgtcataaagtactgttgtgccaagaagataattatgttgctgaatggttgatttatagtgttatcagctatattttacaaactggcatctgctctgtattcataaatacaaaaatgaagccagg

SEQ ID NO: 54 - protein, 189 aa, Cavia porcellus; identical to SEQ ID NO: 52

SEQ ID NO: 55 - DNA, 1307 bp, Canis lupus
  source 1..1307: mol_type = genomic DNA
tgattacgtagcgggcggggccggaagtgccgctccctagtgggggctgttcatggcggttccggggtctccaacctttctcctagttgtggtcctaaatacgtcggaagcggaggcggcgaagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttaccgaaaacaggtggttatagacggtgaaacctgtctgttggatatactggatacagctggtcaagaagagtacagtgccatgagagaccaatacatgaggacaggcgaaggcttcctctgtgtatttgccatcaataatagcaaatcatttgcagacattaacctctacagggaacagattaagcgagtaaaagattcagatgatgtacctatggtgctagtaggaaacaagtgtgatttgccaacaaggacagttgacacaaaacaagcccatgaactggccaagagttatgggattccattcattgaaacctcagccaagaccagacagggtgtcgaggatgccttttacacactggtaagagaaatacgtcagtaccgaatgaagaaactcaacagcagtgatgatgggactcaaggttgtatggggttaccatgtgtggtgatgtaacaagacacttttaaagttctagcatcagaaaagagccactgtcaagctgcactgacaccctggtcctgacttccctggaggagaagtattcctgttgctatcttcagtctcacaaagaagctcctgctacttccccaactctcagtagatcagtacaatgttctctatttgagaagttctccgaacaactacctcctcacttggttgtctgaccagagaaatgaacctcttgttccttcccgctgtttttccaccctgaattctcccccaacacacataaacaaacctctgccatcccaggtttttcatctgaaaaataattcatgctctgaaacagagaacaaaactgtagacatgaaattctgtaggaaacaaggtcttgagctcaaaagtagcaactgctggtgacctttttttcccccctttttactgttgaacttggaactatgttggtttttggagaaatgtcataagttactgttttgctgagaatatagttaagttgacatttggtttgtttgtaatatcattagctattttctataaattggcatctgctctgcattcataaatacacgagtgaattctga

SEQ ID NO: 56 - protein, 189 aa, Canis lupus; identical to SEQ ID NO: 46

SEQ ID NO: 57 - DNA, 3104 bp, Felis catus
  source 1..3104: mol_type = genomic DNA
aaaaaataaataaatttaagaaaccattttaaaattatgcacagttgcagcctggaaaacttaaggtggcgccttatagtatcaatcttaggagctttatttggtgcatttaacgcaactggtaattgcaaaatccacttcgcctgtgtaagtgaaaaatatagactgttatcttgttggccctatgaaattctgcacttggtatttagcatatactctaccttcattactatctggcaagatgttctgccttagcactcagttgcattcttttccttttctttcctgttcattatgctttaattctgaggaccatatgagggtagaatatattaaaaattacaaaaattataaaaatttgtataggcaaaccatttccttaagttgatggccaaatgttaaaatgttatttttcatatcatttataatcttgtcacagtccacttaacgaagtttggttagatttcagtgaaaattatcttccagagtagtttttttttttttttcctgggattagggaggggggtaactttactgcaattagtatgtatggtgcagaatttcatgcaaatgaggtgtgccagcagtgtggtaatttaatcgtatttaaacaaaaacaaacaaaaaaaaaacgaatgcacaaacttgctgctgcttagatcactgcagcttctaggacccagtttcttttactgatttcaaaacaaaacaaaacaaaaaaataaaaaaagttgtgcctgaaatgaatcttgttttttttataagtagccgcctggttcctgtgtcctgtgaaatacaggcacttgacccttggtgtagcttctgttcgactttatatcacgggaatggattggtctgatttcttggccctcatcttgaattggccacatccagggtccctggccagtggactgaaggctttgtctaagaggacaagggcagctcaggggatgtgggggagggcgcttttatcttccccgttgtcgtttgaggttttgatcttctctgggtaaagaggccgtttatctttgtaaacacaaaacatttttgctttctccagttttctgttaatggcgaaagaatggaagcgaataaagttttactgatttttgagactctagcacctagcgctttcatttttgaaacgtcctgtgtgggaggggcgggtctgggtgcggcccgccgcgtgactcctgagtcgggggcccacgtggctggggcggggactcggacgccccgggcgccgactgattacgtagcgggcggggccggaagtgccgctccctagtgggggctgttcatggcggttccggggtctccatcctttttcccagttgttctaaatcagtcggaagcggaggcagcgaagtttgaggttctcgctggtgtgaaatgactgagtacaaactggtggtggttggagcaggtggtgttgggaaaagcgcactgacaatccagctaatccagaaccactttgtagatgaatatgatcccaccatagaggattcttaccgaaaacaggtggttatagacggtgaaacctgtctgttggacatactggatacagctggtcaagaagagtacagtgccatgagagaccaatacatgaggacaggcgaaggcttcctctgtgtatttgccatcaacaatagcaaatcatttgcagatattaacctttacagggaacagattaagcgagtaaaagactccgatgatgtacctatggtgctagtaggaaacaagtgtgatttgccaacaaggaccgtcgacacaaaacaagcccacgaactggccaagagttatgggattccattcattgaaacctcagccaagaccagacagggtgttgaagatgccttttacacactggtaagagaaatacgtcagtaccgaatgaagaaactcaacagcagtgatgacgggactcaaggttgtatggggttaccgtgtgtggtgatgtaacaagatacttttaaagttctagcatcagaaaagagccactgtcaagctgcactgacaccctggtcctgacttccctggaggagaagcgttcctgttgctattttcagtttcacaaagaagctcctgctatttccccaactctccgtagatcagtacattattctctgtttgagaagttctccgaataactacctcctcacttggttgtctgaccagagaaatgaacctcttgttactccccactgtttttccaccctggttctcccccagcacatataaacaaacctcccaggtttttcatctgaaaagtaattcatgctctgaaacagagaaccaaactgtagacatgaaattctgtaggaaacaatgtcttgagctctaaagtagcaactgctggtgactttttttttttttttttcctttttactgttgaacttggaactatgttggtttttggagaaatgtcgtaagttactgttttgctgagtatatagttaagtttaccattcggtttgtttgtaatgtcattggctatactctgtacctggcatctgctctgcattcataaatacaaaagtgaattctgacttttgagtctatcctagtgttctcaacttccacataattaaatctaacttttgcagcaaagtgcctttttcctagaagtggtttgtagatttgctttataatactttggtggaatagatgtctcaaaaaccattatacatgaaaatgaatgtctgagatacgtctatgatctgtctacctttgagggaaaaatataccgacataatagcagatgccatgtcttacgtgtatgaagttggatttccagagacctgatttgggtctcttccaagagaaagatgaaactggaaacaattatgaataacttcacttaatttttacctaatctctacttcggggtgggagggcagggagtaggttaccacttacaaaatatatgcaatttgtttcttctagcttactgataatgaacttccattcttatttaaatttaggtcatatcctaaagctttacatttgcaggtgttcgaaattgtaagtttaatgcagttttatttaatagctatgatcaatgattttcaagcctcagatgtattaacggacacattttcact

SEQ ID NO: 58 - protein, 189 aa, Felis catus; identical to SEQ ID NO: 46

SEQ ID NO: 59 - DNA, 4283 bp, Bos taurus
  source 1..4283: mol_type = genomic DNA
ggccgctccctagtgggggctgttcatggcggttccggggtctcccaacaattttcccggttgtggtcgtaatctatccgaagtggaggcagtggagctagaggttcttgctggtgtgaaatgactgagtacaaactggtggtggttggagcaggtggtgttgggaaaagtgcactgacaatccagctaatccagaaccactttgtagatgaatatgatcccaccatagaggattcctaccgaaaacaggtggttatagatggtgaaacctgtctgttggacatactggatacagctggacaagaggagtacagtgccatgagagaccaatacatgaggacaggcgaaggcttcctttgtgtgtttgccatcaataatagcaaatcatttgcagatattaacctctacagggaacagataaagcgtgtaaaggactcggatgatgtacctatggtgctagtaggaaacaagtgtgatttgccaacaaggacagttgacacaaaacaagcccatgaactggccaaaagttatgggattccattcattgaaacctcagccaagaccagacagggtgttgaagatgccttttacacactggtaagagaaatacgtcagtaccgaatgaaaaagctcaacagcagtgatgatggcactcaaggctgtatggggttgccgtgtgtggtgatgtaacaagatacttttaaagttctcacatcagaaaagagccactgtcaagctgcactgacaccctggtcctgacttccctggaggagaagtattcctgttgctatcttcagtttcaaaaagaagctcctgctatttccccaactctcagtagatcaatataatattctctatttgagaagttctcaagaataactacctcctcacttggttgtctgaccagagaattgaacctcttgttactcccagtatttttccaccctgggttctcccccagcacacacaaacgcacctctgccacccaggtttttcatctgaaaagcaattaatactctgaaacagagaaccaaactgtagaaacatgaaattctgtagaaaacaatgtcttgagctctaaagtagcaactgctggtgattttttttttttttttttcctttttattgttgaacttggaactatgttggtttgtggagaaatgtcataaattactgttttgctgagaatatagttaatgttgctctctggtttgtttgtaatgttatcagctatattctataaactggcatctactctgtatttagaaatacaaaaatgaatactgaccttttgagtctaccctcatcttctcgactttcttgtaattaaatgtaactttcacgatgaagtgccttttgcctgggagtgactcgtagacttcctttaaaatacttcagtggaatagatgtctcagaaactgttatacataagaataaatgtctgagatatgtctatgacccatctagctttgagggaaagatataccaatatgatagcagatgccatttcttacatctataacgttgattttctggagacctattttggggctctccgagagaaagatgagactataaatgattaggaataatttcacttaatttttacataacctccactttttgttttgtagtttactacctgcaaaacatataatttgattccttttagcttacagataatctaatgttaaatgaacttccattcatattttaatttggatcatatcaggaagtctacatttgcaggtgttcaaaaattgtaaaagtgtgatgcagttttatttaatagctgtgatcaatgattttcaagcctcaaatatgttaatagacacattttcactgtatatcatggtattaataattattgatgtatataattgtccttggtccccttctctgttcatcacctcatggcaatggcttgattaattatttcagctgagtaaagcatggtgctaatagaccagggtcacagtgtcaaaacttcagtgagccagtaagcatcacagagaaagaaattctttcacatttgctcaccattaactccagctaatagttttgccagatgtgtgtggttagtcctgcaaggaaaggagaagtcagttaatacaaattcttaaccaggactggaaaaacttgttttcctgagaagggtcagcttagaagtctttatctggactctatttttagccacatggaaatcaaattaagctgatcttttttctcaagtttttgagagtgaggatgcctcagatcaacatttttaaaatattctttattcttacgttcttttaagggtttaaaacaacgttgagtaattagtctgggcataccaggtaacaagctgataagtttgtgctgaacaagaagtagcctttggattgaaattgctgttttgagaagggatagaaaatataattaataattatgagacttgacttttctatttgcagataatatcctgataattctgatgaaaatagacttggataatttttgataaaagaatcgttccaaaatggccacttgctgttcttgtcttctaatgtgtaaatacttactgaggtcctcttctaatatgagttgtcatttattaagcaaattccacattgccttgaaatgaattcggaagagaagaaaaagtcatagtatacccagagaatgaaaaatccagagaattgtgctccttagtgttaattctgaagccttcgtagtccacacccatagacagaaactctctgccactttgcttctgctcctcttggagcattgcgctgtcatttccttgaggatagattgaggcttgtcaactcagttgtattgtcttcctcctcttcctcttgtctgtgtgactgacagtgtgactcttactaatgtcagatgcggggatgcggggaggtgggggggagtagctcattttaggctcttgcaccctttaccgttgtatgtgtgtgtcttttagttttctcaagaatgttctaagcacagaagtatctaaatggggccaaaattcagacttgaaaatgttcttttaatagcttcttaaaaagttacactttggtgtgaattttggcaggatagagtgacaaactcttaaacgctgaataacttcagttagtgtgttatagtttttagaatatgtttgtgattgctgaaaacaattatagtttacctcaaaatctgaaagtctctttccccaagttaagtgcctggccagctgtcaaagattacatattactttatgtttgtttgttttttaaaggttgcacattcaagattgtgaaaataaggtgttctgtctgaaagctaccatgcctgtctgtaaatgaatccactgagtgctgtacttgttccaacagcttactacagaatgctacttggtaatatcatactcgttacagttttcacttcaggagtgtactaggtagaatgatcctgtgtgtattgtagtgggctccatgtttagtcttttcagcatcctttaaactgctgtgaatttttgtcttgacttgaaagcaaggatagagaaacactttaaagagatactttgggtttttttccattccagaattggtgagcatagttagattttgctttacatttacagtcatgaactcttaagctggcagctacaaccaagaaccaaaagagggtgcattctgcttcttgtaattcatctttgctaataaattatgagaagcaaagataattaattagagaaactattttatttgggtggtttctataaacaagggactataattcttaaacattatttttcatttttgctgtttctttaagaaacctaatgtgccacaacattattttaaggtgtttcttaaaagaattgtttttaaaagtgttctcattttcagagtaattgtagatatatttcaaaatataactgataatttttaaaggcctgagtactgacctaagaagcagttgtatgaattctctggggggaagggaggagctcagtgaaagttgtatgacttttatatttctgtgccatcaaataaaggtaaaaatgtcttttgtgcagttttgctgttcaaacagaaactattggcctccttggccctaaatgaaagggctggtattttaagttgactattttattgtaaattaatccatcttaatttttttaaatttggttgaatgttctcttgttaaatgtttaaaaaataaaaactggaagttctttgcttagtcataaaaaaaaaaaaaaaaaaaaaaaaaaaaaaaaaaaaaaaaaaaaaaataaaaaaaaaaaaaaa

SEQ ID NO: 60 - protein, 189 aa, Bos taurus; identical to SEQ ID NO: 46

SEQ ID NO: 61 - DNA, 4825 bp, Gallus gallus
  source 1..4825: mol_type = genomic DNA
gcgccgggaccggaagccggaagctttgcagaagggtgttccgcgttcgcggtgcgggagcggtcagccggggtggcggggctggggccggccggggcaggcggctccgcgctccgcactgggccgctgggagggcgatgactgaatacaagctggtggtggtgggagctggcggcgtcgggaagagcgcgttgaccatccagctcatccagaaccacttcgtggacgagtacgaccccaccatcgaggattcgtacagaaagcaggttgtcatcgatggagagacgtgcttgttggacattctggacactgcaggacaggaagaatacagtgctatgcgtgatcagtacatgagaactggggaaggattcctttgtgtgtttgccattaacaacagtaaatcattcgctgatattaacctttacagagagcaaatcaagagagtgaaagattcagacgatgtgccaatggtgctggtggggaataagtgcgatttgccaacaaggacagtagacaccaaacaggctcaagagttagcaaaaagctacggcattcccttcatagagacatcagccaaaacgagacagggtgtggaagatgcgttttacacactggtgagggagattcggcagtaccggatgaaaaagctcaacagcaacgaagatgggaatcagggctgtatggggttgtcctgcattgtgatgtgataagatgccaggttcagatgtagctgctggacaagtctcgatgctactgtattgtgtctcatgctgatgccctgcagtattttggtgccagcgaccagactcttggtaccagttaattagctcaggatcctttcctgtgctccatctgaagaaaacatctctggtatctacctccttgctcagctcacagagcagtcatatctcttggtgtactgggattcttttctagctgtgttgtctgggtttgttcaagaagaaaaccagtcacaagaaaagtgaattacagagactaaatgctgtgaaaaagatcacactttacctccagagtaaaagctagaagtggcgtttgacccctttgcattggattcagatttgcggtgttgtcagaggagtggcagaagtaattttgccattacaaaggtttctgtcaccagtcggattggtatctgctgtctgtgcacccacacagtgtatctgcaacatctgcattgtgccagaagtatcacttaactgatgaactgatcctttatttttctgtaataaaaaggagatatctttgctaacttaagtgcctgtttgctcagaaggttggaggttgtatgctgttcccttgggctgaggagaaccccaaggatgaatttcttgggtgctcattgtcttgagcaggcaagttttgtgtgggtgatctcttttcatggcaggatattaaaatgggaatttgtagtctggaagatggagcagctgtttgtgagactcttgagttagggagagaaatgtataccacgtctgttctcgatccatcagaatggatccatccacctctttgtgtgtggaactgtgtatagtctgtattggttttctacagcacttggatctctttggaccaaattagcgagctgttcattttaacataactgccagtatttatagacaatttcttacggacagataatgaatttagaaactggaggttactttgggcagctgttcctcagctctgtctgtaacttgcaaattattctgagttattttctgcagaacctccttccttatcacgggaggagcctgggagttgaggttgactgtaattgggtcaatggttgtcacagacttaaggtgtccaggctgattggaggaggcactgagccctaacagagcactgagctgacttctaattgcagcatccttgcaaaatgaggaagggagttcagtgatgtctgcactgaagatgtatgatacactgatagcagttctgggtatgttgtaacagcttcaaagtagaaccgcagtactgcgtgagctgtgtgacttcttcctagaacacagcactgtcaccccatatggttgggacgtgcaggtgagaccaacacctaccaggttccctggcgtaccgtggccttctcagttcttgtgccagtgatactgggttctgttctgtggtgtcagacagcgtcctgtagcaaagctgaattcccacttagtctggtgagagaataaagagccatcagccaacagagggagcgttcattctgctggagcagtgcgagctgtaagcattacgagaggcgtagtttcagtttgttgcagtcaggttcctatattttcaaagctgaaatcagaaataagtaaatacggagaaaataagctgttgcttttaatgctctttcctccactaattgtactcttaattttcttcttgggaggccgaggatccatctgcataactttagctgtgatgctccagataagtgtttagaattcattttatctttgactgatgggactgataagaagttaacgcacaatatttttacatacaacatcgttttccagtgacctcctgagcggtgggaagcattatgggatagcaccggctgtgactcgagttcatttgaaggcgatctcttgcctgcaggttaaatgggacggagtcagaatcactgtgagccgtctgtaatcagcaaacagtctgtgggcttttcttactgtgttctctctgtttgccttagtttggtgcaggaagagttccttgtgacagcgtcctttgaggtgtgttgcaggagctgaccatttgctccttgagctgtgtgatgaactgttgtccacttaatggagttacagaagcagcttctgggagtcgcatctggtcgcatacattcagtgttttgggaagctgtcagtgtggtgtttgcactgtgtttgaatggtgttcatggtgggtctgttatgctcctggatgatttggggagatgtggggctgcttccgtggcagacaggatcagctcagggcgctgctgcctatggctgtgggaaacctcacagttggtgtttgaatagtggccaagtatgtcaattaaaaatacattttgaagggaggtttgtcatagctctgtactttggcatgctctgcttactgaaaacatactagctgtagctcaaaaaaagttgtgaatcctcagaataatacaggagctggcaattgtggctgctttctctttgtgttccttttctcttgggttggatgaagctttaaaaaggaaggagccctggtgagggttggtcagtgtgcatttcattcttggaaccagagaggaagttgcatcaactttcaggacgctgcagagctcacttgcacaggtggtgctccagtctatgtgatttttggggtcaaatcttgagatgatcttacaaaatcagattttgtacccatcatgagcatgaggtgagtggttgtgctcggtttctagctgcatgtatgtatacagacacgtgtatgcagacatgtctatgtgtgagtagttcgagtcagtcaaggttactggcagcacctaaagcgtatgcaccacataatgcatgcaggcaaaagtcctatcttaggagccatctcttcatgggtttgggtttatataggcagtatttttaaacagaatatccgaagcactttctggagttctgtggtaatgcagtgacacctatttggatgaaggaagatgtgtctgaggagcacgtaagcagatttgctgccctaacagagaggttttggtaaccgtggaaaaggttttctcctggatctgtgtgtgctcttggtgagctgcaatccatgacagggcacaaccagatgagaaggaaacccggccatcccatgcttgagcacagctctgactcagtagttccaccagatgtgccctttcagtcaaagtgttctgatctcttagagctttctgtagttcaagttaccactcactctccagcttgctcggttaatgtctgttggcggcgttgagttggacttgggaaaggtgtgtgtggtaggaacaagcagagtgtgatgtgcttctgttatcaggacttaagctagagtggttggcagataggaaatgcagctattccttgaaagcaagcagatcatggatggtcagccaaactgccctggctttggtgggagctgcactgcagaaggaccaaaccccaacaagatttggcacatttgtttagaagataagcacagatggttttgcacaaggcagctcctcataatggtggctttgtagatttagtccaaatgttcttatttagatctagcagcacatcactgtgtccgtgcccatctaacctcgctatcctaagtagagcagaccccaaacaaccttgttcaaaaactaccagtgcaaataactgaactaaatatttgttactgctgactgagaacagctgttcgagtgtagcattgtggcttgttaatgtgagtgccccaactctatggtcttattaaagaaacccaaacattgctcagattttgttcttattgtcatcataagacttgaatagtgatggtaatgcttacgtagacgtgtcttgtgagtgcacttcagtgatttagaaagaactggatttcaagcaactttggacctgtggggggagggagattaatgaaggtttgaatcacattctaattctatgtacagtccttcattactccacaagcctaaatcctatacagcctccaggatagctggaaactgttgagatctggactttttttttttaatccaagggctaacttgttgtaacttggtataattatctgctttcggaaatgcatctctgttggtttgaaa

SEQ ID NO: 62 - protein, 189 aa, Gallus gallus
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAQELAKSYGIPFIETSAKTRQGVEDAFYTLVREIRQYRMKKLNSNEDGNQGCMGLSCIVM

SEQ ID NO: 63 - DNA, 2603 bp, Homo sapiens
  source 1..2603: mol_type = genomic DNA
aggcgaggcttccccttccccgcccctcccccggcctccagtccctcccagggccgcttcgcagagcggctaggagcacggcggcggcggcactttccccggcaggagctggagctgggctctggtgcgcgcgcggctgtgccgcccgagccggagggactggttggttgagagagagagaggaagggaatcccgggctgccgaaccgcacgttcagcccgctccgctcctgcagggcagcctttcggctctctgcgcgcgaagccgagtcccgggcgggtggggcgggggtccactgagaccgctaccggcccctcggcgctgacgggaccgcgcggggcgcacccgctgaaggcagccccggggcccgcggcccggacttggtcctgcgcagcgggcgcggggcagcgcagcgggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaattttggtgaatgtgggtagtcattcttacaattgcactgctgttcctgctccatgactggctgtctgcctgtattttcgggattctttgacatttggtggtactttattcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattattttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggatgaaagctaaaaaaaaaaaaaaaaaa

SEQ ID NO: 64 - protein, 393 aa, Homo sapiens [sequence omitted]

SEQ ID NO: 65 - DNA, 2151 bp, Rattus norvegicus
  source 1..2151: mol_type = genomic DNA
cggccgcgcgctccctgctgagttgcaggctgtttcccggctgcaagatgcccaagaagaagccgacgcccatccagctgaacccggcccccgatggctccgcggttaacgggaccagctcggccgagaccaacctggaggccttgcagaagaagctggaggagctggagctggacgagcagcagcggaagcgccttgaggcctttctgacgcagaagcagaaggtgggagagttgaaggatgatgactttgagaagatcagtgaactgggggctggcaatggtggagtggtgttcaaggtctcccacaagccatctggcctggttatggctaggaagctaattcacctggagatcaaacccgcaatccggaaccagatcatccgggagctgcaggtgctgcatgagtgcaactccccgtacatagtgggcttctacggggccttctacagtgacggcgagatcagcatctgcatggagcacatggatggtgggtccttggatcaagtgctgaagaaagctggaagaattcctgagcaaattttaggaaaagtcagcatcgctgtgataaaaggcctgacatatctacgagagaagcacaagattatgcacagagatgtcaagccttccaacattctagtgaactcacgtggggagatcaaactctgcgattttggggtcagcgggcagctaattgactccatggccaactccttcgtgggaacaaggtcctacatgtcgcctgagagactccaggggactcactactctgtgcagtcggacatctggagcatggggctctctctggtggagatggcagttggaagataccccattcctcctcctgatgccaaggagctggagctgctgtttggatgccaggtggaaggagacgcggccgaaacgccacccaggccaaggacccctgggaggcccctcagctcatatggaatggatagccgacctcccatggcaatttttgagttgttggattacatcgtcaatgagcctcctccaaaactgcccagtggagtattcagtctggaatttcaggattttgtgaataagtgcttaataaagaaccctgcagagagagcagatctgaagcagctcatggtacatgctttcatcaagagatctgatgccgaggaggtagacttcgcaggctggctctgctccaccattgggcttaaccagcccagcacaccaacccacgctgccagcatctgagcctttgggaagcagcagagaggaatcctctgcccagtggcatgccatgttgctttcaggcctctcccatgcttgtctatgttcagatgtgcatctcatctgtgacaaaggatgaagaacacagcatgtgccaaatcgtacttgtgtcatttttaatattgtctttatcgctatggttactcccctaagtggattggctttgtgcttggggctatttgtctgttcatcaaatacatgccaggttgaactacagtgaaaccctggtgacctgggtggtcttcttactgatgtttgcgctgctgttcatcgtgactcactagctggctgcctgtattgtcaggattctcggacccttggtacttcactcttgctggtgacctctcagtctgaggagagggggccttctgagacccttcacaggcagtgcatgcatgaaaagcatgctttgctgctactgaaatgagcaccagaacgtgtacatcatggtattttatttttgcttttggtatagaactcagcagttcccatttaaaaaaaaaatctaaccagagcccatcactgccatgatagctggggcttcagtctgtctactgtggtgatttttagacttctggttgtatttctatatttatttttaaatatactgtgtgggatacttagtggtatatgtctctgagtttggattagtgtttctaaattggtagttattttgaatgtcacaaatggattaaggaatcaacgtatcaagagttctatctttcttccagtctaagtaccaatgctattgtaaacgtgtatagtgcctacaaattgtatgaaaacccttttaaccactttactcaagatgtttatcaaatctaatctcttattctaataaaaatactatcaagttaaagtaaaaaaaaaaaaaaaaaaaaaaaaa

SEQ ID NO: 66 - protein, 393 aa, Rattus norvegicus
MPKKKPTPIQLNPAPDGSAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELLFGCQVEGDAAETPPRPRTPGRPLSSYGMDSRPPMAIFELLDYIVNEPPPKLPSGVFSLEFQDFVNKCLIKNPAERADLKQLMVHAFIKRSDAEEVDFAGWLCSTIGLNQPSTPTHAASI

SEQ ID NO: 67 - DNA, 2387 bp, Mus musculus
  source 1..2387: mol_type = genomic DNA
agtccctcactgggacgtctgtgcgcggcgtctcggagcgccggagcagcggtggccgcactttctccaagctggggctgtagctgagctgtgggtagtgcgcagggagccgtccgagcccgaggaaccggtgtgctgaggcgagagttcccggccggcgagcgcgcgcagctggttctccgcgtgggttgggcggagggtcccaggagcgcggcgttgatcgagccgccccgactctgggcagagccgagggaggaagcgagaagcggccgcgcgctccctgctgagttgcaggctctttcccggctgcaagatgcccaagaagaagccgacgcccatccagctgaacccggcccccgatggctcggcggttaacgggaccagctcggccgagaccaacctggaggccttgcagaagaagctggaggagctggagcttgacgagcagcagcggaagcggctcgaggcctttctgacgcagaagcagaaggtgggggaactgaaggatgatgactttgagaagatcagcgaactgggagctggcaacggtggagtggtcttcaaggtctcccacaagccatctggcctggttatggctagaaagctgatccacctggagatcaaacccgcaatccggaaccagatcatccgggagctgcaggtactgcacgagtgcaactccccgtacatcgtgggcttctacggggccttctacagcgacggcgagatcagcatctgcatggagcacatggatggtgggtccttggatcaagttctgaagaaagctggaagaattcctgagcaaattttaggaaaagttagcattgctgtgataaaaggcctgacctatcttcgggagaagcacaagattatgcacagagatgtcaagccatccaacattctagtgaactcacgtggggagatcaaactctgtgattttggggtcagcgggcagctaattgactctatggccaactccttcgtgggcacgagatcctacatgtcgcctgagagactccaggggactcactactctgtgcagtcggacatctggagcatggggctctctctggtggagatggcagttgggagataccccattcctcctcctgatgccaaggagctggagctactgtttggatgccatgtggaaggagacgcagccgaaacaccacccaggccaaggacccctgggaggcctctcagctcatatggaatggacagccgacctcccatggcaatttttgagttgttggattacattgtcaatgagcctcctccaaaactgcccagtggagtattcagtctggagtttcaggattttgtgaataaatgcttaataaagaaccctgcagagagagcagatctgaagcagctcatggtacatgctttcatcaaaagatctgacgccgaggaggtagacttcgcaggctggctctgctccaccattgggcttaaccagcccagcacaccaacccacgctgccagcatctgagcctttaggaagcagcaaagaggaattctctgcccagtggcatgccatgttgctttcaggcctctcccatgcttgtctatgttcagacgtgcatctcatctgtgacaaaggatgaagaacacagcatgtgccaaattgtacttgtgtcatttttaatatcattgtctttatcactatggttactcccctaagtggattggctttgtgcttggggctatttgtctgttcatcaaacacatgccaggctgaactacagtgaaaccctagtgacctgggtggtcgttcttactgatgtttgcactgctgttcatcgtgactcactagctggctgcctgtattgtcaggattctcggaccttggtacttcactcttgctggtgacctctcagtctgagagggagccttgtgagacccttcacaggcagtgcatgcatggaaagcatgctttgctgctactgaaatgagcatcagaacgtgtacgtcatggtatttttattttttgcttttggtatagaactcagcaattcccatcaaaaaaacctaagcagagcccatcactgccatgatagctgggcttcagtctgtctactgtggtgatttttagacttctggttgtatttctatatttatttttaaatatacagtgtgggatacttagtggtgtgtgtctctaagtttggattagtgtttctaaattggtggttattttgaatgtcacaaatggattaaagcatcaatgtatcaagagttctatctttcttccagtctaagtaccaatgctattgtaaacaacgtgtatagtgcctacaaattgtatgaaaccccttttaaccactttaatcaagatgtttatcaaatctaatctcttattctaataaaaatactatcaagtt

SEQ ID NO: 68 - protein, 393 aa, Mus musculus
MPKKKPTPIQLNPAPDGSAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELLFGCHVEGDAAETPPRPRTPGRPLSSYGMDSRPPMAIFELLDYIVNEPPPKLPSGVFSLEFQDFVNKCLIKNPAERADLKQLMVHAFIKRSDAEEVDFAGWLCSTIGLNQPSTPTHAASI

SEQ ID NO: 69 - DNA, 1182 bp, Oryctolagus cuniculus
  source 1..1182: mol_type = genomic DNA
atgccaaagaagaagcccacccccatccagctgaatcctgcccctgacggctcggcggtgaatggtaccagctcggcggagaccaacctggaggccttgcagaagaagctggaggagctggagcttgacgagcagcagcggaagcgcctggaggccttcctcacccagaagcagaaagtgggagagctgaaggacgatgacttcgagaagatcagtgagctgggagccggcaacggcggcgtggtgttcaaggtctcccacaagcccagtggcctggtgatggccagaaagcttattcacctggagatcaaacctgctatccggaaccagatcataagggagctgcaggttctgcacgagtgcaactccccgtacatcgtgggcttctacggggcattctacagcgatggcgagatcagcatctgcatggagcacatggacgggggttccttggatcaagtcctgaagaaagctggacggattcccgagcaaattttggggaaagttagcattgctgtgatcaagggcctgacgtatctgagggagaagcacaagatcatgcacagagatgtgaagccctccaacatcctggtcaactcccgcggggagatcaagctctgtgacttcggggtcagtgggcagctcatcgactccatggccaactccttcgtgggcaccaggtcttatatgtcgcccgagagactccaggggacacactactctgtgcagtcggacatctggagcatggggctgtccctggtggagatggcggtggggcggtaccccatcccgccccccgacgccaaggagctggagctgatgtttgggtgccaggtggagggcgatgcggccgagactccgcccaggcccaggacccctgggcggcccctcagctcgtatggaatggatagccggcctcccatggcgatttttgagctgctggattacatcgtcaatgagcctcctccgaaactccccagcgcagtcttcagcctggagtttcaagattttgtgaataaatgcttaataaaaaaccccgccgagagagcagacttgaagcagctcatggttcatgcttttatcaagaggtctgatgccgaggaggtggattttgctggttggctgtgctccaccatcggccttaaccagcccagcacgccgacgcacgcggccggtgtgtga

SEQ ID NO: 70 - protein, 393 aa, Oryctolagus cuniculus
MPKKKPTPIQLNPAPDGSAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELMFGCQVEGDAAETPPRPRTPGRPLSSYGMDSRPPMAIFELLDYIVNEPPPKLPSAVFSLEFQDFVNKCLIKNPAERADLKQLMVHAFIKRSDAEEVDFAGWLCSTIGLNQPSTPTHAAGV

SEQ ID NO: 71 - DNA, 1872 bp, Cavia porcellus
  source 1..1872: mol_type = genomic DNA
cgtgtcttcgtcgggaccgccctcctccttgagtcctccccccaccgggacggccgagtggagaggccggacgaaggcggcggccccggcggcggcttttcctcggcttcgctgtgcagcgtgcgcggcgaggttgaccgcccgcgagcgcccgtgactgagggaacagggagagagctcgggcggccgagcgcgcagccctccgtgggcattgccgcctggacctcccggaaggcaccccgggccgcggccgccacccgtcccgccctcgttcggagctgagacgccgtcgccgcgcaagatgcccaagaagaagccgacgcccatccagctgaacccggcccccgacggctcggcggtgaacgggaccagctcggccgagaccaacctagaggctttgcagaagaagctggaggagctggagctggatgagcagcagcggaagcgcctcgaagctttcctgacacagaagcagaaggtgggcgagctgaaggacgatgactttgagaagatcagtgagctgggtgccggcaatggcggtgtggtgttcaaggtctcccacaagccatctggcctggtcatggcccgaaagcttatccacctggagatcaagccagccatccgcaatcagatcatccgtgagctgcaggttctgcacgagtgcaactcgccctacattgtgggcttctatggggccttctacagtgatggcgagatcagcatctgcatggagcacatggatggaggttccttggatcaagtcctgaagaaagctggaagaattcctgagcaaattttaggaaaagttagcattgctgtgatcaaaggcctgacatacctgagggagaagcacaagattatgcacagagatgtcaagccctccaacatcctggtcaactcccgcggggagatcaagctctgtgactttggggtcagcgggcagctcatcgattccatggccaactccttcgtgggcacccggtcctacatgtcgccagagagactgcagggcacacactactcagtgcagtcggacatctggagcatgggactgtcactggtggagatggcggttgggaggtaccccatcccccctccagatgccaaggagctggagctggtgttcgggtgccaggtggaaggagatgcagctgagatgccgcccaggcccaggacccccggaagacccctgagctcatatggaatggacagccggcctcccatggcgattttcgagctgttggattacatagtcaacgagccacctcccaaactgcccagtggagtcttcagtctggaattccaggactttgtaaataaatgcttaataaagaaccctgcggagagagcagacttgaagcagctcatggttcatgccttcatcaagcgctctgatgctgaggaggtggacttcgcaggttggctctgtgccaccatcggccttaaccagcccagtaccccgacccacgtggccagcatctgagctgcggcccggcccagacgtgctctgccagcagccgctatgctctggcctctccctcgcttctcttcagacgtgcgtttcacctccgaccagggtgcagacacagcatgtgccaagctgtatttgtgttccttttcagtctttattgccaccgtgtcacccgagtggatttgctttgtgcttagggctgtttgtgctgatgatcacacacacgctgagctgaacagtgacacttggtgatgtggttgtcactgttctcactccatgtggctggcctgttgcctccagtgtctccagacttggggatgtctggtggcacttcccctgccagggcatctcctcagcagagagggaggcctctgggcccttgtccttggcagtgcaagtga

SEQ ID NO: 72 - protein, 393 aa, Cavia porcellus
MPKKKPTPIQLNPAPDGSAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELVFGCQVEGDAAEMPPRPRTPGRPLSSYGMDSRPPMAIFELLDYIVNEPPPKLPSGVFSLEFQDFVNKCLIKNPAERADLKQLMVHAFIKRSDAEEVDFAGWLCATIGLNQPSTPTHVASI

SEQ ID NO: 73 - DNA, 1257 bp, Canis lupus
  source 1..1257: mol_type = genomic DNA
ggagagcgagacacgggccgctctccgctcggagccggacgcgccttcccgcgtccaaaatgcccaagaagaagccgacgcccatccagctgaacccggcccccgacggctcggcggtgaacgggaccagctcggcggagaccaacctggaggccttgcagaagaagctggaggagctggagcttgatgagcagcagcggaagcgccttgaggcctttctcacccagaagcagaaggtcggggaactgaaggatgacgacttcgagaagatcagtgagctgggtgctggcaacggtggcgtggtgttcaaggtctcccacaagccgtccggcctagtcatggccagaaagctaattcacctggagatcaaacctgcaatccggaaccagatcataagggagctacaggttctacatgagtgcaactccccgtacatcgtgggcttctatggtgcattctacagcgatggcgagatcagtatctgcatggagcacatggatgggggttccttggatcaagtcctgaagaaagctggaagaattcctgaacaaattctaggaaaagttagcatcgctgtaataaaaggtctgacatacctgagagagaagcacaagattatgcacagagatgtcaagccttccaacatcctcgtgaactcccgtggggagatcaagctctgtgactttggggtcagcgggcagctcattgactccatggccaactccttcgtgggcacaaggtcctacatgtcgccagaaagactccaggggactcattactccgtgcagtcggacatctggagcatggggctctctctggtggagatggcagttgggaggtatcccatccctcctccggatgccaaggagctggagctgatgtttgggtgccaagtggagggagacgtggctgagaccccacccagaccaaggaccccgggaagaccccttagctcttatggaatggacagccgaccgcccatggcaatttttgagctgttggattacatagtcaacgagccccctccaaaactgcccagtggagtattcagtctggaatttcaagattttgtgaataaatgcttaataaaaaacccagcagagagagcagatctgaagcaactcatggttcatgccttcatcaagagatctgacggtgaagaagtggattttgcaggttggctctgctccccccattggccttaaccagcccagcacgccgacccacgcagctggcgtctaactcgagtctagagat

SEQ ID NO: 74 - protein, 381 aa, Canis lupus
MPKKKPTPIQLNPAPDGSAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELMFGCQVEGDVAETPPRPRTPGRPLSSYGMDSRPPMAIFELLDYIVNEPPPKLPSGVFSLEFQDFVNKCLIKNPAERADLKQLMVHAFIKRSDGEEVDFAGWLCSPHWP

SEQ ID NO: 75 - DNA, 2095 bp, Felis catus
  source 1..2095: mol_type = genomic DNA
agccggcaaggagttgagcgtgcggggtgcataggcgcgggtcgtgggagatgaagctggagaggaccaacctggaggccttgcagaagaagctggaggagctggagctcgatgagcagcaacggaagcgcctggaggcctttcttacccagaagcagaaggtcggggaattgaaggatgacgacttcgagaagatcagcgagctgggcgctggcaacggtggtgtggtgttcaaggtctcccataagccgtctggcctggtcatggccagaaagctaattcacctggagatcaaacctgcaatccggaaccagatcataagggagctgcaggttctacatgagtgcaactccccatacatcgtgggcttctatggcgcgttctacagcgacggcgagatcagtatctgtatggagcacatggatgggggttccttggatcaagtcctgaagaaagctggaagaattcctgaacaaattttaggaaaagttagcattgctgtaataaaaggtctgacatacctgagggagaagcacaagattatgcacagagatgtcaagccttccaacatcctagtgaactctcgtggggagatcaagctctgtgactttggggtcagcgggcagctcatcgactccatggccaactccttcgtgggcacaaggtcctacatgtcgccagaaagactccaggggactcattactccgtgcagtcggacatctggagcatggggctatctctggttgagatggcagtcgggaggtatcccatccctcctcccgatgccaaggagctggagctgatgtttgggtgccaagtggagggagatgcggctgagacgccacccaggccgaggacccccggaaggcccctcagctcgtatggaatggacagccgacctcccatggcaatttttgagttgttggattacatagtcaacgagcctcctccaaagctgcccagtggagtattcagtctggaatttcaagattttgtgaataaatgcctcataaaaaacccagcagagagagcagatctgaaacaactcatggttcatgcctttatcaagagatctgatggtgaggaagtggattttgcaggttggctctgctccaccatcggccttaaccagcccagcacaccgacccacgcggccggcgtctaagtatctgggaagcagcaaagagcgagtcccctgcccagtggtgtgccattgtcgctttcaggcctctttgccatgcctgtctccgttcagacgtgcatttcgcctacgacaaaggatgaagaacacagcatgtgccaaaattctatttgtgtcttttttaatattactgtcatttattctgttatttccctaagtggattggctttgtgcttggggctatttttgtgtatgttgatccaaacatgcgcaacgttcagttacagtgaaaccttggtgactgtgggtagtcattcttactgaaaattgcactgctcttcccccaccgtgactggctagctgcctgtagttttgggattcttttgacacttggtggtactgcattcttgccgggcgcaccttccttctgttggggtaggagccttgtaagatccttcacaggcactgcatgtgaagcatgctttgctgctatgaaaaagaacatcagaaagtatagatcttgttattttattatatttttgcttttggtgtagaatgaagcaatttctgtcaaaatctagccagagcccttcactgccacgatagctggggcttcaccagtctgtctactgtgatgatttgtagacttctggttgtatttctatatttattttaaaatatattatgtgggatatttagtggtatgtgtctctttaagtttgaattagtgtttctaaaatgatggttactttgaatgttacaaatggatcaaggcattaaaatgtatgagatttatctttccccaaatccaagtaccgatgctattgtaaacaacagtgtgtatagtgcctaagaattgtatgaaaatccttttaaccatttcaacccagatgtttaacaaatctaatctcttattctaataaatatactatcaagttaaaaggatgaaaaaa

SEQ ID NO: 76 - protein, 371 aa, Felis catus
MKLERTNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELMFGCQVEGDAAETPPRPRTPGRPLSSYGMDSRPPMAIFELLDYIVNEPPPKLPSGVFSLEFQDFVNKCLIKNPAERADLKQLMVHAFIKRSDGEEVDFAGWLCSTIGLNQPSTPTHAAGV

SEQ ID NO: 77 - DNA, 2405 bp, Bos taurus
  source 1..2405: mol_type = genomic DNA
ccggttgactgagggagagtgggagggaatcccgggctgccgagctgcgccggcggggaagcccttcggttccctgtgcactgagcaagtgggccggggggttcccccagaccgccactggccctttggccctgacgggaccgcgcagggcgcgccccccgaaggcagccttcgggcttgcggcccagacttggccccgcgaggccggcgcggggcagctcagagggaggaagctagaggggccgccctcagagttgggagcgcctttcctgggtccaaaatgcccaagaagaagccgacgcccatccagctgaacccggccccggacggctccgcggttaacgggaccagctcggcggagaccaacctggaggccttgcagaagaagctggaggagctggagctcgatgaacagcagcggaagcgcctcgaggcctttctgacccagaagcagaaggtgggggaactgaaggatgatgactttgagaagatcagtgagctgggtgccggcaatggaggtgtggtgttcaaggtctcccacaagccgtccggacttgttatggccagaaagctaattcacctggagatcaaacctgccatccggaaccagatcataagggagctgcaggttctccatgagtgcaactcgccttatatcgtgggcttctacggggcgttctacagcgacggcgagatcagcatctgcatggagcacatggatgggggttccttggatcaagttctgaagaaagctggaagaattcctgaacaaattttaggaaaagttagcattgctgtaataaaaggcctgacatacctgagggagaagcacaagattatgcacagagatgtcaagccgtccaacatcctagtgaacagccgtggagagatcaagctctgtgactttggggtcagcgggcagctcatcgactccatggccaactccttcgtgggcaccaggtcctacatgtcgccagagcgactccaggggacccattactccgtgcagtcggacatctggagcatggggctctctctggttgagatggctgtcgggaggtatcccatccctcctccagatgccaaggagctggagctgatgtttgggtgccaggtggagggagatgcggctgagaccccgcccaggccaaggacccccgggaggcccctcagctcttatggaatggacagccgacctccaatggcaatttttgagttgttggattacatagtcaatgagcctcctccaaaactgcccagtggagtattcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccaacccagcacacccacccatgcggctggcgtctaagtggttgggaagcagcagtccctgcccaagggcatgcactgttgcttccgggcagccttcccatgcctgtctctgttcagacgtgcatttcacctatgacaaaggatgaagaacacagcatgtgccaaaattctatttgtgtcattttcaatattatcatctttactcttattactattgttattcccctaagtggattggctttgtgcttggggctatttttgtgtatattgatgatgaagacatgtgcaatgtagaattacagtgaaactctggtgactgtgggtagtcattcttactgaaaactgcactgctttcccacaccatgaactggctggtcgcctctattttcgggattctttgacacttggtggtacttcattcttgccaggcataccttctaactgagtaggaaggagccttgtaagatccttcacaggcagtgcatgtgaagcatgctttgctgctataaaaatgagcatcagaaagtgtgtatcatgttattttattatgttcttgcttttggtgtagaattcagcaaattttcatcaaaatctagccagagcccttcactgccatgatagctggggcttcaccagtctgtctactgtgatgatttgtagacttctggttgtatttctgtatttatttttaaatctaccgtgtggatatttagtgctatgtctctttaagtttggattagtgtttctaaaatggtggagttgctctgaatgttacaaatggatcaaggcattaaaatgaatgagatctacctttcaccaagtactgatgctattgtaaacaacagtgtgtatagtgcctaacaactgtatgaaaatccttttaccattttaatccagatgtttaacaagcctaatctcttactctaataaatatactatcaaattcaaaggaaaaaaaaaaaaaaaaaaaaaaaaaaaaa

SEQ ID NO: 78 - protein, 393 aa, Bos taurus [sequence omitted]

SEQ ID NO: 79 - DNA, 2146 bp, Equus caballus
  source 1..2146: mol_type = genomic DNA
caagtgggaaagcttgggatgcgtggaggagccagctactagtttagtgtgctgggtgcgtcggtgcaggtcgcaggagatgaagccggagaggaccaacctggaggccttgcagaagaagctggaggagctggagctcgatgaacagcagcgaaagcgccttgaggcctttcttactcagaagcagaaggttggggaactgaaggatgatgactttgagaagatcagtgagctgggtgctggcaatggtggtgtggtattcaaggttgcccacaaaccgtctggtttggtcatggccagaaagctaattcacctggagatcaagcctgcaatccggaaccagatcataagggagctgcaggttctacatgagtgcaactccccatatattgtgggcttctatggcgcattctacagcgatggtgagatcagcatctgcatggagcacatggatgggggttccttggatcaagtcctaaagaaagctggaagaattcctgagcaaattttaggaaaagttagcattgctgtaataaaaggcctgacgtatctgagggagaagcacaagattatgcacagagatgtcaagccctccaacatcctagtgaactcccgtggggagatcaagctgtgtgattttggggtcagcgggcagctcatcgactccatggccaactccttcgtgggcacaaggtcttacatgtcgccggaaagactccaggggactcattattcagtgcagtcggacatctggagcatggggctctctctggttgagatggcggtcgggaggtatcccatccctcctccagatgccaaggagctggagctgatgtttgggtgccaagtggagggagatgcggctgagactccgcctaggccaaggacccctggaagacccctcagctcttatggaatggacagccgacctcctatggcaatttttgagttactggattacatagtcaacgagcctcctcccaagctgcccagtggagtattcagtctggaatttcaggattttgtgaataaatgcttaatcaaaaaccctgcagagagagcagatttgaagcaactcatggttcacgcttttatcaagagatctgatgccgaggaagtggattttgcaggttggctctgctccaccattggccttaaccagcccagcacaccaacccacgcggctggcgtctaagcgtttgggaagcagcaaaaagcgagccccctgccgcgtggtgtgccatgttgctttcgggcctccttcccatgcctgtctgttcacacgtgcatttcacctgtgacaaaggatgaagaacacagcatgtgccaaaattctatttgtgtcatttttaatagtactgtctttattcttattactattgttattcccctaagtggattggctttgtgcttgggactattttgtgtatgttgatgatcaaaacatgcgcaatgttgaattaccgtgaaactggtgactgtgggtagtccttcttattgaaaattgcactgctcttccctccctgtcactggctggctgcctgtatttctggggttctttgacacttggtggtacttcattcttgcagggcatacctcctattcgagtaggaaggagcctttaagatccttcacaggcagtgcatgtgaagcatgctttgctgctatgaaaatgagcatcagaaagtgtatatcatgttattttattattattatgtttttgcttttggtgtagaattcagcaatttccatcaagatctagccagagcccttcactgccatgatagctggggcttcaccagtctgcctactgtgatgatttgtagacttctggttgtatttctatatttatttttaaatatactgtgtgggatatttagtggtatatgtctctctaagtttggagtggtgtttctaaaatggagttactttgaatgttatagatggatcaaggcataaaatgtatgagatttatttttccccaaatccaaatactgatgctattgtaaacaacaaacagtgtgtatagtgcctaaaaattgtatgaaagtccttttaaccattttaatccagatgtttaacaaatctaatctcttattctaataaatatactatcaagttaaacggacaaaagatttctactttc

SEQ ID NO: 80 - protein, 371 aa, Equus caballus
MKPERTNLEALQKKLEELELDEQQRKRLEAFLTQKQKVGELKDDDFEKISELGAGNGGVVFKVAHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELMFGCQVEGDAAETPPRPRTPGRPLSSYGMDSRPPMAIFELLDYIVNEPPPKLPSGVFSLEFQDFVNKCLIKNPAERADLKQLMVHAFIKRSDAEEVDFAGWLCSTIGLNQPSTPTHAAGV

SEQ ID NO: 81 - DNA, 2274 bp, Gallus gallus
  source 1..2274: mol_type = genomic DNA
ctaaccaggcgggagctgtcggtgcggagctcggtgtcgctccgccgggcaggccgggtcgacggccgcgctgtgccggagcggcagcgtcgcgggctcggctccttctcggggaggcggccgcgcgctgctccggcgctgaggggcggccccgaagtttgcttcgcgtcgggaagtccgtcggacctggccgaagtggggccgcggccgctccgtccgtcacgctctgcgctggccggggggcaacatgcccaagaagaagccagggccgatccagctcaaccccgctccggatggctccgccgtcaacgggaccagctctgccgagacaaacttggaagctcttcagaagaagctggaagagctagagctggatgaacagcaaaggaagcgccttgaagctttccttacccagaaacaaaaagttggggagctgaaggatgatgacttcgagaagatcagtgagctgggagcagggaatggcggggtggtgttcaaagtatctcacaagccttctggcctcatcatggcaagaaaattgattcatctagaaatcaagccagctattcgaaaccagatcatccgtgagctgcaggttctacatgagtgcaattcaccatacatagtgggcttttatggagctttttacagtgatggggaaatcagcatttgcatggaacacatggatggtggctcattggatcaagtgctgaaaaaggctggaagaattccagagcagatactgggcaaagtcagcattgcagtaataaaaggactcacatatctgagagaaaagcataaaataatgcacagagatgttaaaccatctaacatcttggtaaactctagaggtgagatcaagctttgtgattttggtgtcagtggacaactgatagattctatggcaaactcatttgttggcacgcgctcctacatgtctccggaaagactgcagggaactcattattcagtgcagtcagatatatggagtatggggctgtctctggtagaaatggccattggcagatacccgattcctcctcctgactctaaggagctcgagttgatgtttggctgcccggtagagggagattctccagtcacagagacctcacccaggcaaagaacacctggtcgaccaatgagctcctatggaccagacagcagacccccgatggcaatctttgaacttctggattacatcgtcaatgagccacctccaaaactgcccaatggtgtctttggttctgaatttcaagattttgttaacaaatgtttaattaaaaatcctgctgagagagctgatttgaagcagctgatgattcacgctttcattaagagatctgaagcagaggaggtggattttgcaggatggctttgctcaaccataggccttaaccaaccgagtacacccacgcatgctgctggagtctgaatgtggaagagcaaatcctgtcccgtacatctgttaacagcgctactttggtcctatttcctaagcttgtacctgttcaaacatgtatttcacctcttaaggaagaatgtctttatagcatgtgccaaattgttttcaattttgtcatcaactaattggtattgtactgggttacatttgtttgctgaccaaaatgtaaaatgtttaagttacagtgcttgctgattttaagtgattatggaattatggatattctttcttaatgaaaatatcactgggggggaatttacccctggattgtttgaactttatcaagactctttgtaaactgttggtacttcagtcatgcttacctaatctcccatgcaaaaaaaggggtagggatgctccaaaactgtatctgttgagcatgcttttgctgctgccaaactgtatcttggaagttaggcctaatggttccaatttggtgttgtgtagagatcactctttccaggtaaagaaggtaagagctctgcattccttgggatggacagggcagtatcctacttgtagacttgttcatatttctatatttatttttaaaatgtatcatcatacttggatttagtgatatatgtctttccaattgatttttaaaggttagctctcagaagcgtcctacagaatcatgacaaagatctgggctttcttttaaccttaagattcatgacagctgtgtttggtgtctaaaatgtatgaagatcctctattgttttattctctcagatgtttagcaatggtttctcttaataaatatattatcaagtaaaaaaaaaaaaaaaataaaaaaaaaaaaaaaa

SEQ ID NO: 82 - protein, 395 aa, Gallus gallus
MPKKKPGPIQLNPAPDGSAVNGTSSAETNLEALQKKLEELELDEQQRKRLEAFLTQKQKVGELKDDDFEKISELGAGNGGVVFKVSHKPSGLIMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAIGRYPIPPPDSKELELMFGCPVEGDSPVTETSPRQRTPGRPMSSYGPDSRPPMAIFELLDYIVNEPPPKLPNGVFGSEFQDFVNKCLIKNPAERADLKQLMIHAFIKRSEAEEVDFAGWLCSTIGLNQPSTPTHAAGV

(19) United States
(12) Patent Application Publication
(10) Pub. No.: US 2026/0224546 A1
(43) Pub. Date: Aug. 6, 2026

(54) METHODS AND COMPOSITIONS FOR TREATING NON-ERK MAPK PATHWAY INHIBITOR-RESISTANT CANCERS

(71) Applicant: BIOMED VALLEY DISCOVERIES, INC., Kansas City, MO (US)

(72) Inventors: Saurabh Saha, Wellesley Hills, MA (US); Dean Welsch, Parkville, MO (US); Gary Decrescenzo, Parkville, MO (US); Jeffrey Roix, Boston, MA (US)

(21) Appl. No.: 19/557,049

(22) Filed: Mar. 4, 2026

Related U.S. Application Data
(63) Continuation of application No. 17/561,076, filed on Dec. 23, 2021, now Pat. No. 12,599,593.
Application No. 17/561,076 is a continuation of application No. 16/302,955, filed on May 22, 2017, now Pat. No. 11,246,859.
Application No. 16/302,955 is a continuation of application No. 15/161,137, filed on May 20, 2016, now abandoned.
Application No. 15/161,137 is a continuation-in-part of application No. PCT/US14/71749, filed on Dec. 19, 2014.
(60) Provisional application No. 61/919,551, filed on Dec. 20, 2013.

(51) Int. Cl. A61K 31/4439; A61K 31/506; A61K 31/519; A61K 45/06; C12Q 1/6886; G01N 33/575

(52) U.S. Cl. CPC A61K 31/4439; A61K 31/506; A61K 31/519; A61K 45/06; C12Q 1/6886; G01N 33/5758; C12Q 2600/106; C12Q 2600/156

(57) ABSTRACT

The present invention provides, inter alia, methods, pharmaceutical compositions, and kits for treating or ameliorating the effects of a cancer in a subject, which cancer is refractory or resistant to non-ERK MAPK pathway inhibitor therapy. Also provided are methods for identifying a subject having cancer who would benefit from therapy with an ERK inhibitor and methods for inhibiting phosphorylation of RSK in a cancer cell that is refractory or resistant to a non-ERK MAPK pathway inhibitor.

### CROSS REFERENCE TO RELATED APPLICATIONS

The present application is a continuation of U.S. patent application Ser. No. 17/561,076, file Dec. 23, 2021, which is a continuation of U.S. patent application Ser. No. 16/302,955, file Nov. 19, 2018, now U.S. Pat. No. 11,246,859, which is the National Stage of International Application No. PCT/US2017/033843, filed May 22, 2017, which claims benefit to U.S. patent application Ser. No. 15/161,137, filed on May 20, 2016, which is a continuation in part of PCT international application no. PCT/US2014/071749, filed on Dec. 19, 2014, which claims benefit of U.S. Patent Application Ser. No. 61/919,551, filed on Dec. 20, 2013, all of which are incorporated by reference in their entireties as if recited in full herein.

### FIELD OF INVENTION

The present invention provides, inter alia, methods, pharmaceutical compositions, and kits for treating or ameliorating the effects of a cancer in a subject, which cancer is refractory or resistant to non-ERK MAPK pathway inhibitor therapy.

### INCORPORATION BY REFERENCE OF SEQUENCE LISTING

This application contains references to amino acids and/or nucleic acid sequences that have been filed concurrently herewith as sequence listing XML file “1065272.000740-seq-2.xml”, file size of 225,250 bytes, created on Apr. 10, 2026. The aforementioned sequence listing is hereby incorporated by reference in its entirety pursuant to 37 C.F.R. § 1.52(e)(5).

### BACKGROUND OF THE INVENTION

Drug inhibitors that target components of the mitogen-activated protein kinases (MAPK) signaling pathway show clinical efficacy in a variety of cancers, particularly those bearing mutations in the BRAF protein kinase. Both RAF and MEK inhibitors are approved for single-agent use in advanced metastatic BRAF mutant melanoma. Either alone or in combination, BRAF and MEK inhibitor activity is unpredictable in other cancers, with promising efficacy in BRAF mutant thyroid and lung cancer, but only marginal activity in BRAF mutant colorectal cancer.

As with other targeted therapies, patterns of disease response to RAF and MEK inhibitors appear to be influenced by the intrinsic genetic heterogeneity present in the cancers where the drugs are used. For instance, it has been shown that certain genetic alterations, including PTEN and other changes that activate the PI3K cell growth signaling pathway, may predict a poor initial response, and/or relatively rapid progression, in BRAF mutant melanoma treated with the RAF inhibitor vemurafenib. Likewise, direct mutations in MEK gene loci appear to emerge in tumors that have progressed following either BRAF, MEK, or combined drug treatment. Several additional examples, from RAS and RAF gene amplification and splicing mutations, suggest that acquired drug resistance is produced when oncogenic pleiotropy encounters the selective pressure of targeted drug treatment.

In view of the foregoing, there is a need for novel targeted agents that would ideally inhibit diverse nodes of oncogenic pathways, and also be effective in combinations by inducing a burden of selective pressures that exceeds the adaptive capacity of diverse cancer genomes. The present application is directed to meeting these and other needs.

### SUMMARY OF THE INVENTION

One embodiment of the present invention is a method for treating or ameliorating the effects of a cancer in a subject, which cancer is refractory or resistant to non-ERK MAPK pathway inhibitor therapy. The method comprises administering to the subject an effective amount of BVD-523 or a pharmaceutically acceptable salt thereof.

Another embodiment of the present invention is a method for treating or ameliorating the effects of a cancer in a subject. The method comprises:
(a) identifying a subject with cancer that has become refractory or resistant to BRAF inhibitor therapy, MEK inhibitor therapy, or BRAF and MEK inhibitor therapy; and
(b) administering to the subject with said refractory or resistant cancer an effective amount of an ERK inhibitor, which is BVD-523 or a pharmaceutically acceptable salt thereof.

A further embodiment of the present invention is a method for treating or ameliorating the effects of cancer in a subject, which cancer is refractory or resistant to BRAF inhibitor therapy, MEK inhibitor therapy, or both. The method comprises administering to the subject an effective amount of BVD-523 or a pharmaceutically acceptable salt thereof.

Another embodiment of the present invention is a method for identifying a subject having cancer who would benefit from therapy with an ERK inhibitor. The method comprises:
(a) obtaining a biological sample from the subject; and
(b) screening the sample to determine whether the subject has one or more of the following markers:
   (i) a switch between RAF isoforms,
   (ii) upregulation of receptor tyrosine kinase (RTK) or NRAS signaling,
   (iii) reactivation of mitogen activated protein kinase (MAPK) signaling,
   (iv) the presence of a MEK activating mutation,
   (v) amplification of mutant BRAF,
   (vi) STAT3 upregulation,
   (vii) mutations in the allosteric pocket of MEK that directly block binding of inhibitors to MEK or lead to constitutive MEK activity,
      wherein the presence of one or more of the markers confirms that the subject's cancer is refractory or resistant to BRAF and/or MEK inhibitor therapy and that the subject would benefit from therapy with an ERK inhibitor, which is BVD-523 or a pharmaceutically acceptable salt thereof.

A further embodiment of the present invention is a pharmaceutical composition for treating or ameliorating the effects of a cancer in a subject, which cancer is refractory or resistant to non-ERK MAPK pathway therapy. The composition comprises a pharmaceutically acceptable carrier or diluent and an effective amount of BVD-523 or a pharmaceutically acceptable salt thereof.

Another embodiment of the present invention is a kit for treating or ameliorating the effects of a cancer in a subject, which cancer is refractory or resistant to non-ERK MAPK pathway therapy. The kit comprises any of the pharmaceutical compositions according to the present invention packaged together with instructions for its use.

Another embodiment of the present invention is a method for inhibiting phosphorylation of RSK in a cancer cell that is refractory or resistant to a non-ERK MAPK pathway inhibitor. The method comprises contacting the cancer cell with an effective amount of BVD-523 or a pharmaceutically acceptable salt thereof for a period of time sufficient for phosphorylation of RSK in the cancer cell to be inhibited.

Another embodiment of the present invention is a method of treating a subject having an unresectable or metastatic BRAF600 mutation-positive melanoma comprising administering to the subject 600 mg BID of BVD-523 or a pharmaceutically acceptable salt thereof.

Another embodiment of the present invention is a composition for treating a subject having an unresectable or metastatic BRAF600 mutation-positive melanoma, the composition comprising 600 mg of BVD-523 or a pharmaceutically acceptable salt thereof and optionally a pharmaceutically acceptable carrier, adjuvant, or vehicle.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1C show the progress of a dose escalation study in a human malignant melanoma cell line (A375 cells) for month 1. Various treatments (trametinib (a type 2 MEK inhibitor), dabrafenib (a BRAF inhibitor), and BVD-523 (an ERK1/2 inhibitor)) are as labeled.

FIG. 2A-FIG. 2H show the results of a proliferation assay that tracks changes in sensitivity to the escalated agent(s) at month 1. Various treatments (trametinib, dabrafenib, BVD-523, and paclitaxel) are as labeled on the top of the graph. The caption to the right of the graph shows the various types of cells generated from the dose escalation study. For example, “dabrafenib” refers to the cells that have been treated with the highest dose of dabrafenib from month 1 of the dose escalation study. Parental refers to the control cells that have not been treated with drugs. FIG. 2A, FIG. 2C and FIG. 2G are normalized to control, whereas FIG. 2D, FIG. 2F and FIG. 2H show the raw data.

FIG. 3A-FIG. 3D show the progress of a dose escalation study in A375 cells for month 2. Various treatments (trametinib, dabrafenib, and BVD-523) are as labeled.

FIG. 4A-FIG. 4H show the results of a proliferation assay that tracks changes in sensitivity to the escalated agent(s) at month 2. Various treatments (trametinib, dabrafenib, BVD-523, and paclitaxel) are as labeled on the top of the graph. The caption to the right of the graph shows the various types of cells generated from the dose escalation study. For example, “dabrafenib” refers to the cells that have been treated with the highest dose of dabrafenib from month 2 of the dose escalation study. Parental refers to the control cells that have not been treated with drugs. FIG. 4A, FIG. 4C and FIG. 4G are normalized to control, whereas FIG. 4D, FIG. 4F and FIG. 4H show the raw data.

FIG. 5A-FIG. 5H show only the parental and BVD-523 cell line data from FIG. 4A-FIG. 4H. Various treatments (trametinib, dabrafenib, BVD-523, and paclitaxel) are as labeled. FIG. 5A, FIG. 5C and FIG. 5G are normalized to control, whereas FIG. 5D, FIG. 5F and FIG. 5H show the raw data.

FIG. 6A-FIG. 6D show the progress of the dose escalation study in a human malignant cell line (A375 cells) for month 3. Various treatments (trametinib, dabrafenib, and BVD-523) are as labeled.

FIG. 7 is a histogram showing the results of a proliferation assay as applied to cells grown in the DMSO control wells from the dose escalation assay.

FIG. 8A-FIG. 8D are a set of line graphs showing proliferation assays for month 3 of the study. Various treatments (trametinib, dabrafenib, BVD-523, and paclitaxel) are as labeled on the top of the graph. The caption to the right of the graph shows the various types of cells generated from the dose escalation study. For example, “dabrafenib” refers to the cells that have been treated with the highest dose of dabrafenib from month 3 of the dose escalation study. Parental refers to the control cells that have not been treated with drugs.

FIG. 9A-FIG. 9D show only the parental, dabrafenib, and BVD-523 cell line data from FIG. 8A-FIG. 8D.

FIG. 10A is a dose matrix showing % inhibition of the trametinib/dabrafenib combination in A375 cells using the Alamar Blue cell viability assay. FIG. 10B is a dose matrix showing excess over Bliss for the trametinib/dabrafenib combination. FIG. 10C and FIG. 10D show % viability relative to DMSO only treated controls for dabrafenib and trametinib single agent treatments in A375 cells using the Alamar Blue cell viability assay. FIG. 10E shows % viability relative to DMSO only treated controls for dabrafenib and trametinib combination treatments in A375 cells using the Alamar Blue cell viability assay.

FIG. 11A is a dose matrix showing % inhibition of the trametinib/dabrafenib combination in A375 cells using the CellTiter-Glo cell viability assay. FIG. 11B is a dose matrix showing excess over Bliss for the trametinib/dabrafenib combination. FIG. 11C and FIG. 11D show % viability relative to DMSO only treated controls for dabrafenib and trametinib single agent treatments in A375 cells using the CellTiter-Glo cell viability assay. FIG. 11E shows % viability relative to DMSO only treated controls for dabrafenib and trametinib combination treatments in A375 cells using the CellTiter-Glo cell viability assay.

FIG. 12A is a dose matrix showing % inhibition of the BVD-523/dabrafenib combination in A375 cells using the Alamar Blue cell viability assay. FIG. 12B is a dose matrix showing excess over Bliss for the BVD-523/dabrafenib combination. FIG. 12C and FIG. 12D show % viability relative to DMSO only treated controls for dabrafenib and BVD-523 single agent treatments in A375 cells using the Alamar Blue cell viability assay. FIG. 12E shows % viability relative to DMSO only treated controls for dabrafenib and BVD-523 combination treatments in A375 cells using the Alamar Blue cell viability assay.

FIG. 13A is a dose matrix showing % inhibition of the BVD-523/dabrafenib combination in A375 cells using the CellTiter-Glo cell viability assay. FIG. 13B is a dose matrix showing excess over Bliss for the BVD-523/dabrafenib combination. FIG. 13C and FIG. 13D show % viability relative to DMSO only treated controls for dabrafenib and BVD-523 single agent treatments in A375 cells using the CellTiter-Glo cell viability assay. FIG. 13E shows % viability relative to DMSO only treated controls for dabrafenib and BVD-523 combination treatments in A375 cells using the CellTiter-Glo cell viability assay.

FIG. 14A is a dose matrix showing % inhibition of the trametinib/BVD-523 combination in A375 cells using the Alamar Blue cell viability assay. FIG. 14B is a dose matrix showing excess over Bliss for the trametinib/BVD-523 combination. FIG. 14C and FIG. 14D show % viability relative to DMSO only treated controls for BVD-523 and trametinib single agent treatments in A375 cells using the Alamar Blue cell viability assay. FIG. 14E shows % viability relative to DMSO only treated controls for BVD-523 and trametinib combination treatments in A375 cells using the Alamar Blue cell viability assay.

FIG. 15A is a dose matrix showing % inhibition of the trametinib/BVD-523 combination in A375 cells using the CellTiter-Glo cell viability assay. FIG. 15B is a dose matrix showing excess over Bliss for the trametinib/BVD-523 combination. FIG. 15C and FIG. 15D show % viability relative to DMSO only treated controls for BVD-523 and trametinib single agent treatments in A375 cells using the CellTiter-Glo cell viability assay. FIG. 15E shows % viability relative to DMSO only treated controls for BVD-523 and trametinib combination treatments in A375 cells using the CellTiter-Glo cell viability assay.

FIG. 16A-FIG. 16D are a set of images showing Western blot analysis of MAPK signaling in A375 cells after a 4 hour treatment with various concentrations (in nM) of BVD-523, dabrafenib (Dab), and Trametinib (Tram). 40 μg of total protein was loaded in each lane except where indicated otherwise. In this experiment, duplicate samples were collected. FIG. 16A and FIG. 16B show results from duplicate samples. Similarly, FIG. 16C and FIG. 16D also show results from duplicate samples. In FIG. 16A and FIG. 16B, pRSK1 had a relatively weak signal in A375 cells compared to other markers. A different pRSK1-S380 antibody from Cell Signaling (cat. #11989) was tested but did not give a detectable signal (data not shown). In FIG. 16C and FIG. 16D, pCRAF-338 gave a minimal signal.

FIG. 17A-FIG. 17D are a set of images showing Western blot analysis of MAPK signaling in a human colorectal carcinoma cell line (HCT116 cells) after a 4 hour treatment with various concentrations (in nM) of BVD-523, dabrafenib (Dab), and Trametinib (Tram). 40 μg of total protein was loaded in each lane except where indicated otherwise. In this experiment, duplicate samples were collected. FIG. 17A and FIG. 17B show results from duplicate samples. Similarly, FIG. 17C and FIG. 17D also show results from duplicate samples. In FIG. 17A and FIG. 17B, pRSK1 levels appear to be very low in HCT116 cells, and in FIG. 17C and FIG. 17D, pCRAF-338 signal was also very weak.

FIG. 18A-FIG. 18D are a set of images showing Western blot analysis of cell cycle and apoptosis signaling in A375 melanoma cells after a 24 hour treatment with various concentrations (in nM) of BVD-523 (“BVD523”), trametinib (“tram”) and/or dabrafenib (“Dab”) as labelled. 50 μg of total protein was loaded in each lane except where indicated otherwise. In this experiment, duplicate samples were collected. FIG. 18A and FIG. 18B show results from duplicate samples. Similarly, FIG. 18C and FIG. 18D also show results from duplicate samples. In FIG. 18A and FIG. 18B, no band of a size corresponding to cleaved PARP (89 kDa) was apparent.

FIG. 19 shows that BVD-523 can treat acquired resistance to targeted drugs in-vivo. A patient-derived line, ST052C, was isolated from a BRAFV600E melanoma patient that progressed following 10 months of therapy with MAPK-pathway directed therapies. Treated ex vivo, ST052C exhibited acquired cross-resistance to dabrafenib at 50 mg/kg BID. Meanwhile, BVD-523 was effective in ST052C as a single-agent at 100 mg/kg BID.

FIG. 20 is a flowchart showing the dose escalation protocol used herein.

FIG. 21 shows a schematic of the mitogen-activated protein kinases (MAPK) pathway.

FIG. 22A-FIG. 22E show the results of single agent proliferation assays. Proliferation results are shown for treatment with BVD-523 (FIG. 22A), SCH772984 (FIG. 22B), Dabrafenib (FIG. 22C), Trametinib (FIG. 22D), and Paclitaxel (FIG. 22E).

FIG. 23A-FIG. 23O show the results of the combination of BVD-523 and Dabrafenib. FIG. 23A shows a dose matrix showing inhibition (%) for the combination in RKO parental cells. FIG. 23B-FIG. 23C show the results of single agent proliferation assays for the combination in FIG. 23A. FIG. 23D shows Loewe excess for the combination in FIG. 23A and FIG. 23E shows Bliss excess for the combination in FIG. 23A. FIG. 23F shows a dose matrix showing inhibition (%) for the combination in RKO MEK1 (Q56P/+)−clone 1 cells. FIG. 23G-FIG. 23H show the results of single agent proliferation assays for the combination in FIG. 23F. FIG. 23I shows Loewe excess for the combination in FIG. 23F and FIG. 23J shows Bliss excess for the combination in FIG. 23F. FIG. 23K shows a dose matrix showing inhibition (%) for the combination in RKO MEK1 (Q56P/+)−clone 2 cells. FIG. 23L-FIG. 23M show the results of single agent proliferation assays for the combination in FIG. 23K. FIG. 23N shows Loewe excess for the combination in FIG. 23K and FIG. 23O shows Bliss excess for the combination in FIG. 23K.

FIG. 24A-FIG. 24O show the results of the combination of SCH772984 and Dabrafenib. FIG. 24A shows a dose matrix showing inhibition (%) for the combination in RKO parental cells. FIG. 24B-FIG. 24C show the results of single agent proliferation assays for the combination in FIG. 24A. FIG. 24D shows Loewe excess for the combination in FIG. 24A and FIG. 24E shows Bliss excess for the combination in FIG. 24A. FIG. 24F shows a dose matrix showing inhibition (%) for the combination in RKO MEK1 (Q56P/+)−clone 1 cells. FIG. 24G-FIG. 24H show the results of single agent proliferation assays for the combination in FIG. 24F. FIG. 24I shows Loewe excess for the combination in FIG. 24F and FIG. 24J shows Bliss excess for the combination in FIG. 24F. FIG. 24K shows a dose matrix showing inhibition (%) for the combination in RKO MEK1 (Q56P/+)−clone 2 cells. FIG. 24L-FIG. 24M show the results of single agent proliferation assays for the combination in FIG. 24K. FIG. 24N shows Loewe excess for the combination in FIG. 24K and FIG. 24O shows Bliss excess for the combination in FIG. 24K.

FIG. 25A-FIG. 25O show the results of the combination of Trametinib and Dabrafenib. FIG. 25A shows a dose matrix showing inhibition (%) for the combination in RKO parental cells. FIG. 25B-FIG. 25C show the results of single agent proliferation assays for the combination in FIG. 25A. FIG. 25D shows Loewe excess for the combination in FIG. 25A and FIG. 25E shows Bliss excess for the combination in FIG. 25A. FIG. 25F shows a dose matrix showing inhibition (%) for the combination in RKO MEK1 (Q56P/+)−clone 1 cells. FIG. 25G-FIG. 25H show the results of single agent proliferation assays for the combination in FIG. 25F. FIG. 25l shows Loewe excess for the combination in FIG. 25F and FIG. 25J shows Bliss excess for the combination in FIG. 25F. FIG. 25K shows a dose matrix showing inhibition (%) for the combination in RKO MEK1 (Q56P/+)−clone 2 cells. FIG. 25L-FIG. 25M show the results of single agent proliferation assays for the combination in FIG. 25K. FIG. 25N shows Loewe excess for the combination in FIG. 25K and FIG. 25O shows Bliss excess for the combination in FIG. 25K.

FIG. 26A shows Lowe Volumes for the combinations tested. FIG. 26B shows Bliss Volumes for the combinations tested. FIG. 26C shows Synergy Scores for the combinations tested.

FIG. 27A-FIG. 27I show the changes in MAPK and Effector Pathway Signaling in MEK acquired resistance. Isogenic RKO parental and MEK1 (Q56P/+) cells were treated with compound for 4 or 24 h and then immuno-blotted with the indicated antibodies. Dabrafenib was the BRAF inhibitor and trametinib was the MEK inhibitor. FIG. 27A shows increased signaling in RKO MEK1 (Q56P/+) cells. FIG. 27B-FIG. 27C show the results of a 4 hour treatment in Experiment 1 (See, Example 7) in RKO Parental (27B) and RKO MEK1 (Q56P/+) (27C) cells. FIG. 27D-FIG. 27E show the results of a 4 hour treatment in Experiment 2 (See, Example 7) in RKO Parental (27D) and RKO MEK1 (Q56P/+) (27E) cells. FIG. 27F-FIG. 27G show the results of a 4 hour treatment in Experiment 2 (See, Example 7) in RKO Parental (27F) and RKO MEK1 (Q56P/+) (27G) cells. FIG. 27H-FIG. 27I show a summary of results in RKO Parental (27H) and RKO MEK1 (Q56P/+) (27I) cells.

FIG. 28A-FIG. 28E show the results of the combination of BVD-523 and SCH772984. FIG. 28A shows a dose matrix showing inhibition (%) for the combination in A375 cells. FIG. 28B-FIG. 28C show the results of single agent proliferation assays for the combination in FIG. 28A. FIG. 28D shows Loewe excess for the combination in FIG. 28A and FIG. 28E shows Bliss excess for the combination in FIG. 28A.

FIG. 29A-FIG. 29F show discovery and characterization of the novel ERK1/2 inhibitor BVD-523 (ulixertinib). FIG. 29A shows that BVD-523 demonstrates inhibition in a reversible ATP-competitive manner. This is demonstrated by a linear increase in IC₅₀ values for inhibition of ERK2 with increasing ATP concentration as shown in FIG. 29B. FIG. 29C shows a representative plot of the dose-response curve and FIG. 29D shows a plot of IC₅₀ over time. FIG. 29E shows BVD-523 binding to ERK2 and phospho-ERK2 (pERK2), compared with negative control protein p38. FIG. 29F shows BVD-523 binding to ERK2 compared with the ERK inhibitors SCH772984 and pyrazolylpyrrole.

FIG. 30A-FIG. 30D show that BVD 523 inhibits cellular proliferation and enhances caspase 3 and caspase 7 activity in vitro. FIG. 30A shows that BVD-523 demonstrates preferential activity in cells with MAPK pathway mutations, as defined by the presence of mutations in RAS family members and RAF. In addition, as shown in FIG. 30B, BVD-523 blocks sensitive cell lines in the G1 phase of the cell cycle. FIG. 30C shows that BVD-523 induced a concentration- and time-dependent increase in caspase activity in the A375, WM266, and LS411N cancer cell lines after 72 hours of exposure. FIG. 30D shows that the MAPK pathway and effector proteins are modulated by acute (4-hour) and prolonged (24-hour) BVD-523 treatment in BRAF^{V600E}-mutant A375 cells.

FIG. 31A-FIG. 31C show in vivo BVD-523 anti-tumor activity. BVD-523 monotherapy inhibits tumor growth in (FIG. 31A) A375 and (FIG. 31B) Colo205 cell line xenograft models (^{a}P<0.0001, compared with vehicle control; CPT-11 dosed on Day 14 and Day 18 only). Abbreviations: BID, twice daily; CMC, carboxymethylcellulose; QD, every day; Q4D, every 4 days. FIG. 31C shows that in Colo205 xenografts, increased ERK1/2 phosphorylation correlates with BVD-523 concentration.

FIG. 32A shows signaling effects of ERK1/2 inhibitors. Using RPPA, effects on proteins are measured in cell lines (A375, AN3Ca, Colo205, HCT116, HT29 and MIAPaca2) following treatment with ERK1/2 inhibitors BVD-523 (BVD), Vx11e (Vx), GDC-0994 (GDC), or SCH722984 (SCH). FIG. 32B shows that the ERK inhibitors BVD-523, GDC-0994, and Vx11e have differential effects on phospho-ERK (ERK 1/2 T202 Y204) compared with SCH722984; phospho-RSK (p90 RSK 380) and Cyclin D1 are inhibited by the ERK inhibitors tested. Abbreviations: BRAFi, BRAF inhibitors; MEKi, MEK inhibitors. FIG. 32C shows a western blot assay of cellular and nuclear fractions from a RKO cell line following treatment with BVD-523, trametinib, SCH722984, or dabrafenib. Histone H3 (nuclear localized protein) and HSP90 (cytoplasmically localized protein) were included as positive controls to confirm that the nuclear and cytoplasmic fractions were properly enriched; nuclear fractions have high H3 and cytoplasmic fractions have higher HSP90.

FIG. 33 shows that the ERK inhibitors BVD-523, Vx11, GDC-0994, and SCH772984 (SCH) demonstrate cell line-dependent changes in phospho-ATK levels. Abbreviation: DMSO, dimethyl sulfoxide.

FIG. 34A-FIG. 34D show that BVD-523 demonstrates activity in models of resistance to BRAF/MEK inhibition. The appearance of resistance to BVD-523, dabrafenib, or trametinib in BRAFV600E A375 cells following exposure to increasing concentrations of drug is indicated. A strict set of “criteria” was applied to determine when the dose could be increased in order to ensure that the kinetics of the acquisition of resistance between treatments was comparable. See, Example 1. Time is shown against multipliers of IC₅₀; each point on the plotted line represents a change of medium or cell split. FIG. 34A shows that adapting cells to growth in the presence of BVD-523 was more challenging than with either dabrafenib or trametinib. FIG. 34B shows that BVD-523 sensitivity is retained in A375 cells cultured to acquire resistance to combined BRAF (dabrafenib)+MEK (trametinib) inhibition. In FIG. 34C, cells were treated with compound for 96 h and viability was assessed using CellTiter-Glo®. BVD-523 activity is retained in BRAFV600E RKO cells cross-resistant to BRAF (dabrafenib) and MEK (trametinib) inhibitors due to endogenous heterozygous knock-in of MEK1^{Q56P}. FIG. 34D shows that BVD-523 inhibition of pRSK in BRAF^{V600E}-mutant cell line RKO is maintained in the presence of MEK1^{Q56P}, which confers resistance to MEK and BRAF inhibition. Knock-in of KRAS mutant alleles into SW48 cell lines significantly diminishes sensitivity to the MEK inhibitors trametinib and selumetinib, while comparatively sensitivity to BVD-523 is retained.

FIG. 35A shows BVD-523 in vivo activity in xenografts derived from a vemurafenib-relapsed patient. Mean tumor volume (±SEM) is shown for BVD-523 100 mg/kg BID alone, dabrafenib 50 mg/kg BID alone, and BVD-523 100 mg/kg BID plus dabrafenib 50 mg/kg BID. Abbreviations: BID, twice daily; SEM, standard error of mean.

FIG. 36A-FIG. 36D show the benefit of combined BVD-523 and BRAF inhibition. FIG. 36A-FIG. 36B show that the combination of BVD-523 plus dabrafenib exhibited superior antitumor activity compared with treatment with either agent alone in a A375 BRAF^{V600E}-mutant melanoma cell line xenograft model with a tumor start volume of 75-144 mm³. FIG. 36C-FIG. 36D show similar data from the same model with an enlarged tumor volume (700-800 mm³) at the start of dosing. Plots of mean tumor growth (left panels) and Kaplan-Meier survival (right panels) are presented for each study. Abbreviations: BID, twice daily; QD, once daily.

FIG. 37A shows that, in SW48 colorectal cells engineered with KRAS alleles, response to paclitaxel was unaltered compared to control. FIG. 37B shows combination interactions between BVD-523 and vemurafenib, which were assessed using an 8×10 matrix of concentrations using the Loewe Additivity and Bliss Independence Models, and analyzed with Horizon's Chalice, Bioinformatics Software. Chalice enables potential synergistic interactions to be identified by displaying the calculated excess inhibition over that predicted as being additive across the dose matrix as a heat map, and by reporting a quantitative “Synergy Score” based on the Loewe model. The results suggest that interactions between BVD-523 and vemurafenib are at least additive, and in some cases synergistic in melanoma cell lines carrying a BRAFV600E mutation. FIG. 37C shows that BVD-523 in combination with dabrafenib markedly delays the onset of acquired resistance in A375 BRAFV600E melanoma cells. The temporal acquisition of resistance in response to escalating concentrations of dabrafenib alone or in combination with BVD-523 or trametinib was assessed. Strict criteria were applied as to when the dose could be increased to ensure that the kinetics of adaptation was comparable between treatments. See, Example 1.

FIG. 38 shows that BVD-523 inhibits ex vivo PMA-stimulated RSK1/2 phosphorylation in human whole blood. Averages of BVD-523 concentration data set are indicated by (-). n=20 for each concentration of BVD-523. Abbreviations: PBMC, peripheral blood mononuclear cells; RSK, ribosomal S6 kinase.

FIG. 39A shows steady-state BVD-523 pharmacokinetics (Cycle 1, Day 15). The dashed red line indicates an EC₅₀ 200 ng/mL HWB. Abbreviations: AUC, area under the curve; BID, twice daily; Cₘₐₓ, maximum concentration; EC₅₀, 50% maximum effective concentration; HWB, human whole blood; SD, standard deviation. FIG. 39B shows pharmacodynamic inhibition of RSK phosphorylation by BVD-523 in human whole blood. Abbreviations: BID, twice daily; pRSK, phospho-RSK; RSK, ribosomal S6 kinase.

FIG. 40A shows the best radiographic response in patients treated with BVD-523. Included are all patients with disease measured by RECIST v1.1 who received ≥1 dose of study treatment and had >1 on-treatment tumor assessment (25/27; 2 did not receive both scans of target lesions). Response was measured as the change from baseline in the sum of the longest diameter of each target lesion. Dose shown is that which the patient was receiving at the time of response. The dashed line indicates the threshold for a partial response according to RECIST v1.1. Abbreviations: CRC, colorectal cancer; NET, neuroendocrine tumors; NSCLC, non-small cell lung cancer; NSGCT, nonseminomatous germ cell tumors; PNET, pancreatic NET; PTC, papillary thyroid cancer; RECIST v1.1, Response Evaluation Criteria in Solid Tumors version 1.1; SLD, sum of the largest diameter. FIG. 40B shows a computerized tomography scan of a confirmed partial response in a 61-year-old patient with a BRAF-mutant melanoma treated with BVD-523.

FIG. 41 shows tumor response and tumor progression. Shown is a swimmer plot of tumor response, tumor progression, and duration of treatment in response-evaluable patients treated with BVD-523. Origin of the vertical axis corresponds to randomization date or reference start date. Analysis cut-off date: Dec. 1, 2015. Abbreviation: BID, twice daily.

### DETAILED DESCRIPTION OF THE INVENTION

One embodiment of the present invention is a method for treating or ameliorating the effects of a cancer in a subject, which cancer is refractory or resistant to non-ERK MAPK pathway inhibitor therapy. The method comprises administering to the subject an effective amount of BVD-523 or a pharmaceutically acceptable salt thereof.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present invention include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc.

In the present invention, BVD-523 corresponds to a compound according to formula (I):

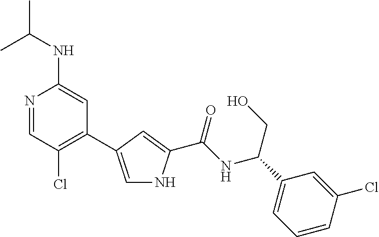 [SMILES: CC(C)Nc1cc(-c2c[nH]c(C(=O)N[C@H](CO)c3cccc(Cl)c3)c2)c(Cl)cn1]

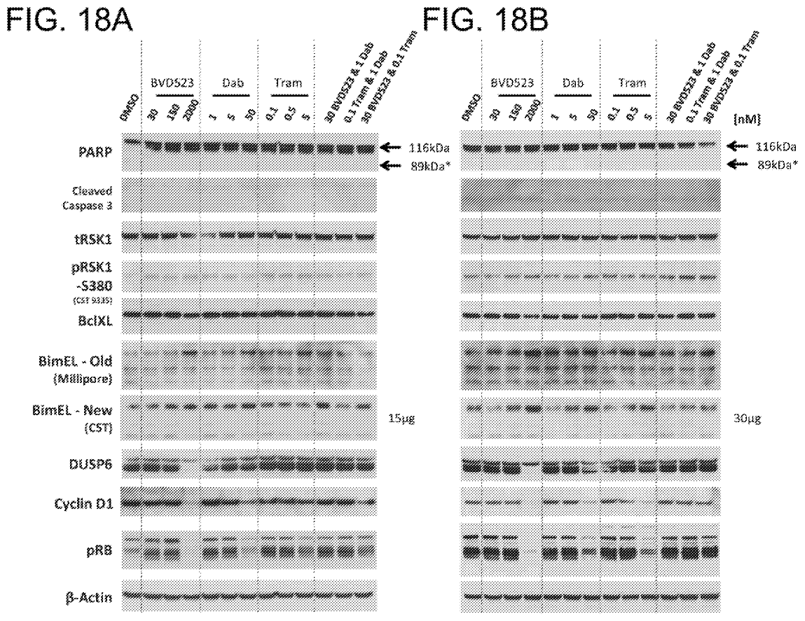

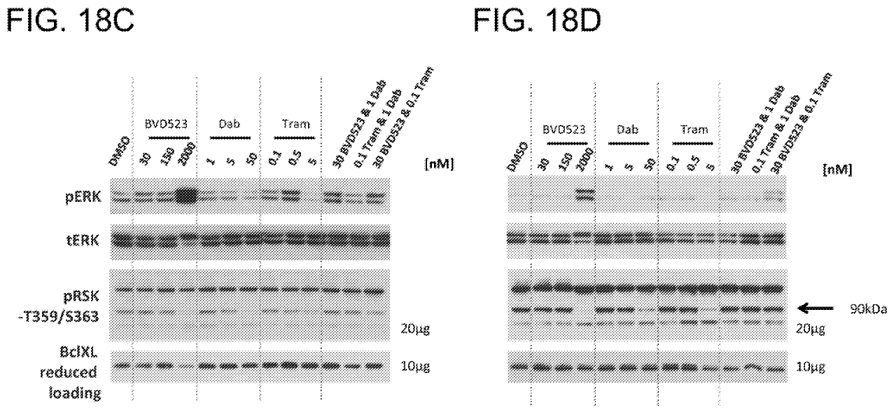

and pharmaceutically acceptable salts thereof. BVD-523 may be synthesized according to the methods disclosed, e.g., in U.S. Pat. No. 7,354,939. Enantiomers and racemic mixtures of both enantiomers of BVD-523 are also contemplated within the scope of the present invention. BVD-523 is an ERK1/2 inhibitor with a mechanism of action that is believed to be, e.g., unique and distinct from certain other ERK1/2 inhibitors, such as SCH772984 and the pyrimidinal structure used by Hatzivassiliou et al. (2012). For example, other ERK1/2 inhibitors, such as SCH772984, inhibit autophosphorylation of ERK (Morris et al., 2013), whereas BVD-523 allows for the autophosphorylation of ERK while still inhibiting ERK. (See, e.g., FIG. 18).

As used herein, the words “resistant” and “refractory” are used interchangeably. Being “resistant” to non-ERK MAPK pathway inhibitor therapy treatments means that non-ERK MAPK inhibitors have reduced efficacy in treating cancer.

As used herein, a “non-ERK MAPK inhibitor” means any substance that reduces the activity, expression or phosphorylation of proteins or other members of the MAPK pathway that results in a reduction of cell growth or an increase in cell death, with the exception of ERK1/2 inhibitors. As used herein, an “ERK1/2 inhibitor” means those substances that (i) directly interact with ERK1 and/or ERK2, e.g., by binding to ERK1/2 and (ii) decrease the expression or the activity of ERK1 and/or ERK2 protein kinases. Therefore, inhibitors that act upstream of ERK1/2, such as MEK inhibitors and RAF inhibitors, are not ERK1/2 inhibitors according to the present invention (but they are non-ERK MAPK inhibitors). Non-limiting examples of ERK1/2 inhibitors according to the present invention include AEZS-131 (Aeterna Zentaris), AEZS-136 (Aeterna Zentaris), BVD-523 (BioMed Valley Discoveries, Inc.), SCH-722984 (Merck & Co.), SCH-772984 (Merck & Co.), SCH-900353 (MK-8353) (Merck & Co.), pharmaceutically acceptable salts thereof, and combinations thereof.

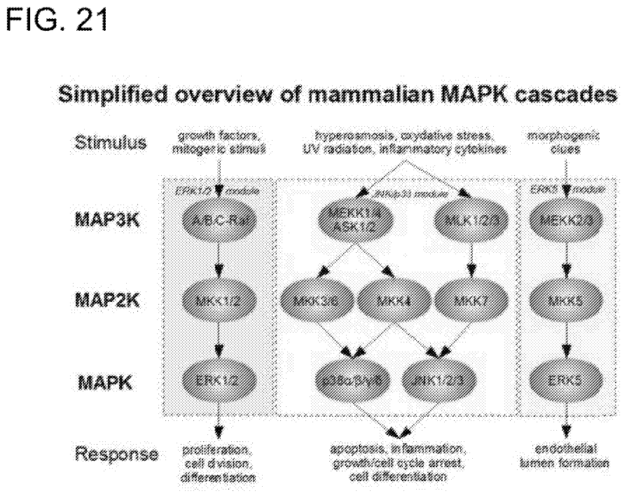

An overview of the mammalian MAPK cascades is shown in FIG. 21. The MAPK pathway is reviewed in e.g., Akinleye et al., 2013. Briefly, with respect to the ERK1/2 module in FIG. 21 (light purple box), the MAPK 1/2 signaling cascade is activated by ligand binding to receptor tyrosine kinases (RTK). The activated receptors recruit and phosphorylate adaptor proteins Grb2 and SOS, which then interact with membrane-bound GTPase Ras and cause its activation. In its activated GTP-bound form, Ras recruits and activates RAF kinases (A-RAF, B-RAF, and C-RAF/RAF-1). The activated RAF kinases activate MAPK 1/2 (MKK1/2), which in turn catalyzes the phosphorylation of threonine and tyrosine residues in the activation sequence Thr-Glu-Tyr of ERK1/2. With respect to the JNK/p38 module (yellow box in FIG. 21), upstream kinases, MAP3Ks, such as MEKK1/4, ASK1/2, and MLK1/2/3, activate MAP2K3/6 (MKK3/6), MAP2K4 (MKK4), and MAP2K7 (MKK7). These MAP2K's then activate JNK protein kinases, including JNK1, JNK2, and JNK3, as well as p38 α/β/γ/δ. To execute their functions, JNKs activate several transcription factors, including c-Jun, ATF-2, NF-ATc1, HSF-1 and STAT3. With respect to the ERK5 module (blue box in FIG. 21), the kinases upstream of MAP2K5 (MKK5) are MEKK2 and MEKK3. The best characterized downstream target of MEK5 is ERK5, also known as big MAP kinase 1 (BMK1) because it is twice the size of other MAPKs.

Non-limiting examples of non-ERK MAPK pathway inhibitors according to the present invention include RAS inhibitors, RAF inhibitors (such as, e.g., inhibitors of A-RAF, B-RAF, C-RAF (RAF-1)), MEK inhibitors, and combinations thereof. Preferably, the non-ERK MAPK pathway inhibitors are BRAF inhibitors, MEK inhibitors, and combinations thereof.

As used herein, a “RAS inhibitor” means those substances that (i) directly interact with RAS, e.g., by binding to RAS and (ii) decrease the expression or the activity of RAS. Non-limiting exemplary RAS inhibitors include, but are not limited to, farnesyl transferase inhibitors (such as, e.g., tipifarnib and lonafarnib), farnesyl group-containing small molecules (such as, e.g., salirasib and TLN-4601), DCAI, as disclosed by Maurer (Maurer et al., 2012), Kobe0065 and and Kobe2602, as disclosed by Shima (Shima et al., 2013), HBS 3 (Patgiri et al., 2011), and AIK-4 (Allinky).

As used herein, a “RAF inhibitor” means those substances that (i) directly interact with RAF, e.g., by binding to RAF and (ii) decrease the expression or the activity of RAF, such as, e.g., A-RAF, B-RAF, and C-RAF (RAF-1). Non-limiting exemplary RAF inhibitors, including BRAF inhibitors, include:

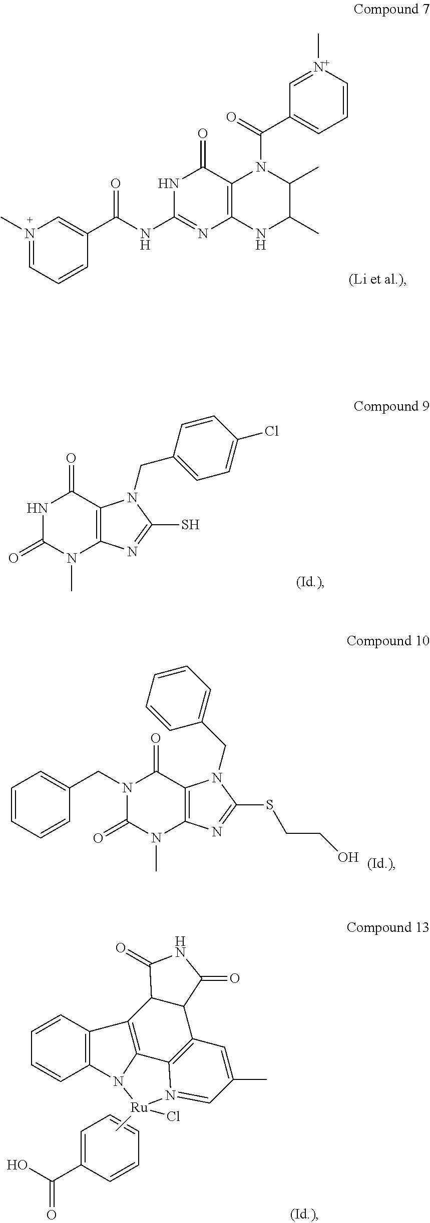

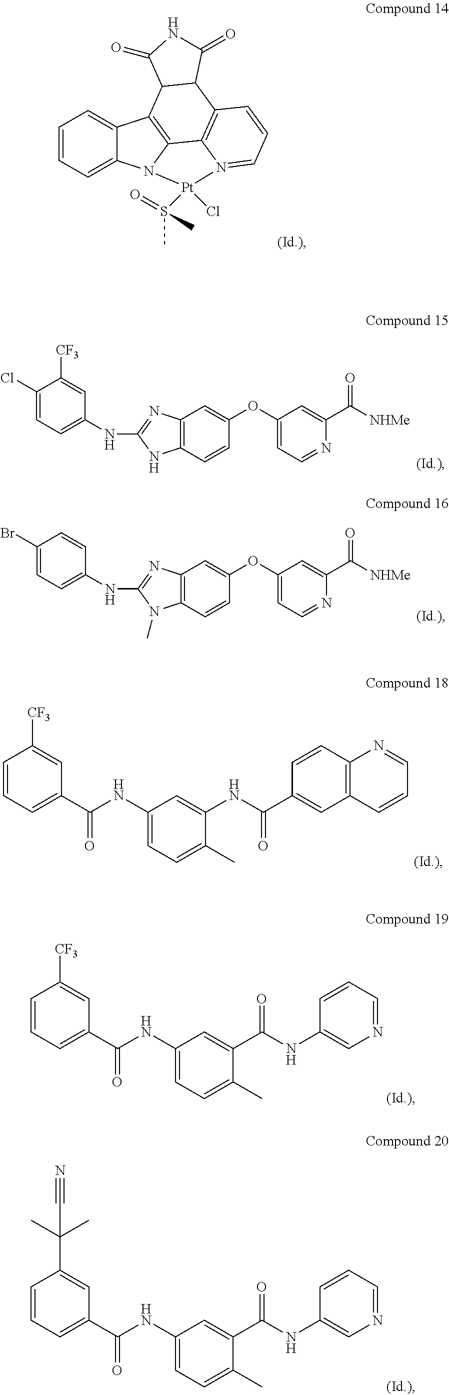

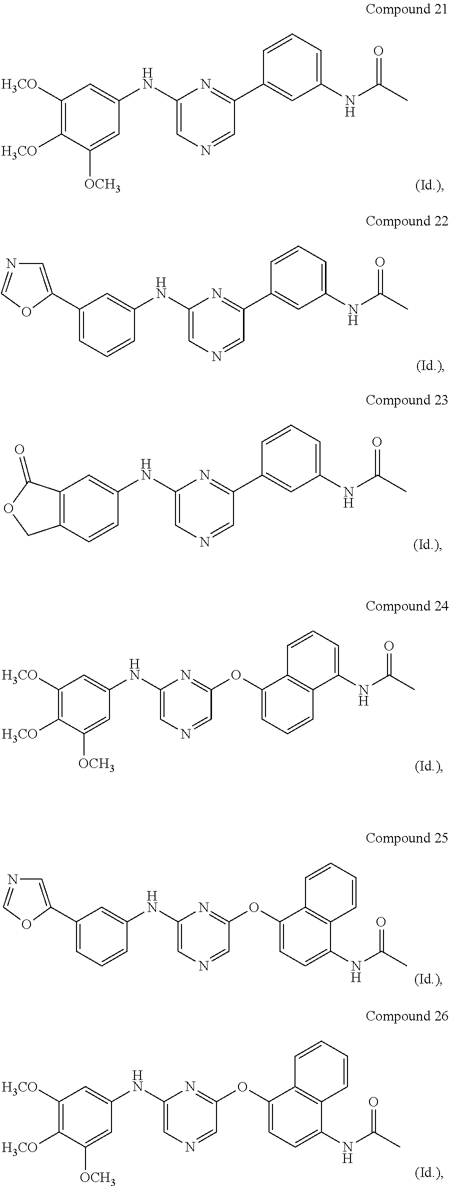 [SMILES: COc1cc(Nc2cncc(-c3cccc(NC(C)=O)c3)n2)cc(OC)c1OC ; CC(=O)Nc1cccc(-c2cncc(Nc3cccc(-c4cnco4)c3)n2)c1 ; CC(=O)Nc1cccc(-c2cncc(Nc3ccc4c(c3)C(=O)OC4)n2)c1 ; COc1cc(Nc2cncc(Oc3cccc4c(NC(C)=O)cccc34)n2)cc(OC)c1OC ; CC(=O)Nc1ccc(Oc2cncc(Nc3cccc(-c4cnco4)c3)n2)c2ccccc12 ; COc1cc(Nc2cncc(Oc3ccc(NC(C)=O)c4ccccc34)n2)cc(OC)c1OC]

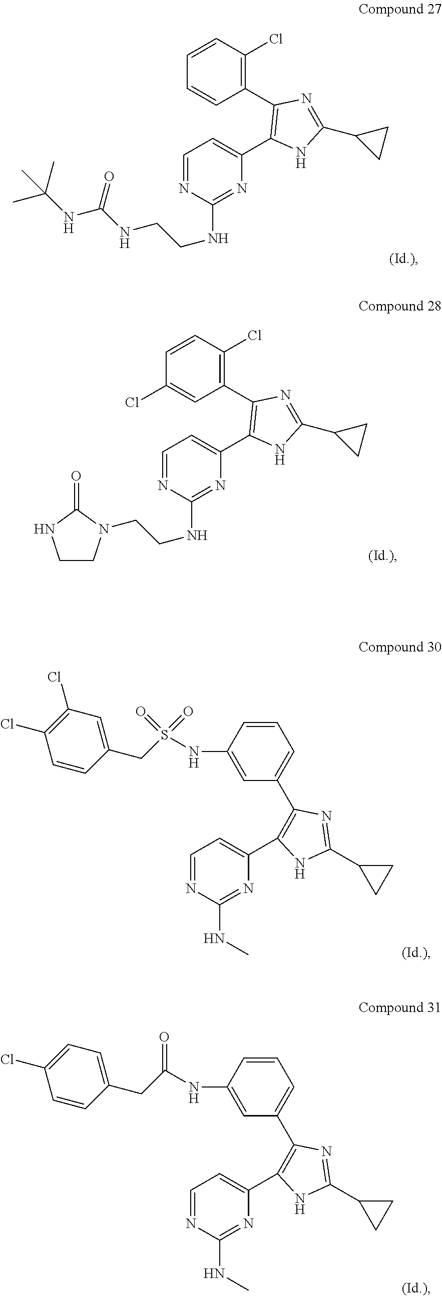 [SMILES: CC(C)(C)NC(=O)NCCNc1nccc(-c2[nH]c(C3CC3)nc2-c2ccccc2Cl)n1 ; O=C1NCCN1CCNc1nccc(-c2[nH]c(C3CC3)nc2-c2cc(Cl)ccc2Cl)n1 ; CNc1nccc(-c2[nH]c(C3CC3)nc2-c2cccc(NS(=O)(=O)Cc3ccc(Cl)c(Cl)c3)c2)n1 ; CNc1nccc(-c2[nH]c(C3CC3)nc2-c2cccc(NC(=O)Cc3ccc(Cl)cc3)c2)n1]

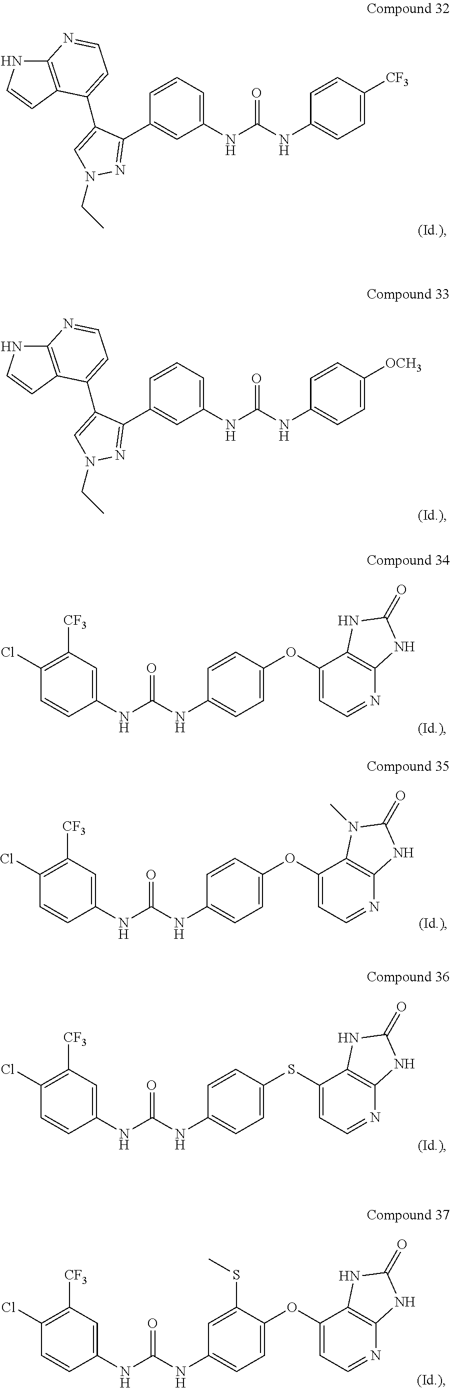 [SMILES: CCn1cc(-c2ccnc3[nH]ccc23)c(-c2cccc(NC(=O)Nc3ccc(C(F)(F)F)cc3)c2)n1 ; CCn1cc(-c2ccnc3[nH]ccc23)c(-c2cccc(NC(=O)Nc3ccc(OC)cc3)c2)n1 ; O=C(Nc1ccc(Oc2ccnc3[nH]c(=O)[nH]c23)cc1)Nc1ccc(Cl)c(C(F)(F)F)c1 ; Cn1c(=O)[nH]c2nccc(Oc3ccc(NC(=O)Nc4ccc(Cl)c(C(F)(F)F)c4)cc3)c21 ; O=C(Nc1ccc(Sc2ccnc3[nH]c(=O)[nH]c23)cc1)Nc1ccc(Cl)c(C(F)(F)F)c1 ; CSc1cc(NC(=O)Nc2ccc(Cl)c(C(F)(F)F)c2)ccc1Oc1ccnc2[nH]c(=O)[nH]c12]

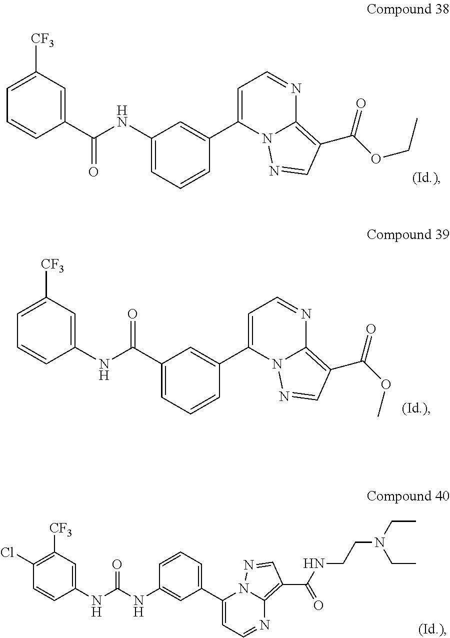 [SMILES: CCOC(=O)c1cnn2c(-c3cccc(NC(=O)c4cccc(C(F)(F)F)c4)c3)ccnc12 ; COC(=O)c1cnn2c(-c3cccc(C(=O)Nc4cccc(C(F)(F)F)c4)c3)ccnc12 ; CCN(CC)CCNC(=O)c1cnn2c(-c3cccc(NC(=O)Nc4ccc(Cl)c(C(F)(F)F)c4)c3)ccnc12]

AAL881 (Novartis); AB-024 (Ambit Biosciences), ARQ-736 (ArQule), ARQ-761 (ArQule), AZ628 (Axon Medchem BV), BeiGene-283 (BeiGene), BIIB-024 (MLN 2480) (Sunesis & Takeda), b-raf inhibitor (Sareum), BRAF kinase inhibitor (Selexagen Therapeutics), BRAF siRNA 313 (tacaccagcaagctagatgca) and 523 (cctatcgttagagtcttcctg) (Liu et al., 2007), CTT239065 (Institute of Cancer Research), dabrafenib (GSK2118436), DP-4978 (Deciphera Pharmaceuticals), HM-95573 (Hanmi), GDC-0879 (Genentech), GW-5074 (Sigma Aldrich), ISIS 5132 (Novartis), L779450 (Merck), LBT613 (Novartis), LErafAON (NeoPharm, Inc.), LGX-818 (Novartis), pazopanib (GlaxoSmithKline), PLX3202 (Plexxikon), PLX4720 (Plexxikon), PLX5568 (Plexxikon), RAF-265 (Novartis), RAF-365 (Novartis), regorafenib (Bayer Healthcare Pharmaceuticals, Inc.), RO 5126766 (Hoffmann-La Roche), SB-590885 (GlaxoSmithKline), SB699393 (GlaxoSmithKline), sorafenib (Onyx Pharmaceuticals), TAK 632 (Takeda), TL-241 (Teligene), vemurafenib (RG7204 or PLX4032) (Daiichi Sankyo), XL-281 (Exelixis), ZM-336372 (AstraZeneca), pharmaceutically acceptable salts thereof, and combinations thereof.

As used herein, a “MEK inhibitor” means those substances that (i) directly interact with MEK, e.g., by binding to MEK and (ii) decrease the expression or the activity of MEK. Thus, inhibitors that act upstream of MEK, such as RAS inhibitors and RAF inhibitors, are not MEF inhibitors according to the present invention. Non-limiting examples of MEK inhibitors include anthrax toxin, antroquinonol (Golden Biotechnology), ARRY-142886 (6-(4-bromo-2-chloro-phenylamino)-7-fluoro-3-methyl-3H-benzoimidazole-5-carboxylic acid (2-hydroxy-ethoxy)-amide) (Array BioPharma), ARRY-438162 (Array BioPharma), AS-1940477 (Astellas), AS-703988 (Merck KGaA), bentamapimod (Merck KGaA), BI-847325 (Boehringer Ingelheim), E-6201 (Eisai), GDC-0623 (Hoffmann-La Roche), GDC-0973 (cobimetinib) (Hoffmann-La Roche), L783277 (Merck), lethal factor portion of anthrax toxin, MEK162 (Array BioPharma), PD 098059 (2-(2′-amino-3′-methoxyphenyl)-oxanaphthalen-4-one) (Pfizer), PD 184352 (CI-1040) (Pfizer), PD-0325901 (Pfizer), pimasertib (Santhera Pharmaceuticals), RDEA119 (Ardea Biosciences/Bayer), refametinib (AstraZeneca), RG422 (Chugai Pharmaceutical Co.), RO092210 (Roche), RO4987655 (Hoffmann-La Roche), RO5126766 (Hoffmann-La Roche), selumetinib (AZD6244) (AstraZeneca), SL327 (Sigma), TAK-733 (Takeda), trametinib (Japan Tobacco), U0126 (1,4-diamino-2,3-dicyano-1,4-bis(2-aminophenylthio) butadiene) (Sigma), WX-554 (Wilex), YopJ polypeptide (Mittal et al., 2010), pharmaceutically acceptable salts thereof, and combinations thereof.

In one aspect of this embodiment, substantially all phosphorylation of ribosomal s6 kinase (RSK) is inhibited after administration of BVD-523 or a pharmaceutically acceptable salt thereof. As used herein in the context of RSK phosphorylation, “substantially all” means a reduction of greater than 50% reduction, preferably greater than 55%, 60%, 65%, 70%, 75%, 80%, 85%, 90%, 91%, 92%, 93%, 94%, 95%, 96%, 97%, 98%, or 99% reduction.

In another aspect of this embodiment, the cancer has MAPK activity. As used herein, having “MAPK activity” means that proteins downstream of ERK are still active, even if proteins upstream of ERK may not be active. Such a cancer may be a solid tumor cancer or a hematologic cancer.

In the present invention, cancers include both solid and hematologic cancers. Non-limiting examples of solid cancers include adrenocortical carcinoma, anal cancer, bladder cancer, bone cancer (such as osteosarcoma), brain cancer, breast cancer, carcinoid cancer, carcinoma, cervical cancer, colon cancer, endometrial cancer, esophageal cancer, extrahepatic bile duct cancer, Ewing family of cancers, extracranial germ cell cancer, eye cancer, gallbladder cancer, gastric cancer, germ cell tumor, gestational trophoblastic tumor, head and neck cancer, hypopharyngeal cancer, islet cell carcinoma, kidney cancer, large intestine cancer, laryngeal cancer, leukemia, lip and oral cavity cancer, liver tumor/cancer, lung tumor/cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell cancer, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sezary syndrome, skin cancers (such as cutaneous t-cell lymphoma, Kaposi's sarcoma, mast cell tumor, and melanoma), small intestine cancer, soft tissue sarcoma, stomach cancer, testicular cancer, thymoma, thyroid cancer, urethral cancer, uterine cancer, vaginal cancer, vulvar cancer, and Wilms' tumor.

Examples of hematologic cancers include, but are not limited to, leukemias, such as adult/childhood acute lymphoblastic leukemia, adult/childhood acute myeloid leukemia, chronic lymphocytic leukemia, chronic myelogenous leukemia, and hairy cell leukemia, lymphomas, such as AIDS-related lymphoma, cutaneous T-cell lymphoma, adult/childhood Hodgkin lymphoma, mycosis fungoides, adult/childhood non-Hodgkin lymphoma, primary central nervous system lymphoma, Sézary syndrome, cutaneous T-cell lymphoma, and Waldenstrom macroglobulinemia, as well as other proliferative disorders such as chronic myeloproliferative disorders, Langerhans cell histiocytosis, multiple myeloma/plasma cell neoplasm, myelodysplastic syndromes, and myelodysplastic/myeloproliferative neoplasms.

Preferably, the cancer is selected from the group consisting of a cancer of the large intestine, breast cancer, pancreatic cancer, skin cancer, and endometrial cancers. More preferably, the cancer is melanoma.

In another aspect of this embodiment, the method further comprises administering to the subject at least one additional therapeutic agent effective for treating or ameliorating the effects of the cancer. The additional therapeutic agent may be selected from the group consisting of an antibody or fragment thereof, a cytotoxic agent, a toxin, a radionuclide, an immunomodulator, a photoactive therapeutic agent, a radiosensitizing agent, a hormone, an anti-angiogenesis agent, and combinations thereof.

As used herein, an “antibody” encompasses naturally occurring immunoglobulins as well as non-naturally occurring immunoglobulins, including, for example, single chain antibodies, chimeric antibodies (e.g., humanized murine antibodies), and heteroconjugate antibodies (e.g., bispecific antibodies). Fragments of antibodies include those that bind antigen, (e.g., Fab′, F(ab′)₂, Fab, Fv, and rIgG). See also, e.g., Pierce Catalog and Handbook, 1994-1995 (Pierce Chemical Co., Rockford, III.); Kuby, J., Immunology, 3rd Ed., W.H. Freeman & Co., New York (1998). The term antibody also includes bivalent or bispecific molecules, diabodies, triabodies, and tetrabodies. The term “antibody” further includes both polyclonal and monoclonal antibodies.

Examples of therapeutic antibodies that may be used in the present invention include rituximab (Rituxan), Cetuximab (Erbitux), bevacizumab (Avastin), and Ibritumomab (Zevalin).

Cytotoxic agents according to the present invention include DNA damaging agents, antimetabolites, anti-microtubule agents, antibiotic agents, etc. DNA damaging agents include alkylating agents, platinum-based agents, intercalating agents, and inhibitors of DNA replication. Non-limiting examples of DNA alkylating agents include cyclophosphamide, mechlorethamine, uramustine, melphalan, chlorambucil, ifosfamide, carmustine, lomustine, streptozocin, busulfan, temozolomide, pharmaceutically acceptable salts thereof, prodrugs, and combinations thereof. Non-limiting examples of platinum-based agents include cisplatin, carboplatin, oxaliplatin, nedaplatin, satraplatin, triplatin tetranitrate, pharmaceutically acceptable salts thereof, prodrugs, and combinations thereof. Non-limiting examples of intercalating agents include doxorubicin, daunorubicin, idarubicin, mitoxantrone, pharmaceutically acceptable salts thereof, prodrugs, and combinations thereof. Non-limiting examples of inhibitors of DNA replication include irinotecan, topotecan, amsacrine, etoposide, etoposide phosphate, teniposide, pharmaceutically acceptable salts thereof, prodrugs, and combinations thereof. Antimetabolites include folate antagonists such as methotrexate and premetrexed, purine antagonists such as 6-mercaptopurine, dacarbazine, and fludarabine, and pyrimidine antagonists such as 5-fluorouracil, arabinosylcytosine, capecitabine, gemcitabine, decitabine, pharmaceutically acceptable salts thereof, prodrugs, and combinations thereof. Anti-microtubule agents include without limitation vinca alkaloids, paclitaxel (Taxol®), docetaxel (Taxotere®), and ixabepilone (Ixempra®). Antibiotic agents include without limitation actinomycin, anthracyclines, valrubicin, epirubicin, bleomycin, plicamycin, mitomycin, pharmaceutically acceptable salts thereof, prodrugs, and combinations thereof.

Cytotoxic agents according to the present invention also include an inhibitor of the PI3K/Akt pathway. Non-limiting examples of an inhibitor of the PI3K/Akt pathway include A-674563 (CAS #552325-73-2), AGL 2263, AMG-319 (Amgen, Thousand Oaks, CA), AS-041164 (5-benzo[1,3]dioxol-5-ylmethylene-thiazolidine-2,4-dione), AS-604850 (5-(2,2-Difluoro-benzo[1,3]dioxol-5-ylmethylene)-thiazolidine-2,4-dione), AS-605240 (5-quinoxilin-6-methylene-1,3-thiazolidine-2,4-dione), AT7867 (CAS #857531-00-1), benzimidazole series, Genentech (Roche Holdings Inc., South San Francisco, CA), BML-257 (CAS #32387-96-5), CAL-120 (Gilead Sciences, Foster City, CA), CAL-129 (Gilead Sciences), CAL-130 (Gilead Sciences), CAL-253 (Gilead Sciences), CAL-263 (Gilead Sciences), CAS #612847-09-3, CAS #681281-88-9, CAS #75747-14-7, CAS #925681-41-0, CAS #98510-80-6, CCT128930 (CAS #885499-61-6), CH5132799 (CAS #1007207-67-1), CHR-4432 (Chroma Therapeutics, Ltd., Abingdon, UK), FPA 124 (CAS #902779-59-3), GS-1101 (CAL-101) (Gilead Sciences), GSK 690693 (CAS #937174-76-0), H-89 (CAS #127243-85-0), Honokiol, IC87114 (Gilead Science), IPI-145 (Intellikine Inc.), KAR-4139 (Karus Therapeutics, Chilworth, UK), KAR-4141 (Karus Therapeutics), KIN-1 (Karus Therapeutics), KT 5720 (CAS #108068-98-0), Miltefosine, MK-2206 dihydrochloride (CAS #1032350-13-2), ML-9 (CAS #105637-50-1), Naltrindole Hydrochloride, OXY-111A (NormOxys Inc., Brighton, MA), perifosine, PHT-427 (CAS #1191951-57-1), PI3 kinase delta inhibitor, Merck KGaA (Merck & Co., Whitehouse Station, NJ), PI3 kinase delta inhibitors, Genentech (Roche Holdings Inc.), PI3 kinase delta inhibitors, Incozen (Incozen Therapeutics, Pvt. Ltd., Hydrabad, India), PI3 kinase delta inhibitors-2, Incozen (Incozen Therapeutics), PI3 kinase inhibitor, Roche-4 (Roche Holdings Inc.), PI3 kinase inhibitors, Roche (Roche Holdings Inc.), PI3 kinase inhibitors, Roche-5 (Roche Holdings Inc.), PI3-alpha/delta inhibitors, Pathway Therapeutics (Pathway Therapeutics Ltd., South San Francisco, CA), PI3-delta inhibitors, Cellzome (Cellzome AG, Heidelberg, Germany), PI3-delta inhibitors, Intellikine (Intellikine Inc., La Jolla, CA), PI3-delta inhibitors, Pathway Therapeutics-1 (Pathway Therapeutics Ltd.), PI3-delta inhibitors, Pathway Therapeutics-2 (Pathway Therapeutics Ltd.), PI3-delta/gamma inhibitors, Cellzome (Cellzome AG), PI3-delta/gamma inhibitors, Cellzome (Cellzome AG), PI3-delta/gamma inhibitors, Intellikine (Intellikine Inc.), PI3-delta/gamma inhibitors, Intellikine (Intellikine Inc.), PI3-delta/gamma inhibitors, Pathway Therapeutics (Pathway Therapeutics Ltd.), PI3-delta/gamma inhibitors, Pathway Therapeutics (Pathway Therapeutics Ltd.), PI3-gamma inhibitor Evotec (Evotec), PI3-gamma inhibitor, Cellzome (Cellzome AG), PI3-gamma inhibitors, Pathway Therapeutics (Pathway Therapeutics Ltd.), PI3K delta/gamma inhibitors, Intellikine-1 (Intellikine Inc.), PI3K delta/gamma inhibitors, Intellikine-1 (Intellikine Inc.), pictilisib (Roche Holdings Inc.), PIK-90 (CAS #677338-12-4), SC-103980 (Pfizer, New York, NY), SF-1126 (Semafore Pharmaceuticals, Indianapolis, IN), SH-5, SH-6, Tetrahydro Curcumin, TG100-115 (Targegen Inc., San Diego, CA), Triciribine, X-339 (Xcovery, West Palm Beach, FL), XL-499 (Evotech, Hamburg, Germany), pharmaceutically acceptable salts thereof, and combinations thereof.

In the present invention, the term “toxin” means an antigenic poison or venom of plant or animal origin. An example is diphtheria toxin or portions thereof.

In the present invention, the term “radionuclide” means a radioactive substance administered to the patient, e.g., intravenously or orally, after which it penetrates via the patient's normal metabolism into the target organ or tissue, where it delivers local radiation for a short time. Examples of radionuclides include, but are not limited to, I-125, At-211, Lu-177, Cu-67, I-131, Sm-153, Re-186, P-32, Re-188, In-114m, and Y-90.

In the present invention, the term “immunomodulator” means a substance that alters the immune response by augmenting or reducing the ability of the immune system to produce antibodies or sensitized cells that recognize and react with the antigen that initiated their production. Immunomodulators may be recombinant, synthetic, or natural preparations and include cytokines, corticosteroids, cytotoxic agents, thymosin, and immunoglobulins. Some immunomodulators are naturally present in the body, and certain of these are available in pharmacologic preparations. Examples of immunomodulators include, but are not limited to, granulocyte colony-stimulating factor (G-CSF), interferons, imiquimod and cellular membrane fractions from bacteria, IL-2, IL-7, IL-12, CCL3, CCL26, CXCL7, and synthetic cytosine phosphate-guanosine (CpG).

In the present invention, the term “photoactive therapeutic agent” means compounds and compositions that become active upon exposure to light. Certain examples of photoactive therapeutic agents are disclosed, e.g., in U.S. Patent Application Ser. No. 2011/0152230 A1, “Photoactive Metal Nitrosyls For Blood Pressure Regulation And Cancer Therapy.”

In the present invention, the term “radiosensitizing agent” means a compound that makes tumor cells more sensitive to radiation therapy. Examples of radiosensitizing agents include misonidazole, metronidazole, tirapazamine, and trans sodium crocetinate.

In the present invention, the term “hormone” means a substance released by cells in one part of a body that affects cells in another part of the body. Examples of hormones include, but are not limited to, prostaglandins, leukotrienes, prostacyclin, thromboxane, amylin, antimullerian hormone, adiponectin, adrenocorticotropic hormone, angiotensinogen, angiotensin, vasopressin, atriopeptin, brain natriuretic peptide, calcitonin, cholecystokinin, corticotropin-releasing hormone, encephalin, endothelin, erythropoietin, follicle-stimulating hormone, galanin, gastrin, ghrelin, glucagon, gonadotropin-releasing hormone, growth hormone-releasing hormone, human chorionic gonadotropin, human placental lactogen, growth hormone, inhibin, insulin, somatomedin, leptin, liptropin, luteinizing hormone, melanocyte stimulating hormone, motilin, orexin, oxytocin, pancreatic polypeptide, parathyroid hormone, prolactin, prolactin releasing hormone, relaxin, renin, secretin, somatostain, thrombopoietin, thyroid-stimulating hormone, testosterone, dehydroepiandrosterone, androstenedione, dihydrotestosterone, aldosterone, estradiol, estrone, estriol, cortisol, progesterone, calcitriol, and calcidiol.

Some compounds interfere with the activity of certain hormones or stop the production of certain hormones. These hormone-interfering compounds include, but are not limited to, tamoxifen (Nolvadex®), anastrozole (Arimidex®), letrozole (Femara®), and fulvestrant (Faslodex®). Such compounds are also within the meaning of hormone in the present invention.

As used herein, an “anti-angiogenesis” agent means a substance that reduces or inhibits the growth of new blood vessels, such as, e.g., an inhibitor of vascular endothelial growth factor (VEGF) and an inhibitor of endothelial cell migration. Anti-angiogenesis agents include without limitation 2-methoxyestradiol, angiostatin, bevacizumab, cartilage-derived angiogenesis inhibitory factor, endostatin, IFN-α, IL-12, itraconazole, linomide, platelet factor-4, prolactin, SU5416, suramin, tasquinimod, tecogalan, tetrathiomolybdate, thalidomide, thrombospondin, thrombospondin, TNP-470, ziv-aflibercept, pharmaceutically acceptable salts thereof, prodrugs, and combinations thereof.

Another embodiment of the present invention is a method for treating or ameliorating the effects of a cancer in a subject. The method comprises:
(a) identifying a subject with cancer that has become refractory or resistant to BRAF inhibitor therapy, MEK inhibitor therapy, or BRAF and MEK inhibitor therapy; and
(b) administering to the subject with said refractory or resistant cancer an effective amount of an ERK inhibitor, which is BVD-523 or a pharmaceutically acceptable salt thereof.

Suitable and preferred subjects are as disclosed herein. In this embodiment, the methods may be used to treat the cancers disclosed above. In accordance with the present invention, the cancer may have MAPK activity.

In one aspect of this embodiment, identifying a subject with cancer that is refractory or resistant to BRAF and/or MEK inhibitor therapy comprises:
(a) obtaining a biological sample from the subject; and
(b) screening the sample to determine whether the subject has become resistant to an inhibitor therapy selected from the group consisting of BRAF inhibitor therapy, MEK inhibitor therapy, and combinations thereof.

In the present invention, biological samples include, but are not limited to, blood, plasma, urine, skin, saliva, and biopsies. Biological samples are obtained from a subject by routine procedures and methods which are known in the art.

Preferably, screening for a cancer that is refractory or resistant to BRAF inhibitor therapy may comprise, e.g., identifying (i) a switch between RAF isoforms, (ii) upregulation of RTK or NRAS signaling, (iii) reactivation of mitogen activated protein kinase (MAPK) signaling, (iv) the presence of a MEK activating mutation, and combinations thereof.

A switch between RAF isoforms may occur in subjects having acquired resistance to BRAF inhibitor therapy. To detect such a switch, BRAF inhibitor-resistant tumor cells may be retrieved from a patient and analyzed via Western blotting for ERK and phospho-ERK levels in the presence of a BRAF inhibitor. Comparison with BRAF inhibitor-sensitive cells treated with a BRAF inhibitor may reveal higher levels of phospho-ERK in BRAF inhibitor-resistant tumor cells, implying that a switch has taken place in which another RAF isoform phosphorylates ERK in place of BRAF. Confirmation of which RAF isoform has taken over may involve sh/siRNA-mediated knockdown of ARAF and CRAF individually in BRAF inhibitor-resistant cells exposed to a BRAF inhibitor, followed by subsequent Western blotting for ERK and phospho-ERK levels. If, for example, ARAF knockdown in BRAF inhibitor-resistant cells exposed to a BRAF inhibitor still results in high levels of phospho-ERK, it would indicate that CRAF has taken over phosphorylating ERK. Likewise, if CRAF was knocked down in BRAF inhibitor-resistant cells exposed to BRAF inhibitor and ERK was still highly phosphorylated, it would mean that ARAF has taken over ERK phosphorylation. RAF isoform switching may also involve simultaneous knockdown of ARAF and CRAF in BRAF inhibitor-resistant cells in the presence of BRAF inhibitor, effectively blocking all RAF-mediated phosphorylation. A resulting decrease in ERK phosphorylation would indicate that the BRAF inhibitor-resistant cells have the capacity to switch between RAF isoforms in order to phosphorylate ERK (Villanueva, et al., 2010).

Upregulation of RTK or NRAS signaling may also be a cause of BRAF inhibitor resistance. Detection may, e.g., first involve using Western blotting protocols with phospho-specific antibodies to analyze the activation of the downstream RAF effectors MEK1/2 and ERK1/2. If BRAF inhibitor-resistant cells show high activation levels of these proteins in the presence of a BRAF inhibitor, RTK or NRAS upregulation may be the cause. Gene expression profiling (or other related methods) of BRAF inhibitor-resistant cells in the presence of a BRAF inhibitor may reveal higher expression levels of KIT, MET, EGFR, and PDGFRβ RTKs as compared to BRAF inhibitor-sensitive cells. Real-time quantitative polymerase chain reaction experiments, or other similar procedures, focusing on any of these genes may confirm higher expression levels while phospho-RTK arrays (R&D Systems, Minneapolis, MN) may show elevated activation-associated tyrosine phosphorylation. Alternatively, NRAS activation may be detected by various gene sequencing protocols. Activating mutations in NRAS, particularly Q61K, may indicate that B-RAF signaling has been bypassed. In melanoma cells, activated NRAS uses C-RAF to signal to MEK-ERK. Thus, activated NRAS may enable a similar bypass pathway in BRAF inhibitor-resistant cells exposed to BRAF inhibitor. Further confirmation of these mechanisms in a given BRAF inhibitor-resistant sample may be accomplished, for example, using sh/siRNA-mediated knockdown of upregulated RTKs or activated NRAS in the presence of BRAF inhibitor. Any significant levels of growth inhibition may indicate that upregulation of RTK or NRAS signaling is the cause of BRAF inhibition in that particular sample (Nazarian, et al., 2010).

Detecting reactivation of MAPK signaling in BRAF inhibitor-resistant cells may indicate another bypass mechanism for BRAF inhibitor resistance. COT and C-RAF have been shown to be upregulated in a BRAF V600E background exposed to BRAF inhibitor. Quantitative real-time RT-PCR, e.g., may reveal increased COT expression in BRAF inhibitor-resistant cells in the presence of BRAF inhibitor. Furthermore, sh/siRNA-mediated knockdown of COT in BRAF inhibitor-resistant cells in the presence of BRAF inhibitor may reduce the viability of BRAF inhibitor-resistant cells, indicating that these particular cells may be sensitive to COT inhibition and/or combination BRAF inhibitor/MEK inhibitor treatments (Johannessen, et al., 2010).

Reactivation of MAPK signaling may also be accomplished in a BRAF inhibitor-resistant background by activating mutations in MEK1. Targeted, massively parallel sequencing of genomic DNA from a BRAF inhibitor-resistant tumor may reveal activating mutations in MEK1, such as C121S, G128D, N122D, and Y130, among others. Other, undocumented mutations in MEK1 may be analyzed by, for example, expressing the particular mutation in a BRAF inhibitor-sensitive cell line such as A375. Determining levels of growth inhibition in these cells upon exposure to BRAF inhibitor may indicate if the MEK1 mutation is causing resistance to BRAF inhibitory therapy. To confirm such a finding, Western blotting for elevated levels of phospho-ERK1/2 in cells ectopically expressing the MEK1 mutation may indicate that the MEK1 mutation is allowing the BRAF inhibitor-resistant tumor to bypass BRAF and promote phosphorylation of ERK through MEK1 (Wagle, et al., 2011).

In accordance with the present invention, screening for a cancer that is refractory or resistant to MEK inhibitor therapy may comprise, e.g., identifying (i) amplification of mutant BRAF, (ii) STAT3 upregulation, (iii) mutations in the allosteric pocket of MEK that directly block binding of inhibitors to MEK or lead to constitutive MEK activity, and combinations thereof.

Amplification of mutant BRAF may cause MEK inhibitor resistance. MEK inhibitor resistance is typically associated with high levels of phosphorylated ERK and MEK in the presence of a MEK inhibitor, which may be assessed via, for example, Western blotting. Amplification of mutant BRAF in MEK inhibitor-resistant cell lines may be detected by, for example, fluorescence in situ hybridization (FISH) or quantitative PCR from genomic DNA of the resistant cell lines. Confirmation that BRAF amplification is a primary cause of MEK inhibitor resistance may entail using BRAF-targeted sh/siRNAs in resistant cells. If a significant decrease in MEK or ERK phosphorylation is observed, BRAF amplification may be a suitable target for further therapeutic approaches. (Corcoran, et al., 2010).

Identifying STAT3 upregulation may indicate that a particular tumor sample is resistant to MEK inhibitor therapy. Genome-wide expression profiling may reveal the STAT3 pathway to be upregulated in a tumor. Other techniques, such as Western blotting for phospho-STAT3 and real-time qPCR for the STAT pathway-associated genes JAK1 and IL6ST may reveal upregulated STAT3. Further confirmation that STAT3 upregulation causes MEK inhibitor resistance in a particular sample may comprise the use of sh/siRNAs against STAT3 in the sample followed by appropriate Western blotting for MEK and ERK activation as well as phospho-STAT3 and total STAT3. Growth inhibition studies may show that STAT3 knockdown sensitizes previously MEK inhibitor-resistant cells to MEK inhibition. A similar effect may be seen if the sample were exposed to a STAT3 inhibitor such as JSI-124. Additional confirmation that STAT3 upregulation is the cause of MEK inhibitor resistance in a particular tumor could arise from Western blotting for BIM expression, including BIM-EL, BIM-L, and BIM-SL. BIM expression leads to MEK inhibitor-induced apoptosis, thus STAT3 upregulation may lower BIM levels. STAT3 is known to regulate the expression of miR 17-92, which suppresses BIM expression. Upregulated STAT3 may lead to higher levels of miR 17-92, which will lower BIM levels and promote resistance to MEK inhibition. Thus, real-time qPCR of miR 17-92 levels may also assist in assessing whether STAT3 upregulation is causing MEK inhibition resistance in a particular sample. (Dai, et al., 2011).

Mutations in the allosteric pocket of MEK that can directly block binding of inhibitors to MEK or lead to constitutive MEK activity may be detected by methods disclosed below. Such mutations have been identified previously by Emery and colleagues (Emery, et al., 2009) as well as Wang and colleagues (Wang et al., 2011). Other mutations may affect MEK1 codons located within or abutting the N-terminal negative regulatory helix, such as P124L and Q56P. (Id.).

Methods for identifying mutations in nucleic acids, such as the above identified MEK genes, are known in the art. Nucleic acids may be obtained from biological samples. In the present invention, biological samples include, but are not limited to, blood, plasma, urine, skin, saliva, and biopsies. Biological samples are obtained from a subject by routine procedures and methods which are known in the art.

Non-limiting examples of methods for identifying mutations include PCR, sequencing, hybrid capture, in-solution capture, molecular inversion probes, fluorescent in situ hybridization (FISH) assays, and combinations thereof.

Various sequencing methods are known in the art. These include, but are not limited to, Sanger sequencing (also referred to as dideoxy sequencing) and various sequencing-by-synthesis (SBS) methods as disclosed in, e.g., Metzker 2005, sequencing by hybridization, by ligation (for example, WO 2005021786), by degradation (for example, U.S. Pat. Nos. 5,622,824 and 6,140,053) and nanopore sequencing (which is commercially available from Oxford Nanopore Technologies, UK). In deep sequencing techniques, a given nucleotide in the sequence is read more than once during the sequencing process. Deep sequencing techniques are disclosed in e.g., U.S. Patent Publication No. 20120264632 and International Patent Publication No. WO2012125848.

PCR-based methods for detecting mutations are known in the art and employ PCR amplification, where each target sequence in the sample has a corresponding pair of unique, sequence-specific primers. For example, the polymerase chain reaction-restriction fragment length polymorphism (PCR-RFLP) method allows for rapid detection of mutations after the genomic sequences are amplified by PCR. The mutation is discriminated by digestion with specific restriction endonucleases and is identified by electrophoresis. See, e.g., Ota et al., 2007. Mutations may also be detected using real time PCR. See, e.g., International Application publication No. WO2012046981.

Hybrid capture methods are known in the art and are disclosed in e.g., U.S. Patent Publication No. 20130203632 and U.S. Pat. Nos. 8,389,219 and 8,288,520. These methods are based on the selective hybridization of the target genomic regions to user-designed oligonucleotides. The hybridization can be to oligonucleotides immobilized on high or low density microarrays (on-array capture), or solution-phase hybridization to oligonucleotides modified with a ligand (e.g. biotin) which can subsequently be immobilized to a solid surface, such as a bead (in-solution capture).

Molecular Inversion Probe (MIP) techniques are known in the art and are disclosed in e.g., Absalan et al., 2008. This method uses MIP molecules, which are special “padlock” probes (Nilsson et al, 1994) for genotyping. A MIP molecule is a linear oligonucleotide that contains specific regions, universal sequences, restriction sites and a Tag (index) sequence (16-22 bp). A MIP hybridizes directly around the genetic marker/SNP of interest. The MIP method may also use a number of “padlock” probe sets that hybridize to genomic DNA in parallel (Hardenbol et al., 2003). In case of a perfect match, genomic homology regions are ligated by undergoing an inversion in configuration (as suggested by the name of the technique) and creating a circular molecule. After the first restriction, all molecules are amplified with universal primers. Amplicons are restricted again to ensure short fragments for hybridization on a microarray. Generated short fragments are labeled and, through a Tag sequence, hybridized to a cTag (complementary strand for index) on an array. After the formation of Tag-cTag duplex, a signal is detected.

The following Tables 1, 2, and 3 show the SEQ ID Nos. of representative nucleic acid and amino acid sequences of wild type BRAF, N-RAS, and MEK1 from various animals in the sequence listing. These sequences may be used in methods for identifying subjects with mutant BRAF, N-RAS, and MEK1 genotypes.

TABLE 1
BRAF sequences

| | polypeptide or | | |
|---|---|---|---|
| | nucleic acid | | Other |
| SEQ ID NO. | sequence | Organism | information |
| 1 | nucleic acid | human | |
| 2 | polypeptide | human | |
| 3 | nucleic acid | rat (Rattus | |
| | | norvegicus) | |
| 4 | polypeptide | rat (Rattus | |
| | | norvegicus) | |
| 5 | nucleic acid | mouse, Mus | |
| | | musculus | |
| 6 | polypeptide | mouse, Mus | |
| | | musculus | |
| 7 | nucleic acid | rabbit, | |
| | | Oryctolagus | |
| | | cuniculus | |
| 8 | polypeptide | rabbit, | |
| | | Oryctolagus | |
| | | cuniculus | |
| 9 | nucleic acid | guinea pig, Cavia | |
| | | porcellus | |
| 10 | polypeptide | guinea pig, Cavia | |
| | | porcellus | |
| 11 | nucleic acid | dog, Canis lupus | variant x1 |
| | | familiaris | |
| 12 | polypeptide | dog, Canis lupus | variant x1 |
| | | familiaris | |
| 13 | nucleic acid | dog, Canis lupus | variant x2 |
| | | familiaris | |
| 14 | polypeptide | dog, Canis lupus | variant x2 |
| | | familiaris | |
| 15 | nucleic acid | cat, Felis catus | |
| 16 | polypeptide | cat, Felis catus | |
| 17 | nucleic acid | cow, Bos taurus | variant X1 |
| 18 | polypeptide | cow, Bos taurus | variant X1 |
| 19 | nucleic acid | cow, Bos taurus | variant X2 |
| 20 | polypeptide | cow, Bos taurus | variant X2 |
| 21 | nucleic acid | cow, Bos taurus | variant X3 |
| 22 | polypeptide | cow, Bos taurus | variant X3 |
| 23 | nucleic acid | cow, Bos taurus | variant X4 |
| 24 | polypeptide | cow, Bos taurus | variant X4 |
| 25 | nucleic acid | cow, Bos taurus | variant X5 |
| 26 | polypeptide | cow, Bos taurus | variant X5 |
| 27 | nucleic acid | cow, Bos taurus | variant X6 |
| 28 | polypeptide | cow, Bos taurus | variant X6 |
| 29 | nucleic acid | cow, Bos taurus | variant X7 |
| 30 | polypeptide | cow, Bos taurus | variant X7 |
| 31 | nucleic acid | cow, Bos taurus | variant X8 |
| 32 | polypeptide | cow, Bos taurus | variant X8 |
| 33 | nucleic acid | cow, Bos taurus | variant X9 |
| 34 | polypeptide | cow, Bos taurus | variant X9 |
| 35 | nucleic acid | cow, Bos taurus | variant X10 |
| 36 | polypeptide | cow, Bos taurus | variant X10 |
| 37 | nucleic acid | cow, Bos taurus | variant X11 |
| 38 | polypeptide | cow, Bos taurus | variant X11 |
| 39 | nucleic acid | cow, Bos taurus | variant 2 |
| 40 | polypeptide | cow, Bos taurus | variant 2 |
| 41 | nucleic acid | horse, Equus | |
| | | caballus | |
| 42 | polypeptide | horse, Equus | |
| | | caballus | |
| 43 | nucleic acid | chicken, Gallus | |
| | | gallus | |
| 44 | polypeptide | chicken, Gallus | |
| | | gallus | |

TABLE 2
N-RAS sequences

| | polypeptide or | | |
|---|---|---|---|
| SEQ ID | nucleic acid | | Other |
| NO. | sequence | Organism | information |
| 45 | nucleic acid | human | |
| 46 | polypeptide | human | |
| 47 | nucleic acid | rat (Rattus norvegicus) | |
| 48 | polypeptide | rat (Rattus norvegicus) | |
| 49 | nucleic acid | mouse, Mus musculus | |
| 50 | polypeptide | mouse, Mus musculus | |
| 51 | nucleic acid | guinea pig, Cavia porcellus | |
| 52 | polypeptide | guinea pig, Cavia porcellus | |
| 53 | nucleic acid | guinea pig, Cavia porcellus | variant X1 |
| 54 | polypeptide | guinea pig, Cavia porcellus | variant X1 |
| 55 | nucleic acid | dog, Canis lupus familiaris | |
| 56 | polypeptide | dog, Canis lupus familiaris | |
| 57 | nucleic acid | cat, Felis catus | |
| 58 | polypeptide | cat, Felis catus | |
| 59 | nucleic acid | cow, Bos taurus | |
| 60 | polypeptide | cow, Bos taurus | |
| 61 | nucleic acid | chicken, Gallus gallus | |
| 62 | polypeptide | chicken, Gallus gallus | |

TABLE 3
MEK1 sequences

| | polypeptide or | |
|---|---|---|
| | nucleic acid | |
| SEQ ID NO. | sequence | Organism |
| 63 | nucleic acid | human |
| 64 | polypeptide | human |
| 65 | nucleic acid | rat (Rattus norvegicus) |
| 66 | polypeptide | rat (Rattus norvegicus) |
| 67 | nucleic acid | mouse, Mus musculus |
| 68 | polypeptide | mouse, Mus musculus |
| 69 | nucleic acid | rabbit, Oryctolagus cuniculus |
| 70 | polypeptide | rabbit, Oryctolagus cuniculus |
| 71 | nucleic acid | guinea pig, Cavia porcellus |
| 72 | polypeptide | guinea pig, Cavia porcellus |
| 73 | nucleic acid | dog, Canis lupus familiaris |
| 74 | polypeptide | dog, Canis lupus familiaris |
| 75 | nucleic acid | cat, Felis catus |
| 76 | polypeptide | cat, Felis catus |
| 77 | nucleic acid | cow, Bos taurus |
| 78 | polypeptide | cow, Bos taurus |
| 79 | nucleic acid | horse, Equus caballus |
| 80 | polypeptide | horse, Equus caballus |
| 81 | nucleic acid | chicken, Gallus gallus |
| 82 | polypeptide | chicken, Gallus gallus |

In another aspect of this embodiment, the method further comprises administering at least one additional therapeutic agent, preferably an inhibitor of the PI3K/Akt pathway, as disclosed herein.

A further embodiment of the present invention is a method for treating or ameliorating the effects of cancer in a subject, which cancer is refractory or resistant to BRAF inhibitor therapy, MEK inhibitor therapy, or both. The method comprises administering to the subject an effective amount of BVD-523 or a pharmaceutically acceptable salt thereof.

Suitable and preferred subjects are as disclosed herein. In this embodiment, the methods may be used to treat the cancers disclosed above, including those cancers with the mutational backgrounds, resistance profiles, and MAPK activity identified above. Methods of identifying such mutations are also as set forth above.

In a further aspect of this embodiment, the method further comprises administering to the subject at least one additional therapeutic agent, preferably an inhibitor of the PI3K/Akt pathway, as disclosed herein.

Another embodiment of the present invention is a method for identifying a subject having cancer who would benefit from therapy with an ERK inhibitor. The method comprises:
(a) obtaining a biological sample from the subject; and
(b) screening the sample to determine whether the subject has one or more of the following markers:
   (i) a switch between RAF isoforms,
   (ii) upregulation of RTK or NRAS signaling,
   (iii) reactivation of mitogen activated protein kinase (MAPK) signaling,
   (iv) the presence of a MEK activating mutation,
   (v) amplification of mutant BRAF,
   (vi) STAT3 upregulation,
   (vii) mutations in the allosteric pocket of MEK that directly block binding of inhibitors to MEK or lead to constitutive MEK activity,
      wherein the presence of one or more of the markers confirms that the subject's cancer is refractory or resistant to BRAF and/or MEK inhibitor therapy and that the subject would benefit from therapy with an ERK inhibitor, which is BVD-523 or a pharmaceutically acceptable salt thereof.

Suitable and preferred subjects are as disclosed herein. In this embodiment, the methods may be used to identify a subject having cancers disclosed above, including those cancers with the mutational backgrounds, resistance profiles, and MAPK activity identified above. Methods of identifying such mutations are also as set forth above.

In one aspect of this embodiment, the method further comprises administering BVD-523 or a pharmaceutically acceptable salt thereof to a subject having one or more of the markers. Preferably, the method additionally comprises administering to the subject having one or more of the markers at least one additional therapeutic agent, preferably an inhibitor of the PI3K/Akt pathway, as disclosed herein.

An additional embodiment of the present invention is a pharmaceutical composition for treating or ameliorating the effects of a cancer in a subject, which cancer is refractory or resistant to non-ERK MAPK pathway therapy. The composition comprises a pharmaceutically acceptable carrier or diluent and an effective amount of BVD-523 or a pharmaceutically acceptable salt thereof.

Suitable and preferred subjects and types of non-ERK MAPK pathway inhibitor therapy are as disclosed herein. In this embodiment, the pharmaceutical composition may be used to treat the cancers disclosed above, including those cancers with the mutational backgrounds, resistance profiles, and MAPK activity identified above. Methods of identifying such mutations are also as set forth above.

In one aspect of this embodiment, the pharmaceutical composition further comprises at least one additional therapeutic agent, preferably an inhibitor of the PI3K/Akt pathway, as disclosed herein.

Another embodiment of the present invention is a kit for treating or ameliorating the effects of a cancer in a subject, which cancer is refractory or resistant to non-ERK MAPK pathway therapy. This kit comprises any pharmaceutical composition according to the present invention packaged together with instructions for its use.

The kits may also include suitable storage containers, e.g., ampules, vials, tubes, etc., for each pharmaceutical composition and other reagents, e.g., buffers, balanced salt solutions, etc., for use in administering the pharmaceutical compositions to subjects. The pharmaceutical compositions and other reagents may be present in the kits in any convenient form, such as, e.g., in a solution or in a powder form. The kits may further include a packaging container, optionally having one or more partitions for housing the pharmaceutical composition and other optional reagents.

Suitable and preferred subjects and types of non-ERK MAPK pathway inhibitor therapy are as disclosed herein. In this embodiment, the kit may be used to treat the cancers disclosed above, including those cancers with the mutational backgrounds, resistance profiles, and MAPK activity identified herein. Methods of identifying such mutations are as set forth above.

In one aspect of this embodiment, the kit further comprises at least one additional therapeutic agent, preferably an inhibitor of the PI3K/Akt pathway, as disclosed herein.

Another embodiment of the present invention is a method for inhibiting phosphorylation of RSK in a cancer cell that is refractory or resistant to a non-ERK MAPK pathway inhibitor. The method comprises contacting the cancer cell with an effective amount of BVD-523 or a pharmaceutically acceptable salt thereof for a period of time sufficient for phosphorylation of RSK in the cancer cell to be inhibited. In this embodiment, “contacting” means bringing BVD-523 or a pharmaceutically acceptable salt thereof and optionally one or more additional therapeutic agents into close proximity to the cancer cells. This may be accomplished using conventional techniques of drug delivery to mammals, or in the in vitro situation by, e.g., providing BVD-523 or a pharmaceutically acceptable salt thereof and optionally other therapeutic agents to a culture media in which the cancer cells are located. In the ex vivo situation, contacting may be carried out by, e.g., providing BVD-523 or a pharmaceutically acceptable salt thereof and optionally other therapeutic agents to a cancerous tissue.

Suitable and preferred types of non-ERK MAPK pathway inhibitors are as disclosed herein. In this embodiment, effecting cancer cell death may be accomplished in cancer cells having various mutational backgrounds, resistance profiles, and MAPK activity as disclosed above. Methods of identifying such mutations are also as set forth above.

The methods of this embodiment, which may be carried out in vitro, ex vivo, or in vivo, may be used to effect cancer cell death, by e.g., killing cancer cells, in cells of the types of cancer disclosed herein.

In one aspect of this embodiment, greater than 50% of RSK phosphorylation is inhibited. In another aspect of this embodiment, greater than 75% of RSK phosphorylation is inhibited. In an additional aspect of this embodiment, greater than 90% of RSK phosphorylation is inhibited. In a further aspect of this embodiment, greater than 95% of RSK phosphorylation is inhibited. In another aspect of this embodiment, greater than 99% of RSK phosphorylation is inhibited. In an additional aspect of this embodiment, 100% of RSK phosphorylation is inhibited.

In a further aspect of this embodiment, the cancer cell is a mammalian cancer cell. Preferably, the mammalian cancer cell is obtained from a mammal selected from the group consisting of humans, primates, farm animals, and domestic animals. More preferably, the mammalian cancer cell is a human cancer cell.

In a further aspect of this embodiment, the contacting step comprises administering BVD-523 or a pharmaceutically acceptable salt to a subject from whom the cancer cell was obtained.

In the present invention, an “effective amount” or a “therapeutically effective amount” of a compound or composition disclosed herein is an amount of such compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a compound or composition according to the invention will be that amount of the composition, which is the lowest dose effective to produce the desired effect. The effective dose of a compound or composition of the present invention may be administered as two, three, four, five, six or more sub-doses, administered separately at appropriate intervals throughout the day.

A suitable, non-limiting example of a dosage of a BVD-523 and other anti-cancer agents disclosed herein is from about 1 mg/kg to about 2400 mg/kg per day, such as from about 1 mg/kg to about 1200 mg/kg per day, 75 mg/kg per day to about 300 mg/kg per day, including from about 1 mg/kg to about 100 mg/kg per day. Other representative dosages of such agents include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. The effective dose of BVD-523 and other anti-cancer agents disclosed herein, may be administered as two, three, four, five, six or more sub-doses, administered separately at appropriate intervals throughout the day.

The BVD-523, other inhibitors, and various other anti-cancer agents disclosed herein, or a pharmaceutical composition of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, BVD-523, other inhibitors, and various other anti-cancer agents disclosed herein, or a pharmaceutical composition of the present invention may be administered in conjunction with other treatments. BVD-523, other inhibitors, and various other anti-cancer agents disclosed herein, or a pharmaceutical composition of the present invention may be encapsulated or otherwise protected against gastric or other secretions, if desired.

The pharmaceutical compositions of the invention comprise one or more active ingredients in admixture with one or more pharmaceutically-acceptable diluents or carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, PA.).

Pharmaceutically acceptable diluents or carriers are well known in the art (see, e.g., Remington, The Science and Practice of Pharmacy (21^{st} Edition, Lippincott Williams and Wilkins, Philadelphia, PA.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

The pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable diluents or carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

The pharmaceutical compositions of the present invention for rectal or vaginal administration may be presented as a suppository, which may be prepared by mixing one or more active ingredient(s) with one or more suitable nonirritating diluents or carriers which are solid at room temperature, but liquid at body temperature and, therefore, will melt in the rectum or vaginal cavity and release the active compound. The pharmaceutical compositions of the present invention which are suitable for vaginal administration also include pessaries, tampons, creams, gels, pastes, foams or spray formulations containing such pharmaceutically-acceptable diluents or carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

The pharmaceutical compositions of the present invention suitable for parenteral administrations may comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid diluent or carrier, for example water for injection, immediately prior to use. Extemporaneous injection solutions and suspensions may be prepared from sterile powders, granules and tablets of the type described above.

The present invention provides treatment of cancer which is refractory or resistant to non-ERK MAPK pathway inhibitor therapy and discloses combinations shown to enhance the effects of ERK inhibitors. Herein, applicants have also shown that the combination of different ERK inhibitors is likewise synergistic. Therefore, it is contemplated that the effects of the combinations described herein can be further improved by the use of one or more additional ERK inhibitors. Accordingly, some embodiments of the present invention include one or more additional ERK inhibitors.

The present invention also provides a method of treating a subject having an unresectable or metastatic BRAF600 mutation-positive melanoma comprising administering to the subject 600 mg BID of BVD-523 or a pharmaceutically acceptable salt thereof.

In some embodiments of the invention, the mutation is a BRAFV600E mutation.

The present invention also provides a composition for treating a subject having an unresectable or metastatic BRAF600 mutation-positive melanoma, the composition comprising 600 mg of BVD-523 or a pharmaceutically acceptable salt thereof and optionally a pharmaceutically acceptable carrier, adjuvant, or vehicle.

The following examples are provided to further illustrate the methods of the present invention. These examples are illustrative only and are not intended to limit the scope of the invention in any way.

### EXAMPLES

### Example 1

### Materials and Methods

Cancer cell lines were maintained in cell culture under standard media and serum conditions. For dose escalation studies, A375 cells were split, grown to about 40-60% confluence, and then treated with the initial dose of the specified drug. Table 4 shows a summary of drug treatments that were escalated.

TABLE 4
Summary of Treatments Being Escalated

| Treatment | Inhibitor |
|---|---|
| 1 | Trametinib (MEKi) |
| 2 | Dabrafenib (BRAFi) |
| 3 | BVD-523 (ERKi) |
| 4 | Dabrafenib (BRAFi) + Trametinib (MEKi) |
| 5 | Dabrafenib (BRAFi) + BVD-523 (ERKi) |
| 6 | Trametinib (MEKi) + BVD-523 (ERKi) |

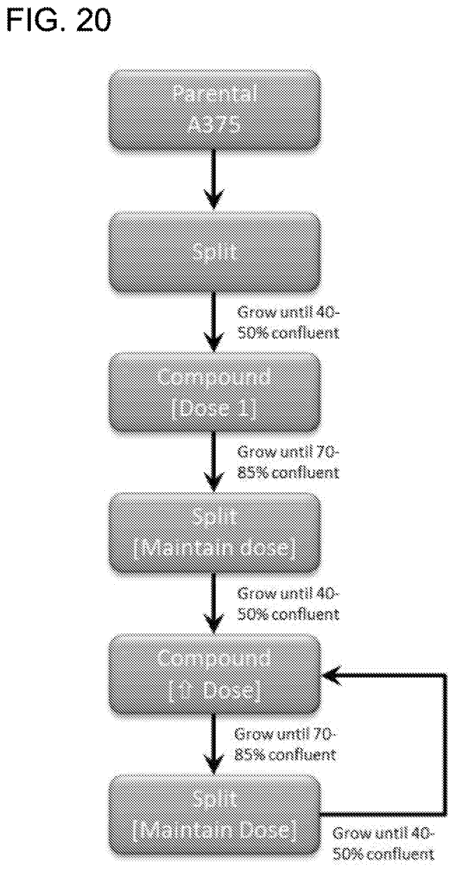

Single agent dose escalations were performed based on Little et al., 2011 and are outlined in FIG. 20. Cells were then allowed to grow until 70-90% confluence and split. Split ratios were kept as “normal” as possible and reasonably consistent between treatments (e.g. a minimum of 50% of the normal split ratio of the parentals). Medium was refreshed every 3-4 days. When cells again reached about 40-60% confluence, the dose was escalated. In the event that the 40-60% window was missed, the cells were split again and dosed once they reached 40-60% confluence. Again, medium was refreshed every 3-4 days. The process was repeated as required (FIG. 20).

For single agent treatments, starting concentrations and dose increases were conducted by starting with the approximate IC50, escalating in small increments or, gently, for the initial 4-5 doses, doubling the dose, increasing by the same increment for the next 4 doses, then moving to 1.5-fold increases in concentration for subsequent doses.

For combination treatments, starting concentrations and dose increases were conducted by starting with half of the approximate IC50 of each compound (combination assay suggests this will result in about 40-70% inhibition range), escalating as per single agents (i.e. doing an initial doubling and then increasing by the same increment for the next 4 doses, then moving to 1.5-fold increases in concentration). Table 5 shows the projected dose increases using these schemes.

TABLE 5
Projected Dose Increases - Month 1

| | | | | Dab/Tram | | Dab/523 | | Tram/523 | |
|---|---|---|---|---|---|---|---|---|---|
| | Tram | Dab | BVD-523 | Dab | Tram | Dab | 523 | Tram | 523 |
| Dose | (nM) | (nM) | (μM) | (nM) | (nM) | (nM) | (μM) | (nM) | (μM) |
| 1 | 1 | 5 | 0.16 | 2.5 | 0.5 | 2.5 | 0.08 | 0.5 | 0.08 |
| 2 | 2 | 10 | 0.32 | 5 | 1 | 5 | 0.16 | 1 | 0.16 |
| 3 | 3 | 15 | 0.48 | 7.5 | 1.5 | 7.5 | 0.24 | 1.5 | 0.24 |
| 4 | 4 | 20 | 0.64 | 10 | 2 | 10 | 0.32 | 2 | 0.32 |
| 5 | 5 | 25 | 0.80 | 12.5 | 2.5 | 12.5 | 0.40 | 2.5 | 0.40 |
| 6 | 8 | 38 | 1.2 | 19 | 4 | 19 | 0.6 | 4 | 0.6 |
| 7 | 11 | 56 | 1.8 | 28 | 6 | 28 | 0.9 | 6 | 0.9 |
| 8 | 17 | 84 | 2.7 | 42 | 8 | 42 | 1.4 | 8 | 1.4 |
| 9 | 25 | 127 | 4.1 | 63 | 13 | 63 | 2.0 | 13 | 2.0 |
| 10 | 38 | 190 | 6.1 | 95 | 19 | 95 | 3.0 | 19 | 3.0 |
| 11 | 57 | 285 | 9.1 | 142 | 28 | 142 | 4.6 | 28 | 4.6 |
| 12 | 85 | 427 | 13.7 | 214 | 43 | 214 | 6.8 | 43 | 6.8 |
| 13 | 128 | 641 | 20.5 | 320 | 64 | 320 | 10.3 | 64 | 10.3 |
| 14 | 192 | 961 | 30.8 | 481 | 96 | 481 | 15.4 | 96 | 15.4 |
| 15 | 288 | 1442 | 46.1 | 721 | 144 | 721 | 23.1 | 144 | 23.1 |
| 16 | 432 | 2162 | 69.2 | 1081 | 216 | 1081 | 34.6 | 216 | 34.6 |
| 17 | 649 | 3244 | 103.8 | 1622 | 324 | 1622 | 51.9 | 324 | 51.9 |
| 18 | 973 | 4865 | 155.7 | 2433 | 487 | 2433 | 77.8 | 487 | 77.8 |
| 19 | 1460 | 7298 | 233.5 | 3649 | 730 | 3649 | 116.8 | 730 | 116.8 |
| 20 | 2189 | 10947 | 350.3 | 5474 | 1095 | 5474 | 175.2 | 1095 | 175.2 |

Clonal resistant cell populations were derived from resistant cell pools by limiting dilution.

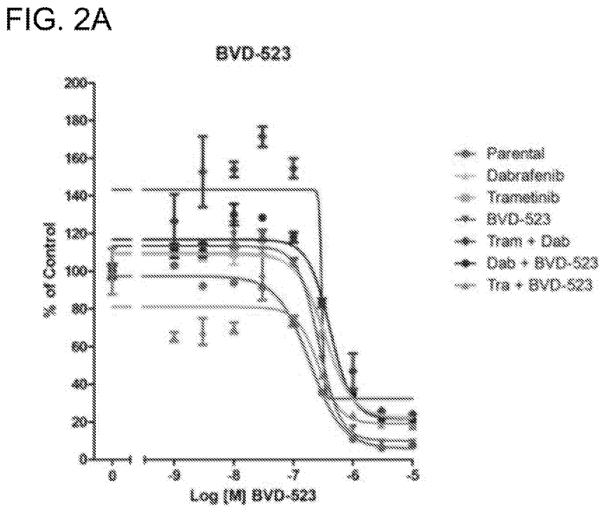

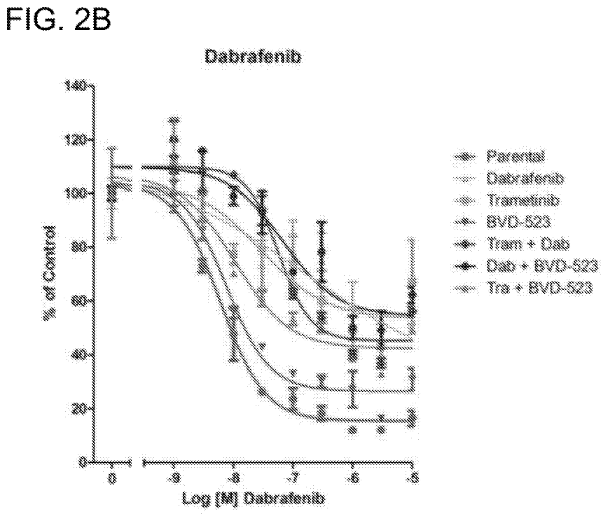

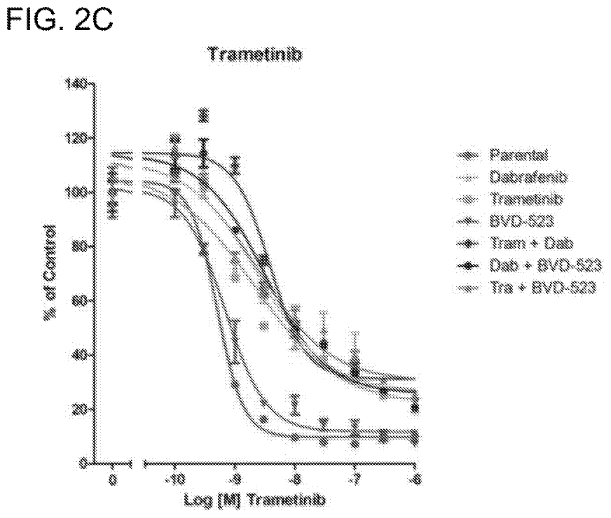

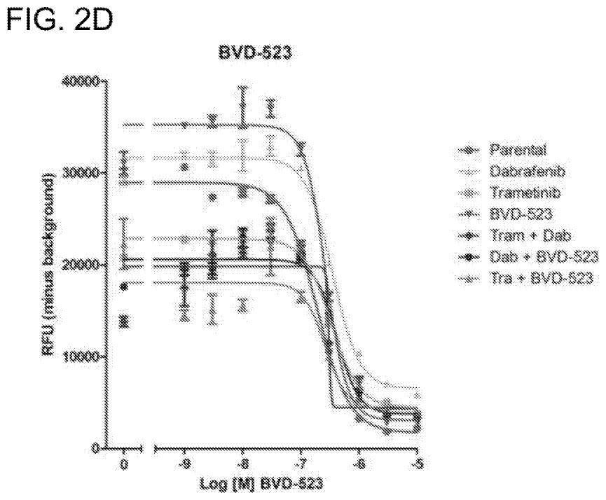

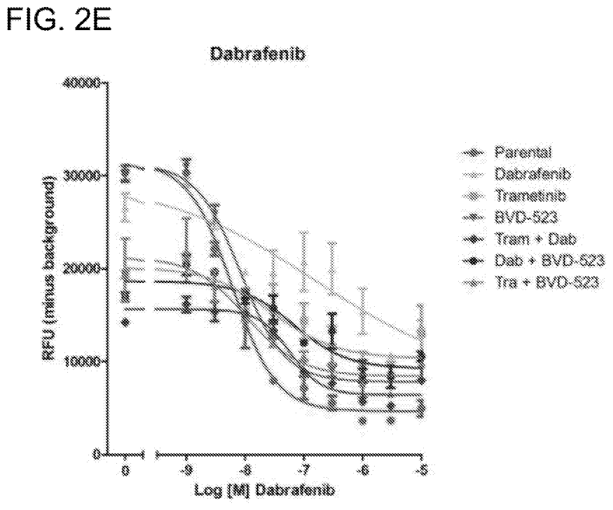

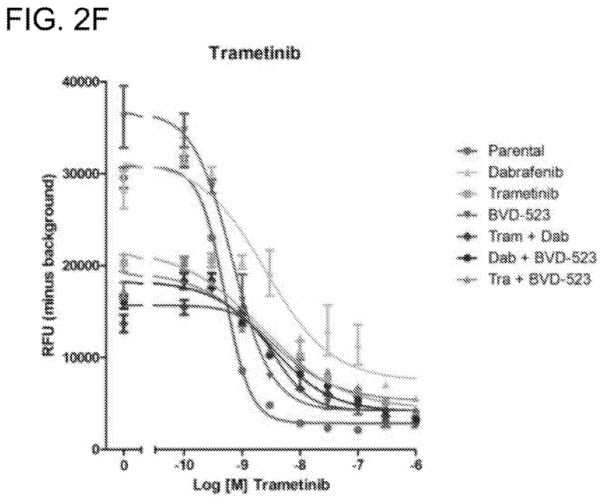

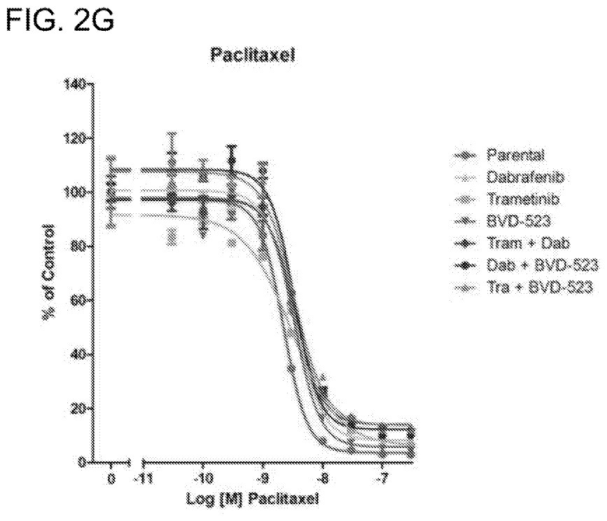

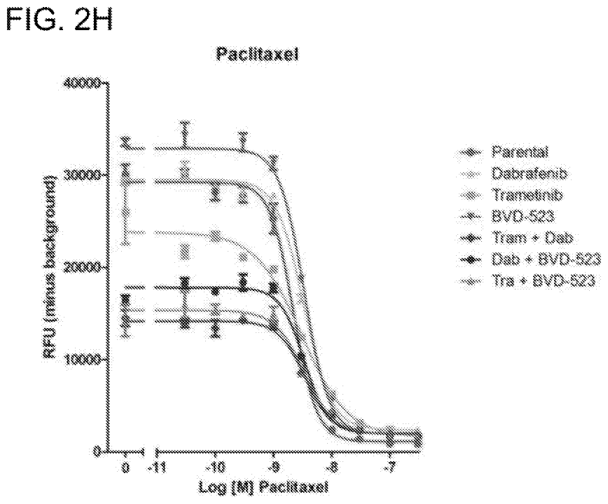

Proliferation assays were used to track changes in sensitivity to the escalated agent(s) at appropriate time intervals (e.g. each month, although the timing is dependent on adequate cell numbers being available). For proliferation assays, cells were seeded in 96-well plates at 3000 cells per well in drug-free DMEM medium containing 10% FBS and allowed to adhere overnight prior to addition of compound or vehicle control. Compounds were prepared from DMSO stocks to give a final concentration range as shown in FIG. 2A-FIG. 2H. The final DMSO concentration was constant at 0.1%. Test compounds were incubated with the cells for 96 hours at 37° C. and 5% CO₂ in a humidified atmosphere. Alamar Blue 10% (v/v) was then added and incubated for 4 hours and fluorescent product was detected using a BMG FLUOstar plate reader. The average media only background value was deducted and the data analyzed using a 4-parameter logistic equation in GraphPad Prism. Paclitaxel was used as a positive control.

Proliferation assays for month 1 were initiated at day 28 using cells growing in the concentrations of each agent indicated in Table 6.

TABLE 6
Initial Concentrations of Drugs Used in
Proliferation Assays - Month 1

| Line | Dab | Tram | BVD-523 |
|---|---|---|---|
| Parental | — | — | — |
| Tram | — | 2 nM | — |
| Dab | 15 nM | — | — |
| BVD-523 | — | — | 0.48 μM |
| Tram + Dab | 5 nM | 1 nM | — |
| Dab + BVD-523 | 7.5 nM | — | 0.24 μM |
| Tram + BVD-523 | — | 1 nM | 0.16 μM |

Proliferation assays for month 2 were initiated at day 56 using cells growing in the concentrations of each agent indicated in Table 7.

TABLE 7
Initial Concentrations of Drugs Used in
Proliferation Assays - Month 2

| Line | Dab | Tram | BVD-523 |
|---|---|---|---|
| Parental | — | — | — |
| Tram | — | 8 nM | — |
| Dab | 127 nM | — | — |
| BVD-523 | — | — | 0.8 μM |
| Tram + Dab | 10 nM | 2 nM | — |
| Dab + BVD-523 | 12.5 nM | — | 0.4 μM |
| Tram + BVD-523 | — | 2 nM | 0.32 μM |

At the end of the 3 month escalation period, cultures were maintained at the top concentration for 2 weeks prior to the final round of proliferation assays and potential single cell cloning. As the proliferation assays/single cell cloning required actively proliferating cells, for treatments where cells were proliferating very slowly at the top concentration or that were only recently escalated, a backup culture was also maintained at a lower concentration (Table 8). For the BVD-523 treatment, where cells appeared to have almost completely stopped growing and looked particularly fragile at the top concentration (1.8 μM), cultures were maintained at a lower concentration for the 2 week period.

TABLE 8
Details of Treatments Being Cultured at a
Fixed Concentration for 2 Weeks

| | | | Backup |
|---|---|---|---|
| Treatment | Inhibitor | Culture 1 | Culture |
| 1 | Tram | 160 nM | 80 nM |
| 2 | Dab | 3.2 μM | — |
| 3 | BVD-523 | 1.2 μM | 0.8 μM |
| 4 | Dab + Tram | D: 160 nM | D: 80 nM |
| | | T: 30 nM | T: 16 nM |
| 5 | Dab + | D: 42 nM | D: 28 nM |
| | BVD-523 | 523: 1.4 μM | 523: 0.9 μM |
| 6 | Tram + | T: 4 nM | T: 2.5 nM |
| | BVD-523 | 523: 0.6 μM | 523: 0.4 μM |

Proliferation assays for month 3 used cells growing in the concentrations of each agent indicated in Table 9.

TABLE 9
Initial Concentrations of Drugs Used
in Proliferation Assays - Month 3

| Line | Dab | Tram | BVD-523 |
|---|---|---|---|
| Parental | — | — | — |
| Tram | — | 160 nM | — |
| Dab | 3.2 μM | — | — |
| BVD-523 | — | — | 1.2 μM |
| Tram + Dab | 80 nM | 16 nM | — |
| Dab + BVD-523 | 28 nM | — | 0.9 μM |
| Tram + BVD-523 | — | 2.5 nM | 0.4 μM |

For combination studies, A375 cells (ATCC) were seeded into triplicate 96-well plates at a cell density of 3000 cells/well in DMEM plus 10% FBS and allowed to adhere overnight prior to addition of test compound or vehicle control. Combinations were tested using a 10×8 dose matrix with a final DMSO concentration of 0.2%. A 96 hour assay incubation period followed, with subsequent addition of Alamar Blue 10% (v/v) and 4 hours incubation prior to reading on a fluorescent plate reader. After reading Alamar Blue, the medium/Alamar Blue mix was flicked off and 100 μl of CellTiter-Glo/PBS (1:1) added and the plates processed as per the manufacturers instructions (Promega). Media only background values were subtracted before the data was analysed. The Bliss additivity model was then applied.

In brief, predicted fractional inhibition values for combined inhibition were calculated using the equation C_{bliss}=A+B−(A×B) where A and B are the fractional inhibitions obtained by drug A alone or drug B alone at specific concentrations. C_{bliss} is the fractional inhibition that would be expected if the combination of the two drugs were exactly additive. C_{bliss} values are subtracted from the experimentally observed fractional inhibition values to give an ‘excess over Bliss’ value. Excess over Bliss values greater than 0 indicate synergy, whereas values less than 0 indicate antagonism. Excess over Bliss values are plotted as heat maps±SD.

The single and combination data are also presented as dose-response curves generated in GraphPad Prism (plotted using % viability relative to DMSO only treated controls).

For focused combination studies, the Alamar Blue viability assays were performed as described above for combination studies. Additionally, Caspase-Glo 3/7 assays were performed. In brief, HCT116 cells were seeded in triplicate in white 96-well plates at a cell density of 5000 cells/well in McCoy's 5A plus 10% FBS. A375 cells were seeded at a density of 5000 cells/well in DMEM plus 10% FBS. Cells were allowed to adhere overnight prior to addition of test compound or vehicle control. The final concentration of DMSO was 0.2%, and 800 nM staurosporine was included as a positive control. 24 and 48 hour assay incubation periods were used. Then, Caspase-Glo® 3/7 50% (v/v) was added, plates were mixed for 5 minutes on an orbital shaker and incubated for 1 hour at room temperature prior to reading on a luminescent plate reader. Media only background values were subtracted before the data was analysed.

For Differential Scanning Fluorimetry, SYPRO orange (5,000× solution, Invitrogen) was diluted (1:1,000) in buffer solution (10 mM HEPES, 150 mM NaCl, pH 7.5). His×6 tagged proteins included inactive ERK2, active ERK2 (ppERK2), or p38a at a final concentration of 1 μM. The protein/dye solution and compounds in 100% DMSO were added to wells (2% v/v final DMSO concentration) to achieve the desired final concentrations, mixed, and placed into an RT-PCR instrument. Next, a melting curve was run from 25-95° C. at a rate of 1° C. per minute and the melting temperature (Tm) was determined for each protein in the absence or presence of compounds. The change in Tm (ΔTm) in the presence of various drug concentrations is presented.

For Ki determination of ERK1, activated ERK1 (10 nM) was incubated with various concentrations of the compounds in 2.5% (v/v) DMSO for 10 minutes at 30° C. in 0.1 M HEPES buffer (pH 7.5), 10 mM MgCl₂, 2.5 mM phosphoenolpyruvate, 200 UM nicotinamide adenine dinucleotide (NADH), 150 μg/mL pyruvate kinase, 50 μg/mL lactate dehydrogenase, and 200 μM Erktide peptide. The reaction was initiated by the addition of 65 UM of ATP. Decreased absorbance rate (340 nm) was monitored and the IC₅₀ was determined as a function of inhibitor concentration.

For Ki determination of ERK2, the inhibitory activity of BVD-523 against ERK2 was determined using a radiometric assay, with final concentration of the components being 100 mM HEPES (pH 7.5), 10 mM MgCl₂, 1 mM dithiothreitol (DTT), 0.12 nM ERK2, 10 UM myelin basic protein (MBP), and 50 μM ³³P-γ-ATP. All reaction components, with the exception of ATP and MBP, were premixed and aliquoted (33 μL) into a 96-well plate. A stock solution of compound in DMSO was used to make up to 500-fold dilutions; a 1.5-μL aliquot of DMSO or inhibitor in DMSO was added to each well. The reaction was initiated by adding the substrates ³³P-γ-ATP and MBP (33 μL). After 20 minutes the reaction was quenched with 20% (w/V) tricholoracetic acid (TCA) (55 μL) containing 4 mM ATP, transferred to the GF/B filter plates, and washed 3 times with 5% (w/v) TCA). Following the addition of Ultimate Gold™ scintillant (50 μL), the samples were counted in a Packard TopCount. From the activity versus concentration titration curve, the Ki value was determined by fitting the data to an equation for competitive tight binding inhibition kinetics using Prism software, version 3.0.

For IC50 determination of ERK2, activity was assayed by a standard coupled-enzyme assay. The final concentrations were as follows: 0.1 M HEPES (pH 7.5), 10 mM MgCl₂, 1 mM DTT, 2.5 mM phosphoenolpyruvate, 200 μM NADH, 50 μg/mL pyruvate kinase, 10 μg/mL lactate dehydrogenase, 65 μM ATP, and 800 μM peptide (ATGPLSPGPFGRR). All of the reaction components except ATP were premixed with ERK and aliquoted into assay-plate wells. BVD-523 in DMSO was introduced into each well, keeping the concentration of DMSO per well constant. BVD-523 concentrations spanned a 500-fold range for each titration. The assay-plate was incubated at 30° C. for 10 minutes in the plate reader compartment of the spectrophotometer (molecular devices) before initiating the reaction by adding ATP. The absorbance change at 340 nm was monitored as a function of time; the initial slope corresponds to the rate of the reaction. The rate versus concentration of the BVD-523 titration curve was fitted either to an equation for competitive tight-binding inhibition kinetics to determine a value for Ki or to a 3-parameter fit to determine the IC₅₀ using Prism software, version 3.0.

For apoptosis assays, cells were plated at 2×10⁴ cells per well in a 96-well plate and allowed to attach overnight or grow to 50% confluency. Cells were treated with a serial dilution of BVD-523 in media (final volume 200 μL, concentration ranges 4-0.25 UM) and incubated for 48 hours in a 37° C. CO₂ incubator. Cells were washed with 100 μL of PBS, and 60 μL of radioimmunoprecipitation assay buffer was added (50 mM Tris-HCl, pH 8.0, 150 mM NaCl, 1.0% [w/v] NP-40, 0.5% [w/v] sodium deoxycholate, 1% [w/v] SDS), then incubated for 10 minutes at 4° C. to lyse the cells. A 30-μL lysate aliquot was added to 100 μL of caspase assay buffer (120 mM HEPES, 12 mM EDTA, 20 mM dithiothreitol, 12.5 μg/mL AC-DEVD-AMC caspase substrate) and incubated at RT from 4 hours to overnight. The plate was read in a fluorimeter (excitation wavelength 360 nm, emission wavelength 460 mm). The remaining 30 μL of lysate was analyzed for total protein content using the BioRad Protein Assay Kit (sample-to-working reagent ratio of 1:8). Final normalized caspase activity was derived as fluorescence units per μg protein and converted to a fold increase in caspase activity when compared with DMSO controls.

For measurement of antitumor activity in A375 xenografts, xenografts were initiated with A375 cells maintained by serial subcutaneous transplantation in female athymic nude mice. Each test mouse received an A375 tumor fragment (1 mm³) implanted subcutaneously in the right flank. Once tumors reached target size (80-120 mm³), animals were randomized into treatment and control groups, and drug treatment was initiated.

To evaluate BVD-523 monotherapy, BVD-523 in 1% (w/v) carboxymethylcellulose (CMC) was administered orally, per os (p.o.), BID at doses of 5, 25, 50, 100, or 150 mg/kg. Oral temozolomide was administered as a positive reference compound at 75 or 175 mg/kg once daily (QD) for a total of five treatments (QD×5).

The efficacy of BVD-523 in combination with dabrafenib was evaluated in mice randomized into 9 groups of 15 and 1 group of 10 (Group 10). Dabrafenib was administered p.o. at 50 or 100 mg/kg QD and BVD-523 was administered p.o. at 50 or 100 mg/kg BID, alone and in combination, until study end; vehicle-treated and temozolomide-treated (150 mg/kg QD×5) control groups were also included. Combination dosing was stopped on Day 20 to monitor for tumor regrowth. Animals were monitored individually and euthanized when each tumor reached an endpoint volume of 2000 mm³, or the final day (Day 45), whichever came first, and median time to endpoint (TTE) calculated. The combination was also evaluated in an upstaged A375 model where larger tumors in the range 228-1008 mm³ were evaluated. Here, mice were randomized into 1 group (Group 1) of 14 and 4 groups (Groups 2-5) of 20. Dosing was initiated on Day 1 with dabrafenib plus BVD-523 (25 mg/kg dabrafenib+50 mg/kg BVD-523 or 50 mg/kg dabrafenib+100 mg/kg BVD-523), with each agent given p.o. BID until study end. The study included 50-mg/kg dabrafenib and 100-mg/kg BVD-523 monotherapy groups as well as a vehicle-treated control group. Tumors were measured twice weekly. Combination dosing was stopped on Day 42 to monitor for tumor regrowth through study end (Day 60). Treatment outcome was determined from % TGD, defined as the percent increase in median TTE for treated versus control mice, with differences between groups analyzed via log rank survival analysis. For TGI analysis, % TGI values were calculated and reported for each treatment (T) group versus the control (C) using the initial (i) and final (f) tumor measurements based on the following formula: % TGI=1−Tf−Ti/Cf−C. Mice were also monitored for CR and PR responses. Animals with a CR at the end of the study were additionally classified as TFS.

For measurement of BVD-523 activity in Colo205 xenografts, human Colo205 cells were cultured in RPMI 1640 supplemented with 10% (v/v) fetal bovine serum (FBS), 100 units/mL penicillin, 100 μg/mL streptomycin (Invitrogen), and 2 mM L-glutamine. Cells were cultured for fewer than four passages prior to implantation. Female athymic nude mice (19-23 g) were injected subcutaneously with 2×10⁶ Colo205 cells into the right dorsal axillary region on Day 0.

Mice with an approximate tumor volume of 200 mm³ were randomized into 6 experimental groups. Vehicle control, 1% CMC (w/v), was prepared weekly. BVD-523 was suspended in 1% (w/v) CMC at the desired concentration and homogenized on ice at 6,500 rpm for 50 minutes. BVD-523 suspensions were prepared weekly and administered p.o. BID at total daily doses of 50, 100, 150, and 200 mg/kg (n=12/group) on an 8- or 16-hour dosing schedule for 13 days. The vehicle control (n=12) was administered using the same dosing regimen. CPT-11 was administered as a positive reference compound (n=12). Each 1 mL of CPT-11 injection contained 20 mg irinotecan, 45 mg sorbitol, and 0.9 mg lactic acid. CPT-11 was administered at 100 mg/kg/day intraperitoneally every 4 days for 2 consecutive doses.

For measurement of ERK1/2 Isotope-Tagged Internal Standard (ITIS) Mass Spectrometry in Colo205 Xenografts, frozen tumors were lysed in 10 volumes of ice cold lysis buffer (10 mM TRIS-HCl, pH 8.0, 10 mM MgCl₂, 1% (v/v) Triton X-100, Complete™ Protease Inhibitor Cocktail [Roche, cat. No. 1836170], Phosphatase Inhibitor Cocktail I [Sigma, cat. No. P-2850], Phosphatase Inhibitor Cocktail II [Sigma cat. No. 5726], and benzonase [Novagen cat. No. 70664]). Lysates were clarified by centrifugation (100,000×g for 60 minutes at 4° C.) and the supernatants adjusted to 2 mg/mL with lysis buffer. ERK1 was immunoprecipitated using agarose-coupled and pan-anti-ERK1 (Santa Cruz Biotechnology cat. No. sc-93ac) antibodies. Immunoprecipitated proteins were resolved by SDS-PAGE and stained with SYPRO Ruby (Invitrogen), and the ERK bands excised via razor. Gel slices were washed in 300 μL of 20 mM NH₄HCO₃, diced into small pieces, and placed in Page Eraser Tip (The Nest Group cat no. SEM0007). Gel fragments were reduced and alkylated prior to trypsin digestion. Tryptic fragments were isolated in 75 μL of 50% (v/v) Acetonitrile, 0.2% (v/v) trifluoroacetic acid and the resulting sample concentrated to 0-10 μL in a SpeedVac.

For ITIS analysis, digested samples were spiked with heavy-atom labeled peptide standards and fractional phosphorylation was quantified by coupled liquid chromatography-tandem mass spectrometry (MS). Nanocapillary chromatography was performed using a Rheos 2000 binary pump from Flux Instruments delivering nanoscale flow after 1:750 splitting, an LC Packings Inertsil nano-precolumn (C18, 5 mm, 100 Å, 30 mm ID×1 mm), and a New Objective PicoFrit AQUASIL resolving column (C18, 5 mm, 75/15 mm ID×10 cm), which also served as an electrospray ionization (ESI) emitter. An Applied Biosystem API 3000 mass spectrometer coupled with a nano-ESI source was used for MS analysis. An in-house-made gas nozzle connected to a nebulizing gas source was used to help steady nano-flow spray. Data were acquired in a multiple reaction monitoring (MRM) mode: nebulizing gas at 3; curtain gas at 7; collision gas at 5; ion spray voltage at 2150 volts, exit potential at 10 volts; Q1/Q3 resolution Low/Unit; and dwell time of 65 msec for all MRM channels. All raw MS data were processed using a combination of the Analyst software suite from Applied Biosystem and custom tools.

For assessment of drug sensitivity in cell-line models of acquired resistance, drug sensitivity of dose-escalated A375 cells and isogenic RKO cells was assessed in 96-hour proliferation assays. RKO isogenic cells (McCoy's 5A containing 10% [v/v] FBS) or dose-escalated A375 cells (DMEM containing 10% FBS were seeded into 96-well plates and allowed to adhere overnight prior to addition of compound or vehicle control. Note that the dose-escalated A375 cells were seeded in the absence of inhibitor. Compounds were prepared from 0.1% (v/V) DMSO stocks to give a final concentration as indicated. Test compounds were incubated with the cells for 96 hours at 37° C. in a 5% CO₂ humidified atmosphere. For the RKO cells, CellTiter-Glo® reagent (Promega) was added according to manufacturer's instructions and luminescence detected using a BMG FLUOstar plate reader. For the A375 assays Alamar blue (ThermoFisher) 10% (v/v) was added and incubated for 4 h, and fluorescent product was then detected using a BMG FLUOstar. The average media only background value was deducted and the data analyzed using a 4-parameter logistic equation in GraphPad Prism.

IC₅₀ Determination of ERK1 was measured in a final reaction volume of 25 μL. ERK1 (human) (5-10 mU) was incubated with 25 mM Tris (pH 7.5), 0.02 mM ethyleneglycoltetracetic acid, 250 UM peptide, 10 mM Mg acetate, and γ-33P-ATP (specific activity approximately 500 cpm/pmol, concentration as required). Adding Mg ATP initiated the reaction. After incubation for 40 minutes at room temperature (RT), the reaction was stopped by adding 5 μL of a 3% (w/v) phosphoric acid solution. Then, 10 μL of the reaction was spotted onto a P30 filtermat, and washed 3 times for 5 minutes in 75 mM of phosphoric acid then once in methanol before drying and scintillation counting.

RKO MEK1 Q56P Isogenic cells were produced by Horizon Discovery (Cambridge, UK; #HD 106-019) using a recombinant AAV-mediated gene targeting strategy. Briefly, rAAV virus was generated following transfection of the appropriate targeting vector and helper vectors in HEK293T cells, purified using an AAV purification kit (Virapur, San Diego, USA) and titrated using qPCR. Parental homozygous RKO cells (homozygous wild type for MEK1) were then infected with rAAV virus and clones that had integrated the selection cassette were identified by G418 selection and expanded. Correctly targeted clones that were heterozygous for knock-in of the MEK1 Q56P point mutation into a single allele were identified by PCR and sequencing.

Isogenic SW48 cell lines heterozygous for knock-in of mutant KRAS (De Roock et al 2010, JAMA, 304, 1812-1820) were obtained from Horizon Discovery (Catalogue numbers; HD 103-002, HD 103-006 HD 103-007, HD 103-009, HD 103-010, HD 103-011, HD 103-013). For proliferation assay, cells were seeded into 96-well plates in McCoy's 5A medium supplemented with 10% FBS and allowed to adhere overnight prior to addition of compound or vehicle control. Test compounds were incubated with the cells for 96 hours at 37° C. in a 5% CO₂ atmosphere. Viability was then assessed using Alamar blue.

The proprietary KinaseProfiler assay was conducted at Upstate Discovery and employed radiometric detection similar to that employed by Davies et al, was used to profile the selectivity of BVD-523 against a panel of 70 kinases.

A drug sensitivity analysis was carried out as part of The Genomics of Drug Sensitivity in Cancer Project using high-throughput screening, as previously described (Yang et al. 2013).

For Western blot analysis, A375 cells were seeded onto 10 cm dishes in Dulbecco's Modified Eagle's Medium plus 10% (v/v) FBS. Cells were allowed to adhere overnight prior to the addition of test compound or vehicle. For experiments with RKO cells, these cells were seeded in 6-well plates or 10 cm dishes with McCoy's 5A+10% (v/v) FBS. Cells were then treated at the desired concentration and duration. Cells were harvested by trypsinization, pelleted, and snap frozen. Lysates were prepared with RIPA buffer supplemented with protease and phosphatase inhibitor cocktails (Roche), clarified by centrifugation at 11,000 rpm for 10 minutes, and quantitated by bicinchoninic acid assay. Samples were resolved by SDS-PAGE, blotted onto polyvinylidene difluoride membranes, and probed using antibodies (i.e., pRB [Ser780], cat. no. 9307; CCND1, cat. no. ab6152; BCL-xL, cat. no. 2762; PARP, cat. no. 9542; DUSP6, cat. no. 3058S) directed to the indicated targets.

For Reverse Phase Protein Analysis (RPPA), A375, MIAPaCa-2, HCT116, Colo205, HT-29, and AN3Ca cells (ATCC) were plated at 80% confluence, allowed to recover overnight (MIAPaCa-2 cells were plated at 30% confluence and allowed to recover for 3 days), then treated with 10 UM of each compound (i.e., BVD-523, SCH722984, GDC-0994, or Vx-Ile) for 6 hours at 37° C. Control wells were treated with DMSO at 0.1% (v/v) for 6 hours prior to cell lysate generation. Samples were then analyzed using reverse-phase protein microarray technology (Theranostics Health).

For analysis of pERK IHC in Colo205 xenografts, xenograft tumors were processed overnight in 70% through 100% graded ethanols, cleared in two changes of xylene, infiltrated with paraffin, and embedded into paraffin blocks. Then, 5-μm sections were cut and placed onto positively charged glass slides and baked for at least 30 minutes, but not longer than 1 hour, at 60° C. A single section from each animal and dose group was probed with anti-phospho p42/p44 MAPK antibody (pERK [1:100], CST; Cat no. 9101; Lot no. 16), counterstained with hematoxylin, and then analyzed microscopically using a Zeiss Axioplan 2 microscope. An isotype control (rabbit, Zymed laboratories, catalog no. 08-6199, lot no. 40186458) was run as a negative control.

For FACS analysis, cells were scraped and pelleted at 1,500 rpm for 5 minutes, then re-suspended in 1 mL of buffer and frozen at −70° C. The frozen cells were thawed and centrifuged again, followed by 10 minutes of re-suspension in 0.25 mL of Buffer A (trypsin in spermine tetrahydrochloride detergent buffer) to disaggregate cell clumps and digest cell membranes and cytoskeletons. Buffer B (trypsin inhibitor and Ribonuclease I in buffer, 0.2 mL) was added for 10 minutes in the dark. The resulting DNA-stained nuclei were filtered and analyzed by FACS. The histograms were analyzed to establish the proportion of cells in the G1, S, and G2/M phases of the cell cycle based on the presence of n and 2n DNA (or higher) content.

For measurement of in vitro combination activity, five thousand G-361 cells were seeded into triplicate 96-well plates containing McCoy's 5A with 10% (v/v) FBS and allowed to adhere overnight. The vemurafenib/BVD-523 combination was tested using a 10×8 dose matrix. Compounds were incubated with the cells for 72 hours at 37° C. in a 5% CO₂ humidified atmosphere. CellTiter-Glo reagent was added according to manufacturer's instructions and luminescence detected using a MBG FLUOstar plate reader. The interactions across the dose matrix were determined by the Loewe Additivity and Bliss independence models using Horizon's Chalice Combination Analysis Software.

For generating compound resistance in vitro by dose escalation, A375 parental cells (ATCC CRL-1619) were grown to ~40-60% confluence in Dulbecco's Modified Eagle's Medium (DMEM) supplemented with 10% heat-inactivated FBS and penicillin/streptomycin, then treated with initial doses of BVD-523, trametinib, or dabrafenib either alone or in combination at or slightly below each compound's IC₅₀; for combination studies, initial dosing was half of each compound's IC₅₀. Cells were allowed to grow until ~70-90% confluence and split; medium was refreshed every 3-4 days. When cells again reached ~40-60% confluence, the dose was escalated by the same increment (equivalent to the starting concentration) then moved to 1.5-fold increases in concentration followed by a further move to 2-fold increases if the cells continued to adapt rapidly (e.g., the first six doses of the dabrafenib escalation were: 5, 10, 15, 20, 25, and 37.5 nM). This process was repeated as required.

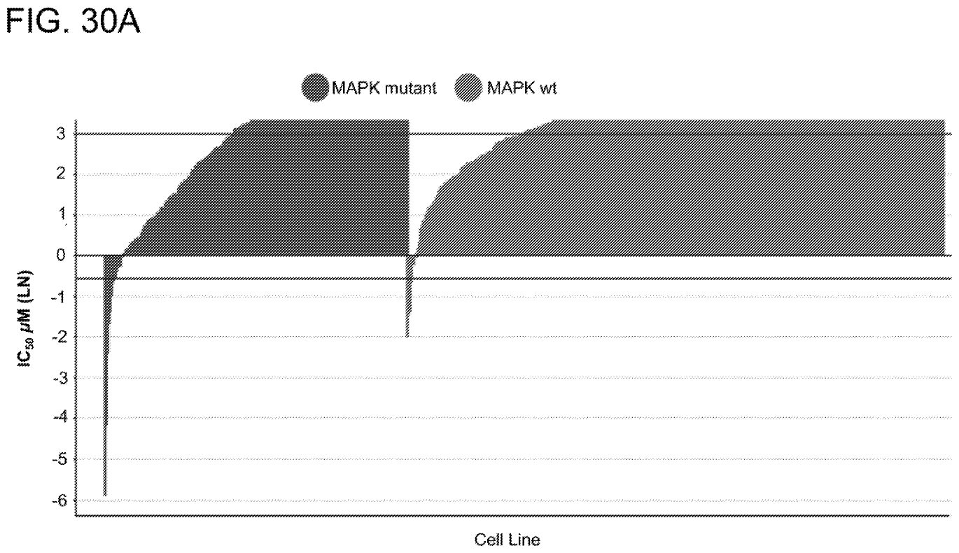

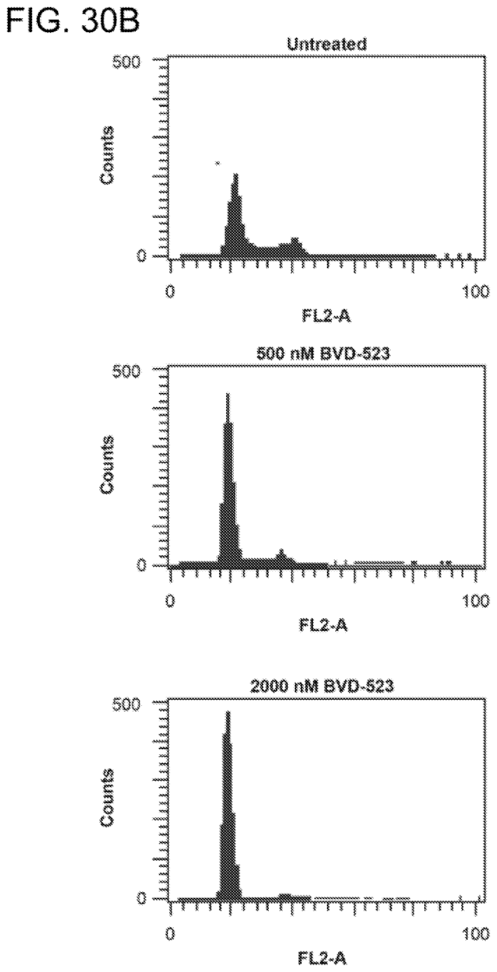

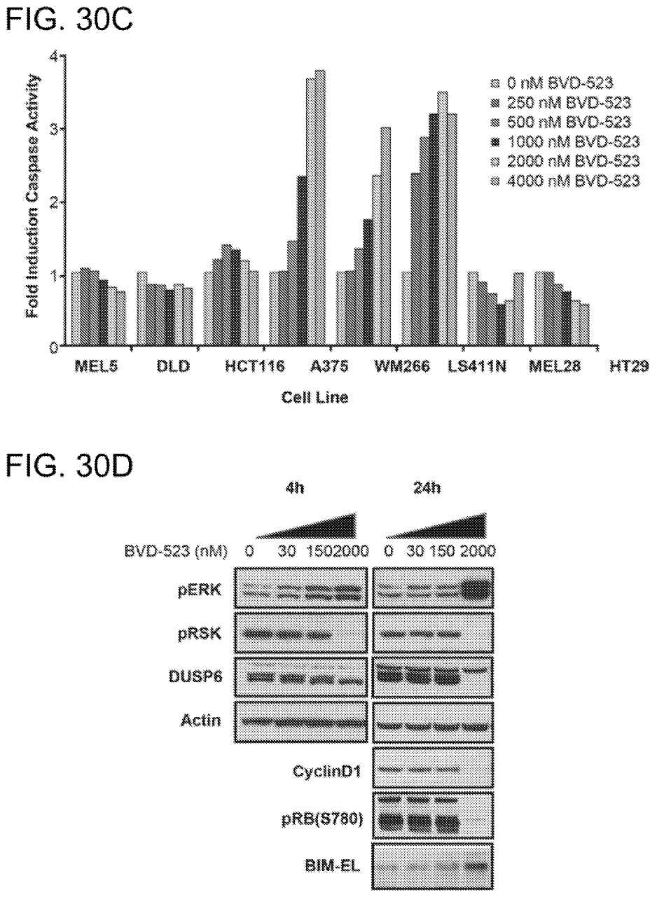

Cell viability assays for FIG. 30A were performed by a Resazurin (Alamar Blue) metabolic assay after 5 days in drug in full serum under high glucose conditions. Cells were seeded in 384-well microplates at ~15%-50% confluence in medium with 10% FBS and penicillin/streptavidin plus high glucose (18-25 mM). The optimal cell number for each cell line was determined to optimize growth during drugging. For adherent cell lines, after overnight incubation cells were treated with 9 concentrations of each compound (2-fold dilutions series) using liquid handling robotics, and returned to the incubator for assay at a 96-h time point. For suspension cell lines, cells were treated with compound immediately after plating and returned to the incubator for a 96-h time point. Cells were then stained with 55 μg/ml Resazurin (Sigma) prepared in glutathione-free media for 4 hours. Quantitation of fluorescent signal intensity was performed using a fluorescent plate reader at excitation and emission wavelengths of 535/595 nm for Resazurin. All screening plates were subjected to stringent quality control measures. Effects on cell viability were measured and a curve-fitting algorithm was applied to the raw dataset to derive a multi-parameter description of drug response, including the half maximal inhibitory concentration (IC₅₀). IC₅₀ is expressed in natural log of the IC₅₀ in μM (LN_IC₅₀; EXP returns IC₅₀ in μM). Extrapolation of the IC₅₀ was allowed for where it yielded very high values. If desired the data was restricted to the tested concentration range by capping IC₅₀ values at the maximum tested concentration (and the minimum tested concentration for low values).

For efficacy testing of BVD-523 in a patient-derived xenograft (AT052C) representing melanoma from a BRAFV600E patient that had become clinically refractory to vemurafenib. Tumor fragments were harvested from host animals and implanted into immune-deficient mice. The study was initiated at a mean tumor volume of approximately 170 mm³, at which point the animals were randomized into four groups including a control (1% [v/v] CMC p.o., BID×31) and three treatment groups (BVD-523 [100 mg/kg], dabrafenib [50 mg/kg], or BVD-523/dabrafenib [100/50 mg/kg], n=10/group); All treatment drugs were administered p.o. on a BID×31 schedule.

For IC₅₀ determination for the inhibition of PMA-stimulated RSK1 phosphorylation by BVD-523 in human whole blood samples, IC₅₀ values for the inhibition of PMA stimulated RSK1 phosphorylation by BVD-523 were determined for 10 healthy donors (aged 22-61 years) using an 8-point concentration curve ranging from 10 μM to 5 nM of BVD-523. Controls consisted of 3 unstimulated samples and 3 PMA-stimulated samples for each donor. Both phosphor-RSK (pRSK) and total RSK levels were determined and data were calculated using pRSK/RSK levels for each sample.

Thirty milliliters of blood was drawn from each donor into sodium heparin vacutainers. One mL of whole blood was added to each of twenty-two 2-mL microtubes per donor. The microtubes tubes were labeled with the donor number (1 through 10) and the subsequent treatment designation: “A” for PMA stimulation only (maximum), “B” for BVD-523-containing samples that received PMA stimulation; and “C” for the unstimulated samples (minimum). Dimethyl sulfoxide (DMSO) was added to all tubes in groups A and C to a final concentration of 0.1%. Samples were then rocked gently at room temperature.

BVD-523 (10 mM in 100% DMSO) was serially diluted with 3-fold dilutions into 100% DMSO. These serially diluted BVD-523 samples in 100% DMSO were then diluted 10-fold in Dulbecco's Modified Eagle Medium containing 10% fetal bovine serum and penicillin/streptomycin/glutamine, and 10 μL of each of these working solutions was added per mL of blood for each designated BVD-523 concentration. Each concentration of BVD-523 was run in duplicate, two 1-mL blood samples each, yielding 16 total samples for the full 8-point concentration curve. Samples were then rocked gently at room temperature for a minimum of 2 hours but not longer than 3 hours.

Human whole blood samples in groups A and B for all donors were stimulated with PMA at a final concentration of 100 nM for 20 minutes at room temperature. Samples in group C were not treated with PMA but were rocked and handled as all other samples.

Upon completion of PMA treatment for each sample, peripheral blood mononuclear cells were isolated from the human whole blood. One mL of blood from each sample was gently layered onto 0.75 mL of room-temperature Histopaque 1077 in a 2-mL microcentrifuge tube. The samples were centrifuged for 2 minutes at 16,000×g in an Eppendorf microcentrifuge. The interface and upper layers were removed and added to tubes containing 1 mL of cold Dulbecco's phosphate-buffered saline (DPBS). These samples were then centrifuged for 30 seconds at 16,000×g to pellet the cells. The buffer supernatant was removed by aspiration and the pellets were re-suspended in 1 mL of cold DPBS. The pellets from each sample were then re-pelleted as above. The buffer was removed by aspiration and the pellets were lysed as indicated below.

Complete lysis buffer consisted of Meso Scale Discovery Tris lysis buffer, 1× Halt Protease inhibitor cocktail, 1× Phosphatase inhibitor cocktail 2, 1× Phosphatase inhibitor cocktail 3, 2 mM phenylmethanesulfonyl fluoride, and 0.1% sodium dodecyl sulfate. Lysis buffer was kept on ice and made fresh for each sample group. Final cell pellets were lysed by the addition of 120 μL of complete lysis buffer. Samples were vortexed until the cell pellet disappeared and then flash frozen on dry ice. Samples were stored at −20° C. prior to measurement of pRSK and total RSK by ELISA.

For the pRSK ELISA (PathScan), thawed lysates were combined 1:1 with sample diluent (provided in ELISA kit): 120 μL of lysate added to 120 μL of sample diluent in a round bottom 96-well plate. This combination was then transferred to the pRSK microwells at 100 μL per well. For the total RSK ELISA (PathScan), 20 μL of the lysate already diluted 1:1 in sample diluent was further diluted in 200 μL of sample diluent in a round bottom 96-well plate. This combination was then transferred to the total RSK microwells at 100 μL per well. The plates were sealed with a plate seal and incubated 16 to 18 hours at 4° C., a time that was shown to yield the best detection of the target protein. Both ELISAs were developed according to the kit instructions.

Patients aged ≥18 years were eligible for participation if they had noncurable, histologically confirmed metastatic or advanced stage malignant tumors; an ECOG performance status of 0 or 1; adequate renal, hepatic, bone marrow, and cardiac function; and a life expectancy ≥3 months. Patients may have received up to 2 prior lines of chemotherapy for their metastatic disease. Exclusion criteria were known uncontrolled brain metastases; gastrointestinal conditions which could impair absorption of study medication; history or current evidence/risk of retinal vein occlusion or central serous retinopathy; and concurrent therapy with drugs known to be strong inhibitors of CYP1A2, CYP2D6, and CYP3A4 or strong inducers of CYP3A4. All participants provided informed consent prior to initiation of any study procedures.

Patients that received at least one dose of BVD-523 were included in the analysis using SAS (version 9.3) software. The data cutoff was Dec. 1, 2016. This study is registered with ClinicalTrials.gov, number NCT01781429.

The present invention presents data from an open-label, multicenter phase I study to assess the safety, pharmacokinetics, and pharmacodynamics of escalating doses of BVD-523 in patients with advanced malignancies. The dosing regimen combined both accelerated titration and standard cohort 3+3 dose escalation schema, which were used jointly to identify the MTD and RP2D of BVD-523 in patients with advanced solid tumors. One to 6 patients per treatment cohort were assigned to receive sequentially higher oral doses of BVD-523 on a BID schedule (12-hour intervals) in 21-day cycles, starting at a dose of 10 mg BID. BVD-523 was administered BID continuously in 21-day cycles at the following doses: 10 mg (n=1); 20 mg (n=1); 40 mg (n=1); 75 mg (n=1); 150 mg (n=1); 300 mg (n=4); 600 mg (n=7); 750 mg (n=4); and 900 mg (n=7).

Patients received BID oral doses until disease progression, unacceptable toxicity, or a clinical observation satisfying another withdrawal criterion. Dose escalations occurred in up to 100% increments in single-patient cohorts until 1 patient experienced a ≥Grade 2 toxicity (excluding alopecia or diarrhea). Cohorts were then expanded to at least 3 patients each and subsequent dose-escalation increments were reduced from up to 100% to a maximum of 50%. When at least 1 patient in a 3-patient cohort experienced a DLT, up to 3 additional patients were treated at this dose level. When more than 1 DLT occurred in $6 patients, this dose level was defined as the nontolerated dose and dose escalation was stopped. Intrapatient dose escalation was allowed, provided the patients receiving the highest current dose had been observed for at least 3 weeks and dose-limiting side effects were reported in fewer than 2 of 6 patients assigned to a given dose. Patients experiencing DLTs or unacceptable toxicity had their treatment interrupted until the toxicity returned to ≤Grade 1. Resumption of BVD-523 treatment was then initiated at the next lower dose level tested or at a 20% to 30% dose decrease, aligning with capsule dosage.

The primary objective of the phase I study was to define the safety and tolerability of BVD-523 by determining the dose-limiting toxicities, the MTD, and the RP2D. The secondary objectives included the determination of the pharmacokinetic profile of BVD-523 in patients with advanced malignancies and the investigation of any preliminary clinical effects on tumor response, as assessed by physical or radiologic exam using RECIST v1.1. The exploratory objectives included evaluation of pharmacodynamic marker (biomarker) measures and investigation of preliminary clinical effects on tumor response assessed by ¹⁸F-FDG-PET as indicated.

For determination of MTD, DLT, and RP2D, MTD was defined as the highest dose cohort at which ≤33% of patients experienced BVD-523-related DLTs in the first 21 days of treatment. DLT was defined as a BVD-related toxicity in the first 21 days of treatment that resulted in ≥Grade 4 hematologic toxicity for >1 day; Grade 3 hematologic toxicity with complications (e.g., thrombocytopenia with bleeding); ≥Grade 3 nonhematologic toxicity, except untreated nausea, vomiting, constipation, pain, and rash (these become DLTs if the AE persisted despite adequate treatment); or a treatment interruption exceeding 3 days in Cycle 1 (or the inability to being in Cycle 2 for >7 days) due to BVD-523-related toxicity.

The RP2D could be as high as the MTD and was determined in discussion with the clinical investigators, the medical monitor, and the sponsor. Observations related to pharmacokinetics, pharmacodynamics, and any cumulative toxicity observed after multiple cycles were included in the rationale supporting the RP2D.

With regard to safety assessments, AEs were defined as any untoward medical occurrence in a patient who was administered a medicinal product that does not necessarily have a causal relationship with BVD-523, and was coded using the MedDRA coding dictionary. An SAE was any untoward medical occurrence that occurred at any dose that resulted in death, was life-threatening, required inpatient hospitalization or prolongation of existing hospitalization, or resulted in persistent or significant disability/incapacity or a congenital anomaly/birth defect. The severity of AEs were graded according to the National Cancer Institute Common Terminology Criteria for Adverse Events, Grading Scale, version 4.

Safety evaluations were conducted at baseline, on Days 8, 15, 22, 29, 36, and 43, and, in patients who continued treatment, every 3 weeks or if clinically indicated thereafter. Each evaluation included a physical examination and clinical laboratory studies. Electrocardiograms were repeated if clinically significant and at the discretion of the investigator. The investigators made judgments regarding whether or not AEs were related to study drug and followed up until resolution or stabilization, or the AE was judged to be no longer clinically significant.

For pharmacokinetic analysis, the pharmacokinetic population consisted of patients who received at least one dose of BVD-523 and had evaluable pharmacokinetic data for plasma and/or urine. Blood samples were collected prior to dosing, and then at 0.5 (±5 min), 1 (±5 min), 2 (±10 min), 4 (±10 min), 6 (±10 min), 8 (±10 min), and 12 (±2 hr) hours on Day 1 (Visit 2; baseline/initiation of treatment) and Day 15 (Visit 4; at steady state) after the morning dose. On Day 22, prior to dose administration, a final blood sample was collected for pharmacokinetic analyses. Urine samples were collected predose and at the 1- to 6-hour and 6- to 12-±2-hour intervals postdose on Days 1 and 15. Plasma and urine samples were analyzed for BVD-523 and metabolites using validated LC/MS/MS methods. Standard pharmacokinetic parameters were obtained using Phoenix WinNonlin (Pharsight) with a noncompartmental method. Relationship between dose and exposure was calculated using standard least-squares regression analysis.

For pharmacodynamic confirmation of target inhibition by BVD-523, targeted ERK inhibition by BVD-523 was determined by examining pRSK as a target biomarker in human whole blood samples obtained from patients with advanced solid tumors (N=27) who had received different doses of BVD-523 (10-900 mg BID) during the phase I study. The activity of BVD-523 from 4 timepoints (baseline predose, baseline 4 hours postdose, Day 15 predose, and Day 15 4 hours postdose) was expressed as a percent activity (pRSK) of PMA-stimulated blood incubated with BVD-523.

For measurement of antitumor response, tumor measurements based on physical examination occurred at baseline and on the first day of each treatment cycle. CT and other assessments were made every 2 to 3 cycles. Findings were assessed in accordance with RECIST v1.1: CR was defined as disappearance of all target lesions; PR was defined as a ≥30% decrease in the sum of the longest diameters of target lesions, taking baseline measurements as reference; stable disease was defined as being of neither sufficient shrinkage to qualify for PR nor sufficient increase to qualify for progressive disease, taking as reference the baseline measurement. Metabolic response was assessed by visualizing tumor uptake of ¹⁸F-glucose via ¹⁸F-FDG-PET scanning prior to receiving the first dose of BVD-523 and at Day 15 (Visit 4).

### Example 2

### Dose Escalation and Proliferation Assays—Month 1

### Dose Escalation Progress—Month 1

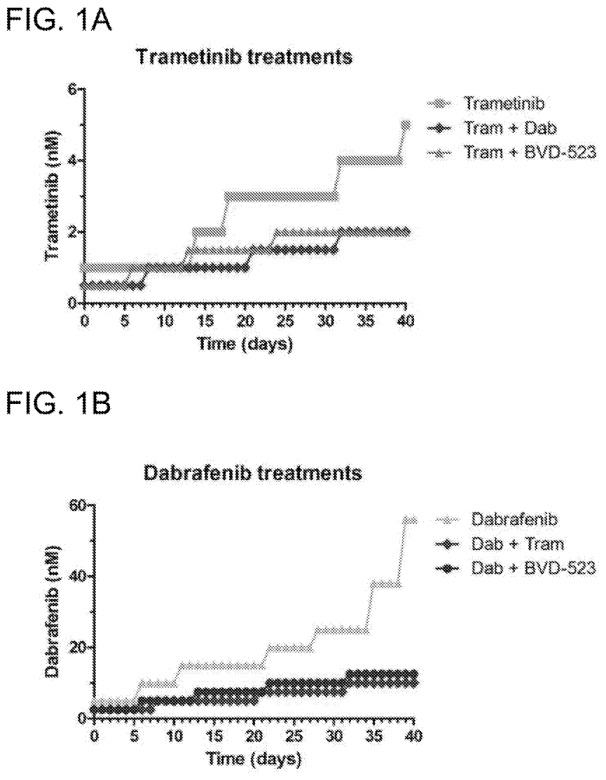

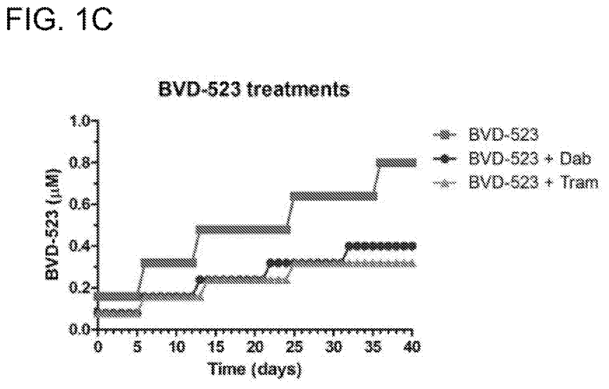

A375 cells were dose escalated using BVD-523, dabrafenib, and trametinib either as single agents or in combination. Doses were increased in small increments during the first month. Other than a marked reduction in growth rate, cells generally tolerated the escalations well and the doses were planned to be more aggressively escalated using larger increments in month 2. FIG. 1A-FIG. 1C show month 1 progress for the dose escalation studies.

### Proliferation Assay Results—Month 1

Proliferation assays were performed to assess the response of the escalated cells lines vs. parental cell line, to BVD-523, dabrafenib, and trametinib treatments.

FIG. 2A-FIG. 2H show normalized and raw proliferation assay results from month 1 of the studies. Note that differences in max signals in DMSO controls between different treatments (FIG. 2D, FIG. 2F, and FIG. 2H) suggest differential growth rates between treatments. These differences may influence the responses of lines to inhibitors in the proliferation assays.
Table 10 shows IC₅₀ data for month 1 of the studies.

TABLE 10
IC₅₀ Data - Month 1

| | Cell Line, Relative IC₅₀ (nM) | | | | | | |
|---|---|---|---|---|---|---|---|
| | | | | BVD- | Dab/ | Dab/ | Tram/ |
| Compound | Par* | Tram | Dab | 523 | Tram | 523 | 523 |
| Dabrafenib | 6 | 29 | about | 8 | 58 | 68 | 11 |
| | | | 161 | | | | |
| Trametinib | 0.5 | 2.2 | 2.5 | 0.7 | 3.9 | 3.1 | 2.5 |
| BVD-523 | 189 | 335 | 350 | 268 | 300 | 412 | 263 |
| Paclitaxel | 2.2 | 3.0 | 3.3 | 3.4 | 3.5 | 3.4 | 3.4 |

*Par = Parental cell line

There were early hints that cells grown in the presence of escalating doses of dabrafenib or trametinib, either as single agents or in combinations, were exhibiting decreased responses to these two agents in proliferation assays.

In the early stages of month 2, the growth rate of cells in the dabrafenib only treatment notably increased relative to the early stages of month 1. This enabled an increased rate of progression and suggested that resistance was becoming apparent.

### Example 3

### Dose Escalation and Proliferation Assays—Month 2

### Dose Escalation Progress—Month 2

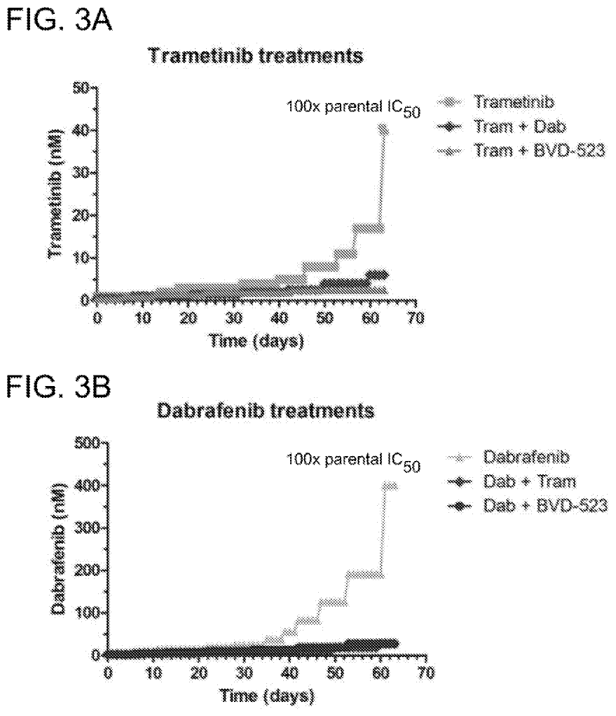

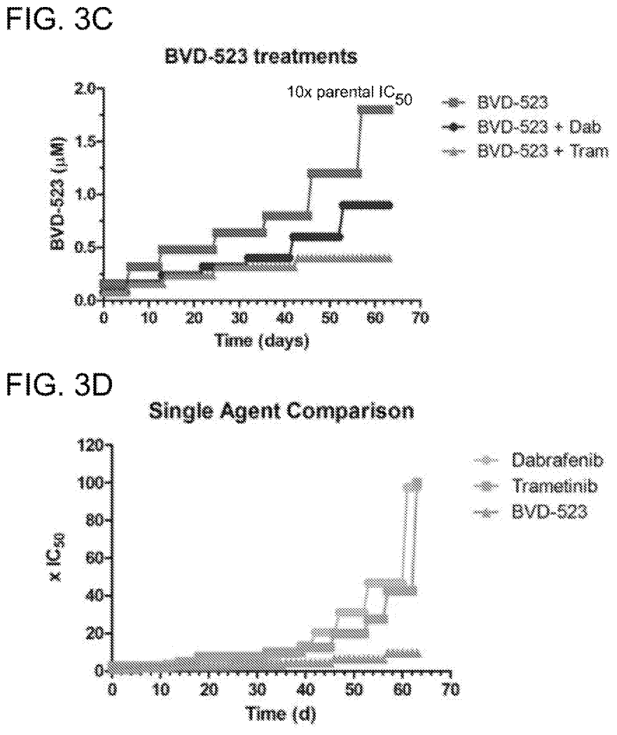

The second month of studies saw most treatments move into a phase where doses were increased in greater increments (1.5-fold) compared to the initial gentle escalation phase. The single agent escalation of dabrafenib and trametinib was quickest, with cells growing in concentrations equivalent to 100× parental cell IC₅₀ (FIG. 3A and FIG. 3B). The single agent escalation of BVD-523 progressed more slowly compared to dabrafenib and trametinib (FIG. 3C). See FIG. 3D for a comparison of the single agent escalations. BVD-523 escalated cells had a more “fragile” appearance and there was a greater number of floating cells compared to the dabrafenib and trametinib escalated populations.

The combined agent escalations progressed more slowly than the single agent treatments. The BVD-523/trametinib combination was particularly effective in preventing cells from progressing.

### Proliferation Assay Results—Month 2

Proliferation assays on single agent escalated dabrafenib and trametinib cell populations revealed modest shifts in the dose response curves, suggesting that an additional period of escalation would be beneficial to further enrich for resistant cells. Interestingly, in the proliferations assay, there was evidence to suggest that cells exposed to BVD-523 grew less well upon inhibitor withdrawal, perhaps indicating a level of addiction.

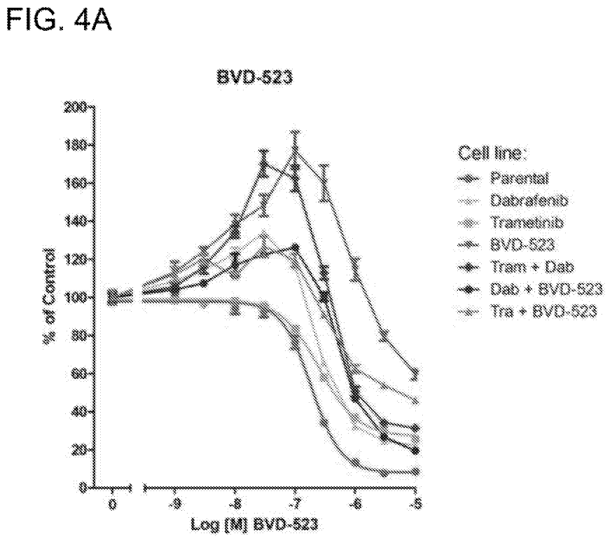

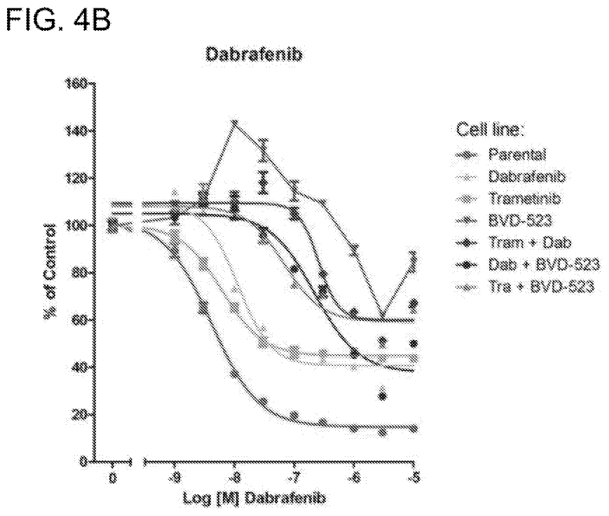

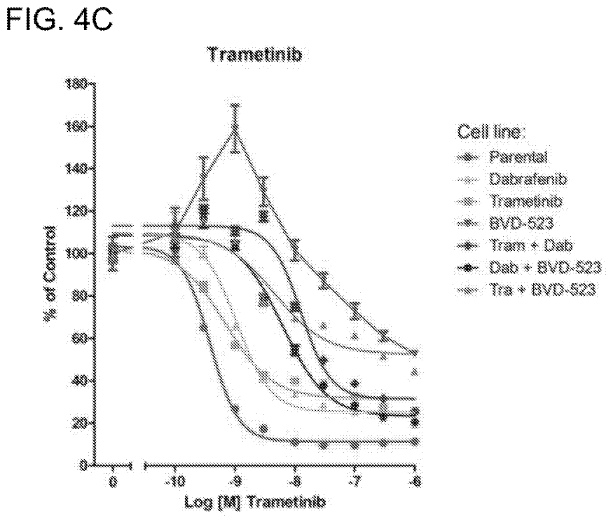

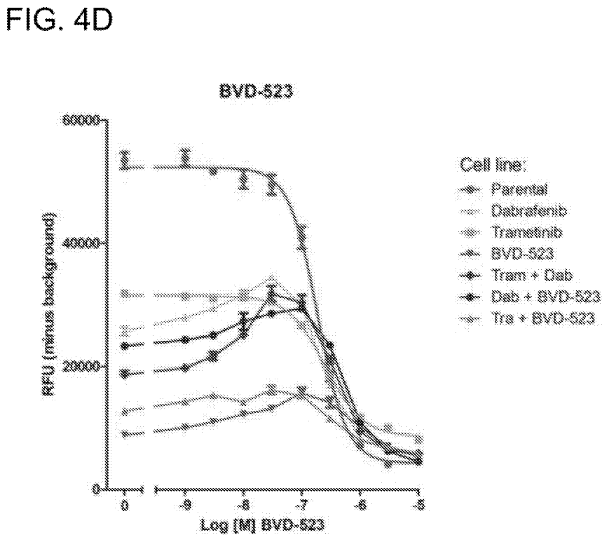

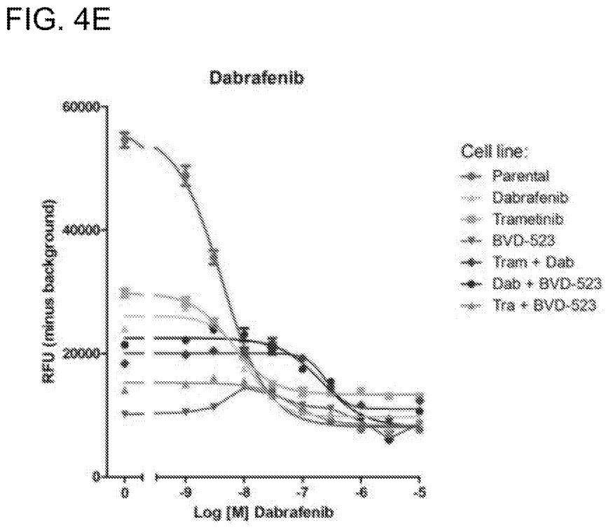

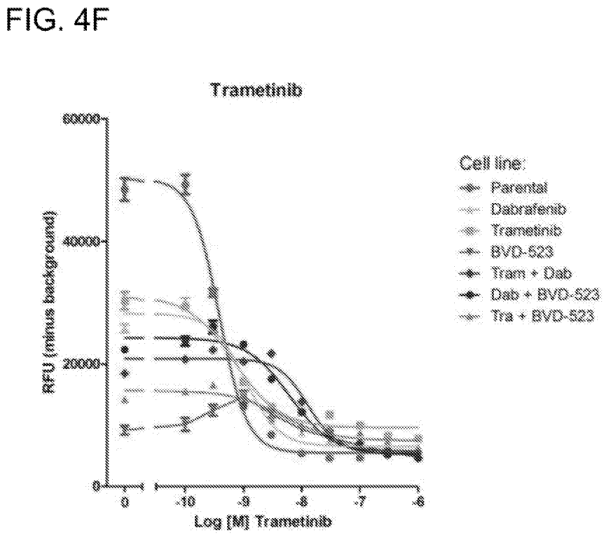

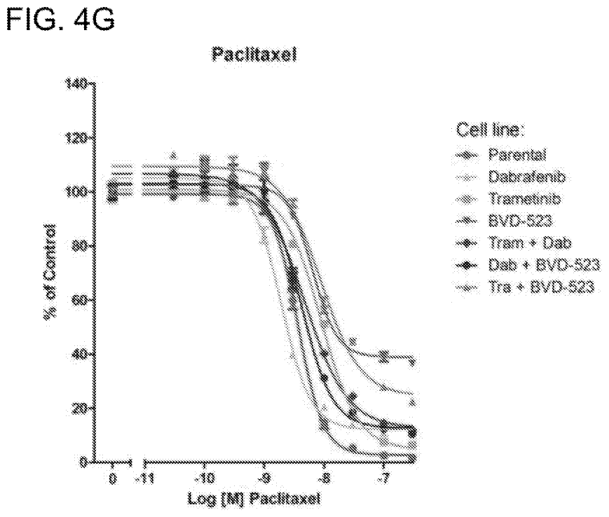

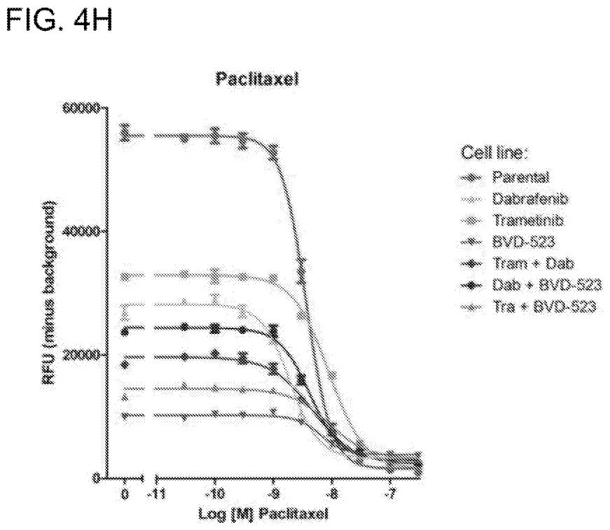

FIG. 4A-FIG. 4H show normalized and raw proliferation assay results from month 2 of the studies. Note that differences in max signals in DMSO controls between different treatments (FIG. 4D, FIG. 4F, and FIG. 4H) suggest differential growth rates between treatments. These differences may influence the responses of lines to inhibitors in the proliferation assays.

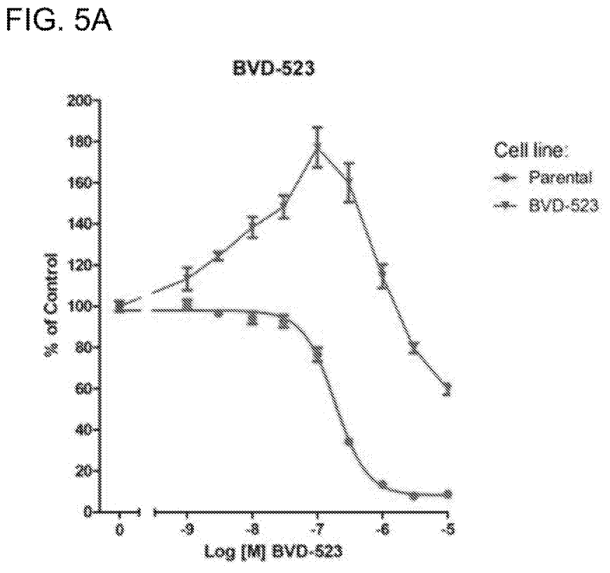

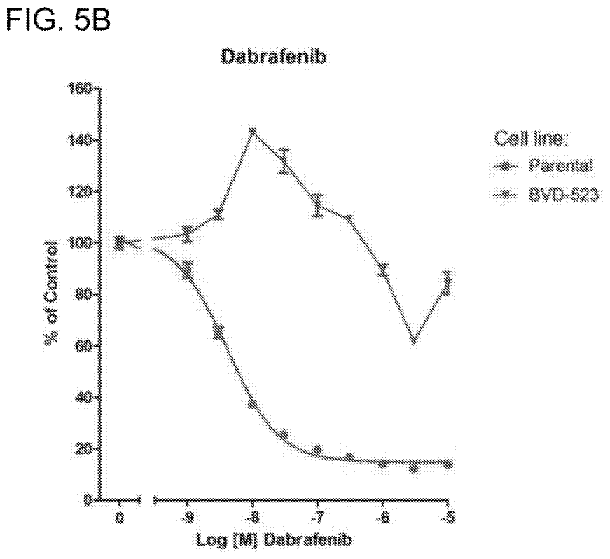

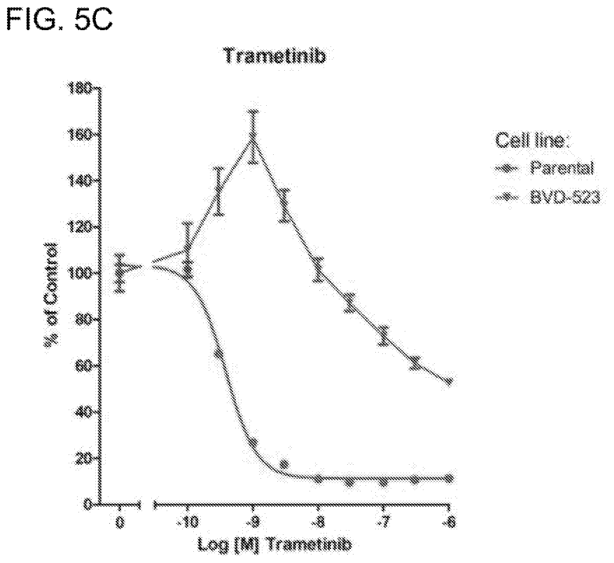

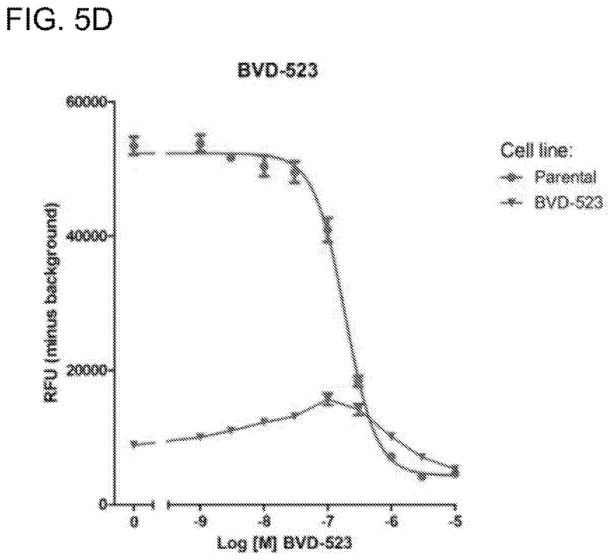

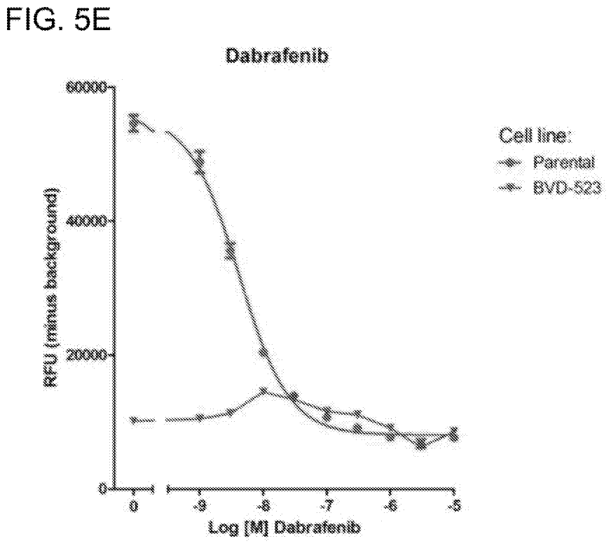

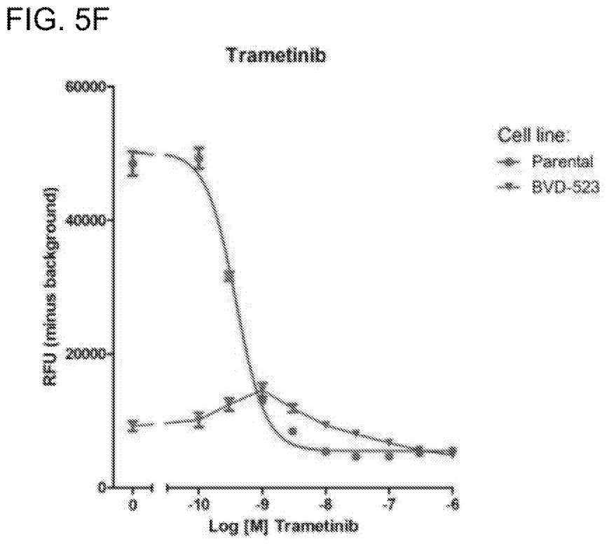

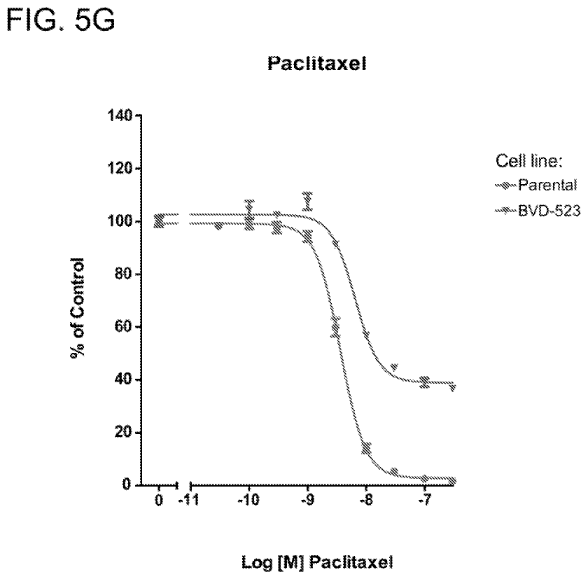

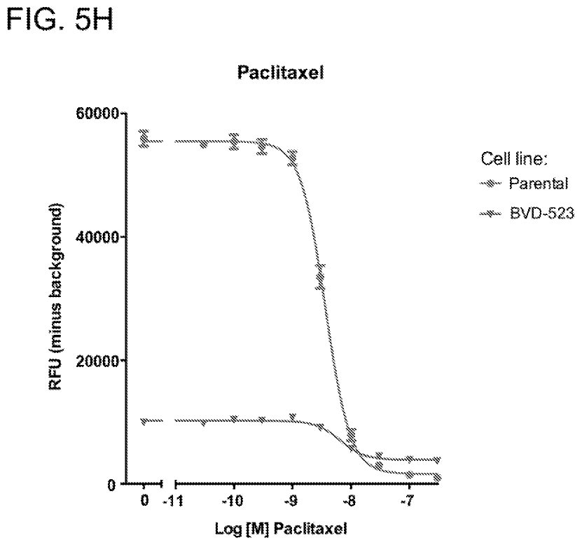

FIG. 5A-FIG. 5H show normalized and raw proliferation assay results from month 2 of the studies with a focus on parental and BVD-523 line data only.

Table 11 shows IC₅₀ data for month 2 of the studies. Relative IC₅₀s were determined from 4-parameter curve fits in Prism.

TABLE 11
IC₅₀ Data - Month 2

| | Cell Line, Relative IC₅₀ (nM) | | | | | | |
|---|---|---|---|---|---|---|---|
| | | | | BVD- | Dab/ | Dab/ | Tram/ |
| Compound | Par* | Tra | Dab | 523 | Tram | 523 | 523 |
| Dabrafenib | 4.1 | 6.2 | 11.5 | 697 | 256 | 218 | 68 |
| Trametinib | 0.4 | 0.7 | 1.1 | 24.3 | 12.6 | 6.2 | 4.6 |
| BVD-523 | 187 | 252 | 284 | 1706 | 561 | 678 | 435 |
| Paclitaxel | 3.7 | 8.9 | 1.9 | 6.5 | 4.7 | 4.2 | 8.9 |

*Par = Parental cell line

### Example 4

### Dose Escalation and Proliferation Assays—Month 3

### Dose Escalation Progress—Month 3

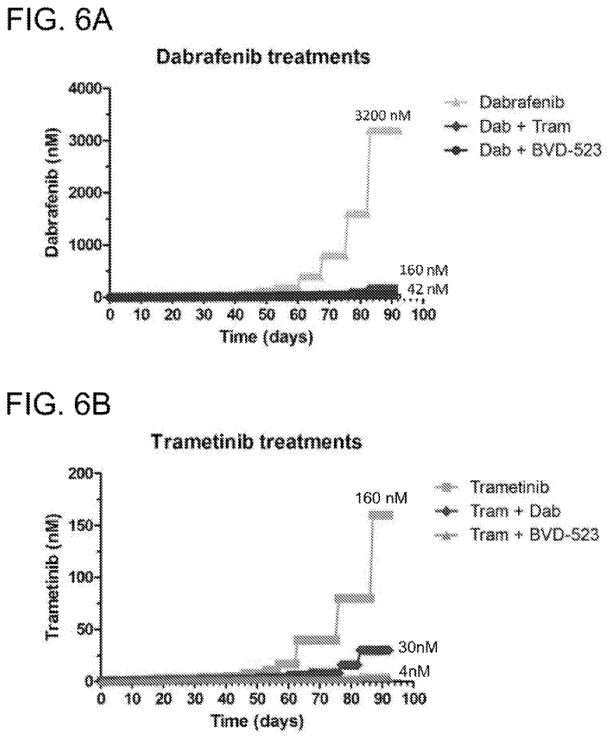

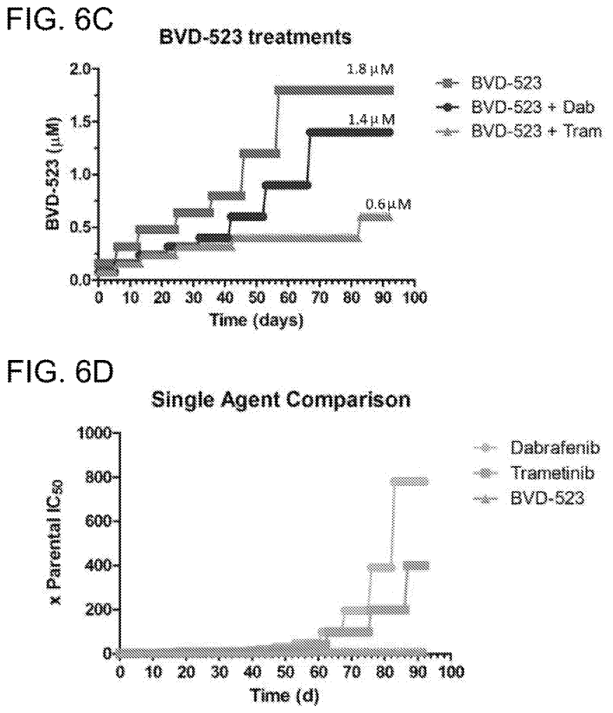

FIG. 6A-FIG. 6C show single and combination agent escalation for month 3 of the studies. FIG. 6D shows a comparison of single agent escalations.

### Proliferation Assay Results—Month 3

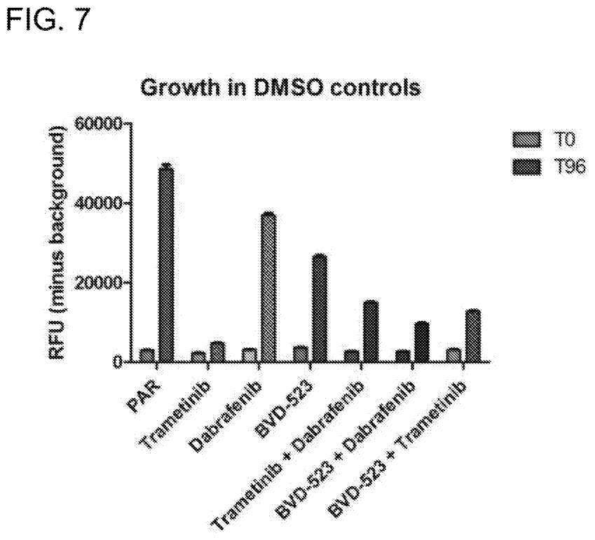

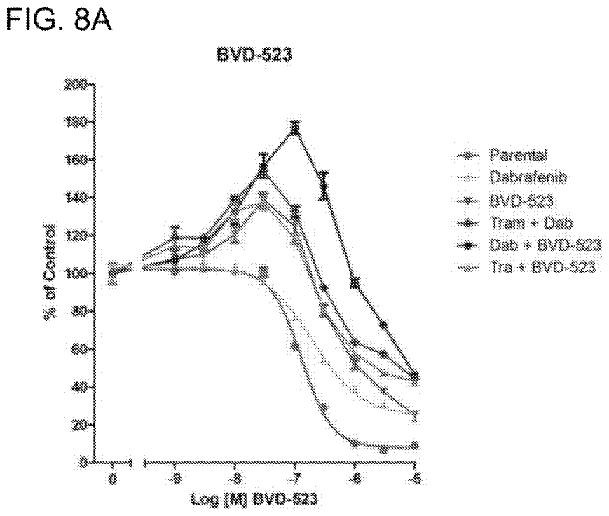

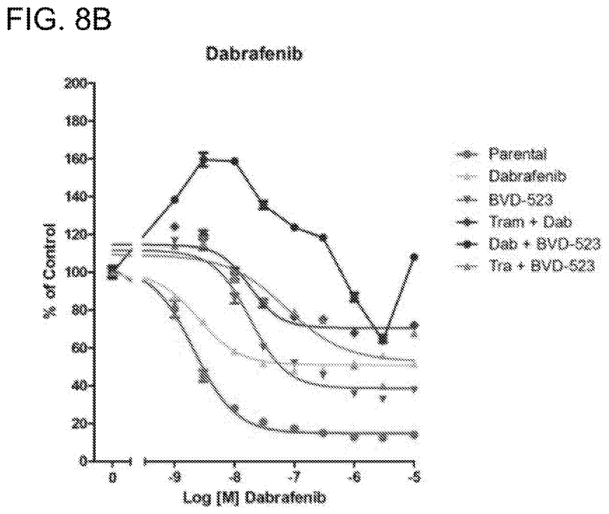

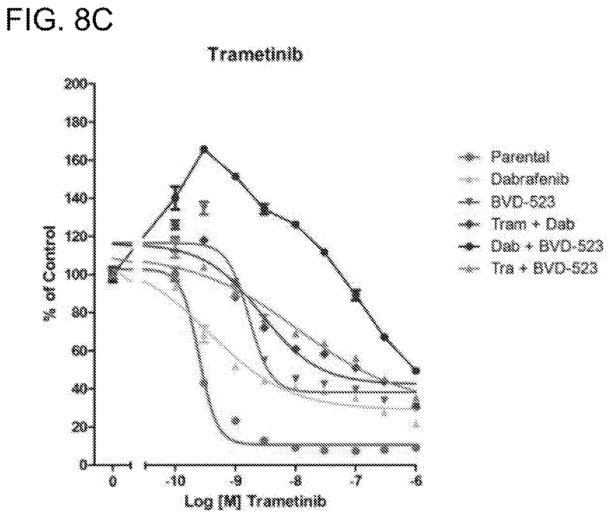

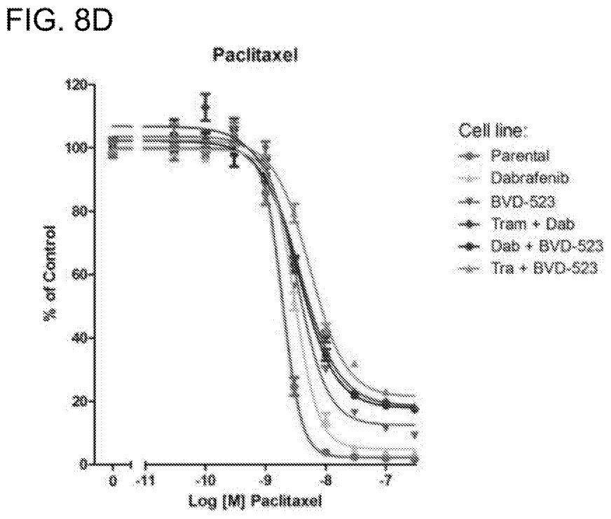

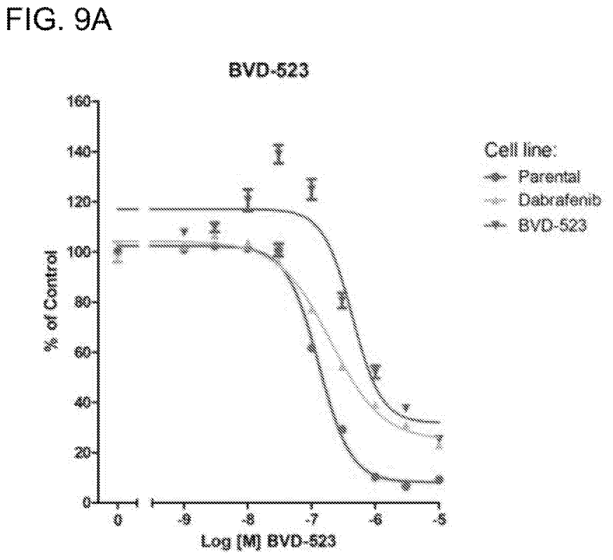

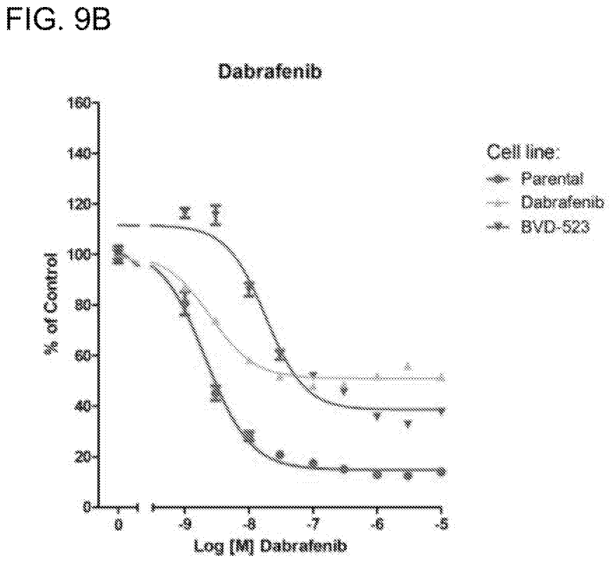

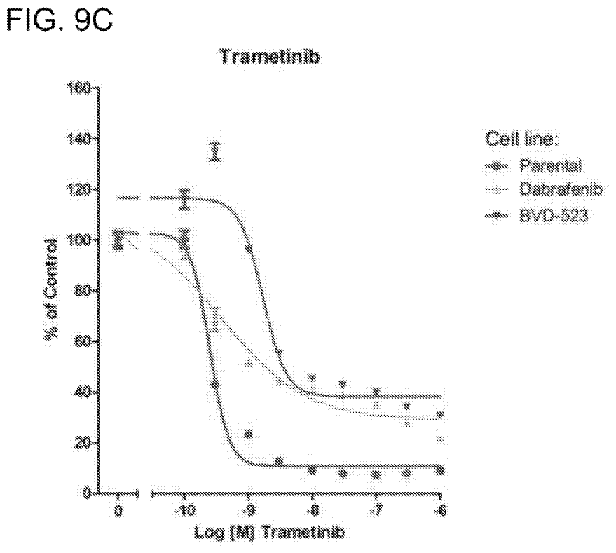

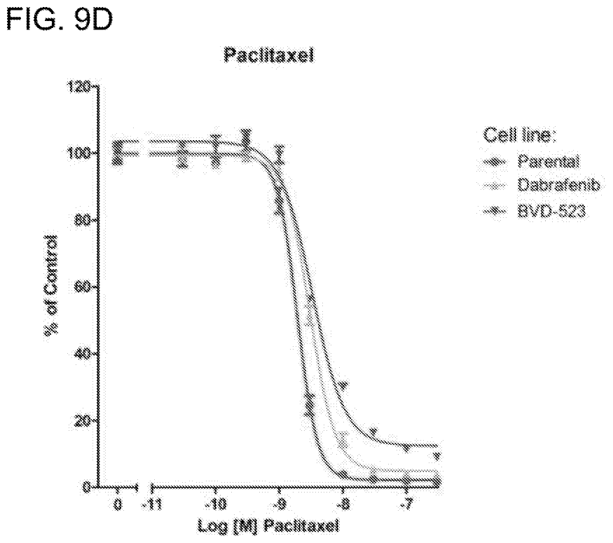

FIG. 7 shows an assessment of growth during the proliferation assay in DMSO control wells. FIG. 8A-FIG. 8D show results from month 3 of the studies. FIG. 9A-FIG. 9D show results from month 3 of the studies with a focus on single treatment cell lines.

Table 12 shows IC₅₀ data for month 3 of the studies. Relative IC₅₀s were determined from 4-parameter curve fits in Prism. IC₅₀ values were not determined for the cell line escalated with trametinib due to a lack of growth during the assay (ND: not done).

TABLE 12
IC₅₀ Data - Month 3

| | Cell Line, Relative IC₅₀ (nM) | | | | | | |
|---|---|---|---|---|---|---|---|
| | | | | BVD- | Dab/ | Dab/ | Tram/ |
| Compound | Par* | Tram | Dab | 523 | Tram | 523 | 523 |
| Dabrafenib | 2.1 | ND | 2.5 | 18.4 | 17.9 | 337 | 73 |
| Trametinib | 0.2 | ND | 0.4 | 1.7 | 2.7 | 90 | 11.2 |
| BVD-523 | 129 | ND | 198 | 433 | 323 | 1151 | 296 |
| Paclitaxel | 1.9 | ND | 1.9 | 6.5 | 4.7 | 4.2 | 8.9 |

*Par = Parental cell line

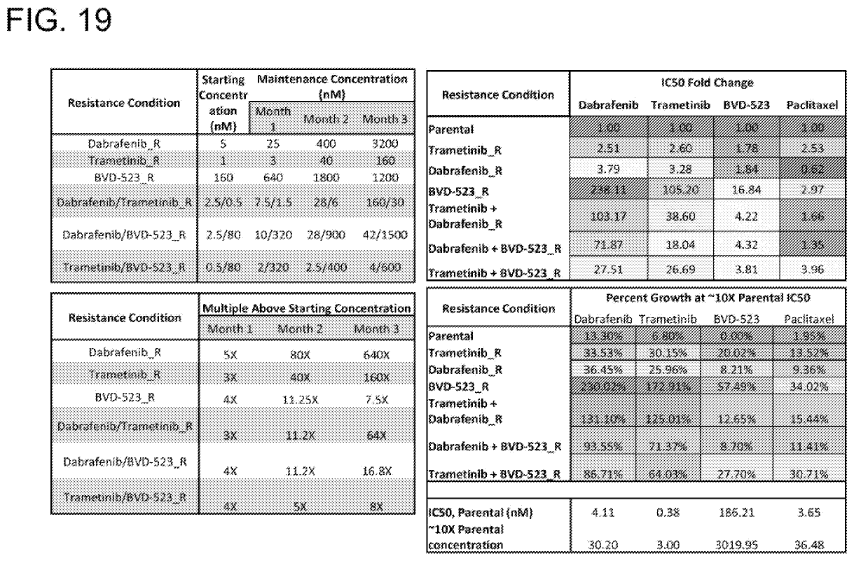

FIG. 19 shows single and combination agent escalation for month 3 of the studies. Cell line variants were obtained that could grow in the presence of dabrafenib or trametinib at concentrations greater than 100 times the IC₅₀ of these agents in parental A375 cell. In comparison, cell lines resistant to BVD-523 could only be maintained in less than 10× of parental IC₅₀ concentration. Sensitivity testing suggested dabrafenib and trametinib-resistant cell lines remained relatively sensitive to BVD-523; the increased IC₅₀ “shift” for BVD-523 in resistant cell lines was more modest than those corresponding IC₅₀ increases following dabrafenib or trametinib treatment. Likewise, compared to dabrafenib or trametinib treatment, more complete inhibition of cell growth was observed when resistant cell lines were treated with BVD-523 at concentrations 10-fold above its IC₅₀ in the parental A375 line. In total, patterns of resistance and cross-sensitivity suggest BVD-523 may remain effective in settings of acquired resistance.

### Example 5

### Combination Study Results

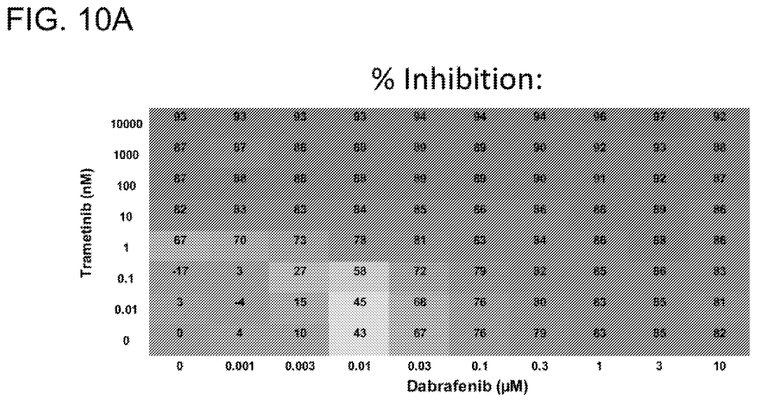

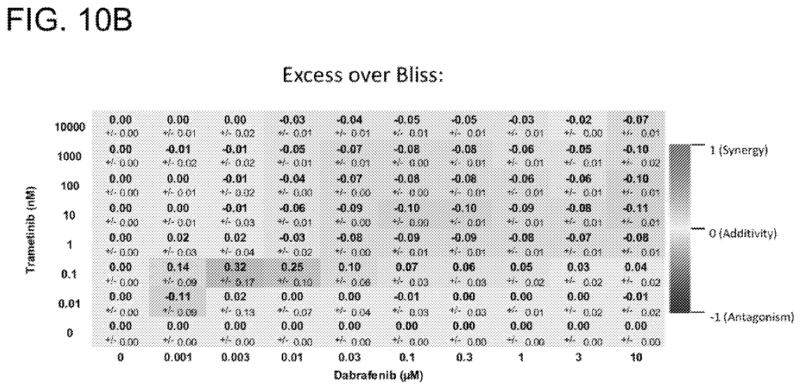

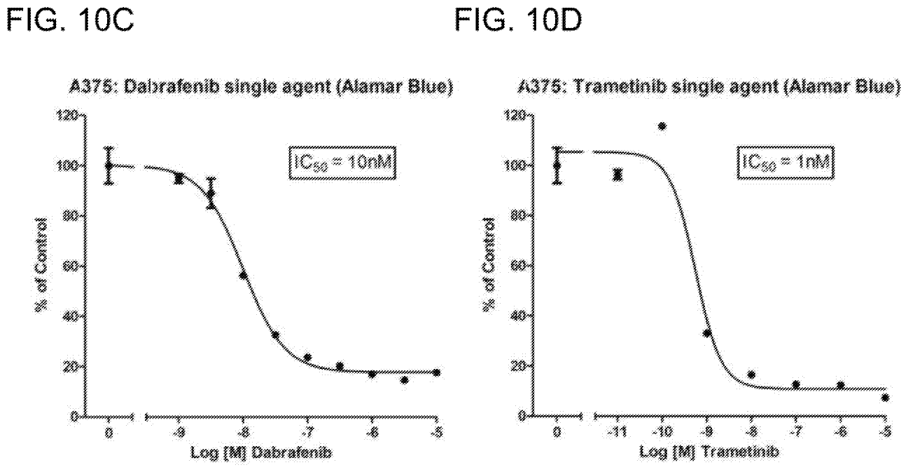

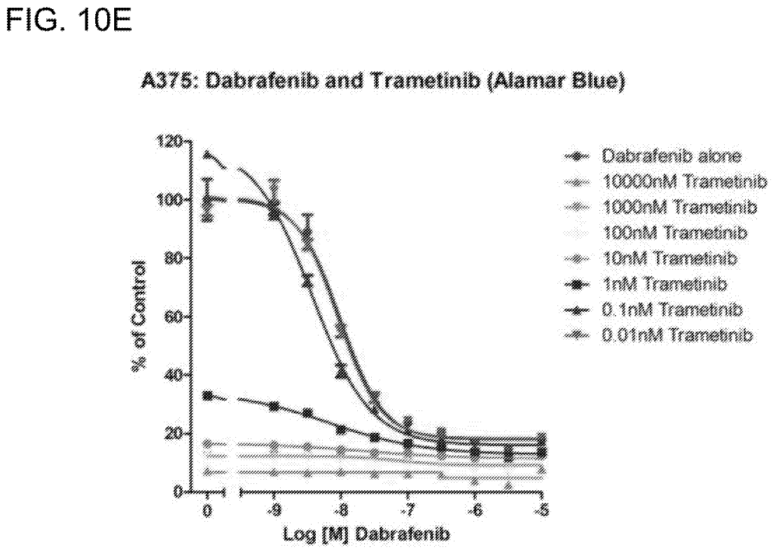

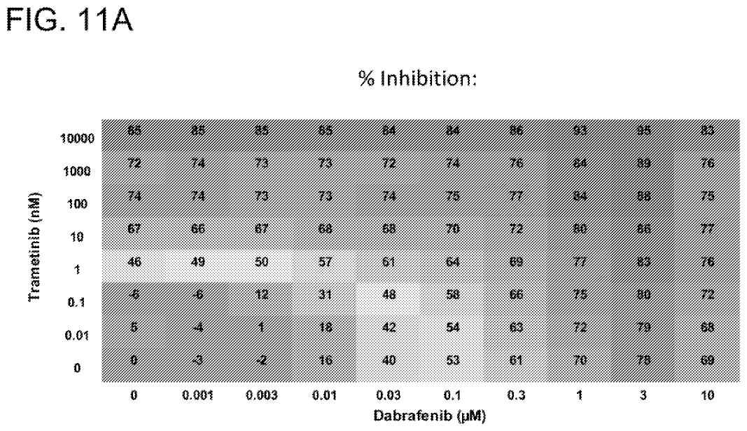

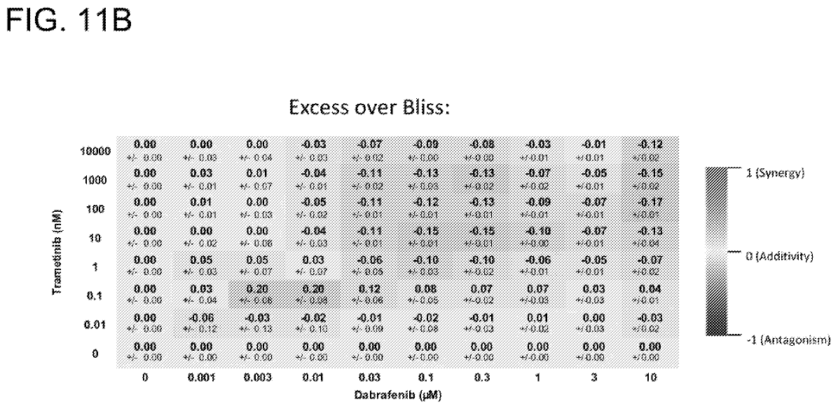

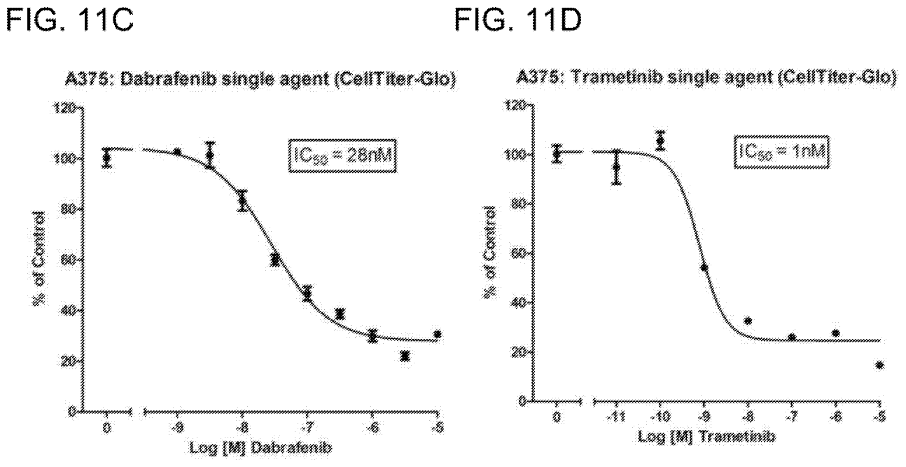

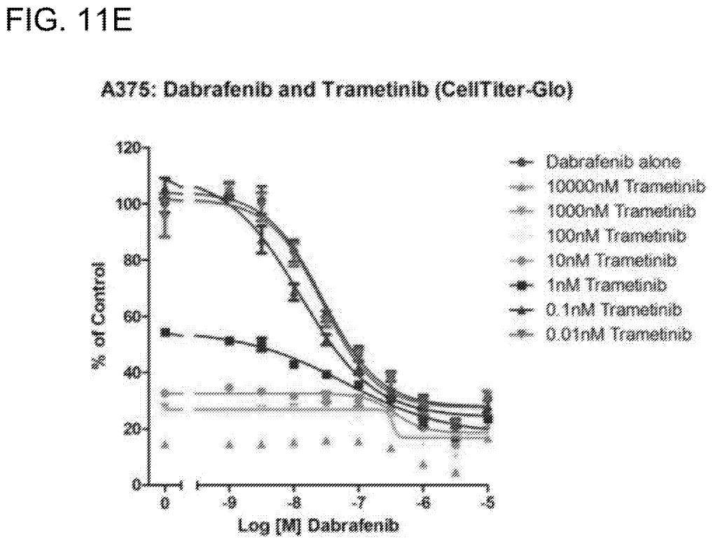

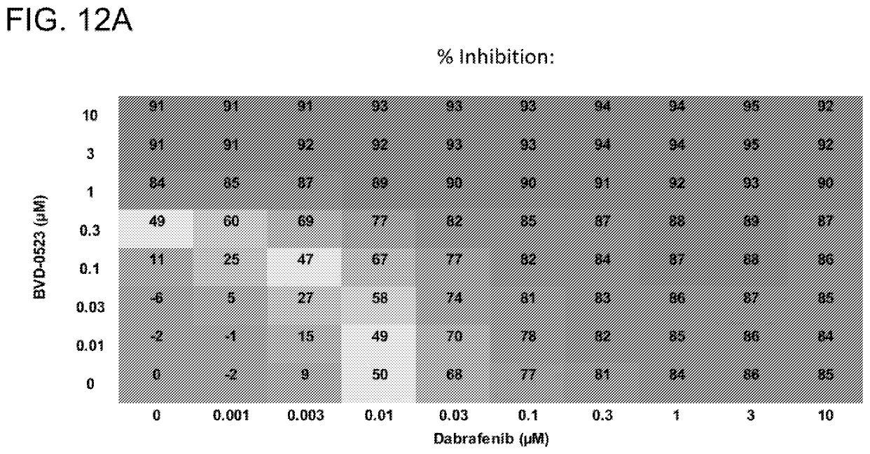

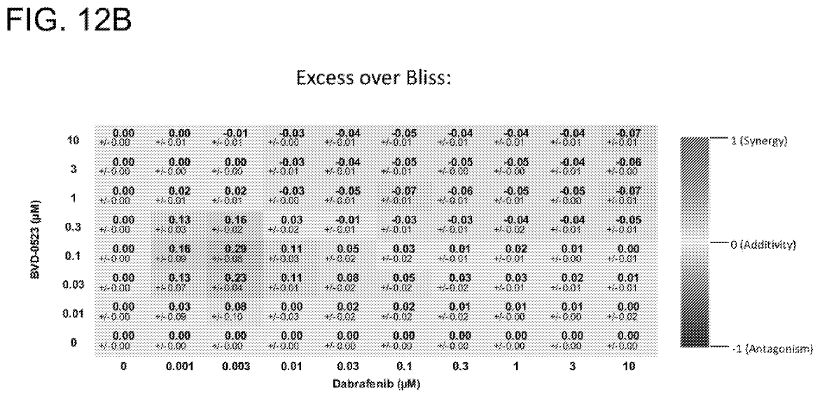

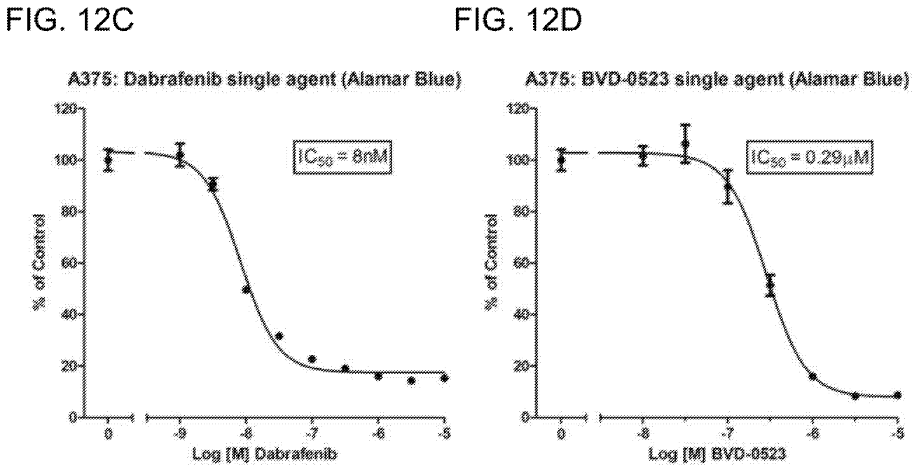

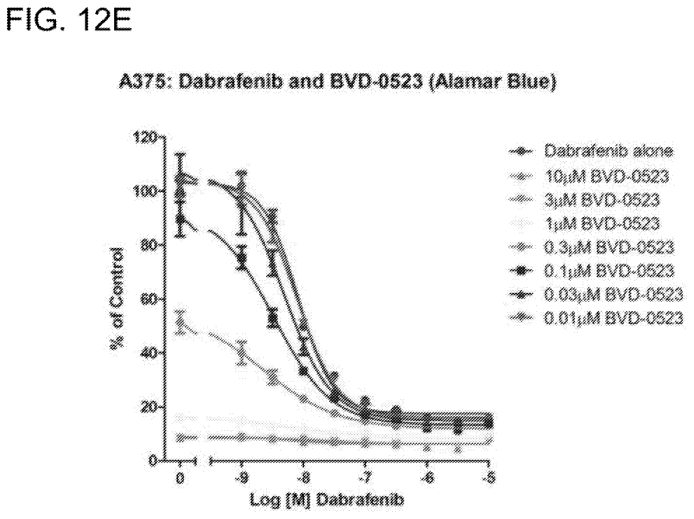

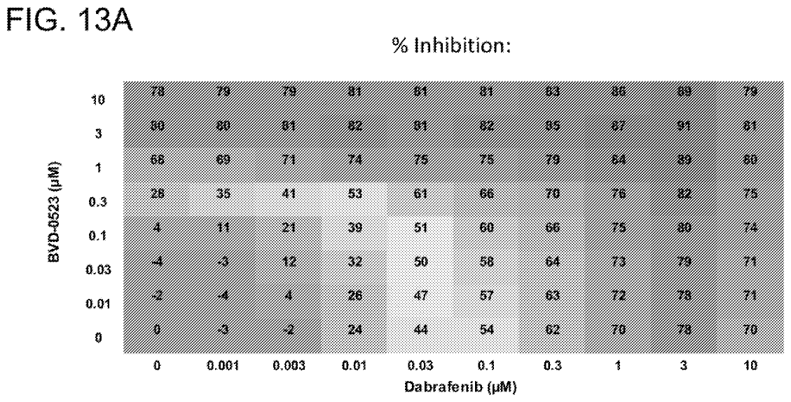

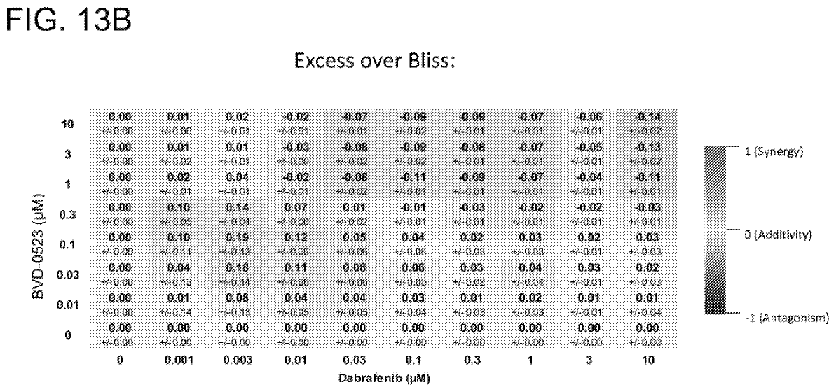

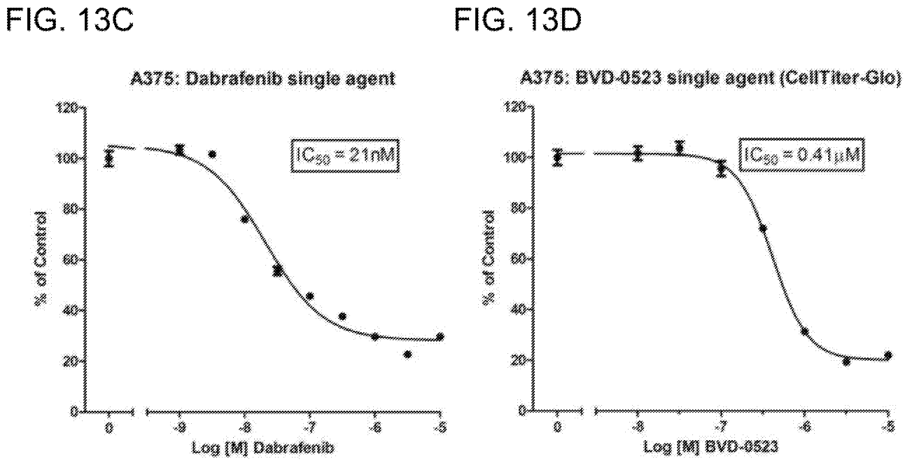

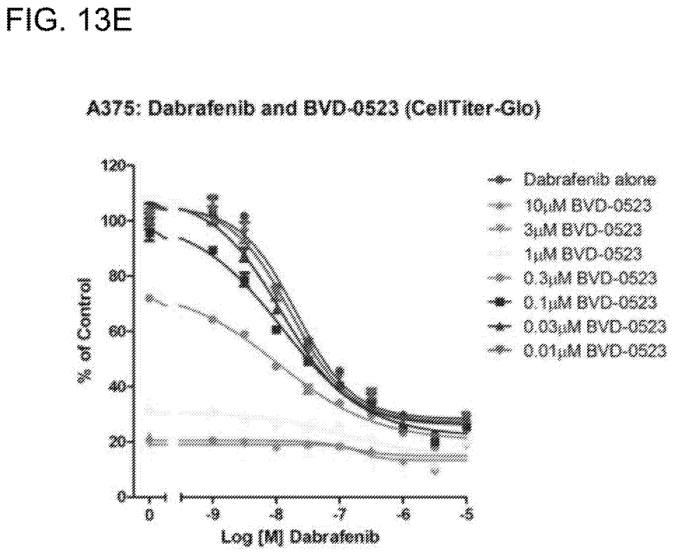

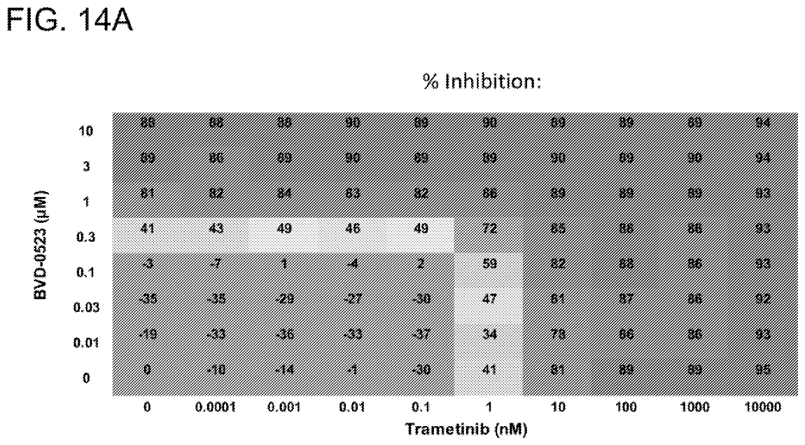

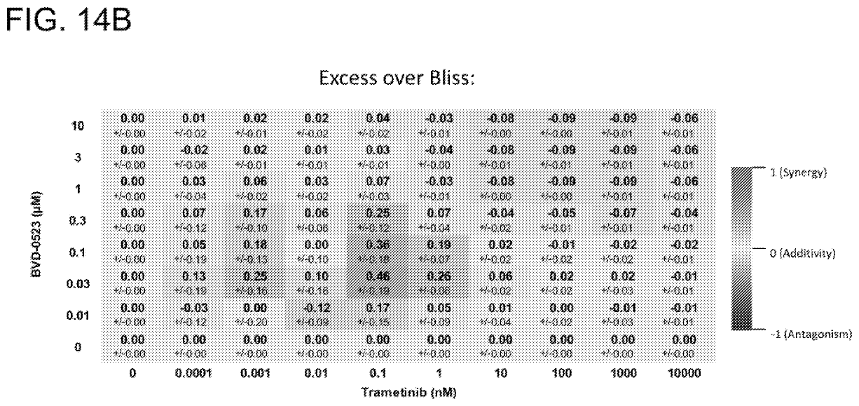

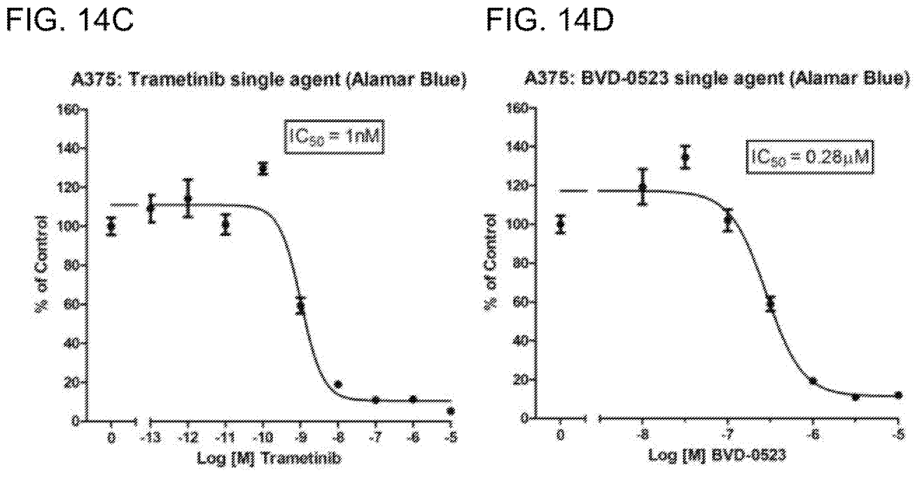

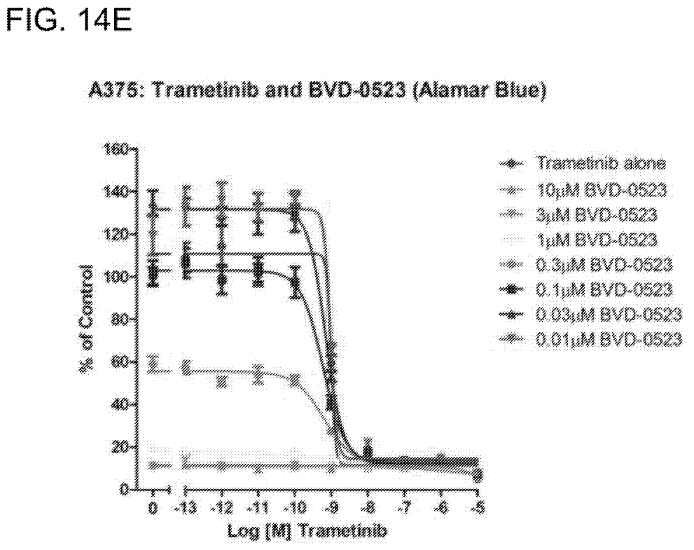

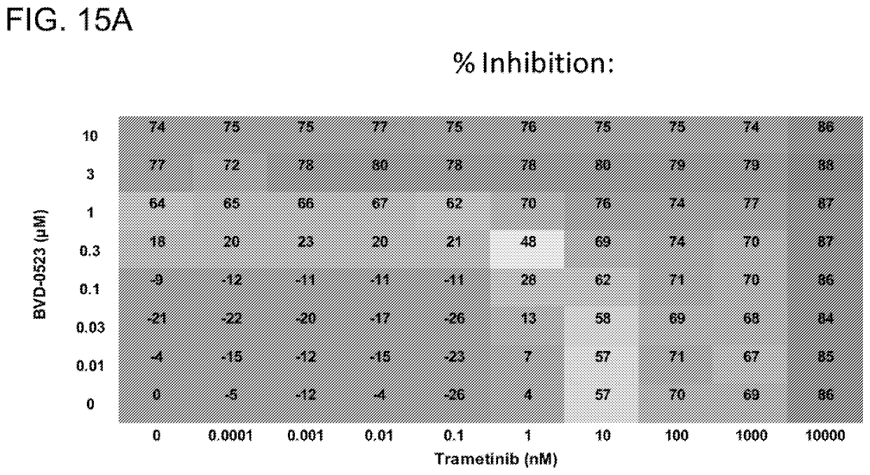

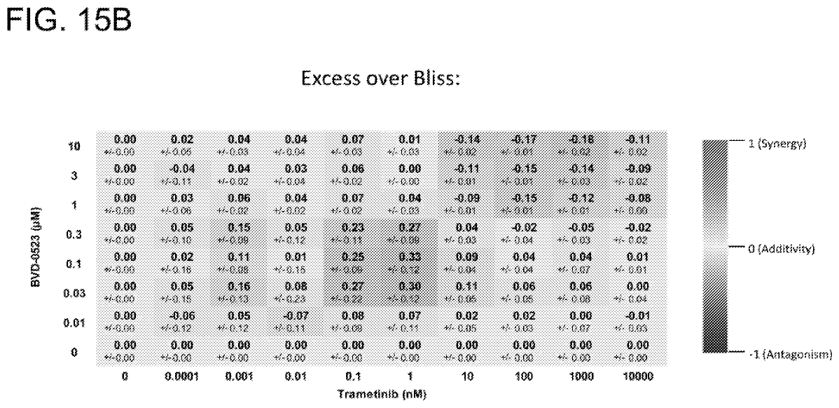

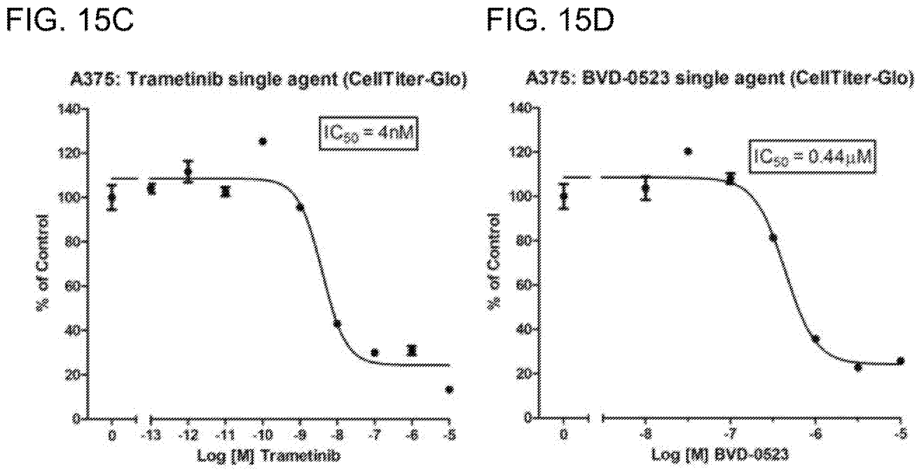

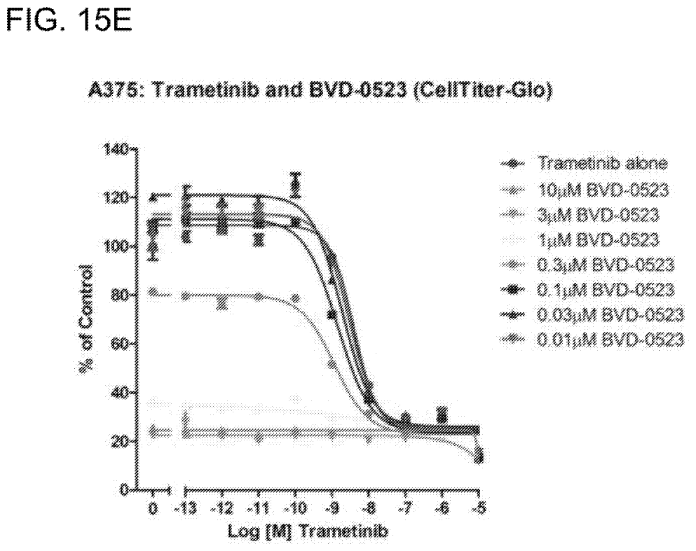

As expected, A375 cells, which carry a BRAF (V600E) mutation, were sensitive to dabrafenib. Single agent IC₅₀ values calculated using Alamar Blue (FIG. 10A-FIG. 10E, FIG. 12A-FIG. 12E, and FIG. 14A-FIG. 14E) were generally slightly lower for Dabrafenib and BVD-523 compared to those derived using CellTiter-Glo (FIG. 11A-FIG. 11E, FIG. 13A-FIG. 13E, and FIG. 15A-FIG. 15E). Published IC₅₀ values for Dabrafenib and Trametinib in a 72 hour CellTiter-Glo assay were 28±16 nM and 5±3 nM respectively (Greger et al., 2012; King et al., 2013)—the single agent results reported here are consistent with these values. There was some evidence for a window of synergy in all treatments. Variation between triplicates was low, however, there was some evidence of edge effects that likely explains the apparent enhanced growth observed in some treatments versus the no drug control (e.g. particularly apparent in the Trametinib/BVD-523 combination). This makes the interpretation of the Bliss analysis more challenging as in some treatments it may have resulted in the artefactual enhancement in the level of synergy.

The combination assays were repeated for A375 cells. Single agent BVD-523, Trametinib and Dabrafenib potencies were consistent with those reported in the previous studies disclosed herein.

In sum, taken together the data show that MEK and BRAF resistant cells could be overcome by treatment with the ERK inhibitor, BVD-523.

### Example 6

### BVD-523 Altered Markers of MAPK Kinase Activity and Effector Function

For Western blot studies, HCT116 cells (5×10⁶) were seeded into 10 cm dishes in McCoy's 5A plus 10% FBS. A375 cells (2.5×10⁶) were seeded into 10 cm dishes in DMEM plus 10% FBS. Cells were allowed to adhere overnight prior to addition of the indicated amount of test compound (BVD-523) or vehicle control. Cells were treated for either 4 or 24 hours before isolation of whole-cell protein lysates, as specified below. Cells were harvested by trypsinisation, pelleted and snap frozen. Lysates were prepared with RIPA (Radio-Immunoprecipitation Assay) buffer, clarified by centrifugation and quantitated by bicinchoninic acid assay (BCA) assay. 20-50 μg of protein was resolved by SDS-PAGE electrophoresis, blotted onto PVDF membrane and probed using the antibodies detailed in Table 13 (for the 4-hour treatment) and Table 14 (for the 24-hour treatment) below.

TABLE 13
Antibody Details

| | | | | | Incubation/ | |
|---|---|---|---|---|---|---|
| | Size | | | | Block | |
| Antigen | (kDa) | Supplier | Cat No | Dilution | Conditions | Secondary |
| pRSK1/2 | 90 | Cell | 9335 | 1:1000 | o/n 4° C. 5% | anti-rabbit |
| pS380 | | Signaling | | | BSA | |
| pRSK1/2 | 90 | Cell | 11989 | 1:2000 | o/n 4° C. 5% | anti-rabbit |
| pS380 | | Signaling | | | BSA | |
| pRSK- | 90 | Millipore | 04-419 | 1:40000 | o/n 4° C. 5% | anti-rabbit |
| T359/S363 | | | | | BSA | |
| Total RSK | 90 | Cell | 9333 | 1:1000 | o/n 4° C. 5% | anti-rabbit |
| | | Signaling | | | BSA | |
| pErk 1/2 | 42/44 | Cell | 9106S | 1:500 | o/n 4° C. 5% | anti-mouse |
| | | Signaling | | | milk | |
| Total ERK | 42/44 | Cell | 9102 | 1:2000 | o/n 4° C. 5% | anti-rabbit |
| | | Signaling | | | milk | |
| pMEK1/2 | 45 | Cell | 9154 | 1:1000 | o/n 4° C. 5% | anti-rabbit |
| | | Signaling | | | BSA | |
| Total MEK | 45 | Cell | 9126 | 1:1000 | o/n 4° C. 5% | anti-rabbit |
| | | Signaling | | | BSA | |
| pS6- | 32 | Cell | 2211S | 1:3000 | o/n 4° C. 5% | anti-rabbit |
| pS235 | | Signaling | | | milk | |
| Total S6 | 32 | Cell | 2217 | 1:2000 | o/n 4° C. 5% | anti-rabbit |
| | | Signaling | | | milk | |
| DUSP6 | 48 | Cell | 3058S | 1:1000 | o/n 4° C. 5% | anti-rabbit |
| | | Signaling | | | BSA | |
| Total | 73 | BD Bio- | 610152 | 1:2000 | o/n 4° C. 5% | anti-mouse |
| CRAF | | sciences | | | milk | |
| pCRAF- | 73 | Cell | 9427 | 1:1000 | o/n 4° C. 5% | anti-rabbit |
| Ser338 | | Signaling | | | BSA | |
| pRB | 105 | Cell | 9307 | 1:2000 | o/n 4° C. 5% | anti-rabbit |
| (Ser780) | | Signaling | | | BSA | |
| β-Actin | 42 | Sigma | A5441 | 1:500,000 | o/n 4° C. 5% | anti-mouse |
| | | | | | milk | |

TABLE 14
Antibody details

| | | | | | Incubation/ | |
|---|---|---|---|---|---|---|
| | Size | | | | Block | |
| Antigen | (kDa) | Supplier | Cat No | Dilution | Conditions | Secondary |
| pRB | 105 | Cell | 9307 | 1:2000 | o/n 4° C. 5% | anti-rabbit |
| (Ser780) | | Signaling | | | BSA | |
| CCND1 | 34 | Abcam | ab6152 | 1:500 | o/n 4° C. 5% | anti-mouse |
| | | | | | milk | |
| Bim-EL | 23 | Millipore | AB17003 | 1:1000 | o/n 4° C. 5% | anti-rabbit |
| | | | | | BSA | |
| Bim-EL | 23 | Cell | 2933 | 1:1000 | o/n 4° C. 5% | anti-rabbit |
| | | Signaling | | | BSA | |
| BCL-xL | 30 | Cell | 2762 | 1:2000 | o/n 4° C. 5% | anti-rabbit |
| | | Signaling | | | BSA | |
| PARP | 116/89 | Cell | 9542 | 1:1000 | o/n 4° C. 5% | anti-rabbit |
| | | Signaling | | | milk | |
| Cleaved | 17, 19 | Cell | 9664X | 1:1000 | o/n 4° C. 5% | anti-rabbit |
| Caspase 3 | | Signaling | | | milk | |
| DUSP6 | 48 | Cell | 3058S | 1:1000 | o/n 4° C. 5% | anti-rabbit |
| | | Signaling | | | BSA | |
| pRSK1/2 | 90 | Cell | 9335 | 1:1000 | o/n 4° C. 5% | anti-rabbit |
| pS380 | | Signaling | | | BSA | |
| pRSK1/2 | 90 | Cell | 11989 | 1:2000 | o/n 4° C. 5% | anti-rabbit |
| pS380 | | Signaling | | | BSA | |
| pRSK- | 90 | Millipore | 04-419 | 1:40000 | o/n 4° C. 5% | anti-rabbit |
| T359/S363 | | | | | BSA | |
| Total RSK | 90 | Cell | 9333 | 1:1000 | o/n 4° C. 5% | anti-rabbit |
| | | Signaling | | | BSA | |
| pErk 1/2 | 42/44 | Cell | 9106S | 1:500 | o/n 4° C. 5% | anti-mouse |
| | | Signaling | | | milk | |
| Total ERK | 42/44 | Cell | 9102 | 1:2000 | o/n 4° C. 5% | anti-rabbit |
| | | Signaling | | | milk | |
| B-Actin | 42 | Sigma | A5441 | 1:500,000 | o/n 4° C. 5% | anti-mouse |
| | | | | | milk | |

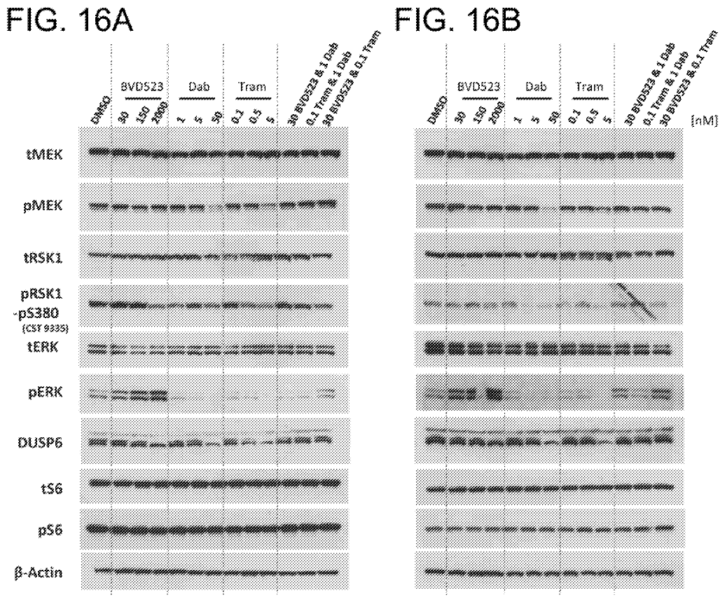

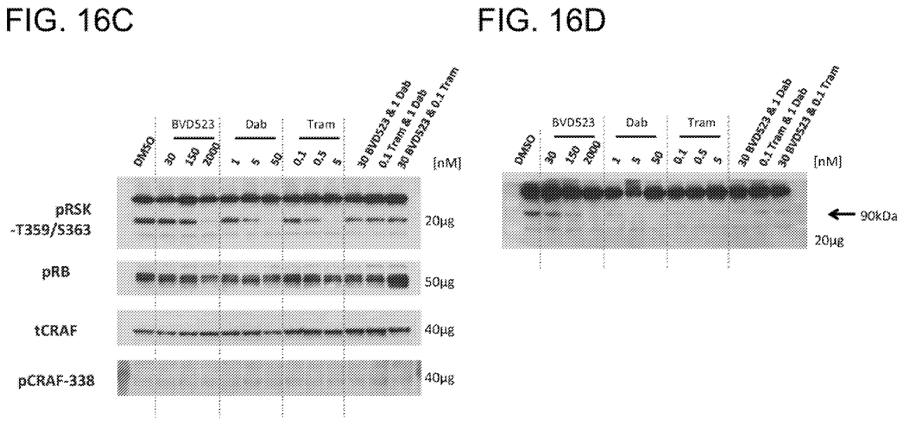

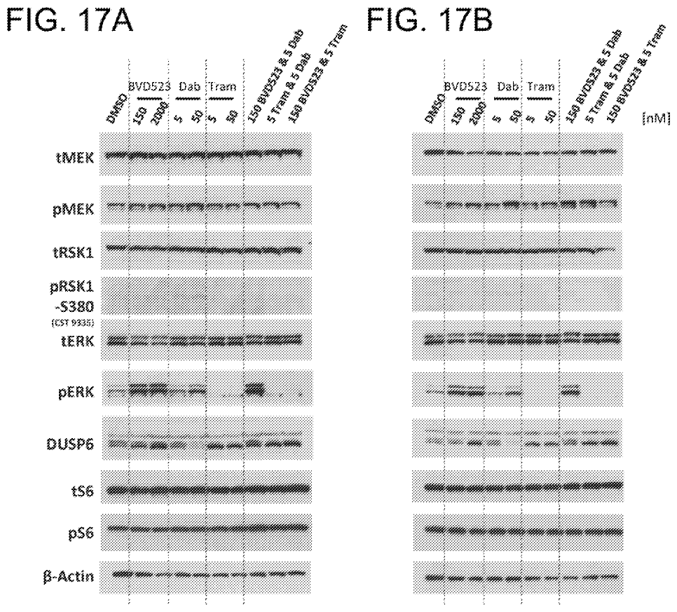

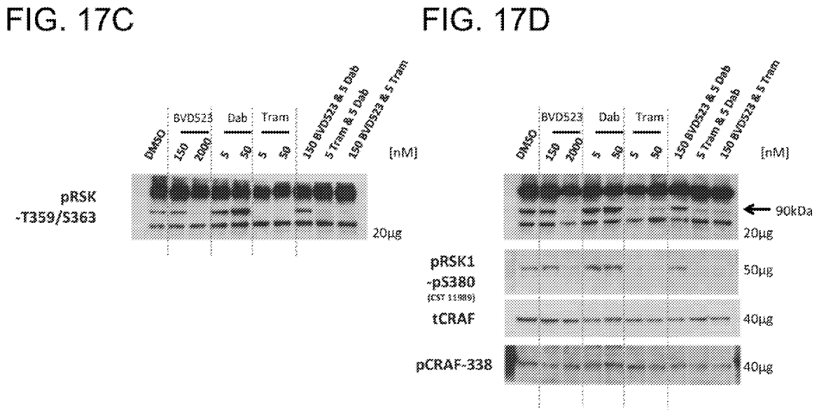

FIG. 16A-FIG. 16D, FIG. 17A-FIG. 17D, and FIG. 18A-FIG. 18D show Western blot analyses of cells treated with BVD-523 at various concentrations for the following: 1) MAPK signaling components in A375 cells after 4 hours; 2) cell cycle and apoptosis signaling in A375 24 hours treatment with various amounts of BVD-523; and 3) MAPK signaling in HCT-116 cells treated for 4 hours. The results show that acute and prolonged treatment with BVD-523 in RAF and RAS mutant cancer cells in-vitro affects both substrate phosphorylation and effector targets of ERK kinases. The concentrations of BVD-523 required to induce these changes is typically in the low micromolar range.

Changes in several specific activity markers are noteworthy. First, the abundance of slowly migrating isoforms of ERK kinase increase following BVD-523 treatment; modest changes can be observed acutely, and increase following prolonged treatment. While this could indicate an increase in enzymatically active, phosphorylated forms of ERK, it remains noteworthy that multiple proteins subject to both direct and indirect regulation by ERK remain “off” following BVD-523 treatment. First, RSK1/2 proteins exhibit reduced phosphorylation at residues that are strictly dependent on ERK for protein modification (T359/S363). Second, BVD-523 treatment induces complex changes in the MAPK feedback phosphatase, DUSP6: slowly migrating protein isoforms are reduced following acute treatment, while total protein levels are greatly reduced following prolonged BVD-523 treatment. Both of these findings are consistent with reduced activity of ERK kinases, which control DUSP6 function through both post-translational and transcriptional mechanisms. Overall, despite increases in cellular forms of ERK that are typically thought to be active, it appears likely that cellular ERK enzyme activity is fully inhibited following either acute or prolonged treatment with BVD-523.

Consistent with these observations, effector genes that require MAPK pathway signaling are altered following treatment with BVD-523. The G1/S cell-cycle apparatus is regulated at both post-translational and transcriptional levels by MAPK signaling, and cyclin-D1 protein levels are greatly reduced following prolonged BVD-523 treatment. Similarly, gene expression and protein abundance of apoptosis effectors often require intact MAPK signaling, and total levels of Bim-EL increase following prolonged BVD-523 treatment. As noted above, however, PARP protein cleavage and increased apoptosis were not noted in the A375 cell background; this suggests that additional factors may influence whether changes in BVD-523/ERK-dependent effector signaling are translated into definitive events such as cell death and cell cycle arrest.

Consistent with the cellular activity of BVD-523, marker analysis suggests that ERK inhibition alters a variety of molecular signaling events in cancer cells, making them susceptible to both decreased cell proliferation and survival.

In sum, FIG. 16A-FIG. 16D, FIG. 17A-FIG. 17D, and FIG. 18A-FIG. 18D show that BVD-523 inhibits the MAPK signaling pathway and may be more favorable compared to RAF or MEK inhibition in this setting.

Finally, properties of BVD-523 may make this a preferred agent for use as an ERK inhibitor, compared to other agents with a similar activity. It is known that kinase inhibitor drugs display unique and specific interactions with their enzyme targets, and that drug efficacy is strongly influenced by both the mode of direct inhibition, as well as susceptibility to adaptive changes that occur following treatment. For example, inhibitors of ABL, KIT, EGFR and ALK kinases are effective only when their cognate target is found in active or inactive configurations. Likewise, certain of these inhibitors are uniquely sensitive to either secondary genetic mutation, or post-translational adaptive changes, of the protein target. Finally, RAF inhibitors show differential potency to RAF kinases present in certain protein complexes and/or subcellular localizations. In summary, as ERK kinases are similarly known to exist in diverse, variable, and complex biochemical states, it appears likely that BVD-523 may interact with and inhibit these targets in a fashion that is distinct and highly preferable to other agents.

### Example 7

### Effects of BVD-523 and Benchmark ERK BRAF and MEK Inhibitors on Viability and MAPK Signalling

### Single Agent Proliferation Assay

Cells were seeded in 96-well plates at the densities indicated in Table 15 in McCoy's 5A containing 10% FBS and allowed to adhere overnight prior to addition of compound or vehicle control. Compounds were prepared from DMSO stocks to give the desired final concentrations. The final DMSO concentration was constant at 0.1%. Test compounds were incubated with the cells for 96 h at 37° C., 5% CO₂ in a humidified atmosphere. CellTiter-Glo® reagent (Promega, Madison, WI) was added according to manufacturer's instructions and luminescence detected using the BMG FLUOstar plate reader (BMG Labtech, Ortenberg, Germany). The average media only background value was deducted and the data analysed using a 4-parameter logistic equation in GraphPad Prism (GraphPad Software, La Jolla, CA).

### Combination Proliferation Assay

Cells were seeded into triplicate 96-well plates at the densities indicated in Table 15 in McCoy's 5A containing 10% FBS and allowed to adhere overnight prior to addition of test compound or vehicle control. Combinations were tested using a 10×8 dose matrix. The final DMSO concentration was constant at 0.2%.

Test compounds were incubated with the cells for 96 h at 37° C., 5% CO₂ in a humidified atmosphere. Cells were stained with Hoechst stain and fluorescence detected as described above. The average media only background value was deducted and the data analysed.

Combination interactions across the dose matrix were determined by the Loewe Additivity and Bliss independence models using Chalice™ Combination Analysis Software (Horizon Discovery Group, Cambridge, MA) as outlined in the user manual (available at chalice.horizondiscovery.com/chalice-portal/documentation/analyzer/home.jsp). Synergy is determined by comparing the experimentally observed level of inhibition at each combination point with the value expected for additivity, which is derived from the single-agent responses along the edges of the matrix. Potential synergistic interactions were identified by displaying the calculated excess inhibition over that predicted as being additive across the dose matrix as a heat map, and by reporting a quantitative ‘Synergy Score’ based on the Loewe model. The single agent data derived from the combination assay plates were presented as dose-response curves generated in Chalice™

TABLE 15
Cell Line Seeding Density

| | Seeding density (cell/well) | | |
|---|---|---|---|
| | 96-well | | 10 cm |
| | Prolifer- | 6-Well | dish |
| Cell Line | ation | Western | Westerns |
| RKO Parental | 1000 | 1 × 10⁶ | 2.9 × 10⁶ |
| RKO MEK1 | 1250 | Not tested | Not tested |
| (Q56P/+) Clone 1 | | | |
| RKO MEK1 | 1000 | 7.5 × 10⁵ | 2 ×10⁶ |
| (Q56P/+) Clone 2 | | | |

### Western Blotting

Cells were seeded into 6-well plates (Experiment 1) or 10 cm dishes (Experiment 2) at the densities indicated in Table 15 in McCoy's 5A containing 10% FBS and allowed to adhere overnight prior to addition of compound or vehicle control. Test compounds were added and incubated with the cells for 4 or 24 h at 37° C., 5% CO₂ in a humidified atmosphere. Cells were harvested by trypsinisation, pelleted by centrifugation and snap frozen on dry ice.

Lysates were prepared using RIPA buffer (50 mM Tris-hydrochloride, pH 8.0; 150 mM sodium chloride; 1.0% Igepal CA-630 (NP-40); 0.5% sodium deoxycholate; 0.1% sodium dodecyl sulphate; 1×complete EDTA-free protease inhibitor cocktail (Roche, Nutley, NJ; cat 05 892 791 001); 1×phosSTOP phosphatase inhibitor cocktail (Roche Nutley, NJ; cat. 04 906 837 001)) and clarified by centrifugation at 11,000 rpm for 10 min in a bench-top centrifuge.

Total protein in the lysates was quantitated by BCA assay according to the manufacturer's instructions (Pierce™ BCA Protein Assay Kit; Thermo Scientific, Waltham, MA; cat. 23225), boiled in sample buffer (NuPAGE LDS Sample Buffer; (Invitrogen, Carlsbad, CA; cat. NP0007)) and stored at −80° C.

Equal amounts of protein (40 μg) were resolved on NuPAGE 4-12% Bis-Tris gels (Invitrogen, Carlsbad, CA; cat. WG1402BOX) and blotted onto PVDF membranes using iBlot gel transfer stacks (Invitrogen, Carlsbad, CA; cat. IB4010-01) on an iBlot gel transfer device (Invitrogen Carlsbad, CA) according to the manufacturer's instructions.

Blots were probed using the antibodies and block conditions detailed in Table 16. Western blots were developed using Pierce™ ECL2 Western blotting substrate (Thermo Scientific, Waltham, MA; cat. 80196) and imaged using a FluorChem M Western blot imager (ProteinSimple, San Jose, CA).

TABLE 16
Antibodies and Western Blotting Conditions

| | | | | | Incubation/ | |
|---|---|---|---|---|---|---|
| | Size | | | | block | |
| Antigen | (kDa) | Supplier | Cat No | Dilution | Conditions | Secondary |
| pRSK- | 90 | Millipore | 04-419 | 1:20000 | o/n 4° C. 5% | anti-rabbit |
| T359/S363 | | | | | BSA | |
| Total RSK | 90 | Cell | 9333 | 1:1000 | o/n 4° C. 5% | anti-rabbit |
| | | Signaling | | | BSA | |
| pErk 1/2 | 42/44 | Cell | 9106S | 1:500 | o/n 4° C. 5% | anti-mouse |
| | | Signaling | | | milk | |
| Total ERK | 42/44 | Cell | 9102 | 1:2000 | o/n 4° C. 5% | anti-rabbit |
| | | Signaling | | | milk | |
| pMEK1/2 | 45 | Cell | 9154 | 1:1000 | o/n 4° C. 5% | anti-rabbit |
| | | Signaling | | | BSA | |
| Total MEK | 45 | Cell | 9126 | 1:1000 | o/n 4° C. 5% | anti-rabbit |
| | | Signaling | | | BSA | |
| DUSP6 | 48 | Cell | 3058S | 1:1000 | o/n 4° C. 5% | anti-rabbit |
| | | Signaling | | | BSA | |
| pRB | 105 | Cell | 9307 | 1:2000 | o/n 4° C. 5% | anti-rabbit |
| (Ser780) | | Signaling | | | BSA | |
| CCND1 | 34 | Abcam | ab6152 | 1:500 | o/n 4° C. 5% | anti-mouse |
| | | | | | milk | |
| B-Actin | 42 | Sigma | A5441 | 1:100,000 | o/n 4° C. 5% | anti-mouse |
| | | | | | milk | |
| Anti-rabbit | — | Cell | 7074S | 1:2000 | 1 h room temp; | — |
| HRP-conjugated | | Signaling | | | Block matched to | |
| secondary | | | | | primary Antibody | |
| Anti-mouse | — | Cell | 7076 | 1:5000 | 1 h room temp; | — |
| HRP-conjugated | | Signaling | | | Block matched to | |
| secondary | | | | | primary Antibody | |

The MEK1 (Q56P) mutation exemplifies a class of clinically relevant MEK1/2 activating mutations known to up-regulate the MAPK pathway and drive acquired resistance to BRAF or MEK inhibitors.

This study used a pair of RKO BRAF (V600E) cell lines that are isogenic for the presence or absence of a MEK1 (Q56P) activating mutation, to assess the effect that activating MEK mutations have in response to the novel ERK inhibitor BVD-523 versus other benchmark MAPK inhibitors.

Effects of on cell viability were assessed by quantitating cellular ATP levels using CellTiter-Glo® after 96 h. Single agent assays demonstrated that the double mutant BRAF (V600E)::MEK1 (Q56P) cells displayed a markedly reduced sensitivity to inhibition with benchmark clinical BRAF (exemplified by Dabrafenib) or MEK (exemplified by Trametinib) inhibitors relative to the parental BRAF (V600E) cells, which demonstrates the suitability of this isogenic model for recapitulating the acquired resistance known to be associated with this class of mutation in the clinic (Table 17).

TABLE 17
Single Agent IC₅₀ Values

| | | RKO MEK1 | RKO MEK1 |
|---|---|---|---|
| | RKO | Q56P/+ | Q56P/+ |
| Compound | Parental | CI.1 | CI.2 |
| BVD-523 | 0.20 | 0.17 | 0.18 |
| SCH772984 | 0.04 | 0.14 | 0.12 |
| Dabrafenib | n.d. | n.d. | n.d. |
| Trametinib | 0.006 | 0.093 | 0.080 |
| Paclitaxel | 0.002 | 0.002 | 0.002 |

n.d.—not determined, only a partial dose response achieved

In contrast, response to BVD-523 was identical in both the parental and double mutant cells, indicating that BVD-523 is not susceptible to this mechanism of acquired resistance.

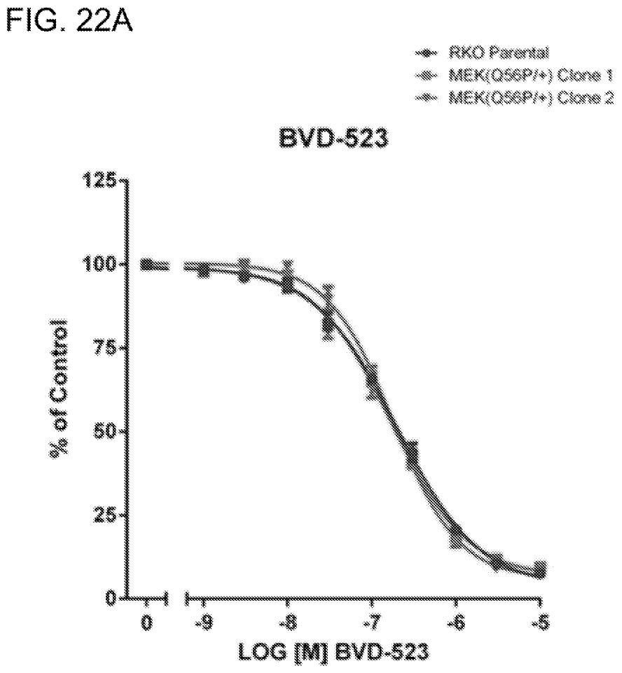

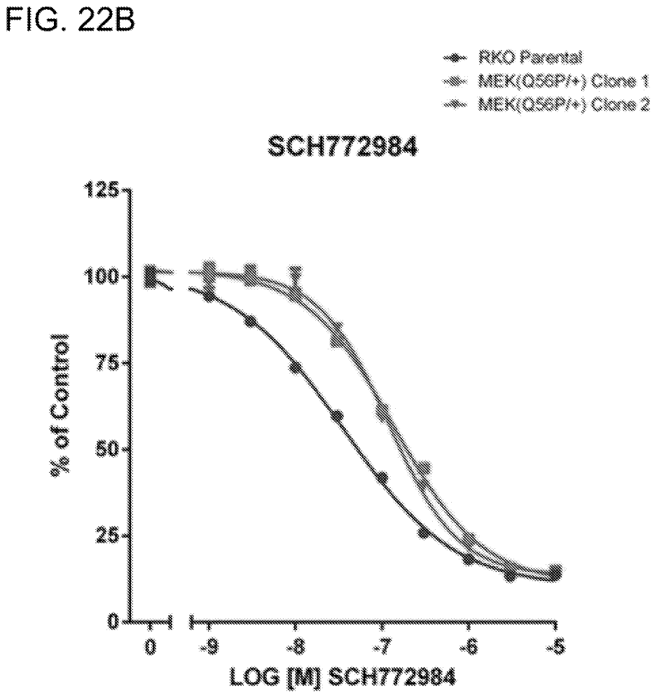

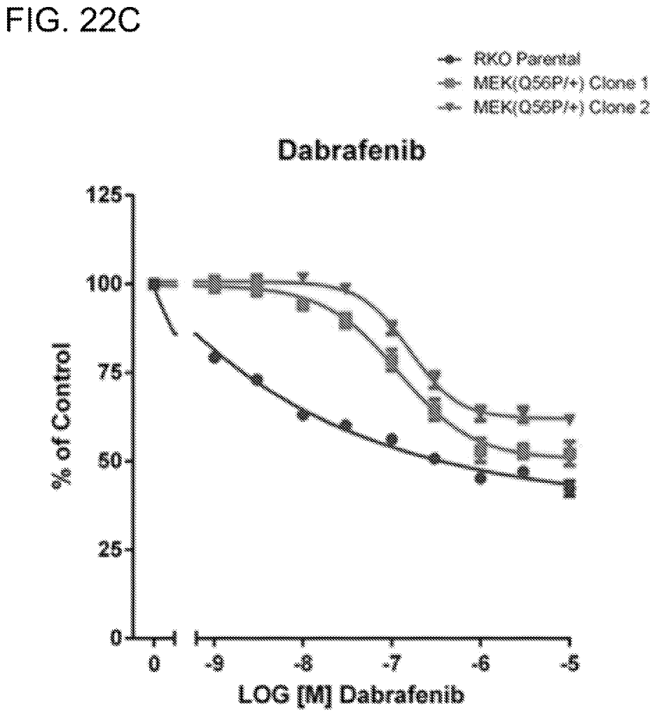

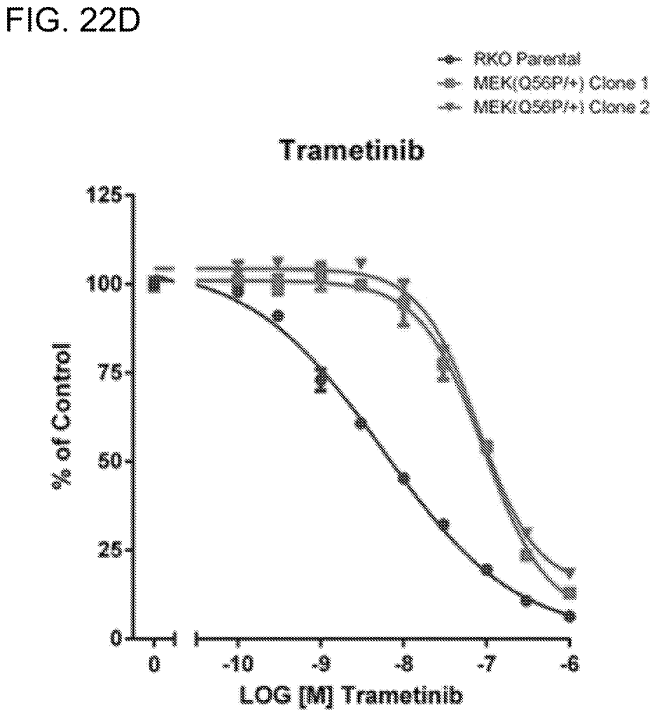

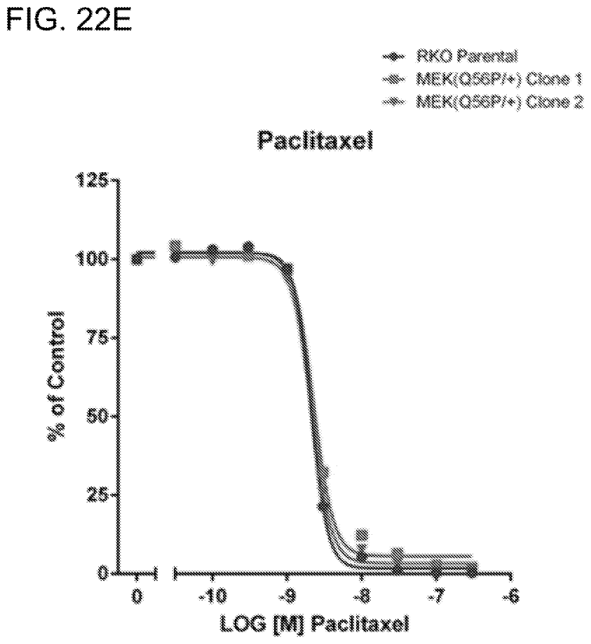

These results were identical in two independently derived double mutant BRAF (V600E)::MEK1 (Q56P) cell line clones confirming that these differences in response versus the parental cells were specifically related to the presence of the MEK1 mutation rather than an unrelated clonal artifact (FIG. 22A-FIG. 22E). Similar results were also observed with a second mechanistically distinct benchmark ERK inhibitor (SCH772984), which supports the notion that these observations are specifically related to inhibition of ERK and not due to an off-target effect.

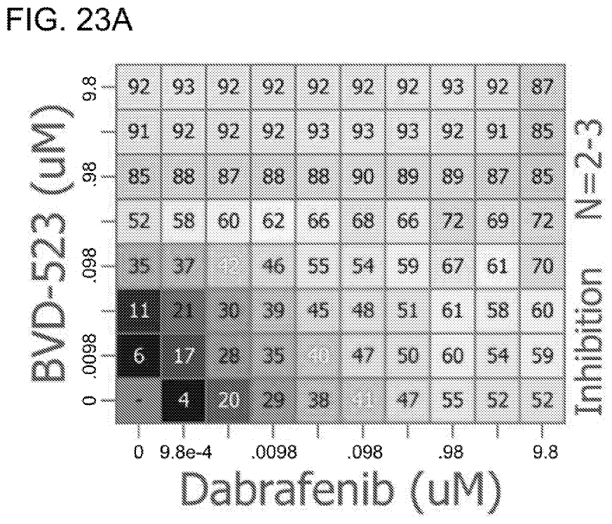

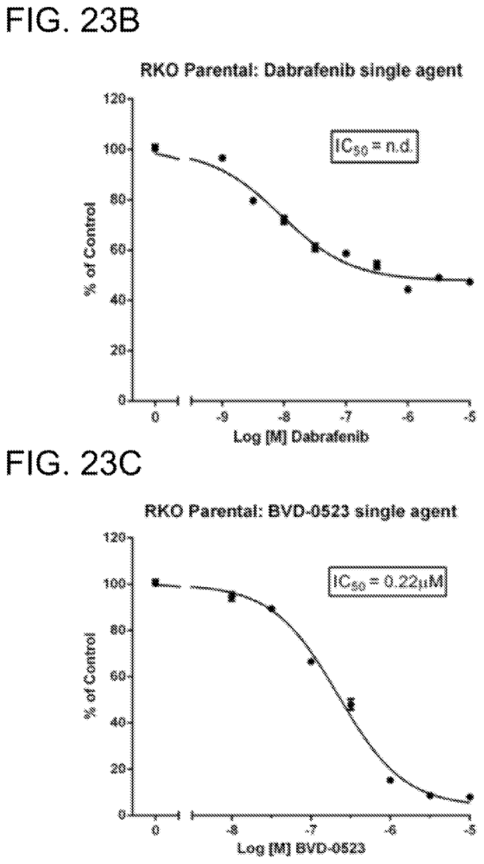

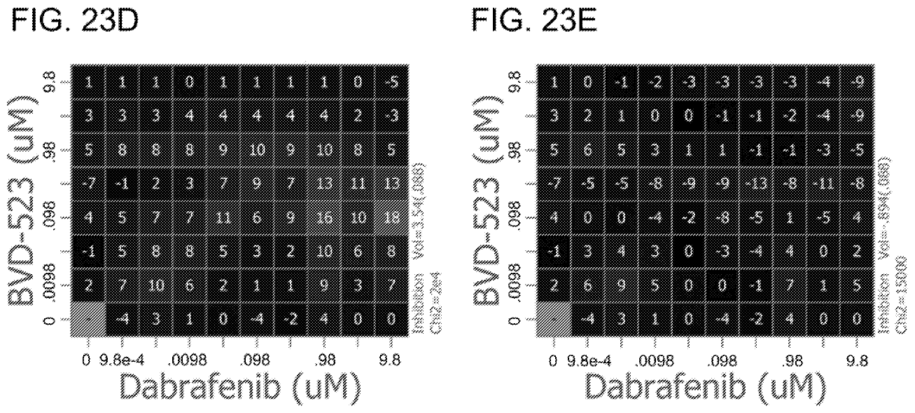

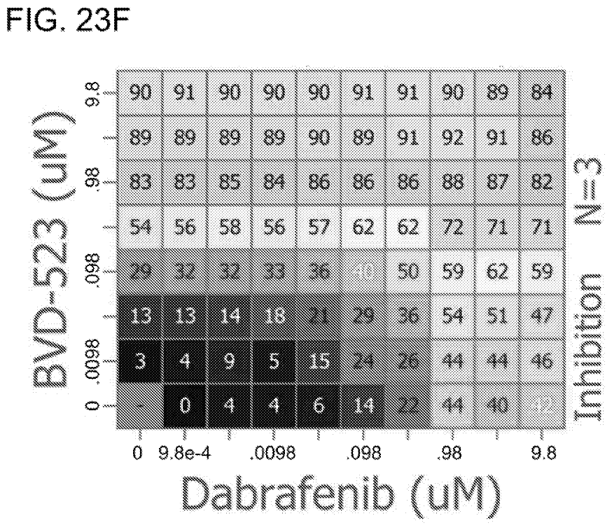

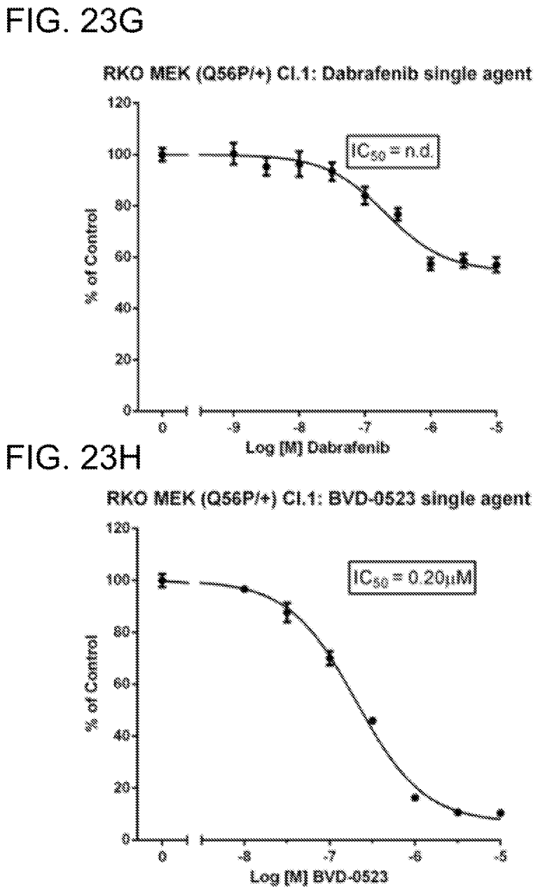

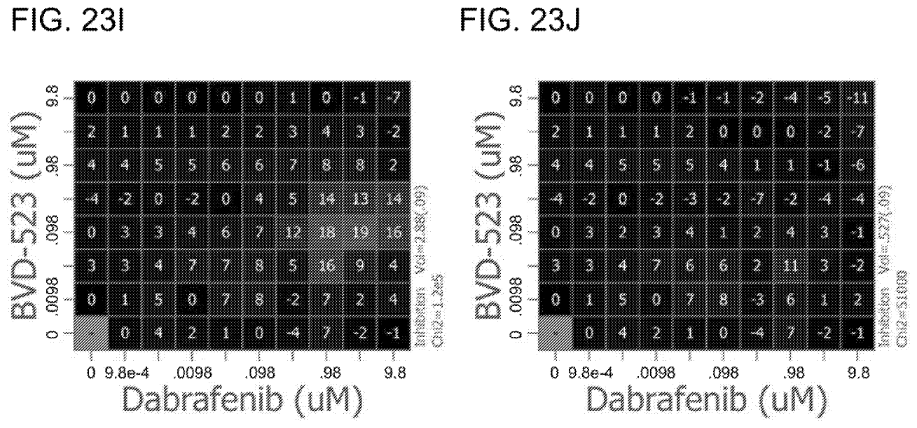

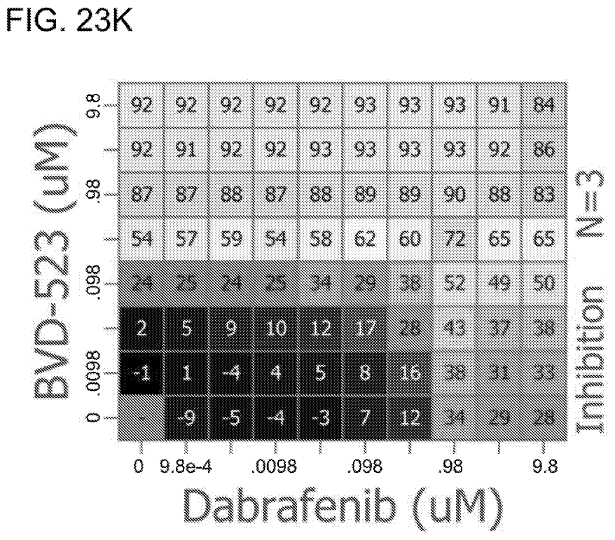

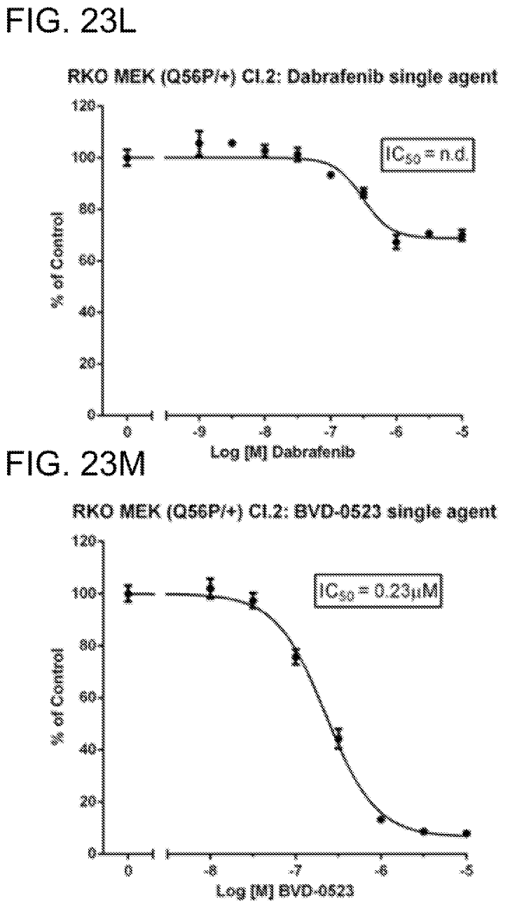

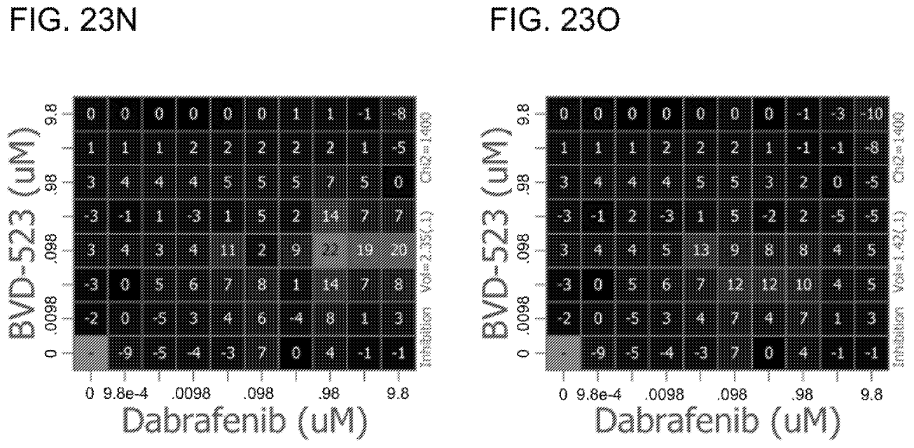

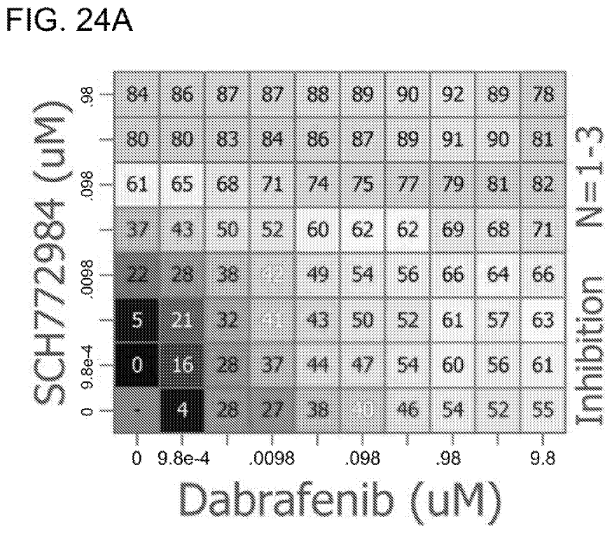

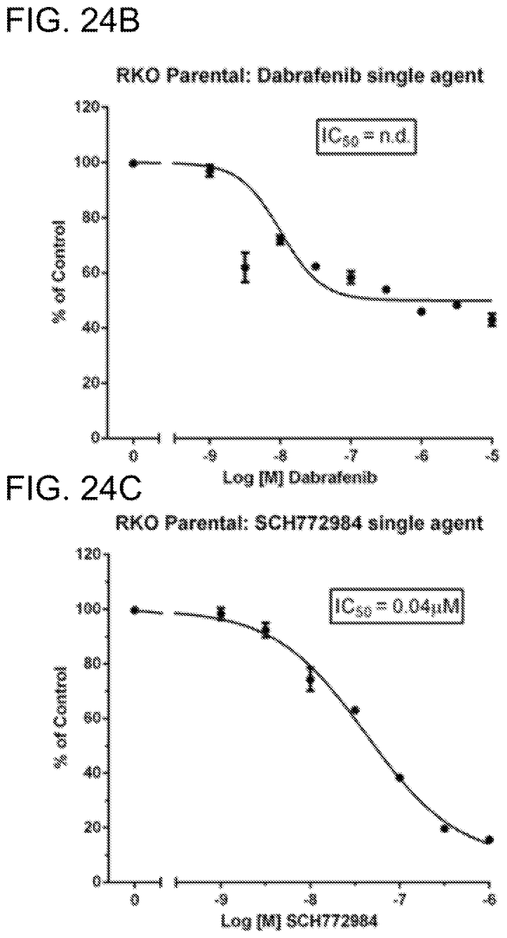

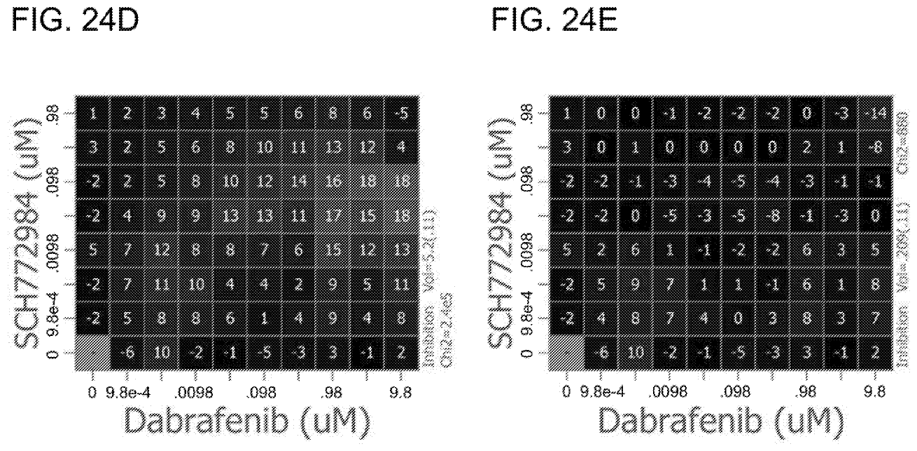

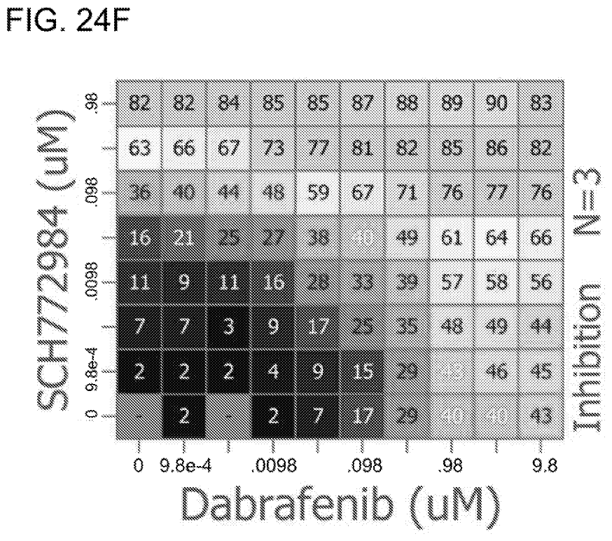

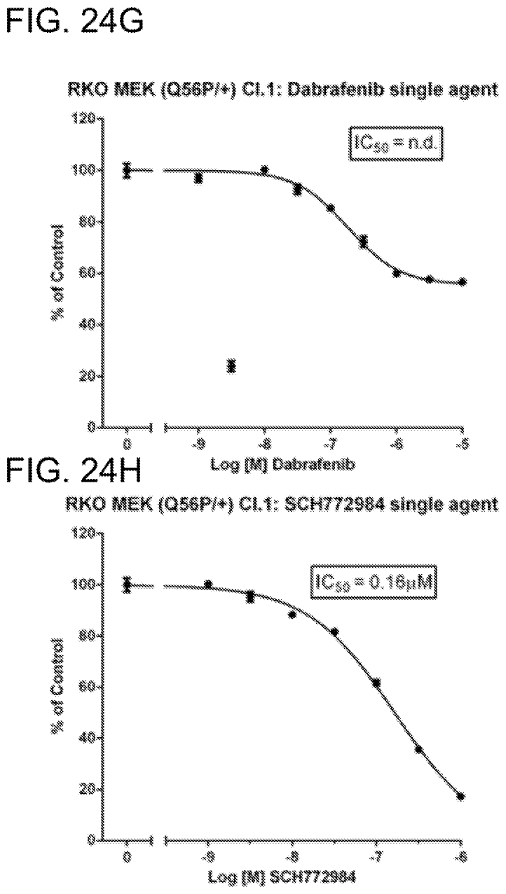

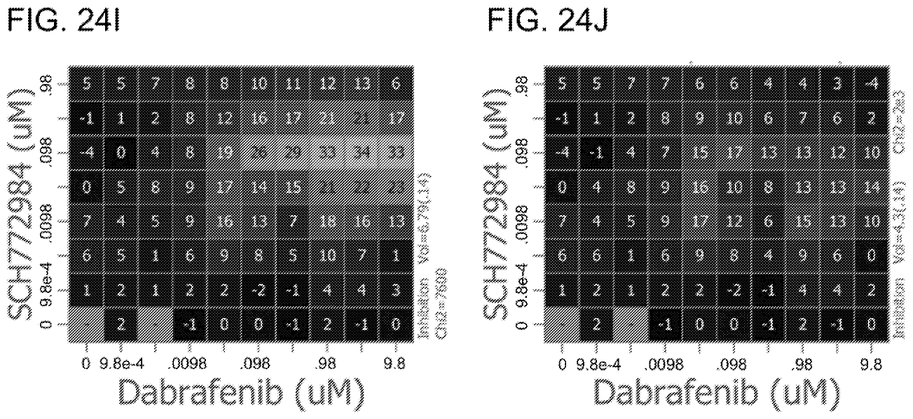

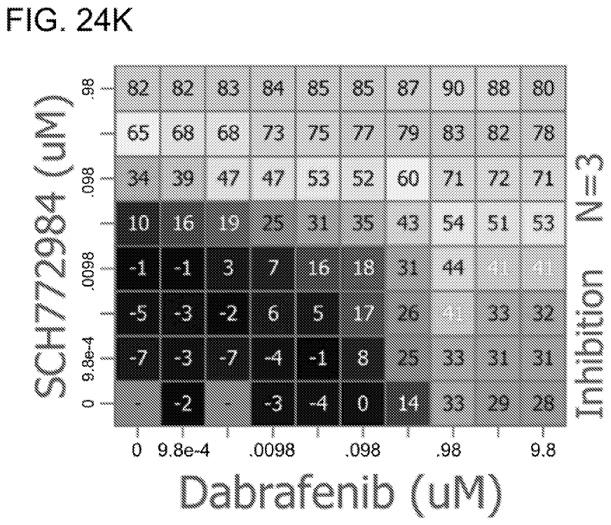

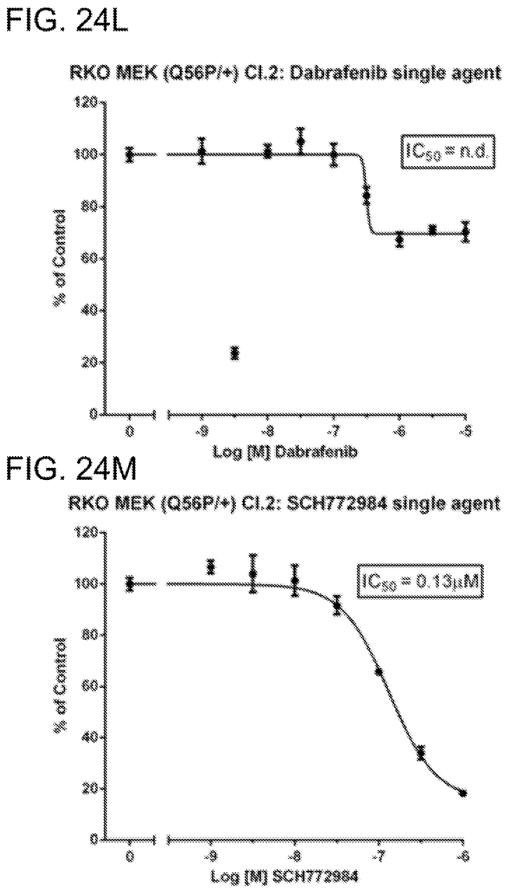

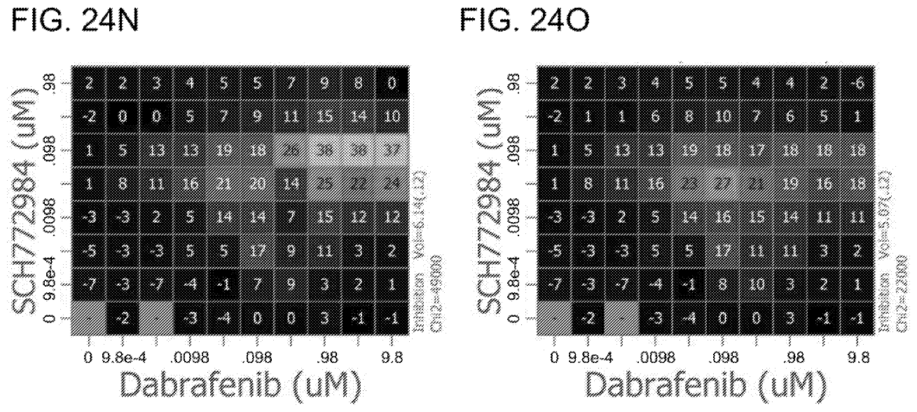

The effect of combining BVD-523 with a BRAF inhibitor (exemplified by Dabrafenib) was also assessed in these cell lines across a matrix of concentrations using the Loewe Addivity or Bliss Independence models with Horizon's Chalice™ combination analysis software (FIG. 23-FIG. 23O and FIG. 24A-FIG. 24O). The presence of potentially synergistic interactions was then assessed by displaying the calculated excess inhibition over that predicted as being additive across the dose matrix as a heat map, and by calculating a ‘Volume Score’ that shows whether the overall response to a combination is synergistic (positive values), antagonistic (negative values) or additive (~0).

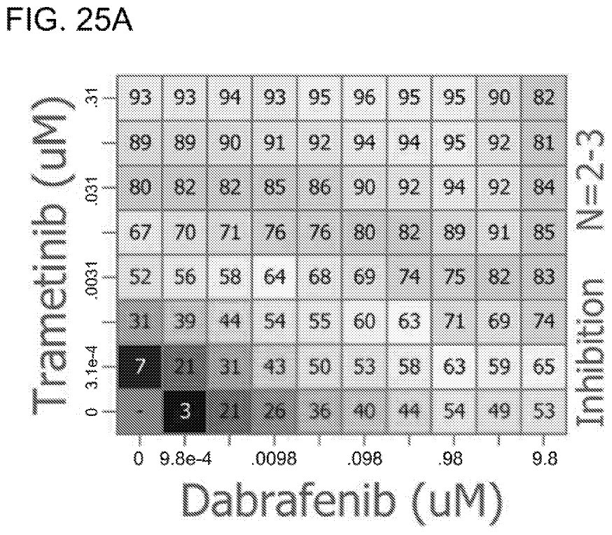

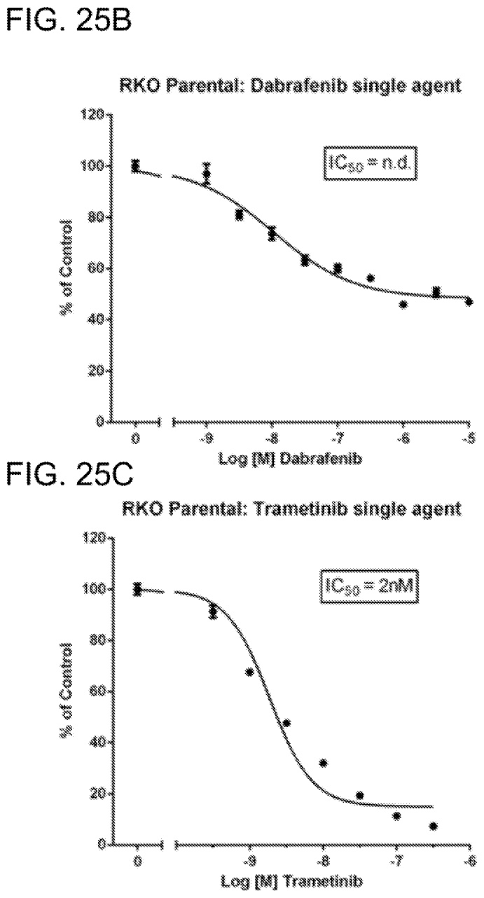

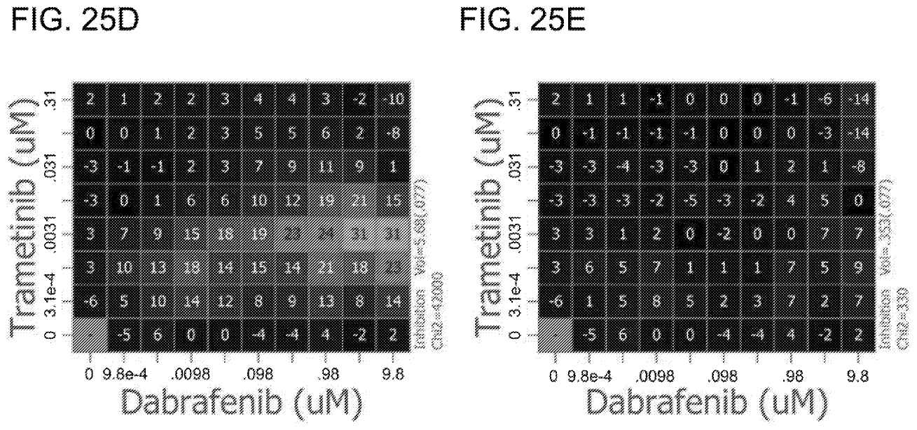

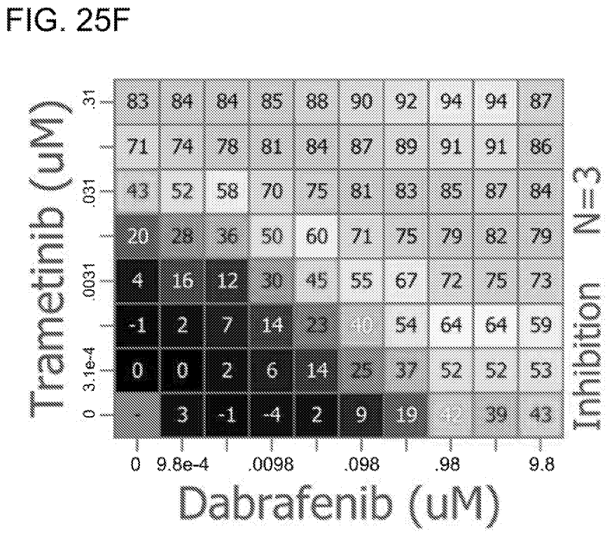

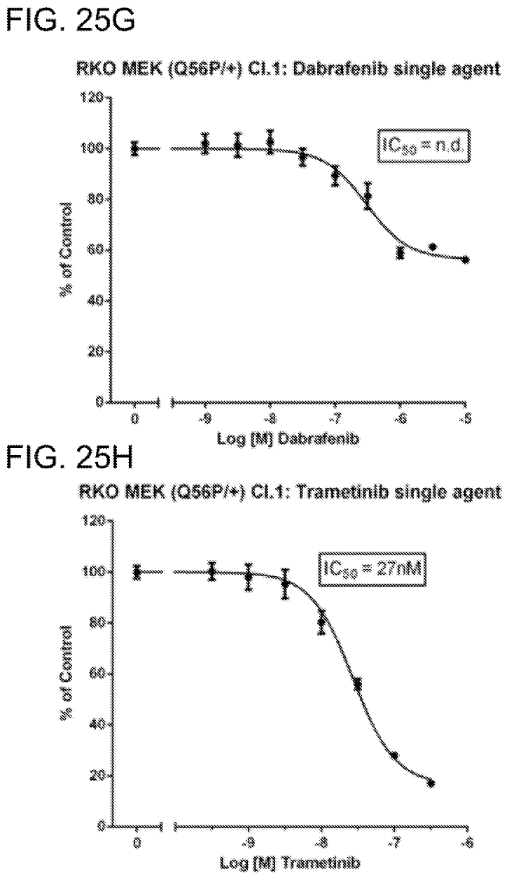

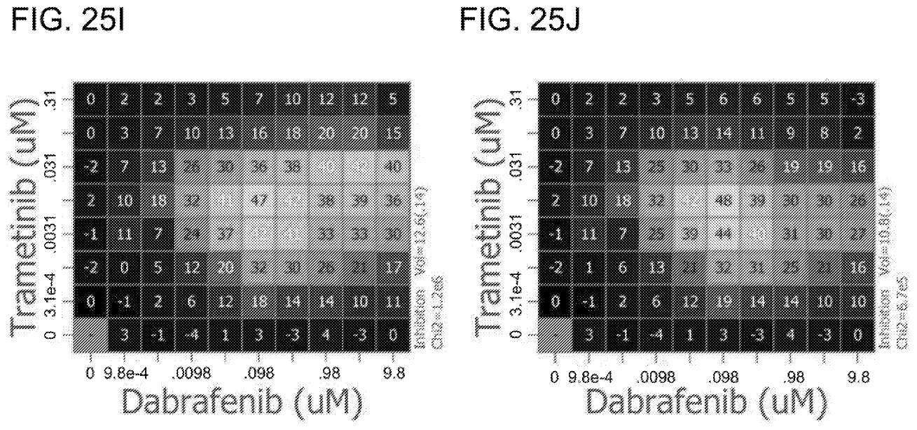

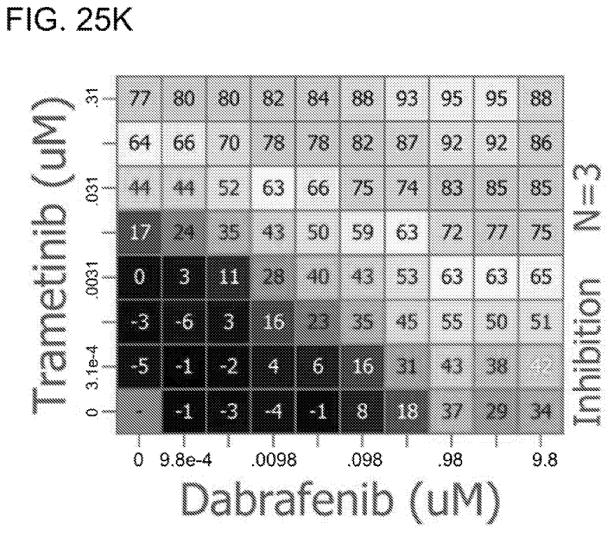

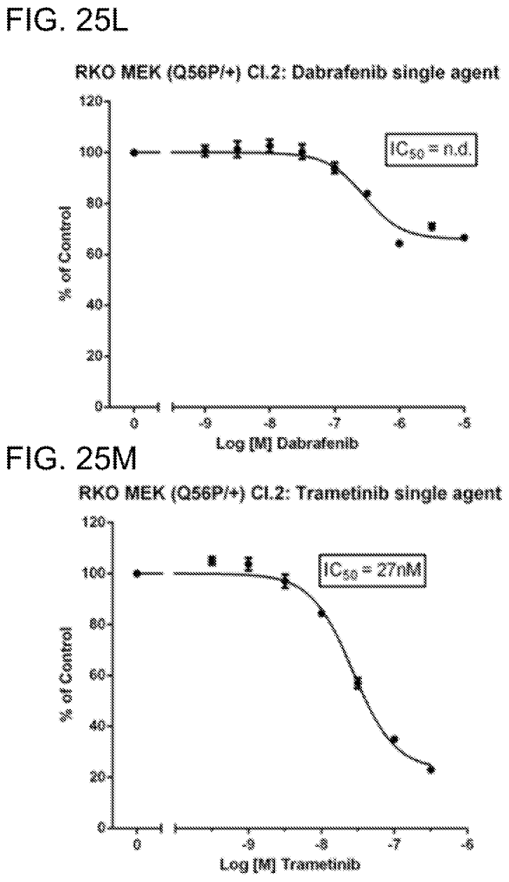

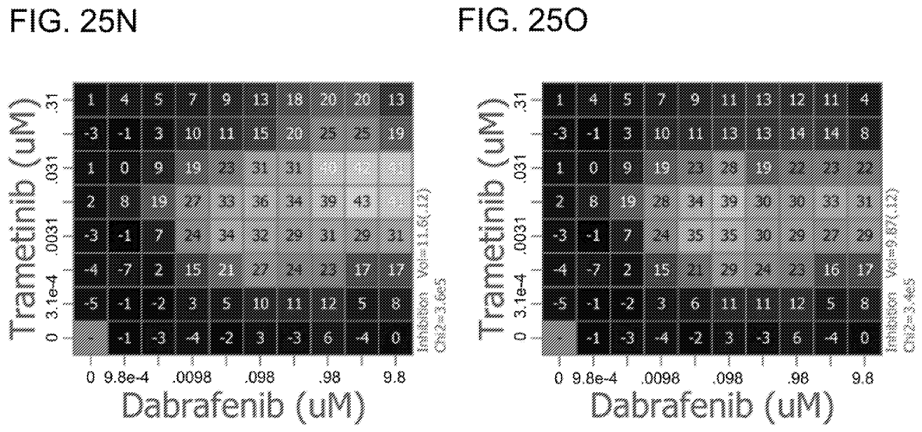

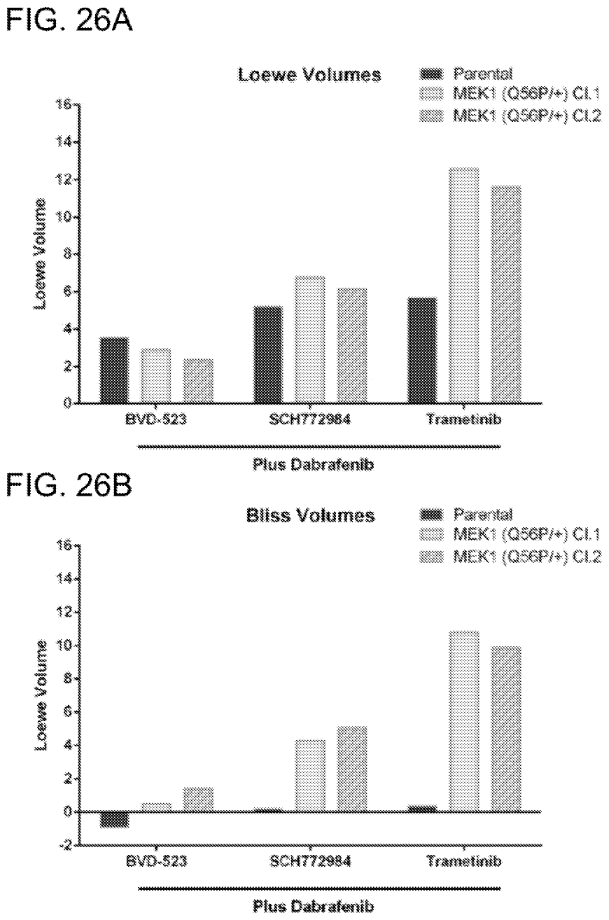

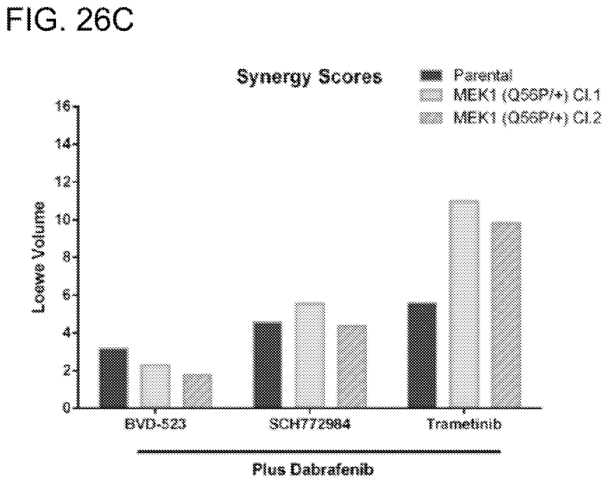

The results suggest that the BVD-523::Dabrafenib combination was mainly additive in the parental and mutant cell line. In contrast, the combination of a MEK inhibitor (trametinib) plus Dabrafenib, while being mostly additive in the parental cell line, showed strong synergy in the double mutant BRAF (V600E)::MEK1 (Q56P) cell line (FIG. 25A-FIG. 25O). Loewe Volumes, Bliss Volumes and Synergy scores for the combinations tested are shown in Tables 18-20, respectively and are shown graphed in FIG. 26A-FIG. 26C.

TABLE 18
Loewe Volumes

| | | RKO MEK1 | RKO MEK1 |
|---|---|---|---|
| | RKO | (Q56P) - | (Q56P) - |
| | Parental | Clone 1 | Clone 2 |
| BVD-523 × Dabrafenib | 3.54 | 2.88 | 2.35 |
| Dabrafenib × SCH772984 | 5.2 | 6.79 | 6.14 |
| Dabrafenib Trannetinib | 5.68 | 12.6 | 11.6 |

TABLE 19
Bliss Volumes

| | | RKO MEK1 | RKO MEK1 |
|---|---|---|---|
| | RKO | (Q56P) - | (Q56P) - |
| | Parental | Clone 1 | Clone 2 |
| BVD-523 × Dabrafenib | −0.894 | 0.527 | 1.42 |
| Dabrafenib × SCH772984 | 0.209 | 4.3 | 5.07 |
| Dabrafenib × Trannetinib | 0.353 | 10.8 | 9.87 |

TABLE 20
Synergy Scores

| | | RKO MEK1 | RKO MEK1 |
|---|---|---|---|
| | RKO | (Q56P) - | (Q56P) - |
| | Parental | Clone 1 | Clone 2 |
| BVD-523 × Dabrafenib | 3.18 | 2.31 | 1.77 |
| Dabrafenib × SCH772984 | 4.56 | 5.57 | 4.36 |
| Dabrafenib × Trannetinib | 5.58 | 11 | 9.83 |

Effects on MAPK pathway signally was assessed by Western blotting. The levels of basal ERK phosphorylation (DMSO samples) was markedly upregulated in the MEK1 (Q56P)-expressing line relative to parental further confirming that this isogenic model faithfully recapitulates the expected phenotype for the expression of MEK activating acquired resistance mutations.

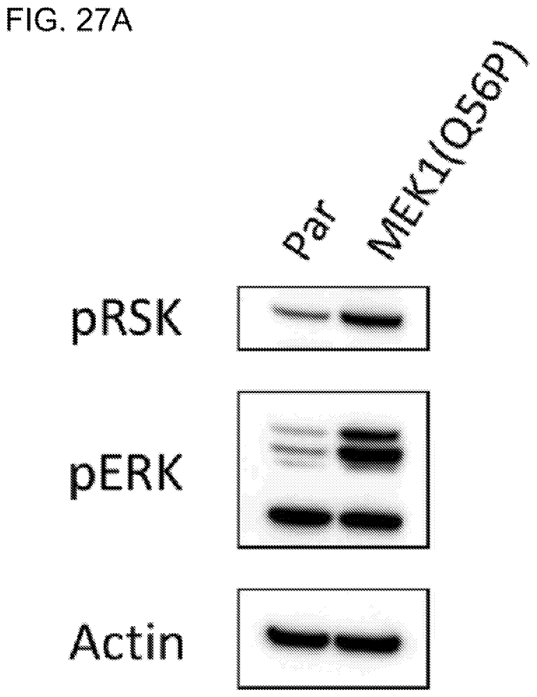

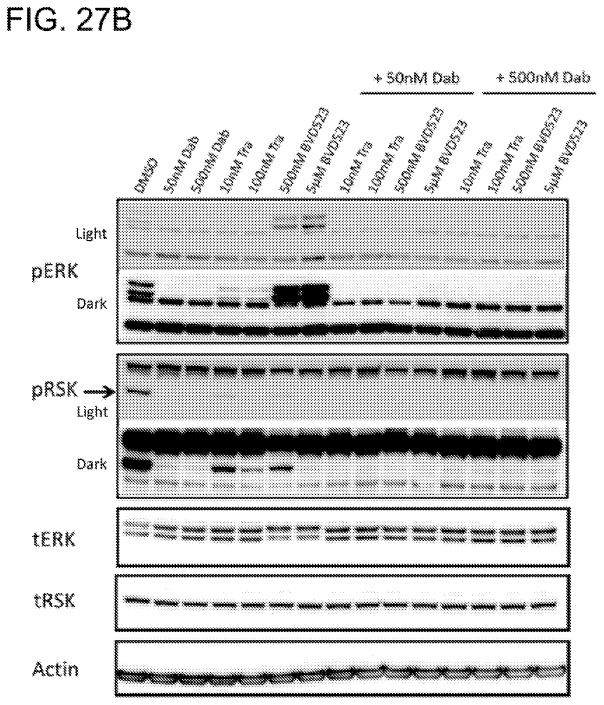

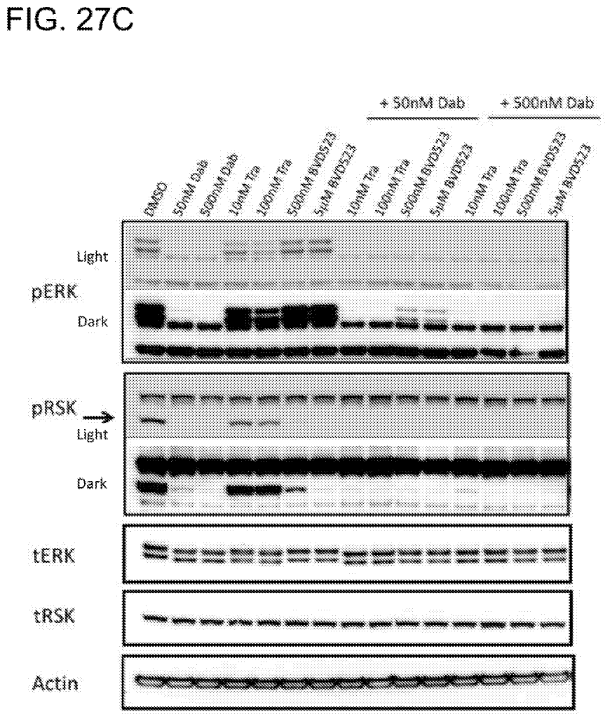

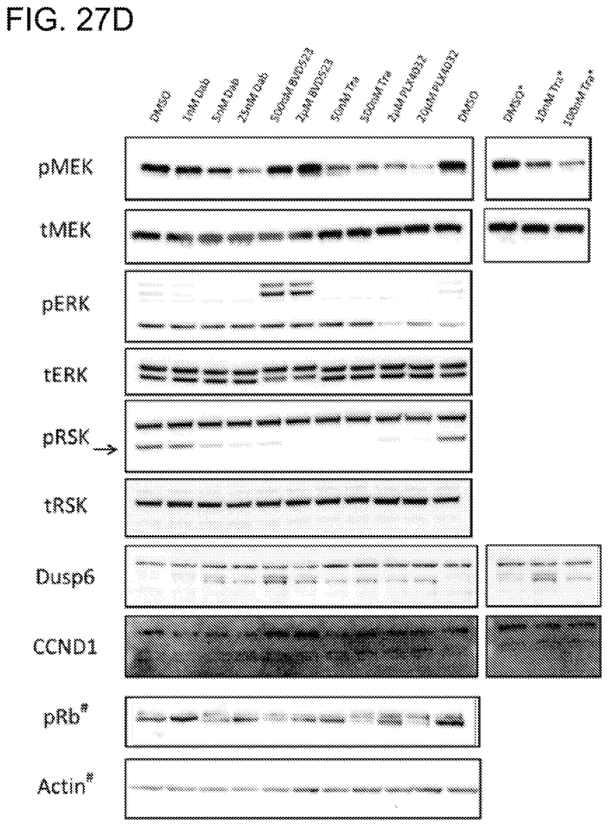

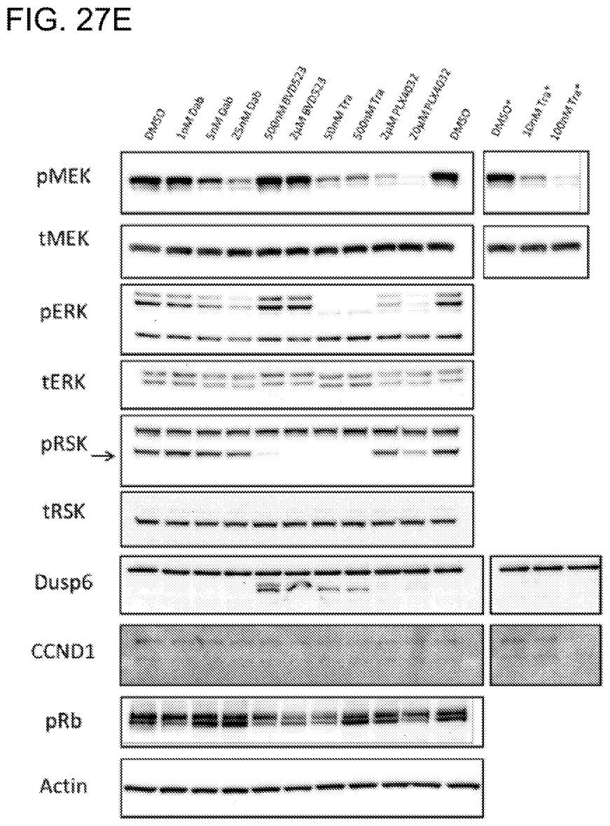

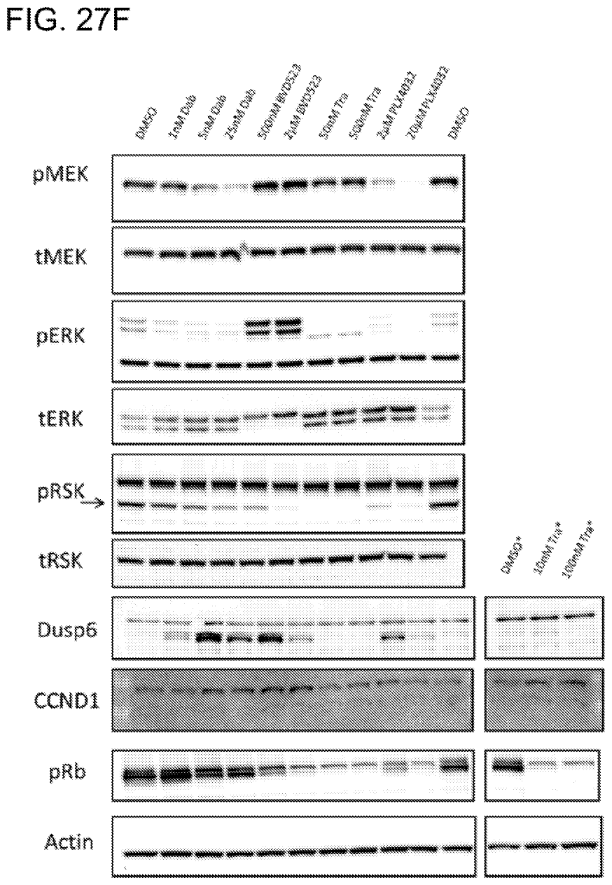

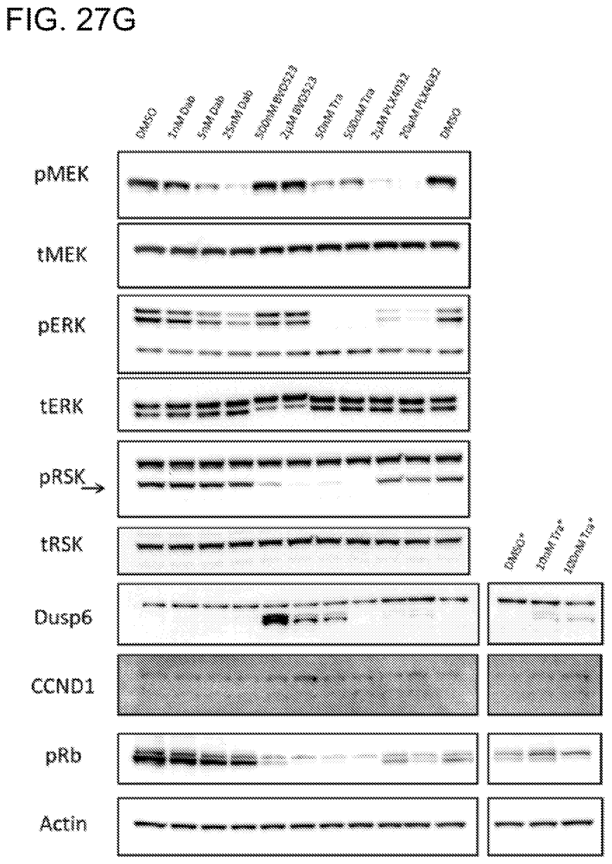

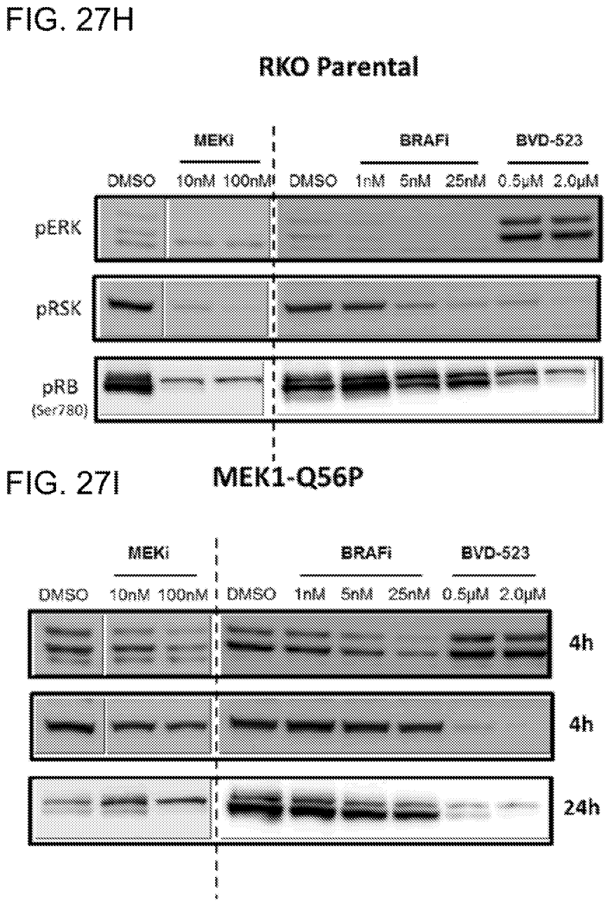

In the parental BRAF (V600E) RKO cells, a reduced level of RSK1/2 phosphorylation is observed following acute treatment with RAF, MEK and ERK kinase inhibitors at pharmacologically active concentrations. In contrast, isogenic, double mutant BRAFV600E::MEK1Q56P cells do not exhibit reduced RSK phosphorylation following BRAF or MEK inhibitor treatment, while BVD-523 remains effective at similar concentrations (FIG. 27A-FIG. 27I). The dotted lines indicate that the trametinib-treated samples (plus matched DMSO control) and blots are derived from a separate experiment to the BRAFi and BVD-523 treated samples.

Changes in effector gene signaling consistent with cell growth inhibition patterns are observed following prolonged inhibitor treatment. In parental RKO lines, a reduced level of phosphorylated pRB is observed following prolonged MEK and ERK inhibitor treatment. At the level of pRB modulation, MEK1 mutant lines appear insensitive to low concentration MEK inhibitor treatment, while higher concentrations remain effective. Critically, BVD-523 potency against pRB activity does not appear to be strongly affected by MEK mutation. Surprisingly, RAF inhibitor treatment does not affect pRB status, despite potent inhibition of upstream signaling, in both parental and MEK mutant backgrounds.

In summary, these results show that BVD-523 is not susceptible to acquired resistance driven by MEK activating mutations such as MEK1 (Q56P). In addition they suggest that in combination the interactions between BVD-523 and BRAFi (exemplified by Dabrafenib) are additive irrespective of the presence of a MEK activating mutation.

### Example 8

### Combination Interactions Between ERK Inhibitors

RAF mutant melanoma cell line A375 cells were cultured in DMEM with 10% FBS and seeded into triplicate 96-well plates at an initial density of 2000 cells per well. Combination interactions between ERK inhibitors BVD-523 and SCH772984 were analized after 72 hours as described above in Example 4. Viability was determined using CellTiter-Glo® reagent (Promega, Madison, WI) according to manufacturer's instructions and luminescence was detected using the BMG FLUOstar plate reader (BMG Labtech, Ortenberg, Germany).

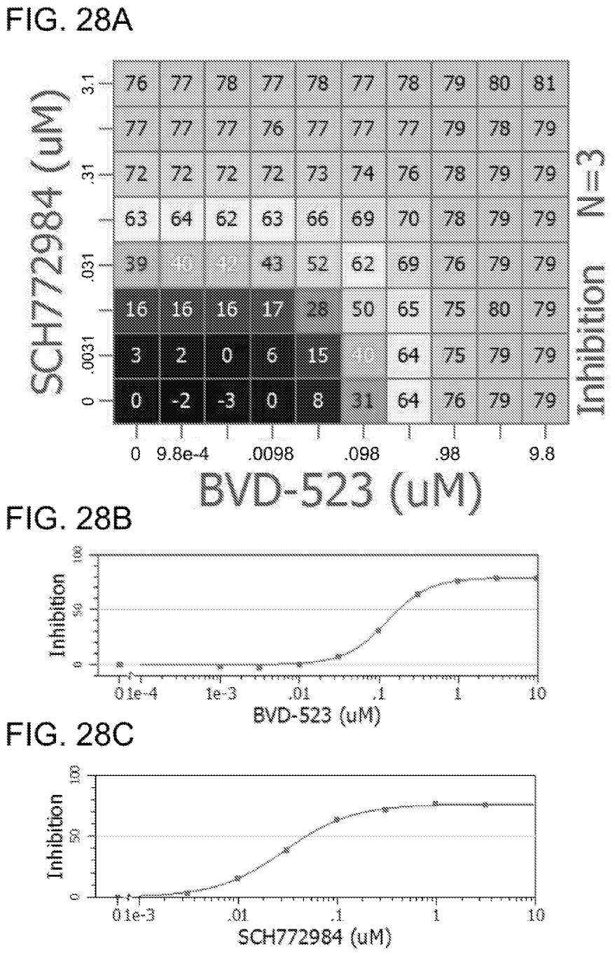

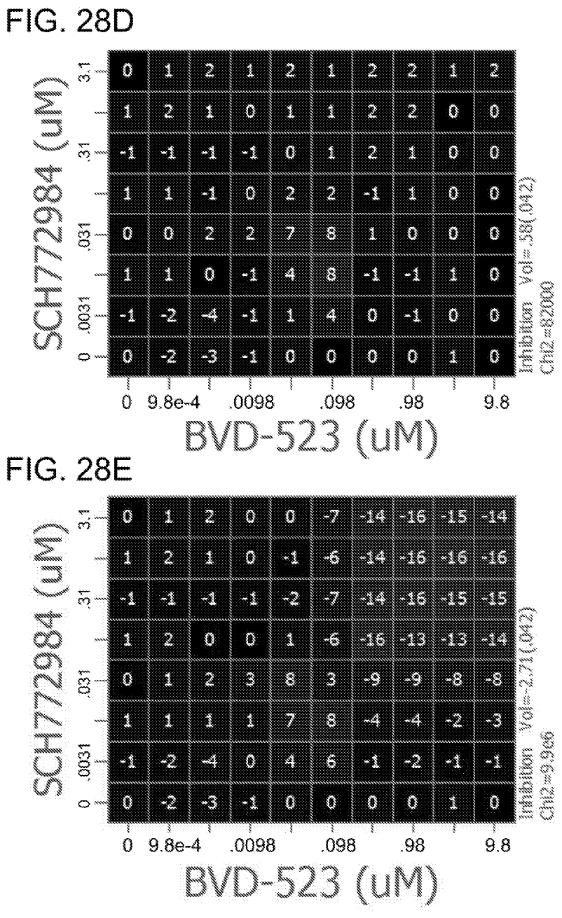

Visualization of the Loewe and Bliss ‘excess inhibition’ heat maps suggested that the combination of BVD-523 and SCH772984 was mainly additive with windows of potential synergy in mid-range doses (FIG. 28A-FIG. 28E).

In summary, these results suggest that interactions between BVD-523 and SCH772984 are at least additive, and in some cases synergistic.

### Example 9

Targeting the MAPK Signaling Pathway in Cancer: Promising Activity with the Novel Selective ERK1/2 Inhibitor BVD-523 (Ulixertinib)

Treatment strategies for cancer have evolved from classic cytotoxic-based approaches to agents that counteract the effects of genetic lesions that drive aberrant signaling essential to tumor proliferation and survival. For example, the ERK module of the mitogen-activated protein kinase (MAPK) signaling cascade (RAS-RAF-MEK-ERK) (Cargnello and Rouxx 2011) can be engaged by several receptor tyrosine kinases (e.g., EGFR and ErbB-2) in addition to constitutively activated mutations of pathway components such as RAS and BRAF (Gollob et al. 2006). Through aberrant activation of ERK signaling, genetic alterations in RAS or BRAF result in rapid tumor growth, increased cell survival, and resistance to apoptosis (Poulikakos et al. 2011, Corcoran et al. 2010, Nazarian et al. 2010, Shi et al. 2014, Wagle et al. 2011). Activating mutations of RAS family members KRAS and NRAS are found in ~30% of all human cancers, with particularly high incidence in pancreatic (Kanda et al. 2012) and colorectal cancer (Arrington et al. 2014). Constitutively activating mutations in the BRAF gene that normally encodes for valine at amino acid 600 have been observed in melanoma, thyroid carcinoma, colorectal cancer, and non-small cell lung cancer (Hall et al. 2014). Cancers bearing genetic mutations that result in changes of the downstream components ERK and MEK have also been reported (Ojesina et al. 2014, Arcila et al. 2015). Alterations that activate the MAPK pathway are also common in the setting of resistance to targeted therapies (Groenendijk et al. 2014). Thus, targeting the MAPK pathway terminal master kinases (ERK1/2) is a promising strategy for tumors harboring such pathway activating alterations (e.g., BRAF, NRAS, and KRAS).

Three MAPK pathway-targeting drugs have been approved by the US Food and Drug Administration (FDA) for single-agent treatment of nonresectable or metastatic cutaneous melanoma with BRAFV600 mutations: the BRAF inhibitors vemurafenib and dabrafenib and the MEK inhibitor trametinib. Furthermore, the combination of dabrafenib and trametinib is also approved in this indication (Queirolo et al. 2015 and Massey et al. 2015). An additional MEK inhibitor, cobimetinib, is approved in this indication as part of a combination regimen with BRAF inhibitors. Clinical experience with these drugs validates the MAPK pathway as a therapeutic target. In phase III trials of patients with BRAFV600-mutant melanoma, the single agents vemurafenib and dabrafenib demonstrated superior response rates (approximately 50% vs. 5-19%) and median progression-free survival (PFS, 5.1-5.3 months vs. 1.6-2.7 months) over cytotoxic chemotherapy (dacarbazine) (Chapman et al. 2011 and Hauschild et al. 2012). Furthermore, clinical use of concomitant BRAF-plus MEK-targeted therapies has demonstrated that simultaneous targeting of different nodes in the MAPK pathway can enhance the magnitude and duration of response. First-line use of BRAF plus MEK-targeted agents (dabrafenib/trametinib or cobimetinib/vemurafenib) further improved median overall survival compared with single-agent BRAF inhibition (Robert et al. 2015, Long et al. 2015, Larkin et al. 2014). Thus, combined BRAF-/MEK-targeted therapy is a valuable treatment option for patients with metastatic melanoma with BRAFV600 mutations.

Despite improvements in clinical outcomes seen with BRAF-/MEK-inhibitor combination therapies, durable benefit is limited by the eventual development of acquired resistance and subsequent disease progression, with median PFS ranging from approximately 9 to 11 months. (Robert et al. 2015, Long et al. 2015, Larkin et al. 2014, and Flaherty et al. 2012). Genetic mechanisms of acquired resistance to single-agent BRAF inhibition have been intensely studied, and identification of resistance mechanisms include splice variants of BRAF (Poulikakos et al. 2011), BRAFV600E amplification (Corcoran et al. 2010), MEK mutations (Wagle et al. 2014), NRAS mutations, and RTK activation (Nazarian et al. 2010 and Shi et al. 2014). Resistance mechanisms in the setting of BRAF-/MEK-inhibitor combination therapy are beginning to emerge and mirror that of BRAF single-agent resistance (Wagle et al. 2014 and Long et al. 2014). These genetic events all share in common the ability to reactivate ERK signaling. Indeed, reactivated MAPK pathway signaling as measured by ERK transcriptional targets is common in tumor biopsies from BRAF inhibitor-resistant patients (Rizos et al. 2014). Furthermore, ERK1/2 reactivation has been observed in the absence of a genetic mechanism of resistance (Carlino et al. 2015). Therefore, the quest to achieve durable clinical benefit has led researchers to focus on evaluating additional agents that target the downstream MAPK components ERK1/2. Inhibiting ERK may provide important clinical benefit to patients with acquired resistance to BRAF/MEK inhibition. ERK family kinases have shown promise as therapeutic targets in preclinical cancer models, including those cancers resistant to BRAF or MEK inhibitors (Morris et al. 2013 and Hatzivassiliou et al. 2012). However, the potential use of such ERK1/2 inhibitors expands beyond acquired-resistance in melanoma.

Targeting ERK1/2 is a rational strategy in any tumor type harboring known drivers of MAPK, not only BRAF/MEK therapy-relapsed patients. As ERK1 and ERK2 reside downstream in the pathway, they represent a particularly attractive treatment strategy within the MAPK cascade that may avoid upstream resistance mechanisms. Here, preclinical characterization of BVD-523 (ulixertinib) in models of MAPK pathway-dependent cancers is reported, including drug-naïve and BRAF/MEK therapy acquired-resistant models. Results of a phase I dose-finding study of BVD-523 are included as a companion publication in this journal. See, Examples 17-24.

In the present invention, BVD-523 was shown to be a potent, highly selective, reversible, small molecule ATP-competitive inhibitor of ERK1/2 with in vitro and in vivo anticancer activity.

BVD-523 (ulixertinib) was identified and characterized as a novel, reversible, ATP-competitive ERK1/2 inhibitor with high potency and ERK1/2 selectivity. BVD-523 caused reduced proliferation and enhanced caspase activity, most notably in cells harboring MAPK (RAS-RAF-MEK) pathway mutations. In in vivo BRAFV600E xenograft studies, BVD-523 showed dose-dependent growth inhibition and tumor regressions. Interestingly, BVD-523 inhibited phosphorylation of target substrates despite increased phosphorylation of ERK1/2. BVD-523 also demonstrated antitumor activity in models of acquired resistance to single-agent and combination BRAF/MEK targeted therapy. Synergistic antiproliferative effects in a BRAF^{V600E}-mutant melanoma cell line xenograph model were also demonstrated when BVD-523 was used in combination with BRAF inhibition. These studies suggest that BVD-523 holds promise as a treatment for ERK-dependent cancers, including those whose tumors have acquired resistance to other treatments targeting upstream nodes of the MAPK pathway.

### Example 10

### Discovery and Initial Characterization of a Novel ERK1/2 Inhibitor, BVD-523 (Ulixertinib)

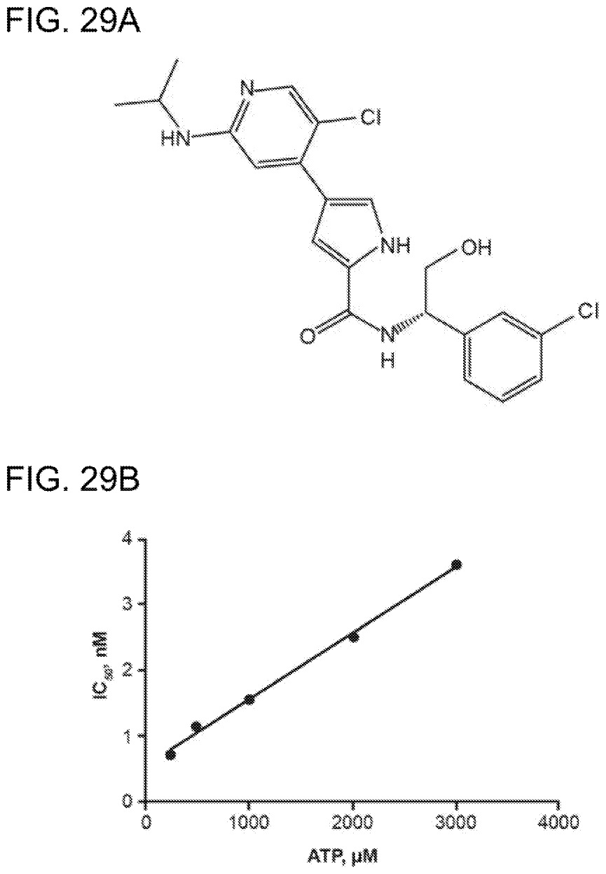

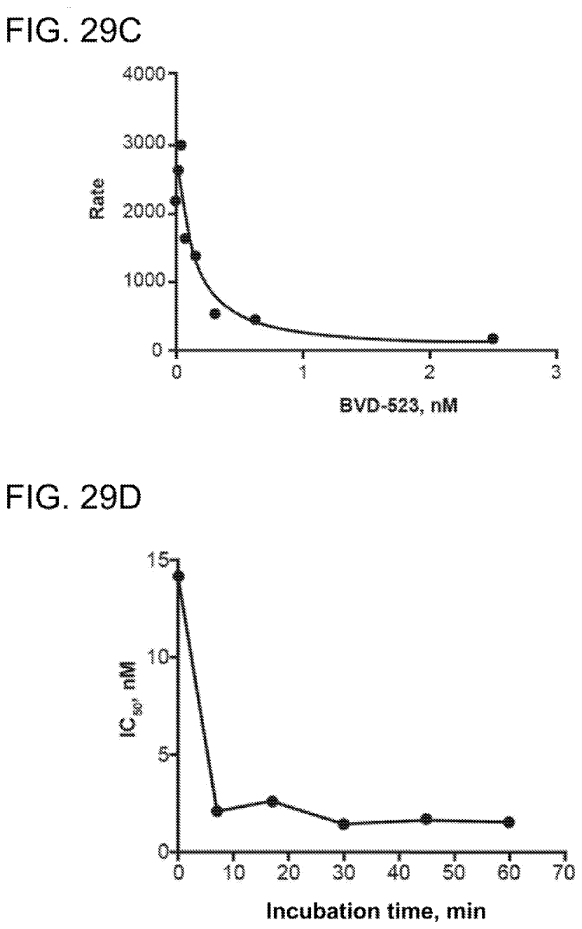

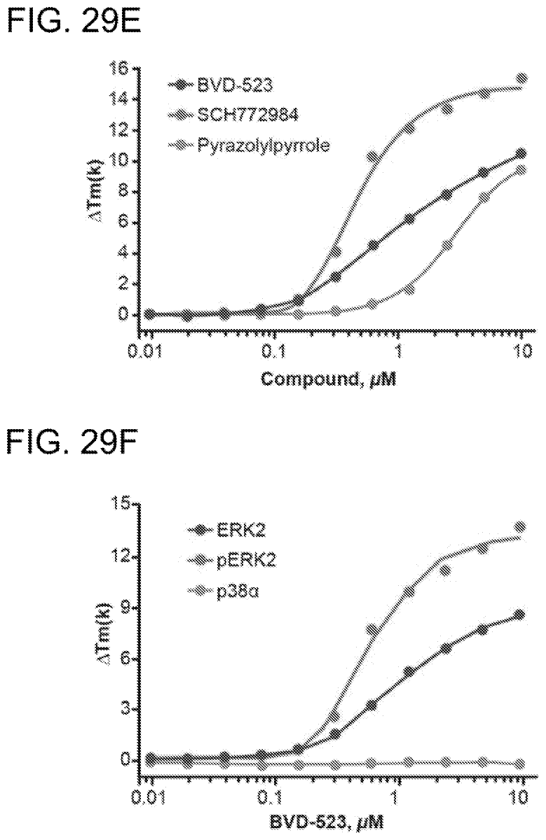

Following extensive optimization of leads originally identified using a high-throughput, small-molecule screen (Aronov et al. 2009), a novel adenosine triphosphate (ATP)-competitive ERK1/2 inhibitor, BVD-523 (ulixertinib) was identified (FIG. 29A). BVD-523 is a potent ERK inhibitor with a Ki of 0.04±0.02 nM against ERK2. It was shown to be a reversible, competitive inhibitor of ATP, as the IC₅₀ values for ERK2 inhibition increased linearly with increasing ATP concentration (FIG. 29B and FIG. 29C). The IC₅₀ remained nearly constant for incubation times ≥10 minutes, suggesting rapid equilibrium and binding of BVD-523 with ERK2 (FIG. 29D). BVD-523 is also a tight-binding inhibitor of recombinant ERK1 (Rudolph et al. 2015), exhibiting a Kᵢ of <0.3 nM.

Binding of BVD-523 to ERK2 was demonstrated using calorimetric studies and compared to data generated using the ERK inhibitors SCH772984 and pyrazolylpyrrole (Arovov et al. 2007). All compounds bound and stabilized inactive ERK2 with increasing concentration, as indicated by positive ΔTm values (FIG. 29E). The 10- to 15-degree change in ΔTm observed with BVD-523 and SCH-772984 is consistent with compounds that have low-nanomolar binding affinities (Fedorov et al. 2012). BVD-523 demonstrated a strong binding affinity to both phosphorylated active ERK2 (pERK2) and inactive ERK2 (FIG. 29F). A stronger affinity to pERK2 compared with inactive ERK2 was observed. BVD-523 did not interact with the negative control protein p38a MAP kinase (FIG. 29F).

BVD-523 demonstrated excellent ERK1/2 kinase selectivity based on biochemical counter-screens against 75 kinases in addition to ERK1 and ERK2. The ATP concentrations were approximately equal to the Kₘ in all assays. Kinases inhibited to greater than 50% by 2 μM BVD-523 were retested to generate Kᵢ values (or apparent Ki; Table 21). Twelve of the 14 kinases had a Kᵢ of <1 μM. The selectivity of BVD-523 for ERK2 was >7000-fold for all kinases tested except ERK1, which was inhibited with a Ki of <0.3 nM (10-fold). Therefore, BVD-523 is a highly potent and selective inhibitor of ERK1/2.

TABLE 21
BVD-523 displays selectivity for ERK1 and
ERK2 kinases.

| Kinase | Ki (μM) |
|---|---|
| CDK1/cyclinB | 0.07^{a} |
| CDK2/cyclinA | 0.36 |
| CDK5/p35 | 0.09^{a} |
| CDK6/cycinD3 | 0.09 |
| ERK1 | 0.0003 |
| ERK2 | 0.00004 |
| GSK3b | 0.32 |
| JNK2α | 0.65^{a} |
| JNIK3 | 1.3 |
| P38γ | 0.45^{a} |
| P38δ | 0.24 |
| ROCKI | 11.1 |
| ROCKII | 0.27^{a} |
| RSK3 | 0.45 |

^{a}Apparent.
<50% inhibition at 2μM: ABL, AKT3, AMPK, AUR1, AUR2, AXL, BLX, CAMKII, CAMKIV, CHK1, CHK2, CK1, CK2, CSK, EGFR, EPHB4, FES, FGFR3, FLT3, FYN, IGF1R, IKKα, 1KKβ, IKKi, IRAK4, IRTK, 1TK, JAK3, JNKlα 1, KDR, LCK, LYN, cMET, MKK4, MKK6, MKK7β, MLK2, MSK1, MST2, NAK, NEK2, p38α, p38β, p70S6K, PAK2, PDGFRα, PDKI, PKA, PKQα, pKCβ II, PKCγ, PKCi, PKCθ, PRAK, PRK2, cRAF, SGK, SRC, SYK, TAM, TiE2, ZAP70

### Example 11

BVD-523 Preferentially Inhibits Cellular Proliferation and Enhances Caspase-3/7 Activity In Vitro in Cancer Cell Lines with MAPK Pathway-Activating Mutations

BVD-523 cellular activity was assessed in a panel of approximately 1,000 cancer cell lines of various lineages and genetic backgrounds (FIG. 30A and Table 22). Cell lines were classified as MAPK wild type (wt) or mutant depending on the absence or presence of mutations in RAS family members and BRAF. Although some MAPK-wt cell lines were sensitive to BVD-523, generally BVD-523 inhibited proliferation preferentially in cells with MAPK pathway alterations.

Next, the growth and survival impact of BVD-523 treatment on sensitive cells was characterized. Fluorescence activated cell sorting (FACS) analysis was performed on BRAF^{V600E}-mutant melanoma cell line UACC-62 following treatment with BVD-523 at 500 nM or 2000 nM for 24 hours. Treated cells were arrested in the G1 phase of the cell cycle in a concentration-dependent manner (FIG. 30B).

In addition, caspase-3/7 activity was analyzed as a measure of apoptosis in multiple human cancer cell lines. A concentration- and cell-line-dependent increase in caspase 3/7 was observed following treatment with BVD-523 for 72 hours (FIG. 30C). BVD-523 treatment resulted in pronounced caspase-3/7 induction in a subset of MAPK-activated cell lines harboring a BRAFV600 mutation (A375, WM266, and LS411N). This is consistent with earlier observations for preferential inhibition of proliferation by BVD-523 in MAPK pathway-mutant cancer cell lines (FIG. 30A).

To further characterize the mechanism of action and effects on signaling elicited by BVD-523, the levels of various effector and MAPK-related proteins were assessed in BVD-523-treated BRAF^{V600E}-mutant A375 melanoma cells (FIG. 30D). Phospho-ERK1/2 levels increased in a concentration-dependent manner after 4 and 24 hours of BVD-523 treatment. Despite prominent concentration-dependent increases in pERK1/2 observed with 2 μM BVD-523 treatment, phosphorylation of the ERK1/2 target RSK1/2 was reduced at both 4 and 24 hours, which is consistent with sustained inhibition. Total protein levels of DUSP6, a distal marker of ERK1/2 activity, were also attenuated at 4 and 24 hours. Following 24 hours of treatment with BVD-523, the apoptotic marker BIM-EL increased in a dose-dependent manner, while cyclin D-1 and pRB was attenuated at 2 μM. All effects are consistent with on-target ERK1/2 inhibition.

TABLE 22

| | | Cell | | | Fitted | Viability ratio | | | | | | | | |
|---|---|---|---|---|---|---|---|---|---|---|---|---|---|---|
| Barcode | Organ | ID | Cell Line | Compound No | MGH.IC50 | 20 uM | 10 uM | 5 uM | 2.5 uM | 1.25 uM | 0.625 uM | 0.3125 uM | 0.15625 uM | 0.078125 uM |
| 026_8049_00277140 | Biliary Tract | 8049 | ETK-1 | 456 | 3.525905 | 0.587 | 0.805 | 0.874 | 0.9304 | 0.8796 | 0.954 | 1.0285 | 1.094 | 0.9918 |
| 026_664_00277150 | Biliary Tract | 664 | HuCCT1 | 456 | 3.600435 | 0.663 | 0.734 | 0.847 | 0.9661 | 0.9652 | 1.029 | 0.9656 | 1.0172 | 0.9981 |
| 026_653_00278500 | Biliary Tract | 653 | EGI-1 | 456 | 4.229085 | 0.693 | 0.686 | 0.74 | 0.7843 | 0.8546 | 0.889 | 0.967 | 0.9286 | 0.9525 |
| 026_8204_00278540 | Biliary Tract | 8204 | TGBC24TKB | 456 | 5.609877 | 0.768 | 0.875 | 0.826 | 0.8122 | 0.8629 | 0.862 | 0.8909 | 0.9353 | 0.9353 |
| 026_8188_00293390 | Biliary Tract | 8188 | TGBC1TKB | 456 | 6.179372 | 0.915 | 0.929 | 0.913 | 0.9808 | 0.9201 | 1.144 | 1.0128 | 0.9048 | 0.9496 |
| 026_330_00278580 | Bone | 330 | H-EMC-SS | 456 | 0.038629 | 0.36 | 0.392 | 0.383 | 0.4615 | 0.437 | 0.566 | 0.7772 | 0.9442 | 0.9662 |
| 026_8047_00283120 | Bone | 8047 | ES7 | 456 | 1.846677 | 0.515 | 0.521 | 0.539 | 0.5362 | 0.7569 | 0.769 | 0.8353 | 0.9371 | 0.9503 |
| 026_8053_00287650 | Bone | 8053 | EW-13 | 456 | 2.197657 | 0.333 | 0.545 | 0.665 | 0.8165 | 0.9099 | 0.962 | 0.9571 | 0.9945 | 1.0406 |
| 026_8227_00288230 | Bone | 8227 | CADO-ES1 | 456 | 2.29467 | 0.359 | 0.554 | 0.585 | 1.0389 | 0.9034 | 0.952 | 1.1264 | 1.2671 | 1.0023 |
| 026_8050_00279380 | Bone | 8050 | EW-1 | 456 | 2.409222 | 0.487 | 0.555 | 0.593 | 0.6879 | 0.6908 | 0.788 | 0.8057 | 0.892 | 0.9331 |
| 026_306_00278530 | Bone | 306 | SK-ES-1 | 456 | 2.4607 | 0.527 | 0.556 | 0.577 | 0.682 | 0.673 | 0.855 | 0.8706 | 0.8641 | 0.8867 |
| 026_305_00277180 | Bone | 305 | U-2 OS | 456 | 2.847932 | 0.145 | 0.599 | 0.657 | 0.7441 | 0.8231 | 0.793 | 0.8449 | 0.9969 | 0.8925 |
| 026_337_00283440 | Bone | 337 | HuO9 | 456 | 2.916396 | 0.448 | 0.696 | 0.939 | 0.8026 | 0.8478 | 0.903 | 0.9796 | 0.8682 | 1.0954 |
| 26_8227_00304340 | Bone | 8227 | CADO-ES1 | 456 | 2.975471 | 0.542 | 0.666 | 0.884 | 0.8971 | 0.9918 | 0.974 | 1.0247 | 1.0031 | 1.0728 |
| 026_8043_00283110 | Bone | 8043 | ES1 | 456 | 2.981717 | 0.543 | 0.663 | 0.705 | 0.7865 | 0.811 | 0.835 | 0.7995 | 0.9278 | 0.8292 |
| 026_8142_00282550 | Bone | 8142 | NOS-1 | 456 | 3.203574 | 0.547 | 0.775 | 0.825 | 0.892 | 0.7556 | 0.894 | 0.8477 | 1.1038 | 0.9881 |
| 026_8055_00290580 | Bone | 8055 | EW-18 | 456 | 3.311765 | 0.638 | 0.688 | 0.718 | 1.1012 | 0.9293 | 0.993 | 1.0646 | 1.1078 | 1.0895 |
| 026_8058_00293350 | Bone | 8058 | EW-3 | 456 | 3.365484 | 0.715 | 0.595 | 0.72 | 0.7195 | 0.8774 | 0.8 | 0.8628 | 0.9101 | 1.2148 |
| 026_339_00277160 | Bone | 339 | NY | 456 | 3.400937 | 0.59 | 0.789 | 0.875 | 0.9222 | 0.9594 | 0.975 | 0.933 | 1.1433 | 0.9637 |
| 026_8165_00287690 | Bone | 8165 | SK-PN-DW | 456 | 3.476926 | 0.621 | 0.812 | 0.933 | 0.9706 | 0.9188 | 0.991 | 1.0026 | 1.0012 | 1.0432 |
| 026_326_00282540 | Bone | 326 | MHH-ES-1 | 456 | 3.524605 | 0.618 | 0.719 | 0.824 | 0.8235 | 0.8745 | 0.966 | 1.1187 | 1.0416 | 0.913 |
| 026_8048_00279370 | Bone | 8048 | ES8 | 456 | 3.530755 | 0.572 | 0.698 | 0.765 | 0.8053 | 0.8255 | 0.912 | 0.931 | 0.9069 | 0.9095 |
| 026_331_00278590 | Bone | 331 | HOS | 456 | 3.602447 | 0.53 | 0.798 | 0.749 | 0.7609 | 0.8727 | 0.839 | 0.8786 | 0.8236 | 0.972 |
| 026_8045_00282660 | Bone | 8045 | ES5 | 456 | 3.665641 | 0.704 | 0.653 | 0.874 | 0.8827 | 0.7407 | 0.913 | 0.8294 | 1.0585 | 1.162 |
| 026_8059_00283090 | Bone | 8059 | EW-7 | 456 | 3.82717 | 0.662 | 0.72 | 0.76 | 0.831 | 0.8857 | 0.95 | 0.9309 | 0.9266 | 0.9963 |
| 026_8201_00282520 | Bone | 8201 | ES3 | 456 | 3.982944 | 0.707 | 0.721 | 0.778 | 0.8414 | 0.8385 | 0.989 | 0.9588 | 1.0248 | 1.0261 |
| 026_8056_00314310 | Bone | 8056 | EW-22 | 456 | 4.196343 | 0.701 | 0.827 | 0.819 | 0.8824 | 0.9413 | 0.894 | 0.9848 | 1.0221 | 1.1139 |
| 026_329_00282700 | Bone | 329 | G-292 Clone | 456 | 4.264519 | 0.705 | 0.918 | 0.893 | 0.949 | 1.0435 | 1.016 | 0.9814 | 0.9215 | 1.1209 |
| | | | A141B1 | | | | | | | | | | | |
| 026_324_00278550 | Bone | 324 | CAL-72 | 456 | 4.286956 | 0.731 | 0.942 | 0.942 | 0.9262 | 0.9863 | 0.966 | 0.9797 | 0.9547 | 1.0113 |
| 026_304_00283460 | Bone | 304 | Saos-2 | 456 | 4.597501 | 0.742 | 0.784 | 0.9 | 0.8765 | 0.9306 | 0.926 | 0.9516 | 0.95 | 1.0352 |
| 026_325_00283060 | Bone | 325 | CAL-78 | 456 | 4.650689 | 0.727 | 0.913 | 0.918 | 0.9149 | 0.8999 | 0.897 | 0.9065 | 1.0242 | 1.0181 |
| 026_1138_00278560 | Bone | 1138 | CS1 | 456 | 4.765346 | 0.744 | 0.848 | 0.864 | 0.8601 | 0.8818 | 1.016 | 1.0005 | 0.9749 | 0.977 |
| 026_8162_00282560 | Bone | 8162 | SJSA-1 | 456 | 4.867747 | 0.802 | 0.803 | 0.805 | 0.7765 | 0.762 | 0.974 | 1.0651 | 1.0412 | 1.0623 |
| 026_336_00283430 | Bone | 336 | HuO-3N1 | 456 | 4.900372 | 0.724 | 0.86 | 0.806 | 0.8427 | 0.8308 | 0.87 | 0.8972 | 0.9772 | 1.0073 |
| 026_328_00278600 | Bone | 328 | TC-71 | 456 | 5.117725 | 0.844 | 0.939 | 0.97 | 1.0221 | 0.9857 | 1.072 | 1.023 | 1.0842 | 1.0771 |
| 026_8054_00282530 | Bone | 8054 | EW-16 | 456 | 5.180986 | 0.835 | 0.775 | 0.925 | 0.8021 | 0.8155 | 0.934 | 0.9339 | 1.058 | 1.1414 |
| 026_335_00308220 | Bone | 3355 | MG-63 | 456 | 5.257203 | 0.884 | 0.841 | 0.852 | 0.8745 | 1.0185 | 1.081 | 1.1007 | 1.0325 | 1.0709 |
| 026_1241_00283070 | Bone | 1241 | CHSA8926 | 456 | 5.394244 | 0.883 | 0.851 | 0.947 | 0.9788 | 0.9327 | 1.019 | 1.0879 | 1.0294 | 1.119 |
| 026_8044_00279340 | Bone | 8044 | ES4 | 456 | 6.158016 | 0.822 | 0.876 | 0.92 | 0.8612 | 0.8592 | 0.873 | 0.8969 | 0.9914 | 0.9632 |
| 026_8057_00283080 | Bone | 8057 | EW-24 | 456 | 6.273162 | 0.927 | 0.899 | 0.97 | 0.9817 | 0.9704 | 1.012 | 0.9822 | 0.9596 | 0.9793 |
| 026_8051_00285230 | Bone | 8051 | EW-11 | 456 | 6.340509 | 0.924 | 1.008 | 0.846 | 0.9309 | 1.047 | 0.868 | 0.9762 | 1.0834 | 1.048 |
| 026_8046_00279351 | Bone | 8046 | ES6 | 456 | 6.745328 | 0.882 | 1.065 | 1.046 | 1.0203 | 1.0617 | 0.85 | 0.8881 | 1.0547 | 0.9578 |
| 026_8146_00285140 | Brain | 8146 | ONS-76 | 456 | 1.017095 | 0.362 | 0.369 | 0.387 | 0.52 | 0.6146 | 0.733 | 0.8773 | 0.9176 | 1.1016 |
| 026_8009_00285111 | Brain | 8009 | AM-38 | 456 | 2.859683 | 0.545 | 0.617 | 0.664 | 0.5881 | 0.5592 | 0.692 | 0.826 | 0.9522 | 1.078 |
| 026_8091_00285281 | Brain | 8091 | KS-1 | 456 | 2.979142 | 0.617 | 0.59 | 0.62 | 0.7057 | 0.7844 | 0.875 | 0.9025 | 1.0584 | 1.1343 |
| 026_388_00285240 | Brain | 388 | MOG-G-CCM | 456 | 3.029922 | 0.505 | 0.754 | 0.852 | 0.977 | 0.9597 | 0.989 | 1.1211 | 1.0361 | 1.0747 |
| 026_352_00283150 | Brain | 352 | LN-229 | 456 | 3.053877 | 0.552 | 0.647 | 0.662 | 0.7206 | 0.8119 | 0.932 | 0.9285 | 1.0381 | 1.0748 |
| 026_8214_00290680 | Brain | 8214 | YH-13 | 456 | 3.068585 | 0.558 | 0.628 | 0.773 | 1.0443 | 0.9614 | 1.113 | 1.1139 | 1.093 | 1.2619 |
| 026_8214_00288290 | Brain | 8214 | YH-13 | 456 | 3.531592 | 0.627 | 0.651 | 0.866 | 0.8387 | 0.8073 | 0.922 | 1.077 | 1.0619 | 1.1182 |
| 026_358_00293700 | Brain | 358 | D283 Med | 456 | 3.551231 | 0.64 | 0.743 | 0.916 | 0.9078 | 0.9464 | 0.928 | 0.871 | 0.9767 | 1.4204 |
| 026_8061_00290830 | Brain | 8061 | GB-1 | 456 | 3.686496 | 0.613 | 0.692 | 0.809 | 0.8255 | 0.8977 | 0.877 | 0.9922 | 0.9416 | 0.8923 |
| 026_374_00283180 | Brain | 374 | U-251MG | 456 | 3.933399 | 0.654 | 0.733 | 0.861 | 0.881 | 0.8991 | 0.822 | 0.926 | 0.9972 | 1.1342 |
| 026_343_00283160 | Brain | 343 | PFSK-1 | 456 | 3.963833 | 0.626 | 0.904 | 1.007 | 129.02 | 0.934 | 1.078 | 1.0411 | 1.0084 | 1.052 |
| 026_393_00283190 | Brain | 393 | YKG-1 | 456 | 3.987729 | 0.646 | 0.714 | 0.77 | 0.8178 | 0.8374 | 0.848 | 0.953 | 0.8805 | 1.0209 |
| 026_8028_00287630 | Brain | 8028 | D-263MG | 456 | 4.157483 | 0.662 | 0.709 | 0.734 | 0.7813 | 0.8341 | 0.839 | 0.9023 | 0.9333 | 1.0435 |
| 026_379_00283140 | Brain | 379 | GAMG | 456 | 4.214093 | 0.681 | 0.701 | 0.829 | 0.8043 | 0.8412 | 0.83 | 0.9322 | 0.8908 | 1.046 |
| 026_8019_00293320 | Brain | 8019 | CAS-1 | 456 | 4.309856 | 0.732 | 0.772 | 0.825 | 0.9222 | 0.9783 | 1.227 | 0.8865 | 0.8407 | 1.1628 |
| 026_8001_00285100 | Brain | 8001 | 8-MG-BA | 456 | 4.345495 | 0.688 | 0.845 | 0.88 | 0.8958 | 0.9285 | 0.912 | 0.9148 | 0.9398 | 1.07 |
| 026_351_00283450 | Brain | 351 | LN-18 | 456 | 4.481815 | 0.685 | 0.822 | 0.862 | 0.8746 | 0.8966 | 0.902 | 0.9022 | 0.9311 | 0.9325 |
| 026_357_00283410 | Brain | 357 | H4 | 456 | 4.481909 | 0.721 | 0.808 | 0.876 | 0.858 | 0.8822 | 0.94 | 1.0232 | 0.9287 | 1.0614 |
| 026_8085_00293731 | Brain | 8085 | KINGS-1 | 456 | 4.48888 | 0.797 | 0.781 | 0.82 | 0.9645 | 0.9641 | 1.015 | 0.9981 | 1.0442 | 0.974 |
| 026_350_00284910 | Brain | 350 | M059J | 456 | 4.537622 | 0.723 | 0.834 | 0.869 | 0.8888 | 0.9473 | 0.909 | 0.9144 | 1.0191 | 1.0818 |
| 026_8015_00308070 | Brain | 8015 | Becker | 456 | 4.548091 | 0.696 | 0.814 | 0.876 | 0.8121 | 0.825 | 0.889 | 0.917 | 0.9113 | 1.0138 |
| 026_8160_00287680 | Brain | 8160 | SF539 | 456 | 4.738405 | 0.726 | 0.919 | 0.827 | 0.8875 | 0.9392 | 0.934 | 0.952 | 1.0525 | 0.9285 |
| 026_8159_00287670 | Brain | 8159 | SF268 | 456 | 4.804304 | 0.786 | 0.825 | 0.923 | 0.9225 | 0.9772 | 0.99 | 0.978 | 0.9754 | 0.9699 |
| 026_359_00283100 | Brain | 359 | Daoy | 456 | 4.81575 | 0.683 | 0.74 | 0.796 | 0.8102 | 0.876 | 0.896 | 0.9135 | 0.9254 | 0.9257 |
| 026_8217_00290890 | Brain | 8217 | SK-MG-1 | 456 | 4.83361 | 0.725 | 0.784 | 0.83 | 0.844 | 0.8849 | 0.901 | 0.9119 | 0.9353 | 0.9859 |
| 026_342_00285160 | Brain | 342 | SW 1783 | 456 | 4.84471 | 0.796 | 0.905 | 0.896 | 0.9349 | 1.0662 | 0.935 | 1.0347 | 1.0148 | 1.08 |
| 026_8029_00288240 | Brain | 8029 | D-336MG | 456 | 4.925588 | 0.792 | 0.892 | 0.997 | 0.8872 | 0.9556 | 0.944 | 1.105 | 1.0724 | 1.1057 |
| 026_8030_00295500 | Brain | 8030 | D-392MG | 456 | 4.966575 | 0.791 | 0.866 | 0.824 | 0.9331 | 0.8638 | 1.022 | 0.9531 | 0.9688 | 1.0531 |
| 026_8089_00287440 | Brain | 8089 | KNS-81-FD | 456 | 5.077406 | 0.804 | 0.89 | 0.869 | 0.8138 | 0.8421 | 1.049 | 1.0747 | 0.9317 | 1.1222 |
| 026_8138_00285290 | Brain | 8138 | NMC-G1 | 456 | 5.086457 | 0.771 | 0.817 | 0.763 | 0.762 | 0.8138 | 0.887 | 0.968 | 1.0104 | 1.049 |
| 026_8139_00285130 | Brain | 8139 | no-10 | 456 | 5.147267 | 0.824 | 0.888 | 0.928 | 0.9069 | 0.9436 | 1.031 | 0.943 | 1.05 | 1.0543 |
| 026_8083_00293720 | Brain | 8083 | KALS-1 | 456 | 5.203248 | 0.817 | 0.765 | 0.902 | 0.9843 | 0.9376 | 0.915 | 0.9382 | 1.0116 | 0.9496 |
| 026_378_00284880 | Brain | 378 | DK-MG | 456 | 5.2953 | 0.789 | 0.757 | 0.713 | 0.7179 | 0.7297 | 0.849 | 0.8425 | 0.9101 | 1.0114 |
| 026_383_00284900 | Brain | 383 | LN-405 | 456 | 5.313289 | 0.839 | 0.886 | 0.901 | 0.937 | 0.8868 | 1.041 | 0.949 | 1.0816 | 1.0986 |
| 026_8032_00293340 | Brain | 8032 | D-542MG | 456 | 5.342096 | 0.822 | 0.856 | 0.927 | 0.9108 | 0.8977 | 0.934 | 0.9191 | 0.9743 | 0.9895 |
| 026_344_00282720 | Brain | 344 | LNZTA3WT4 | 456 | 5.43481 | 0.777 | 1.136 | 0.931 | 0.9386 | 0.9371 | 1.018 | 1.0441 | 1.0001 | 0.9944 |
| 026_8167_00290910 | Brain | 8167 | SNB75 | 456 | 5.474524 | 0.76 | 0.772 | 0.83 | 0.8023 | 0.8147 | 1.01 | 0.8804 | 1.0523 | 0.9382 |
| 026_8087_00285270 | Brain | 8087 | KNS-42 | 456 | 5.484622 | 0.862 | 0.858 | 0.846 | 0.8777 | 0.9301 | 0.972 | 0.9923 | 1.0701 | 1.0135 |
| 026_354_00287481 | Brain | 354 | U-87MG | 456 | 5.588679 | 0.851 | 0.949 | 0.942 | 0.9605 | 0.9939 | 1.004 | 1.0222 | 0.9964 | 1.0855 |
| 026_8140_00285300 | Brain | 8140 | no-11 | 456 | 5.608459 | 0.844 | 0.885 | 0.896 | 0.959 | 0.9411 | 0.893 | 0.9627 | 1.0352 | 0.9432 |
| 026_8221_00284860 | Brain | 8221 | D-423MG | 456 | 5.731372 | 0.807 | 0.783 | 0.798 | 0.8469 | 0.8833 | 0.945 | 0.9877 | 0.9131 | 0.9215 |
| 026_348_00283400 | Brain | 348 | DBTRG-05MG | 456 | 5.749405 | 0.792 | 0.75 | 0.763 | 0.7919 | 0.7917 | 0.856 | 0.9393 | 0.9013 | 0.9922 |
| 026_341_00285310 | Brain | 341 | SW 1088 | 456 | 5.805148 | 0.869 | 0.907 | 0.895 | 0.8893 | 0.9105 | 0.919 | 0.966 | 0.8961 | 1.0957 |
| 026_356_00283420 | Brain | 356 | Hs 683 | 456 | 5.858982 | 0.841 | 0.993 | 0.888 | 0.8789 | 0.8908 | 1.04 | 0.8743 | 0.906 | 1.058 |
| 026_8031_00287640 | Brain | 8031 | D-502MG | 456 | 5.99897 | 0.825 | 0.819 | 0.771 | 0.7649 | 0.8658 | 0.834 | 0.9457 | 0.9922 | 0.9572 |
| 026_8224_00284870 | Brain | 8224 | D-566MG | 456 | 6.026403 | 0.839 | 0.84 | 0.858 | 0.8841 | 0.9252 | 0.941 | 0.968 | 0.9815 | 0.9816 |
| 026_389_00284920 | Brain | 389 | MOG-G-UVW | 456 | 6.074777 | 0.861 | 0.876 | 0.879 | 0.9129 | 0.9004 | 0.902 | 1.0597 | 0.9108 | 1.0304 |
| 026_341_00283470 | Brain | 341 | SW 1088 | 456 | 6.105387 | 0.913 | 0.89 | 0.897 | 0.897 | 0.9463 | 0.931 | 0.9486 | 0.9506 | 1.1024 |
| 026_375_00284850 | Brain | 375 | 42-MG-BA | 456 | 6.106423 | 0.896 | 0.892 | 0.932 | 0.944 | 0.9633 | 0.976 | 0.9735 | 0.9871 | 1.0285 |
| 026_1122_00283170 | Brain | 1122 | SF-295 | 456 | 6.112956 | 0.879 | 0.909 | 0.909 | 0.9301 | 0.9215 | 0.946 | 0.9199 | 0.9433 | 1.0329 |
| 026_8158_00290650 | Brain | 8158 | SF126 | 456 | 6.158755 | 0.86 | 0.97 | 1 | 1.0676 | 1.0025 | 1.187 | 0.9851 | 0.9627 | 1.1324 |
| 026_340_00285250 | Brain | 340 | CCF-STTG1 | 456 | 6.170298 | 0.851 | 0.911 | 0.916 | 0.9011 | 0.9748 | 0.919 | 0.9109 | 0.9323 | 0.9837 |
| 026_380_00284890 | Brain | 380 | GMS-10 | 456 | 6.23472 | 0.842 | 0.885 | 0.853 | 0.8466 | 0.8914 | 0.914 | 0.8882 | 1.0019 | 1.0545 |
| 026_354_00290361 | Brain | 354 | U-87 MG | 456 | 6.315816 | 0.93 | 0.874 | 0.977 | 0.9346 | 0.9336 | 1.084 | 0.9295 | 1.062 | 1.073 |
| 026_8063_00290841 | Brain | 8063 | GI-1 | 456 | 6.443002 | 0.809 | 0.877 | 0.913 | 0.9018 | 0.9184 | 0.86 | 0.9241 | 0.9072 | 0.9709 |
| 026_8027_00293330 | Brain | 8027 | D-247MG | 456 | 6.453915 | 0.928 | 0.905 | 0.908 | 0.8398 | 0.8592 | 0.911 | 0.9463 | 0.9529 | 0.9044 |
| 026_346_00283390 | Brain | 346 | A172 | 456 | 6.640511 | 0.941 | 0.953 | 0.995 | 1.0208 | 0.9242 | 1.026 | 1.0141 | 1.0236 | 1.0356 |
| 026_8089_00291170 | Brain | 8089 | KNS-81-FD | 456 | 7.067458 | 0.854 | 0.823 | 0.854 | 0.8437 | 0.8524 | 0.889 | 0.8917 | 0.9075 | 0.9111 |
| 026_355_00285180 | Brain | 355 | U-118MG | 456 | 7.408088 | 0.965 | 1.005 | 0.945 | 0.9352 | 0.9307 | 0.932 | 0.9407 | 0.945 | 0.9851 |
| 026_347_00282740 | Brain | 347 | T98G | 456 | 7.802123 | 0.986 | 1.098 | 0.951 | 1.0602 | 0.9957 | 1.038 | 1.0462 | 0.9753 | 0.982 |
| 026_417_00271110 | Breast | 417 | DU4475 | 456 | −3.0044 | 0.119 | 0.118 | 0.124 | 0.1202 | 0.1235 | 0.121 | 0.164 | 0.4789 | 0.6922 |
| 26_465_00271670 | Breast | 465 | MRK-nu-1 | 456 | 1.744742 | 0.115 | 0.398 | 0.572 | 0.6893 | 0.7807 | 0.868 | 0.921 | 0.9463 | 1.1175 |
| 026_438_00273540 | Breast | 438 | HCC1599 | 456 | 1.968855 | 0.352 | 0.459 | 0.617 | 0.7517 | 0.8021 | 0.899 | 0.9102 | 0.9757 | 1.0216 |
| 026_435_00271290 | Breast | 435 | HCC1187 | 456 | 2.132259 | 0.375 | 0.482 | 0.801 | 0.7466 | 0.7917 | 0.794 | 0.9164 | 0.9352 | 0.9707 |
| 026_403_00271400 | Breast | 403 | MCF7 | 456 | 2.857648 | 0.493 | 0.634 | 0.765 | 0.8761 | 0.9706 | 0.973 | 1.0249 | 1.0247 | 1.0456 |
| 026_401_00273450 | Breast | 401 | MDA-MB-468 | 456 | 3.05753 | 0.415 | 0.718 | 0.931 | 0.9526 | 0.909 | 0.954 | 0.9512 | 1.0282 | 0.9601 |
| 26_451_00271640 | Breast | 451 | CAL-85-1 | 456 | 3.06898 | 0.489 | 0.695 | 0.808 | 0.7646 | 0.8147 | 0.885 | 0.8459 | 1.0885 | 0.9906 |
| 026_404_00273430 | Breast | 404 | MDA-MB-231 | 456 | 3.086092 | 0.483 | 0.731 | 0.783 | 0.824 | 0.8358 | 0.874 | 0.9446 | 0.9609 | 1.0599 |
| 026_418_00271550 | Breast | 418 | Hs 578T | 456 | 3.125956 | 0.071 | 0.614 | 0.832 | 0.7916 | 0.8611 | 0.836 | 0.9569 | 0.9556 | 1.0186 |
| 026_402_00272120 | Breast | 402 | CAMA-1 | 456 | 3.166443 | 0.55 | 0.636 | 0.773 | 0.8152 | 0.9017 | 0.886 | 0.906 | 0.9417 | 0.9695 |
| 026_426_00274200 | Breast | 426 | HCC1569 | 456 | 3.228782 | 0.571 | 0.713 | 0.945 | 0.9056 | 0.9414 | 0.938 | 0.9814 | 1.028 | 1.0046 |
| 026_431_00271130 | Breast | 431 | HCC1806 | 456 | 3.337319 | 0.566 | 0.672 | 0.68 | 0.6852 | 0.6987 | 0.765 | 0.8378 | 1.0081 | 1.1468 |
| 026_414_00271900 | Breast | 414 | AU565 | 456 | 3.409308 | 0.533 | 0.816 | 0.949 | 0.9831 | 0.9841 | 0.924 | 0.9729 | 0.9876 | 1.1475 |
| 026_452_00272130 | Breast | 452 | COLO-824 | 456 | 3.645131 | 0.338 | 0.743 | 0.843 | 0.9519 | 0.9643 | 0.904 | 0.9021 | 1.0288 | 0.9665 |
| 026_416_00271360 | Breast | 416 | BT-549 | 456 | 3.723874 | 0.659 | 0.818 | 0.911 | 0.9492 | 0.9446 | 0.965 | 0.9707 | 0.9785 | 1.0314 |
| 026_8144_00274240 | Breast | 8144 | OCUB-M | 456 | 3.727884 | 0.44 | 0.801 | 0.89 | 0.9127 | 1.0139 | 1.046 | 0.9729 | 0.96 | 1.0167 |
| 026_432_00271960 | Breast | 432 | HCC70 | 456 | 3.73179 | 0.605 | 0.733 | 0.855 | 0.8647 | 0.8214 | 0.887 | 0.9582 | 0.9394 | 1.1987 |
| 026_457_00273420 | Breast | 457 | EVSA-T | 456 | 3.968951 | 0.686 | 0.879 | 0.927 | 0.9385 | 0.9505 | 0.975 | 0.9713 | 0.9606 | 1.0214 |
| 026_466_00274370 | Breast | 466 | YMB-1-E | 456 | 3.997753 | 0.654 | 0.836 | 0.87 | 0.9113 | 0.9082 | 0.908 | 0.9623 | 1.0457 | 0.9738 |
| 026_441_00285120 | Breast | 441 | HCC2157 | 456 | 3.997874 | 0.474 | 1.044 | 0.849 | 0.8244 | 0.6122 | 0.948 | 1.0739 | 0.6695 | 1.0405 |
| 026_443_00271990 | Breast | 443 | MDA-MB-330 | 456 | 4.004084 | 0.601 | 0.92 | 0.94 | 0.9085 | 0.9663 | 0.971 | 0.9817 | 0.9257 | 1.0607 |
| 026_436_00271300 | Breast | 436 | HCC1395 | 456 | 4.036641 | 0.703 | 0.897 | 0.946 | 0.9395 | 1.0136 | 1.033 | 1.0207 | 1.0407 | 1.1296 |
| 026_412_00277190 | Breast | 412 | UACC-893 | 456 | 4.234383 | 0.421 | 0.859 | 0.854 | 0.8448 | 0.9188 | 0.999 | 0.9512 | 1.0422 | 0.9971 |
| 026_450_00271390 | Breast | 450 | CAL-51 | 456 | 4.319545 | 0.678 | 0.725 | 0.783 | 0.8043 | 0.8395 | 0.899 | 0.9313 | 0.9642 | 1.0975 |
| 026_449_00271380 | Breast | 449 | CAL-148 | 456 | 4.389344 | 0.747 | 0.676 | 1.08 | 0.9571 | 0.7093 | 0.888 | 0.981 | 0.8213 | 1.2196 |
| 026_434_00271920 | Breast | 434 | HCC1143 | 456 | 4.464516 | 0.726 | 0.808 | 0.808 | 0.822 | 0.8993 | 0.923 | 0.919 | 0.949 | 1.1427 |
| 026_433_00276270 | Breast | 433 | HCC202 | 456 | 4.571252 | 0.686 | 0.9 | 0.944 | 0.8549 | 0.8115 | 0.943 | 0.972 | 0.9241 | 0.9618 |
| 026_422_00274230 | Breast | 422 | MDA-MB-175- | 456 | 4.594595 | 0.7 | 0.73 | 0.74 | 0.7666 | 0.8545 | 0.848 | 0.9155 | 0.9541 | 0.9487 |
| | | | VII | | | | | | | | | | | |
| 026_461_00272170 | Breast | 461 | MFM-223 | 456 | 4.656681 | 0.787 | 0.875 | 0.908 | 0.9465 | 0.988 | 1.047 | 1.0015 | 1.1431 | 1.0159 |
| 026_427_00271330 | Breast | 427 | MDA-MB-453 | 456 | 4.669025 | 0.795 | 0.819 | 0.921 | 0.9998 | 0.9757 | 0.998 | 1.0349 | 1.0264 | 1.1651 |
| 026_448_00271370 | Breast | 448 | CAL-120 | 456 | 4.779687 | 0.744 | 0.746 | 0.796 | 0.8032 | 0.8728 | 0.85 | 0.9852 | 1.0071 | 1.0287 |
| 026_411_00271420 | Breast | 411 | UACC-812 | 456 | 5.072094 | 0.82 | 0.894 | 0.873 | 0.9564 | 0.9958 | 0.944 | 0.9931 | 1.0326 | 0.9659 |
| 026_442_00273480 | Breast | 442 | HCC2218 | 456 | 5.225292 | 0.614 | 0.923 | 0.963 | 1.0597 | 1.042 | 1.043 | 1.0714 | 1.0498 | 1.0318 |
| 026_398_00272150 | Breast | 398 | HCC1428 | 456 | 5.256241 | 0.85 | 0.899 | 1.013 | 0.8915 | 0.9851 | 0.983 | 0.9959 | 0.9937 | 1.0331 |
| 026_464_00308490 | Breast | 464 | T47D | 456 | 5.26439 | 0.825 | 0.853 | 0.869 | 0.8588 | 0.823 | 0.978 | 0.9397 | 0.9562 | 0.968 |
| 026_400_00273440 | Breast | 400 | MDA-MB-436 | 456 | 5.286367 | 0.725 | 0.844 | 0.848 | 0.8326 | 0.8721 | 0.851 | 0.8881 | 0.9805 | 0.933 |
| 026_437_00280161 | Breast | 437 | HCC1500 | 456 | 5.288806 | 0.796 | 0.982 | 0.913 | 0.8847 | 0.9397 | 1.088 | 0.8523 | 0.9109 | 0.9883 |
| 026_440_00271950 | Breast | 440 | HCC1954 | 456 | 5.303093 | 0.807 | 0.813 | 0.885 | 0.8155 | 0.8463 | 0.861 | 1.0199 | 1.0209 | 0.9481 |
| 026_413_00271930 | Breast | 413 | HCC1419 | 456 | 5.337084 | 0.839 | 0.926 | 0.932 | 0.9634 | 0.9633 | 0.941 | 0.9595 | 0.9516 | 1.1518 |
| 026_410_00272180 | Breast | 410 | ZR-75-30 | 456 | 5.373081 | 0.83 | 0.857 | 0.88 | 0.8529 | 0.9183 | 0.913 | 0.911 | 1.03 | 0.9685 |
| 026_439_00271940 | Breast | 439 | HCC1937 | 456 | 5.44243 | 0.841 | 0.952 | 0.975 | 1.0253 | 0.9632 | 0.953 | 1.0302 | 1.0262 | 1.0774 |
| 026_408_00271350 | Breast | 408 | BT-20 | 456 | 5.735872 | 0.871 | 0.955 | 0.93 | 0.9913 | 0.9764 | 0.972 | 1.0674 | 0.9818 | 1.1818 |
| 026_399_00272160 | Breast | 399 | MDA-MB-415 | 456 | 5.879088 | 0.884 | 0.875 | 0.885 | 0.8851 | 0.9175 | 0.914 | 0.9179 | 0.9049 | 0.9735 |
| 026_458_00274220 | Breast | 458 | HDQ-P1 | 456 | 6.259493 | 0.84 | 1.072 | 1.136 | 1.0763 | 1.0797 | 1.042 | 1.0437 | 1.0231 | 1.0073 |
| 026_397_00274350 | Breast | 397 | HCC38 | 456 | 6.744219 | 0.874 | 1.053 | 1.023 | 1.0222 | 0.9856 | 0.93 | 0.9274 | 0.9402 | 0.9941 |
| 026_405_00274360 | Breast | 405 | MDA-MB-361 | 456 | 6.792889 | 0.935 | 0.959 | 0.987 | 0.9819 | 0.9119 | 0.921 | 0.9597 | 0.998 | 0.9922 |
| 026_425_00280231 | Breast | 425 | MDA-MB-157 | 456 | 7.000504 | 0.906 | 1.069 | 1.05 | 0.9597 | 0.9229 | 0.966 | 0.9422 | 0.9712 | 0.9545 |
| 026_454_00272140 | Breast | 454 | EFM-192A | 456 | 7.097342 | 0.942 | 1.032 | 1.041 | 1.0545 | 0.9812 | 0.979 | 1.0186 | 1.0191 | 0.9742 |
| 026_420_00271541 | Breast | 420 | BT-474 | 456 | 7.458914 | 0.191 | 1.089 | 1.053 | 1.0824 | 1.0008 | 0.955 | 0.9764 | 0.9678 | 1.0149 |
| 026_453_00273410 | Breast | 453 | EFM-19 | 456 | 8.207256 | 1.096 | 1.286 | 1.208 | 1.1112 | 1.0544 | 1.042 | 1.0064 | 1.0474 | 1.0572 |
| 026_415_00316440 | Breast | 415 | BT-483 | 456 | 8.21654 | 1.148 | 1.275 | 1.181 | 1.1462 | 1.089 | 1.147 | 1.0671 | 1.0911 | 1.1123 |
| 026_8176_00316650 | Cervix | 8176 | TC-YIK | 456 | 0.842618 | 0.29 | 0.416 | 0.488 | 0.5211 | 0.5993 | 0.669 | 0.7672 | 0.7877 | 1.0572 |
| 026_479_00264920 | Cervix | 479 | HT-3 | 456 | 1.420025 | 0.257 | 0.608 | 0.574 | 0.5844 | 0.5901 | 0.637 | 0.8649 | 0.8189 | 0.8617 |
| 026_478_00271910 | Cervix | 478 | C-33 A | 456 | 2.72591 | 0.472 | 0.615 | 0.658 | 0.6776 | 0.6753 | 0.734 | 0.7964 | 0.8383 | 0.9143 |
| 026_478_00269410 | Cervix | 478 | C-33 A | 456 | 2.976483 | 0.445 | 0.701 | 0.714 | 0.731 | 0.8172 | 0.771 | 0.9038 | 0.852 | 0.9634 |
| 026_493_00268830 | Cervix | 493 | ME-180 | 456 | 3.07379 | 0.509 | 0.667 | 0.695 | 0.7355 | 0.6828 | 0.732 | 0.7953 | 0.9097 | 1.0456 |
| 026_476_00269050 | Cervix | 476 | C-4 I | 456 | 3.232632 | 0.508 | 0.792 | 0.857 | 0.8645 | 0.8671 | 0.933 | 1.0078 | 1.0484 | 1.1636 |
| 026_8145_00271140 | Cervix | 8145 | OMC-1 | 456 | 3.295968 | 0.531 | 0.786 | 0.84 | 0.8546 | 0.9307 | 0.911 | 1.0261 | 1.0441 | 1.0392 |
| 026_484_00263710 | Cervix | 484 | Ca Ski | 456 | 3.639931 | 0.327 | 0.715 | 0.879 | 0.8811 | 0.8284 | 0.817 | 0.9599 | 1.0361 | 1.165 |
| 026_469_00264610 | Cervix | 469 | HeLa | 456 | 3.981495 | 0.311 | 0.783 | 0.933 | 0.9784 | 0.9807 | 0.982 | 0.9321 | 0.9429 | 0.9946 |
| 026_493_00262480 | Cervix | 493 | ME-180 | 456 | 4.007923 | 0.639 | 0.799 | 0.885 | 0.9474 | 0.9337 | 0.873 | 0.9457 | 0.8694 | 0.8949 |
| 026_474_0029100 | Cervix | 474 | SiHa | 456 | 4.596545 | 0.742 | 0.846 | 0.832 | 0.8807 | 0.9276 | 0.898 | 1.0004 | 0.9967 | 1.1646 |
| 026_482_00262520 | Cervix | 482 | SISO | 456 | 5.313141 | 0.851 | 0.946 | 0.96 | 0.9752 | 0.9888 | 0.276 | 0.975 | 0.947 | 0.9669 |
| 026_482_00274250 | Cervix | 482 | SISO | 456 | 5.375837 | 0.842 | 0.881 | 0.887 | 0.9541 | 0.9605 | 0.96 | 0.9679 | 1.044 | 1.0481 |
| 026_482_00269740 | Cervix | 482 | SISO | 456 | 5.709731 | 0.838 | 0.89 | 0.925 | 0.9202 | 0.9445 | 0.931 | 0.9609 | 0.9695 | 0.9938 |
| 026_468_00264600 | Cervix | 468 | DoTc2 | 456 | 5.749229 | 0.363 | 0.92 | 0.961 | 0.9796 | 0.9907 | 0.984 | 1.01 | 0.9525 | 0.9858 |
| | | | 4510 | | | | | | | | | | | |
| 026_473_00264650 | Cervix | 473 | SW756 | 456 | 5.953892 | 0.162 | 0.868 | 0.813 | 0.7938 | 0.8104 | 0.779 | 0.9163 | 0.8951 | 0.9568 |
| 026_491_00264830 | Cervix | 491 | SKG-IIIa | 456 | 6.261878 | 0.721 | 1.002 | 1.008 | 1.007 | 1.0125 | 0.97 | 1.0049 | 0.9721 | 0.9931 |
| 026_476_00264900 | Cervix | 476 | C-4 I | 456 | 6.792994 | 0.225 | 0.991 | 0.994 | 0.9093 | 0.8797 | 0.89 | 0.982 | 0.9123 | 0.9144 |
| 026_474_00264930 | Cervix | 474 | SiHa | 456 | 7.505839 | 0.532 | 0.824 | 0.865 | 0.9092 | 0.9029 | 0.879 | 0.8904 | 0.8746 | 0.8785 |
| 026_472_00264630 | Cervix | 472 | MS751 | 456 | 7.679336 | 1.023 | 0.945 | 0.949 | 0.9806 | 0.9744 | 0.979 | 0.9915 | 0.9775 | 0.9824 |
| 026_8180_00276230 | Esophagus | 8180 | TE-15 | 456 | 0.823778 | 0.313 | 0.365 | 0.448 | 0.4963 | 0.618 | 0.64 | 0.8902 | 0.6859 | 1.0361 |
| 026_502_00276550 | Esophagus | 502 | KYSE-450 | 456 | 1.219587 | 0.422 | 0.41 | 0.448 | 0.5484 | 0.5334 | 0.703 | 0.7332 | 0.8892 | 0.853 |
| 026_497_00274050 | Esophagus | 497 | KYSE-150 | 456 | 1.304746 | 0.328 | 0.402 | 0.467 | 0.567 | 0.6585 | 0.801 | 0.8957 | 0.9976 | 0.9719 |
| 026_8252_00276570 | Esophagus | 8252 | OACp4C | 456 | 1.678468 | 0.101 | 0.3 | 0.567 | 0.6503 | 0.7826 | 0.861 | 0.8531 | 0.9243 | 0.9446 |
| 026_496_00276530 | Esophagus | 496 | KYSE-140 | 456 | 2.209734 | 0.522 | 0.516 | 0.476 | 0.5824 | 0.6741 | 0.815 | 0.8515 | 0.806 | 0.8896 |
| 026_8233_00278570 | Esophagus | 8233 | ESO26 | 456 | 2.595405 | 0.493 | 0.568 | 0.679 | 0.7293 | 0.7791 | 0.852 | 0.8801 | 1.0449 | 0.8251 |
| 026_8184_00282680 | Esophagus | 8184 | TE-6 | 456 | 2.946928 | 1.152 | 0.677 | 0.704 | 0.6755 | 0.7901 | 0.89 | 1.1989 | 1.0724 | 0.9825 |
| 026_8277_00276670 | Esophagus | 8277 | TE-4 | 456 | 3.072573 | 0.518 | 0.605 | 0.768 | 0.8018 | 0.8549 | 0.883 | 0.9093 | 0.8952 | 0.9579 |
| 026_506_00277170 | Esophagus | 506 | OE19 | 456 | 3.143883 | 0.544 | 0.752 | 0.743 | 0.8799 | 1.0437 | 0.937 | 1.0332 | 1.0004 | 1.0701 |
| 026_8179_00276220 | Esophagus | 8179 | TE-12 | 456 | 3.163617 | 0.183 | 0.742 | 0.633 | 0.6476 | 0.7356 | 0.839 | 0.7964 | 0.8189 | 0.8318 |
| 026_8185_00276250 | Esophagus | 8185 | TE-8 | 456 | 3.50176 | 0.201 | 0.606 | 0.658 | 0.7374 | 0.6587 | 0.744 | 0.775 | 0.681 | 0.9258 |
| 026_8184_00293680 | Esophagus | 8184 | TE-6 | 456 | 3.545508 | 0.552 | 0.767 | 0.935 | 0.8745 | 0.8626 | 0.742 | 1.0239 | 0.863 | 0.9232 |
| 026_8178_00280260 | Esophagus | 8178 | TE-10 | 456 | 3.804003 | 0.664 | 0.735 | 0.78 | 0.9124 | 0.8781 | 1.01 | 1.0331 | 0.9777 | 1.0127 |
| 026_499_00276630 | Esophagus | 499 | KYSE-270 | 456 | 4.276810 | 0.678 | 0.727 | 0.767 | 0.7928 | 0.8774 | 0.984 | 0.9911 | 1.0386 | 1.0007 |
| 026_509_00276620 | Esophagus | 509 | KYSE-220 | 456 | 4.075564 | 0.637 | 0.656 | 0.682 | 0.7368 | 0.7969 | 0.844 | 0.873 | 0.9181 | 0.9229 |
| 026_8235_00276520 | Esophagus | 8235 | FLO-1 | 456 | 4.100409 | 0.604 | 0.732 | 0.778 | 0.7882 | 0.8071 | 0.864 | 0.8729 | 0.8842 | 0.9483 |
| 026_8251_00276640 | Esophagus | 8251 | OACM5-1 | 456 | 4.181038 | 0.673 | 0.784 | 0.878 | 0.8883 | 0.9366 | 0.926 | 0.9214 | 0.9309 | 0.9714 |
| 026_8186_00282690 | Esophagus | 8186 | TE-9 | 456 | 4.291684 | 0.689 | 0.795 | 0.742 | 0.8146 | 0.856 | 0.846 | 1.0123 | 0.8588 | 1.0654 |
| 026_495_00274190 | Esophagus | 495 | COLO-680N | 456 | 4.332573 | 0.742 | 0.818 | 0.865 | 0.9122 | 0.97 | 0.96 | 1.1262 | 0.9684 | 1.0444 |
| 026 510 00273560 | Esophagus | 510 | KYSE-50 | 456 | 4.431288 | 0.658 | 0.743 | 0.781 | 0.8044 | 0.8395 | 0.815 | 0.8665 | 0.9009 | 1.0802 |
| 026_8186_00292740 | Esophagus | 8186 | TE-9 | 456 | 4.438695 | 0.739 | 0.769 | 0.893 | 0.8667 | 0.8573 | 0.944 | 1.0623 | 1.0025 | 1.1458 |
| 026_503_00274070 | Esophagus | 503 | KYSE-510 | 456 | 4.484808 | 0.731 | 0.796 | 0.816 | 0.8942 | 0.9487 | 0.912 | 0.9785 | 1.0104 | 1.0221 |
| 026_504_00276560 | Esophagus | 504 | KYSE-520 | 456 | 4.773857 | 0.716 | 0.731 | 0.748 | 0.7768 | 0.8124 | 0.867 | 0.9459 | 0.995 | 0.9576 |
| 026 8208_00276600 | Esophagus | 8208 | HCE-4 | 456 | 4.88732 | 0.748 | 0.785 | 0.808 | 0.8645 | 0.9033 | 0.954 | 0.9197 | 0.9231 | 0.9878 |
| 026_512_00274080 | Esophagus | 512 | T.T | 456 | 4.943912 | 0.781 | 0.839 | 0.89 | 0.9002 | 0.9769 | 0.929 | 0.9845 | 0.9744 | 1.0152 |
| 026_8268_00276650 | Esophagus | 8268 | SK-GT-4 | 456 | 5.079273 | 0.835 | 0.788 | 0.758 | 0.735 | 0.7503 | 0.833 | 0.9063 | 0.9163 | 0.972 |
| 026_505_00274210 | Esophagus | 505 | KYSE-70 | 456 | 5.137973 | 0.794 | 0.857 | 0.892 | 0.8943 | 0.9411 | 0.957 | 0.9493 | 0.9907 | 1.0431 |
| 026_508_00278520 | Esophagus | 508 | OE33 | 456 | 5.424479 | 0.701 | 0.81 | 0.783 | 0.7873 | 0.7763 | 0.833 | 0.8596 | 0.903 | 0.9287 |
| 026_498_00276540 | Esophagus | 498 | KYSE-180 | 456 | 5.533652 | 0.84 | 0.853 | 0.898 | 0.9109 | 0.9353 | 0.924 | 0.9336 | 0.9199 | 0.942 |
| 026_8202_00276660 | Esophagus | 8202 | TE-11 | 456 | 5.740526 | 0.822 | 0.845 | 0.848 | 0.8684 | 0.8821 | 0.913 | 0.9297 | 0.9627 | 1.0605 |
| 026_8039_00276580 | Esophagus | 8039 | EC-GI-10 | 456 | 5.844841 | 0.878 | 0.905 | 0.892 | 0.951 | 0.9277 | 0.926 | 0.9555 | 1.04 | 1.0068 |
| 026_501_00274060 | Esophagus | 501 | KYSE-410 | 456 | 6.135532 | 0.762 | 0.836 | 0.846 | 0.8398 | 0.8691 | 0.887 | 0.9278 | 0.9729 | 0.9854 |
| 026_8246_00276610 | Esophagus | 8246 | KYAE-1 | 456 | 6.1525 | 0.887 | 0.93 | 0.92 | 0.9326 | 0.9185 | 0.978 | 0.97 | 0.9663 | 0.9315 |
| 026_507_00278510 | Esophagus | 507 | OE21 | 456 | 6.23551 | 0.922 | 0.838 | 0.875 | 0.7771 | 0.8079 | 0.985 | 0.9753 | 0.9667 | 0.9919 |
| 026_8183_00276240 | Esophagus | 8183 | TE-5 | 456 | 6.777236 | 0.111 | 1.028 | 0.936 | 1.0341 | 1.0898 | 0.876 | 0.8843 | 0.9699 | 0.8561 |
| 026_8177_00282670 | Esophagus | 8177 | TE-1 | 456 | 7.848711 | 1.022 | 1.1 | 0.912 | 1.0973 | 1.0839 | 1.065 | 1.0874 | 1.0775 | 1.0749 |
| 026_545_00260020 | Head & | 545 | DOK | 456 | −0.22061 | 0.308 | 0.451 | 0.33 | 0.4014 | 0.4436 | 0.534 | 0.6634 | 0.7589 | 0.8693 |
| | Neck | | | | | | | | | | | | | |
| 026_1217_00255750 | Head & | 1217 | H3118 | 456 | 0.314816 | 0.254 | 0.413 | 0.453 | 0.4692 | 0.5199 | 0.565 | 0.691 | 0.6788 | 0.7738 |
| | Neck | | | | | | | | | | | | | |
| 026_526_00308740 | Head & | 526 | PCI-4B | 456 | 1.887412 | 0.418 | 0.409 | 0.489 | 0.7595 | 0.7429 | 0.873 | 0.9067 | 0.918 | 1.0403 |
| | Neck | | | | | | | | | | | | | |
| 026_530_00260620 | Head & | 530 | PCI-30 | 456 | 1.98819 | 0.142 | 0.356 | 0.628 | 0.7119 | 0.7851 | 0.825 | 0.9139 | 0.8747 | 0.8098 |
| | Neck | | | | | | | | | | | | | |
| 026_552_00252890 | Head & | 552 | SAT | 456 | 2.534279 | 0.382 | 0.613 | 0.727 | 0.8554 | 0.7778 | 0.977 | 0.8474 | 0.7957 | 0.9605 |
| | Neck | | | | | | | | | | | | | |
| 026_550_00258980 | Head & | 550 | SCC-4 | 456 | 2.544916 | 0.464 | 0.564 | 0.904 | 0.7519 | 0.7827 | 0.709 | 0.7715 | 0.8596 | 0.9574 |
| | Neck | | | | | | | | | | | | | |
| 026_1224_00256200 | Head & | 1224 | SCC-9 | 456 | 2.68588 | 0.466 | 0.633 | 0.641 | 0.6635 | 0.6231 | 0.758 | 0.8287 | 0.8854 | 0.9263 |
| | Neck | | | | | | | | | | | | | |
| 026_1223_00259190 | Head & | 1223 | SCC-25 | 456 | 2.934793 | 0.193 | 0.664 | 0.718 | 0.8957 | 0.937 | 0.932 | 1.0111 | 0.988 | 1.0055 |
| | Neck | | | | | | | | | | | | | |
| 026_548_00261030 | Head & | 548 | RPMI 2650 | 456 | 3.095229 | 0.489 | 0.696 | 0.761 | 0.8664 | 0.8685 | 0.911 | 0.9177 | 0.9218 | 0.9624 |
| | Neck | | | | | | | | | | | | | |
| 026_517_00308680 | Head & | 517 | JHU-011 | 456 | 3.112112 | 0.536 | 0.616 | 0.696 | 0.7845 | 0.8433 | 0.813 | 0.9157 | 0.9495 | 1.0827 |
| | Neck | | | | | | | | | | | | | |
| 026_8011_00257080 | Head & | 8011 | BB30-HNC | 456 | 3.217966 | 0.541 | 0.677 | 0.734 | 0.7445 | 0.7909 | 0.917 | 0.9622 | 0.9832 | 1.0203 |
| | Neck | | | | | | | | | | | | | |
| 026_553_00259140 | Head & | 553 | OSC-20 | 456 | 3.378306 | 0.587 | 0.751 | 0.792 | 0.8761 | 0.9722 | 0.983 | 1.0097 | 0.974 | 0.999 |
| | Neck | | | | | | | | | | | | | |
| 026_556_00257220 | Head & | 556 | SKN-3 | 456 | 3.392608 | 0.207 | 0.626 | 0.805 | 0.7174 | 0.6353 | 0.718 | 0.757 | 0.8593 | 0.8383 |
| | Neck | | | | | | | | | | | | | |
| 026_536_00256080 | Head & | 536 | BHY | 456 | 3.443799 | 0.593 | 0.821 | 0.877 | 0.8931 | 0.9405 | 1.049 | 1.0289 | 0.9991 | 1.0281 |
| | Neck | | | | | | | | | | | | | |
| 026_561_00257110 | Head & | 561 | Ca9-22 | 456 | 3.710495 | 0.634 | 0.867 | 0.849 | 0.916 | 0.9493 | 0.99 | 1.0155 | 1.0225 | 1.0222 |
| | Neck | | | | | | | | | | | | | |
| 026_532_00308750 | Head & | 532 | PCI-6A | 456 | 3.742916 | 0.628 | 0.684 | 0.811 | 0.8452 | 0.8862 | 0.945 | 0.9298 | 0.9056 | 0.9974 |
| | Neck | | | | | | | | | | | | | |
| 026_8012_00266550 | Head & | 8012 | BB49-HNC | 456 | 3.77782 | 0.46 | 0.775 | 0.9 | 0.8967 | 0.924 | 0.968 | 0.9717 | 1.0116 | 0.9946 |
| | Neck | | | | | | | | | | | | | |
| 026_8100_00256170 | Head & | 8100 | LB771-HNC | 456 | 3.926882 | 0.701 | 0.899 | 0.929 | 0.9987 | 0.9867 | 0.989 | 1.0145 | 0.9621 | 0.9588 |
| | Neck | | | | | | | | | | | | | |
| 026 1222_00253030 | Head & | 1222 | SCC-15 | 456 | 4.189701 | 0.411 | 0.772 | 0.855 | 0.8291 | 0.7747 | 1.02 | 0.8664 | 0.932 | 0.9717 |
| | Neck | | | | | | | | | | | | | |
| 026_533_00260900 | Head & | 533 | PCI-15A | 456 | 4.236361 | 1.325 | 1.15 | 0.832 | 0.8691 | 0.8188 | 0.863 | 0.9549 | 1.0427 | 1.0471 |
| | Neck | | | | | | | | | | | | | |
| 26_547_00314070 | Head & | 547 | KOSC-2 | 456 | 4.563275 | 0.708 | 0.872 | 0.897 | 0.9 | 0.8378 | 0.941 | 1.0131 | 0.9825 | 0.9292 |
| | Neck | | c13-43 | | | | | | | | | | | |
| 026_544_00256140 | Head & | 544 | Detroit 562 | 456 | 4.601961 | 0.746 | 0.964 | 0.951 | 0.9801 | 0.9656 | 0.961 | 0.9765 | 0.9793 | 0.9061 |
| | Neck | | | | | | | | | | | | | |
| 026_543_00256100 | Head & | 543 | BICR 78 | 456 | 4.851894 | 0.728 | 0.818 | 0.868 | 0.8349 | 0.9283 | 0.925 | 0.9696 | 0.966 | 0.9649 |
| | Neck | | | | | | | | | | | | | |
| 026_537_00256120 | Head & | 537 | CAL-33 | 456 | 5.580216 | 0.834 | 0.892 | 0.921 | 0.9097 | 0.9511 | 0.935 | 0.9977 | 1.0062 | 0.9837 |
| | Neck | | | | | | | | | | | | | |
| 026_549_00256220 | Head & | 549 | HO-1-N-1 | 456 | 5.63511 | 0.866 | 0.873 | 0.883 | 0.8726 | 0.8907 | 0.97 | 0.987 | 0.9898 | 1.0714 |
| | Neck | | | | | | | | | | | | | |
| 026_557_00259180 | Head & | 557 | SAS | 456 | 6.035072 | 0.913 | 0.97 | 0.967 | 0.9738 | 0.9866 | 1.043 | 1.0896 | 1.0407 | 1.0277 |
| | Neck | | | | | | | | | | | | | |
| 026_534_00256110 | Head & | 534 | CAL 27 | 456 | 6.045151 | 0.85 | 0.904 | 0.912 | 0.9312 | 0.925 | 0.939 | 0.9758 | 0.9901 | 0.9927 |
| | Neck | | | | | | | | | | | | | |
| 026_542_00269190 | Head & | 542 | BICR 31 | 456 | 6.045414 | 0.903 | 0.908 | 0.952 | 0.9408 | 0.977 | 0.97 | 0.9966 | 1.0002 | 1.0269 |
| | Neck | | | | | | | | | | | | | |
| 026_530_00262500 | Head & | 530 | PCI-30 | 456 | 6.119674 | 0.884 | 0.955 | 1.145 | 0.9682 | 1.009 | 0.988 | 0.9261 | 1.0121 | 0.9861 |
| | Neck | | | | | | | | | | | | | |
| 026_540_00258490 | Head & | 540 | BICR 10 | 456 | 6.128463 | 0.839 | 1.048 | 1.049 | 1.0556 | 1.0464 | 1.041 | 1.0301 | 0.974 | 0.9953 |
| | Neck | | | | | | | | | | | | | |
| 026_541_00256090 | Head & | 541 | BICR 22 | 456 | 6.156446 | 0.828 | 1.076 | 1.058 | 0.9602 | 1.0898 | 1.064 | 1.0874 | 0.9689 | 1.0462 |
| | Neck | | | | | | | | | | | | | |
| 026_8003_00263440 | Head & | 8003 | A253 | 456 | 6.291179 | 0.877 | 0.857 | 0.859 | 0.8594 | 0.9753 | 0.982 | 0.9985 | 0.9367 | 0.969 |
| | Neck | | | | | | | | | | | | | |
| 026_535_00256160 | Head & | 535 | FaDu | 456 | 6.303565 | 0.841 | 1.063 | 1.091 | 1.0795 | 1.0793 | 1.034 | 1.0404 | 1.0191 | 1.0095 |
| | Neck | | | | | | | | | | | | | |
| 026_554_00257210 | Head & | 554 | OSC-19 | 456 | 6.322489 | 0.938 | 0.942 | 0.914 | 0.9247 | 0.9682 | 1.004 | 1.0285 | 1.0504 | 1.0506 |
| | Neck | | | | | | | | | | | | | |
| 026_559_00256250 | Head & | 559 | HSC-3 | 456 | 6.797258 | 0.909 | 1.024 | 1.057 | 1.0632 | 1.0572 | 1.029 | 1.0059 | 0.9701 | 0.9782 |
| | Neck | | | | | | | | | | | | | |
| 026_8071_00256760 | Head & | 8071 | HCE-T | 456 | 6.917353 | 0.576 | 1.146 | 1.274 | 1.3514 | 1.1688 | 1.173 | 1.0336 | 1.1999 | 0.8849 |
| | Neck | | | | | | | | | | | | | |
| 026_521_00257180 | Head & | 521 | JHU-022 | 456 | 7.049785 | 0.896 | 0.846 | 0.901 | 0.8757 | 0.9278 | 0.886 | 0.913 | 0.9436 | 0.9699 |
| | Neck | | | | | | | | | | | | | |
| 026_555_00256270 | Head & | 555 | KON | 456 | 7.114386 | 0.946 | 0.982 | 0.977 | 0.9826 | 0.9957 | 0.96 | 0.9991 | 0.961 | 0.9207 |
| | Neck | | | | | | | | | | | | | |
| 026_538_00258510 | Head & | 538 | HN | 456 | 7.118392 | 0.947 | 1.007 | 0.984 | 1.0014 | 1.0052 | 0.998 | 1.0105 | 0.9648 | 0.9952 |
| | Neck | | | | | | | | | | | | | |
| 026_546_00259150 | Head & | 546 | PE/CA- | 456 | 7.26219 | 0.96 | 0.963 | 0.931 | 0.9179 | 0.9698 | 0.969 | 0.9884 | 1.006 | 0.9974 |
| | Neck | | PJ15 | | | | | | | | | | | |
| 026_560_00256260 | Head & | 560 | HSC-4 | 456 | 7.41962 | 1.005 | 0.994 | 1.003 | 0.9486 | 1.2114 | 0.897 | 0.9756 | 0.9433 | 0.9255 |
| | Neck | | | | | | | | | | | | | |
| 026_551_00259130 | Head & | 551 | HO-1-u-1 | 456 | 7.903602 | 0.999 | 1.037 | 1.039 | 1.0722 | 1.0616 | 1.067 | 1.0655 | 1.0038 | 0.9405 |
| | Neck | | | | | | | | | | | | | |
| 026_558_00256240 | Head & | 558 | HSC-2 | 456 | 8.185707 | 1.148 | 1.077 | 0.985 | 1.1269 | 1.0034 | 1.093 | 0.9608 | 0.9252 | 0.9888 |
| | Neck | | | | | | | | | | | | | |
| 026_531_00258970 | Head & | 531 | PCI-38 | 456 | 8.493869 | 1.046 | 1.067 | 1.063 | 1.0751 | 1.0712 | 0.965 | 1.0236 | 1.0421 | 0.9848 |
| | Neck | | | | | | | | | | | | | |
| 026_570 00293670 | Intestine | 570 | SK-CO-1 | 456 | −0.30187 | 0.268 | 0.258 | 0.267 | 0.2555 | 0.3743 | 0.418 | 0.7306 | 0.9824 | 0.9276 |
| 026_8153_00295901 | Intestine | 8153 | RKO | 456 | 0.044041 | 0.222 | 0.197 | 0.246 | 0.3161 | 0.4612 | 0.693 | 0.7818 | 0.9061 | 1.1131 |
| 18 | Intestine | 586 | COLO205 | 456 | 0.350012 | 0.083 | 0.182 | 0.268 | 0.3978 | 0.5206 | 0.706 | 0.8518 | 0.9472 | 0.9745 |
| 026_582_00295550 | Intestine | 582 | LoVo | 456 | 0.399717 | 0.325 | 0.396 | 0.385 | 0.4168 | 0.4097 | 0.533 | 0.75 | 0.8239 | 0.9276 |
| 026_8108_00298530 | Intestine | 8108 | LS-513 | 456 | 0.423432 | 0.094 | 0.146 | 0.195 | 0.2527 | 0.5673 | 0.876 | 1.1755 | 1.0179 | 1.1926 |
| 026_8274_00258540 | Intestine | 8274 | SNU-61 | 456 | 0.467862 | 0.432 | 0.599 | 0.539 | 0.5369 | 0.5735 | 0.603 | 0.7283 | 0.7462 | 0.9243 |
| 026_8136_00260060 | Intestine | 8136 | NCI-H747 | 456 | 0.578701 | 0.356 | 0.465 | 0.488 | 0.5338 | 0.5471 | 0.613 | 0.7238 | 0.8107 | 0.9473 |
| 026_574_00298390 | Intestine | 574 | CL-11 | 456 | 0.894127 | 0.381 | 0.401 | 0.395 | 0.4593 | 0.6002 | 0.69 | 0.749 | 0.8474 | 0.9789 |
| 026_589_00295371 | Intestine | 589 | HCT 116 | 456 | 0.931657 | 0.336 | 0.381 | 0.435 | 0.4732 | 0.5791 | 0.862 | 1.1049 | 1.0627 | 1.1072 |
| 026_608_00293620 | Intestine | 608 | CCK-81 | 456 | 1.046144 | 0.242 | 0.394 | 0.399 | 0.4912 | 0.6325 | 0.738 | 0.9653 | 0.9069 | 0.9123 |
| 026_610_00293660 | Intestine | 610 | RCM-1 | 456 | 1.475794 | 0.45 | 0.416 | 0.414 | 0.6252 | 0.6775 | 0.804 | 0.981 | 1.0277 | 1.0565 |
| 026_569_00295390 | Intestine | 569 | HT-29 | 456 | 1.526342 | 0.48 | 0.464 | 0.5 | 0.5547 | 0.5893 | 0.74 | 0.9536 | 0.9048 | 0.9241 |
| 026_8271_00314300 | Intestine | 8271 | SNU-175 | 456 | 1.739925 | 0.382 | 0.522 | 0.535 | 0.5651 | 0.6481 | 0.701 | 0.7957 | 0.9458 | 0.9851 |
| 026_8108_00296610 | Intestine | 8108 | LS-513 | 456 | 1.815428 | 0.37 | 0.448 | 0.511 | 0.6156 | 0.8074 | 0.933 | 0.9905 | 0.9579 | 0.9991 |
| 026_592_00295380 | Intestine | 592 | HT115 | 456 | 1.869689 | 0.377 | 0.436 | 0.5 | 0.6258 | 0.8213 | 1.101 | 1.0858 | 1.0169 | 1.1148 |
| 026_606_00293630 | Intestine | 606 | HCC-56 | 456 | 1.929923 | 0.646 | 0.514 | 0.536 | 0.5915 | 0.7207 | 0.867 | 0.8829 | 0.9131 | 0.9417 |
| 026_8107_00296450 | Intestine | 8107 | LS-411N | 456 | 2.297731 | 0.497 | 0.55 | 0.585 | 0.5585 | 0.6884 | 0.755 | 0.8626 | 0.8921 | 0.9253 |
| 026_595_00295420 | Intestine | 595 | LS180 | 456 | 2.32965 | 0.453 | 0.515 | 0.551 | 0.6999 | 0.8666 | 0.91 | 1.0102 | 0.9178 | 1.1155 |
| 026_8169_00295910 | Intestine | 8169 | SNU-C2B | 456 | 2.617884 | 0.503 | 0.566 | 0.598 | 0.7373 | 0.671 | 0.774 | 0.9066 | 0.8796 | 0.9886 |
| 026_564_00292860 | Intestine | 564 | NCI-H630 | 456 | 2.627492 | 0.496 | 0.556 | 0.55 | 0.7713 | 1.0007 | 0.964 | 0.9563 | 0.8783 | 0.9169 |
| 026_603_00292731 | Intestine | 603 | SW837 | 456 | 2.797981 | 0.525 | 0.597 | 0.586 | 0.7811 | 0.7763 | 1.002 | 1.023 | 1.0218 | 1.0696 |
| 026_588_00295360 | Intestine | 588 | GP5d | 456 | 2.925873 | 0.521 | 0.65 | 0.67 | 0.679 | 0.8325 | 0.964 | 1.0479 | 1.1013 | 1.0678 |
| 026_598_00302650 | Intestine | 598 | W 1417 | 456 | 3.077195 | 0.616 | 0.696 | 0.742 | 0.6531 | 1.034 | 1.029 | 1.0798 | 0.8321 | 1.2207 |
| 026_8276_00296000 | Intestine | 8276 | NU-C5 | 456 | 3.153347 | 0.603 | 0.592 | 0.612 | 0.6327 | 0.7283 | 0.812 | 0.8824 | 0.9411 | 0.9768 |
| 026_593_00295530 | Intestine | 593 | HT55 | 456 | 3.164186 | 0.589 | 0.646 | 0.61 | 0.7857 | 0.7974 | 1.03 | 1.1134 | 1.0941 | 1.1175 |
| 026_8106_00264670 | Intestine | 8106 | LS-123 | 456 | 3.191118 | 0.547 | 0.687 | 0.783 | 0.7554 | 0.8823 | 0.879 | 0.9202 | 0.965 | 0.9375 |
| 026_599_00296340 | Intestine | 599 | W 1463 | 456 | 3.237942 | 0.572 | 0.642 | 0.631 | 0.6255 | 0.6275 | 0.757 | 0.9237 | 0.9401 | 0.9681 |
| 026_8086_00296431 | Intestine | 8086 | KM12 | 456 | 3.240654 | 0.564 | 0.657 | 0.65 | 0.6739 | 0.8062 | 0.903 | 0.8847 | 0.9825 | 0.9793 |
| 026_587_00302320 | Intestine | 587 | COLO 741 | 456 | 3.269221 | 0.615 | 0.622 | 0.694 | 0.6624 | 0.7236 | 0.816 | 1.1047 | 1.0995 | 1.0783 |
| 026_8273_00295970 | Intestine | 8273 | NU-407 | 456 | 3.390403 | 0.577 | 0.622 | 0.663 | 0.7325 | 0.7399 | 0.745 | 0.8059 | 0.8644 | 1.0342 |
| 026_8270_00304630 | Intestine | 8270 | SNU-1040 | 456 | 3.643993 | 0.604 | 0.78 | 0.799 | 0.8256 | 0.8028 | 0.92 | 0.9246 | 0.9919 | 1.2066 |
| 026_8168_00295990 | Intestine | 8168 | NU-C1 | 456 | 3.717495 | 0.645 | 0.673 | 0.685 | 0.6875 | 0.8368 | 0.739 | 0.8513 | 1.1115 | 1.0883 |
| 026_8275_00256210 | Intestine | 8275 | NU-81 | 456 | 3.903109 | 0.338 | 0.659 | 0.68 | 0.6942 | 0.7065 | 0.761 | 0.7749 | 0.8653 | 0.9224 |
| 026_583_00295921 | Intestine | 583 | W-948 | 456 | 3.908038 | 0.735 | 0.672 | 0.851 | 0.9918 | 1.0392 | 0.979 | 0.9178 | 0.9736 | 0.9758 |
| 026_8274_00295980 | Intestine | 8274 | NU-61 | 456 | 4.14154 | 0.663 | 0.72 | 0.672 | 0.6639 | 0.7521 | 0.794 | 0.8775 | 0.8761 | 0.9371 |
| 026_8105_00296440 | Intestine | 8105 | S-1034 | 456 | 4.222062 | 0.732 | 0.754 | 0.781 | 0.8085 | 0.8857 | 1.006 | 1.1032 | 1.0973 | 1.0698 |
| 026_580_00295930 | Intestine | 580 | COLO-678 | 456 | 4.449849 | 0.891 | 0.793 | 0.767 | 0.8394 | 0.8741 | 0.917 | 1.0326 | 0.9344 | 1.0884 |
| 026_580_00266560 | Intestine | 580 | COLO-678 | 456 | 4.550688 | 0.724 | 0.686 | 0.785 | 0.8147 | 0.8701 | 0.885 | 0.9546 | 0.9558 | 0.9803 |
| 026_581_00295830 | Intestine | 581 | CT-15 | 456 | 4.608693 | 0.811 | 0.75 | 0.973 | 0.9405 | 1.0972 | 0.98 | 1.098 | 1.0722 | 1.1405 |
| 026_573_00296370 | Intestine | 573 | SW620 | 456 | 4.816766 | 0.775 | 0.787 | 0.797 | 0.8519 | 0.8683 | 0.915 | 0.9626 | 1.0901 | 1.097 |
| 026_8021_00296390 | Intestine | 8021 | COLO-320-HSR | 456 | 4.87923 | 0.817 | 0.861 | 0.828 | 1.0219 | 0.8974 | 1.103 | 1.0927 | 0.9967 | 1.1145 |
| 026_8106_00298521 | Intestine | 8106 | LS-123 | 456 | 4.895207 | 0.706 | 0.749 | 0.852 | 0.7598 | 0.7605 | 0.923 | 0.8934 | 0.9319 | 1.0175 |
| 026_600_00296361 | Intestine | 600 | SW 48 | 456 | 4.951306 | 0.706 | 0.776 | 0.775 | 0.7937 | 0.8445 | 0.857 | 0.9382 | 0.915 | 0.9609 |
| 026_8070_00296411 | Intestine | 8070 | HCC2998 | 456 | 4.984157 | 0.701 | 0.788 | 0.743 | 0.7463 | 0.8151 | 0.891 | 0.9115 | 0.9572 | 0.9432 |
| 026_8135_00295950 | Intestine | 8135 | NCI-H716 | 456 | 4.988916 | 0.813 | 0.864 | 0.948 | 0.9832 | 0.9658 | 0.94 | 0.9372 | 0.9633 | 1.1237 |
| 026_8136_00295961 | Intestine | 8136 | NCI-H747 | 456 | 5.220086 | 0.699 | 0.693 | 0.763 | 0.7315 | 0.7863 | 0.815 | 0.8715 | 0.8842 | 1.1461 |
| 026_574_00263900 | Intestine | 574 | CL-11 | 456 | 5.294016 | 0.826 | 0.812 | 0.761 | 0.7543 | 0.8087 | 0.891 | 0.9784 | 0.9577 | 0.9649 |
| 026_8026_00300671 | Intestine | 8026 | CW-2 | 456 | 5.403672 | 0.827 | 0.955 | 0.974 | 0.9551 | 0.9288 | 0.954 | 0.9824 | 0.9866 | 1.0636 |
| 026_607_00293610 | Intestine | 607 | CaR-1 | 456 | 5.551737 | 0.834 | 0.873 | 0.912 | 0.8795 | 0.9318 | 0.939 | 0.9636 | 0.9483 | 0.9415 |
| 026_8074_00296420 | Intestine | 8074 | HUTU-80 | 456 | 5.701754 | 0.841 | 0.852 | 0.876 | 0.9027 | 0.8952 | 1.007 | 1.0026 | 0.966 | 0.9322 |
| 026_563_00316540 | Intestine | 563 | 2BBe1 | 456 | 5.783056 | 0.853 | 0.949 | 0.926 | 0.9333 | 0.9253 | 0.879 | 0.9164 | 1.0369 | 1.0733 |
| 026_596_00296280 | Intestine | 596 | DST8 | 456 | 6.402689 | 0.885 | 0.885 | 0.874 | 0.8768 | 0.9374 | 0.931 | 0.9758 | 0.9779 | 1.0734 |
| 026_597_00300651 | Intestine | 597 | W 1116 | 456 | 6.447765 | 0.647 | 1.058 | 0.925 | 1.031 | 0.9839 | 0.997 | 1.0631 | 1.0277 | 0.905 |
| 026_601_00296480 | Intestine | 601 | T84 | 456 | 7.233631 | 0.973 | 0.939 | 0.93 | 0.9736 | 0.9545 | 0.891 | 0.964 | 0.9372 | 1.0259 |
| 026_622_00288160 | Kidney | 622 | G-401 | 456 | 1.158385 | 0.353 | 0.358 | 0.433 | 0.5045 | 0.6202 | 0.865 | 0.8778 | 1.0261 | 1.0061 |
| 026_626_00298790 | Kidney | 626 | BFTC-909 | 456 | 1.589633 | 0.292 | 0.387 | 0.526 | 0.5522 | 0.8154 | 0.895 | 0.9347 | 1.005 | 1.054 |
| 026_623_00288201 | Kidney | 623 | SK-NEP-1 | 456 | 1.837955 | 0.412 | 0.444 | 0.507 | 0.7421 | 0.8141 | 0.813 | 0.9099 | 0.9209 | 0.981 |
| 026_8264_00290630 | Kidney | 8264 | CC-JF | 456 | 2.377362 | 0.452 | 0.535 | 0.585 | 0.6574 | 0.9675 | 0.851 | 0.9828 | 1.0591 | 1.0712 |
| 026_619_00290290 | Kidney | 619 | 769-P | 456 | 2.463867 | 0.439 | 0.58 | 0.576 | 0.7655 | 0.8898 | 0.994 | 1.0776 | 1.0508 | 1.1513 |
| 026_627_00291130 | Kidney | 627 | CAL-54 | 456 | 2.931844 | 0.586 | 0.588 | 0.649 | 0.6813 | 0.7826 | 0.858 | 0.9811 | 0.9726 | 1.0003 |
| 026_617_00290310 | Kidney | 617 | ACHN | 456 | 2.983353 | 0.545 | 0.592 | 0.627 | 0.7345 | 0.7447 | 0.733 | 0.7712 | 0.919 | 1.1152 |
| 026_8263_00290620 | Kidney | 8263 | RCC-FG2 | 456 | 3.111338 | 0.517 | 0.689 | 0.691 | 0.7857 | 0.7896 | 0.884 | 0.8746 | 0.9754 | 1.0284 |
| 026_8190_00290280 | Kidney | 8190 | TK10 | 456 | 3.318654 | 0.638 | 0.654 | 0.671 | 0.7606 | 0.892 | 0.932 | 1.0085 | 1.0009 | 0.9983 |
| 026_638_00288220 | Kidney | 638 | VMRC-RCZ | 456 | 3.36569 | 0.14 | 0.709 | 0.838 | 0.9211 | 0.92 | 0.981 | 0.9922 | 0.9588 | 0.989 |
| 026_8261_00308760 | Kidney | 8261 | RCC-AB | 456 | 3.394556 | 0.566 | 0.724 | 0.758 | 0.85 | 0.8258 | 0.948 | 0.9492 | 0.9471 | 0.9351 |
| 026_628_00288210 | Kidney | 628 | SW 13 | 456 | 3.41636 | 0.454 | 0.733 | 0.735 | 0.7572 | 0.8068 | 0.826 | 0.8584 | 0.9361 | 1.0195 |
| 026_8262_00290610 | Kidney | 8262 | RCC-ER | 456 | 3.745967 | 0.583 | 0.783 | 0.794 | 0.8415 | 0.9082 | 0.87 | 0.8861 | 0.9471 | 0.9855 |
| 026_8265_00302360 | Kidney | 8265 | RCC-JW | 456 | 3.749626 | 0.686 | 0.758 | 0.874 | 0.9595 | 1.0107 | 1.003 | 1.0135 | 1.0068 | 1.0345 |
| 026_8261_00311200 | Kidney | 8261 | RCC-AB | 456 | 3.919417 | 0.288 | 0.72 | 0.73 | 0.765 | 0.8515 | 0.878 | 0.9287 | 0.8965 | 0.9174 |
| 026_618_00290300 | Kidney | 618 | 786-O | 456 | 3.967789 | 0.657 | 0.719 | 0.822 | 0.8016 | 0.8829 | 0.898 | 1.0239 | 0.917 | 1.0472 |
| 026_625_00290670 | Kidney | 625 | UO-31 | 456 | 3.998088 | 0.662 | 0.756 | 0.767 | 0.9002 | 0.8304 | 0.876 | 0.9012 | 1.0588 | 1.1507 |
| 026_8096_00290860 | Kidney | 8096 | LB2241-RCC | 456 | 3.99857 | 0.643 | 0.686 | 0.74 | 0.8137 | 0.8533 | 0.902 | 0.9235 | 0.9107 | 0.9322 |
| 026_614_00291210 | Kidney | 614 | SW 156 | 456 | 4.137243 | 0.651 | 0.665 | 0.71 | 0.7683 | 0.8365 | 0.896 | 0.903 | 0.9074 | 0.9013 |
| 026_8249_00295560 | Kidney | 8249 | NCC021 | 456 | 4.201277 | 0.701 | 0.897 | 0.903 | 0.9199 | 0.9735 | 0.942 | 0.9933 | 1.0276 | 1.0113 |
| 026_633_00290250 | Kidney | 633 | KMRC-20 | 456 | 4.24311 | 0.713 | 0.872 | 0.83 | 0.9644 | 0.8737 | 1.135 | 1.027 | 1.0923 | 1.0373 |
| 026_8068_00290231 | Kidney | 8068 | HA7-RCC | 456 | 4.403879 | 0.761 | 0.764 | 0.7 | 0.8322 | 0.7944 | 0.89 | 1.0474 | 1.1224 | 1.0563 |
| 026_8095_00290260 | Kidney | 8095 | LB1047-RCC | 456 | 4.460236 | 0.772 | 0.824 | 0.914 | 1.0356 | 1.0154 | 1.004 | 1.0739 | 1.1192 | 1.1745 |
| 026_626_00258890 | Kidney | 626 | BFTC-909 | 456 | 4.464205 | 0.741 | 0.932 | 1.057 | 1.0395 | 1.0888 | 1.03 | 1.0217 | 1.0625 | 1.0049 |
| 026_8147_00290270 | Kidney | 8147 | OS-RC-2 | 456 | 4.480976 | 0.767 | 0.794 | 0.799 | 0.8302 | 0.9419 | 1.023 | 1.0718 | 1.0847 | 1.1694 |
| 026_8013_00290220 | Kidney | 8013 | BB65-RCC | 456 | 4.50555 | 0.714 | 0.769 | 0.765 | 0.8909 | 0.8509 | 0.865 | 0.9509 | 1.0096 | 0.903 |
| 026_8006_00293300 | Kidney | 8006 | A704 | 456 | 4.630889 | 0.755 | 0.917 | 0.918 | 0.9328 | 0.9788 | 1.112 | 0.981 | 0.9813 | 1.0413 |
| 026_637_00290240 | Kidney | 637 | KMRC-1 | 456 | 4.786417 | 0.748 | 0.889 | 0.97 | 0.9322 | 0.836 | 1.012 | 1.0239 | 0.9503 | 1.088 |
| 026_624_00290320 | Kidney | 624 | Caki-1 | 456 | 4.808296 | 0.821 | 0.803 | 0.908 | 0.9738 | 0.9722 | 1.083 | 1.1676 | 1.1464 | 1.1228 |
| 026_8266_00290640 | Kidney | 8266 | RCC-MF | 456 | 4.811179 | 0.754 | 0.903 | 0.819 | 0.8455 | 0.8764 | 0.999 | 0.9885 | 0.935 | 1.1762 |
| 026_620_00288170 | Kidney | 620 | G-402 | 456 | 4.865533 | 0.694 | 0.721 | 0.686 | 0.7919 | 0.7766 | 0.804 | 0.8404 | 0.885 | 0.9093 |
| 026_8152_00293381 | Kidney | 8152 | RCC10RGB | 456 | 5.110675 | 0.821 | 0.719 | 0.783 | 0.9032 | 0.8829 | 0.858 | 0.8991 | 0.9287 | 1.0343 |
| 026_640_00291220 | Kidney | 640 | VMRC-RCW | 456 | 5.287926 | 0.73 | 0.737 | 0.793 | 0.8088 | 0.8744 | 0.905 | 0.9003 | 0.9127 | 0.9163 |
| 026_8102_00293360 | Kidney | 8102 | LB996-RCC | 456 | 5.484542 | 0.855 | 0.901 | 0.954 | 0.9116 | 0.9754 | 0.98 | 0.958 | 0.9786 | 0.9921 |
| 026_1119_00290901 | Kidney | 1119 | SN-12C | 456 | 5.586587 | 0.818 | 0.749 | 0.747 | 0.783 | 0.8058 | 0.856 | 0.9038 | 0.9508 | 0.9573 |
| 026_626_00290810 | Kidney | 626 | BFTC-909 | 456 | 5.753321 | 0.799 | 0.854 | 0.903 | 0.9293 | 0.9289 | 0.92 | 0.9333 | 0.9322 | 0.9535 |
| 026_8005_00266530 | Kidney | 8005 | A498 | 456 | 5.843211 | 0.594 | 0.917 | 1.009 | 1.0079 | 0.9307 | 0.996 | 1.0002 | 1.0033 | 0.9941 |
| 026_8157_00296471 | Kidney | 8157 | RXF393 | 456 | 6.166218 | 0.853 | 0.878 | 0.91 | 0.8924 | 0.9071 | 0.932 | 0.9081 | 0.9505 | 0.9889 |
| 026_8102_00253000 | Kidney | 8102 | LB996-RCC | 456 | 6.306723 | 0.959 | 0.873 | 0.878 | 0.9741 | 0.9596 | 1.025 | 0.9525 | 0.9849 | 0.9931 |
| 026_8006_00263880 | Kidney | 8006 | A704 | 456 | 6.969306 | 1.351 | 0.929 | 1.157 | 0.9881 | 1.0206 | 0.957 | 1.0009 | 0.9583 | 0.9931 |
| 026_8152_00256190 | Kidney | 8152 | RCC10RGB | 456 | 7.15535 | 1.178 | 0.835 | 1.176 | 0.846 | 0.8683 | 1.123 | 1.1733 | 1.1056 | 1.0876 |
| 026_8005_00296380 | Kidney | 8005 | A498 | 456 | 8.190783 | 1.022 | 1.025 | 1.024 | 1.0158 | 1.03 | 1.032 | 1.0128 | 1.0239 | 1.0104 |
| 026_233_00277420 | Leukemia | 233 | SIG-M5 | 456 | 5.883853 | 0.228 | 0.243 | 0.233 | 0.2401 | 0.2362 | 0.247 | 0.2641 | 0.2732 | 0.4405 |
| 026_217_00277380 | Leukemia | 217 | OCI-AML2 | 456 | −1.42786 | 0.09 | 0.102 | 0.106 | 0.1256 | 0.1044 | 0.132 | 0.3277 | 0.8333 | 0.8618 |
| 026_179_00314500 | Leukemia | 179 | KMOE-2 | 456 | 0.249088 | 0.13 | 0.135 | 0.138 | 0.1591 | 0.3658 | 0.608 | 0.865 | 0.8967 | 1.0615 |
| 026_214_00285590 | Leukemia | 214 | NB-4 | 456 | −0.011745 | 0.18 | 0.323 | 0.315 | 0.3611 | 0.4215 | 0.592 | 0.8144 | 0.8865 | 0.9464 |
| 026_168_00280410 | Leukemia | 168 | JURL-MK1 | 456 | 0.181843 | 0.145 | 0.179 | 0.186 | 0.2526 | 0.4638 | 0.727 | 0.9379 | 0.904 | 0.9907 |
| 026_233_00277420 | Leukemia | 233 | SIG-M5 | 456 | −5.883853 | 0.228 | 0.243 | 0.233 | 0.2401 | 0.2362 | 0.247 | 0.2641 | 0.2732 | 0.4405 |
| 026_217_00277380 | Leukemia | 217 | OCI-AML2 | 456 | −1.42786 | 0.09 | 0.102 | 0.106 | 0.1256 | 0.1044 | 0.132 | 0.3277 | 0.8333 | 0.8618 |
| 026_179_00314500 | Leukemia | 179 | KMOE-2 | 456 | 0.249088 | 0.13 | 0.135 | 0.138 | 0.1591 | 0.3658 | 0.608 | 0.865 | 0.8967 | 1.0615 |
| 026_214_00285590 | Leukemia | 214 | NB-4 | 456 | 0.011745 | 0.18 | 0.323 | 0.315 | 0.3611 | 0.4215 | 0.592 | 0.8144 | 0.8865 | 0.9464 |
| 026_168_00280410 | Leukemia | 168 | JURL-MK1 | 456 | 0.181843 | 0.145 | 0.179 | 0.186 | 0.2526 | 0.4638 | 0.727 | 0.9379 | 0.904 | 0.9907 |
| 026_194_00280680 | Leukemia | 194 | ML-2 | 456 | 0.294246 | 0.418 | 0.395 | 0.379 | 0.4439 | 0.4739 | 0.633 | 0.7874 | 0.8519 | 1.0223 |
| 026_186_00280670 | Leukemia | 186 | LAMA-84 | 456 | 0.295979 | 0.152 | 0.15 | 0.191 | 0.3623 | 0.6147 | 0.774 | 0.777 | 0.8716 | 0.8816 |
| 026_234_00314650 | Leukemia | 234 | SKM-1 | 456 | 0.349518 | 0.37 | 0.347 | 0.369 | 0.4439 | 0.4995 | 0.57 | 0.729 | 0.9566 | 1.2103 |
| 026_221_00280300 | Leukemia | 221 | OCI-M1 | 456 | 0.656393 | 0.183 | 0.235 | 0.344 | 0.5052 | 0.5942 | 0.759 | 0.8267 | 0.8418 | 1.1212 |
| 026_260_00280430 | Leukemia | 260 | KO52 | 456 | 0.674413 | 0.348 | 0.376 | 0.357 | 0.3446 | 0.4195 | 0.753 | 0.7217 | 0.7637 | 1.0996 |
| 026_45_00274530 | Leukemia | 45 | HL-60 | 456 | 0.756274 | 0.344 | 0.389 | 0.403 | 0.4669 | 0.6154 | 0.78 | 0.9211 | 0.9579 | 1.0107 |
| 026_218_00279140 | Leukemia | 218 | OCI-AML3 | 456 | 0.868641 | 0.368 | 0.38 | 0.401 | 0.4571 | 0.5459 | 0.76 | 0.8359 | 0.9311 | 0.9403 |
| 026_219_00280290 | Leukemia | 219 | OCI-AML5 | 456 | 0.879404 | 0.245 | 0.404 | 0.427 | 0.4943 | 0.686 | 0.712 | 0.8112 | 0.9498 | 0.9021 |
| 026_199_00314510 | Leukemia | 199 | MOLT-13 | 456 | 0.892109 | 0.227 | 0.339 | 0.454 | 0.5097 | 0.5774 | 0.766 | 0.8101 | 0.9633 | 1.1416 |
| 026_226_00280310 | Leukemia | 226 | PL-21 | 456 | 0.938066 | 0.406 | 0.425 | 0.463 | 0.4834 | 0.5377 | 0.657 | 0.7338 | 0.7978 | 0.8482 |
| 026_8141_00274380 | Leukemia | 8141 | NOMO-1 | 456 | 1.040856 | 0.375 | 0.445 | 0.435 | 0.4798 | 0.5465 | 0.636 | 0.9597 | 0.9214 | 0.9288 |
| 026_8069_00279180 | Leukemia | 8069 | HAL-01 | 456 | 1.147185 | 0.349 | 0.345 | 0.454 | 0.634 | 0.6764 | 1.103 | 0.7322 | 0.8261 | 1.0097 |
| 026_68_00273640 | Leukemia | 68 | MV-4-11 | 456 | 1.155322 | 0.061 | 0.367 | 0.445 | 0.5488 | 0.6586 | 0.777 | 0.8697 | 0.9167 | 1.045 |
| 026_175_00282940 | Leukemia | 175 | KARPAS-620 | 456 | 1.190853 | 0.562 | 0.517 | 0.444 | 0.6051 | 0.7411 | 0.916 | 0.9551 | 1.1003 | 1.0193 |
| 026_89_00278880 | Leukemia | 89 | MEG-01 | 456 | 1.211836 | 0.223 | 0.376 | 0.433 | 0.6062 | 0.6931 | 0.86 | 0.9075 | 0.9993 | 1.0376 |
| 026_8017_00279150 | Leukemia | 8017 | BV-173 | 456 | 1.464827 | 0.147 | 0.454 | 0.57 | 0.8503 | 0.5534 | 0.638 | 0.7679 | 0.8629 | 1.0089 |
| 026_225_00277400 | Leukemia | 225 | PF-382 | 456 | 1.646942 | 0.317 | 0.403 | 0.525 | 0.6985 | 0.7396 | 0.932 | 0.9611 | 0.9018 | 1.0833 |
| 026_285_00282960 | Leukemia | 285 | KY821A3 | 456 | 1.656661 | 0.422 | 0.426 | 0.445 | 0.6672 | 0.7837 | 0.935 | 1.0015 | 1.0293 | 1.0583 |
| 026_8008_00280620 | Leukemia | 8008 | ALL-PO | 456 | 1.659142 | 0.143 | 0.469 | 0.549 | 0.6225 | 0.6911 | 0.725 | 0.845 | 0.8482 | 1.019 |
| 026_177_00276860 | Leukemia | 177 | KE-37 | 456 | 1.711407 | 0.195 | 0.451 | 0.523 | 0.6745 | 0.9068 | 0.897 | 0.9087 | 0.9169 | 1.0477 |
| 026_126_00280270 | Leukemia | 126 | GDM-1 | 456 | 1.723253 | 0.297 | 0.444 | 0.595 | 0.682 | 0.7944 | 0.786 | 0.9053 | 0.9081 | 0.8666 |
| 026_261_00274440 | Leukemia | 261 | MY-M12 | 456 | 1.811821 | 0.395 | 0.471 | 0.59 | 0.6443 | 0.7461 | 0.832 | 0.8778 | 1.0023 | 1.0569 |
| 026_201_00277390 | Leukemia | 201 | MOLT-16 | 456 | 1.821177 | 0.276 | 0.43 | 0.578 | 0.7394 | 0.7801 | 0.857 | 0.9379 | 1.0322 | 0.9678 |
| 026_148_00277330 | Leukemia | 148 | CMK | 456 | 1.894859 | 0.275 | 0.484 | 0.637 | 0.6101 | 0.744 | 0.771 | 0.9002 | 0.8949 | 0.8668 |
| 026_28_00280320 | Leukemia | 28 | SUP-B15 | 456 | 1.921651 | 0.261 | 0.506 | 0.579 | 0.7214 | 0.7165 | 0.82 | 0.908 | 0.9679 | 0.9913 |
| 026_190_00287920 | Leukemia | 190 | ME-1 | 456 | 1.924633 | 0.611 | 0.596 | 0.587 | 0.5806 | 0.5987 | 0.684 | 0.7906 | 0.8581 | 0.9106 |
| 026_8150_00273700 | Leukemia | 8150 | QIMR-WIL | 456 | 1.928333 | 0.441 | 0.546 | 0.595 | 0.6574 | 0.7011 | 0.771 | 0.852 | 0.962 | 0.9148 |
| 026_8156_00273710 | Leukemia | 8156 | RPMI-8866 | 456 | 1.939976 | 0.339 | 0.474 | 0.598 | 0.694 | 0.7428 | 0.867 | 0.9518 | 0.9936 | 0.9682 |
| 026_161_00277350 | Leukemia | 161 | HC-1 | 456 | 1.948766 | 0.447 | 0.517 | 0.568 | 0.6165 | 0.6342 | 0.768 | 0.7642 | 0.9074 | 1.0155 |
| 026_195_00279120 | Leukemia | 195 | MOLM-13 | 456 | 1.961596 | 0.394 | 0.455 | 0.518 | 0.6868 | 0.817 | 0.872 | 0.9157 | 0.983 | 1.0345 |
| 026_209_00279130 | Leukemia | 209 | NALM-6 | 456 | 2.000316 | 0.14 | 0.471 | 0.592 | 0.6915 | 0.7587 | 0.808 | 0.8658 | 0.9205 | 0.9768 |
| 026_127_00278810 | Leukemia | 127 | CESS | 456 | 2.000616 | 0.342 | 0.505 | 0.625 | 0.6869 | 0.7679 | 0.843 | 0.9355 | 0.9565 | 0.9827 |
| 026_8196_00278790 | Leukemia | 8186 | 697 | 456 | 2.032634 | 0.337 | 0.476 | 0.617 | 0.7249 | 0.8167 | 0.885 | 1.0481 | 1.0695 | 1.0723 |
| 026_223_00276841 | Leukemia | 223 | P12- | 456 | 2.094412 | 0.266 | 0.472 | 0.62 | 1.0139 | 0.9551 | 0.864 | 0.9219 | 1.0283 | 0.907 |
| | | | ICHIKAWA | | | | | | | | | | | |
| 026_157_00279291 | Leukemia | 157 | DND-41 | 456 | 2.109279 | 0.354 | 0.498 | 0.624 | 0.7174 | 0.8189 | 0.89 | 0.9495 | 0.9928 | 1.0144 |
| 026_174_00276850 | Leukemia | 174 | KARPAS-45 | 456 | 2.156973 | 0.368 | 0.541 | 0.63 | 0.778 | 0.8566 | 0.998 | 1.0142 | 0.9561 | 0.9985 |
| 026_223_00274461 | Leukemia | 223 | P12- | 456 | 2.170641 | 0.274 | 0.465 | 0.567 | 0.909 | 0.9914 | 1.041 | 1.0272 | 0.9908 | 1.0038 |
| | | | ICHIKAWA | | | | | | | | | | | |
| 026_231_00277410 | Leukemia | 231 | RPMI-8402 | 456 | 2.171412 | 0.292 | 0.525 | 0.673 | 0.8379 | 0.894 | 0.956 | 1.0525 | 1.0069 | 1.0163 |
| 026_176_00278840 | Leukemia | 176 | KCL-22 | 456 | 2.178418 | 0.342 | 0.551 | 0.66 | 0.7185 | 0.7521 | 0.822 | 0.8942 | 1.034 | 0.9817 |
| 026_198_00279320 | Leukemia | 198 | MOLP-8 | 456 | 2.182175 | 0.422 | 0.613 | 0.604 | 0.6789 | 0.7123 | 0.753 | 0.8469 | 0.8678 | 0.9555 |
| 026_35_00274541 | Leukemia | 35 | MOLT-4 | 456 | 2.185513 | 0.368 | 0.518 | 0.657 | 0.7557 | 0.8266 | 0.893 | 0.9057 | 1.0649 | 1.0505 |
| 26_256_00273800 | Leukemia | 256 | U266B1 | 456 | 2.191112 | 0.423 | 0.53 | 0.596 | 0.6351 | 0.7316 | 0.782 | 0.8701 | 0.9616 | 0.9089 |
| 026_41_00278850 | Leukemia | 41 | KG-1 | 456 | 2.260524 | 0.445 | 0.507 | 0.569 | 0.7321 | 0.8443 | 0.89 | 0.9006 | 0.9237 | 0.9648 |
| 026_38_00278900 | Leukemia | 38 | THP-1 | 456 | 2.296961 | 0.389 | 0.568 | 0.692 | 0.7667 | 0.7899 | 0.895 | 0.9332 | 0.9649 | 0.9902 |
| 026_153_00277340 | Leukemia | 153 | CTV-1 | 456 | 2.301756 | 0.308 | 0.563 | 0.682 | 0.7882 | 0.8949 | 0.972 | 1.0362 | 1.0213 | 1.0568 |
| 26_284_00273770 | Leukemia | 284 | KY821 | 456 | 2.31293 | 0.459 | 0.524 | 0.599 | 0.642 | 0.7677 | 0.831 | 0.9119 | 0.9596 | 0.9938 |
| 026_256_00304780 | Leukemia | 256 | U266B1 | 456 | 2.374209 | 0.453 | 0.543 | 0.608 | 0.6629 | 0.7604 | 0.806 | 0.9117 | 0.9547 | 0.9487 |
| 026_8033_00279160 | Leukemia | 8033 | DEL | 456 | 2.390133 | 0.433 | 0.595 | 0.647 | 0.7617 | 0.7648 | 0.851 | 0.9096 | 0.9116 | 0.9641 |
| 026_227_00314640 | Leukemia | 227 | RCH-ACV | 456 | 2.405281 | 0.39 | 0.571 | 0.742 | 0.8517 | 0.9043 | 0.946 | 0.9917 | 1.0175 | 1.1197 |
| 026_33_00274521 | Leukemia | 33 | CCRF-CEM | 456 | 2.408403 | 0.354 | 0.606 | 0.685 | 0.7594 | 0.8191 | 0.904 | 0.9597 | 0.9629 | 1.0269 |
| 026_141_00276820 | Leukemia | 141 | K-562 | 456 | 2.457405 | 0.51 | 0.504 | 0.624 | 0.8081 | 0.8379 | 1.163 | 0.9404 | 1.0057 | 1.0193 |
| 026_8137_00273680 | Leukemia | 8137 | NKM-1 | 456 | 2.543263 | 0.464 | 0.601 | 0.622 | 0.6486 | 0.7425 | 0.801 | 0.8993 | 0.9038 | 0.9449 |
| 026_36_00274550 | Leukemia | 36 | Reh | 456 | 2.569808 | 0.418 | 0.628 | 0.742 | 0.8367 | 0.88 | 0.896 | 0.9262 | 0.8979 | 0.9849 |
| 026_8042_00285400 | Leukemia | 8042 | EoL-1-cell | 456 | 2.64235 | 0.232 | 0.569 | 0.651 | 0.7409 | 0.7743 | 0.894 | 0.8333 | 0.905 | 0.9094 |
| 026_281_00273690 | Leukemia | 281 | P30/OHK | 456 | 2.804566 | 0.473 | 0.654 | 0.769 | 0.9121 | 0.8865 | 1.022 | 1.0746 | 1.0233 | 1.0407 |
| 026_59_00278820 | Leukemia | 59 | J.RT3-T3.5 | 456 | 2.830428 | 0.457 | 0.659 | 0.795 | 0.8429 | 0.9259 | 1.002 | 1.0587 | 0.9873 | 1.1396 |
| 026_164_00277360 | Leukemia | 164 | HEL | 456 | 2.835925 | 0.426 | 0.745 | 0.939 | 0.9081 | 1.0194 | 0.987 | 1.0611 | 1.0159 | 0.9841 |
| 026_183_00277370 | Leukemia | 183 | L-363 | 456 | 2.864138 | 0.468 | 0.685 | 0.82 | 0.9025 | 0.9548 | 1.046 | 1.0249 | 0.9812 | 1.02 |
| 026_90_00282840 | Leukemia | 90 | KU812 | 456 | 2.874545 | 0.446 | 0.74 | 0.806 | 0.917 | 0.9589 | 0.978 | 0.9646 | 1.0111 | 1.0409 |
| 026_27_00278890 | Leukemia | 27 | RS4;11 | 456 | 2.914096 | 0.47 | 0.684 | 0.809 | 0.8895 | 0.9144 | 0.975 | 0.9547 | 0.9619 | 1.0194 |
| 026_167_00314490 | Leukemia | 167 | JURKAT | 456 | 2.954834 | 0.503 | 0.713 | 0.842 | 0.9399 | 0.9544 | 0.985 | 0.9987 | 1.034 | 1.0702 |
| 026_181_00278860 | Leukemia | 181 | KOPN-8 | 456 | 2.957727 | 0.484 | 0.669 | 0.731 | 0.7843 | 0.8662 | 0.92 | 0.9473 | 0.9339 | 0.9555 |
| 026_8155_00309110 | Leukemia | 277 | RPMI 8226 | 456 | 2.959542 | 0.648 | 0.63 | 0.689 | 0.8376 | 0.888 | 0.99 | 0.9938 | 1.0182 | 1.0157 |
| 026_8041_00280640 | Leukemia | 8041 | EM-2 | 456 | 3.022652 | 0.557 | 0.687 | 0.614 | 0.634 | 0.706 | 0.679 | 0.8613 | 0.8617 | 1.9538 |
| 026_142_00279280 | Leukemia | 142 | ALL-SIL | 456 | 3.085854 | 0.187 | 0.624 | 0.769 | 0.7092 | 0.7975 | 0.937 | 0.9525 | 0.9717 | 0.8276 |
| 026_277_00280420 | Leukemia | 277 | RPMI 8226 | 456 | 3.102844 | 0.659 | 0.636 | 0.566 | 0.6117 | 0.6594 | 0.722 | 0.8693 | 0.9508 | 0.9057 |
| 026_180_00279310 | Leukemia | 180 | KMS-12-BM | 456 | 3.125028 | 0.512 | 0.704 | 0.788 | 0.8503 | 0.8703 | 0.949 | 0.905 | 0.9806 | 0.9931 |
| 026_8066_00279170 | Leukemia | 8066 | GR-ST | 456 | 3.192114 | 0.532 | 0.681 | 0.762 | 0.8318 | 0.8448 | 0.865 | 1.0258 | 0.9609 | 0.9989 |
| 26_283_00273780 | Leukemia | 283 | LC4-1 | 456 | 3.243599 | 0.592 | 0.732 | 0.916 | 0.9522 | 1.0268 | 1.045 | 0.9517 | 1.0295 | 1.0135 |
| 026_114_00273650 | Leukemia | 114 | SUP-T1 | 456 | 3.397019 | 0.637 | 0.807 | 0.908 | 1.0154 | 0.984 | 1.031 | 0.99 | 0.9877 | 1.0134 |
| 026_138_00287901 | Leukemia | 138 | Loucy | 456 | 3.501279 | 0.289 | 0.749 | 0.772 | 0.942 | 0.9225 | 0.948 | 0.9678 | 0.9653 | 1.0654 |
| 026_8014_00282911 | Leukemia | 8014 | BE-13 | 456 | 3.615529 | 0.68 | 0.757 | 0.965 | 1.1246 | 1.0528 | 1.075 | 1.0884 | 1.0694 | 1.1037 |
| 26_274_00273860 | Leukemia | 274 | BALL-1 | 456 | 3.704228 | 0.65 | 0.709 | 0.777 | 0.8768 | 0.9725 | 0.983 | 0.9819 | 0.9839 | 0.9978 |
| 026_222_00280480 | Leukemia | 228 | OPM-2 | 456 | 3.757754 | 0.681 | 0.619 | 0.771 | 0.9443 | 0.8789 | 1.073 | 0.9014 | 0.9191 | 0.9051 |
| 026_8164_00283540 | Leukemia | 8164 | SK-MM-2 | 456 | 3.790344 | 0.669 | 0.788 | 0.861 | 0.8903 | 0.938 | 0.941 | 1.0087 | 0.9277 | 0.9972 |
| 026_166_00278830 | Leukemia | 166 | JJN-3 | 456 | 3.901981 | 0.63636 | 0.791 | 0.85 | 0.8566 | 0.8711 | 0.916 | 0.9455 | 0.9892 | 1.057 |
| 026_230_00282970 | Leukemia | 230 | ROS-50 | 456 | 4.130585 | 0.741 | 0.775 | 0.805 | 0.9413 | 0.9813 | 1.11 | 1.1334 | 1.0559 | 0.999 |
| 026_159_00314480 | Leukemia | 159 | EJM | 456 | 4.251735 | 0.714 | 0.845 | 0.899 | 0.9099 | 1.0285 | 0.923 | 1.0677 | 1.0768 | 1.0543 |
| 026_278_00304730 | Leukemia | 278 | KMS-12-PE | 456 | 4.269602 | 0.765 | 0.814 | 0.84 | 1.0641 | 0.988 | 1.023 | 1.0045 | 1.0284 | 1.0665 |
| 026_8219_00282850 | Leukemia | 8219 | Mo-T | 456 | 4.313664 | 0.73 | 0.825 | 0.804 | 0.9425 | 0.9233 | 1.033 | 0.8454 | 1.1551 | 1.0561 |
| 026_279_00274450 | Leukemia | 279 | P31/FUJ | 456 | 4.386897 | 0.705 | 0.805 | 0.799 | 0.819 | 0.8632 | 1.043 | 1.0364 | 0.9822 | 1.0363 |
| 026_171_00285190 | Leukemia | 171 | KARPAS-231 | 456 | 4.404825 | 0.703 | 0.9 | 0.906 | 0.9339 | 0.9295 | 0.904 | 0.9725 | 0.9496 | 0.9164 |
| 026_244_00273660 | Leukemia | 244 | TALL-1 | 456 | 4.411016 | 0.698 | 0.758 | 0.785 | 0.8345 | 0.8593 | 0.861 | 1.0445 | 1.0026 | 1.0194 |
| 026_158_00291340 | Leukemia | 158 | EHEB | 456 | 4.509743 | 0.665 | 0.745 | 0.765 | 0.7841 | 0.8282 | 0.839 | 0.92 | 0.9348 | 1.0344 |
| 026_134_00278800 | Leukemia | 134 | ARH-77 | 456 | 4.545002 | 0.781 | 0.856 | 0.96 | 0.848 | 1.0568 | 1.029 | 1.0104 | 1.0156 | 1.0086 |
| 026_246_00273670 | Leukemia | 246 | U-698-M | 456 | 4.580953 | 0.754 | 0.891 | 0.936 | 0.9419 | 0.9607 | 0.937 | 1.032 | 0.9352 | 1.0229 |
| 026_8113_00280440 | Leukemia | 8113 | MHH-CALL-2 | 456 | 4.648418 | 0.771 | 0.883 | 0.929 | 0.9704 | 0.9337 | 0.987 | 1.0345 | 1.0223 | 1.0471 |
| 026_159_00311690 | Leukemia | 159 | EJM | 456 | 4.699945 | 0.746 | 0.894 | 0.921 | 0.9275 | 0.9393 | 0.941 | 0.9575 | 0.9338 | 0.9675 |
| 026_159_00282920 | Leukemia | 159 | EJM | 456 | 4.832393 | 0.746 | 0.976 | 1.016 | 0.9859 | 1.0015 | 1.002 | 1.1587 | 1.1041 | 1.0548 |
| 026_8115_00280450 | Leukemia | 8115 | MN-60 | 456 | 4.861437 | 0.826 | 0.804 | 0.822 | 1.0586 | 0.9555 | 0.947 | 1.0471 | 1.128 | 1.0314 |
| 026_204_00280460 | Leukemia | 204 | MONO- | 456 | 4.946039 | 0.736 | 0.802 | 0.771 | 0.8409 | 0.8712 | 0.833 | 0.8634 | 1.0677 | 0.855 |
| | | | MAC-6 | | | | | | | | | | | |
| 026_8117_00280470 | Leukemia | 8117 | MUTZ-1 | 456 | 5.304526 | 0.823 | 0.984 | 1.063 | 1.051 | 1.0142 | 1.007 | 0.9986 | 0.9654 | 0.9525 |
| 026_188_00282930 | Leukemia | 188 | LP-1 | 456 | 5.373997 | 0.831 | 0.887 | 0.972 | 0.8808 | 0.9105 | 0.886 | 0.9533 | 0.9576 | 1.138 |
| 026_8081_00285580 | Leukemia | 8081 | JVM-3 | 456 | 5.581328 | 0.884 | 0.904 | 0.907 | 0.9131 | 1.01 | 1.097 | 1.0593 | 0.9629 | 1.0333 |
| 026_159_00309070 | Leukemia | 159 | EJM | 456 | 5.810283 | 0.882 | 0.986 | 0.998 | 1.0992 | 1.0048 | 1.045 | 1.0126 | 1.024 | 1.0649 |
| 026_8080_00280660 | Leukemia | 8080 | JVM-2 | 456 | 5.982881 | 0.867 | 0.876 | 0.901 | 0.8865 | 0.9235 | 0.909 | 0.9782 | 0.8964 | 0.9789 |
| 026_278_00306920 | Leukemia | 278 | KMS-12-PE | 456 | 6.629507 | 0.971 | 0.92 | 0.95 | 0.9554 | 0.9046 | 0.905 | 0.9657 | 1.0243 | 0.9236 |
| 026_8010_00280630 | Leukemia | 8010 | ATN-1 | 456 | 7.14863 | 1.03 | 0.958 | 0.835 | 0.8896 | 0.8651 | 0.907 | 0.9392 | 0.8582 | 0.9763 |
| 26_262_00273790 | Leukemia | 262 | MLMA | 456 | 7.68552 | 0.485 | 1.033 | 1.077 | 1.0216 | 1.0341 | 0.962 | 1.0378 | 0.9821 | 1.0245 |
| 026_278_00282950 | Leukemia | 278 | KMS-12-PE | 456 | 7.74945 | 0.958 | 1.101 | 1.04 | 1.1177 | 1.0046 | 1.029 | 1.0272 | 1.0311 | 1.0037 |
| 026_649_00264910 | Liver | 649 | Hep3B2.1-7 | 456 | −0.30042 | 0.134 | 0.098 | 0.087 | 0.1121 | 0.2203 | 0.601 | 0.8432 | 0.8926 | 0.9089 |
| 026_658_00262810 | Liver | 658 | JHH-1 | 456 | −0.185127 | 0.054 | 0.329 | 0.515 | 0.3993 | 0.5194 | 0.524 | 0.6608 | 0.6499 | 0.6863 |
| 026_649_00266180 | Liver | 649 | Hep3B2.1-7 | 456 | −0.037335 | 0.073 | 0.105 | 0.093 | 0.0908 | 0.1037 | 0.202 | 0.8123 | 0.9207 | 1.007 |
| 026_667_00273550 | Liver | 667 | HuH-7 | 456 | 1.289162 | 0.279 | 0.565 | 0.539 | 0.5535 | 0.7134 | 0.77 | 0.8626 | 0.9675 | 0.9662 |
| 026_659_00255780 | Liver | 659 | JHH-2 | 456 | 1.758427 | 0.283 | 0.473 | 0.657 | 0.7149 | 0.551 | 0.819 | 0.8858 | 0.8891 | 1.0202 |
| 026_643_00266460 | Liver | 643 | SNU-398 | 456 | 2.578054 | 0.415 | 0.687 | 0.643 | 0.6854 | 0.6879 | 0.717 | 0.7896 | 0.8076 | 0.9193 |
| 026_667_00269210 | Liver | 667 | HuH-7 | 456 | 2.760591 | 0.501 | 0.59 | 0.665 | 0.6852 | 0.6886 | 0.781 | 0.95 | 1.0287 | 1.0142 |
| 026_647_00269110 | Liver | 647 | SNU-387 | 456 | 2.930627 | 0.494 | 0.772 | 0.829 | 0.9402 | 0.9686 | 1.046 | 1.0372 | 1.0223 | 1.2144 |
| 026_661_00252500 | Liver | 661 | JHH-7 | 456 | 3.286573 | 0.539 | 0.889 | 0.794 | 0.8777 | 1.0776 | 0.953 | 0.9918 | 1.037 | 0.9476 |
| 026_643_00263980 | Liver | 643 | SNU-398 | 456 | 3.293137 | 0.487 | 0.768 | 0.854 | 0.8235 | 0.8763 | 0.883 | 0.9616 | 1.068 | 1.0718 |
| 026_642_00308440 | Liver | 642 | C3A | 456 | 3.554832 | 0.587 | 0.68 | 0.706 | 0.7687 | 0.817 | 0.878 | 0.9697 | 0.9574 | 0.9245 |
| 026_648_00258350 | Liver | 648 | SNU-423 | 456 | 3.576003 | 0.139 | 0.699 | 0.902 | 0.9157 | 0.9485 | 0.909 | 0.9218 | 0.9469 | 1.0028 |
| 026_656_00252510 | Liver | 656 | JHH-4 | 456 | 3.61979 | 0.701 | 0.653 | 0.631 | 0.8187 | 0.7968 | 0.977 | 0.9161 | 0.9897 | 0.9288 |
| 026_660_00252490 | Liver | 660 | JHH-6 | 456 | 3.831185 | 0.696 | 0.88 | 1.034 | 0.9294 | 0.9465 | 1.04 | 0.9417 | 1.0528 | 0.952 |
| 026_644_00252750 | Liver | 644 | SNU-449 | 456 | 4.40481 | 0.664 | 0.733 | 0.764 | 0.7954 | 0.8597 | 0.896 | 0.9485 | 0.9457 | 0.9162 |
| 026_644_00306170 | Liver | 644 | SNU-449 | 456 | 6.450644 | 0.96 | 0.961 | 0.745 | 1.0716 | 1.0197 | 1.08 | 0.991 | 0.9007 | 0.9369 |
| 26_646_00314060 | Liver | 646 | SNU-475 | 456 | 6.501654 | 0.181 | 0.97 | 0.971 | 0.9863 | 1.0061 | 1.007 | 1.0271 | 1.0202 | 0.057 |
| 026_654_00255800 | Liver | 654 | SK-HEP-1 | 456 | 6.515068 | 0.927 | 0.956 | 0.961 | 0.9827 | 0.9589 | 0.98 | 0.9783 | 0.9726 | 0.9998 |
| 026_668_00252690 | Liver | 668 | HLE | 456 | 7.25378 | 0.434 | 1.006 | 1.087 | 0.9897 | 1.1103 | 0.964 | 1.006 | 1.106 | 0.9541 |
| 026_662_00252460 | Liver | 662 | huH-1 | 456 | 7.722693 | 0.466 | 1.165 | 1.072 | 1.0401 | 1.0408 | 1.136 | 1.015 | 1.123 | 0.9778 |
| 026_645_00306160 | Liver | 645 | SNU-182 | 456 | 7.852049 | 1.105 | 1.061 | 1.063 | 1.0055 | 1.0268 | 1.053 | 1.0365 | 1.0318 | 1.0842 |
| 026_642_00252670 | Liver | 642 | C3A | 456 | 7.864415 | 0.386 | 1.1 | 1.007 | 1.108 | 0.9463 | 1.051 | 0.9803 | 1.029 | 0.9218 |
| 026_830_00304760 | Lung | 830 | NCI-H2135 | 456 | −0.374655 | 0.298 | 0.262 | 0.246 | 0.3049 | 0.3692 | 0.528 | 0.7269 | 0.8905 | 0.977 |
| 026_698_00300170 | Lung | 698 | NCI-H524 | 456 | 2.279246 | 0.443 | 0.506 | 0.63 | 0.8741 | 0.9302 | 1.005 | 1.0131 | 1.0304 | 1.0567 |
| 026_672_00314460 | Lung | 672 | NCI-H510A | 456 | 2.367223 | 0.323 | 0.585 | 0.763 | 0.783 | 0.8906 | 0.969 | 0.9972 | 1.1809 | 1.0131 |
| 026_761_00300410 | Lung | 761 | COR-L279 | 456 | 3.131703 | 0.559 | 0.745 | 0.868 | 0.9821 | 1.0079 | 1.06 | 1.0144 | 1.0182 | 1.0709 |
| 026_726_00304770 | Lung | 726 | NCI-H2171 | 456 | 3.185965 | 0.524 | 0.729 | 0.842 | 0.8655 | 0.9088 | 0.871 | 1.0252 | 0.9207 | 1.129 |
| 026_740_00302760 | Lung | 740 | NCI-H82 | 456 | 3.210581 | 0.542 | 0.755 | 0.865 | 0.9592 | 1.0522 | 0.888 | 0.9137 | 1.004 | 1.0151 |
| 026_787_00302910 | Lung | 787 | SBC-3 | 456 | 3.410219 | 0.582 | 0.655 | 0.694 | 0.802 | 0.8502 | 0.902 | 0.9485 | 0.9451 | 0.9885 |
| 026_695_00300150 | Lung | 695 | NCI-H211 | 456 | 3.515811 | 0.644 | 0.645 | 0.719 | 0.8327 | 0.8177 | 0.933 | 0.9519 | 0.9812 | 1.0305 |
| 026_721_00302860 | Lung | 721 | NCI-H2029 | 456 | 3.591515 | 0.606 | 0.8 | 0.891 | 0.9011 | 0.8579 | 0.931 | 0.9429 | 1.0032 | 1.0011 |
| 026_776_00303250 | Lung | 776 | MS-1-L | 456 | 3.614256 | 0.642 | 0.767 | 0.855 | 0.8458 | 1.1115 | 0.929 | 1.0414 | 1.0285 | 1.162 |
| 026_8197_00304741 | Lung | 8197 | LU-139 | 456 | 3.667637 | 0.66 | 0.84 | 0.901 | 1.0158 | 0.9097 | 0.912 | 1.0844 | 1.1048 | 1.0641 |
| 026_702_00302900 | Lung | 702 | NCI-H847 | 456 | 3.686524 | 0.818 | 0.707 | 0.668 | 0.6815 | 0.7184 | 0.743 | 0.7999 | 0.9145 | 0.9524 |
| 026_8203_00309050 | Lung | 8203 | IST-SL1 | 456 | 3.697133 | 0.635 | 0.801 | 0.87 | 0.931 | 0.9112 | 1 | 1.0127 | 0.9507 | 1.0704 |
| 026_724_00300140 | Lung | 724 | NCI-H2081 | 456 | 3.857634 | 0.623 | 0.866 | 0.895 | 0.9027 | 0.9349 | 0.948 | 0.969 | 0.9681 | 1.0072 |
| 026_765_00300050 | Lung | 765 | DMS 273 | 456 | 3.890251 | 0.702 | 0.844 | 0.904 | 0.9648 | 1.028 | 0.968 | 1.0795 | 1.0165 | 1.0825 |
| 026_829_00305160 | Lung | 829 | NCI-H2110 | 456 | 3.972695 | 0.67 | 0.694 | 0.776 | 0.8765 | 0.8765 | 0.946 | 1.0573 | 0.91 | 0.9398 |
| 026_742_00303230 | Lung | 742 | DMS 53 | 456 | 3.990104 | 0.711 | 0.802 | 0.867 | 0.9734 | 1.0075 | 1.037 | 1.0307 | 1.0204 | 1.0405 |
| 026_710_00316710 | Lung | 710 | NCI-H1341 | 456 | 4.024081 | 0.591 | 0.852 | 0.7 | 0.7407 | 0.8259 | 0.789 | 0.8069 | 0.8386 | 0.865 |
| 026_738_00302800 | Lung | 738 | NCI-H446 | 456 | 4.043153 | 0.692 | 0.871 | 0.917 | 1.0421 | 0.9341 | 0.915 | 1.0657 | 0.9649 | 1.0382 |
| 026_751_00308570 | Lung | 751 | NCI-H209 | 456 | 4.061511 | 0.657 | 0.848 | 0.89 | 0.9403 | 0.8939 | 0.91 | 0.9316 | 0.9524 | 0.9513 |
| 026_716_00298900 | Lung | 716 | NCI-H1876 | 456 | 4.083964 | 0.744 | 0.866 | 0.936 | 0.9448 | 0.9881 | 0.979 | 0.9782 | 0.9994 | 1.0261 |
| 026_725_00303280 | Lung | 725 | NCI-H2141 | 456 | 4.09696 | 0.711 | 0.842 | 0.928 | 0.9702 | 0.9155 | 0.924 | 1.0044 | 1.0101 | 0.9559 |
| 026_688_00302810 | Lung | 688 | SW 1271 | 456 | 4.124557 | 0.602 | 0.761 | 0.79 | 0.8093 | 0.807 | 0.823 | 0.896 | 0.8782 | 0.9341 |
| 026_720_00302380 | Lung | 720 | NCI-H1994 | 456 | 4.130367 | 0.696 | 0.694 | 0.73 | 0.8114 | 0.9245 | 0.935 | 0.9484 | 0.9393 | 0.9738 |
| 026_811_00311720 | Lung | 811 | NCI-H1435 | 456 | 4.138326 | 0.635 | 0.803 | 0.855 | 0.8503 | 0.8571 | 0.89 | 0.8912 | 0.955 | 0.9956 |
| 026_704_00300250 | Lung | 704 | NCI-H1048 | 456 | 4.178908 | 0.166 | 0.762 | 0.867 | 0.8914 | 0.9318 | 0.923 | 0.9201 | 1.0122 | 0.9635 |
| 026_746_00302780 | Lung | 746 | SHP-77 | 456 | 4.20487 | 0.79 | 0.843 | 0.955 | 1.029 | 0.9896 | 1.021 | 1.0111 | 0.9931 | 1.0025 |
| 026_829_00311740 | Lung | 829 | NCI-H2110 | 456 | 4.22315 | 0.739 | 0.83 | 0.914 | 0.9675 | 0.9597 | 0.983 | 0.9791 | 1.032 | 0.9995 |
| 026_736_00300181 | Lung | 736 | NCI-H69 | 456 | 4.26966 | 0.798 | 0.87 | 0.945 | 1.0535 | 1.0472 | 1.143 | 1.0521 | 1.0846 | 1.1743 |
| 026_724_00303270 | Lung | 724 | NCI-H2081 | 456 | 4.274134 | 0.708 | 0.95 | 1.023 | 0.9666 | 1.0134 | 0.99 | 1.0037 | 1.0388 | 1.0531 |
| 026_714_00300260 | Lung | 714 | NCI-H1694 | 456 | 4.276996 | 0.701 | 0.831 | 0.843 | 0.8708 | 0.9426 | 0.93 | 0.9095 | 1.1071 | 1.0848 |
| 026_715_00298890 | Lung | 715 | NCI-H1836 | 456 | 4.291554 | 0.73 | 0.833 | 0.997 | 0.9315 | 0.9434 | 0.955 | 0.9291 | 0.9423 | 1 |
| 026_757_00302870 | Lung | 757 | CPC-N | 456 | 4.345126 | 0.718 | 0.819 | 0.887 | 0.8883 | 0.9167 | 0.938 | 0.9641 | 1.0154 | 1.0153 |
| 026_8229_00304990 | Lung | 8229 | COR-L303 | 456 | 4.468573 | 0.685 | 0.828 | 0.788 | 0.868 | 0.6881 | 0.855 | 0.9579 | 0.8466 | 0.9772 |
| 026_691_00308560 | Lung | 691 | NCI-H526 | 456 | 4.602758 | 0.748 | 0.932 | 0.944 | 0.9861 | 0.9744 | 0.954 | 1.0035 | 1.0955 | 1.0007 |
| 026_8099_00316740 | Lung | 8099 | LB647-SCLC | 456 | 4.613258 | 0.755 | 0.954 | 0.998 | 0.9372 | 0.957 | 1.041 | 1.0171 | 1.0259 | 0.9506 |
| 026_725_00302750 | Lung | 725 | NCI-H2141 | 456 | 4.618451 | 0.747 | 0.869 | 0.906 | 0.9009 | 0.9283 | 0.901 | 1.003 | 0.9238 | 1.1424 |
| 026_705_00314520 | Lung | 705 | NCI-H1092 | 456 | 4.678169 | 0.77 | 0.861 | 0.911 | 1.02 | 0.9158 | 1.082 | 0.9997 | 0.9139 | 0.961 |
| 026_684_00303260 | Lung | 684 | NCI-H1688 | 456 | 4.865947 | 0.832 | 0.831 | 0.988 | 1.0672 | 0.9906 | 1.099 | 1.0391 | 1.0378 | 1.0698 |
| 026_725_00300160 | Lung | 725 | NCI-H2141 | 456 | 4.894458 | 0.785 | 0.908 | 0.936 | 0.9468 | 0.9309 | 0.94 | 0.9635 | 1.0477 | 0.9963 |
| 026_8281_00300930 | Lung | 8281 | COR-L311 | 456 | 5.024667 | 0.792 | 0.892 | 0.947 | 0.9155 | 0.8604 | 0.871 | 1.0268 | 1.0989 | 1.045 |
| 026_757_00300940 | Lung | 757 | CPC-N | 456 | 5.046784 | 0.796 | 0.904 | 0.984 | 1.0041 | 1.0137 | 0.919 | 0.905 | 0.9063 | 1.0167 |
| 026_701_00309060 | Lung | 701 | NCI-H841 | 456 | 5.157768 | 0.826 | 0.808 | 0.939 | 0.891 | 0.8811 | 0.919 | 0.9514 | 1.0474 | 1.0933 |
| 026_814_00304750 | Lung | 814 | NCI-H1568 | 456 | 5.159278 | 0.784 | 0.726 | 0.739 | 0.745 | 0.7127 | 0.774 | 0.9411 | 0.9 | 0.9817 |
| 026_741_00305010 | Lung | 741 | NCI-H345 | 456 | 5.184481 | 0.801 | 0.818 | 0.835 | 0.9503 | 0.9116 | 0.894 | 0.8849 | 0.9853 | 1.083 |
| 026_705_00311700 | Lung | 705 | NCI-H1092 | 456 | 5.191642 | 0.849 | 0.892 | 0.945 | 1.0426 | 0.9184 | 0.981 | 1.0366 | 1.0158 | 1.0581 |
| 026_786_00300950 | Lung | 786 | SBC-5 | 456 | 5.281883 | 0.841 | 1.002 | 0.991 | 1.0148 | 0.9974 | 0.998 | 1.0298 | 1.0075 | 1.0015 |
| 026_723_00316720 | Lung | 723 | NCI-H2066 | 456 | 5.313778 | 0.793 | 0.815 | 0.866 | 0.86 | 0.9117 | 0.892 | 0.9084 | 0.923 | 0.9757 |
| 026_705_00309080 | Lung | 705 | NCI-H1092 | 456 | 5.329516 | 0.829 | 0.941 | 0.999 | 0.9672 | 0.8963 | 0.935 | 0.9991 | 0.9888 | 1.0506 |
| 026_709_00305000 | Lung | 709 | NCI-H1304 | 456 | 5.330039 | 0.838 | 0.882 | 0.906 | 0.8939 | 0.9384 | 0.978 | 1.0127 | 1.0322 | 1.0575 |
| 026_739_00314630 | Lung | 739 | NCI-H146 | 456 | 5.38813 | 0.858 | 0.893 | 0.978 | 1.0115 | 1.0583 | 0.937 | 0.9685 | 1.0018 | 1.0831 |
| 026_8110_00314450 | Lung | 8110 | LU-165 | 456 | 5.539798 | 0.87 | 0.909 | 0.986 | 0.9964 | 0.9809 | 1.094 | 0.9305 | 0.9513 | 1.1416 |
| 026_811_00305140 | Lung | 811 | NCI-H1435 | 456 | 5.586661 | 0.886 | 0.953 | 0.978 | 0.9914 | 1.0102 | 0.994 | 0.9871 | 0.9783 | 0.9731 |
| 026_711_00311710 | Lung | 711 | NCI-H1417 | 456 | 5.606717 | 0.875 | 0.91 | 0.911 | 0.9612 | 1.0374 | 0.917 | 1.267 | 1.0195 | 0.9447 |
| 026_712_00309090 | Lung | 712 | NCI-H1436 | 456 | 5.636542 | 0.85 | 0.918 | 0.976 | 0.9527 | 0.9331 | 0.932 | 0.9583 | 0.9477 | 1.0343 |
| 026_711_00305130 | Lung | 711 | NCI-H1417 | 456 | 5.804325 | 0.849 | 1.022 | 0.916 | 0.9247 | 0.9771 | 0.939 | 0.9326 | 1.0168 | 1.0143 |
| 026_728_00311760 | Lung | 728 | NCI-H2196 | 456 | 5.845371 | 0.89 | 0.908 | 0.918 | 0.9801 | 0.875 | 1.033 | 0.9519 | 0.931 | 1.0431 |
| 026_831_00311750 | Lung | 831 | NCI-H2172 | 456 | 5.851811 | 0.879 | 0.986 | 1.002 | 0.9709 | 0.9562 | 0.984 | 0.9781 | 1.0144 | 0.974 |
| 026_1216_00300060 | Lung | 1216 | H292 | 456 | 5.909493 | 0.869 | 0.823 | 0.808 | 0.8192 | 0.8429 | 0.864 | 0.9245 | 0.9789 | 1.0777 |
| 026_8109_00314440 | Lung | 8109 | LU-134-A | 456 | 5.930549 | 0.868 | 0.95 | 0.972 | 0.9375 | 0.9332 | 0.959 | 0.9516 | 1.1207 | 1.1025 |
| 026_831_00305170 | Lung | 831 | NCI-H2172 | 456 | 6.053261 | 0.837 | 0.856 | 0.908 | 0.8908 | 0.9397 | 0.905 | 0.9677 | 0.9512 | 0.9839 |
| 026_728_00305180 | Lung | 728 | NCI-H2196 | 456 | 6.171009 | 0.9 | 0.903 | 0.976 | 0.9979 | 0.9199 | 0.928 | 0.9453 | 0.9369 | 0.941 |
| 026_689_00300910 | Lung | 689 | NCI-H187 | 456 | 6.191556 | 0.887 | 1.009 | 0.976 | 0.9779 | 0.9535 | 1.047 | 1.021 | 1.0846 | 0.959 |
| 026_785_00302770 | Lung | 785 | SBC-1 | 456 | 6.234067 | 0.875 | 1.037 | 1.061 | 0.9376 | 1.0385 | 0.937 | 0.9403 | 1.0039 | 1.1602 |
| 026_8280_00306910 | Lung | 8280 | COR-L321 | 456 | 6.254083 | 0.965 | 0.937 | 1.019 | 0.8492 | 0.948 | 1.048 | 1.0366 | 1.0761 | 0.9586 |
| 026_706_00318720 | Lung | 706 | NCI-H1105 | 456 | 6.355 | 0.897 | 0.965 | 1.009 | 0.9821 | 0.9646 | 1.037 | 0.9974 | 0.9667 | 0.9589 |
| 026_712_00305150 | Lung | 712 | NCI-H1436 | 456 | 6.498513 | 0.959 | 0.818 | 0.938 | 1.1033 | 0.9924 | 0.922 | 1.0128 | 1.0035 | 0.9263 |
| 026_8079_00306720 | Lung | 8079 | IST-SL2 | 456 | 6.56418 | 0.947 | 0.972 | 0.935 | 0.9658 | 0.9445 | 0.966 | 0.9554 | 1.019 | 1.0006 |
| 026_743_00303220 | Lung | 743 | DMS 114 | 456 | 6.614771 | 0.862 | 0.853 | 0.785 | 0.7831 | 0.861 | 0.851 | 0.8815 | 0.9129 | 1.0374 |
| 026_8022_00306711 | Lung | 8022 | COLO-668 | 456 | 6.912523 | 0.97 | 0.968 | 0.956 | 1 | 0.9608 | 0.917 | 1.0093 | 0.9217 | 0.9308 |
| 026_8109_00308510 | Lung | 8109 | LU-134-A | 456 | 6.98132 | 0.924 | 1.012 | 1.105 | 0.9946 | 0.9684 | 1.081 | 0.9878 | 0.9397 | 0.9606 |
| 026_758_00303240 | Lung | 758 | HCC-33 | 456 | 7.022636 | 0.931 | 1.056 | 1.002 | 1.0822 | 1.0508 | 1.011 | 1.0081 | 1.1612 | 1.1932 |
| 026_771_00306940 | Lung | 771 | Lu-135 | 456 | 7.040802 | 0.988 | 0.969 | 0.972 | 1.0271 | 0.9989 | 1.006 | 0.993 | 1.0649 | 1.0235 |
| 026_694_00302790 | Lung | 694 | NCI-H196 | 456 | 7.041262 | 0.976 | 0.954 | 0.975 | 0.9548 | 0.9357 | 0.922 | 0.9564 | 0.9433 | 1.019 |
| 026_764_00308550 | Lung | 764 | COR-L95 | 456 | 7.251052 | 0.978 | 1.021 | 0.944 | 1.002 | 1.0708 | 0.947 | 0.9637 | 0.9383 | 0.903 |
| 026_763_00300900 | Lung | 763 | COR-L88 | 456 | 7.365052 | 0.96 | 1.168 | 0.891 | 1.1434 | 1.1503 | 0.976 | 1.0066 | 1.0636 | 1.052 |
| 026_8134_00306730 | Lung | 8134 | NCI-H64 | 456 | 7.782872 | 1.026 | 1.003 | 0.976 | 1.0025 | 0.951 | 1.047 | 0.9338 | 0.8997 | 1.0076 |
| 026_712_00311730 | Lung | 712 | NCI-H1436 | 456 | 7.804947 | 1.019 | 1.049 | 0.947 | 0.9403 | 0.9104 | 0.931 | 0.9349 | 0.9472 | 0.9659 |
| 026_8018_00304801 | Lung1NS | 8018 | Calu-6 | 456 | −0.899538 | 0.106 | 0.165 | 0.183 | 0.2075 | 0.2699 | 0.394 | 0.8182 | 0.9121 | 1.0531 |
| | CLC | | | | | | | | | | | | | |
| 026_1246_00304570 | Lung1NS | 1246 | NCI-H1770 | 456 | −0.072444 | 0.109 | 0.175 | 0.267 | 0.3543 | 0.4184 | 0.552 | 0.6475 | 1.0183 | 0.9838 |
| | CLC | | | | | | | | | | | | | |
| 026_847_00304580 | Lung1NS | 847 | NCI-H2087 | 456 | 1.048994 | 0.368 | 0.402 | 0.458 | 0.4756 | 0.5065 | 0.683 | 0.7888 | 0.9774 | 0.9846 |
| | CLC | | | | | | | | | | | | | |
| 026_680_00298830 | Lung1NS | 680 | NCI-H727 | 456 | 1.334143 | 0.329 | 0.434 | 0.475 | 0.4492 | 0.5064 | 0.604 | 0.8522 | 0.9134 | 0.98 |
| | CLC | | | | | | | | | | | | | |
| 026_748_00304590 | Lung1NS | 748 | NCI-H226 | 456 | 1.690128 | 0.342 | 0.457 | 0.514 | 0.5901 | 0.7687 | 0.757 | 0.7854 | 1.0039 | 0.932 |
| | CLC | | | | | | | | | | | | | |
| 026_851_00298380 | Lung1NS | 851 | CAL-12T | 456 | 1.708943 | 0.452 | 0.449 | 0.49 | 0.5839 | 0.7337 | 0.842 | 0.9205 | 0.938 | 0.9948 |
| | CLC | | | | | | | | | | | | | |
| 026_861_00300230 | Lung1NS | 861 | LCLC-97TM1 | 456 | 2.249036 | 0.424 | 0.573 | 0.622 | 0.7209 | 0.7517 | 0.873 | 1.0412 | 0.9724 | 0.974 |
| | CLC | | | | | | | | | | | | | |
| 026_1245_00304550 | Lung1NS | 1245 | NCI-H1648 | 456 | 2.511131 | 0.507 | 0.571 | 0.662 | 0.5623 | 0.7611 | 0.961 | 1.0696 | 1.0483 | 1.4204 |
| | CLC | | | | | | | | | | | | | |
| 026_1180_00308140 | Lung1NS | 1180 | NCI-H3122 | 456 | 2.550953 | 0.534 | 0.517 | 0.616 | 0.6044 | 0.7162 | 0.883 | 0.9512 | 0.9659 | 0.9657 |
| | CLC | | | | | | | | | | | | | |
| 026_802_00298451 | Lung1NS | 802 | NCI-H358 | 456 | 2.662533 | 0.509 | 0.53 | 0.626 | 0.7229 | 0.8072 | 0.806 | 0.8743 | 0.9218 | 0.9534 |
| | CLC | | | | | | | | | | | | | |
| 026_815_00311140 | Lung1NS | 815 | NCI-H1623 | 456 | 2.802368 | 0.523 | 0.592 | 0.685 | 0.6168 | 0.6264 | 0.746 | 0.872 | 0.8493 | 1.0805 |
| | CLC | | | | | | | | | | | | | |
| 026_1180_00302350 | Lung1NS | 1180 | NCI-H3122 | 456 | 2.945291 | 0.724 | 0.649 | 0.605 | 0.75 | 0.7455 | 0.913 | 0.9083 | 1.0538 | 1.1763 |
| | CLC | | | | | | | | | | | | | |
| 026_8040_00304501 | Lung1NS | 8040 | EKVX | 456 | 3.134434 | 0.532 | 0.642 | 0.782 | 0.752 | 0.7074 | 0.804 | 0.9087 | 1.0521 | 1.0032 |
| | CLC | | | | | | | | | | | | | |
| 026_865_00308451 | Lung1NS | 865 | COR-L23 | 456 | 3.225036 | 0.58 | 0.657 | 0.735 | 0.83 | 0.9489 | 0.935 | 1.0144 | 1.0355 | 1.0002 |
| | CLC | | | | | | | | | | | | | |
| 026_1243_00304541 | Lung1NS | 1243 | NCI-H1395 | 456 | 3.23207 | 0.553 | 0.739 | 0.944 | 0.948 | 0.9289 | 0.926 | 0.9919 | 1.0441 | 1.1113 |
| | CLC | | | | | | | | | | | | | |
| 026_884_00308160 | Lung1NS | 884 | RERF-LC-MS | 456 | 3.239905 | 0.585 | 0.663 | 0.662 | 0.6574 | 0.7423 | 0.886 | 0.9636 | 0.9621 | 0.9957 |
| | CLC | | | | | | | | | | | | | |
| 026_796_00295871 | Lung1NS | 796 | NCI-H2009 | 456 | 3.244878 | 0.582 | 0.62 | 0.631 | 0.635 | 0.7036 | 0.747 | 0.8586 | 0.9443 | 0.9943 |
| | CLC | | | | | | | | | | | | | |
| 026_799_00295880 | Lung1NS | 799 | NCI-H661 | 456 | 3.295179 | 0.667 | 0.587 | 0.685 | 0.6862 | 0.8151 | 0.782 | 1.0055 | 0.9053 | 0.9863 |
| | CLC | | | | | | | | | | | | | |
| 026_822_00311150 | Lung1NS | 822 | NCI-H1869 | 456 | 3.570833 | 0.153 | 0.716 | 0.736 | 0.7399 | 0.8095 | 0.819 | 1.026 | 0.9674 | 1.0055 |
| | CLC | | | | | | | | | | | | | |
| 026_756_00302670 | Lung1NS | 756 | BEN | 456 | 3.65632 | 0.639 | 0.833 | 0.942 | 0.9322 | 0.9128 | 0.965 | 0.9486 | 1.0116 | 1.0192 |
| | CLC | | | | | | | | | | | | | |
| 026_876_00299781 | Lung1NS | 876 | LU65 | 456 | 3.69068 | 0.635 | 0.695 | 0.704 | 0.7208 | 0.8211 | 0.878 | 0.9851 | 1.0211 | 1.0551 |
| | CLC | | | | | | | | | | | | | |
| 026_805_00304531 | Lung1NS | 805 | NCI-H1155 | 456 | 3.716538 | 0.61 | 0.788 | 0.787 | 0.8467 | 0.9573 | 0.91 | 0.953 | 1.0683 | 1.0508 |
| | CLC | | | | | | | | | | | | | |
| 026_834_00304610 | Lung1NS | 834 | NCI-H2347 | 456 | 3.72347 | 0.653 | 0.736 | 0.756 | 0.8026 | 0.8524 | 0.984 | 0.9516 | 1.2251 | 1.0554 |
| | CLC | | | | | | | | | | | | | |
| 026_822_00304840 | Lung1NS | 822 | NCI-H1869 | 456 | 3.749478 | 0.594 | 0.736 | 0.791 | 0.7802 | 0.8592 | 0.824 | 0.8872 | 0.9703 | 1.0592 |
| | CLC | | | | | | | | | | | | | |
| 026_835_00302390 | Lung1NS | 835 | NCI-H2405 | 456 | 3.789193 | 0.693 | 0.659 | 0.651 | 0.6574 | 0.7337 | 0.768 | 0.8849 | 0.9046 | 1.0375 |
| | CLC | | | | | | | | | | | | | |
| 026_678_00304981 | Lung1NS | 678 | UMC-11 | 456 | 3.815948 | 0.655 | 0.751 | 0.848 | 0.8851 | 0.9411 | 1.013 | 0.9806 | 1.0842 | 1.1132 |
| | CLC | | | | | | | | | | | | | |
| 026_807_00314280 | Lung1NS | 807 | NCI-H650 | 456 | 3.884087 | 0.604 | 0.778 | 0.772 | 0.8577 | 0.8346 | 0.815 | 1.0068 | 0.831 | 0.9772 |
| | CLC | | | | | | | | | | | | | |
| 026_871_00299771 | Lung1NS | 871 | LK-2 | 456 | 3.898613 | 0.684 | 0.806 | 0.903 | 0.9071 | 0.958 | 1.018 | 0.9981 | 1.0604 | 1.0786 |
| | CLC | | | | | | | | | | | | | |
| 026_1249_00308150 | Lung1NS | 1249 | NCI-H720 | 456 | 3.907633 | 0.632 | 0.829 | 0.932 | 1.0847 | 1.526 | 0.799 | 0.8751 | 1.2042 | 1.1215 |
| | CLC | | | | | | | | | | | | | |
| 026_8231_00304510 | Lung1NS | 8231 | EMC-BAC-1 | 456 | 3.943946 | 0.675 | 0.713 | 0.751 | 0.8781 | 0.831 | 0.931 | 0.976 | 1.0569 | 1.0459 |
| | CLC | | | | | | | | | | | | | |
| 026_820_00304560 | Lung1NS | 820 | NCI-H1755 | 456 | 3.964037 | 0.658 | 0.769 | 0.785 | 0.8744 | 0.8799 | 0.931 | 0.9563 | 1.1261 | 1.0206 |
| | CLC | | | | | | | | | | | | | |
| 026_815_00304830 | Lung1NS | 815 | NCI-H1623 | 456 | 3.981628 | 0.646 | 0.709 | 0.712 | 0.7108 | 0.8124 | 0.879 | 0.7844 | 1.0245 | 1.0786 |
| | CLC | | | | | | | | | | | | | |
| 026_839_00302410 | Lung1NS | 839 | SW 900 | 456 | 3.982367 | 0.65 | 0.725 | 0.782 | 0.8242 | 0.8576 | 0.897 | 0.9535 | 1.0086 | 0.9978 |
| | CLC | | | | | | | | | | | | | |
| 026_804_00308480 | Lung1NS | 804 | NCI-H810 | 456 | 4.03318 | 0.731 | 0.763 | 0.921 | 0.9442 | 0.9087 | 1.003 | 0.9482 | 0.9716 | 1.0139 |
| 1333 | CLC | | | | | | | | | | | | | |
| 026_678_00309011 | Lung1NS | 678 | UMC-11 | 456 | 4.0744 | 0.514 | 0.846 | 0.86 | 0.931 | 0.9037 | 0.925 | 0.9312 | 0.9354 | 1.0786 |
| | CLC | | | | | | | | | | | | | |
| 026_842_00298540 | Lung1NS | 842 | NCI-H520 | 456 | 4.082969 | 0.729 | 0.811 | 0.916 | 1.0516 | 1.0142 | 1.002 | 1.0457 | 1.0025 | 0.9761 |
| | CLC | | | | | | | | | | | | | |
| 026_824_00314260 | Lung1NS | 824 | NCI-H1944 | 456 | 4.107799 | 0.695 | 0.731 | 0.691 | 0.7704 | 0.893 | 0.886 | 0.905 | 0.9791 | 1.0866 |
| | CLC | | | | | | | | | | | | | |
| 026_888_00298370 | Lung1NS | 888 | ABC-1 | 456 | 4.123019 | 0.64 | 0.676 | 0.717 | 0.7343 | 0.8328 | 0.84 | 0.8774 | 0.9195 | 0.9448 |
| | CLC | | | | | | | | | | | | | |
| 026_823_00298430 | Lung1NS | 823 | NCI-H1915 | 456 | 4.260241 | 0.691 | 0.707 | 0.702 | 0.7393 | 0.7957 | 0.834 | 0.9053 | 0.9612 | 0.9902 |
| | CLC | | | | | | | | | | | | | |
| 026_8232_00304520 | Lung1NS | 8232 | EMC-BAC-2 | 456 | 4.327709 | 0.712 | 0.795 | 0.825 | 0.8558 | 0.8815 | 0.959 | 0.9014 | 1.1842 | 1.0135 |
| | CLC | | | | | | | | | | | | | |
| 026_755_00300611 | Lung1NS | 755 | NCI-H1975 | 456 | 4.450033 | 0.666 | 0.968 | 0.963 | 0.9052 | 0.9346 | 0.928 | 0.9969 | 0.9681 | 0.973 |
| | CLC | | | | | | | | | | | | | |
| 026_868_00295440 | Lung1NS | 868 | PC-14 | 456 | 4.458594 | 0.726 | 0.893 | 0.842 | 0.8971 | 0.9361 | 1.057 | 0.9146 | 1.1281 | 0.933 |
| | CLC | | | | | | | | | | | | | |
| 026_872_00299750 | Lung1NS | 872 | HARA | 456 | 4.480998 | 0.731 | 0.742 | 0.798 | 0.8152 | 0.8972 | 0.941 | 0.9735 | 1.0074 | 1.0301 |
| | CLC | | | | | | | | | | | | | |
| 026_800_00298441 | Lung1NS | 800 | NCI-H23 | 456 | 4.511394 | 0.702 | 0.698 | 0.786 | 0.7938 | 0.8549 | 0.889 | 0.9216 | 0.9447 | 1.013 |
| | CLC | | | | | | | | | | | | | |
| 026_836_00304620 | Lung1NS | 836 | NCI-H2444 | 456 | 4.530511 | 0.684 | 0.798 | 0.819 | 0.8507 | 0.863 | 0.86 | 0.9116 | 1.0106 | 1.0518 |
| | CLC | | | | | | | | | | | | | |
| 026_865_00296401 | Lung1NS | 865 | COR-L23 | 456 | 4.577187 | 0.791 | 0.78 | 0.815 | 0.8605 | 0.9148 | 0.924 | 1.1179 | 1.1287 | 1.1293 |
| | CLC | | | | | | | | | | | | | |
| 026_858_00300591 | Lung1NS | 858 | HCC-78 | 456 | 4.583505 | 0.703 | 0.983 | 0.936 | 1.06 | 0.9813 | 0.96 | 1.0028 | 0.9854 | 1.0584 |
| | CLC | | | | | | | | | | | | | |
| 026_854_00300681 | Lung1NS | 854 | EPLC-272H | 456 | 4.590654 | 0.736 | 0.914 | 0.943 | 1.0019 | 1.0017 | 0.864 | 0.8937 | 0.9686 | 1.0373 |
| | CLC | | | | | | | | | | | | | |
| 026_837_00311160 | Lung1NS | 837 | NCI-H2122 | 456 | 4.595192 | 0.771 | 0.917 | 0.969 | 0.9878 | 0.9667 | 1.017 | 1.0165 | 1.0062 | 1.0324 |
| | CLC | | | | | | | | | | | | | |
| 026_8111_00308110 | Lung1NS | 8111 | LXF-289 | 456 | 4.689278 | 0.711 | 0.796 | 0.884 | 0.8648 | 0.868 | 0.89 | 0.9108 | 0.8979 | 0.9642 |
| | CLC | | | | | | | | | | | | | |
| 026_1136_00308471 | Lung1NS | 1136 | NCI-H1993 | 456 | 4.705234 | 0.725 | 0.768 | 0.682 | 0.7469 | 0.7866 | 0.883 | 0.9296 | 0.914 | 0.9652 |
| | CLC | | | | | | | | | | | | | |
| 026_827_00308860 | Lung1NS | 827 | NCI-H2085 | 456 | 4.711775 | 0.79 | 0.823 | 0.87 | 0.9311 | 0.924 | 1.007 | 0.967 | 1.0124 | 1.1093 |
| | CLC | | | | | | | | | | | | | |
| 026_859_00311090 | Lung1NS | 859 | HCC-827 | 456 | 4.735253 | 0.737 | 0.86 | 0.852 | 0.7863 | 0.8298 | 0.853 | 1.0366 | 1.1949 | 0.9783 |
| | CLC | | | | | | | | | | | | | |
| 026_886_00296261 | Lung1NS | 886 | EBC-1 | 456 | 4.744821 | 0.761 | 0.848 | 0.884 | 0.9053 | 0.937 | 0.969 | 0.9903 | 0.9432 | 1.0947 |
| | CLC | | | | | | | | | | | | | |
| 026_8132_00308130 | Lung1NS | 8132 | NCI-H2126 | 456 | 4.781635 | 0.749 | 0.796 | 0.839 | 0.8331 | 0.8916 | 0.939 | 0.9049 | 0.9661 | 0.9286 |
| | CLC | | | | | | | | | | | | | |
| 026_793_00299711 | Lung1NS | 793 | NCI-H1781 | 456 | 4.904802 | 0.775 | 0.801 | 0.686 | 0.7429 | 0.7804 | 0.897 | 0.9136 | 0.9608 | 1.0112 |
| | CLC | | | | | | | | | | | | | |
| 026_1247_00314271 | Lung1NS | 1247 | NCI-H2291 | 456 | 4.945975 | 0.773 | 0.893 | 0.836 | 0.831 | 0.851 | 1.029 | 1.0615 | 0.9828 | 1.0493 |
| | CLC | | | | | | | | | | | | | |
| 026_860_00298400 | Lung1NS | 860 | LCLC-103H | 456 | 4.97209 | 0.751 | 0.819 | 0.803 | 0.8985 | 0.8725 | 0.898 | 0.9606 | 0.9519 | 1.061 |
| | CLC | | | | | | | | | | | | | |
| 026_806_00300270 | Lung1NS | 806 | NCI-H647 | 456 | 5.044917 | 0.792 | 0.845 | 0.882 | 0.8698 | 0.8856 | 0.881 | 1.0769 | 0.9333 | 1.0648 |
| | CLC | | | | | | | | | | | | | |
| 026_877_00300630 | Lung1NS | 877 | PC-3 | 456 | 5.056812 | 0.847 | 0.832 | 0.968 | 0.9943 | 1.0985 | 1.01 | 1.0152 | 1.0063 | 1.1608 |
| | CLC | | [JPC-3] | | | | | | | | | | | |
| 026_753_00298460 | Lung1NS | 753 | NCI-H460 | 456 | 5.061076 | 0.839 | 0.888 | 0.945 | 0.9015 | 1.041 | 1.039 | 1.021 | 0.9905 | 1.0126 |
| | CLC | | | | | | | | | | | | | |
| 026_844_00295461 | Lung1NS | 844 | SW 1573 | 456 | 5.077017 | 0.798 | 0.834 | 0.833 | 0.8824 | 0.8946 | 0.971 | 1.0223 | 0.9943 | 1.0074 |
| | CLC | | | | | | | | | | | | | |
| 026_8088_00314320 | Lung1NS | 8088 | KNS-62 | 456 | 5.130485 | 0.86 | 0.847 | 0.879 | 0.9874 | 0.9738 | 1.047 | 1.0602 | 1.0303 | 0.9876 |
| | CLC | | | | | | | | | | | | | |
| 026_848_00300641 | Lung1NS | 848 | SK-LU-1 | 456 | 5.2771 | 0.811 | 0.91 | 0.946 | 0.9174 | 0.9429 | 0.922 | 0.9544 | 1.0372 | 1.0115 |
| | CLC | | | | | | | | | | | | | |
| 026_864_00304961 | Lung1NS | 864 | COR-L105 | 456 | 5.319498 | 0.718 | 0.78 | 0.762 | 0.8018 | 0.7683 | 0.837 | 0.9721 | 0.8644 | 0.9527 |
| | CLC | | | | | | | | | | | | | |
| 026_677_00298361 | Lung1NS | 677 | A549 | 456 | 5.414187 | 0.763 | 0.799 | 0.843 | 0.8595 | 0.9001 | 0.919 | 0.9405 | 0.9783 | 0.996 |
| | CLC | | | | | | | | | | | | | |
| 026_8207_00304810 | Lung1NS | 8207 | LC-1F | 456 | 5.423288 | 0.825 | 0.794 | 0.845 | 0.8457 | 0.8483 | 0.976 | 0.8421 | 0.8626 | 0.9602 |
| | CLC | | | | | | | | | | | | | |
| 026_833_00299790 | Lung1NS | 833 | NCI-H2342 | 456 | 5.548934 | 0.856 | 0.888 | 0.917 | 0.9241 | 0.9293 | 1.015 | 0.9449 | 1.0616 | 0.9624 |
| | CLC | | | | | | | | | | | | | |
| 026_870_00299801 | Lung1NS | 870 | RERF-LC-KJ | 456 | 5.685511 | 0.892 | 0.96 | 0.999 | 0.9623 | 1.0469 | 1.054 | 1.0305 | 1.0128 | 1.0476 |
| | CLC | | | | | | | | | | | | | |
| 026_845_00311341 | Lung1NS | 845 | NCI-H1838 | 456 | 5.711103 | 0.847 | 0.867 | 0.909 | 0.9088 | 0.9208 | 0.932 | 0.9775 | 0.9857 | 0.9755 |
| | CLC | | | | | | | | | | | | | |
| 026_8103_00306210 | Lung1NS | 8103 | LC-2-ad | 456 | 5.718708 | 0.836 | 0.96 | 1.004 | 0.9427 | 1.0257 | 1.064 | 1.0682 | 0.9834 | 1.007 |
| | CLC | | | | | | | | | | | | | |
| 026_794_00300601 | Lung1NS | 794 | NCI-H1792 | 456 | 5.721238 | 0.339 | 0.904 | 0.992 | 0.9248 | 0.9615 | 1.005 | 0.9405 | 0.9595 | 1.0225 |
| | CLC | | | | | | | | | | | | | |
| 026_890_00300220 | Lung1NS | 890 | H3255 | 456 | 5.722728 | 0.903 | 0.862 | 0.844 | 0.9926 | 1.005 | 0.998 | 1.1503 | 1.1306 | 1.1501 |
| | CLC | | | | | | | | | | | | | |
| 026_862_00304820 | Lung1NS | 862 | LOU-NH91 | 456 | 5.738178 | 0.886 | 0.864 | 0.837 | 0.9386 | 0.9258 | 0.887 | 0.948 | 1.0561 | 1.1752 |
| | CLC | | | | | | | | | | | | | |
| 026_812_00298410 | Lung1NS | 812 | NCI-H1437 | 456 | 5.791603 | 0.894 | 0.946 | 0.993 | 0.9231 | 0.9704 | 1.003 | 1.0591 | 1.0305 | 1.0361 |
| | CLC | | | | | | | | | | | | | |
| 026_862_00316560 | Lung1NS | 862 | LOU-NH91 | 456 | 5.867157 | 0.871 | 0.905 | 0.923 | 0.9415 | 0.9143 | 0.955 | 0.9491 | 0.9721 | 0.9923 |
| | CLC | | | | | | | | | | | | | |
| 026_857_00296270 | Lung1NS | 857 | HCC-44 | 456 | 5.930045 | 0.904 | 0.868 | 0.893 | 0.9249 | 0.9348 | 0.956 | 1.0259 | 0.9727 | 1.0027 |
| | CLC | | | | | | | | | | | | | |
| 026_679_00296240 | Lung1NS | 679 | ChaGo-K-1 | 456 | 5.965866 | 0.878 | 0.915 | 0.949 | 0.8968 | 0.942 | 0.953 | 0.9732 | 0.9711 | 1.0338 |
| | CLC | | | | | | | | | | | | | |
| 026_791_00298421 | Lung1NS | 791 | NCI-H1650 | 456 | 5.971987 | 0.888 | 0.896 | 0.979 | 0.9429 | 0.9543 | 0.921 | 0.9516 | 0.9551 | 0.9966 |
| | CLC | | | | | | | | | | | | | |
| 026_864_00311551 | Lung1NS | 864 | COR-L105 | 456 | 5.975297 | 0.765 | 0.838 | 0.843 | 0.8384 | 0.8225 | 0.848 | 0.8917 | 0.8749 | 0.9811 |
| | CLC | | | | | | | | | | | | | |
| 026_846_00300620 | Lung1NS | 846 | NCI-H2030 | 456 | 6.014339 | 0.886 | 0.979 | 0.985 | 0.9316 | 1.0428 | 0.925 | 0.9778 | 1.056 | 0.9904 |
| | CLC | | | | | | | | | | | | | |
| 026_841_00296291 | Lung1NS | 841 | NCI-H2170 | 456 | 6.01763 | 0.897 | 0.938 | 0.928 | 0.9356 | 0.9557 | 0.955 | 1.0968 | 1.0708 | 1.0963 |
| | CLC | | | | | | | | | | | | | |
| 026_813_00308231 | Lung1NS | 813 | NCI-H1563 | 456 | 6.06364 | 0.856 | 0.979 | 0.977 | 0.9369 | 0.9242 | 0.912 | 0.8859 | 0.9059 | 0.8856 |
| | CLC | | | | | | | | | | | | | |
| 026_816_00295860 | Lung1NS | 816 | NCI-H1651 | 456 | 6.160326 | 0.864 | 1.039 | 1.026 | 1.008 | 1.0776 | 0.871 | 1.0553 | 1.0199 | 0.9978 |
| | CLC | | | | | | | | | | | | | |
| 026_818_00308241 | Lung1NS | 818 | NCI-H1703 | 456 | 6.172519 | 0.946 | 0.829 | 0.858 | 0.9042 | 0.9197 | 0.887 | 0.9097 | 0.9729 | 0.9634 |
| | CLC | | | | | | | | | | | | | |
| 026_808_00296300 | Lung1NS | 808 | NCI-H838 | 456 | 6.172929 | 0.922 | 0.941 | 0.955 | 1.022 | 0.964 | 1.043 | 1.0358 | 1.0134 | 1.0658 |
| | CLC | | | | | | | | | | | | | |
| 026_855_00302330 | Lung1NS | 855 | HCC-15 | 456 | 6.195295 | 0.868 | 0.911 | 0.901 | 0.8895 | 0.9071 | 0.95 | 0.9608 | 1.0091 | 1.0383 |
| | CLC | | | | | | | | | | | | | |
| 026_803_00298470 | Lung1NS | 803 | NCI-H522 | 456 | 6.200929 | 0.878 | 0.933 | 0.953 | 0.8695 | 0.9015 | 0.833 | 0.8882 | 0.8789 | 1.1066 |
| | CLC | | | | | | | | | | | | | |
| 026_832_00303110 | Lung1NS | 832 | NCI-H2228 | 456 | 6.240586 | 0.908 | 0.872 | 0.907 | 0.9005 | 0.9724 | 0.974 | 0.9776 | 0.9706 | 0.968 |
| | CLC | | | | | | | | | | | | | |
| 026_874_00308171 | Lung1NS | 874 | RERF- | 456 | 6.282697 | 1.064 | 0.854 | 0.829 | 0.8009 | 0.8914 | 0.943 | 0.8612 | 0.9931 | 0.9393 |
| | CLC | | LC-Sq1 | | | | | | | | | | | |
| 026_879_00308101 | Lung1NS | 879 | LU99A | 456 | 6.327465 | 0.881 | 0.987 | 0.995 | 1.0199 | 0.9725 | 0.948 | 0.9588 | 0.9756 | 0.9692 |
| | CLC | | | | | | | | | | | | | |
| 026_856_00299760 | Lung1NS | 856 | HCC-366 | 456 | 6.328052 | 0.936 | 0.943 | 0.959 | 1.0286 | 0.9991 | 1.002 | 1.0072 | 1.0077 | 1.0015 |
| | CLC | | | | | | | | | | | | | |
| 026_798_00304790 | Lung1NS | 798 | Calu-3 | 456 | 6.338698 | 0.926 | 0.986 | 0.869 | 0.9635 | 1.0683 | 1.166 | 1.1053 | 1.1123 | 1.1003 |
| | CLC | | | | | | | | | | | | | |
| 026_825_00308250 | Lung1NS | 825 | NCI-H2023 | 456 | 6.435057 | 0.938 | 0.885 | 0.929 | 0.961 | 0.9166 | 0.959 | 0.9502 | 0.9835 | 0.9846 |
| | CLC | | | | | | | | | | | | | |
| 026_8072_00306200 | Lung1NS | 8072 | HOP-62 | 456 | 6.481774 | 0.973 | 0.958 | 0.931 | 0.9405 | 0.9799 | 0.901 | 1.0374 | 0.9717 | 1.1512 |
| | CLC | | | | | | | | | | | | | |
| 026_843_00296331 | Lung1NS | 843 | SK-MES-1 | 456 | 6.499026 | 0.91 | 0.99 | 0.934 | 0.9509 | 0.9694 | 1.003 | 0.9804 | 0.9674 | 1.0295 |
| | CLC | | | | | | | | | | | | | |
| 026_1251_00318710 | Lung1NS | 1251 | NCI-H835 | 456 | 6.684349 | 0.964 | 1.054 | 0.854 | 0.9853 | 0.77 | 0.963 | 0.8714 | 0.9637 | 1.1416 |
| | CLC | | | | | | | | | | | | | |
| 026_816_00302370 | Lung1NS | 816 | NCI-H1651 | 456 | 6.769433 | 0.903 | 1.072 | 1.021 | 1.002 | 1.0583 | 0.94 | 1.0664 | 1.0462 | 1.0774 |
| | CLC | | | | | | | | | | | | | |
| 026_801_00295941 | Lung1NS | 801 | NCI-H1299 | 456 | 6.808075 | 1.036 | 0.898 | 0.912 | 0.9194 | 0.9832 | 0.955 | 1.0252 | 0.9946 | 1.085 |
| | CLC | | | | | | | | | | | | | |
| 026_850_00311050 | Lung1NS | 850 | 201T | 456 | 6.874432 | 0.942 | 0.957 | 0.988 | 0.989 | 0.9596 | 0.985 | 0.9759 | 0.9839 | 0.9726 |
| | CLC | | | | | | | | | | | | | |
| 026_818_00309001 | Lung1NS | 818 | NCI-H1703 | 456 | 7.192409 | 0.889 | 0.857 | 0.855 | 0.8355 | 0.8207 | 0.856 | 0.8191 | 0.9124 | 0.9468 |
| | CLC | | | | | | | | | | | | | |
| 026_1247_00304851 | Lung1NS | 1247 | NCI-H2291 | 456 | 7.252153 | 1.162 | 1.047 | 0.877 | 1.034 | 0.8759 | 0.694 | 1.2149 | 1.0391 | 1.1021 |
| | CLC | | | | | | | | | | | | | |
| 026_819_00306250 | Lung1NS | 819 | NCI-H1734 | 456 | 7.342705 | 1.046 | 1.007 | 1.012 | 1.018 | 0.9902 | 0.945 | 0.9742 | 0.9718 | 1.0221 |
| | CLC | | | | | | | | | | | | | |
| 026_821_00306261 | Lung1NS | 821 | NCI-H1793 | 456 | 7.345651 | 1.015 | 1.1 | 1.058 | 1.0171 | 1.0121 | 1.065 | 1.0342 | 1.0776 | 1.0468 |
| | CLC | | | | | | | | | | | | | |
| 026_798_00308080 | Lung1NS | 798 | Calu-3 | 456 | 7.389624 | 1.032 | 1 | 0.914 | 0.9927 | 1.0342 | 1.004 | 1.0095 | 0.9327 | 0.938 |
| | CLC | | | | | | | | | | | | | |
| 026_678_00306181 | Lung1NS | 678 | UMC-11 | 456 | 7.458364 | 1.03 | 1.058 | 1.054 | 1.0958 | 1.0021 | 1.03 | 1.0784 | 1.0779 | 1.0688 |
| | CLC | | | | | | | | | | | | | |
| 026_797_00308730 | Lung1NS | 797 | NCI-H596 | 456 | 7.556662 | 1.124 | 1.076 | 0.92 | 0.9992 | 0.9285 | 0.936 | 0.911 | 1.0276 | 0.8862 |
| | CLC | | | | | | | | | | | | | |
| 026_790_00306231 | Lung1NS | 790 | NCI-H1573 | 456 | 7.606756 | 1.049 | 1 | 1.054 | 1.0146 | 1.0291 | 0.99 | 1.0304 | 1.0221 | 1.0512 |
| | CLC | | | | | | | | | | | | | |
| 026_752_00306191 | Lung1NS | 752 | A-427 | 456 | 7.608444 | 1.109 | 0.988 | 1.054 | 1.0224 | 0.9429 | 1.084 | 1.0956 | 1.0273 | 1.0528 |
| | CLC | | | | | | | | | | | | | |
| 026_845_00306271 | Lung1NS | 845 | NCI-H1838 | 456 | 7.666847 | 1.074 | 1.08 | 1.064 | 1.0779 | 1.0805 | 1.046 | 1.0674 | 0.9941 | 1.02 |
| | CLC | | | | | | | | | | | | | |
| 026_8133_00306280 | Lung1NS | 8133 | NCI-H322M | 456 | 7.713112 | 1.158 | 1.06 | 1.135 | 1.1499 | 1.1305 | 1.103 | 1.1464 | 1.1475 | 1.0913 |
| | CLC | | | | | | | | | | | | | |
| 026_8130_00306220 | Lung1NS | 8130 | NCI-H1355 | 456 | 7.765888 | 1.12 | 1.188 | 1.002 | 0.9001 | 0.8103 | 1.095 | 0.9608 | 0.9714 | 0.9997 |
| | CLC | | | | | | | | | | | | | |
| 026_683_00306241 | Lung1NS | 683 | NCI-H1581 | 456 | 7.774034 | 1.163 | 1.024 | 0.949 | 1.0959 | 1.0605 | 1.114 | 1.0118 | 0.9537 | 0.9212 |
| | CLC | | | | | | | | | | | | | |
| 026_792_00306140 | Lung1NS | 792 | NCI-H1666 | 456 | 7.825548 | 1.136 | 1.069 | 1.132 | 1.0901 | 1.0688 | 1.097 | 1.0684 | 1.0463 | 1.1115 |
| | CLC | | | | | | | | | | | | | |
| 026_840_00306151 | Lung1NS | 840 | NCI-H441 | 456 | 7.946249 | 1.088 | 1.059 | 1.052 | 1.0622 | 1.0476 | 1.049 | 1.0439 | 1.045 | 1.0714 |
| | CLC | | | | | | | | | | | | | |
| 026_8075_00306131 | Lung1NS | 8075 | IA-LM | 456 | 7.984543 | 1.046 | 1.042 | 1.043 | 1.0424 | 1.0323 | 1.048 | 1.0441 | 1.0279 | 1.0694 |
| | CLC | | | | | | | | | | | | | |
| 026_61_00285570 | Lymphoma | 61 | JSC-1 | 456 | 0.316646 | 0.313 | 0.329 | 0.341 | 0.3908 | 0.5654 | 0.63 | 0.724 | 0.8346 | 0.8527 |
| 026_8222_00291350 | Lymphoma | 8222 | H9 | 456 | 0.402947 | 0.102 | 0.284 | 0.272 | 0.3996 | 0.5762 | 0.732 | 0.8412 | 0.8346 | 0.8529 |
| 026_140_00291320 | Lymphoma | 140 | A3/KAW | 456 | 0.717935 | 0.185 | 0.296 | 0.408 | 0.4762 | 0.5561 | 0.736 | 0.8297 | 0.8761 | 0.8425 |
| 026_237_00291380 | Lymphoma | 237 | SU-DHL-16 | 456 | 0.982467 | 0.155 | 0.278 | 0.405 | 0.5737 | 0.6759 | 0.816 | 0.9129 | 0.9363 | 0.9318 |
| 026_220_00288720 | Lymphoma | 220 | OCI-LY-19 | 456 | 1.28484 | 0.324 | 0.393 | 0.455 | 0.5338 | 0.6443 | 0.826 | 0.9343 | 0.9969 | 1.073 |
| 026_257_00285640 | Lymphoma | 257 | WIL2 NS | 456 | 1.523653 | 0.32 | 0.456 | 0.509 | 0.6199 | 0.6926 | 0.771 | 0.8518 | 0.9524 | 0.934 |
| 026_239_00288750 | Lymphoma | 239 | SU-DHL-5 | 456 | 1.616577 | 0.174 | 0.577 | 0.461 | 0.7856 | 0.7368 | 0.845 | 0.9512 | 0.9535 | 1.2005 |
| 026_124_00287850 | Lymphoma | 124 | BC-1 | 456 | 1.816766 | 0.239 | 0.53 | 0.6 | 0.6778 | 0.8612 | 0.946 | 0.9754 | 0.9846 | 1.0038 |
| 026_104_00287960 | Lymphoma | 104 | TUR | 456 | 2.217781 | 0.396 | 0.51 | 0.597 | 0.7468 | 0.9062 | 0.94 | 0.9822 | 0.9761 | 1.0165 |
| 026_8199_00291330 | Lymphoma | 8199 | CTB-1 | 456 | 2.341488 | 0.445 | 0.509 | 0.654 | 0.6993 | 0.7792 | 0.767 | 0.8214 | 0.8543 | 0.9676 |
| 026_69_00283480 | Lymphoma | 69 | CA46 | 456 | 2.43077 | 0.358 | 0.6 | 0.775 | 0.78 | 0.8817 | 0.896 | 0.9912 | 0.9446 | 0.9358 |
| 026_112_00285620 | Lymphoma | 112 | SR | 456 | 2.466522 | 0.224 | 0.573 | 0.653 | 0.7668 | 0.8482 | 0.866 | 0.9087 | 0.9409 | 0.9255 |
| 026_241_00288760 | Lymphoma | 241 | SU-DHL-8 | 456 | 2.509783 | 0.398 | 0.583 | 0.743 | 0.8249 | 0.935 | 0.956 | 1.0439 | 0.9903 | 1.0527 |
| 026_255_00285610 | Lymphoma | 255 | Sci-1 | 456 | 2.705483 | 0.445 | 0.61 | 0.788 | 0.9473 | 0.9153 | 1.003 | 0.9035 | 0.8642 | 0.8757 |
| 026_62_00293930 | Lymphoma | 62 | IM-9 | 456 | 2.809638 | 0.572 | 0.562 | 0.594 | 0.6839 | 0.7773 | 1.18 | 0.876 | 1.0089 | 1.0329 |
| 026_93_00287880 | Lymphoma | 93 | HH | 456 | 2.828886 | 0.523 | 0.575 | 0.731 | 0.804 | 0.9283 | 0.948 | 0.9965 | 0.999 | 1.0069 |
| 026_216_00290710 | Lymphoma | 216 | NU-DUL-1 | 456 | 2.880782 | 0.459 | 0.664 | 0.767 | 0.8093 | 0.7784 | 0.884 | 0.9338 | 0.9363 | 0.9519 |
| 026_123_00287860 | Lymphoma | 123 | BC-3 | 456 | 2.893681 | 0.495 | 0.678 | 0.776 | 0.9051 | 1.0108 | 1.003 | 1.0056 | 0.9841 | 0.9941 |
| 026_113_00288520 | Lymphoma | 113 | DB | 456 | 2.901204 | 0.479 | 0.705 | 0.868 | 0.9097 | 0.9559 | 0.984 | 1.0104 | 0.992 | 1.0316 |
| 026_8035_00303290 | Lymphoma | 8035 | DOHH-2 | 456 | 2.927461 | 0.508 | 0.78 | 0.831 | 0.8009 | 0.9998 | 0.997 | 1.0295 | 0.9393 | 1.0203 |
| 026_240_00300290 | Lymphoma | 240 | SU-DHL-6 | 456 | 2.933827 | 0.208 | 0.663 | 0.733 | 0.7473 | 0.7814 | 0.839 | 0.8554 | 0.9327 | 1.029 |
| 026_248_00285630 | Lymphoma | 248 | VAL | 456 | 2.964017 | 0.528 | 0.639 | 0.739 | 0.9112 | 0.9265 | 0.96 | 1.0023 | 0.9827 | 0.9859 |
| 026_162_00290790 | Lymphoma | 162 | HDLM-2 | 456 | 2.980685 | 0.56 | 0.811 | 0.678 | 0.7341 | 0.7627 | 0.782 | 0.7915 | 0.9638 | 0.7387 |
| 026_105_00287940 | Lymphoma | 105 | RPMI 6666 | 456 | 3.064381 | 0.516 | 0.701 | 0.691 | 0.7257 | 0.8234 | 0.904 | 0.9883 | 1.0013 | 1.033 |
| 026_240_00302920 | Lymphoma | 240 | SU-DHL-6 | 456 | 3.114948 | 0.326 | 0.629 | 0.741 | 0.7802 | 0.8119 | 0.831 | 0.9347 | 0.921 | 0.9838 |
| 026_163_00287730 | Lymphoma | 163 | HD-MY-Z | 456 | 3.123344 | 0.582 | 0.606 | 0.654 | 0.6825 | 0.785 | 0.896 | 0.9395 | 0.98 | 1.0207 |
| 026_139_00283530 | Lymphoma | 139 | MC116 | 456 | 3.12577 | 0.085 | 0.686 | 0.917 | 0.9565 | 0.9611 | 0.99 | 0.9875 | 0.9789 | 0.9559 |
| 026_133_00293941 | Lymphoma | 133 | NK-92MI | 456 | 3.297366 | 0.637 | 0.637 | 0.757 | 0.8911 | 0.8633 | 1.136 | 1.0548 | 0.89 | 0.9001 |
| 026_282_00287750 | Lymphoma | 282 | P32/ISH | 456 | 3.328203 | 0.416 | 0.744 | 0.875 | 0.8947 | 0.8754 | 0.907 | 0.9959 | 0.9144 | 0.9277 |
| 026_80_00287950 | Lymphoma | 80 | ST486 | 456 | 3.520498 | 0.263 | 0.688 | 0.699 | 0.7969 | 0.8071 | 0.904 | 0.8902 | 0.9142 | 1.017 |
| 026_73_00283500 | Lymphoma | 73 | EB-3 | 456 | 3.528971 | 0.566 | 0.739 | 0.806 | 0.8214 | 0.8547 | 1.09 | 0.8321 | 0.8619 | 0.8548 |
| 026_60_00287891 | Lymphoma | 60 | JM1 | 456 | 3.618767 | 0.577 | 0.678 | 0.734 | 0.7932 | 0.8209 | 0.833 | 0.8444 | 0.8567 | 0.902 |
| 026_185_00288820 | Lymphoma | 185 | L-540 | 456 | 3.666174 | 0.626 | 0.88 | 0.936 | 1.0302 | 1.0245 | 1.037 | 1.0524 | 1.0175 | 1.0525 |
| 026_70_00283490 | Lymphoma | 70 | Daudi | 456 | 3.687192 | 0.603 | 0.789 | 0.925 | 0.8867 | 0.9929 | 0.987 | 0.8586 | 0.9555 | 0.8243 |
| 026_228_00291370 | Lymphoma | 228 | RC-K8 | 456 | 3.710403 | 0.605 | 0.678 | 0.732 | 0.7776 | 0.8387 | 0.943 | 0.9437 | 0.9368 | 0.9149 |
| 026_74_00285600 | Lymphoma | 74 | Raji | 456 | 3.736373 | 0.639 | 0.768 | 0.89 | 0.8258 | 0.9671 | 0.97 | 0.9444 | 0.9665 | 1.046 |
| 026_173_00288920 | Lymphoma | 173 | KARPAS-422 | 456 | 3.812903 | 0.638 | 0.787 | 0.876 | 0.8937 | 0.8652 | 0.959 | 0.9976 | 0.9896 | 1.005 |
| 026_125_00287910 | Lymphoma | 125 | MC/CAR | 456 | 3.860302 | 0.687 | 0.792 | 0.873 | 0.8871 | 1.009 | 1.011 | 0.9976 | 0.987 | 1.0162 |
| 026_280_00285420 | Lymphoma | 280 | SCC-3 | 456 | 3.903427 | 0.648 | 0.86 | 0.895 | 0.9301 | 0.9335 | 0.934 | 1.0707 | 0.9 | 0.9445 |
| 026_242_00288770 | Lymphoma | 242 | SUP-HD1 | 456 | 4.023177 | 0.671 | 0.815 | 0.875 | 0.8573 | 0.8914 | 0.965 | 1.0137 | 0.9754 | 1.1043 |
| 026_160_00285410 | Lymphoma | 160 | GRANTA-519 | 456 | 4.112789 | 0.631 | 0.706 | 0.761 | 0.7798 | 0.7991 | 0.771 | 0.9167 | 0.9366 | 0.9971 |
| 026_144_00287710 | Lymphoma | 1444 | BL-41 | 456 | 4.441898 | 0.536 | 1.076 | 0.941 | 0.9045 | 0.8922 | 0.893 | 0.9542 | 0.9696 | 1.0289 |
| 026_184_00290800 | Lymphoma | 184 | L-428 | 456 | 4.489131 | 0.614 | 0.705 | 0.723 | 0.7315 | 0.7351 | 0.721 | 1.0011 | 0.7501 | 0.8687 |
| 026_128_00290690 | Lymphoma | 128 | Farage | 456 | 4.585156 | 0.766 | 0.834 | 0.861 | 0.8151 | 0.9324 | 0.908 | 1.3723 | 1.1697 | 1.2229 |
| 026_250_00285660 | Lymphoma | 250 | WSU-NHL | 456 | 4.693667 | 0.719 | 0.765 | 0.866 | 0.8242 | 0.9178 | 0.947 | 0.9725 | 0.9585 | 0.9714 |
| 026_182_00290700 | Lymphoma | 182 | L-1236 | 456 | 4.714251 | 0.76 | 0.859 | 0.875 | 0.8522 | 0.9702 | 0.962 | 1.0483 | 0.9432 | 1.3018 |
| 026_95_00283510 | Lymphoma | 95 | HT | 456 | 4.721481 | 0.719 | 0.842 | 0.88 | 0.8266 | 0.8534 | 0.902 | 1.0348 | 0.9564 | 0.966 |
| 026_264_00288840 | Lymphoma | 264 | TK | 456 | 4.750226 | 0.794 | 0.966 | 0.936 | 0.9709 | 1.0212 | 1.024 | 1.0348 | 1.0318 | 1.0681 |
| 026_266_00287760 | Lymphoma | 266 | SLVL | 456 | 4.90866 | 0.731 | 0.786 | 0.794 | 0.8012 | 0.8742 | 0.873 | 0.9435 | 0.9199 | 0.977 |
| 026_75_00282830 | Lymphoma | 75 | Jiyoye | 456 | 4.937844 | 0.801 | 0.99 | 0.974 | 1.0523 | 1.196 | 1.107 | 1.0641 | 1.1044 | 1.0749 |
| 026_111_00291360 | Lymphoma | 111 | Hs 445 | 456 | 5.022248 | 0.704 | 0.734 | 0.703 | 0.794 | 0.7451 | 0.779 | 0.8243 | 0.8675 | 0.946 |
| 026_8151_00288540 | Lymphoma | 8151 | Ramos- | 456 | 5.147746 | 0.242 | 0.868 | 0.954 | 0.9827 | 1.022 | 0.974 | 0.9967 | 0.9674 | 1.0529 |
| | | | 2G6-4C10 | | | | | | | | | | | |
| 026_156_00287720 | Lymphoma | 156 | DG-75 | 456 | 5.222394 | 0.834 | 1.001 | 1.03 | 1.0349 | 0.9848 | 0.996 | 1.012 | 0.9965 | 0.9857 |
| 026_243_00296620 | Lymphoma | 243 | SUP-M2 | 456 | 5.25553 | 0.648 | 0.888 | 0.96 | 0.9351 | 0.9518 | 0.968 | 0.9742 | 0.9627 | 0.9659 |
| 026_86_00287870 | Lymphoma | 86 | EB2 | 456 | 5.287284 | 0.86 | 1.017 | 1.01 | 1.0356 | 1.0217 | 1.05 | 1.0267 | 1.0118 | 1.0227 |
| 026_162_00288790 | Lymphoma | 162 | HDLM-2 | 456 | 5.425219 | 0.877 | 0.903 | 0.797 | 0.9228 | 1.0293 | 1.043 | 1.0157 | 1.0517 | 1.0837 |
| 026_172_00287740 | Lymphoma | 172 | KARPAS-299 | 456 | 5.433867 | 0.832 | 0.891 | 0.95 | 0.9494 | 0.955 | 0.954 | 0.9499 | 0.9633 | 0.9487 |
| 026_235_00285430 | Lymphoma | 235 | SU-DHL-1 | 456 | 6.401484 | 0.835 | 0.997 | 0.987 | 0.9424 | 0.9387 | 0.884 | 0.8642 | 0.9464 | 0.9439 |
| 026_143_00288690 | Lymphoma | 143 | AMO-1 | 456 | 6.692908 | 0.924 | 1.025 | 1.129 | 1.034 | 1.1112 | 1.034 | 1.0278 | 1.0136 | 1.0255 |
| 026_193_00288830 | Lymphoma | 193 | MHH-PREB-1 | 456 | 6.799634 | 0.622 | 0.958 | 1.012 | 1.0149 | 1.0368 | 1.025 | 1.0064 | 0.9803 | 0.989 |
| 026_236_00288730 | Lymphoma | 236 | SU-DHL-10 | 456 | 6.99662 | 0.175 | 0.993 | 1.05 | 1.0763 | 1.0476 | 1.029 | 1.0509 | 1.0397 | 1.0494 |
| 026_81_00288530 | Lymphoma | 81 | GA-10 | 456 | 7.031749 | 0.608 | 1.003 | 1.009 | 1.1031 | 1.0252 | 1.021 | 1.0113 | 1.0077 | 1.0848 |
| 026_251_00287840 | Lymphoma | 251 | YT | 456 | 7.411746 | 0.64 | 0.986 | 1.008 | 1.0201 | 1.0204 | 1.022 | 1.0267 | 0.9848 | 1.0079 |
| 026_178_00288810 | Lymphoma | 178 | KM-H2 | 456 | 7.739504 | 1.008 | 1.009 | 0.976 | 1.0197 | 1.0404 | 1.036 | 1.0412 | 1.0156 | 1.0276 |
| 026_249_00285650 | Lymphoma | 249 | WSU-DLCL2 | 456 | 8.212885 | 1.047 | 1.226 | 1.172 | 1.1142 | 1.0869 | 1.071 | 0.9837 | 1.0247 | 1.0207 |
| 026_238_00288740 | Lymphoma | 238 | SU-DHL-4 | 456 | 8.647656 | 1.061 | 1.077 | 1.045 | 1.0257 | 1.0384 | 1.029 | 1.0236 | 1.018 | 1.0204 |
| 026_131_00287930 | Lymphoma | 131 | RL | 456 | 8.679308 | 1.124 | 1.094 | 1.048 | 1.024 | 1.0293 | 1.012 | 1.0293 | 0.9824 | 1.0061 |
| 026_915_00269070 | Miscellaneous | 915 | Hs 633T | 456 | 1.797943 | 0.344 | 0.532 | 0.607 | 0.6061 | 0.6357 | 0.736 | 0.8936 | 1.0346 | 1.1375 |
| 026_911_00269060 | Miscellaneous | 911 | GCT | 456 | 2.424125 | 0.433 | 0.617 | 0.642 | 0.6508 | 0.655 | 0.804 | 0.7763 | 0.9066 | 0.9967 |
| 026_8172_00269460 | Miscellaneous | 8172 | SW872 | 456 | 3.196748 | 0.511 | 0.716 | 0.719 | 0.7459 | 0.8264 | 0.881 | 0.9107 | 0.9313 | 1.0224 |
| 026_913_00271970 | Miscellaneous | 913 | JAR | 456 | 3.876744 | 0.685 | 0.695 | 0.794 | 0.851 | 0.9936 | 1.011 | 0.9013 | 1.0881 | 1.0491 |
| 026_8194_00271980 | Miscellaneous | 8194 | JEG-3 | 456 | 3.901223 | 0.623 | 0.808 | 0.876 | 0.9097 | 0.9037 | 0.874 | 0.9014 | 0.943 | 1.1354 |
| 026_8171_00269120 | Miscellaneous | 8171 | SW684 | 456 | 3.978538 | 0.693 | 0.91 | 0.98 | 1.1114 | 1.0275 | 1.032 | 0.9881 | 0.9218 | 1.0725 |
| 026_8112_00269450 | Miscellaneous | 8112 | MFH-ino | 456 | 4.013788 | 0.691 | 0.674 | 0.795 | 0.8068 | 0.8378 | 0.91 | 0.9824 | 1.0389 | 1.0625 |
| 026_916_00271320 | Miscellaneous | 916 | HT 1080 | 456 | 4.254215 | 0.695 | 0.804 | 0.766 | 0.7943 | 0.8276 | 0.873 | 1.1135 | 1.1006 | 1.1093 |
| 026_8004_00271280 | Miscellaneous | 8004 | A388 | 456 | 4.494172 | 0.743 | 0.793 | 0.841 | 0.8441 | 0.8899 | 0.888 | 1.1408 | 0.925 | 1.1327 |
| 026_8112_00271340 | Miscellaneous | 8112 | MFH-ino | 456 | 4.624376 | 0.721 | 0.732 | 0.741 | 0.7501 | 0.7753 | 0.799 | 1.017 | 0.9044 | 1.0866 |
| 026_8004_00269400 | Miscellaneous | 8004 | A388 | 456 | 4.778414 | 0.747 | 0.764 | 0.776 | 0.8164 | 0.8628 | 0.861 | 0.9217 | 0.8924 | 1.0697 |
| 026_8192_00269470 | Miscellaneous | 8192 | VA-ES-BJ | 456 | 4.956405 | 0.78 | 0.867 | 0.874 | 0.984 | 0.9192 | 0.889 | 0.9737 | 1.0227 | 1.0186 |
| 026_8194_00269440 | Miscellaneous | 8194 | JEG-3 | 456 | 5.058221 | 0.786 | 0.87 | 1.048 | 0.908 | 0.8876 | 0.984 | 0.8804 | 1.0294 | 1.0609 |
| 026_913_00269430 | Miscellaneous | 913 | JAR | 456 | 5.170211 | 0.816 | 0.841 | 0.849 | 0.8709 | 0.8661 | 0.88 | 1.0502 | 1.0585 | 1.0591 |
| 026_916_00269420 | Miscellaneous | 916 | HT 1080 | 456 | 5.302771 | 0.831 | 0.891 | 0.904 | 0.9053 | 0.9327 | 0.978 | 0.9465 | 1.1023 | 1.1108 |
| 026_8175_00269140 | Miscellaneous | 8175 | SW982 | 456 | 5.586364 | 0.832 | 0.968 | 0.988 | 1.1946 | 1.0638 | 1.017 | 1.0728 | 1.1006 | 1.2781 |
| 026_1225_00269660 | Muscle | 1255 | RD | 456 | 2.694207 | 0.612 | 0.595 | 0.602 | 0.575 | 0.6051 | 0.678 | 0.8404 | 0.9183 | 0.947 |
| 26_135_00271680 | Muscle | 135 | SJCRH30 | 456 | 2.740947 | 0.247 | 0.61 | 0.713 | 0.8654 | 0.8633 | 0.854 | 0.9433 | 0.9991 | 1.105 |
| 026_924_00269640 | Muscle | 924 | A673 | 456 | 2.779569 | 0.563 | 0.554 | 0.607 | 0.6884 | 0.7085 | 0.808 | 0.8122 | 0.8796 | 0.9437 |
| 26_562_00271660 | Muscle | 562 | KYM-1 | 456 | 2.818237 | 0.433 | 0.665 | 0.727 | 0.7663 | 0.7943 | 0.835 | 0.9132 | 0.9948 | 0.9596 |
| 026_135_00271410 | Muscle | 135 | SJCRH30 | 456 | 3.059385 | 0.345 | 0.672 | 0.79 | 0.8395 | 0.8741 | 0.957 | 0.9768 | 1.032 | 1.1796 |
| 026_923_00269680 | Muscle | 923 | RH-41 | 456 | 3.133389 | 0.309 | 0.65 | 0.731 | 0.7895 | 0.6971 | 0.795 | 0.7303 | 0.9458 | 0.9325 |
| 026_562_00270060 | Muscle | 562 | KYM-1 | 456 | 3.396905 | 0.586 | 0.748 | 0.78 | 0.8707 | 0.8698 | 0.955 | 1.0372 | 1.0323 | 1.0217 |
| 026_920_00269670 | Muscle | 920 | RH-1 | 456 | 3.433085 | 0.581 | 0.772 | 0.846 | 0.9032 | 0.9238 | 0.951 | 0.9496 | 0.9615 | 0.9852 |
| 026_135_00270070 | Muscle | 135 | SJCRH30 | 456 | 3.759361 | 0.326 | 0.727 | 0.825 | 0.8737 | 0.9084 | 0.931 | 0.9701 | 0.9946 | 0.9435 |
| 026_919_00269630 | Muscle | 919 | A-204 | 456 | 5.25605 | 0.778 | 0.798 | 0.868 | 0.8744 | 0.861 | 0.89 | 0.9735 | 0.9392 | 0.9765 |
| 026_921_00285151 | Muscle | 921 | RH-18 | 456 | 5.863422 | 0.84 | 0.987 | 0.961 | 0.9653 | 0.9811 | 0.903 | 0.8875 | 0.9228 | 0.9815 |
| 026_8182_00293791 | Muscle | 8182 | TE-441-T | 456 | 6.133177 | 0.848 | 1.001 | 1.103 | 1.0954 | 1.1007 | 0.855 | 0.8882 | 1.0698 | 0.8069 |
| 026_369_00258500 | Nervous | 369 | CHP-212 | 456 | −4.151196 | 0.165 | 0.184 | 0.183 | 0.1873 | 0.1985 | 0.219 | 0.2948 | 0.3285 | 0.4946 |
| | System | | | | | | | | | | | | | |
| 026_390_00262920 | Nervous | 390 | NB69 | 456 | −1.3827 | 0.056 | 0.074 | 0.068 | 0.0948 | 0.096 | 0.163 | 0.4343 | 0.6938 | 0.8833 |
| | System | | | | | | | | | | | | | |
| 026_629_00316570 | Nervous | 629 | NB(TU)1-10 | 456 | −0.654294 | 0.322 | 0.361 | 0.388 | 0.398 | 0.4347 | 0.594 | 0.7533 | 0.6321 | 0.6782 |
| | System | | | | | | | | | | | | | |
| 026_366_00257090 | Nervous | 366 | BE(2)-M17 | 456 | −0.258429 | 0.204 | 0.352 | 0.333 | 0.3872 | 0.4128 | 0.547 | 0.6415 | 0.7687 | 0.9298 |
| | System | | | | | | | | | | | | | |
| 026_384_00314240 | Nervous | 384 | MHH-NB-11 | 456 | 1.316182 | 0.368 | 0.42 | 0.48 | 0.6318 | 0.5903 | 0.783 | 0.7802 | 0.9243 | 0.5317 |
| | System | | | | | | | | | | | | | |
| 026_385_00314290 | Nervous | 385 | SIMA | 456 | 1.359572 | 0.402 | 0.496 | 0.488 | 0.5135 | 0.6006 | 0.629 | 0.8787 | 0.8808 | 1.0864 |
| | System | | | | | | | | | | | | | |
| 026_630_00264810 | Nervous | 630 | NH-12 | 456 | 1.516245 | 0.143 | 0.443 | 0.537 | 0.5763 | 0.6816 | 0.684 | 0.7263 | 0.9504 | 0.791 |
| | System | | | | | | | | | | | | | |
| 026_8124_00308720 | Nervous | 8124 | NB14 | 456 | 1.784337 | 0.802 | 0.401 | 0.6 | 0.6379 | 0.6871 | 0.943 | 0.8827 | 0.8247 | 0.9343 |
| | System | | | | | | | | | | | | | |
| 026_639_00271120 | Nervous | 639 | GOTO | 456 | 1.784838 | 0.174 | 0.52 | 0.54 | 0.6353 | 0.6485 | 0.726 | 0.9341 | 0.8908 | 1.1295 |
| | System | | | | | | | | | | | | | |
| 026_8094_00314230 | Nervous | 8094 | LAN-6 | 456 | 1.824343 | 0.55 | 0.565 | 0.476 | 0.5734 | 0.6862 | 0.656 | 0.7893 | 0.9264 | 0.7016 |
| | System | | | | | | | | | | | | | |
| 026_8076_00260401 | Nervous | 8076 | IMR-5 | 456 | 1.864038 | 0.346 | 0.489 | 0.575 | 1.2246 | 0.8619 | 0.823 | 1.0781 | 0.7607 | 0.8894 |
| | System | | | | | | | | | | | | | |
| 026_8127_00258950 | Nervous | 8127 | NB5 | 456 | 2.106125 | 0.202 | 0.628 | 0.611 | 0.6823 | 0.725 | 0.845 | 0.9541 | 0.9703 | 1.0012 |
| | System | | | | | | | | | | | | | |
| 026_8124_00311130 | Nervous | 8124 | NB14 | 456 | 2.216808 | 0.544 | 0.563 | 0.516 | 0.5711 | 0.5857 | 0.776 | 0.9014 | 0.9091 | 1.0395 |
| | System | | | | | | | | | | | | | |
| 026_8126_00264800 | Nervous | 8126 | NB17 | 456 | 2.289546 | 0.347 | 0.54 | 0.684 | 0.7567 | 0.8851 | 0.929 | 0.8729 | 0.9752 | 0.9196 |
| | System | | | | | | | | | | | | | |
| 026_641_00308180 | Nervous | 641 | TGW | 456 | 2.326645 | 0.4556 | 0.502 | 0.593 | 0.7234 | 0.8006 | 0.88 | 0.9296 | 0.9651 | 0.9821 |
| | System | | | | | | | | | | | | | |
| 026_8121_00256290 | Nervous | 8121 | NB10 | 456 | 2.356578 | 0.646 | 0.568 | 0.562 | 0.5835 | 0.6642 | 0.613 | 0.5487 | 0.6035 | 1.0005 |
| | System | | | | | | | | | | | | | |
| 026_8220_00308810 | Nervous | 8220 | KP-N-YN | 456 | 2.470465 | 0.366 | 0.619 | 0.752 | 0.7815 | 0.7642 | 0.87 | 0.8457 | 0.8335 | 0.8942 |
| | System | | | | | | | | | | | | | |
| 026_8090_00263920 | Nervous | 8090 | KP-N-YS | 456 | 2.547617 | 0.267 | 0.583 | 0.69 | 0.9135 | 0.905 | 0.924 | 0.9505 | 0.9232 | 1.012 |
| | System | | | | | | | | | | | | | |
| 026_363_00252730 | Nervous | 363 | SK-N-FI | 456 | 2.667265 | 0.519 | 0.583 | 0.574 | 0.6332 | 0.5059 | 0.571 | 0.7766 | 0.6898 | 0.9188 |
| | System | | | | | | | | | | | | | |
| 026_382_00311110 | Nervous | 382 | KELLY | 456 | 3.39659 | 0.518 | 0.757 | 0.767 | 0.7123 | 0.7538 | 0.883 | 0.8718 | 0.8733 | 0.9755 |
| | System | | | | | | | | | | | | | |
| 026_8064_00259120 | Nervous | 8064 | GI-ME-N | 456 | 3.426922 | 0.582 | 0.763 | 0.78 | 0.8295 | 0.8371 | 0.955 | 1.0539 | 1.0652 | 1.104 |
| | System | | | | | | | | | | | | | |
| 026_8195_00318640 | Nervous | 8195 | CHP-134 | 456 | 3.519288 | 0.6 | 0.8 | 0.86 | 0.909 | 0.9931 | 0.919 | 0.9533 | 0.941 | 1.0273 |
| | System | | | | | | | | | | | | | |
| 026_382_00308690 | Nervous | 382 | KELLY | 456 | 3.678148 | 0.59 | 0.706 | 0.666 | 0.7413 | 0.8277 | 0.814 | 0.8335 | 0.8742 | 0.9843 |
| | System | | | | | | | | | | | | | |
| 026_8129_00314250 | Nervous | 8129 | NB7 | 456 | 3.824042 | 0.644 | 0.861 | 0.945 | 0.972 | 0.9655 | 0.998 | 1.0941 | 1.0771 | 1.0471 |
| | System | | | | | | | | | | | | | |
| 026_8126_00280241 | Nervous | 8126 | NB17 | 456 | 3.845261 | 0.611 | 0.771 | 0.746 | 1.0569 | 0.8567 | 0.864 | 0.9408 | 0.9041 | 0.972 |
| | System | | | | | | | | | | | | | |
| 026_8007_00252850 | Nervous | 8007 | ACN | 456 | 3.989797 | 0.411 | 0.72 | 0.821 | 0.7359 | 0.7717 | 0.797 | 0.8528 | 1.0702 | 0.9564 |
| | System | | | | | | | | | | | | | |
| 026_8122_00273460 | Nervous | 8122 | NB12 | 456 | 4.358809 | 0.15 | 0.809 | 0.911 | 0.9912 | 0.9113 | 1.021 | 0.9055 | 0.8723 | 1.0083 |
| | System | | | | | | | | | | | | | |
| 026_8226_00252530 | Nervous | 8226 | NBsus SR | 456 | 4.365592 | 1.14 | 0.81 | 0.878 | 0.6567 | 0.6737 | 0.94 | 0.7615 | 0.8399 | 1.1136 |
| | System | | | | | | | | | | | | | |
| 026_8064_00256670 | Nervous | 8064 | GI-ME-N | 456 | 4.809816 | 0.398 | 0.871 | 0.839 | 0.9402 | 0.8757 | 0.96 | 0.9284 | 0.9838 | 1.0451 |
| | System | | | | | | | | | | | | | |
| 026_8128_00257200 | Nervous | 8128 | NB6 | 456 | 4.886145 | 0.836 | 0.93 | 0.956 | 1.0121 | 0.9926 | 1.008 | 0.9893 | 0.9944 | 0.9472 |
| | System | | | | | | | | | | | | | |
| 026_8123_00256300 | Nervous | 8123 | NB13 | 456 | 5.775254 | 0.837 | 1.028 | 1.034 | 0.9756 | 1.0272 | 0.974 | 0.9893 | 0.9781 | 0.9486 |
| | System | | | | | | | | | | | | | |
| 026_396_00261020 | Nervous | 396 | NB-1 | 456 | 5.873385 | 0.813 | 1.039 | 0.892 | 0.8527 | 0.9085 | 0.934 | 0.9583 | 0.9709 | 1.0441 |
| | System | | | | | | | | | | | | | |
| 026_370_00252740 | Nervous | 370 | SK-N-SH | 456 | 6.176056 | 0.926 | 0.811 | 0.891 | 1.0803 | 2.0427 | 1.01 | 0.892 | 0.9269 | 0.9864 |
| | System | | | | | | | | | | | | | |
| 026_364_00252720 | Nervous | 364 | SK-N-DZ | 456 | 6.251974 | 0.895 | 1.012 | 0.958 | 1.0963 | 0.9642 | 0.976 | 1.0005 | 0.9787 | 0.9774 |
| | System | | | | | | | | | | | | | |
| 026_370_00258530 | Nervous | 370 | SK-N-SH | 456 | 6.266144 | 0.36 | 0.933 | 1.052 | 1.0961 | 1.0754 | 0.948 | 0.9798 | 1.0134 | 0.9668 |
| | System | | | | | | | | | | | | | |
| 026_362_00252710 | Nervous | 362 | SK-N-AS | 456 | 6.874316 | 0.911 | 1.075 | 0.916 | 1.0354 | 0.9179 | 0.939 | 1.178 | 0.9451 | 0.9362 |
| | System | | | | | | | | | | | | | |
| 026_368_00252700 | Nervous | 368 | MC-IXC | 456 | 6.982443 | 0.084 | 0.616 | 1.087 | 1.1559 | 1.0678 | 1.11 | 1.0784 | 0.9917 | 0.9902 |
| | System | | | | | | | | | | | | | |
| 026_8122_00262490 | Nervous | 8122 | NB12 | 456 | 7.149853 | 0.063 | 0.973 | 1.019 | 1.3067 | 1.0528 | 0.999 | 1.4063 | 0.9955 | 0.9632 |
| | System | | | | | | | | | | | | | |
| 026_934_00287430 | Ovary | 934 | A2780 | 456 | −0.227264 | 0.302 | 0.3 | 0.342 | 0.3503 | 0.3758 | 0.467 | 0.7239 | 0.7489 | 0.9268 |
| 026_1126_00293760 | Ovary | 1126 | OV-90 | 456 | 0.856076 | 0.323 | 0.361 | 0.384 | 0.4703 | 0.5785 | 0.701 | 0.8275 | 0.885 | 0.873 |
| 026_1129_00290660 | Ovary | 1129 | TOV-112D | 456 | 2.270257 | 0.47 | 0.481 | 0.592 | 0.7498 | 0.7692 | 0.925 | 0.9217 | 0.9942 | 1.1258 |
| 026_1220_00291150 | Ovary | 1220 | ES-2 | 456 | 2.397868 | 0.584 | 0.504 | 0.584 | 0.5655 | 0.9851 | 0.957 | 1.1602 | 1.1706 | 1.0966 |
| 026_949_00290350 | Ovary | 949 | TYK-nu | 456 | 2.489488 | 0.511 | 0.577 | 0.587 | 0.5768 | 0.6153 | 0.686 | 0.7718 | 1.0687 | 1.0378 |
| 026_925_00290850 | Ovary | 925 | IGROV-1 | 456 | 2.753994 | 0.494 | 0.608 | 0.578 | 0.7594 | 0.7462 | 0.857 | 0.9169 | 0.8966 | 0.8943 |
| 026_8244_00314220 | Ovary | 8244 | JHOS-4 | 456 | 2.779507 | 0.63 | 0.601 | 0.597 | 0.6381 | 0.7711 | 0.88 | 0.955 | 0.9128 | 0.9028 |
| 026_8279_00293400 | Ovary | 8279 | UWB1.289 | 456 | 2.893774 | 0.764 | 0.625 | 0.723 | 0.7182 | 0.783 | 1.079 | 0.9597 | 1.044 | 1.0467 |
| 026_940_00290340 | Ovary | 940 | RMG-I | 456 | 3.11591 | 0.587 | 0.604 | 0.66 | 0.6995 | 0.6871 | 0.846 | 0.8531 | 0.9424 | 1.0257 |
| 26_8238_00304370 | Ovary | 8238 | IOSE-364- | 456 | 3.149354 | 0.505 | 0.763 | 0.761 | 0.7451 | 0.8102 | 0.848 | 1.0558 | 1.0226 | 1.0295 |
| 026_8237_00295840 | Ovary | 8237 | Hey | 456 | 3.317969 | 0.551 | 0.783 | 0.688 | 0.776 | 0.8484 | 0.917 | 1.1038 | 1.0681 | 1.1892 |
| 026_8260_00292720 | Ovary | 8260 | PEO1 | 456 | 3.508406 | 0.648 | 0.61 | 0.642 | 0.7269 | 0.6891 | 0.74 | 1.0027 | 0.9408 | 0.9148 |
| 026_8230_00303100 | Ovary | 8230 | DOV13 | 456 | 3.68994 | 0.636 | 0.73 | 0.886 | 0.851 | 0.9406 | 0.961 | 0.9388 | 0.9681 | 1.0998 |
| 026_8230_00292650 | Ovary | 8230 | DOV13 | 456 | 3.809459 | 0.641 | 0.685 | 0.768 | 0.9488 | 0.8764 | 0.863 | 0.9076 | 0.9226 | 1.0565 |
| 026_8092_00295850 | Ovary | 8092 | KURAMOCHI | 456 | 3.819916 | 0.757 | 0.729 | 0.642 | 0.6589 | 0.7541 | 0.914 | 0.9211 | 1.0035 | 1.0531 |
| 026_8240_00291160 | Ovary | 8240 | IOSE-523- | 456 | 3.834885 | 0.676 | 0.747 | 0.827 | 0.9249 | 0.9868 | 1 | 0.9181 | 0.9742 | 1.0197 |
| 026_932_00291140 | Ovary | 932 | EFO-27 | 456 | 3.943589 | 0.65 | 0.668 | 0.65 | 0.7216 | 0.7753 | 0.887 | 0.9104 | 0.9136 | 0.9152 |
| 026_8084_00292670 | Ovary | 8084 | KGN | 456 | 4.00968 | 0.776 | 0.686 | 0.68 | 0.804 | 0.816 | 0.93 | 0.911 | 0.9826 | 0.9092 |
| 026_1125_00290571 | Ovary | 1125 | Caov-3 | 456 | 4.050855 | 0.676 | 0.787 | 0.855 | 0.951 | 0.9041 | 1.029 | 0.9092 | 0.8992 | 1.0107 |
| 026_8256_00292920 | Ovary | 8256 | OV-7 | 456 | 4.064163 | 0.666 | 0.844 | 0.821 | 0.9099 | 0.9293 | 0.961 | 1.0098 | 0.9638 | 0.9379 |
| 026_8241_00292900 | Ovary | 8241 | IOSE-75-16SV40 | 456 | 4.094216 | 0.698 | 0.804 | 0.834 | 0.8672 | 0.894 | 1.02 | 1.0443 | 1.0711 | 1.107 |
| 026_938_00287450 | Ovary | 938 | OAW42 | 456 | 4.158942 | 0.689 | 0.707 | 0.728 | 0.8613 | 0.7453 | 0.912 | 0.7815 | 1.0258 | 1.0515 |
| 026_1235_00298501 | Ovary | 1235 | Caov-4 | 456 | 4.19878 | 0.673 | 0.815 | 0.851 | 0.7794 | 0.8482 | 0.895 | 1.0046 | 1.0048 | 0.9449 |
| 026_1221_00293780 | Ovary | 1221 | SW 626 | 456 | 4.261643 | 0.636 | 0.704 | 0.69 | 0.6894 | 0.7014 | 0.755 | 0.8172 | 0.8859 | 1.0281 |
| 026_933_00295510 | Ovary | 933 | FU-OV-1 | 456 | 4.35795 | 0.921 | 0.786 | 0.66 | 0.785 | 0.7855 | 0.932 | 1.0797 | 1.0643 | 1.0563 |
| 026_8259_00292710 | Ovary | 8259 | OVK-18 | 456 | 4.419332 | 0.714 | 0.849 | 0.926 | 0.7816 | 0.8328 | 1.11 | 1.0158 | 0.9241 | 1.1619 |
| 026_8242_00295400 | Ovary | 8242 | JHOS-2 | 456 | 4.46038 | 0.744 | 0.845 | 0.843 | 0.8069 | 0.9687 | 1.09 | 0.9491 | 1.0683 | 1.0333 |
| 026_1128_00292620 | Ovary | 1128 | PA-1 | 456 | 4.524728 | 0.741 | 0.875 | 0.809 | 0.8925 | 0.9409 | 1.005 | 1.0033 | 1.0111 | 1.0682 |
| 026_938_00290330 | Ovary | 938 | OAW42 | 456 | 4.600882 | 0.772 | 0.772 | 0.843 | 0.9103 | 0.8456 | 1.001 | 1.0305 | 1.0314 | 1.0721 |
| 026_8148_00292700 | Ovary | 8148 | OVCAR-4 | 456 | 4.649732 | 0.715 | 0.824 | 0.723 | 0.6704 | 0.8391 | 0.855 | 0.9034 | 1.0922 | 1.0169 |
| 026_8243_00295410 | Ovary | 8243 | JHOS-3 | 456 | 4.699022 | 0.801 | 0.758 | 0.691 | 0.7427 | 0.8184 | 0.86 | 1.0104 | 0.9653 | 1.0478 |
| 026_1130_00308501 | Ovary | 1130 | TOV-21G | 456 | 4.743414 | 0.744 | 0.838 | 0.882 | 0.8952 | 0.9369 | 0.922 | 0.9212 | 0.9456 | 0.9683 |
| 026_931_00252940 | Ovary | 931 | EFO-21 | 456 | 4.786052 | 0.749 | 0.962 | 0.957 | 1.0202 | 1.0143 | 0.983 | 0.9976 | 0.9688 | 0.9523 |
| 026_931_00290820 | Ovary | 931 | EFO-21 | 456 | 4.852802 | 0.791 | 0.924 | 0.954 | 0.9827 | 1.0247 | 0.961 | 0.9353 | 0.9274 | 0.9237 |
| 026_932_00288450 | Ovary | 932 | EFO-27 | 456 | 4.890908 | 0.759 | 0.729 | 0.783 | 0.7813 | 0.828 | 0.851 | 0.9362 | 1.0032 | 1.0515 |
| 026_8258_00291180 | Ovary | 8258 | OVCA433 | 456 | 4.901177 | 0.703 | 0.713 | 0.753 | 0.7366 | 0.79 | 0.864 | 0.9468 | 0.9244 | 0.9669 |
| 026_928_00290870 | Ovary | 928 | OVCAR-5 | 456 | 4.903025 | 0.791 | 0.718 | 0.699 | 0.6913 | 0.7147 | 0.8 | 0.831 | 0.8925 | 0.9149 |
| 026_8257_00293650 | Ovary | 8257 | OVCA420 | 456 | 4.98883 | 0.716 | 0.738 | 0.79 | 0.8055 | 0.8238 | 0.819 | 0.8677 | 0.8812 | 1.0222 |
| 026_8257_00292690 | Ovary | 8257 | OVCA420 | 456 | 5.050268 | 0.789 | 0.822 | 0.798 | 0.9932 | 0.8382 | 0.876 | 0.928 | 0.9213 | 1.1024 |
| 026_8239_00292890 | Ovary | 8239 | IOSE-397 | 456 | 5.092878 | 0.842 | 0.8 | 0.866 | 0.8796 | 0.8926 | 0.921 | 1.2015 | 0.9976 | 1.2125 |
| 026_941_00288490 | Ovary | 941 | RKN | 456 | 5.095259 | 0.777 | 0.883 | 0.798 | 0.796 | 0.8682 | 1.049 | 1.195 | 1.0697 | 1.0119 |
| 026_929_00290880 | Ovary | 929 | OVCAR-8 | 456 | 5.147098 | 0.763 | 0.805 | 0.822 | 0.8457 | 0.8675 | 0.888 | 0.9211 | 0.9687 | 1.0047 |
| 026_8215_00287660 | Ovary | 8215 | OC-314 | 456 | 5.38803 | 0.869 | 0.99 | 0.986 | 1.0451 | 1.0092 | 1.01 | 1.0495 | 0.9963 | 1.0021 |
| 026_939_00287460 | Ovary | 939 | SK-OV-3 | 456 | 5.430154 | 0.806 | 0.848 | 0.834 | 0.8094 | 0.8719 | 0.891 | 0.9424 | 0.903 | 1.1026 |
| 026_945_00288480 | Ovary | 945 | OVMIU | 456 | 5.797932 | 0.75 | 0.737 | 0.748 | 0.7949 | 0.7921 | 0.844 | 0.8857 | 0.9368 | 0.9866 |
| 026_948_00291230 | Ovary | 948 | OVTOKO | 456 | 5.89738 | 0.829 | 0.912 | 0.942 | 0.9105 | 0.938 | 0.909 | 0.9025 | 0.9329 | 0.9575 |
| 026_8255_00293640 | Ovary | 8255 | OV-56 | 456 | 5.989644 | 0.874 | 0.894 | 0.904 | 0.9159 | 0.9189 | 0.911 | 0.9267 | 0.9453 | 0.9277 |
| 026_937_00288460 | Ovary | 937 | OAW28 | 456 | 6.027686 | 0.859 | 0.878 | 0.802 | 0.8512 | 0.7724 | 0.814 | 1.0447 | 0.8649 | 1.0363 |
| 026_1127_00296460 | Ovary | 1127 | NIH1OVCAR-3 | 456 | 6.109694 | 0.886 | 0.95 | 0.992 | 0.9785 | 0.8788 | 0.986 | 0.9619 | 1.0732 | 0.8745 |
| 026_947_00263500 | Ovary | 947 | OVKATE | 456 | 6.266319 | 0.819 | 0.823 | 0.875 | 0.8874 | 0.9093 | 0.906 | 0.9175 | 0.9563 | 0.9507 |
| 026_938_00292910 | Ovary | 938 | OAW42 | 456 | 6.299032 | 0.918 | 0.963 | 0.958 | 0.9614 | 0.9778 | 1.041 | 1.0213 | 0.9899 | 1.0087 |
| 026_945_00256180 | Ovary | 945 | OVMIU | 456 | 6.360298 | 0.891 | 0.987 | 1.001 | 0.9225 | 0.8452 | 0.776 | 0.8273 | 0.9233 | 1.0102 |
| 026_946_00291190 | Ovary | 946 | OVISE | 456 | 6.600815 | 0.805 | 0.739 | 0.737 | 0.7358 | 0.7593 | 0.759 | 0.8539 | 0.8664 | 0.8624 |
| 026_8254_00295890 | Ovary | 8254 | OV-17R | 456 | 6.877329 | 0.913 | 1.041 | 0.974 | 1.0282 | 1.0077 | 1.015 | 1.0376 | 0.9484 | 1.0604 |
| 026_973_00295540 | Pancreas | 973 | HUP-T4 | 456 | 0.866739 | 0.321 | 0.315 | 0.362 | 0.4721 | 0.5603 | 0.814 | 1.0984 | 0.8938 | 1.1407 |
| 026_983_00295450 | Pancreas | 983 | SUIT-2 | 456 | 1.197889 | 0.397 | 0.339 | 0.382 | 0.581 | 0.6565 | 0.942 | 0.9937 | 1.019 | 1.1926 |
| 026_982_00292940 | Pancreas | 982 | QGP-1 | 456 | 1.430625 | 0.538 | 0.437 | 0.492 | 0.5682 | 0.6307 | 0.8 | 0.8372 | 0.9601 | 1.0476 |
| 026_8118_00295430 | Pancreas | 8118 | MZ1-PC | 456 | 1.48113 | 0.363 | 0.489 | 0.495 | 0.5335 | 0.6471 | 0.671 | 0.9118 | 1.008 | 0.9838 |
| 026_8149_00293771 | Pancreas | 8149 | PSN1 | 456 | 1.829333 | 0.531 | 0.553 | 0.5 | 0.5231 | 0.6104 | 0.71 | 0.8375 | 0.909 | 0.9509 |
| 026_953_00295470 | Pancreas | 953 | AsPC-1 | 456 | 1.847893 | 0.476 | 0.515 | 0.508 | 0.4987 | 0.5741 | 0.746 | 0.8281 | 1.0377 | 1.0843 |
| 026_1256_00260300 | Pancreas | 1256 | 950-MP5 | 456 | 1.902786 | 0.332 | 0.473 | 0.515 | 0.7193 | 0.8035 | 0.916 | 0.9125 | 1.0441 | 0.9009 |
| 026_976_00298480 | Pancreas | 976 | Panc04.03 | 456 | 2.685969 | 0.592 | 0.594 | 0.57 | 0.562 | 0.6645 | 0.706 | 0.8319 | 0.9296 | 1.0176 |
| 026_954_00292870 | Pancreas | 954 | BxPC-3 | 456 | 2.90077 | 0.574 | 0.607 | 0.558 | 0.6572 | 0.7404 | 0.813 | 1.0541 | 1.0916 | 0.9092 |
| 026_967_00292570 | Pancreas | 967 | Capan-1 | 456 | 3.140577 | 0.522 | 0.69 | 0.758 | 0.8527 | 0.6713 | 0.874 | 0.9506 | 0.894 | 0.958 |
| 026_975_00295591 | Pancreas | 975 | YAPC | 456 | 3.507979 | 0.613 | 0.705 | 0.797 | 0.9196 | 0.8833 | 0.921 | 1.0616 | 1.0323 | 1.0675 |
| 026_1135_00292930 | Pancreas | 1135 | PL18 | 456 | 3.513426 | 0.636 | 0.623 | 0.694 | 0.6978 | 0.7917 | 0.832 | 0.9096 | 0.9334 | 0.9721 |
| 026_977_00308210 | Pancreas | 977 | KP-1N | 456 | 3.657778 | 0.607 | 0.764 | 0.684 | 0.7525 | 0.7655 | 0.908 | 0.9944 | 0.9837 | 0.9553 |
| 026_969_00295580 | Pancreas | 969 | PA-TU-8988T | 456 | 3.657963 | 0.604 | 0.653 | 0.725 | 0.7145 | 0.7879 | 0.992 | 1.0299 | 1.0048 | 1.0863 |
| 026_1491_00273490 | Pancreas | 1491 | SNU-324 | 456 | 3.732679 | 0.387 | 0.683 | 0.791 | 0.7879 | 0.8486 | 0.832 | 0.9455 | 0.9813 | 0.9695 |
| 026_961_00295570 | Pancreas | 961 | Panc02.03 | 456 | 4.088522 | 0.702 | 0.699 | 0.633 | 0.701 | 0.7003 | 0.795 | 0.8748 | 0.9019 | 1.1113 |
| 026_974_00292601 | Pancreas | 974 | HUP-T3 | 456 | 4.280085 | 0.684 | 0.754 | 0.793 | 0.909 | 0.7596 | 0.88 | 0.9895 | 0.9696 | 1.0099 |
| 026_963_00293710 | Pancreas | 963 | Hs 766T | 456 | 4.34359 | 0.709 | 0.746 | 0.833 | 0.7623 | 0.8169 | 1.052 | 0.9605 | 0.9551 | 1.0544 |
| 026_959_00292630 | Pancreas | 959 | Panc03.27 | 456 | 4.482408 | 0.701 | 0.682 | 0.805 | 0.8272 | 0.7565 | 0.838 | 0.9087 | 0.9434 | 1.0258 |
| 026_981_00293750 | Pancreas | 981 | KP-4 | 456 | 4.755774 | 0.733 | 0.9 | 0.784 | 0.9218 | 0.8875 | 1.007 | 0.9325 | 1.0003 | 0.8788 |
| 026_968_00292580 | Pancreas | 968 | CFPAC-1 | 456 | 4.814885 | 0.772 | 0.822 | 0.87 | 0.9195 | 0.9082 | 0.935 | 0.8889 | 1.0106 | 1.1286 |
| 026_1134_00300280 | Pancreas | 1134 | PL4 | 456 | 4.852316 | 0.738 | 0.739 | 0.727 | 0.7443 | 0.7816 | 0.797 | 0.9521 | 0.9663 | 1.0376 |
| 026_979_00293740 | Pancreas | 979 | KP-3 | 456 | 4.900084 | 0.956 | 0.777 | 0.839 | 0.8825 | 0.8836 | 0.909 | 0.9593 | 0.9092 | 0.9686 |
| 026_956_00292591 | Pancreas | 956 | HPAF-II | 456 | 5.005523 | 0.734 | 0.786 | 0.762 | 0.6523 | 0.7269 | 0.755 | 0.9185 | 0.979 | 1.0195 |
| 026_953_00257150 | Pancreas | 953 | AsPC-1 | 456 | 5.141432 | 0.761 | 0.708 | 0.731 | 0.7295 | 0.7369 | 0.79 | 0.8534 | 0.8911 | 0.9577 |
| 26_968_00304350 | Pancreas | 968 | CFPAC-1 | 456 | 5.151366 | 0.817 | 0.828 | 0.864 | 0.8724 | 0.8715 | 1.008 | 1.007 | 0.9779 | 1.001 |
| 026_957_00296350 | Pancreas | 957 | SW 1990 | 456 | 5.575766 | 0.849 | 0.911 | 0.906 | 0.8954 | 0.9494 | 0.957 | 0.977 | 0.9769 | 1.1173 |
| 026_960_00296311 | Pancreas | 960 | Panc08.13 | 456 | 5.65459 | 0.875 | 0.894 | 0.924 | 0.9669 | 0.9243 | 0.96 | 1.1005 | 1.065 | 1.0309 |
| 026_951_00256230 | Pancreas | 951 | HPAC | 456 | 5.736336 | 0.862 | 1.024 | 0.931 | 0.8532 | 0.9386 | 1.035 | 1.0328 | 1.0507 | 1.0612 |
| 026_964_00295480 | Pancreas | 964 | Capan-2 | 456 | 5.74256 | 0.877 | 0.928 | 0.759 | 0.8346 | 0.9905 | 0.925 | 1.0364 | 1.027 | 0.9499 |
| 026_1134_00298560 | Pancreas | 1134 | PL4 | 456 | 5.771259 | 0.844 | 0.812 | 0.91 | 0.8479 | 0.7738 | 0.977 | 0.9068 | 1.059 | 0.9774 |
| 026_970_00293370 | Pancreas | 970 | PA-TU-8902 | 456 | 5.772043 | 0.841 | 0.97 | 0.919 | 0.8896 | 0.9177 | 0.966 | 0.9181 | 1.322 | 0.9871 |
| 026_952_00292610 | Pancreas | 952 | MIA PaCa-2 | 456 | 5.946631 | 0.923 | 0.885 | 0.718 | 0.8919 | 0.8505 | 1.058 | 1.0586 | 0.8995 | 1.1403 |
| 026_972_00296250 | Pancreas | 972 | DAN-G | 456 | 5.955955 | 0.897 | 0.924 | 0.945 | 0.9646 | 0.9703 | 0.978 | 1.0074 | 1.032 | 1.0585 |
| 026_951_00295520 | Pancreas | 951 | HPAC | 456 | 5.962773 | 0.923 | 0.889 | 0.787 | 0.9719 | 0.9562 | 1.118 | 0.986 | 0.9708 | 1.1145 |
| 026_975_00252910 | Pancreas | 975 | YAPC | 456 | 5.989883 | 1.015 | 0.914 | 0.897 | 0.8713 | 0.8903 | 1.037 | 0.977 | 1.1409 | 0.981 |
| 026_963_00252950 | Pancreas | 963 | Hs 766T | 456 | 6.264463 | 0.496 | 0.974 | 0.953 | 1.0445 | 0.9118 | 1.028 | 0.9324 | 1.0245 | 1.0186 |
| 026_958_00296320 | Pancreas | 958 | Panc10.05 | 456 | 6.318194 | 0.851 | 0.835 | 0.851 | 0.824 | 0.8929 | 0.879 | 0.9003 | 1.0065 | 1.0291 |
| 026_955_00292640 | Pancreas | 955 | SU.86.86 | 456 | 6.593699 | 1.017 | 0.82 | 0.986 | 0.9378 | 1.1258 | 1.072 | 1.0873 | 1.0381 | 0.9789 |
| 026_759_00300240 | Pleura | 759 | MSTO-211H | 456 | 3.066599 | 0.666 | 0.637 | 0.664 | 0.6954 | 0.8026 | 0.901 | 1.0485 | 1.0165 | 1.0436 |
| 026_1213_00303050 | Pleura | 1213 | H2818 | 456 | 3.157415 | 0.577 | 0.674 | 0.728 | 0.8558 | 0.9032 | 0.962 | 0.9285 | 1.0744 | 1.1008 |
| 026_8116_00303080 | Pleura | 8116 | MPP-89 | 456 | 3.723631 | 0.416 | 0.808 | 0.823 | 0.9733 | 0.9887 | 0.968 | 1.0012 | 0.9912 | 1.0294 |
| 026_1206_00302600 | Pleura | 1206 | H2722 | 456 | 3.764667 | 0.604 | 0.768 | 0.801 | 0.8049 | 0.8692 | 0.937 | 0.9073 | 0.9842 | 0.9698 |
| 026_1210_00302610 | Pleura | 1210 | H2803 | 456 | 4.117283 | 0.686 | 0.723 | 0.669 | 0.7081 | 0.719 | 0.892 | 0.9533 | 1.0129 | 0.9547 |
| 026_1215_00311260 | Pleura | 1215 | H290 | 456 | 4.122283 | 0.629 | 0.753 | 0.69 | 0.7652 | 0.7781 | 0.737 | 0.8199 | 0.9853 | 0.9812 |
| 026_1206_00308200 | Pleura | 1206 | H2722 | 456 | 4.184817 | 0.699 | 0.769 | 0.85 | 0.8838 | 0.889 | 0.906 | 1.0135 | 0.9948 | 1.0129 |
| 026_1212_00300580 | Pleura | 1212 | H2810 | 456 | 4.445211 | 0.69 | 0.929 | 0.942 | 0.9243 | 0.9927 | 0.884 | 0.9289 | 0.9051 | 0.9626 |
| 026_682_00304600 | Pleura | 682 | NCI-H2452 | 456 | 4.452468 | 0.722 | 0.847 | 0.891 | 0.906 | 0.9407 | 0.899 | 0.9567 | 1.0786 | 1.1287 |
| 026_1200_00300690 | Pleura | 1200 | H2373 | 456 | 4.612295 | 0.695 | 0.911 | 0.887 | 0.9257 | 0.9258 | 0.849 | 0.9218 | 0.9772 | 0.9399 |
| 026_1214_00308460 | Pleura | 1214 | H2869 | 456 | 4.934927 | 0.775 | 0.881 | 0.878 | 0.926 | 0.9241 | 0.914 | 0.9475 | 0.9536 | 1.0138 |
| 026_1214_00308790 | Pleura | 1214 | H2869 | 456 | 4.961713 | 0.802 | 0.879 | 0.908 | 0.9132 | 0.9528 | 0.991 | 0.9959 | 1.0132 | 1.0202 |
| 026_1202_00303040 | Pleura | 1202 | H2591 | 456 | 5.067997 | 0.82 | 0.918 | 0.945 | 1.0327 | 0.9338 | 0.983 | 0.9541 | 1.0755 | 1.1707 |
| 026_1209_00300210 | Pleura | 1209 | H28 | 456 | 5.341766 | 0.817 | 0.821 | 0.827 | 0.8331 | 0.8999 | 0.927 | 0.9491 | 1.0072 | 0.9857 |
| 026_8078_00303060 | Pleura | 8078 | IST-MES1 | 456 | 5.358253 | 0.782 | 0.897 | 0.859 | 0.8822 | 0.8445 | 0.907 | 0.9752 | 0.926 | 1.0513 |
| 026_1199_00300560 | Pleura | 1199 | H2369 | 456 | 5.439636 | 0.82 | 0.91 | 0.99 | 0.8596 | 0.9184 | 0.984 | 0.9172 | 0.9896 | 1.1394 |
| 026_8078_00304970 | Pleura | 8078 | IST-MES1 | 456 | 5.505825 | 0.847 | 0.88 | 0.853 | 0.8587 | 0.8938 | 0.89 | 0.9416 | 0.9423 | 1.0968 |
| 026_1207_00298510 | Pleura | 1207 | H2731 | 456 | 5.56988 | 0.819 | 1.018 | 1.003 | 0.8986 | 0.9778 | 0.921 | 0.9543 | 0.9856 | 0.9695 |
| 026_1198_00302580 | Pleura | 1198 | H2052 | 456 | 5.579483 | 0.859 | 0.966 | 0.995 | 0.9551 | 0.9651 | 1.01 | 1.0265 | 1.0233 | 1.0095 |
| 026_1201_00302590 | Pleura | 1201 | H2461 | 456 | 5.654117 | 0.856 | 0.868 | 0.914 | 0.904 | 0.8852 | 0.908 | 0.9343 | 1.0239 | 1.1079 |
| 026_1213_00300720 | Pleura | 1213 | H2818 | 456 | 5.828097 | 0.843 | 0.946 | 0.86 | 0.9682 | 0.9236 | 0.942 | 0.9493 | 0.9688 | 0.9576 |
| 026_1211_00300570 | Pleura | 1211 | H2804 | 456 | 5.890027 | 0.881 | 0.886 | 0.907 | 0.8753 | 0.9153 | 0.955 | 0.969 | 0.9857 | 1.0448 |
| 026_1218_00300730 | Pleura | 1218 | H513 | 456 | 5.953268 | 0.9 | 0.905 | 0.905 | 1.0722 | 1.0284 | 0.976 | 0.9507 | 1.0341 | 1.2023 |
| 026_1208_00300710 | Pleura | 1208 | H2795 | 456 | 6.063798 | 0.3 | 0.943 | 0.961 | 0.8752 | 0.9823 | 0.905 | 0.9466 | 0.9643 | 0.975 |
| 026_1203_00300850 | Pleura | 1203 | H2595 | 456 | 6.184806 | 0.92 | 0.909 | 0.899 | 1.0357 | 1.0632 | 0.905 | 0.974 | 1.018 | 0.9037 |
| 026_8245_00282710 | pleural | 8245 | KMS-11 | 456 | 0.702427 | 0.356 | 0.381 | 0.404 | 0.4804 | 0.491 | 0.652 | 0.7128 | 1.0645 | 1.1048 |
| | effusion | | | | | | | | | | | | | |
| 026_996_00298490 | Prostate | 996 | 22RV1 | 456 | 2.346887 | 0.542 | 0.521 | 0.592 | 0.6234 | 0.6635 | 0.707 | 0.7764 | 0.8517 | 0.9168 |
| 026_985_00303070 | Prostate | 985 | LNCaP clone | 456 | 4.018762 | 0.727 | 0.863 | 0.923 | 1.1164 | 1.0922 | 0.984 | 1.0179 | 1.1749 | 1.0533 |
| | | | FGC | | | | | | | | | | | |
| 026_987_00298550 | Prostate | 987 | PC-3 | 456 | 4.097696 | 0.698 | 0.761 | 0.871 | 0.9338 | 0.9109 | 0.914 | 0.9938 | 1.2109 | 0.991 |
| 026_1001_0000200 | Prostate | 1001 | DU 145 | 456 | 5.044916 | 0.768 | 0.832 | 0.867 | 0.8699 | 0.8879 | 0.906 | 0.9933 | 0.9168 | 1.1136 |
| 026_988_00308270 | Prostate | 988 | PWR-1E | 456 | 5.228199 | 0.804 | 0.854 | 0.877 | 0.8566 | 0.8621 | 0.923 | 0.9727 | 0.9516 | 0.9806 |
| 026_997_00300660 | Prostate | 997 | BPH-1 | 456 | 5.448713 | 0.479 | 0.868 | 1.201 | 1.1209 | 1.1016 | 0.674 | 0.9562 | 1.1603 | 0.8983 |
| 026_1000_00300740 | Prostate | 1000 | VCaP | 456 | 5.968581 | 0.692 | 1.071 | 1.005 | 1.0988 | 0.9203 | 0.91 | 0.9225 | 0.9154 | 1.1301 |
| 026_1009_00264700 | Skin | 1009 | WM35 | 456 | −2.372512 | 0.112 | 0.201 | 0.203 | 0.2168 | 0.2138 | 0.242 | 0.3676 | 0.463 | 0.7025 |
| 026_8212_00264590 | Skin | 8212 | CP50-MEL-B | 456 | −1.651586 | 0.151 | 0.398 | 0.392 | 0.3817 | 0.3556 | 0.35 | 0.4631 | 0.5633 | 0.8209 |
| 026_1039_00265170 | Skin | 1039 | SK-MEL-30 | 456 | −1.476493 | 0.263 | 0.352 | 0.317 | 0.3236 | 0.3515 | 0.424 | 0.5534 | 0.8565 | 0.8549 |
| 026_1023_00269690 | Skin | 1023 | SK-MEL-2 | 456 | −1.357658 | 0.147 | 0.207 | 0.235 | 0.2621 | 0.2935 | 0.343 | 0.5053 | 0.6866 | 0.8936 |
| 026_1034_00264680 | Skin | 1034 | MEL-HO | 456 | −0.964561 | 0.144 | 0.372 | 0.375 | 0.3139 | 0.3537 | 0.327 | 0.4189 | 0.6188 | 0.8613 |
| 026_8073_00263480 | Skin | 8073 | HT-144 | 456 | −0.641233 | 0.154 | 0.241 | 0.236 | 0.3036 | 0.3695 | 0.397 | 0.6432 | 0.7444 | 0.9033 |
| 026_8114_00259970 | Skin | 8114 | MMAC-SF | 456 | −0.613008 | 0.211 | 0.399 | 0.492 | 0.3813 | 0.4238 | 0.662 | 0.5683 | 0.6942 | 1.0563 |
| 026_8209_00266540 | Skin | 8209 | A4-Fuk | 456 | −0.557176 | 0.179 | 0.476 | 0.467 | 0.4234 | 0.4176 | 0.498 | 0.6644 | 0.7338 | 0.7609 |
| 026_1046_00263470 | Skin | 1046 | HMVII | 456 | −0.454742 | 0.389 | 0.419 | 0.403 | 0.3903 | 0.4097 | 0.503 | 0.6117 | 0.8285 | 1.0625 |
| 026_8120_00262840 | Skin | 8120 | MZ7-mel | 456 | −0.254795 | 0.073 | 0.323 | 0.41 | 0.4001 | 0.4106 | 0.468 | 0.6687 | 0.6311 | 0.8761 |
| 026_8191_00260580 | Skin | 8191 | UACC-257 | 456 | −0.10266 | 0.456 | 0.468 | 0.426 | 0.404 | 0.4006 | 0.534 | 0.6654 | 0.7954 | 0.894 |
| 026_1176_00266430 | Skin | 1176 | 451Lu | 456 | −0.00066 | 0.26 | 0.428 | 0.38 | 0.386 | 0.4254 | 0.499 | 0.6795 | 0.8423 | 0.9729 |
| 026_1149_00263460 | Skin | 1149 | G-MEL | 456 | 0.018696 | 0.065 | 0.23 | 0.181 | 0.1928 | 0.2465 | 0.404 | 0.7707 | 0.8991 | 0.9576 |
| 026_1147_00263750 | Skin | 1147 | SK-MEL-28 | 456 | 0.142676 | 0.187 | 0.48 | 0.376 | 0.4566 | 0.4611 | 0.565 | 0.6083 | 0.8116 | 0.9418 |
| 026_1037_00260080 | Skin | 1037 | SK-MEL-1 | 456 | 0.168848 | 0.176 | 0.417 | 0.368 | 0.3716 | 0.3919 | 0.613 | 0.6978 | 0.9091 | 0.966 |
| 026_1190_00266570 | Skin | 1190 | Hs 939.T | 456 | 0.185332 | 0.283 | 0.631 | 0.615 | 0.5802 | 0.5603 | 0.559 | 0.5576 | 0.7187 | 0.8082 |
| 026_1024_00265150 | Skin | 1024 | M-14 | 456 | 0.192152 | 0.418 | 0.433 | 0.383 | 0.391 | 0.4651 | 0.618 | 0.742 | 0.7678 | 0.9177 |
| 026_1025_00269710 | Skin | 1025 | COLO-679 | 456 | 0.228168 | 0.267 | 0.288 | 0.296 | 0.3664 | 0.4611 | 0.681 | 0.8141 | 0.9334 | 1.1196 |
| 026_8161_00263740 | Skin | 8161 | SH-4 | 456 | 0.433257 | 0.203 | 0.383 | 0.33 | 0.3147 | 0.3323 | 0.488 | 0.7539 | 0.8959 | 0.983 |
| 026_8097_00262820 | Skin | 8097 | LB2518-MEL | 456 | 0.437866 | 0.076 | 0.501 | 0.37 | 0.3843 | 0.4237 | 0.706 | 0.7414 | 0.8914 | 0.9396 |
| 026_8120_00260550 | Skin | 8120 | MZ7-mel | 456 | 0.447706 | 0.299 | 0.428 | 0.435 | 0.4782 | 0.4839 | 0.498 | 0.6521 | 0.7326 | 0.8788 |
| 026_1033_00262470 | Skin | 1033 | IPC-298 | 456 | 0.460127 | 0.363 | 0.36 | 0.404 | 0.4297 | 0.5245 | 0.562 | 0.7868 | 0.9191 | 0.8529 |
| 026_1031_00264030 | Skin | 1031 | IGR-37 | 456 | 0.57267 | 0.159 | 0.498 | 0.512 | 0.4794 | 0.5367 | 0.602 | 0.8381 | 0.9754 | 1.0032 |
| 026_1006_00260110 | Skin | 1006 | WM-115 | 456 | 0.616077 | 0.079 | 0.437 | 0.583 | 0.4912 | 0.4928 | 0.627 | 0.6176 | 0.6485 | 0.9586 |
| 026_8023_00265291 | Skin | 8023 | COLO-829 | 456 | 0.740747 | 0.164 | 0.659 | 0.533 | 0.5534 | 0.5976 | 0.709 | 0.8699 | 0.9148 | 0.9877 |
| 026_1036_00264690 | Skin | 1036 | RVH-421 | 456 | 0.784255 | 0.099 | 0.511 | 0.489 | 0.5329 | 0.5785 | 0.531 | 0.5954 | 0.5867 | 0.7408 |
| 026_1011_00269770 | Skin | 1011 | WM278 | 456 | 0.944573 | 0.289 | 0.463 | 0.457 | 0.4545 | 0.4605 | 0.568 | 0.7513 | 0.8836 | 0.9597 |
| 026_1003_00269650 | Skin | 1003 | G-361 | 456 | 0.948854 | 0.099 | 0.326 | 0.434 | 0.5233 | 0.6155 | 0.705 | 0.83 | 0.8998 | 1.0756 |
| 026_8002_00263700 | Skin | 8002 | A101D | 456 | 0.960267 | 0.088 | 0.468 | 0.524 | 0.5105 | 0.5276 | 0.55 | 0.5835 | 0.7643 | 0.8791 |
| 026_8104_00260540 | Skin | 8104 | LOXIMVI | 456 | 1.072404 | 0.278 | 0.403 | 0.39 | 0.4769 | 0.6158 | 0.839 | 0.877 | 0.864 | 0.9209 |
| 026_1005_00268780 | Skin | 1005 | A-375 | 456 | 1.191442 | 0.45 | 0.499 | 0.485 | 0.4604 | 0.5149 | 0.506 | 0.6624 | 0.892 | 0.928 |
| 026_1004_00260870 | Skin | 1004 | C32 | 456 | 1.195832 | 0.288 | 0.331 | 0.256 | 0.265 | 0.3055 | 0.504 | 0.8497 | 0.9278 | 0.9753 |
| 026_1030_00264020 | Skin | 1030 | IGR-1 | 456 | 1.307792 | 0.075 | 0.388 | 0.556 | 0.5173 | 0.6922 | 0.688 | 0.8911 | 0.7819 | 0.8658 |
| 026_1035_00265160 | Skin | 1035 | MEL-JUSO | 456 | 1.414781 | 0.305 | 0.481 | 0.494 | 0.5153 | 0.7013 | 0.739 | 0.9127 | 0.9597 | 1.0093 |
| 026_8119_00259980 | Skin | 8119 | MZ2-MEL. | 456 | 1.446447 | 0.402 | 0.467 | 0.438 | 0.4246 | 0.4152 | 0.679 | 0.7345 | 0.8985 | 0.9484 |
| 026_8097_00260530 | Skin | 8097 | LB2518-MEL | 456 | 1.447504 | 0.439 | 0.438 | 0.44 | 0.3578 | 0.3688 | 0.635 | 0.8111 | 0.8622 | 0.8731 |
| 026_8225_00264640 | Skin | 8225 | SK-MEL-5 | 456 | 1.574212 | 0.07 | 0.432 | 0.581 | 0.5597 | 0.6534 | 0.603 | 0.7648 | 0.9285 | 1.0194 |
| 026_8104_00262830 | Skin | 8104 | LOXIMVI | 456 | 1.676415 | 0.172 | 0.565 | 0.594 | 0.5546 | 0.7085 | 0.865 | 1.2098 | 1.0454 | 1.1061 |
| 026_1042_00274340 | Skin | 1042 | COLO792 | 456 | 1.75025 | 0.329 | 0.514 | 0.573 | 0.6166 | 0.6518 | 0.787 | 0.8993 | 0.9066 | 0.9485 |
| 026_1026_00269720 | Skin | 1026 | COLO-783 | 456 | 1.791757 | 0.479 | 0.599 | 0.603 | 0.6144 | 0.5728 | 0.634 | 0.6979 | 0.8575 | 0.9077 |
| 026_1010_00262530 | Skin | 1010 | WM1552C | 456 | 2.109919 | 0.3664 | 0.794 | 0.685 | 0.6806 | 0.6737 | 0.881 | 0.8586 | 0.9992 | 0.9359 |
| 026_1041_00263450 | Skin | 1041 | A431 | 456 | 2.481335 | 0.436 | 0.563 | 0.633 | 0.821 | 0.8517 | 0.858 | 1.0792 | 0.8962 | 0.9346 |
| 026_8098_00269090 | Skin | 8098 | LB373-MEL-D | 456 | 2.623655 | 0.496 | 0.615 | 0.627 | 0.6225 | 0.6708 | 0.815 | 0.8109 | 0.9711 | 1.0201 |
| 026_1022_00262940 | Skin | 1022 | RPMI-7951 | 456 | 2.647594 | 0.249 | 0.552 | 0.678 | 0.6664 | 0.7181 | 0.824 | 0.8966 | 0.8605 | 0.908 |
| 026_1181_00269080 | Skin | 1181 | Hs 944.T | 456 | 2.824283 | 0.206 | 0.612 | 0.779 | 0.8499 | 0.8608 | 0.892 | 0.9924 | 1.1546 | 1.1214 |
| 026_1008_00262540 | Skin | 1008 | WM793B | 456 | 2.933974 | 0.371 | 0.744 | 0.693 | 0.6962 | 0.7393 | 0.731 | 0.8508 | 0.9586 | 0.8942 |
| 026_1027_00265280 | Skin | 1027 | COLO-800 | 456 | 3.132083 | 0.537 | 0.717 | 0.804 | 0.8959 | 0.9417 | 0.926 | 0.9678 | 0.954 | 1.0136 |
| 026_8034_00265300 | Skin | 8034 | DJM-1 | 456 | 3.234358 | 0.554 | 0.778 | 0.806 | 0.9452 | 0.9331 | 1.005 | 0.9966 | 1.0061 | 1.0084 |
| 026_1181_00264620 | Skin | 1181 | Hs 944.T | 456 | 3.491747 | 0.094 | 0.698 | 0.822 | 0.9152 | 0.8448 | 0.9 | 0.9921 | 0.9743 | 0.9841 |
| 026_8225_00263510 | Skin | 8225 | SK-MEL-5 | 456 | 3.505326 | 0.234 | 0.653 | 0.694 | 0.7331 | 0.7681 | 0.79 | 0.9216 | 0.9437 | 0.9528 |
| 026_1145_00263720 | Skin | 1145 | CHL-1 | 456 | 3.811805 | 0.609 | 0.895 | 0.833 | 0.8778 | 0.9025 | 0.855 | 0.9286 | 0.9316 | 1.0458 |
| 026_8060_00265311 | Skin | 8060 | GAK | 456 | 4.2621 | 0.37 | 0.758 | 0.827 | 0.7891 | 0.8733 | 0.894 | 0.9899 | 0.9692 | 0.9911 |
| 026_1047_00265320 | Skin | 1047 | MEWO | 456 | 4.342961 | 0.779 | 0.931 | 0.98 | 0.9757 | 0.9449 | 0.991 | 1.0008 | 0.9996 | 1 |
| 026_1022_00260910 | Skin | 1022 | RPMI-7951 | 456 | 4.58823 | 0.257 | 0.748 | 0.801 | 0.8703 | 0.8751 | 0.884 | 0.9495 | 0.9173 | 0.9542 |
| 026_1038_00260090 | Skin | 1038 | SK-MEL-3 | 456 | 4.834702 | 0.151 | 0.731 | 0.777 | 0.7955 | 0.7955 | 0.827 | 0.8793 | 0.913 | 0.9361 |
| 026_1002_00260861 | Skin | 1002 | A2058 | 456 | 5.928358 | 0.795 | 0.897 | 0.85 | 0.894 | 0.8474 | 0.885 | 1.0053 | 0.8902 | 0.984 |
| 026_8025_00306491 | Skin | 8025 | CP66-MEL | 456 | 6.278644 | 0.914 | 0.888 | 1.059 | 0.9053 | 1.0334 | 0.961 | 0.8798 | 1.0258 | 0.9475 |
| 026_1120_00264061 | Skin | 1120 | UACC-62 | 456 | 6.317961 | 0.417 | 0.99 | 0.941 | 0.9531 | 0.9635 | 1.005 | 1.0535 | 1.0033 | 0.9903 |
| 026_1049_00269761 | Skin | 1049 | VMRC-MELG | 456 | 6.363031 | 0.904 | 0.991 | 0.949 | 0.974 | 0.9849 | 1.004 | 1.032 | 1.0282 | 1.0264 |
| 026_8077_00311281 | Skin | 8077 | IST-MEL1 | 456 | 6.486215 | 0.817 | 0.849 | 0.783 | 0.8212 | 0.8361 | 0.894 | 0.8285 | 0.9342 | 0.9134 |
| 026_1002_00262871 | Skin | 1002 | A2058 | 456 | 6.768535 | 0.494 | 1.023 | 0.918 | 0.9957 | 0.9334 | 0.899 | 0.9223 | 0.931 | 0.9502 |
| 026_1191_00266580 | Skin | 1191 | Hs 940.T | 456 | 7.017835 | 0.562 | 0.98 | 1.123 | 1.1931 | 0.9727 | 1.159 | 1.1334 | 1.0446 | 1.0643 |
| 026_8211_00306531 | Skin | 8211 | SK-MEL-24 | 456 | 7.240348 | 0.995 | 1.062 | 1.047 | 0.9379 | 1.0364 | 1.075 | 1.0521 | 1.0504 | 1.0137 |
| 026_1004_00262880 | Skin | 1004 | C32 | 456 | 7.407136 | 1.327 | 0.196 | 1.169 | 0.1356 | 1.1547 | 0.213 | 1.1935 | 0.7281 | 1.1468 |
| 026_8077_00263490 | Skin | 8077 | IST-MEL1 | 456 | 8.189893 | 1.25 | 1.21 | 1.184 | 1.1565 | 0.9453 | 0.931 | 1.1642 | 1.0861 | 1.0387 |
| 026_1076_00260350 | Stomach | 1076 | OCUM-1 | 456 | −1.975727 | 0.138 | 0.299 | 0.203 | 0.1441 | 0.1472 | 0.137 | 0.1598 | 0.2644 | 0.5747 |
| 026_1050_00262790 | Stomach | 1050 | AGS | 456 | 0.402284 | 0.198 | 0.304 | 0.337 | 0.4411 | 0.6201 | 0.452 | 0.6355 | 0.7729 | 1.0239 |
| 026_1070_00258920 | Stomach | 1070 | HSC-39 | 456 | 1.218484 | 0.163 | 0.38 | 0.469 | 0.5806 | 0.7048 | 0.892 | 1.16 | 0.922 | 1.1787 |
| 026_8193_00311240 | Stomach | 8193 | ECC10 | 456 | 1.328742 | 0.306 | 0.42 | 0.445 | 0.5445 | 0.7218 | 0.758 | 0.8813 | 0.9883 | 0.9993 |
| 026_1052_00255810 | Stomach | 1052 | SNU-1 | 456 | 1.425654 | 0.369 | 0.469 | 0.497 | 0.5342 | 0.5814 | 0.77 | 0.7984 | 0.8285 | 0.947 |
| 026_1056_00316590 | Stomach | 1056 | KATO III | 456 | 1.949149 | 0.309 | 0.531 | 0.622 | 0.6777 | 0.7504 | 0.912 | 0.9052 | 1.0407 | 1.0158 |
| 026_1060_00256830 | Stomach | 1060 | MKN45 | 456 | 2.285447 | 0.504 | 0.586 | 0.532 | 0.5224 | 0.5043 | 0.577 | 0.7757 | 0.8156 | 0.9028 |
| 026_1072_00258880 | Stomach | 1072 | 23132/87 | 456 | 2.907608 | 0.473 | 0.717 | 0.911 | 0.8748 | 1.0846 | 1.055 | 1.07 | 0.981 | 1.001 |
| 026_1060_00262910 | Stomach | 1060 | MKN45 | 456 | 2.913103 | 0.498 | 0.498 | 0.474 | 0.9422 | 0.5296 | 0.623 | 1.0575 | 0.9958 | 0.8867 |
| 026_1078_00263960 | Stomach | 1078 | IM-95 | 456 | 3.209661 | 0.348 | 0.625 | 0.723 | 1.042 | 0.7453 | 0.866 | 0.8746 | 0.9835 | 0.9734 |
| 026_1054_00308870 | Stomach | 1054 | SNU-16 | 456 | 3.34225 | 0.574 | 0.78 | 0.883 | 0.9578 | 0.9599 | 0.973 | 0.9861 | 1.0293 | 1.027 |
| 026_1064_00258960 | Stomach | 1064 | NUGC-3 | 456 | 3.516892 | 0.253 | 0.754 | 0.947 | 0.9636 | 0.9596 | 1.019 | 1.0426 | 1.0278 | 1.0027 |
| 026_1075_00308260 | Stomach | 1075 | NUGC-4 | 456 | 3.652276 | 0.617 | 0.827 | 0.703 | 0.9984 | 1.0102 | 1.068 | 1.1379 | 0.9929 | 1.1512 |
| 026_1065_00273570 | Stomach | 1065 | MKN7 | 456 | 3.792655 | 0.621 | 0.777 | 0.872 | 0.891 | 0.9122 | 0.904 | 0.9197 | 0.9359 | 0.9547 |
| 026_1067_00311560 | Stomach | 1067 | RERF-GC-1B | 456 | 4.166168 | 0.732 | 0.677 | 0.761 | 0.8438 | 0.781 | 0.877 | 0.8716 | 0.958 | 1.0772 |
| 26_1067_00314080 | Stomach | 1067 | RERF-GC-1B | 456 | 4.402339 | 0.708 | 0.833 | 0.831 | 0.8383 | 0.8843 | 0.854 | 0.9212 | 0.9276 | 1.1227 |
| 026_1068_00258930 | Stomach | 1068 | MKN28 | 456 | 4.480011 | 0.554 | 0.885 | 0.897 | 0.9579 | 0.9774 | 1.009 | 1.0156 | 1.0042 | 1.0326 |
| 026_1057_00271310 | Stomach | 1057 | Hs 746T | 456 | 4.481195 | 0.745 | 0.743 | 0.677 | 0.6977 | 0.6882 | 0.86 | 0.875 | 0.9503 | 1.0984 |
| 026_1060_00260890 | Stomach | 1060 | MKN45 | 456 | 4.604315 | 0.753 | 0.738 | 0.731 | 0.6895 | 0.6891 | 0.836 | 1.0727 | 1.0008 | 0.9928 |
| 026_1051_00269200 | Stomach | 1051 | FU97 | 456 | 4.755599 | 0.738 | 0.851 | 0.914 | 0.8985 | 0.8947 | 0.955 | 0.9241 | 0.9866 | 1.0457 |
| 026_8187_00316461 | Stomach | 8187 | TGBC11TKB | 456 | 4.931222 | 0.775 | 0.953 | 0.925 | 0.9227 | 0.9488 | 0.988 | 0.9649 | 0.9982 | 0.9763 |
| 026_1058_00263730 | Stomach | 1058 | NCI-N87 | 456 | 5.400356 | 0.754 | 0.824 | 0.808 | 0.8382 | 0.8486 | 0.868 | 0.9105 | 0.9132 | 0.9828 |
| 026_1064_00264040 | Stomach | 1064 | NUGC-3 | 456 | 6.53754 | 0.915 | 1.021 | 1.007 | 1.0316 | 0.9966 | 1.014 | 1.0431 | 0.9819 | 1.0068 |
| 026_1077_00264820 | Stomach | 1077 | SCH | 456 | 6.77844 | 0.466 | 1.271 | 1.195 | 1.2103 | 1.1255 | 0.977 | 0.9179 | 1.1315 | 1.1153 |
| 026_1051_00265140 | Stomach | 1051 | FU97 | 456 | 6.812551 | 0.149 | 0.752 | 0.783 | 0.8562 | 0.833 | 0.758 | 0.8748 | 0.8287 | 0.8438 |
| 026_8067_00258320 | Stomach | 8067 | GT3TKB | 456 | 7.007727 | 0.623 | 0.977 | 1.004 | 1.0575 | 1.0443 | 1.026 | 1.0598 | 1.0143 | 1.0267 |
| 026_1053_00258360 | Stomach | 1053 | SNU-5 | 456 | 7.265878 | 1 | 0.967 | 0.868 | 0.95 | 0.958 | 1.018 | 1.0763 | 1.0284 | 1.0105 |
| 026_1073_00258910 | Stomach | 1073 | HGC-27 | 456 | 7.474785 | 0.055 | 1.003 | 1.012 | 1.0288 | 1.0225 | 1.013 | 1.0331 | 0.9845 | 0.9972 |
| 026_8062_00266170 | Stomach | 8062 | GCIY | 456 | 7.70033 | 1.076 | 1.218 | 0.962 | 1.0422 | 0.9155 | 1.202 | 0.9466 | 0.9302 | 1.1001 |
| 026_1059_00256820 | Stomach | 1059 | MKN1 | 456 | 7.84127 | 1.023 | 1.223 | 1.097 | 1.1806 | 1.0334 | 0.924 | 1.0209 | 0.9691 | 0.9885 |
| 026_8216_00258330 | Stomach | 8216 | RF-48 | 456 | 8.041101 | 0.627 | 1.056 | 1.075 | 1.0873 | 1.0084 | 1.031 | 1.0144 | 1.008 | 0.9914 |
| 026_8143_00302630 | Testes | 8143 | NTERA-S-cl-D1 | 456 | 4.238289 | 0.709 | 0.74 | 0.781 | 0.8439 | 0.8942 | 0.971 | 0.9553 | 0.9264 | 1.0878 |
| 026_1081_00299690 | Testes | 1081 | NCC-IT-A3 | 456 | 4.69755 | 0.834 | 0.925 | 0.99 | 1.0022 | 1.0753 | 0.959 | 1.0664 | 0.9907 | 0.9674 |
| 026_1082_00300430 | Testes | 1082 | NEC8 | 456 | 5.794775 | 0.518 | 0.919 | 1.026 | 1.1263 | 1.0267 | 0.882 | 1.2293 | 0.8206 | 0.9481 |
| 026_1087_00311210 | Thyroid | 1087 | BHT-101 | 456 | 0.094631 | 0.409 | 0.402 | 0.405 | 0.4025 | 0.4677 | 0.606 | 0.749 | 0.9156 | 0.9734 |
| 026_1098_00252970 | Thyroid | 1098 | IHH-4 | 456 | 0.734299 | 0.193 | 0.418 | 0.365 | 0.5027 | 0.4309 | 0.608 | 0.6796 | 0.9903 | 0.9333 |
| 026_1093_00253040 | Thyroid | 1093 | TT2609-C02 | 456 | 1.438563 | 0.33 | 0.462 | 0.489 | 0.4674 | 0.6027 | 0.719 | 0.8623 | 0.9552 | 1.0031 |
| 026_1100_00252990 | Thyroid | 1100 | KMH-2 | 456 | 1.898194 | 0.142 | 0.5 | 0.577 | 0.6566 | 0.6074 | 0.682 | 0.7694 | 0.8888 | 0.9822 |
| 026_1085_00252920 | Thyroid | 1085 | 8505C | 456 | 2.3348 | 0.536 | 0.498 | 0.605 | 0.5369 | 0.584 | 0.788 | 0.7985 | 1.0523 | 0.937 |
| 026_1088_00252930 | Thyroid | 1088 | CAL-62 | 456 | 2.495611 | 0.451 | 0.785 | 0.606 | 0.8114 | 0.6653 | 0.836 | 1.0285 | 1.0053 | 0.944 |
| 026_1089_00261010 | Thyroid | 1089 | HTC-C3 | 456 | 2.538171 | 0.577 | 0.55 | 0.623 | 0.7757 | 0.5886 | 0.616 | 0.7161 | 0.7476 | 0.9625 |
| 026_1090_00311330 | Thyroid | 1090 | ML-1 | 456 | 2.64388 | 0.368 | 0.64 | 0.793 | 0.8005 | 0.8127 | 0.931 | 0.9783 | 0.9389 | 1.0355 |
| 026_1090_00253010 | Thyroid | 1090 | ML-1 | 456 | 3.121258 | 0.165 | 0.778 | 0.787 | 0.6376 | 0.6432 | 0.975 | 1.0524 | 0.7737 | 1.0765 |
| 026_1090_00308850 | Thyroid | 1090 | ML-1 | 456 | 3.18393 | 0.308 | 0.673 | 0.724 | 0.8112 | 0.8338 | 0.925 | 0.9016 | 0.8988 | 1.0407 |
| 026_1086_00255730 | Thyroid | 1086 | B-CPAP | 456 | 3.682081 | 0.171 | 0.695 | 0.763 | 0.7585 | 0.7239 | 0.856 | 0.9452 | 0.9473 | 1.0079 |
| 026_8020_00314210 | Thyroid | 8020 | CGTH-W-1 | 456 | 4.407018 | 0.694 | 0.874 | 0.871 | 0.8186 | 0.9478 | 0.965 | 0.9497 | 1.0206 | 1.0176 |
| 026_1084_00266520 | Thyroid | 1084 | 8305C | 456 | 4.442179 | 0.592 | 0.739 | 0.746 | 0.7517 | 0.73 | 0.793 | 0.8273 | 0.8894 | 0.8901 |
| 026_8213_00259200 | Thyroid | 8213 | TT | 456 | 4.648028 | 0.769 | 0.966 | 0.965 | 0.9831 | 0.9968 | 0.984 | 0.9765 | 0.9632 | 0.942 |
| 026_8082_00252980 | Thyroid | 8082 | K5 | 456 | 6.244906 | 0.27 | 0.935 | 0.007 | 0.9319 | 0.951 | 0.93 | 0.997 | 0.9506 | 0.9691 |
| 026_1099_00306470 | Thyroid | 1099 | ASH-3 | 456 | 6.262224 | 0.912 | 0.905 | 0.964 | 0.9293 | 0.932 | 0.965 | 0.9777 | 0.9947 | 1.0629 |
| 026_1094_00259110 | Thyroid | 1094 | FTC-133 | 456 | 6.573461 | 0.084 | 0.958 | 0.978 | 1.0951 | 1.0879 | 1.057 | 1.063 | 1.0254 | 0.8654 |
| 026_1092_00259170 | Thyroid | 1092 | S-117 | 456 | 6.767861 | 0.271 | 0.973 | 0.988 | 1.0669 | 1.039 | 1.047 | 1.018 | 1.0563 | 1.0542 |
| 026_1097_00259160 | Thyroid | 1097 | RO82-W-1 | 456 | 7.698514 | 0.685 | 1.079 | 1.053 | 1.0554 | 1.0411 | 1.049 | 1.0682 | 1.0349 | 1.0563 |
| 26_8036_00304360 | Urinary | 8036 | DSH1 | 456 | 1.200178 | 0.377 | 0.348 | 0.397 | 0.5545 | 0.7402 | 0.871 | 0.9778 | 0.9884 | 1.0288 |
| | Track | | | | | | | | | | | | | |
| 026_24_00298840 | Urinary | 24 | RT4 | 456 | 2.281802 | 0.504 | 0.543 | 0.577 | 0.6272 | 0.7473 | 0.889 | 1.0564 | 1.0628 | 1.0768 |
| | Track | | | | | | | | | | | | | |
| 026_18_00299720 | Urinary | 18 | RT-112 | 456 | 2.448355 | 0.479 | 0.581 | 0.587 | 0.6728 | 0.7768 | 0.797 | 0.8705 | 1.106 | 1.03 |
| | Track | | | | | | | | | | | | | |
| 026_24_00252540 | Urinary | 24 | RT4 | 456 | 2.668527 | 0.216 | 0.59 | 0.634 | 0.6994 | 0.7144 | 0.855 | 0.9368 | 0.9696 | 1.0194 |
| | Track | | | | | | | | | | | | | |
| 026_8036_00257170 | Urinary | 8036 | DSH1 | 456 | 2.728478 | 0.679 | 0.605 | 0.639 | 0.6941 | 0.7355 | 0.829 | 0.9411 | 1.0033 | 1.0113 |
| | Track | | | | | | | | | | | | | |
| 026_9_00298850 | Urinary | 9 | SW 780 | 456 | 2.896276 | 0.523 | 0.587 | 0.676 | 0.7538 | 0.7859 | 0.866 | 0.9594 | 0.9717 | 1.0348 |
| | Track | | | | | | | | | | | | | |
| 026_15_00316530 | Urinary | 15 | BFTC-905 | 456 | 3.218144 | 0.607 | 0.59 | 0.688 | 0.6971 | 0.8338 | 0.848 | 0.8978 | 1.0448 | 1.0799 |
| | Track | | | | | | | | | | | | | |
| 026_8_00298880 | Urinary | 8 | UM-UC-3 | 456 | 3.221517 | 0.557 | 0.72 | 0.789 | 0.8471 | 0.8599 | 0.995 | 1.0484 | 1.045 | 1.0264 |
| | Track | | | | | | | | | | | | | |
| 026_6_00303090 | Urinary | 6 | 5637 | 456 | 3.348554 | 0.553 | 0.725 | 0.744 | 0.7649 | 0.9313 | 0.918 | 0.9221 | 0.9563 | 1.0354 |
| | Track | | | | | | | | | | | | | |
| 026_6_00256650 | Urinary | 6 | 5637 | 456 | 3.837233 | 0.726 | 0.703 | 0.715 | 0.7187 | 0.7189 | 0.755 | 0.9193 | 0.965 | 1.0004 |
| | Track | | | | | | | | | | | | | |
| 026_8101_00256280 | Urinary | 8101 | LB831-BLC | 456 | 3.939929 | 0.504 | 0.835 | 0.952 | 1.0143 | 1.0014 | 1.01 | 0.9848 | 0.9739 | 0.9713 |
| | Track | | | | | | | | | | | | | |
| 026_11_00298870 | Urinary | 11 | T24 | 456 | 4.120815 | 0.697 | 0.744 | 0.763 | 0.8533 | 0.8788 | 0.941 | 0.9969 | 1.0222 | 1.1105 |
| | Track | | | | | | | | | | | | | |
| 026_19_00298860 | Urinary | 19 | SW-1710 | 456 | 4.713264 | 0.758 | 0.847 | 0.828 | 0.8254 | 0.8943 | 0.979 | 0.9968 | 1.0264 | 1.0955 |
| | Track | | | | | | | | | | | | | |
| 026_7_00302400 | Urinary | 7 | SCaBER | 456 | 4.780909 | 0.779 | 0.775 | 0.708 | 0.7824 | 0.8163 | 0.882 | 1.0463 | 0.9494 | 1.0621 |
| | Track | | | | | | | | | | | | | |
| 026_8101_00302620 | Urinary | 8101 | LB831-BLC | 456 | 4.856105 | 0.76 | 0.882 | 0.929 | 0.9235 | 0.9393 | 0.926 | 0.9386 | 0.9281 | 1.0507 |
| | Track | | | | | | | | | | | | | |
| 026_22_00298810 | Urinary | 22 | HT 1376 | 456 | 4.888331 | 0.823 | 0.879 | 1.047 | 0.8692 | 1.0283 | 0.988 | 1.0731 | 1.1432 | 1.097 |
| | Track | | | | | | | | | | | | | |
| 026_20_00299740 | Urinary | 20 | VM-CUB1 | 456 | 4.987745 | 0.751 | 0.753 | 0.758 | 0.8989 | 0.8726 | 0.877 | 0.9581 | 0.9471 | 0.9762 |
| | Track | | | | | | | | | | | | | |
| 026_14_00298780 | Urinary | 14 | 647-V | 456 | 5.049696 | 0.854 | 0.851 | 0.873 | 0.8859 | 0.9584 | 1.097 | 1.1389 | 1.2074 | 1.0083 |
| | Track | | | | | | | | | | | | | |
| 026_12_00299730 | Urinary | 12 | TCCSUP | 456 | 5.306108 | 0.821 | 0.876 | 0.897 | 0.9178 | 0.9315 | 0.962 | 0.9176 | 1.0469 | 1.0217 |
| | Track | | | | | | | | | | | | | |
| 026_13_00298770 | Urinary | 13 | 639-V | 456 | 5.658711 | 0.867 | 0.933 | 0.849 | 0.9317 | 0.8466 | 1.046 | 1.0223 | 1.1266 | 1.2765 |
| | Track | | | | | | | | | | | | | |
| 026_16_00298800 | Urinary | 16 | CAL-29 | 456 | 5.956916 | 0.811 | 0.834 | 0.909 | 0.8884 | 0.9073 | 0.924 | 0.956 | 0.9709 | 0.9841 |
| | Track | | | | | | | | | | | | | |
| 026_3_00308800 | Urinary | 3 | HT-1197 | 456 | 6.060787 | 0.877 | 0.935 | 0.984 | 0.8762 | 0.9541 | 0.87 | 1.0393 | 1.0266 | 1.0429 |
| | Track | | | | | | | | | | | | | |
| 026_17_00300420 | Urinary | 17 | KU-19-19 | 456 | 6.388593 | 0.216 | 0.953 | 0.975 | 0.9996 | 0.9834 | 0.744 | 0.9959 | 0.9777 | 1.0473 |
| | Track | | | | | | | | | | | | | |
| 026_10_00298820 | Urinary | 10 | J82 | 456 | 6.755266 | 0.951 | 0.965 | 0.925 | 0.9536 | 1.0393 | 1.06 | 1.0882 | 1.0657 | 1.037 |
| | Track | | | | | | | | | | | | | |
| 026_8154_00262510 | Uterus | 8154 | RL95-2 | 456 | 0.033885 | 0.116 | 0.241 | 0.307 | 0.315 | 0.5516 | 0.413 | 0.6863 | 0.814 | 0.8778 |
| 026_1116_00268860 | Uterus | 1116 | SNG-M | 456 | 2.236003 | 0.377 | 0.605 | 0.635 | 0.4653 | 0.7155 | 0.805 | 0.9708 | 1.0702 | 1.1724 |
| 026_8154_00280250 | Uterus | 8154 | RL95-2 | 456 | 2.655708 | 0.475 | 0.611 | 0.69 | 0.592 | 0.6865 | 0.677 | 0.826 | 0.8426 | 0.9723 |
| 026_8166_00269750 | Uterus | 8166 | SK-UT-1 | 456 | 2.769805 | 0.399 | 0.642 | 0.861 | 0.9305 | 0.8691 | 0.946 | 0.8217 | 0.867 | 0.9308 |
| 026_1115_00268850 | Uterus | 1115 | SKN | 456 | 3.232484 | 0.402 | 0.724 | 0.819 | 0.8345 | 0.9291 | 0.941 | 1.0001 | 0.9878 | 0.9814 |
| 026_1112_00268820 | Uterus | 1112 | Ishikawa | 456 | 3.572844 | 0.62 | 0.659 | 0.62 | 0.6814 | 0.702 | 0.72 | 0.9036 | 0.8807 | 1.0744 |
| | | | (Heraklio) | | | | | | | | | | | |
| | | | 02 ER- | | | | | | | | | | | |
| 026_1107_00311310 | Uterus | 1107 | MFE-296 | 456 | 3.590101 | 0.601 | 0.697 | 0.647 | 0.7074 | 0.7773 | 0.802 | 0.8186 | 0.9166 | 1.0105 |
| 026_1108_00308840 | Uterus | 1108 | MFE-319 | 456 | 3.953038 | 0.7 | 0.723 | 0.698 | 0.8033 | 0.8993 | 0.819 | 0.9773 | 1.1189 | 1.0369 |
| 026_1109_00269701 | Uterus | 1109 | COLO684 | 456 | 4.059524 | 0.645 | 0.818 | 0.925 | 0.873 | 0.8541 | 0.934 | 0.9505 | 0.9782 | 1.0023 |
| 026_1102_00268790 | Uterus | 1102 | AN3CA | 456 | 4.238238 | 0.395 | 0.808 | 0.815 | 0.8203 | 0.9291 | 0.817 | 0.9786 | 0.9815 | 0.9877 |
| 026_1108_00314330 | Uterus | 1108 | MFE-319 | 456 | 4.375739 | 0.745 | 0.784 | 0.794 | 0.8526 | 0.9259 | 0.907 | 1.0498 | 1.0643 | 1.1339 |
| 026_1113_00268840 | Uterus | 1113 | MES-SA | 456 | 4.83984 | 0.832 | 0.962 | 1.003 | 0.9833 | 0.9906 | 0.984 | 0.9981 | 1.0121 | 0.9684 |
| 026_1117_00268810 | Uterus | 1117 | HEC-1 | 456 | 5.215029 | 0.824 | 0.904 | 0.885 | 0.8856 | 0.9834 | 0.972 | 0.9217 | 1.0697 | 1.0963 |
| 026_1113_00318661 | Uterus | 1113 | MES-SA | 456 | 5.307659 | 0.807 | 0.995 | 0.947 | 0.9632 | 1.0122 | 1.033 | 1.0384 | 1.0103 | 0.9636 |
| 026_8206_00264270 | Uterus | 8206 | KLE | 456 | 5.888467 | 0.885 | 1 | 1.029 | 1.0338 | 1.0454 | 1.029 | 0.9964 | 1.0291 | 0.9684 |
| 026_1105_00308190 | Uterus | 1105 | ESS-1 | 456 | 6.082924 | 0.887 | 0.915 | 0.911 | 0.9349 | 0.8927 | 0.94 | 0.9007 | 0.9407 | 0.9198 |
| 026_1104_00306500 | Uterus | 1104 | EN | 456 | 7.302741 | 1.047 | 0.983 | 0.999 | 0.9204 | 1.0053 | 1.011 | 1.0162 | 1.0749 | 1.0308 |
| 026_1106_00306510 | Uterus | 1106 | MFE-280 | 456 | 7.686964 | 1.124 | 0.983 | 1 | 0.9683 | 1.0601 | 1.156 | 1.0799 | 1.0206 | 0.9018 |
| 026_8163_00302640 | Vulva | 8163 | SK-LMS-1 | 456 | 2.432701 | 0.479 | 0.526 | 0.621 | 0.7198 | 0.8786 | 0.991 | 1.0278 | 1.0369 | 1.1613 |
| 026_8173_00302660 | Vulva | 8173 | SW954 | 456 | 2.875791 | 0.539 | 0.622 | 0.644 | 0.6956 | 0.8294 | 0.866 | 1.072 | 1.0089 | 1.087 |
| 26_8174_00304380 | Vulva | 8174 | SW962 | 456 | 4.641057 | 0.756 | 0.704 | 0.824 | 0.7868 | 0.8056 | 0.875 | 0.851 | 0.96 | 1.0607 |
| 026_481_00302570 | Vulva | 481 | CAL-39 | 456 | 5.164144 | 0.803 | 0.856 | 0.841 | 0.8864 | 0.9389 | 0.924 | 0.983 | 0.9909 | 1.1127 |
| 026_8174_00306540 | Vulva | 8174 | SW962 | 456 | 6.896884 | 0.97 | 0.963 | 1.002 | 0.9267 | 1.0771 | 0.973 | 1.0027 | 1.0435 | 1.0588 |

### Example 12

### BVD-523 Demonstrates In Vivo Antitumor Activity in BRAF^{V600E}-Mutant Cancer Cell Line Xenograft Models

Based on our in vitro findings that BVD-523 reduced proliferation and induced apoptosis in a concentration-dependent manner, BVD-523 was administered by oral gavage to demonstrate its in vivo anti-tumor activity in models with MAPK/ERK-pathway dependency. Xenograft models of melanoma (cell line A375), and colorectal cancer (cell line Colo205), were utilized, both of which harbor a BRAFV600E mutation.

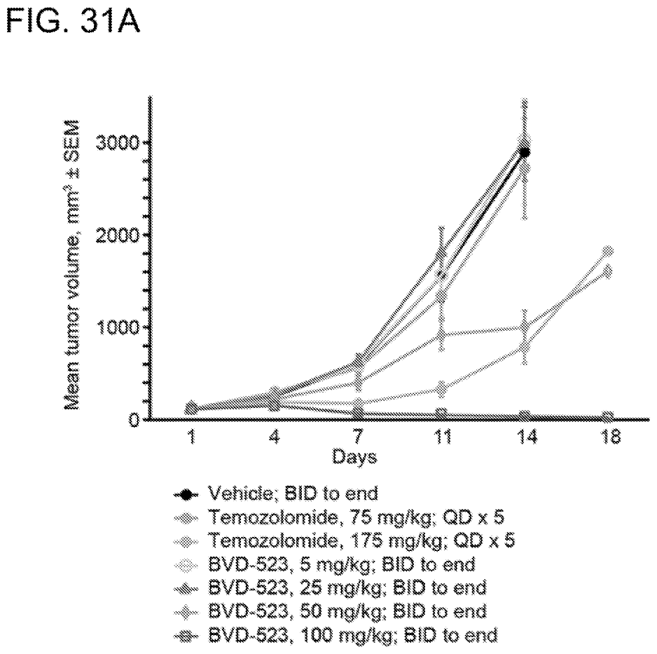

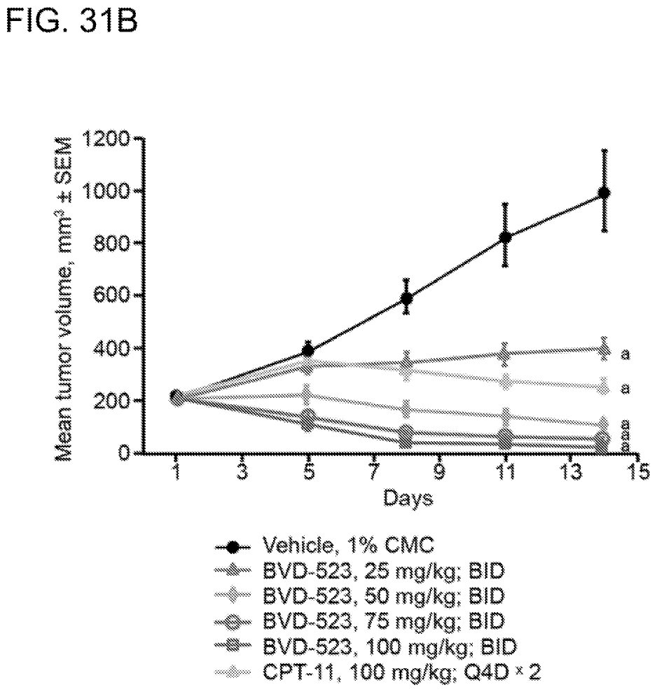

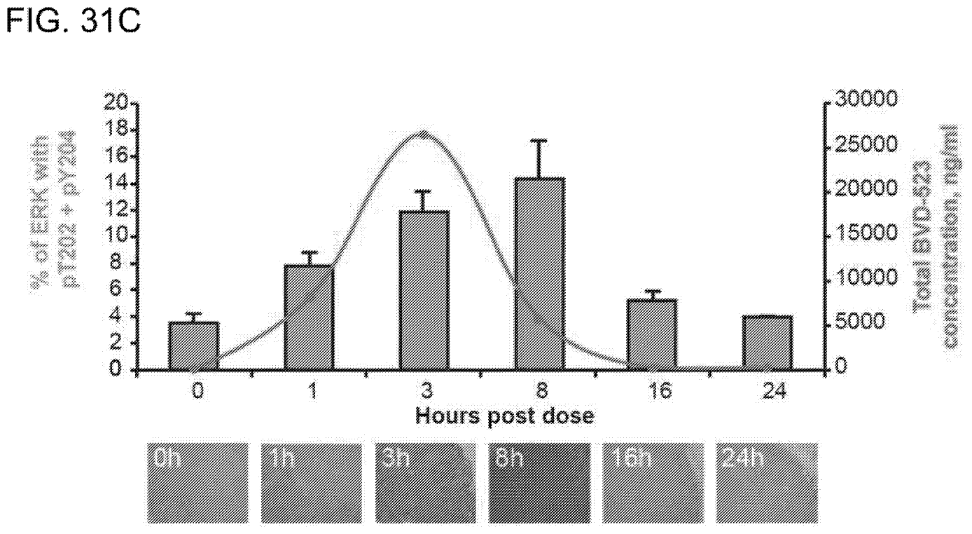

In A375 cell line xenografts, BVD-523 efficacy was compared with the control cytotoxic alkylating agent temozolomide following 14 days of treatment. BVD-523 demonstrated significant dose-dependent antitumor activity starting at 50 mg/kg twice daily (BID) (FIG. 31A). Doses of 50 and 100 mg/kg BID significantly attenuated tumor growth, with tumor growth inhibition (TGI) of 71% (P=0.004) and 99% (P<0.001), respectively. Seven partial regressions (PRs) were noted in the 100 mg/kg BID group; no regression responses were noted in any other group. The efficacy observed compared favorably with that of temozolomide, which when administered at 75 and 175 mg/kg resulted in modest dose-dependent TGI of 34% (P>0.05) and 78% (P=0.005), respectively.

Additionally, BVD-523 demonstrated antitumor efficacy in a Colo205 human colorectal cancer cell line xenograft model (FIG. 31B). BVD-523 again showed significant dose-dependent tumor regressions at doses of 50, 75, and 100 mg/kg BID, yielding mean tumor regressions T/Tᵢ (T=End of treatment, Tᵢ=Treatment initiation) of −48.2%, −77.2%, and −92.3%, respectively (all P<0.0001). Regression was not observed at the lowest dose of BVD-523 (25 mg/kg BID); however, significant tumor growth inhibition, with a T/C (T=Treatment, C=Control) of 25.2% (P<0.0001), was observed. Although not well tolerated, the positive control chemotherapeutic agent irinotecan (CPT-11) showed significant antitumor activity, inhibiting Colo205 tumor growth with a T/C of 6.4% (P<0.0001). However, even at its maximum tolerated dose in mice, CPT-11 was not as effective as BVD-523 at doses of 50, 75, or 100 mg/kg BID.

To establish the relationship between pharmacokinetics and pharmacodynamics, BVD-523 plasma concentrations were compared with pERK1/2 levels measured in the tumor by immunohistochemistry and isotope-tagged internal standard mass spectrometry over a 24-hour period following a single 100 mg/kg oral dose of BVD-523 (FIG. 31C). Phosphorylation of ERK1/2 was low in untreated tumors (0 hours). Following treatment with BVD-523, ERK1/2 phosphorylation steadily increased from 1 hour post-dose to maximal levels at 8 hours post-dose, then returned to pre-dose levels by 24 hours. This increase in pERK1/2 correlated with BVD-523 drug plasma concentrations. The in vivo observation of increased pERK1/2 with BVD-523 treatment is consistent with earlier in vitro findings (FIG. 30D).

### Example 13

### BVD-523 Results in ERK1/2 Substrate Inhibition Despite Increased ERK1/2 Phosphorylation

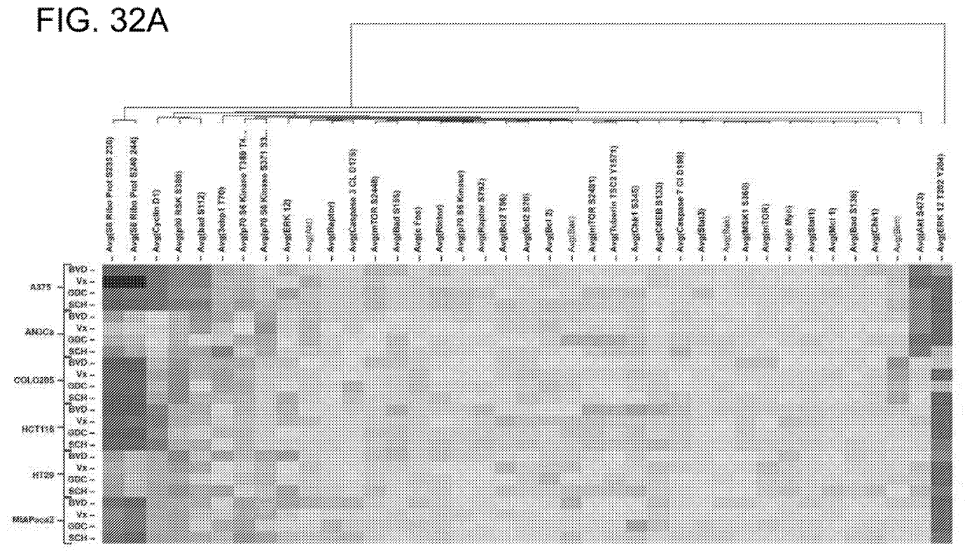

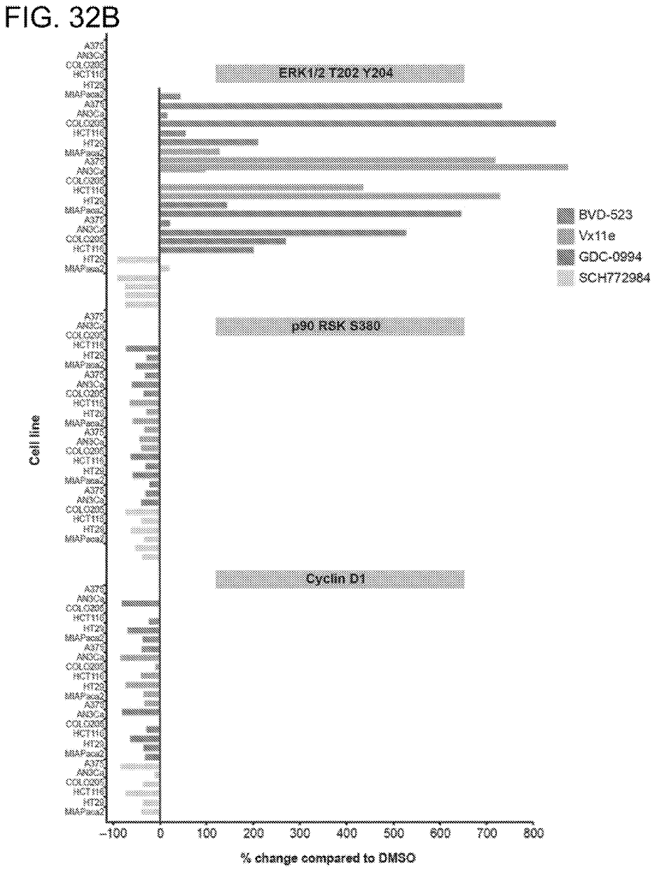

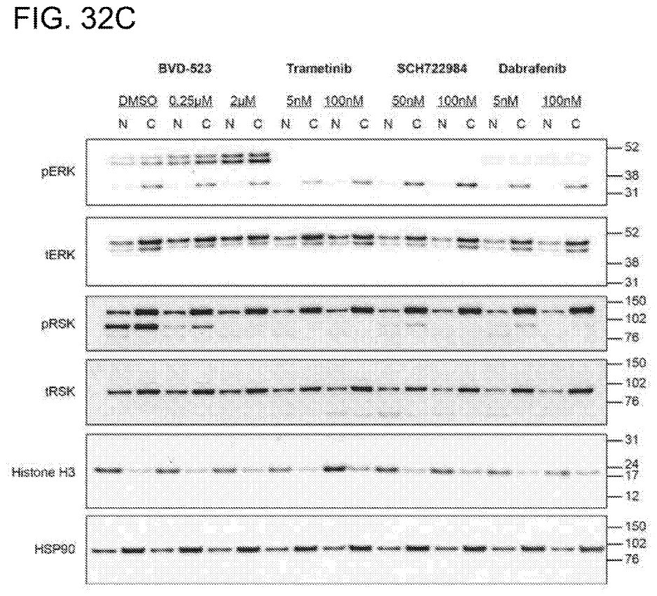

To examine the effects of BVD-523 on signaling relative to other known ERK1/2 inhibitors (SCH772984, GDC-0994, and Vx-Ile) (Morris et al. 2013 and Liu et al. 2015), a large-scale reverse phase protein array (RPPA) of approximately 40 proteins was employed in a variety of cell lines with sensitivity to ERK inhibition. Cell lines with common alterations in BRAF and RAS were assayed: BRAFV600E mutant lines A375, Colo205, and HT29; KRAS^{G12C}-mutant cell line MIAPACa-2; KRAS^{G13D}-mutant cell line HCT116; and AN3Ca with atypical HRAS^{F82L} mutation. Changes in protein levels are shown as a percentage change from dimethyl sulfoxide (DMSO)-treated parental control (FIG. 32A and Table 23). All ERK inhibitors elicited qualitatively similar protein effects, with the exception of phosphorylation of ERK1/2 (pERK1/2 [ERK1/2 -T202, -Y204]); SCH7722984 inhibited pERK1/2 in all cell lines, while BVD-523, GDC-0994, and Vx-Ile markedly increased pERK1/2. Phospho-p90 RSK (pRSK1) and cyclin D1, which are proximal and distal targets of pERK1/2, respectively, were similarly inhibited by all inhibitors tested regardless of the degree of ERK1/2 phosphorylation (FIG. 32B). These independent findings for BVD-523 are consistent with studies showing that phosphorylation of ERK1/2 substrates RSK1/2 remained inhibited despite dramatically elevated pERK1/2 by Western blots in A375 cells (FIG. 32D), in addition to protein-binding studies demonstrating BVD-523 binding and stabilization of pERK1/2 and inactive ERK1/2 (FIG. 29E and FIG. 29F). Therefore, measuring increased pERK1/2 levels could be considered as a clinical pharmacodynamic biomarker for BVD-523, while quantifying inhibition of ERK1/2 targets such as pRSK1 and DUSP6 as well could serve a similar purpose.

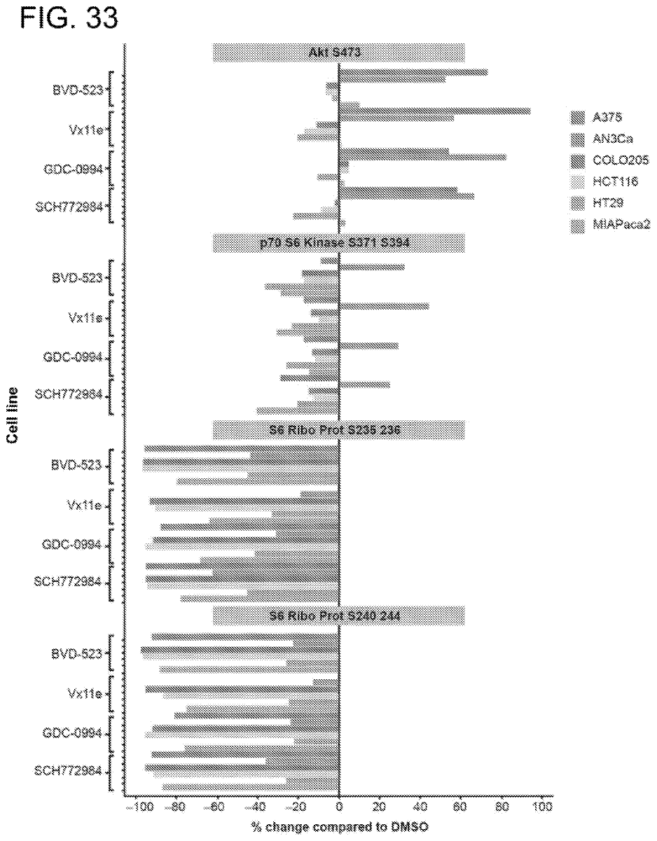

Additional protein changes are of note in this RPPA dataset (FIG. 32A). Decreased pS6-ribosomal protein appears to be another pharmacodynamic marker of ERK1/2 inhibition, as evidenced in all cell lines with all compounds (FIG. 32B). Furthermore, prominent induction of pAKT appears to be a cell line-dependent observation, where each ERK1/2 inhibitor induced pAKT in cell lines A375 and AN3CA cells (FIG. 33). Interestingly, the degree of inhibition of survival marker pBAD appears to differ between compounds, with only modest inhibition of pBAD by GDC-0994 compared with the other ERK1/2 inhibitors tested (FIG. 32A).

Next, how BVD-523 affects cellular localization of ERK1/2 and downstream target pRSK in a BRAF^{V600E}-mutant RKO colorectal cell line (FIG. 32C) was investigated. In resting cells, ERK1/2 localizes to the cytoplasm, and once stimulated pERK1/2 migrates to target organelles, particularly the nucleus where transcriptional targets are activated (Wainstein et al. 2016). In DMSO-treated control cells, pERK1/2 is evident in both nuclear and cytoplasmic fractions, which is likely reflective of MAPK pathway activity due to the presence of BRAFV600E in this cell line. Treatment with BVD-523 resulted in elevated pERK1/2 in the nucleus and cytoplasm as well as a modest increase in nuclear total ERK1/2 compared with DMSO-treated cells, suggesting that compound-induced stabilization of pERK1/2 stimulates some nuclear translocation. Despite increased pERK1/2 in both compartments, pRSK levels are lower in the cytoplasmic and nuclear compartments compared with DMSO control. Comparator MAPK signaling inhibitors (i.e., trametinib, SCH7722984, dabrafenib) inhibited phosphorylation of ERK1/2 and RSK, as reflected by lower levels in the nuclear and cytoplasmic compartments. These data again suggest that BVD-523-associated increases in pERK1/2 are evident in both the cytoplasm and nucleus; however, this does not translate to activation of target substrates. This is consistent with data presented in FIG. 30D and FIG. 32A.

TABLE 23

| | | % change from DMSO (matched cell line) | | | | | |
|---|---|---|---|---|---|---|---|
| | | Avg (S6 | Avg (S6 | | | | |
| | | Ribo Prot | Ribo Prot | Avg | Avg (p90 | Avg | Avg |
| Cell Line | Treatment | S235 236) | S240 244) | (Oyclin D1) | RSK S380) | (bad S112) | (4ebp1 T70) |
| A375 | BVD | −95.3 | −91.98 | −81.45 | −71 | −72.37 | −31.82 |
| A375 | Vx | (Empty) | (Empty) | −85.46 | −65.25 | −69.29 | 23.33 |
| A375 | GDC | −87.61 | −80.3 | −81.65 | −60.74 | −55.32 | −29.47 |
| A375 | SCH | −94.71 | −91.78 | −84.05 | −72.44 | −71.44 | −31.75 |
| AN3Ca | BVD | −43.41 | −22.2 | −0.69 | −28.11 | −54.1 | 32.17 |
| AN3Ca | Vx | −18.54 | −12.55 | −9.59 | −28.41 | −45.63 | 16.67 |
| AN3Ca | GDC | −30.74 | −23.47 | 0.34 | −29.44 | 2.53 | 11.28 |
| AN3Ca | SCH | −61.99 | −35.88 | −11.33 | −40.26 | −39.14 | 61.57 |
| COLO205 | BVD | −96.15 | −97.33 | −23.65 | −50.84 | −31.51 | −36.35 |
| COLO205 | Vx | −93.04 | −94.89 | −39.79 | −58.6 | −30.28 | −43.71 |
| COLO205 | GDC | −91.19 | −91.59 | −28.02 | −57.5 | −6.12 | −36.69 |
| COLO205 | SCH | −94.67 | −95.09 | −36.7 | −62.31 | −29.4 | −27.51 |
| HCT116 | BVD | −96.31 | −96.26 | −69.62 | −31.81 | −34.27 | 4.03 |
| HCT116 | Vx | −90.03 | −86.06 | −72.72 | −33.05 | −23.88 | 10.35 |
| HCT116 | GDC | −94.82 | −95.1 | −63.59 | −22.25 | −12.36 | 20.5 |
| HCT116 | SCH | −93.86 | −91.07 | −73.21 | −33.7 | −31.29 | 5.6 |
| HT29 | BVD | −44.68 | −25.67 | −37.21 | −60.5 | −20.66 | −41.47 |
| HT29 | Vx | −32.8 | −24.35 | −35.2 | −43.41 | −35.62 | −2.89 |
| HT29 | GDC | −41.45 | −21.74 | −35.69 | −30.59 | −12.98 | 1.95 |
| HT29 | SCH | −44.9 | −25.73 | −36.66 | −53.88 | −33.9 | −40.58 |
| MIAPaca2 | BVD | −79.46 | −88.03 | −37.9 | −35 | −30.29 | −9.42 |
| MIAPaca2 | Vx | −63.36 | −74.82 | −33.96 | −39.91 | −20.85 | −15.72 |
| MIAPaca2 | GDC | −67.9 | −75.59 | −31.92 | −39.09 | −10.08 | −34.01 |
| MIAPaca2 | SCH | −77.57 | −86.61 | −39.88 | −38.58 | −33.07 | 19.27 |
| | | % change from DMSO (matched cell line) | | | | | |
| | | Avg | Avg | | | | |
| | | (p70 S6 | (p70 S6 | | | | |
| | | Kinase | Kinase | Avg | Avg | Avg | Avg (Caspase |
| Cell Line | Treatment | T389 T412) | S371 S394) | (ERK 12) | (Akt) | (Raptor) | 3 CL D175) |
| A375 | BVD | −23.79 | −8.58 | −22.91 | −14.03 | −7.77 | −8.06 |
| A375 | Vx | −25.54 | −17.32 | −5.39 | −30.34 | −12.7 | −9.27 |
| A375 | GDC | −31.9 | −17.34 | 31.55 | −20.7 | −14.32 | −16.74 |
| A375 | SCH | −42.73 | −28.72 | −21.65 | −23.26 | −11.66 | −9.87 |
| AN3Ca | BVD | −14.78 | 32.26 | −9.05 | −22.43 | −13.82 | −10.8 |
| AN3Ca | Vx | 0.56 | 44.04 | −11.27 | −24.62 | −2.47 | −12.7 |
| AN3Ca | GDC | 26.01 | 29.09 | −2.87 | −26.04 | −8.05 | 1.55 |
| AN3Ca | SCH | −16.63 | 24.56 | −9.27 | −16.35 | −11.09 | 1.25 |
| COLO205 | BVD | −36.4 | −18.11 | −18.83 | −3.85 | −7.14 | −3.18 |
| COLO205 | Vx | −28 | −13.64 | −12.32 | −12.51 | −0.05 | −2.67 |
| COLO205 | GDC | −32.2 | −13.02 | −3.33 | −11.83 | −5.48 | 23.06 |
| COLO205 | SCH | −30.4 | −14.59 | −31.87 | −10.31 | −2.2 | 14.08 |
| HCT116 | BVD | −28.11 | −16.9 | −29.42 | 4.41 | −7.06 | −10.11 |
| HCT116 | Vx | −20.99 | −9.89 | −24.01 | −18.15 | −4.32 | −5.19 |
| HCT116 | GDC | −24.73 | −11.47 | −1.9 | −6.13 | −6.2 | −8.36 |
| HCT116 | SCH | −24.63 | −12.3 | −10.22 | −9.86 | −9.66 | −4.63 |
| HT29 | BVD | −24.58 | −35.94 | −44.3 | −13.41 | −8.53 | −7.03 |
| HT29 | Vx | −12.31 | −22.86 | 0.24 | −17.84 | −6.53 | −2.86 |
| HT29 | GDC | −20.86 | −25.73 | 4.66 | −10.01 | −6.85 | −3.44 |
| HT29 | SCH | −9.55 | −20.52 | −37 | −16.93 | −12.18 | −7.9 |
| MIAPaca2 | BVD | −39.23 | −28.27 | −40.33 | 23.63 | 21.15 | 22.35 |
| MIAPaca2 | Vx | −30.66 | −30.35 | −14.85 | −0.15 | 5.4 | 6.17 |
| MIAPaca2 | GDC | −40.99 | −14.4 | −6.88 | 4.33 | 22.43 | 10.47 |
| MIAPaca2 | SCH | −50.97 | −40.47 | −23.09 | 13.47 | 17.66 | 21.05 |

| | | % change from DMSO (matched cell line) | | | | | | |
|---|---|---|---|---|---|---|---|---|
| | | Avg | Avg | Avg | Avg | Avg (p70 | Avg | Avg |
| Cell Line | Treatment | (mTOR S2448) | (Bad S155) | (cFos) | (Rictor) | S6 Kinase) | (Raptor S792) | (Bcl 2 T56) |
| A375 | BVD | −27.87 | −21.9 | 4.39 | −20.11 | −7.6 | −10.33 | −8.54 |
| A375 | Vx | −21.66 | −6.3 | −13.65 | −8.16 | −6.42 | −0.86 | −7.53 |
| A375 | GDC | −23.61 | −13.31 | −12.46 | −23.29 | −18.11 | −15.93 | −4.1 |
| A375 | SCH | −26.17 | −13.86 | −12.51 | −22.13 | −17.66 | −6.89 | −19.55 |
| AN3Ca | BVD | −10.79 | 0.66 | −5.15 | −4.52 | −10.27 | −8.47 | −12.85 |
| AN3Ca | Vx | −2.37 | 4.59 | −5.52 | 0.02 | −2.33 | 0.37 | −11.73 |
| AN3Ca | GDC | −2.96 | 17.31 | −0.63 | −9.21 | −3.67 | 4.85 | 1.23 |
| AN3Ca | SCH | −4.84 | 12.92 | −9.18 | −10.89 | −7.71 | −4.03 | −10.73 |
| COLO205 | BVD | −23.51 | −18.18 | −12.25 | −5.21 | 0.14 | 0.41 | −11.84 |
| COLO205 | Vx | −8.52 | −9.72 | −19.34 | 1.65 | −3.42 | 0.2 | −12.02 |
| COLO205 | GDC | −7.36 | −9.11 | −21.33 | −5.04 | 5.83 | −9.04 | −4.6 |
| COLO205 | SCH | −9.44 | −10.96 | −15.17 | −19.07 | −2.85 | −4.17 | −6.73 |
| HCT116 | BVD | −12.78 | −30.72 | −14.08 | −13.05 | −12.86 | −22.04 | −8.36 |
| HCT116 | Vx | −10.12 | −15.59 | −13.89 | 1.78 | −4.45 | −11.21 | −25 |
| HCT116 | GDC | −19.33 | −19.71 | −10.36 | −10.98 | −9.9 | −15.77 | −11.96 |
| HCT116 | SCH | −16.05 | −22.96 | −15.27 | −18.5 | −14.12 | −18.18 | −15.89 |
| HT29 | BVD | −20.68 | −25.9 | −13.48 | −18.76 | −10.64 | −10.48 | −8.18 |
| HT29 | Vx | −13.94 | −11.26 | −8.23 | −2.6 | −1.72 | −2.13 | −25.01 |
| HT29 | GDC | −11.44 | −6.7 | −12.92 | −10.62 | −2.83 | −1.75 | 1.61 |
| HT29 | SCH | −22.65 | −7.98 | −9.26 | −13.03 | −2.36 | −6.38 | −17.9 |
| MIAPaca2 | BVD | −11.73 | −5.65 | −18.44 | −11.11 | −3.59 | 5.95 | −1.78 |
| MIAPaca2 | Vx | −5.38 | 7.38 | −4.43 | −13.24 | −9.27 | −3.74 | −9.86 |
| MIAPaca2 | GDC | 11.84 | 8.18 | −16.12 | −11.25 | −3.25 | 2.18 | −2.12 |
| MIAPaca2 | SCH | −11.48 | −1.71 | −8.99 | −15.15 | −7.51 | 4.43 | −1.98 |
| | | % change from DMSO (matched cell line) | | | | | | |
| | | | | | Avg | Avg | | |
| | | Avg | Avg | Avg | (mTOR | (Tuberin | Avg | Avg |
| Cell Line | Treatment | (Bcl2 S70) | (Bcl2) | (Bax) | S2481) | TS02 Y1571) | (Chk1 S345) | (CREB S133) |
| A375 | BVD | −7.91 | −12.55 | −6.98 | −3.36 | −6.32 | −7.43 | −12.65 |
| A375 | Vx | −4.02 | −9.93 | −5.05 | −2.9 | −3.55 | 0.56 | −15.8 |
| A375 | GDC | 6.3 | −3.13 | 3.97 | −19.37 | −16.74 | −18.35 | −1.18 |
| A375 | SCH | −12.91 | −18.53 | −17.32 | −15.82 | −13.7 | −10.59 | −1.55 |
| AN3Ca | BVD | −14.81 | −16.83 | −10.47 | 2.75 | 2.86 | −13.34 | 1.03 |
| AN3Ca | Vx | −14.39 | −18.77 | −7.89 | 7.22 | 3.8 | −9.54 | 0.16 |
| AN3Ca | GDC | −0.65 | 7.8 | 23.83 | 25.42 | 25.18 | 15.46 | 5.03 |
| AN3Ca | SCH | −12.42 | −5.14 | 0.15 | 11.24 | 13.44 | 14.56 | 3.64 |
| COLO205 | BVD | −10.2 | −0.13 | 0.28 | −1.33 | −3.48 | −4.63 | −2.67 |
| COLO205 | Vx | −20.82 | −14.09 | −9.16 | −9.43 | −9.45 | 13.12 | −10.64 |
| COLO205 | GDC | −9 | 17.95 | 8.2 | 0.96 | 0.66 | 6.08 | −11.85 |
| COLO205 | SCH | −18.52 | −14.56 | −6.44 | −6.68 | −1.38 | 1 | −14.56 |
| HCT116 | BVD | −8.23 | −3.59 | −0.8 | −34.09 | −29.89 | −39.65 | −30.64 |
| HCT116 | Vx | −17.79 | −17.3 | −20.46 | −17.82 | −16.51 | −16.49 | −9.17 |
| HCT116 | GDC | −12.53 | −4.56 | −20.11 | −18.34 | −9.4 | −18.77 | −8.56 |
| HCT116 | SCH | −18.34 | −10.58 | −13.86 | −22.53 | −16.17 | −18.51 | −24.18 |
| HT29 | BVD | 2.83 | 10.68 | 8.39 | −8.64 | 0.66 | −13.48 | −3.46 |
| HT29 | Vx | −12.55 | −16.73 | −18.47 | 0.52 | 2.85 | 6.44 | 14.16 |
| HT29 | GDC | 10.59 | 18.16 | 1.1 | 3.23 | 11.86 | 6.88 | 9.54 |
| HT29 | SCH | −11.45 | −10.01 | −20.59 | 13.93 | 13.25 | 19.05 | 9.87 |
| MIAPaca2 | BVD | 0.89 | 5.86 | 23.44 | −0.24 | 1.7 | 1.2 | −19.26 |
| MIAPaca2 | Vx | −7.83 | −2.3 | 4.33 | −3.12 | −0.01 | 2.09 | −13.28 |
| MIAPaca2 | GDC | −6.78 | −7.8 | 3.48 | 3.65 | 5.71 | 32.96 | −21.37 |
| MIAPaca2 | SCH | −4.23 | 4.96 | 15.67 | 2.43 | 8.86 | 13.39 | −6.63 |

| | | % change from DMSO (matched cell line) | | | | | | | |
|---|---|---|---|---|---|---|---|---|---|
| | | Avg | | | | | | | |
| | | (Caspase 7 | Avg | Avg | Avg | Avg | Avg | Avg | Avg |
| Cell Line | Treatment | CI D198) | (Stat3) | (Bak) | (MSK1 S360) | (mTOR) | (cMyc) | (Stat1) | (Mcl1) |
| A375 | BVD | 3.18 | 6.02 | −3.26 | 3.35 | 0.2 | 2.56 | 1.32 | 10.67 |
| A375 | Vx | −7.62 | 1.71 | 0.65 | −7.63 | −3.31 | −0.97 | 0.23 | 1.6 |
| A375 | GDC | −5.53 | 14.24 | 2.76 | −5.21 | −6.2 | −9.11 | −5.67 | −9.09 |
| A375 | SCH | −6 | −1.42 | −2.37 | 0.97 | −5.18 | 1.86 | −6.01 | 2.11 |
| AN3Ca | BVD | −12.35 | −4.97 | 3.38 | 0.49 | 5.58 | −0.58 | −5.65 | −4.9 |
| AN3Ca | Vx | −8.09 | −7.67 | −1.6 | 6.83 | −6.27 | −5.47 | −0.83 | −4.24 |
| AN3Ca | GDC | −15.56 | −13.05 | 7.33 | 6.84 | −2.85 | 3.99 | 9.15 | −4.16 |
| AN3Ca | SCH | −24.17 | −6.01 | 9.73 | −5.02 | −5.33 | 0.88 | 5.52 | −7.15 |
| COLO205 | BVD | 6.76 | −2.58 | 3.79 | 14.69 | 12.77 | −1.11 | 0.53 | −2.7 |
| COLO205 | Vx | 0.03 | −7.96 | −2.79 | 6.22 | 3.62 | −7.42 | −6.1 | −10.64 |
| COLO205 | GDC | 1.37 | −0.86 | 6.58 | 0.82 | 3.39 | −6.22 | 7.26 | 2.5 |
| COLO205 | SCH | 12.36 | −6.1 | 4.59 | 7.54 | −5.62 | −0.64 | −4.31 | −0.14 |
| HCT116 | BVD | −21.09 | −5.52 | −13.16 | −13.45 | −11.96 | −11.15 | −13.76 | −8.72 |
| HCT116 | Vx | −11.41 | −7.76 | −3.07 | −13.73 | −0.59 | 18.56 | −14.06 | −7.79 |
| HCT116 | GDC | −13.99 | −0.25 | −1.74 | −12.93 | −4.85 | 12.03 | −6.32 | −4.77 |
| HCT116 | SCH | −16.35 | −7.67 | −4.66 | −15.32 | −12.56 | 12.86 | −10.74 | −6.55 |
| HT29 | BVD | −6.93 | −6.46 | −1.42 | 1.49 | 5.02 | 17.85 | 4.74 | −2.83 |
| HT29 | Vx | 8.28 | −4.28 | 2.69 | 5.64 | 16.94 | 10.71 | 4.29 | 1.36 |
| HT29 | GDC | −0.88 | −3.69 | 3.56 | −0.04 | 10.45 | 7.56 | 2.58 | 3.71 |
| HT29 | SCH | 7.02 | −2.15 | 2.78 | 1.25 | 9.31 | 13.55 | 5.79 | 14.05 |
| MIAPaca2 | BVD | −6.44 | −1.23 | −15.15 | −10.72 | −3.27 | −8.77 | 8.62 | −7.27 |
| MIAPaca2 | Vx | −0.32 | 0.61 | −1.93 | −0.87 | −1.58 | −5.94 | −1.64 | −6.58 |
| MIAPaca2 | GDC | −4.92 | −9.44 | −8.11 | −5.25 | −9.8 | −7.84 | 7.4 | −1.79 |
| MIAPaca2 | SCH | 8.35 | −9.19 | −10.88 | 2.74 | 1.64 | 2.11 | 7.17 | −1.88 |

| | | % change from DMSO (matched cell line) | | | | |
|---|---|---|---|---|---|---|
| | | | | | | Avg |
| | | Avg | Avg | Avg | Avg | (ERK 1 2 |
| Cell Line | Treatment | (Bad S136) | (Chk1) | (Bim) | (Akt S473) | T202 Y204) |
| A375 | BVD | 12.31 | 16.94 | 14.57 | 73.07 | 43.34 |
| A375 | Vx | 5.06 | −0.86 | 12.32 | 93.85 | 128.93 |
| A375 | GDC | −6.21 | −7.72 | 9.8 | 53.66 | 142.37 |
| A375 | SCH | 5.32 | 2.83 | 17.52 | 58.13 | −90.63 |
| AN3Ca | BVD | −1.02 | −8.45 | −12.57 | 52.11 | 733.27 |
| AN3Ca | Vx | −3.81 | −0.46 | −1.46 | 56.17 | 718.94 |
| AN3Ca | GDC | 0.71 | 9.43 | −11.63 | 82.37 | 645.5 |
| AN3Ca | SCH | 5.26 | 1.35 | −14.09 | 66.17 | 19.75 |
| COLO205 | BVD | 5.05 | −2.86 | 41.73 | −5.78 | 14.39 |
| COLO205 | Vx | −1.01 | −9.16 | 34.1 | −10.96 | 98.48 |
| COLO205 | GDC | 4 | 5.06 | 20.59 | 4.45 | 20.01 |
| COLO205 | SCH | 1.88 | 7.49 | 29.22 | −1.74 | −91.43 |
| HCT116 | BVD | −11.49 | −11.26 | 12.44 | −6.14 | 849.12 |
| HCT116 | Vx | −5.39 | −8.63 | 4.82 | −16.94 | 873.33 |
| HCT116 | GDC | −1.24 | 2.28 | 6.2 | 4.69 | 526.64 |
| HCT116 | SCH | −10.55 | −5.58 | 1.95 | −8.76 | −75.21 |
| HT29 | BVD | 10.73 | 7.4 | 2.81 | −3.06 | 54.82 |
| HT29 | Vx | 5.53 | 4.68 | 3.12 | −20.5 | 435.68 |
| HT29 | GDC | 2 | 5.68 | 7.74 | −10.5 | 268.99 |
| HT29 | SCH | 9.67 | 14.64 | 0.69 | −22.14 | −74.84 |
| MIAPaca2 | BVD | −4.12 | −3.51 | −12.51 | 9.9 | 209.14 |
| MIAPaca2 | Vx | 0.36 | −1.8 | 2.1 | 0.48 | 729.27 |
| MIAPaca2 | GDC | 2.25 | 6.56 | 5.07 | 2.24 | 199.59 |
| MIAPaca2 | SCH | 8.62 | 12.07 | 2.08 | 2.84 | −76.71 |

### Example 14

### BVD-523 Exhibited Activity in In Vitro Models of BRAF and MEK Inhibitor Resistance

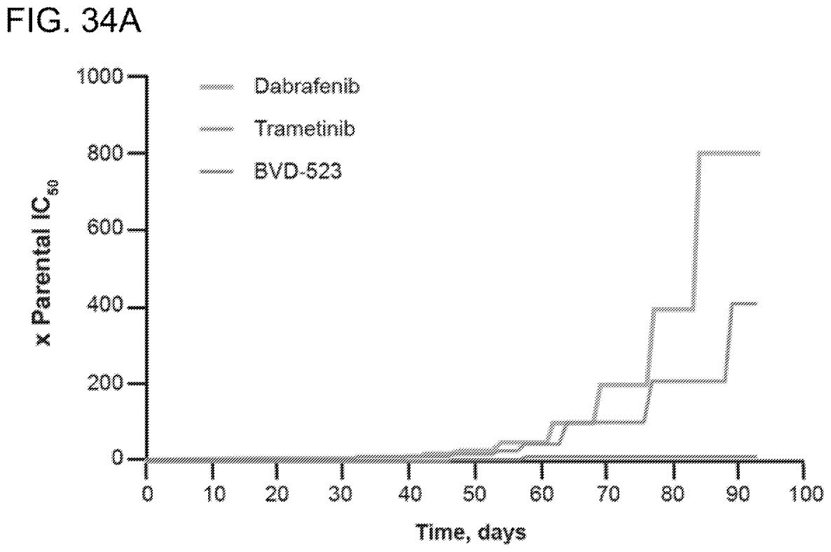

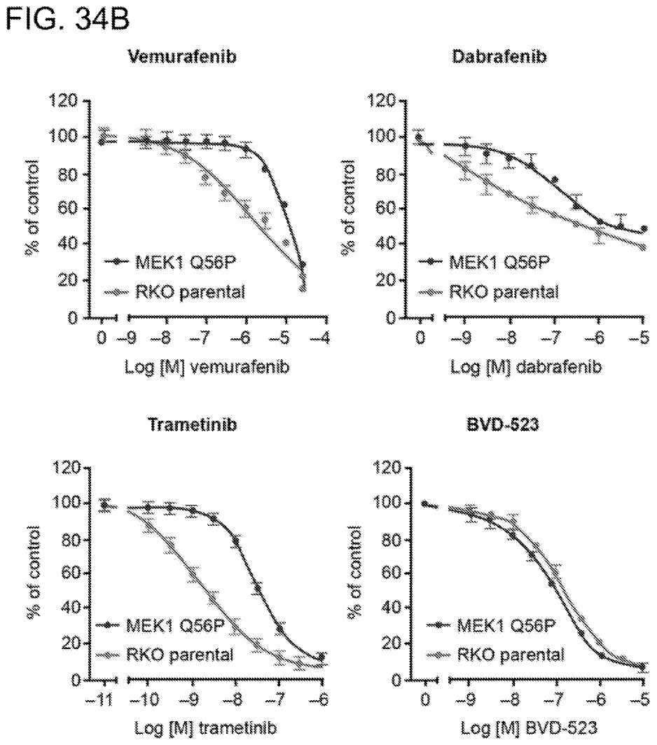

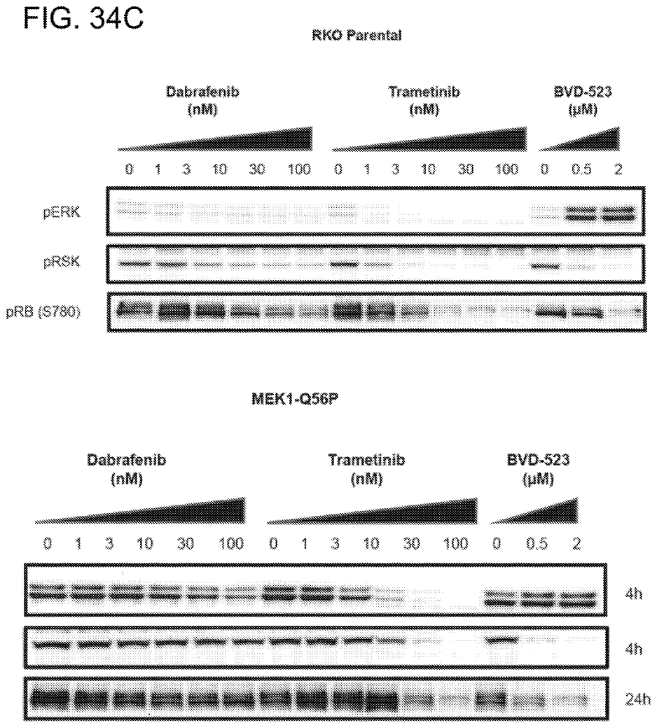

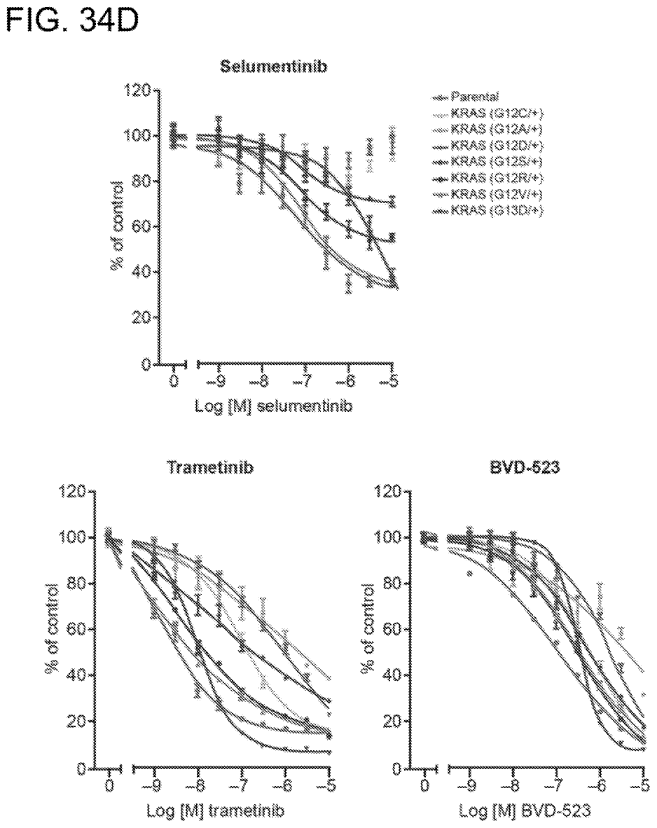

Emergence of resistance to BRAF and MEK inhibitors limits their clinical efficacy. Here, the experiments sought to model and compare the development of resistance to BRAF (dabrafenib), MEK (trametinib), and ERK1/2 (BVD-523) inhibition in vitro. Over several months, BRAF^{V600E}-mutant A375 cells were cultured in progressively increasing concentrations of each inhibitor. Drug-resistant A375 cell lines were readily obtained following growth in high concentrations of trametinib or dabrafenib, while developing cell lines with resistance to BVD-523 proved challenging (FIG. 34A). Overall, these in vitro data suggest that at concentrations yielding similar target inhibition, resistance to BVD-523 is delayed compared with dabrafenib or trametinib, and may translate to durable responses in the clinic.

Reactivation and dependence on ERK1/2 signaling is a common feature of acquired resistance to BRAF/MEK inhibition (Morris et al. 2013 and Hatzivassiliou et al. 2012); therefore, the activity of BVD-523 in in vitro models of acquired resistance was evaluated. First, a dabrafenib and trametinib combination-resistant A375 population was obtained using the increased concentration method described. The IC₅₀ and IC₅₀-fold change from parental A375 for dabrafenib, trametinib, and BVD-523 in the BRAF/MEK combination-resistant population is shown in Table 24. BVD-523 IC₅₀ was modestly shifted (2.5-fold), while dabrafenib and trametinib were more significantly shifted (8.5-fold and 13.5-fold, respectively) (Table 24). The cytotoxic agent paclitaxel was tested as a control with only a modest shift in potency observed. These data support the investigation of BVD-523 in the setting of BRAF/MEK therapy resistance, although the mechanism of resistance in this cell population remains to be characterized.

TABLE 24
BVD-523 activity in models
of BRAF/MEK inhibition

| Cell | | | BVD- | |
|---|---|---|---|---|
| Line | Dabrafenib | Trametinib | 523 | Paclitaxel |
| Parental | 2.1 | 0.2 | 129 | 1.9 |
| (IC₅₀ nM) | | | | |
| BRAFi- + | 17.9 | 2.7 | 323 | 3.5 |
| MEKi- | | | | |
| resistant | | | | |
| (IC₅₀ nM) | | | | |
| Fold | +8.5 | +13.5 | +2.5 | +1.8 |
| Change | | | | |

To further investigate the tractability of ERK1/2 inhibition in a model with a known mechanism of BRAF inhibitor resistance, AAV-mediated gene targeting was used to generate a pair of RKO BRAF^{V600E}-mutant cell lines isogenic for the presence or absence of an engineered heterozygous knock-in of MEK1^{Q56P}-activating mutation (Trunzer et al. 2013 and Emery et al. 2009). MEK1/2 mutations, including MEK1^{Q56P}, have been implicated in both single-agent BRAF and combination BRAF/MEK therapy-acquired resistance in patients (Wagle et al. 2011, Wagle et al. 2014, Emery et al. 2009 and Johnson et al. 2015). Single-agent assays demonstrated that relative to the parental BRAF^{V600E}::MEK1Wt cells, the double-mutant BRAF^{V600E}::MEK1^{Q56P} cells displayed a markedly reduced sensitivity to the BRAF inhibitors vemurafenib and dabrafenib and the MEK inhibitor trametinib (FIG. 34B). In contrast, response to BVD-523 was essentially identical in both the parental and MEK^{Q56P}-mutant cells, indicating that BVD-523 is not susceptible to this mechanism of acquired resistance. These results were confirmed in 2 independently derived double-mutant BRAF^{V600E}::MEK1^{Q56P} cell line clones, thus validating that results were specifically related to the presence of the MEK1^{Q56P} mutation rather than an unrelated clonal artifact (data not shown). Similar results were also observed with a second mechanistically distinct ERK1/2 inhibitor (SCH772984), supporting the expectation that these observations are specifically related to mechanistic inhibition of ERK1/2 and not due to an off-target compound effect.

To further characterize the mechanistic effects of BVD-523 on MAPK pathway signaling in BRAF^{V600E}::MEK1^{Q56P} cell lines, protein levels were assessed by Western blot (FIG. 34C). In the parental BRAF^{V600E} RKO cells, a reduced level of pRSK1/2 was observed following 4-hour treatment with BRAF (vemurafenib), MEK (trametinib), or ERK1/2 (BVD-523) inhibitors at pharmacologically active concentrations. In contrast, isogenic double-mutant BRAF^{V600E}.: MEK1^{Q56P} cells did not exhibit reduced RSK phosphorylation following BRAF or MEK inhibitor treatment, while BVD-523 remained effective in inhibiting pRSK1/2 to a level comparable to parental RKO. Similarly, pRB is reduced, indicating G0/G1 arrest, by 24 hours of BVD-523 treatment in both parental RKO and BRAF^{V600E}::MEK 1^{Q56P}.

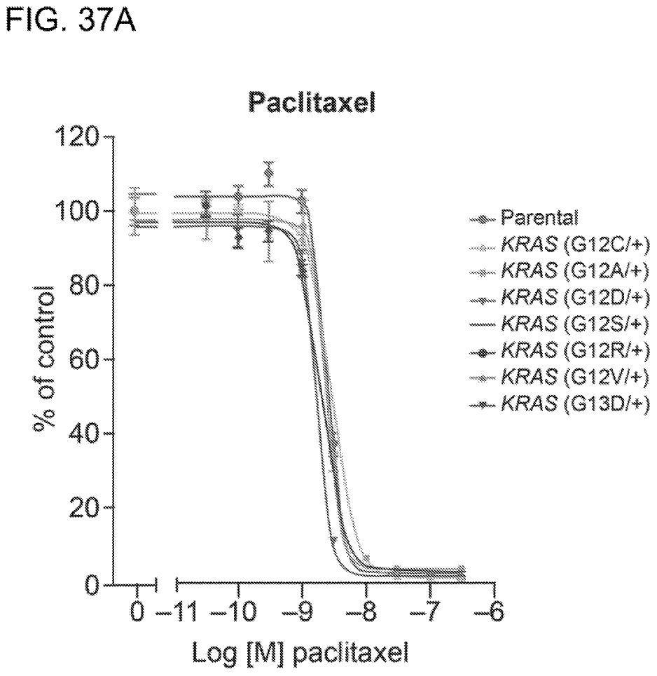

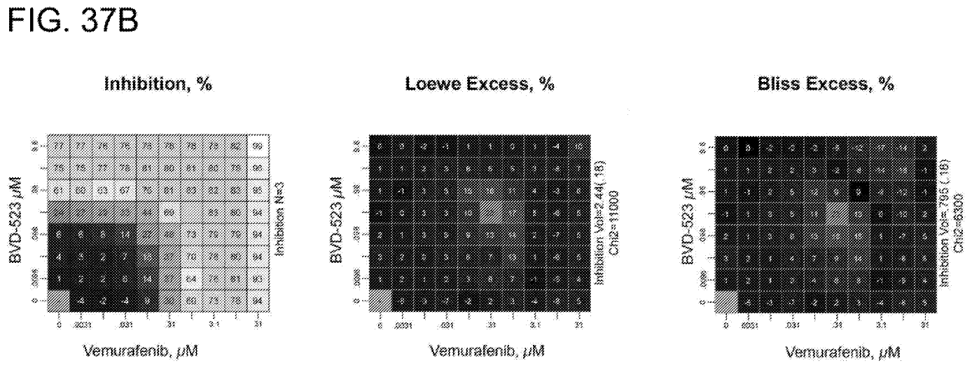

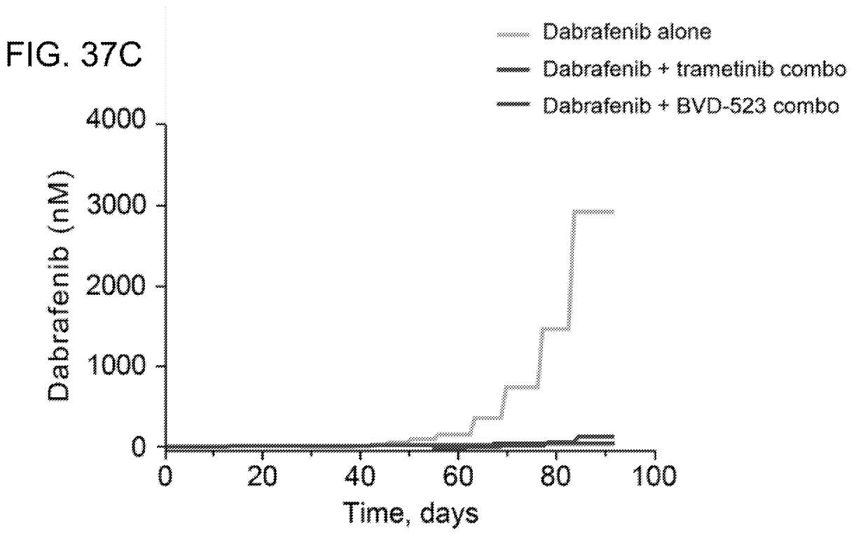

Acquired KRAS mutations are also known drivers of resistance to MAPK pathway inhibitors. To understand the susceptibility of BVD-523 to this mechanism of resistance, an isogenic panel of clinically relevant KRAS mutations in colorectal cell line SW48 was used. Sensitivity to BVD-523 was compared with MEK inhibitors selumetinib and trametinib (FIG. 34D). Sensitivity to paclitaxel was unaltered (FIG. 37A). While several mutant KRAS alleles conferred robust to intermediate levels of resistance to MEK inhibition, sensitivity to BVD-523 was unaltered by the majority of alleles, and where a shift in sensitivity was observed, it was not to the extent observed with trametinib or selumetinib. Overall, these data suggest that BVD-523 is more efficacious in this context than MEK inhibitors.

### Example 15

### BVD-523 Demonstrates In Vivo Activity in a BRAF Inhibitor-Resistant Patient-Derived Melanoma Xenograft Model

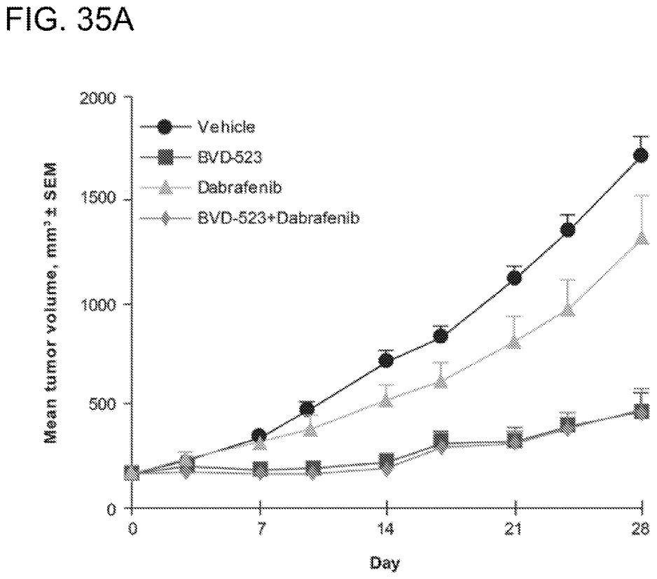

To confirm and extend the antitumor effects of BVD-523 observed in in vitro models of BRAF-/MEK-acquired resistance, a BRAF-resistant xenograft model derived from a patient with resistance to vemurafenib was utilized. BVD-523 was dosed by oral gavage at 100 mg/kg BID for 28 days, both alone and in combination with dabrafenib at 50 mg/kg BID (FIG. 35). As expected, minimal antitumor activity was demonstrated for single-agent dabrafenib (22% TGI). BVD-523 activity was significant compared with vehicle control (P≤0.05), with a TGI of 78%. In this model, combining BVD-523 with dabrafenib resulted in a TGI of 76% (P≤0.05); therefore, further benefit was not gained for the combination compared with single-agent BVD-523 in this model of BRAF-acquired resistance.

### Example 16

Combination Therapy with BVD-523 and a BRAF Inhibitor Provides Promising Antitumor Activity

Patients with BRAF-mutant cancer may acquire resistance to combined BRAF/MEK therapy (Wagle et al. 2014), warranting consideration of other combination approaches within the MAPK pathway. The anti-proliferative effects of combining BVD-523 with the BRAF inhibitor vemurafenib was assessed in the BRAF^{V600E}-mutant melanoma cell line G-361. As anticipated, single agents BVD-523 and vemurafenib were both active, and modest synergy was observed when combined (FIG. 37B). This indicates that BVD-523 combined with BRAF inhibitors are at least additive and potentially synergistic in melanoma cell lines carrying a BRAF^{V600E} mutation. Furthermore, generating acquired resistance in vitro following continuous culturing of BRAF^{V600E} mutant cell line (A375) in BRAF inhibitor plus BVD-523 was challenging. In contrast generating resistance to dabrafenib alone occurred relatively rapidly (FIG. 37C). Even resistance to combined dabrafenib and trametinib emerged before dabrafenib plus trametinib.

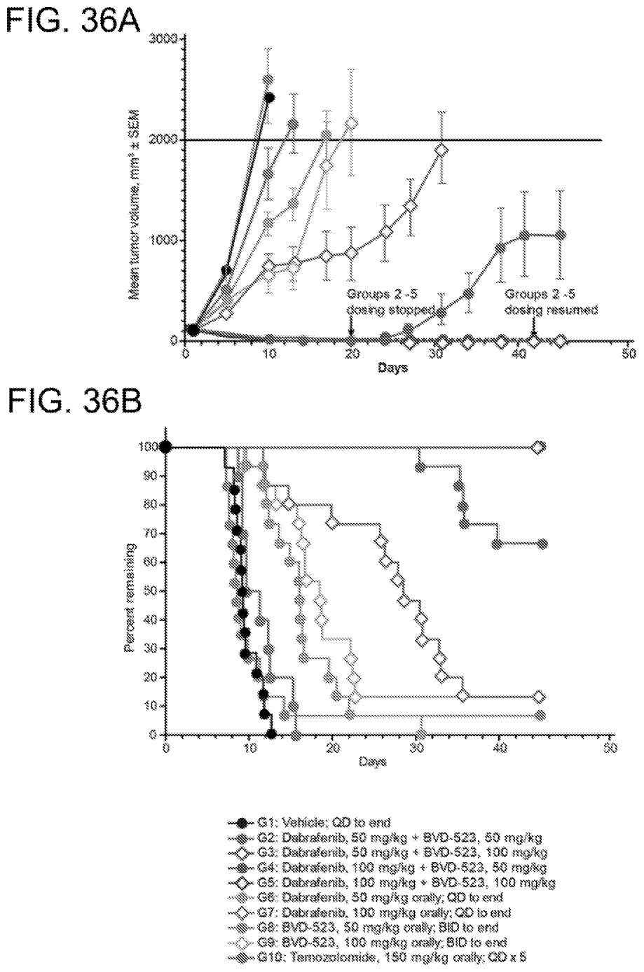

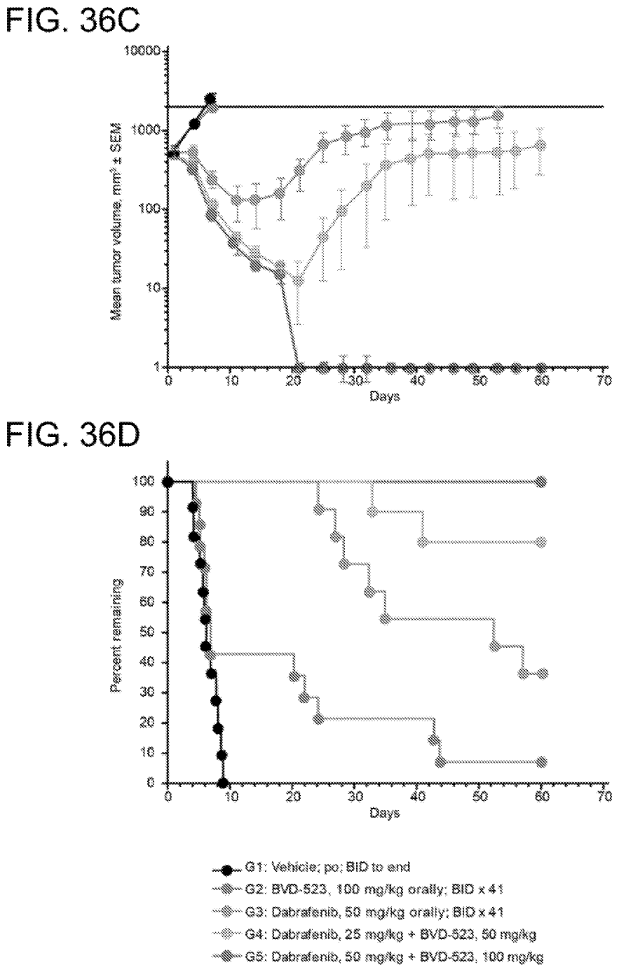

The benefit of combined BRAF and ERK inhibition may not be fully realized in in vitro combination studies where concentrations are not limited by tolerability. To understand the benefit of the combination, efficacy was assessed in vivo utilizing xenografts of the BRAF^{V600E}-mutant human melanoma cell line A375. Due to the noteworthy response to combination treatment, dosing in the combination groups was stopped on Day 20 to monitor for tumor regrowth, and was reinitiated on Day 42 (FIG. 36A). Tumors were measured twice weekly until the study was terminated on Day 45. The median time to endpoint (TTE) for controls was 9.2 days, and the maximum possible tumor growth delay (TGD) of 35.8 days was defined as 100%. Temozolomide treatment resulted in a TGD of 1.3 days (4%) and no regressions. The 50- and 100-mg/kg dabrafenib monotherapies produced TGDs of 6.9 days (19%) and 19.3 days (54%), respectively, a significant survival benefit (P<0.001), and 1 PR in the 100-mg/kg group. The 100-mg/kg BVD-523 monotherapy resulted in a TGD of 9.3 days (26%), a significant survival benefit (P<0.001), and 2 durable complete responses. The combinations of dabrafenib with BVD-523 each produced the maximum possible 100% TGD with noteworthy regression responses, and statistically superior overall survival compared with their corresponding monotherapies (P<0.001). The lowest dose combination produced a noteworthy 7/15 tumor-free survivors (TFS), and the 3 higher-dosage combinations produced a total of 43/44 TFS, consistent with curative or near-curative activity (FIG. 36B). In summary, the combination of dabrafenib with BVD-523 produced a greater number of TFS and superior efficacy to either single agent.

Based on the activity of BVD-523 plus dabrafenib in A375 xenograft models with a starting tumor volume of approximately 75-144 mm³, a follow-up experiment was conducted to determine the efficacy of combination therapy in “upstaged” A375 xenografts (average tumor start volume, 700-800 mm³) (FIG. 36C). The median TTE for controls was 6.2 days, establishing a maximum possible TGD of 53.8 days, which was defined as 100% TGD for the 60-day study. BVD-523 100-mg/kg monotherapy produced a negligible TGD (0.7 day, 1%) and no significant survival difference from controls (P>0.05). The distribution of TTEs and 2 PRs suggested there may have been a subset of responders to treatment with BVD-523 alone. Dabrafenib 50-mg/kg monotherapy was efficacious, yielding a TGD of 46.2 days (86%) and a significant survival benefit compared with controls (P<0.001). This group had 5 PRs and 5 CRs, including 3 TFS, among the 11 evaluable mice (FIG. 36D). Both combinations of dabrafenib with BVD-523 produced the maximum 100% TGD and a significant survival benefit compared with controls (P<0.001). Each combination produced 100% regression responses among evaluable mice, though there were distinctions in regression activity. The 25-mg/kg dabrafenib and 50-mg/kg BVD-523 combination had 2 PRs and 8 CRs, with 6/10 TFS, whereas the 50-mg/kg dabrafenib and 100-mg/kg BVD-523 combination had 11/11 TFS on Day 60 (FIG. 36D). Overall, these data support the rationale for frontline combination of BVD-523 with BRAF-targeted therapy in BRAF^{V600}-mutant melanoma, and this is likely to extend to other tumor types harboring this alteration.

### Discussion

BVD-523 is a potent, highly selective, reversible, small molecule ATP-competitive inhibitor of ERK1/2 with activity in in vivo and in vitro cancer models. In vitro, BVD-523 demonstrated potent inhibition against several human tumor cell lines, particularly those harboring activating mutations in the MAPK signaling pathway, consistent with its mechanism of action. BVD-523 elicited changes in downstream target and effector proteins, including inhibition of direct substrate of ERK1/2, pRSK, and total DUSP6 protein levels. These findings are in line with those of previous studies of other ERK1/2 inhibitors, which demonstrated effective suppression of pRSK with ERK1/2 inhibition (Morris et al. 2013 and Hatzivassiliou et al. 2012). Interestingly, BVD-523 treatment resulted in a marked increase in ERK1/2 phosphorylation in vitro and in vivo. Similar to our findings, an increase in pERK1/2 has been reported with the ERK1/2 inhibitor Vx11e; conversely, pERK1/2 inhibition occurs with SCH772984 (Morris et al. 2013). Although differences in pERK1/2 levels were observed among the various ERK1/2 inhibitors tested, downstream effectors (i.e., pRSK1 and total DUSP6) were similarly inhibited. These findings suggest quantifying ERK1/2 target substrates, such as pRSK1, may serve as reliable pharmacodynamic biomarkers for BVD-523-mediated inhibition of ERK1/2 activity.

While BRAF (dabrafenib, vemurafenib) and MEK (trametinib, cobimetinib) inhibitors validate the MAPK pathway as a therapeutic target, particularly in patients with BRAF^{V600} mutations, the antitumor response is limited by the emergence of acquired resistance and subsequent disease progression. Resistance has been attributed to the upregulation and activation of compensatory signaling molecules (Nazarian et al. 2010, Villanueva et al. 2010, Johannessen et al. 2010 and Wang et al. 2011), amplification of the target genes (Corcoran et al. 2010), and activating mutations of pathway components (e.g., RAS, MEK) (Wagle et al. 2011, Emery et al. 2009 and Wang et al. 2011). Reactivation of the ERK1/2 pathway is one common consequence of acquired resistance mechanism. When introduced into the BRAF^{V600E}-mutant melanoma cell line A375, MEK^{Q56P} conferred resistance to MEK and BRAF inhibition (Wagle et al. 2011). By contrast, BVD-523 retained its potent inhibitory activity in the engineered MEK^{Q56P} cell line, indicating that ERK1/2 inhibition is effective in the setting of upstream activating alterations which can arise in response to BRAF/MEK treatment. As further evidence of a role for BVD-523 in the context of acquired resistance, efficacy of BVD-523 was evident in a xenograft model derived from a tumor sample from a patient whose disease progressed on vemurafenib; the BRAF inhibitor dabrafenib was not effective in this model. These data support a role for targeting ERK1/2 in the setting of BRAF/MEK resistance, and complement previously published findings (Morris et al. 2013 and Hatzivassiliou et al. 2012). To further characterize resistance to inhibitors of the MAPK pathway, the emergence of resistance to BVD-523 itself was investigated. It was found that single-agent treatment of cancer cells with BVD-523 was durable and more challenging to develop resistance compared with other agents targeting upstream MAPK signaling components (i.e., dabrafenib, trametinib). This may suggest that acquiring resistance to ERK1/2-targeting agents is harder to achieve than acquiring resistance to BRAF or MEK therapy, potentially due to the fact that BVD-523 preferentially targets the more conserved active confirmation of the ATP binding site. However, in vitro studies with other ERK1/2 inhibitors have identified specific mutants in ERK1/2 that drive resistance (Jha et al. 2016 and Goetz et al. 2014); these specific mutations have yet to be identified in clinical samples from ERK1/2 inhibitor-relapsed patients.

The potential clinical benefit of ERK1/2 inhibition with BVD-523 extends beyond the setting of BRAF/MEK therapy-resistant patients. As ERK1/2 is a downstream master node within this MAPK pathway, its inhibition is attractive in numerous cancer settings where tumor growth depends on MAPK signaling. Approximately 30% of all cancers harbor RAS mutations; therefore, targeting downstream ERK1/2 with BVD-523 is a rational treatment approach for these cancers. Furthermore, results from a study by Hayes et al. indicate that prolonged ERK1/2 inhibition in KRAS-mutant pancreatic cancer is associated with senescent-like growth suppression (Hayes et al. 2016). However, a combination approach may be required for maximal and durable attenuation of MAPK signaling in the setting of RAS mutations. For example, MEK inhibition in KRAS-mutant colorectal cancer cell results in an adaptive response of ErbB family activation, which dampens the response to MEK inhibition (Sun et al. 2014). Similar context-specific adaptive responses may occur following ERK1/2 inhibition with BVD-523. The optimal treatment combinations for various genetic profiles and cancer histologies are the subject of ongoing research. In addition to BRAF^{V600} and RAS mutations, other alterations which drive MAPK are emerging. For example, novel RAF fusions and atypical non-V600 BRAF mutations which promote RAF dimerization activate the MAPK pathway (Yao et al. 2015). BRAF inhibitors such as vemurafenib and dabrafenib which inhibit BRAF^{V600}-mutant monomer proteins have been shown to be inactive in atypical RAF alterations which drive MAPK signaling in a dimerization-dependent manner (Yao et al. 2015). However, treatment with BVD-523 to target downstream ERK1/2 in these tumors may be a novel approach to addressing this unmet medical need.

In the setting of BRAF^{V600}-mutant melanoma tumors, combined BRAF and MEK inhibition exemplifies how agents targeting different nodes of the same pathway can improve treatment response and duration. Our combination studies in BRAF^{V600E}-mutant xenografts of human melanoma cell line A375 provides support for combination therapy with BVD-523 and BRAF inhibitors. The combination demonstrated superior benefit relative to single-agent treatments, including results consistent with curative responses. The clinical efficacy and tolerability of combined BRAF/BVD-523 therapy remains to be determined. It would not be unreasonable to expect that a BRAF/ERK1/2 combination will at least be comparable in efficacy to a targeted BRAF/MEK combination. Furthermore, the in vitro observation that acquired resistance to BVD-523 is more challenging to achieve compared with other MAPK pathway inhibitors suggests that the BRAF/BVD-523 inhibitor combination has the potential to provide a more durable response.

Significant progress has also been made using immunotherapy for melanoma. The US FDA has approved various immune checkpoint inhibitors for the treatment of advanced melanoma, including the cytotoxic T-lymphocyte antigen-4 targeted agent ipilimumab and the programmed death-1 inhibitors pembrolizumab and nivolumab. Combining BVD-523 with such immunotherapies is an attractive therapeutic option; further investigation is warranted to explore dosing schedules and to assess whether synergistic response can be achieved.

Based on the preclinical data, BVD-523 may hold promise for treatment of patients with malignancies dependent on MAPK signaling, including those whose tumors have acquired resistance to other treatments. The clinical development of BVD-523 is described below. See, Examples 17-24

### Example 17

Phase I Dose-Escalation Study of the First-In-Class Novel Oral ERK1/2 Kinase Inhibitor BVD-523 (Ulixertinib) in Patients with Advanced Solid Tumors

The present invention describes the first-in-human dose escalation study of an ERK1/2 inhibitor for the treatment of patients with advanced solid tumors. BVD-523 has an acceptable safety profile with favorable pharmacokinetics and early evidence of clinical activity.

Mitogen-activated protein kinase (MAPK) signaling via the RAS-RAF-MEK-ERK cascade plays a critical role in oncogenesis; thus attracting significant interest as a therapeutic target. This ubiquitous pathway is composed of RAS upstream of a cascade of the protein kinases RAF, MEK1/2, and ERK1/2. RAS is activated by GTP binding, which in turn results in activation of each protein kinase sequentially. Although they appear to be the only physiologic substrates for MEK1/2, ERK1/2 have many targets in the cytoplasm and nucleus, including the transcription factors Elk1, c-Fos, p53, Ets1/2, and c-Jun (Shaul et al. 2007). ERK1/2 activation and kinase activity influences cellular proliferation, differentiation, and survival through a variety of mechanisms (Rasola et al. 2010), including activation of the ribosomal S6 kinase (RSK) family members (Romeo et al. 2012).

Constitutive, aberrant activation of the RAS-RAF-MEK1/2-ERK1/2 signaling pathway has been identified and implicated in the development or maintenance of many cancers (Schubbert et al. 2007 and Gollob et al. 2006). Mutations in RAS family genes, such as KRAS, NRAS, and HRAS are the most common, with activating RAS mutations occurring in ~30% of human cancers (Schubbert et al. 2007). KRAS mutations are prevalent in pancreatic (>90%) (Kanda et al. 2012), biliary tract (3%-50%) (Hezel et al. 2014), colorectal (30%-50%) (Arrington et al. 2012), lung (27%) (Pennycuick et al. 2012), ovarian (15%-39%) (Dobrzycka et al. 2009), and endometrioid endometrial (18%) (O'Hara and Bell 2012) cancers; NRAS mutations are prevalent in melanoma (20%) (Khattak et al. 2013) and myeloid leukemia (8%-13%) (Yohe 2015); and HRAS mutations are prevalent in bladder (12%) cancer (Fernández-Medarde and Santos 2011). Mutations in RAF family genes, most notably BRAF, are frequent, particularly in melanoma. BRAF mutations have been identified in 66% of malignant melanomas and in ~7% of a wide range of other cancers (Davies et al. 2002), while MEK mutations are rarer, occurring at an overall frequency of 8% in melanomas (Nikolaev et al. 2012). In contrast, ERK mutations resulting in tumorigenesis have been reported only rarely to date (Deschenes-Simard et al. 2014).

The US Food and Drug Administration (FDA) has approved two selective BRAF inhibitors, vemurafenib and dabrafenib, as monotherapies for patients with BRAF^{V600}-mutant metastatic melanoma (Taflinar [package insert] and Zelboraf [package insert]). Though response rates for these targeted therapies can be as high as 50% in in patients with BRAF^{V600} mutations, duration of response is often measured in months, not years (Hauschild et al. 2012 and McArthur et al. 2014). The MEK1/2 inhibitor trametinib is also approved as a monotherapy in this setting (Mekinist [package insert]), but is more commonly used in combination with the BRAF inhibitor dabrafenib. First-line use of trametinib administered in combination with dabrafenib offers an even greater improvement in overall survival compared with vemurafenib monotherapy without increased overall toxicity (Robert et al. 2015), highlighting the potential utility of simultaneously targeting multiple proteins of this MAPK signaling pathway. This therapeutic combination was also associated with a lower incidence of MEK inhibitor-associated rash and BRAF inhibitor-induced hyperproliferative skin lesions compared with each single agent alone (Flaherty et al. 2012). Recently, a phase III trial also demonstrated significant improvements in overall survival (25.1 vs. 18.7 months, hazard ratio [HR] 0.71, P=0.0107), progression-free survival (PFS) (11.0 vs. 8.8 months, HR 0.67, P=0.0004), and overall response (69% vs. 53%; P=0.0014) with dabrafenib plus trametinib versus dabrafenib alone in patients with BRAF^{V600E}/K mutation-positive melanoma (Long et al. 2015). Similarly, significant improvements in PFS (9.9 vs. 6.2 months, HR 0.51, P<0.001) and the rate of complete response (CR) or partial response (PR) (68% vs. 45%; P<0.001) have been demonstrated with the combination of cobimetinib plus vemurafenib compared with vemurafenib alone (Larkin et al. 2014). To this end, FDA approval was recently granted for the combination of vemurafenib and cobemetinib for BRAF^{V600E}/K-mutated melanoma. Based on these and related findings, the combination of a BRAF inhibitor plus a MEK inhibitor has become a standard targeted treatment option for patients with metastatic melanoma containing BRAF^{V600E}/K mutations.

Though BRAF/MEK-targeted combination therapy has been demonstrated to provide significant additional benefit beyond single-agent options, most patients eventually develop resistance and disease progression after ~12 months (Robert et al. 2015, Flaherty et al. 2012 and Long et al. 2015). Several mechanisms of acquired resistance following either single-agent or combination therapies have been identified, including the generation of BRAF splicing variants, BRAF amplification, development of NRAS or MEK mutations, and upregulation of bypass pathways (Poulikakos et al. 2011, Corcoran et al. 2010, Nazarian et al. 2010, Shi et al. 2014, Johannessen et al. 2010, Wagle et al. 2011, Wagle et al. 2014 and Ahronian et al. 2015). Central to many of these mechanisms of resistance is the reactivation of ERK signaling, which enables the rapid recovery of MAPK pathway signaling and escape of tumor cells from single-agent BRAF or combination BRAF/MEK inhibitor therapies (Paraiso et al. 2010). ERK inhibition may provide the opportunity to avoid or overcome resistance from upstream mechanisms, as it is the most distal master kinase of this MAPK signaling pathway. This is supported by preclinical evidence that inhibition of ERK by small molecule inhibitors acted to both inhibit the emergence of resistance and overcome acquired resistance to BRAF and MEK inhibitors (Morris et al. 2013 and Hatzivassiliou et al. 2012).

BVD-523 is a highly potent, selective, reversible, ATP-competitive ERK1/2 inhibitor which has been shown to reduce tumor growth and induce tumor regression in BRAF and RAS mutant xenograft models. Furthermore, single-agent BVD-523 inhibited human xenograft models that were cross-resistant to both BRAF and MEK inhibitors. See, Examples 9-16. Therefore, an open-label, first-in-human study (Clinicaltrials.gov identifier, NCT01781429) of oral BVD-523 to identify both the maximum tolerated dose and the recommended dose for further study was undertaken. The present study also aimed to assess pharmacokinetic and pharmacodynamic properties as well as preliminary efficacy in patients with advanced cancers.

### Example 18

### Patient Characteristics

A total of 27 patients were enrolled and received at least one dose of study drug from Apr. 4, 2013 to Dec. 1, 2015. Baseline demographics and disease characteristics are shown in Table 25. The median patient age was 61 years (range, 33-86 years). Fifty-two percent ( 14/27) of patients were male and 63% ( 17/27) had an Eastern Cooperative Oncology Group (ECOG) performance status of 1. Melanoma was the most common cancer (30%; BRAF mutation present in ⅞ of these patients). The remaining patients had colorectal (19%; 5/27), papillary thyroid (15%; 4/27), or non-small cell lung cancer (NSCLC) (7%; 2/27), and 8 (30%) were classified as having other cancers (2 pancreatic, 1 appendiceal, 1 nonseminomatous germ cell, 1 ovarian and 3 with unknown primary). The majority of patients had received 2 or more prior lines of systemic therapy, with 41% ( 11/27) receiving 2 to 3 and 48% ( 13/27) receiving >3 prior lines of systemic therapy.

TABLE 25
Baseline demographics and clinical
characteristics of patients

| Parameter | N = 27 |
|---|---|
| Median age, years (range) | 61 (33-86) |
| Sex, n (%) | |
| Female | 13 (48) |
| Male | 14 (52) |
| Ethnicity, n (%) | |
| Not Hispanic/Latino | 27 (100) |
| ECOG performance status | |
| 0 | 10 (37) |
| 1 | 17 (63) |
| Cancer type, n (%) | |
| Melanoma^{a} | 8 (30) |
| Colorectal | 5 (19) |
| Papillary thyroid | 4 (15) |
| Non-small cell lung | 2 (7) |
| Other^{b} | 8 (30) |
| Molecular abnormalities, n (%)^{c} | |
| BRAF mutant | 13 (48) |
| KRAS mutant | 6 (22) |
| NRAS mutant | 2 (7) |
| Other^{d} | 7 (26) |
| Unknown | 4 (15) |
| Number of prior systemic | |
| anticancer regimens, | |
| n (%) | 1 (4) |
| 0 | 2 (7) |
| 1 | 11 (41) |
| 2-3 | 13 (48) |
| >3 | |
| Prior BRAF/MEK-targeted | 11 (41) |
| therapy^{e}, n (%) | |
| BRAF | 5 (19) |
| MEK | 6 (22) |
| BRAF/MEK | 2 (7) |

^{a}Seven were BRAF mutant and 1 was unknown.
^{b}Two pancreatic, 1 appendiceal, 1 non-seminomatous germ cell, 1 ovarian, 3 unknown primary.
^{c}Patients may have more than 1 molecular abnormality.
^{d}Other molecular abnormalities included ERCC1, RRM1, thymidylate synthetase, GNAS, MEK1, TP53, CREBBP, ROS1, PTEN, AKT3, and PIK3CA.
^{e}Some patients were treated with more than one BRAF inhibitor.
Abbreviation: ECOG, Eastern Cooperative Oncology Group.

### Example 19

### Ex Vivo Effects of BVD-523 on RSK1/2 Phosphorylation

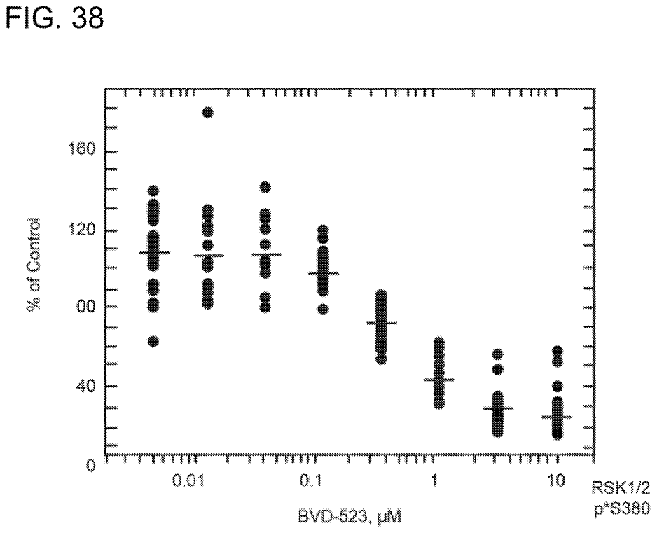

An ex vivo biomarker assay that could be used to support clinical studies was developed to demonstrate the inhibitory effects of BVD-523 on ERK activity. The assay extends preclinical cellular data where inhibitors of MAPK signaling, such as BVD-523, dabrafenib, trametinib, and vemurafenib, have been shown to inhibit RSK phosphorylation as a function of inhibitor concentration in BRAF mutant cancer cell lines. See, Examples 9-16. Specifically, ERK inhibitor-dependent inhibition of phorbol 12-myristate 13-acetate (PMA)-stimulated phosphorylation of the ERK substrate RSK1 in whole blood was used as a target marker. When BVD-523 was added directly to whole blood from healthy volunteers, PMA-stimulated RSK phosphorylation decreased with increasing concentrations of BVD-523 (FIG. 38). The mean IC₅₀ for the cumulative data was 461±20 nM for BVD-523, with a maximum inhibition of 75.8±2.7% at 10 UM BVD-523. Maximum inhibition was defined as the RSK phosphorylation measured in the presence of 10 M BVD-523. Patient-derived whole blood samples, collected just prior to dosing or at defined timepoints following dosing with BVD-523, were similarly treated and RSK phosphorylation levels quantitated.

### Example 20

### Dose Escalation, Dose-Limiting Toxicities (DLTs), Maximum Tolerated Dose (MTD), and Recommended Phase II Dose (RP2D)

As per protocol, 5 single-patient cohorts (from 10 to 150 mg twice-daily [BID]) proceeded without evidence of a DLT. The 300-mg BID cohort was expanded to more fully characterize BVD-523 exposures. One of 6 patients given 600 mg BID experienced a DLT of Grade 3 rash. The 900-mg BID dose exceeded the MTD, with one patient experiencing Grade 3 pruritus and elevated aspartate aminotransferase (AST) and another patient experiencing Grade 3 diarrhea, vomiting, dehydration, and elevated creatinine (Table 26). The subsequent intermediate dose of 750 mg BID also exceeded the MTD, with DLTs of Grade 3 rash and Grade 2 diarrhea in 1 patient and Grade 2 hypotension, elevated creatinine, and anemia in another patient. Therefore, the MTD and RP2D were determined to be 600 mg BID.

TABLE 26
Dose-limiting toxicities in Cycle 1 (21 days)

| Dose, | | |
|---|---|---|
| mg | DLT | |
| (BID) | Frequency | DLT Description |
| 10 | 0/1 | N/A |
| 20 | 0/1 | N/A |
| 40 | 0/1 | N/A |
| 75 | 0/1 | N/A |
| 150 | 0/1 | N/A |
| 300 | 0/4 | N/A |
| 600 | 1/8 | Rash (Grade 3) |
| 750^{a} | 2/4 | Rash (Grade 3), diarrhea (Grade 2) |
| | | Hypotension (Grade 2), elevated creatinine (Grade 2), |
| | | anemia (Grade 2), delay to cycle 2 dosing |
| 900 | 2/7 | Pruritus (Grade 3), elevated AST (Grade 3) |
| | | Diarrhea (Grade 3), vomiting (Grade 3), dehydration |
| | | (Grade 3), elevated creatinine (Grade 3) |

^{a}Intermediate dose.
Abbreviations: AST, aspartate transaminase, BID, twice daily; DLT, dose-limiting toxicity; N/A, not applicable.

### Example 21

### Adverse Events (AEs)

Investigator-assessed treatment-related AEs of any grade were noted in 26 of 27 patients (96%). The most common treatment-related AEs (>30%) were rash (predominately acneiform) (70%), fatigue (59%), diarrhea (52%), and nausea (52%) (Table 27). No patients experienced a Grade 4 or 5 treatment-related AE or discontinued treatment due to a treatment-related AE. Most events were Grade 1 to 2, with treatment-related Grade 3 events noted in 13 of 27 patients (48%). The only Grade 3 treatment-related events present in ≥10% of patients were diarrhea (15%) and increased liver function tests (11%), all of which occurred above the 600-mg BID dose.

TABLE 27
Adverse events possibly/definitely related to
BVD-523 in ≥10% of patients

| | N = 27 | | |
|---|---|---|---|
| | | Grade | Grade |
| | Any grade, | 1 or 2, | 3^{a}, n |
| Event | n (%) | n (%) | n (%) |
| Rash | 20 (74) | 18 (67) | 2^{b} (7) |
| Fatigue | 17 (63) | 16 (59) | 1 (4) |
| Diarrhea | 16 (59) | 12 (44) | 4 (15) |
| Nausea | 14 (52) | 14 (52) | 0 |
| Vomiting | 8 (30) | 7 (26) | 1 (4) |
| Anorexia | 6 (22) | 6 (22) | 0 |
| Pruritus | 6 (22) | 6 (22) | 0 |
| Anemia | 5 (19) | 3 (11) | 2 (7) |
| Increased | 5 (19) | 4 (15) | 1 (4) |
| creatinine | | | |
| Dehydration | 5 (19) | 3 (11) | 2 (7) |
| Peripheral edema | 4 (15) | 4 (15) | 0 |
| Increased LFTs | 4 (14) | 1 (4) | 3 (11) |
| (ALT and AST) | | | |
| Blurry/dimmed | 3 (11) | 3 (11) | 0 |
| visionb | | | |
| Constipation | 3 (11) | 3 (11) | 0 |
| Fever | 3 (11) | 3 (11) | 0 |

^{a}No patients experienced Grade 4 or 5 AEs that were possibly or definitely related to BVD-523 treatment.
^{b}Acneiform and maculo-papular rash.
^{c}One Grade 1 event of related central serous retinopathy.
Analysis cut-off date: Dec. 1, 2015.
Abbreviations: AEs, adverse events; ALT, alanine transaminase, AST, aspartate transaminase, LFTs, liver function tests.

Fourteen patients experienced a total of 28 serious AEs (SAEs). Nine of these were considered to be related or possibly related to BVD-523 by the investigator, which included dehydration, diarrhea, or elevated creatinine (2 patients each), vomiting, nausea, and fever (1 patient each). All other SAEs were considered to be unrelated to treatment with BVD-523. Dose reductions resulting from AEs occurred in 3 patients during the study: 1 patient reduced from 600 mg BID to 300 mg BID and 2 patients reduced from 900 mg BID to 600 mg BID.

### Example 22

### Pharmacokinetics

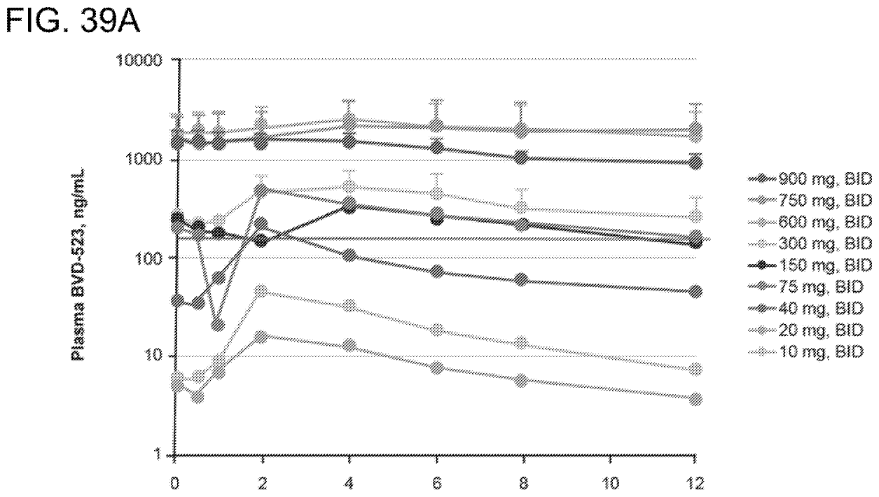

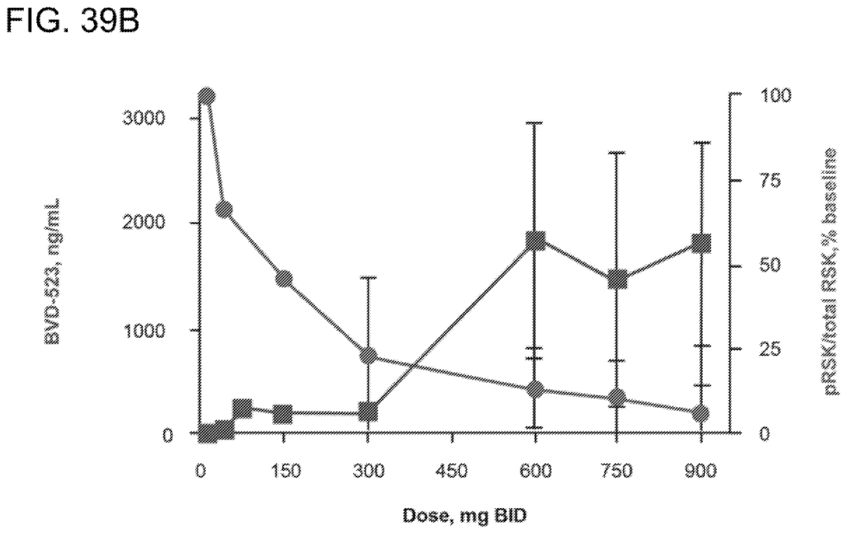

Single-dose and steady-state pharmacokinetics of BVD-523 are summarized in FIG. 39A and Table 28. Generally, orally administered BVD-523 was slowly absorbed in patients with advanced malignancies. After reaching the maximum concentration (Cₘₐₓ), plasma BVD-523 levels remained sustained for approximately 2 to 4 hours. Subsequently, plasma drug concentrations slowly declined. Since plasma drug concentrations were measured only up to 12 hours after the morning dose, it was not possible to calculate an effective or terminal phase elimination rate. BVD-523 pharmacokinetics were linear and dose proportional in terms of both Cₘₐₓ and area under the curve (AUC) when administered up to 600 mg BID. A further increase in exposure was not observed as the dose increased from 600 to 900 mg BID. The Cₘₐₓ reached the level of the EC₅₀ based on the ex vivo whole blood assay (~200 ng/ml) for all doses above 20 mg BID. Additionally, steady-state exposures remained at or above the target EC₅₀ for dose levels of ≥150 mg BID throughout the dosing period. Minimal plasma accumulation of BVD-523 and its metabolites were observed on Day 15 at the lower (<75 mg BID) dose levels, whereas accumulation ranged from approximately 1.3- to 4.0-fold for the higher dose levels. Predose concentrations on Day 22 were generally similar to those on Day 15, indicating that steady state had already been attained by Day 15 (data not shown). The degree of interpatient variability in plasma exposure to BVD-523 and its metabolites was considered moderate and not problematic.

TABLE 28
Steady-state BVD-523 pharmacokinetics (Cycle 1, Day 15)

| | | C_{max ′} ng/mL ± SD | | AUC_{0-12 ′} ng · hr/mL ± SD | |
|---|---|---|---|---|---|
| Dose, mg^{a} | n = | Day 1 | Day 15 | Day 1 | Day 15 |
| 10 | 1 | 48.2 | 45.7 | 220 | 234 |
| 20 | 1 | 14.9 | 15.8 | 91.7 | 98.7 |
| 40 | 1 | 100 | 191 | 614 | 999 |
| 150 | 1 | 133 | 326 | 817 | 2770 |
| 300 | 4^{b} | 765 ± 234 | 586 ± 257 | 4110 ± 1140 | 4460 ± 2460 |
| 600^{c} | 7^{d} | 1110 ± 589 | 2750 ± 1740 | 2750 ± 1740 | 24400 ± 16200 |
| 750 | 4^{h} | 1450 ± 539 | 2290 ± 1790^{f} | 10700 ± 1120^{g} | 23300 ± 19800^{f} |
| 950 | 7^{e} | 1430 ± 1010 | 1720 ± 328 | 10800 ± 6320^{h} | 15900 ± 1300^{e} |

^{a}Dose level administered twice daily;
^{b}n = 3 on Day 15;
^{c}Number of subjects for Day 15 at the 600 mg dose level includes two subjects who started Day 1 dosing at 900 mg and were later reduced to 600 mg;
^{d}n = 8 on Day 15;
^{e}n = 4 on Day 15;
^{f}One subject started on Day 1 dosing at 750 mg and was later reduced to 450 mg, Day 15 parameters for this subject reflect at least 10 consecutive doses at 450 mg/dose. Individual Day 15 parameters were 1300 ng/mL for Cₘₐₓ and 10700 ng·hr/mL for AU_{C0-12};
^{g}n = 3;
^{h}n =5.

The urinary excretion after first dose and at steady state of BVD-523 was negligible (<0.2% of the dose) at all dose levels within 12 hours postdose, and not dose-related within this very low percentage range. Renal clearance appeared to be dose-independent. Individual renal clearance values ranged from 0.128 to 0.0895 L/hr (where n=1 per dose level) and mean values ranged from 0.0149 to 0.0300 L/hr (where n≥3).

### Example 23

### Pharmacodynamic Confirmation of Target Inhibition by BVD-523

To confirm on-target and pathway inhibition by BVD-523, RSK-1 phosphorylation was examined as a target biomarker in human whole blood samples from patients with solid tumors who received BVD-523. Steady state whole blood samples collected just prior to Day 15 dosing from BVD-523-treated patients displayed concentration-dependent inhibition of PMA stimulated ERK activity (FIG. 39B), ranging from 0% ERK inhibition with BVD-523 dosing at 10 mg BID to 93±8% ERK inhibition with dosing at 900 mg BID. The plasma concentrations of BVD-523 that yielded 50% inhibition of RSK phosphorylation were similar whether BVD-523 was spiked directly into healthy volunteer plasma or was present following oral dosing of patients.

### Example 24

### Antitumor Effects

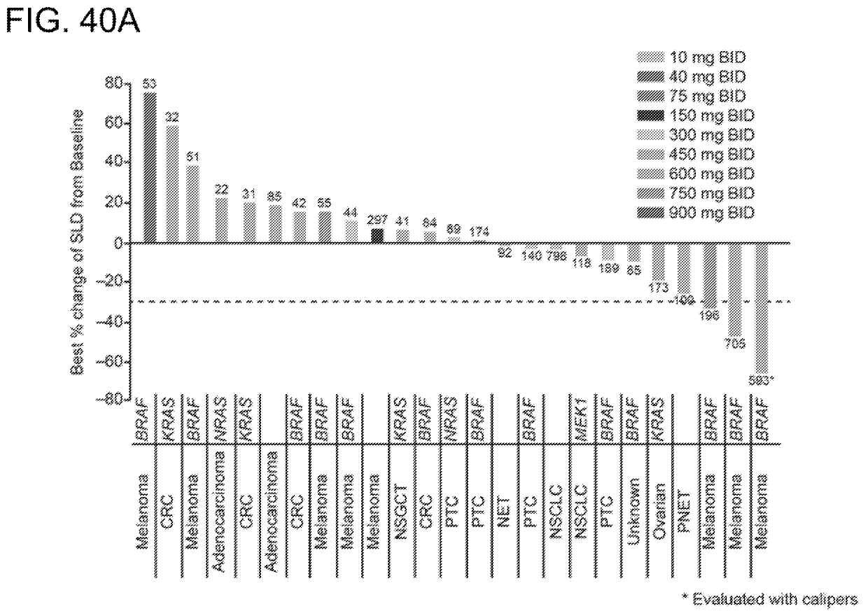

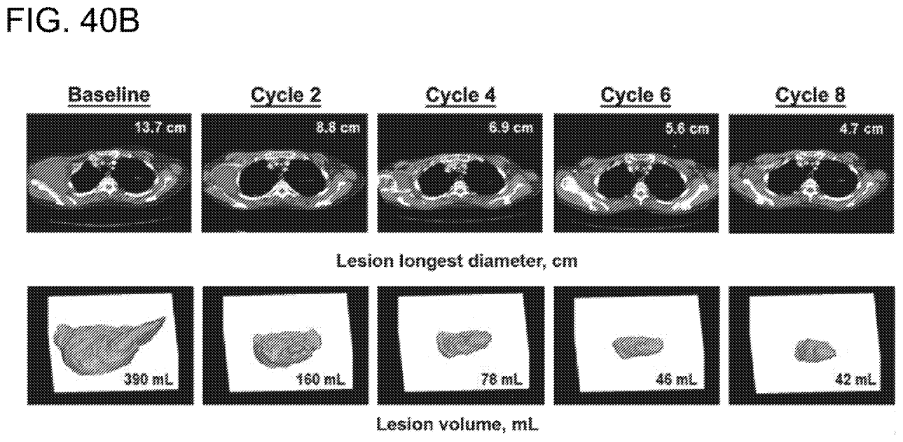

Tumor response to BVD-523 was assessed in 25 evaluable patients using Response Evaluation Criteria in Solid Tumors version 1.1 (RECIST v1.1); 2 patients did not receive both scans of target lesions and were thus not evaluated using RECIST v1.1. No patients achieved a complete response, but 3 patients (all patients with melanoma with BRAF^{V600} mutations) achieved a partial response (129 days [BRAF/MEK-inhibitor naïve], 294 days ongoing at [refractory to prior BRAF/MEK inhibitors], 313 days ongoing by the data cutoff date [intolerant to other BRAF/MEK inhibitors]) (FIG. 40A). Interestingly, all 3 partial responders had BRAF-mutant melanoma. One partial responder, who was receiving BVD-523 at a dose of 450 mg BID, had an approximate 70% reduction in the sum of target lesions from baseline, while the other partial responders showed reductions of 47.0% and 33.6%. Stable disease was demonstrated in 18 patients, with 6 having stable disease for more than 6 months, and 6 additional patients having stable disease for more than 3 months. In this study, 4 patients displayed progressive disease at first evaluation.

FIG. 40B shows computed tomography (CT) scans of 1 of the 3 partial responders (RECIST v1.1) who had progressed on prior vemurafenib and subsequent dabrafenib/trametinib treatment; a durable partial response was observed following dosing of BVD-523 600 mg BID for >300 days. BVD-523 was associated with a metabolic response using fluorodeoxyglucose-positive emission tomography (18F-FDG-PET) in 5 of 16 evaluable patients.

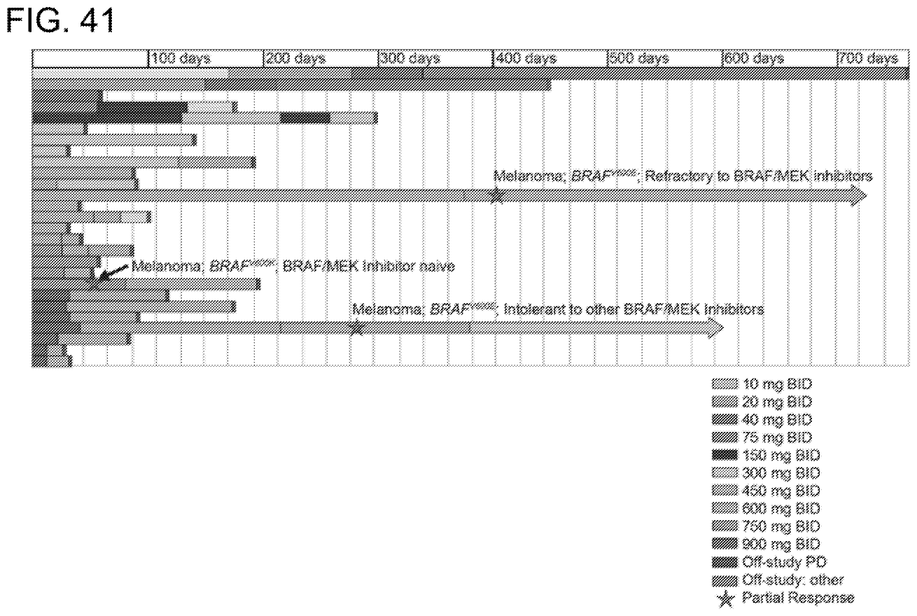

FIG. 41 depicts the time to response and the duration of response in the study population. The two patients who demonstrated responses to BVD-523 remained on study and continued with BVD-523 treatment as of the study cutoff date (>500 days); additionally, one patient with bronchoalveolar NSCLC (not enough tissue for molecular profiling) had been on treatment for >700 days with stable disease. Twenty-four of 27 patients (90%) discontinued treatment due to progressive disease ( 22/27, 82%) or other reasons ( 2/27, 7%). The mean duration of BVD-523 treatment before discontinuation was 4.7 months.

### Discussion

The present invention presents results from a first-in-human study evaluating the safety, pharmacokinetics, pharmacodynamics, and preliminary efficacy of BVD-523 in 27 patients with advanced solid tumors. In this dose-escalation study, oral treatment with BVD-523 resulted in both radiographic responses by RECIST v1.1 (3 partial responses) and prolonged disease stabilization in some patients, the majority of whom had been treated with ≥2 prior systemic therapies. Evidence of BVD-523-dependent inhibition of metabolic response in tumors was established in a subset of patients by imaging tumor uptake of 18F-glucose. Drug exposures increased linearly with increasing doses up to 600 mg BID, with exposures at 600 mg BID providing near complete 24/7 inhibition of ERK-dependent substrate (RSK-1) phosphorylation in an ex vivo whole blood assay. Furthermore, tolerability to BVD-523 was manageable when administered up to its MTD and RP2D, determined to be 600 mg BID.

BVD-523 was generally well tolerated, with manageable and reversible toxicity. The most common AEs were rash (usually acneiform), fatigue, and gastrointestinal side effects, including nausea, vomiting, and diarrhea. The safety profile of BVD-523 is consistent with its selective inhibition of the MAPK pathway; the AE profile shows considerable overlap with MEK inhibitor experience. However, toxicities associated with any targeted therapy may include dependence on both the specific mechanism and the degree of target inhibition as well as any off-target effects (Zelboraf [package insert] and Hauschild et al. 2012). Ongoing and future investigations will extend both the efficacy and safety profile demonstrated in this dose-escalation study, and will guide how the unique profile of the ERK inhibitor BVD-523 might be used as a single agent or in combination with other agents.

Durable responses by RAF and MEK inhibitors are often limited by intrinsic and eventual acquired resistance, with a common feature often involving reactivation of the ERK pathway (Poulikakos et al. 2011, Corcoran et al. 2010, Nazarian et al. 2010, Shi et al. 2014, Johannessen et al. 2010, Wagle et al. 2011, Wagle et al. 2014, Ahronian et al. 2015 and Paraiso et al. 2010). Thus, ERK inhibition with BVD-523 alone or in combination with other MAPK signaling pathway inhibitors may have the potential to delay the development of resistance to existing therapies and to benefit a broader patient population. That ERK inhibitors, including BVD-523, retain their potency in BRAF- and MEK-resistant cell lines provide preclinical evidence for the use of ERK inhibitors in patients with acquired resistance to standard of care (BRAF/MEK combination therapy) See, e.g., Examples 9-16. Importantly, in this study, a patient whose cancer had progressed after experiencing stable disease when treated initially with a BRAF inhibitor (vemurafenib) and subsequently with a combination of BRAF and MEK inhibitors (dabrafenib/trametinib) had a partial response when receiving single-agent BVD-523. This patient has remained on-study for a total of 708 days, as of the cutoff date of the study reported herein. Based in part on the antitumor effects observed in this patient, the FDA has designated as a Fast Track development program the investigation of BVD-523 for the treatment of patients with unresectable or metastatic BRAF^{V600} mutation-positive melanoma that is refractory to or has progressed following treatment with a BRAF and/or MEK inhibitor(s). Precise definition of exactly how BVD-523 might best support patient care (eg, as a single agent or in various combinations) requires additional clinical studies.

In summary, the present examples present data from an initial data from the dose escalation portion of a phase I study evaluating BVD-523, a novel first-in-class ERK inhibitor, as a treatment for patients with advanced cancers. Continuous, twice-daily oral treatment with BVD-523 resulted in antitumor effects in several patients, including patients either naïve to or having progressed on available MAPK pathway-targeted therapies. BVD-523 was generally well tolerated in this advanced cancer patient population and toxicities were manageable; the MTD and RP2D were 600 mg BID. BVD-523 exposures increased linearly up to the RP2D and robust pharmacodynamics effects were evident at this dose level. An expansion of this phase I clinical study is currently underway to confirm and extend the observations made in the dose-escalation phase. Specifically, patients are being enrolled into molecularly classified expansion cohorts (e.g., NRAS, BRAF, MEK or ERK alterations) across various tumor histologies. Furthermore, expansion cohorts are evaluating the use of BVD-523 in patients with cancer who are either naïve to available MAPK pathway therapies or those whose disease has progressed on such treatments.

### DOCUMENTS

ABSALAN, Farnaz, Mostafa Ronaghi (2008). Molecular Inversion Probe Assay. Methods in Molecular Biology 396. Humana Press. pp. 315-330.
AHRONIAN L G, Sennott E M, Van Allen E M, Wagle N, Kwak E L, Faris J E, et al. Clinical acquired resistance to RAF inhibitor combinations in BRAF-mutant colorectal cancer through MAPK pathway alterations. Cancer Discov 2015; 5:358-67.
ARCILA M E, Drilon A, Sylvester B E, Lovly C M, Borsu L, Reva B, et al. MAP2K1 (MEK1) mutations define a distinct subset of lung adenocarcinoma associated with smoking. Clin Cancer Res 2015; 21:1935-43.
ARONOV A M, Baker C, Bemis G W, Cao J, Chen G, Ford P J, et al. Flipped out: structure-guided design of selective pyrazolylpyrrole ERK inhibitors. J Med Chem 2007; 50:1280-7.
ARONOV A M, Tang Q, Martinez-Botella G, Bemis G W, Cao J, Chen G, et al. Structure-guided design of potent and selective pyrimidylpyrrole inhibitors of extracellular signal-regulated kinase (ERK) using conformational control. J Med Chem 2009; 52:6362-8.
ARRINGTON A K, Heinrich E L, Lee W, Duldulao M, Patel S, Sanchez J, et al. Prognostic and predictive roles of KRAS mutation in colorectal cancer. Int J Mol Sci 2012; 13:12153-68.
CARGNELLO M, Roux P P. Activation and function of the MAPKs and their substrates, the MAPK-activated protein kinases. Microbiol Mol Biol Rev 2011; 75:50-83.
CARLINO M S, Fung C, Shahheydari H, Todd J R, Boyd S C, Irvine M, et al. Preexisting MEK1P124 mutations diminish response to BRAF inhibitors in metastatic melanoma patients. Clin Cancer Res 2015; 21:98-105.
CHAPMAN P B, Hauschild A, Robert C, Haanen J B, Ascierto P, Larkin J, et al. Improved survival with vemurafenib in melanoma with BRAF V600E mutation. N Engl J Med 2011; 364:2507-16.
CORCORAN, R. B., et al. BRAF gene amplification can promote acquired resistance to MEK inhibitors in cancer cells harboring the BRAF V600E mutation. Sci Signal (2010); 3 (149): ra84.
DAI, B., et al. STAT3 mediates resistance to MEK inhibitor through microRNA miR-17. Cancer Res (2011); 71:3658-3668.
DAVIES H, Bignell G R, Cox C, Stephens P, Edkins S, Clegg S, et al. Mutations of the BRAF gene in human cancer. Nature 2002; 417:949-54.
DESCHENES-SIMARD X, Kottakis F, Meloche S, Ferbeyre G. ERKs in cancer: friends or foes? Cancer Res 2014; 74:412-9.
DOBRZYCKA B, Terlikowski S J, Kowalczuk O, Niklinska W, Chyczewski L, Kulikowski M. Mutations in the KRAS gene in ovarian tumors. Folia Histochem Cytobiol 2009; 47:221-4.
EMERY, C. M., et al. MEK1 mutations confer resistance to MEK and B-RAF inhibition. PNAS (2009); 106 (48): 20411-6.
FEDOROV O, Niesen F H, Knapp S. Kinase inhibitor selectivity profiling using differential scanning fluorimetry. Methods Mol Biol 2012; 795:109-18.
FERNÁNDEZ-MEDARDE A, Santos E. Ras in cancer and developmental diseases. Genes Cancer 2011; 2:344-58.
FLAHERTY K T, Infante J R, Daud A, Gonzalez R, Kefford R F, Sosman J, et al. Combined BRAF and MEK inhibition in melanoma with BRAF V600 mutations. N Engl J Med 2012; 367:1694-703.
GOETZ E M, Ghandi M, Treacy D J, Wagle N, Garraway L A. ERK mutations confer resistance to mitogen-activated protein kinase pathway inhibitors. Cancer Res 2014; 74:7079-89.
GOLLOB J A, Wilhelm S, Carter C, Kelley S L. Role of Raf kinase in cancer: therapeutic potential of targeting the Raf/MEK/ERK signal transduction pathway. Semin Oncol 2006; 33:392-406.
GREGER, James G., et al. “Combinations of BRAF, MEK, and PI3K/mTOR inhibitors overcome acquired resistance to the BRAF inhibitor GSK2118436 dabrafenib, mediated by NRAS or MEK mutations.” Molecular cancer therapeutics 11.4 (2012): 909-920.
GROENENDIJK F H, Bernards R. Drug resistance to targeted therapies: deja vu all over again. Mol Oncol 2014; 8:1067-83.
HALL R D, Kudchadkar R R. BRAF mutations: signaling, epidemiology, and clinical experience in multiple malignancies. Cancer Control 2014; 21:221-30.
HARDENBOL, P., et al. Multiplexed genotyping with sequence-tagged molecular inversion probes. Nat. Biotechnol. 2003, no. 21, p. 673-678.
HATZIVASSILIOU, Georgia, et al. “RAF inhibitors prime wild-type RAF to activate the MAPK pathway and enhance growth.” Nature 464.7287 (2010): 431-435.
HATZIVASSILIOU G, Liu B, O'Brien C, Spoerke J M, Hoeflich K P, Haverty P M, et al. ERK inhibition overcomes acquired resistance to MEK inhibitors. Mol Cancer Ther 2012; 11:1143-54.
HAUSCHILD A, Grob J-J, Demidov L V, Jouary T, Gutzmer R, Millward M, et al. Dabrafenib in BRAF-mutated metastatic melanoma: a multicentre, open-label, phase 3 randomised controlled trial. Lancet 2012; 380:358-65.
HAYES T K, Neel N F, Hu C, Gautam P, Chenard M, Long B, et al. Long-Term ERK Inhibition in KRAS-Mutant Pancreatic Cancer Is Associated with MYC Degradation and Senescence-like Growth Suppression. Cancer Cell 2016; 29:75-89.
HEZEL A F, Noel M S, Allen J N, Abrams T A, Yurgelun M, Faris J E, et al. Phase II study of gemcitabine, oxaliplatin in combination with panitumumab in KRAS wild-type unresectable or metastatic biliary tract and gallbladder cancer. Br J Cancer 2014; 111:430-6.
JHA S, Morris E J, Hruza A, Mansueto M S, Schroeder G, Arbanas J, et al. Dissecting therapeutic resistance to ERK inhibition. Mol Cancer Ther 2016; 15:548-59.
JOHANNESSEN, C. M., et al. COT/MAP3K8 drives resistance to RAF inhibition through MAP kinase pathway reactivation. Nature (2010); 468 (7326): 968-972.
JOHNSON D B, Menzies A M, Zimmer L, Eroglu Z, Ye F, Zhao S, et al. Acquired BRAF inhibitor resistance: A multicenter meta-analysis of the spectrum and frequencies, clinical behaviour, and phenotypic associations of resistance mechanisms. Eur J Cancer 2015; 51:2792-9.
KANDA M, Matthaei H, Wu J, Hong S M, Yu J, Borges M, et al. Presence of somatic Mutations in most early-stage pancreatic intraepithelial neoplasia. Gastroenterology 2012; 142:730-733.
KHATTAK M, Fisher R, Turajlic S, Larkin J. Targeted therapy and immunotherapy in advanced melanoma: an evolving paradigm. Ther Adv Med Oncol 2013; 5:105-18.
KING, Alastair J., et al. “Dabrafenib; preclinical characterization, increased efficacy when combined with trametinib, while BRAF/MEK tool combination reduced skin lesions.” PloS one 8.7 (2013): e67583.
LARKIN J, Ascierto P A, Dreno B, Atkinson V, Liszkay G, Maio M, et al. Combined vemurafenib and cobimetinib in BRAF-mutated melanoma. N Engl J Med 2014; 371:1867-76.
LITTLE, A. S., et al., Amplification of the Driving Oncogene, KRAS or BRAF, Underpins Acquired Resistance to MEK1/2 Inhibitors in Colorectal Cancer Cells. Sci. Signal. 4, ra17 (2011).
LIU, Dingxie, et al. “BRAF V600E maintains proliferation, transformation, and tumorigenicity of BRAF-mutant papillary thyroid cancer cells.” Journal of Clinical Endocrinology & Metabolism 92.6 (2007): 2264-2271.
LIU B, Fu L, Zhang C, Zhang L, Zhang Y, Ouyang L, et al. Computational design, chemical synthesis, and biological evaluation of a novel ERK inhibitor (BL-EI001) with apoptosis-inducing mechanisms in breast cancer. Oncotarget 2015; 6:6762-75.
LONG G V, Fung C, Menzies A M, Pupo G M, Carlino M S, Hyman J, et al. Increased MAPK reactivation in early resistance to dabrafenib/trametinib combination therapy of BRAF-mutant metastatic melanoma. Nat Commun 2014; 5:5694.
LONG G V, Stroyakovskiy D, Gogas H, Levchenko E, de Braud F, Larkin J, et al. Dabrafenib and trametinib versus dabrafenib and placebo for Val600 BRAF-mutant melanoma: a multicentre, double-blind, phase 3 randomised controlled trial. Lancet 2015; 386:444-51.
MANANDHAR S P, Hildebrandt E R, Schmidt W K. Small-molecule inhibitors of the Rce1p CaaX protease. J Biomol Screen. 2007; 12 (7): 983-993.
MASSEY P R, Prasad V, Figg W D, Fojo T. Multiplying therapies and reducing toxicity in metastatic melanoma. Cancer Biol Ther 2015; 16:1014-8.
MAURER, T, Garrenton, L S, Oh, A, Pitts, K, Anderson, D J, Skelton, N J, Fauber, B P, Pan, B, Malek, S, Stokoe, D, Ludlam, MJC, Bowman, K K, Wu, J, Giannetti, A M, Starovasnik, M A, Mellman, I, Jackson, P K, Rudolph, J, Wang, W, Fang, G. Small-molecule ligands bind to a distinct pocket in Ras and inhibit SOS-mediated nucleotide exchange activity. PNAS. 2012; 109 (14): 5299-304.
MCARTHUR G A, Chapman P B, Robert C, Larkin J, Haanen J B, Dummer R, et al. Safety and efficacy of vemurafenib in BRAFv600E and BRAFv600K mutation-positive melanoma (BRIM-3): extended follow-up of a phase 3, randomised, open-label study. Lancet Oncol 2014; 15:323-32.
MEKINIST [package insert]. Research Triangle Park, N C: GlaxoSmithKline; 2014.
METZKER, Emerging technologies in DNA sequencing Genome Res. 2005. 15:1767-1776.
MITTAL, Rohit et al. “The acetyltransferase activity of the bacterial toxin YopJ of Yersinia is activated by eukaryotic host cell inositol hexakisphosphate.” Journal of Biological Chemistry 285.26 (2010): 19927-19934.
MORRIS E J, Jha S, Restaino C R, Dayananth P, Zhu H, Cooper A, et al. Discovery of a novel ERK inhibitor with activity in models of acquired resistance to BRAF and MEK inhibitors. Cancer Discov 2013; 3:742-50.
NAZARIAN, R., et al. Melanomas acquire resistance to B-RAF (V600E) inhibition by RTK or N-RAS upregulation. Nature. 2010; 468 (7326): 973-977.
NIKOLAEV S I, Rimoldi D, Iseli C, Valsesia A, Robyr D, Gehrig C, et al. Exome sequencing identifies recurrent somatic MAP2K1 and MAP2K2 mutations in melanoma. Nat Genet 2012; 44:133-9.
NILSSON, M., et al. Padlock probes: circularizing oligonucleotides for localized DNA detection. Science. 1994, no. 265, p. 2085-2088.
O'HARA A J, Bell D W. The genomics and genetics of endometrial cancer. Adv Genomics Genet 2012; 2012:33-47.
OJESINA A I, Lichtenstein L, Freeman S S, Pedamallu C S, Imaz-Rosshandler I, Pugh T J, et al. Landscape of genomic alterations in cervical carcinomas. Nature 2014; 506:371-5.
OTA et al., Single nucleotide polymorphism detection by polymerase chain reaction-restriction fragment length polymorphism. Nat Protoc. 2007; 2 (11): 2857-64.
PARAISO KHT, Fedorenko I V, Cantini L P, Munko A C, Hall M, Sondak V K, et al. Recovery of phospho-ERK activity allows melanoma cells to escape from BRAF inhibitor therapy. Br J Cancer 2010; 102:1724-30.
PATGIRI A, Yadav, K K, Arora, P S, Bar-Sagi, D. An orthosteric inhibitor of the Ras-Sos interaction. Nat Chem Biol. 2011; 7:585-587.
PENNYCUICK A, Simpson T, Crawley D, Lal R, Santis G, Cane P, et al. Routine EGFR and KRAS mutation analysis using COLD-PCR in non-small cell lung cancer. Int J Clin Pract 2012; 66:748-52.
PORTER S B, Hildebrandt E R, Breevoort S R, Mokry D Z, Dore T M, Schmidt W K. Inhibition of the CaaX proteases Rce1p and Ste24p by peptidyl (acyloxy)methyl ketones. Biochim Biophys Acta. 2007; 1773 (6): 853-862.
POULIKAKOS P I, Persaud Y, Janakiraman M, Kong X, Ng C, Moriceau G, et al. RAF inhibitor resistance is mediated by dimerization of aberrantly spliced BRAF (V600E). Nature 2011; 480:387-90.
QUEIROLO P, Picasso V, Spagnolo F. Combined BRAF and MEK inhibition for the treatment of BRAF-mutated metastatic melanoma. Cancer Treat Rev 2015; 41:519-26.
RASOLA A, Sciacovelli M, Chiara F, Pantic B, Brusilow W S, Bernardi P. Activation of mitochondrial ERK protects cancer cells from death through inhibition of the permeability transition. Proc Natl Acad Sci USA 2010; 107:726-31.
RIZOS H, Menzies A M, Pupo G M, Carlino M S, Fung C, Hyman J, et al. BRAF inhibitor resistance mechanisms in metastatic melanoma: spectrum and clinical impact. Clin Cancer Res 2014; 20:1965-77.
ROBERT C, Karaszewska B, Schachter J, Rutkowski P, Mackiewicz A, Stroiakovski D, et al. Improved overall survival in melanoma with combined dabrafenib and trametinib. N Engl J Med 2015; 372:30-9.
ROMEO Y, Zhang X, Roux P P. Regulation and function of the RSK family of protein kinases. Biochem J 2012; 441:553-69.
RUDOLPH J, Xiao Y, Pardi A, Ahn N G. Slow inhibition and conformation selective properties of extracellular signal-regulated kinase 1 and 2 inhibitors. Biochemistry 2015; 54:22-31.
SHAUL Y D, Seger R. The MEK/ERK cascade: from signaling specificity to diverse functions. Biochim Biophys Acta 2007; 1773:1213-26.
SHI H, Hugo W, Kong X, Hong A, Koya R C, Moriceau G, et al. Acquired resistance and clonal evolution in melanoma during BRAF inhibitor therapy. Cancer Discov 2014; 4:80-93.
SHIMA, F, Yoshikawa, Y, Ye, M, Araki, M, Matsumoto, S, Liao, J, Hu, L, Sugimoto, T, Ijiri, Y, Takeda, A, Nishiyama, Y, Sato, C, Muraoka, S, Tamura, A, Osoda, T, Tsuda, K-I, Miyakawa, T, Fukunishi, H, Shimada, J, Kumasaka, Yamamoto, M, Kataoka, T. In silico discovery of small-molecule Ras inhibitors that display antitumor activity by blocking the Ras-effector interaction. PNAS. 2013; 110 (20): 8182-7.
SCHUBBERT S, Shannon K, Bollag G. Hyperactive Ras in developmental disorders and cancer. Nat Rev Cancer 2007; 7:295-308.
SUN C, Hobor S, Bertotti A, Zecchin D, Huang S, Galimi F, et al. Intrinsic resistance to MEK inhibition in KRAS mutant lung and colon cancer through transcriptional induction of ERBB3. Cell Rep 2014; 7:86-93.
TAFLINAR [package insert]. Research Triangle Park, N C: GlaxoSmithKline; 2015.
TRUNZER K, Pavlick A C, Schuchter L, Gonzalez R, McArthur G A, Hutson T E, et al. Pharmacodynamic effects and mechanisms of resistance to vemurafenib in patients with metastatic melanoma. J Clin Oncol 2013; 31:1767-74.
VILLANUEVA, J., et al. Acquired resistance to BRAF inhibitors mediated by a RAF kinase switch in melanoma can be overcome by cotargeting MEK and IGF-1R/PI3K. Cancer Cell. 2010; 18:683-695.
WAGLE, N., et al. Dissecting therapeutic resistance to RAF inhibition in melanoma by tumor genomic profiling. Journal of Clinical Oncology 2011; 29 (22): 3085-3096.
WAGLE N, Van Allen E M, Treacy D J, Frederick D T, Cooper Z A, Taylor-Weiner A, et al. MAP kinase pathway alterations in BRAF-mutant melanoma patients with acquired resistance to combined RAF/MEK inhibition. Cancer Discov 2014; 4:61-8.
Wainstein E, Seger R. The dynamic subcellular localization of ERK: mechanisms of translocation and role in various organelles. Curr Opin Cell Biol 2016; 39:15-20.
WANG, H., et al. Identification of the MEK1 (F129L) activating mutation as a potential mechanism of acquired resistance to MEK inhibition in human cancers carrying the B-RAF V600E mutation. Cancer Res (2011); 71 (16): 5535-45.
YANG W, Soares J, Greninger P, Edelman E J, Lightfoot H, Forbes S, et al. Genomics of Drug Sensitivity in Cancer (GDSC): a resource for therapeutic biomarker discovery in cancer cells. Nucleic Acids Res 2013; 41: D955-D961.
YAO Z, Torres N M, Tao A, Gao Y, Luo L, Li Q, et al. BRAF Mutants Evade ERK-Dependent Feedback by Different Mechanisms that Determine Their Sensitivity to Pharmacologic Inhibition. Cancer Cell 2015; 28:370-83.
YOHE S. Molecular genetic markers in acute myeloid leukemia. J Clin Med 2015; 4:460-78.
ZELBORAF [package insert]. South San Francisco, CA: Genentech USA, Inc.; 2015.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A composition for treating or ameliorating effects of a cancer in a subject, comprising an effective amount of (i) BVD-523 (ulixertinib) that has a structure of 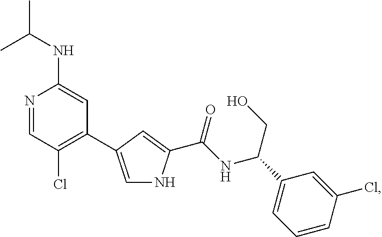 [SMILES: CC(C)Nc1cc(-c2c[nH]c(C(=O)N[C@H](CO)c3cccc(Cl)c3)c2)c(Cl)cn1]
or a pharmaceutically acceptable salt thereof, and (ii) SCH772984, and optionally a pharmaceutically acceptable carrier, adjuvant, or vehicle.

2. The composition of claim 1, wherein the subject has at least one mutation on genes selected from the group consisting of KRAS, NRAS, HRAS, BRAF, MEK1, and combinations thereof.

3. The composition of claim 2, wherein the subject has a single mutation on BRAF, which is BRAF^{V600E}.

4. The composition of claim 2, wherein the subject has a double mutation on BRAF and MEK1, which is BRAF^{V600E}::MEK1^{Q56P}.

5. The composition of claim 2, wherein the subject has a single mutation on KRAS occurs at KRAS^{G12} or KRAS^{G13}.

6. The composition of claim 1, wherein the cancer is selected from the group consisting of pancreatic cancer, papillary thyroid cancer, colorectal cancer, non-small cell lung cancer, ovarian cancer, endometrioid endometrial cancer, and melanoma.

7. The composition of claim 1, wherein the cancer is melanoma.

8. The composition of claim 1, wherein the cancer is pancreatic cancer.

9. The composition of claim 1, wherein the subject is selected from the group consisting of humans, primates, farm animals, and domestic animals.

10. The composition of claim 1, wherein the subject is a human.

11. The composition of claim 1, wherein the subject has received prior therapy selected from a RAS inhibitor, a RAF inhibitor, a MEK inhibitor, and combinations thereof.

12. The composition of claim 1, comprising at least 100 mg of BVD-523 or a pharmaceutically acceptable salt thereof.

13. The composition of claim 1, comprising 600 mg of BVD-523 or a pharmaceutically acceptable salt thereof.

14. The composition of claim 1, further comprising an effective amount of at least one additional anti-cancer agent.
